(19) United States
(12) Patent Application Publication
(10) Pub. No.: US 2026/0200849 A1
(43) Pub. Date: Jul. 16, 2026

(54) CATIONIC LIPIDS AND PREPARATION METHOD THEREOF

(71) Applicant: RiboX Therapeutics HK Limited, Hong Kong (CN)

(72) Inventors: Roi Mashiach, Hong Kong (CN); Niv Cohen, Hong Kong (CN); Moshe Goldmeier, Hong Kong (CN); Aviad Sela, Hong Kong (CN); Fei Lin, Hong Kong (CN)

(73) Assignee: RiboX Therapeutics HK Limited, Hong Kong (CN)

(21) Appl. No.: 19/444,016

(22) Filed: Jan. 8, 2026

Related U.S. Application Data
(63) Continuation of application No. PCT/CN2025/097110, filed on May 26, 2025.

(30) Foreign Application Priority Data
May 24, 2024 (WO) PCT/CN2024/095182
Oct. 28, 2024 (WO) PCT/CN2024/127721
Nov. 11, 2024 (WO) PCT/CN2024/131326
Nov. 13, 2024 (WO) PCT/CN2024/131749
May 21, 2025 (WO) PCT/CN2025/096251

(51) Int. Cl. C07D 211/26; C07D 223/04

(52) U.S. Cl. CPC C07D 211/26; C07D 223/04

(57) ABSTRACT

The present invention provides cationic lipids and lipid nanoparticle formulations comprising these lipids, alone or in combination with other lipids. These lipid nanoparticles may be formulated with nucleic acids to facilitate their intracellular delivery both in vitro and for therapeutic applications. The present invention also provides methods of chemical synthesis of these lipids.

### TECHNICAL FIELD

The present invention provides novel cationic lipids and compositions such as lipid nanoparticles comprising such cationic lipids, which can be used for intracellular delivery of therapeutic agents. These lipid nanoparticles may be formulated with nucleic acids to facilitate their intracellular delivery both in vitro and for in vivo therapeutic applications. The present invention also provides methods of chemical synthesis of the cationic lipids.

### REFERENCE TO AN ELECTRONIC SEQUENCE LISTING

The contents of the electronic sequence listing (P2025TC3328_Sequence Listing.xml; Size: 8,192 bytes; and Date of Creation: May 25, 2025) is herein incorporated by reference in its entirety.

### BACKGROUND

Research and development of therapeutic nucleic acids including circular RNA (cRNA), small interfering RNA (siRNA), microRNA (miRNA), antisense oligo nucleotides, messenger RNA (mRNA) as pharmaceutical drug have spurred exponential growth in the last decade. Drugs based on nucleic acids, which include large nucleic acid molecules, have to be delivered to the proper cellular compartment in order to be effective.

Cationic lipids have proved to be excellent carriers of nucleic acids to treat varies diseases in gene therapy applications. Lipid nanoparticles (LNPs) formed from cationic lipids and other co-lipids including but not limited to cholesterol, DSPC and PEGylated lipids encapsulated oligonucleotides which protect them from degradation and facilitate the cellular uptake.

Despites these efforts, there remains a need for improved lipid nanoparticle formulations that provide high potency following administration and that allow for the administration of various types of nucleic acids.

### SUMMARY OF THE INVENTION

In some aspects, the present invention provides a cationic lipid represented by the structure of formula (I)-1:

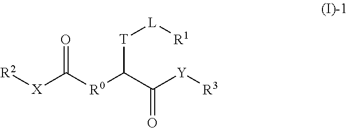

or a salt, hydrate, solvate, polymorph, optical isomer, geometrical isomer, enantiomer, diastereomer, tautomer, isotope labeled compound or mixtures thereof,
wherein:
L, X and Y are each independently selected from the group consisting of a direct bond, —O—, —C(═O)—, —OC(═O)—, —C(═O)O—, —OC(═O)O—, —C(═O)S—, —O—C(═O)S—, —NH—, —NHC(═O)—, —O—C(═O)—NH—, —NH—C(═O)—O—, —NH—C(═O)—NH—, —NH—C(═O)—S—, —NH(S═O)—, —NHS(═O)₂—, —S—, —S(═O)—, —S(═O)₂—, —W—W′—, —C₁₋₆ alkylene-, —C₂₋₆ alkenylene-, —W—C₁₋₆ alkylene-, —W—C₂₋₆ alkenylene-, —C₁₋₆ alkylene-W—, —C₂₋₆ alkenylene-W—, —W—W′—C₁₋₆ alkylene-, —C₁₋₆ alkylene-W—W′—, —W—W′—C₁₋₆ alkenylene-, —C₂₋₆ alkenylene-W—W′—, —W—C₁₋₆ alkylene-W′— and —W—C₂₋₆ alkenylene-W′—, wherein the alkylene and alkenylene are optionally further interrupted by one or more W;
W and W′, at each occurrence, are each independently selected from the group consisting of —O—, —C(═O)—, —OC(═O)—, —C(═O)O—, —OC(═O)O—, —C(═O)S—, —O—C(═O)S—, —NH—, —NHC(═O)—, —O—C(═O)—NH—, —NH—C(═O)—O—, —NH—C(═O)—NH—, —NH—C(═O)—S—, —NH(S=O)—, —NHS(═O)₂—, —S—, —S(═O)—, —S(═O)₂—, —C₁₋₆ alkylene-, and —C₂₋₆ alkenylene-;
T is —(CH₂)ₘ— or

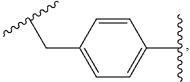 [SMILES: *Cc1ccc(*)cc1]

and m is 1, 2, 3 or 4;
R0 is selected from O and NH, provided that when R0 is O, then L must comprise a nitrogen atom; when R0 is NH, then L must comprise an oxygen atom;
R¹, R² and R³ are each independently selected from the group consisting of R^{H}, R^{T1} and R^{T2}, provided that one of R¹, R² and R³ is R^{H};
R^{H} is selected from the group consisting of:
   (a) NR⁴R⁵ wherein R⁴ and R⁵ are each independently H, C₁-C₁₀ alkyl (e.g., C₁-C₆ alkyl), —NH₂, halogen, —OH, a 3- to 10-membered cyclic ring or C₆₋₁₂ aralkyl, wherein the C₁-C₁₀ alkyl (e.g., C₁-C₆ alkyl), 3- to 10-membered cyclic ring or C₆₋₁₂ aralkyl is optionally substituted with one or more substituents selected from the group consisting of —NH₂, halogen, —OH, —C₁₋₆ alkyl and a 3- to 4-membered cyclic ring; or R⁴ and R⁵ together with the nitrogen to which they are attached form a 4- to 10-membered heterocyclic ring or 5- to 10-membered heteroaromatic ring, optionally containing one or more additional heteroatoms selected from the group consisting of O, N and S;
   (b) the side chain of a natural or unnatural amino acid;
   (c) a 3- to 10-membered cyclic ring, for example a C₆₋₁₂ aromatic ring, a 3- to 10-membered heterocyclic ring, a 5- to 10-membered heteroaromatic ring containing one or more heteroatoms selected from the group consisting of O, N and S, or a fused ring (e.g., a 4- to 10-membered fused ring), wherein the above cyclic ring is optionally substituted with one or more substituents selected from the group consisting of —NH₂, halogen, —OH, —C₁₋₆ alkyl, —C₁₋₆ alkylene-OH, and a 3- to 4-membered cyclic ring; and
   (d) —OH, or —C₁₋₁₀ alkyl optionally substituted with one or more substituents selected from the group consisting of —NH₂, halogen, —OH, —C₁₋₆ alkyl and a 3- to 4-membered cyclic ring;
preferably, R^{H} is selected from the group consisting of:
   (a) NR⁴R⁵ wherein R⁴ and R⁵ are each independently H, C₁-C₆ alkyl or C₆₋₁₂ aralkyl optionally substituted with —NH₂; or R⁴ and R⁵ together with the nitrogen to which they are attached form a 4- to 10-membered heterocyclic ring or 5- to 10-membered heteroaromatic ring, optionally containing one or more additional heteroatoms selected from the group consisting of O, N and S;
   (b) the side chain of a natural or unnatural amino acid; and
   (c) a 3- to 10-membered heterocyclic ring or 5- to 10-membered heteroaromatic ring containing one or more heteroatoms selected from the group consisting of O, N and S;
   (d) —OH or —C₁₋₁₀ alkyl optionally substituted with one or more substituents selected from the group consisting of —NH₂, halogen, —OH, —C₁₋₆ alkyl and a 3- to 4-membered cyclic ring;
R^{T1} and R^{T2} are each independently selected from the group consisting of:
   (a) C₁₀-C₂₂ alkyl;
   (b) C₁₀-C₂₂ alkenyl;
   (c) C₁₀-C₂₂ alkynyl;
   (d) C₄-C₁₅ alkylene-Z—C₄-C₂₂ alkyl; and
   (e) C₄-C₁₅ alkylene-Z—C₄-C₂₂ alkenyl;
   Z is —O—C(═O)—, —C(═O)—O— or —O—.

In some aspects, the present invention provides a cationic lipid represented by the structure of formula (I)-1:

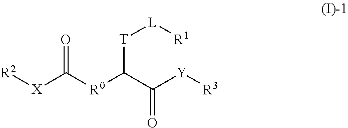

or a salt, hydrate, solvate, polymorph, optical isomer, geometrical isomer, enantiomer, diastereomer, tautomer, isotope labeled compound or mixtures thereof,
wherein:
L, X and Y are each independently selected from the group consisting of a direct bond, —O—, —C(═O)—, —OC(═O)—, —C(═O)O—, —OC(═O)O—, —C(═O)S—, —O—C(═O)S—, —NH—, —NHC(═O)—, —O—C(═O)—NH—, —NH—C(═O)—O—, —NH—C(═O)—NH—, —NH—C(═O)—S—, —NH(S=O)—, —NHS(═O)₂—, —S—, —S(═O)—, —S(═O)₂—, —W—W′—, —C₁₋₆ alkylene-, —C₂₋₆ alkenylene-, —W—C₁₋₆ alkylene-, —W—C₂₋₆ alkenylene-, —C₁₋₆ alkylene-W—, —C₂₋₆ alkenylene-W—, —W—W′—C₁₋₆ alkylene-, —C₁₋₆ alkylene-W—W′—, —W—W′—C₁₋₆ alkenylene-, —C₂₋₆ alkenylene-W—W′—, —W—C₁₋₆ alkylene-W′— and —W—C₂₋₆ alkenylene-W′—, wherein the alkylene and alkenylene are optionally further interrupted by one or more W;
W and W′, at each occurrence, are each independently selected from the group consisting of —O—, —C(═O)—, —OC(═O)—, —C(═O)O—, —OC(═O)O—, —C(═O)S—, —O—C(═O)S—, —NH—, —NHC(═O)—, —O—C(═O)—NH—, —NH—C(═O)—O—, —NH—C(═O)—NH—, —NH—C(═O)—S—, —NH(S=O)—, —NHS(═O)₂—, —S—, —S(═O)—, —S(═O)₂—, —C₁₋₆ alkylene-, and —C₂₋₆ alkenylene-;
T is —(CH₂)ₘ—, and m is 1;
R⁰ is selected from 0 and NH, provided that when R⁰ is 0, then L must comprise a nitrogen atom; when R⁰ is NH, then L must comprise an oxygen atom;
R¹, R² and R³ are each independently selected from the group consisting of R^{H}, R^{T1} and R^{T2}, provided that one of R¹, R² and R³ is R^{H};
R^{H} is selected from the group consisting of:
   (a) NR⁴R⁵ wherein R⁴ and R⁵ are each independently H, C₁-C₁₀ alkyl (e.g., C₁-C₆ alkyl), —NH₂, halogen, —OH, a 3- to 10-membered cyclic ring or C₆₋₁₂ aralkyl, wherein the C₁-C₁₀ alkyl (e.g., C₁-C₆ alkyl), 3- to 10-membered cyclic ring or C₆₋₁₂ aralkyl is optionally substituted with one or more substituents selected from the group consisting of —NH₂, halogen, —OH, —C₁₋₆ alkyl and a 3- to 4-membered cyclic ring; or R⁴ and R⁵ together with the nitrogen to which they are attached form a 4- to 10-membered heterocyclic ring or 5- to 10-membered heteroaromatic ring, optionally containing one or more additional heteroatoms selected from the group consisting of O, N and S;
   (b) the side chain of a natural or unnatural amino acid;
   (c) a 3- to 10-membered cyclic ring, for example a C₆₋₁₂ aromatic ring, a 3- to 10-membered heterocyclic ring, a 5- to 10-membered heteroaromatic ring containing one or more heteroatoms selected from the group consisting of O, N and S, or a fused ring (e.g., a 4- to 10-membered fused ring), wherein the above cyclic ring is optionally substituted with one or more substituents selected from the group consisting of —NH₂, halogen, —OH, —C₁₋₆ alkyl, —C₁₋₆ alkylene-OH, and a 3- to 4-membered cyclic ring; and
   (d) —OH, or —C₁₋₁₀ alkyl optionally substituted with one or more substituents selected from the group consisting of —NH₂, halogen, —OH, —C₁₋₆ alkyl and a 3- to 4-membered cyclic ring;
preferably, R^{H} is selected from the group consisting of:
   (a) NR⁴R⁵ wherein R⁴ and R⁵ are each independently H, C₁-C₆ alkyl or C₆₋₁₂ aralkyl optionally substituted with —NH₂; or R⁴ and R⁵ together with the nitrogen to which they are attached form a 4- to 10-membered heterocyclic ring or 5- to 10-membered heteroaromatic ring, optionally containing one or more additional heteroatoms selected from the group consisting of O, N and S;
   (b) the side chain of a natural or unnatural amino acid; and
   (c) a 3- to 10-membered heterocyclic ring or 5- to 10-membered heteroaromatic ring containing one or more heteroatoms selected from the group consisting of O, N and S;
   (d) —OH or —C₁₋₁₀ alkyl optionally substituted with one or more substituents selected from the group consisting of —NH₂, halogen, —OH, —C₁₋₆ alkyl and a 3- to 4-membered cyclic ring;
R^{T1} and R^{T2} are each independently selected from the group consisting of:
   (a) C₁₀-C₂₂ alkyl;
   (b) C₁₀-C₂₂ alkenyl;
   (c) C₁₀-C₂₂ alkynyl;
   (d) C₄-C₁₅ alkylene-Z—C₄-C₂₂ alkyl; and
   (e) C₄-C₁₅ alkylene-Z—C₄-C₂₂ alkenyl;
Z is —O—C(═O)—, —C(═O)—O— or —O—.

In some aspects, the present invention provides a cationic lipid represented by the structure of any of the following formulae:

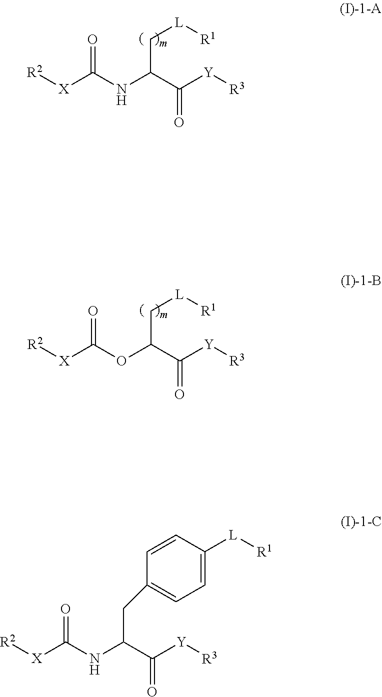

In some aspects, the present invention provides a cationic lipid represented by the structure of formula (I):

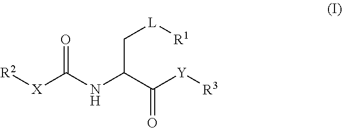

or a salt, hydrate, solvate, polymorph, optical isomer, geometrical isomer, enantiomer, diastereomer, tautomer, isotope labeled compound or mixtures thereof,
wherein:
L, X and Y are each independently selected from the group consisting of a direct bond, —O—, —C(═O)—, —OC(═O)—, —C(═O)O—, —NH—, —NHC(═O)—, —O—C(═O)—NH—, —NH—C(═O)—O—, —NH(S=O)—, —NHS(═O)₂—, —S—, —S(═O)—, —S(═O)₂—, —W—W′—, —C₁₋₆ alkylene-, —C₂₋₆ alkenylene-, —W—C₁₋₆ alkylene-, —W—C₂₋₆ alkenylene-, —C₁₋₆ alkylene-W—, —C₂₋₆ alkenylene-W—, —W—W′—C₁₋₆ alkylene-, —C₁₋₆ alkylene-W—W′—, —W—W′—C₁₋₆ alkenylene-, —C₂₋₆ alkenylene-W—W′—, —W—C₁₋₆ alkylene-W′— and —W—C₂₋₆ alkenylene-W′—, wherein the alkylene and alkenylene are optionally further interrupted by one or more W;
W and W′, at each occurrence, are each independently selected from the group consisting of —O—, —C(═O)—, —OC(═O)—, —C(═O)O—, —NH—, —NHC(═O)—, —O—C(═O)—NH—, —NH—C(═O)—O—, —NH(S=O)—, —NHS(═O)₂—, —S—, —S(═O)—, —S(═O)₂—, —C₁₋₆ alkylene-, and —C₂₋₆ alkenylene-;
R¹, R² and R³ are each independently selected from the group consisting of R^{H}, R^{T1} and R^{T2}, provided that one of R¹, R² and R³ is R^{H};
R^{H} is selected from the group consisting of:
   (a) NR⁴R⁵ wherein R and R are each independently H, C₁-C₁₀ alkyl (e.g., C₁-C₆ alkyl), —NH₂, halogen, —OH, a 3- to 10-membered cyclic ring or C₆₋₁₂ aralkyl, wherein the C₁-C₁₀ alkyl (e.g., C₁-C₆ alkyl), 3- to 10-membered cyclic ring or C₆₋₁₂ aralkyl is optionally substituted with one or more substituents selected from the group consisting of —NH₂, halogen, —OH, —C₁₋₆ alkyl and a 3- to 4-membered cyclic ring; or R⁴ and R⁵ together with the nitrogen to which they are attached form a 4- to 10-membered heterocyclic ring or 5- to 10-membered heteroaromatic ring, optionally containing one or more additional heteroatoms selected from the group consisting of O, N and S;
   (b) the side chain of a natural or unnatural amino acid;
   (c) a 3- to 10-membered cyclic ring, for example a C₆₋₁₂ aromatic ring, a 3- to 10-membered heterocyclic ring, a 5- to 10-membered heteroaromatic ring containing one or more heteroatoms selected from the group consisting of O, N and S, or a fused ring (e.g., a 4- to 10-membered fused ring), wherein the above cyclic ring is optionally substituted with one or more substituents selected from the group consisting of —NH₂, halogen, —OH, —C₁₋₆ alkyl, —C₁₋₆ alkylene-OH, and a 3- to 4-membered cyclic ring; and
   (d) —OH, or —C₁₋₁₀ alkyl optionally substituted with one or more substituents selected from the group consisting of —NH₂, halogen, —OH, —C₁₋₆ alkyl and a 3- to 4-membered cyclic ring;
preferably, R^{H} is selected from the group consisting of:
   (a) NR⁴R⁵ wherein R⁴ and R⁵ are each independently H, C₁-C₆ alkyl or C₆₋₁₂ aralkyl optionally substituted with —NH₂; or R⁴ and R⁵ together with the nitrogen to which they are attached form a 4- to 10-membered heterocyclic ring or 5- to 10-membered heteroaromatic ring, optionally containing one or more additional heteroatoms selected from the group consisting of O, N and S;
   (b) the side chain of a natural or unnatural amino acid; and
   (c) a 3- to 10-membered heterocyclic ring or 5- to 10-membered heteroaromatic ring containing one or more heteroatoms selected from the group consisting of O, N and S;
   (d) —OH or —C₁₋₁₀ alkyl optionally substituted with one or more substituents selected from the group consisting of —NH₂, halogen, —OH, —C₁₋₆ alkyl and a 3- to 4-membered cyclic ring;
R^{T1} and R^{T2} are each independently selected from the group consisting of:
   (a) C₁₀-C₂₂ alkyl;
   (b) C₁₀-C₂₂ alkenyl;
   (c) C₁₀-C₂₂ alkynyl;
   (d) C₄-C₁₅ alkylene-Z—C₄-C₂₂ alkyl; and
   (e) C₄-C₁₅ alkylene-Z—C₄-C₂₂ alkenyl;
   Z is —O—C(═O)—, —C(═O)—O— or —O—.

In some aspects, the present invention provides a cationic lipid, or a salt, hydrate, solvate, polymorph, optical isomer, geometrical isomer, enantiomer, diastereomer, tautomer, isotope labeled compound or mixtures thereof, wherein the cationic lipid is represented by the structure of formula (IA):

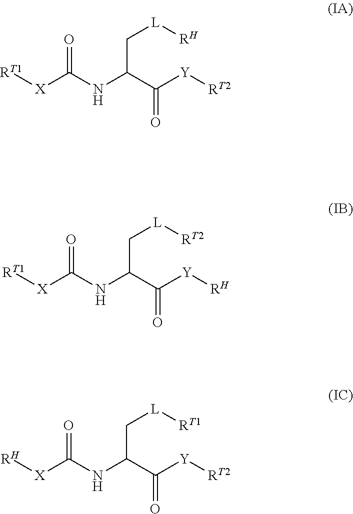

wherein each group is as defined herein.

In some aspects, the present invention provides a cationic lipid, or a salt, hydrate, solvate, polymorph, optical isomer, geometrical isomer, enantiomer, diastereomer, tautomer, isotope labeled compound or mixtures thereof, wherein the cationic lipid is represented by the structure of formula (II) or (III):

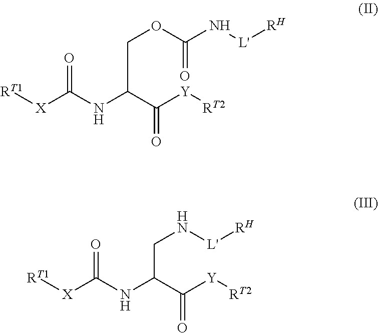

wherein:
L′ is —C₁₋₆ alkylene- or —C₂₋₆ alkenylene-;
R^{H} is selected from the group consisting of:
   (a) NR⁴R⁵ wherein R⁴ and R are each independently H, C₁-C₁₀ alkyl (e.g., C₁-C₆ alkyl), —NH₂, halogen, —OH, a 3- to 10-membered cyclic ring or C₆₋₁₂ aralkyl, wherein the C₁-C₁₀ alkyl (e.g., C₁-C₆ alkyl), 3- to 10-membered cyclic ring or C₆₋₁₂ aralkyl is optionally substituted with one or more substituents selected from the group consisting of —NH₂, halogen, —OH, —C₁₋₆ alkyl and a 3- to 4-membered cyclic ring; or R⁴ and R⁵ together with the nitrogen to which they are attached form a 4- to 10-membered heterocyclic ring or 5- to 10-membered heteroaromatic ring, optionally containing one or more additional heteroatoms selected from the group consisting of O, N and S;
   (b) the side chain of a natural or unnatural amino acid;
   (c) a 3- to 10-membered cyclic ring, for example a C₆₋₁₂ aromatic ring, a 3- to 10-membered heterocyclic ring, a 5- to 10-membered heteroaromatic ring containing one or more heteroatoms selected from the group consisting of O, N and S, or a fused ring (e.g., a 4- to 10-membered fused ring), wherein the above cyclic ring is optionally substituted with one or more substituents selected from the group consisting of —NH₂, halogen, —OH, —C₁₋₆ alkyl, —C₁₋₆ alkylene-OH, and a 3- to 4-membered cyclic ring; and
   (d) —OH, or —C₁₋₁₀ alkyl optionally substituted with one or more substituents selected from the group consisting of —NH₂, halogen, —OH, —C₁₋₆ alkyl and a 3- to 4-membered cyclic ring;
preferably, R^{H} is selected from the group consisting of:
   (a) NR⁴R⁵ wherein R⁴ and R⁵ are each independently H, C₁-C₆ alkyl or C₆₋₁₂ aralkyl optionally substituted with —NH₂; or R⁴ and R⁵ together with the nitrogen to which they are attached form a 4- to 10-membered heterocyclic ring or 5- to 10-membered heteroaromatic ring, optionally containing one or more additional heteroatoms selected from the group consisting of O, N and S;
   (b) the side chain of a natural or unnatural amino acid; and
   (c) a 3- to 10-membered heterocyclic ring or 5- to 10-membered heteroaromatic ring containing one or more heteroatoms selected from the group consisting of O, N and S; and
   (d) —OH, or —C₁₋₁₀ alkyl optionally substituted with one or more substituents selected from the group consisting of —NH₂, halogen, —OH, —C₁₋₆ alkyl and a 3- to 4-membered cyclic ring
R^{T1} and R^{T2} are each independently selected from the group consisting of:
   (a) C₁₀-C₂₂ alkyl;
   (b) C₁₀-C₂₂ alkenyl;
   (c) C₁₀-C₂₂ alkynyl;
   (d) C₄-C₁₅ alkylene-Z—C₄-C₂₂ alkyl; and
   (e) C₄-C₁₅ alkylene-Z—C₄-C₂₂ alkenyl;
Z is —O—C(═O)—, —C(═O)—O— or —O—.

In some aspects, the present invention provides a cationic lipid represented by the structure of formula (II):

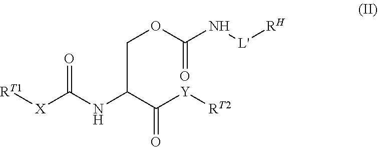

or a salt, hydrate, solvate, polymorph, optical isomer, geometrical isomer, enantiomer, diastereomer, tautomer, isotope labeled compound or mixtures thereof,
wherein
L′ is —(CH₂)ₙ—, and wherein n=1, 2 or 3, preferably, n=2;
R^{H} is —N(CH₃)₂, —N(C₂H₅)₂,

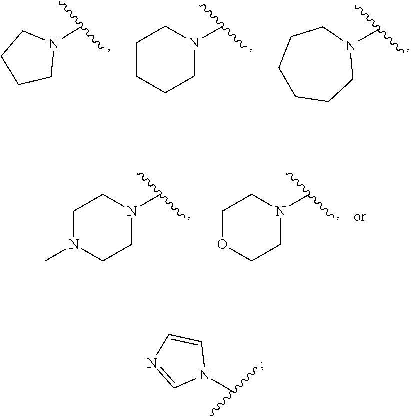

preferably, R^{H} is —N(CH₃)₂,

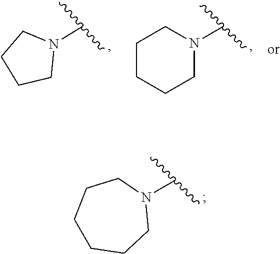 [SMILES: *N1CCCC1 ; *N1CCCCC1 ; *N1CCCCCC1]

X is —O—;
Y is —O—; and
R^{T1} and R^{T2} are each independently selected from the group consisting of:

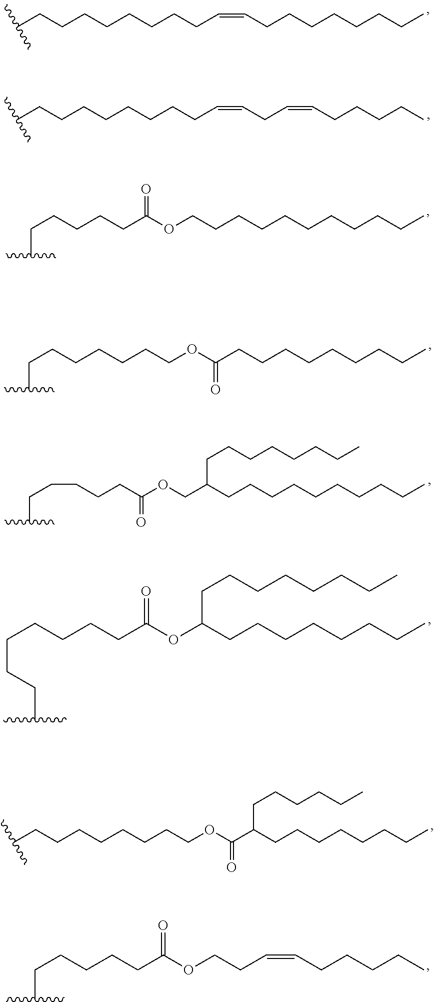

In some aspects, the present invention provides a cationic lipid represented by the structure of formula (IV):

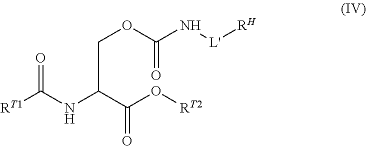

or a salt, hydrate, solvate, polymorph, optical isomer, geometrical isomer, enantiomer, diastereomer, tautomer, isotope labeled compound or mixtures thereof,
wherein L′ is —(CH₂)ₙ—, and wherein n=1, 2 or 3, preferably, n=2;
R^{H} is —N(CH₃)₂, —N(C₂H₅)₂,

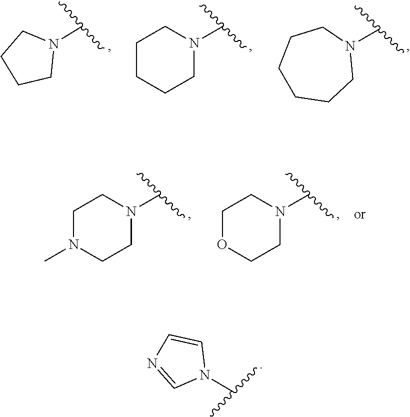

preferably, R^{H} is —N(CH₃)₂,

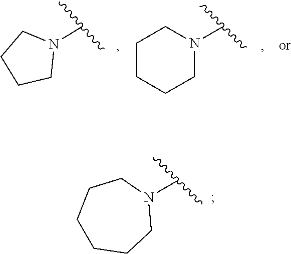 [SMILES: *N1CCCC1 ; *N1CCCCC1 ; *N1CCCCCC1]

R^{T1} and R^{T2} are each independently selected from the group consisting of:

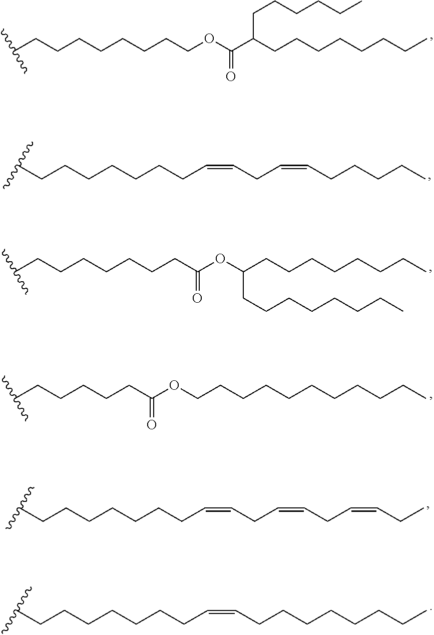 [SMILES: *CCCCCCCCOC(=O)C(CCCCCC)CCCCCCCC ; *CCCCCCC/C=C\C/C=C\CCCCC ; *CCCCCCCC(=O)OC(CCCCCCCC)CCCCCCCC ; *CCCCCC(=O)OCCCCCCCCCCC ; *CCCCCCC/C=C\C/C=C\C/C=C\CC ; *CCCCCCC/C=C\CCCCCCCC]

In some aspects, the present invention provides a cationic lipid represented by the structure of formula (V):

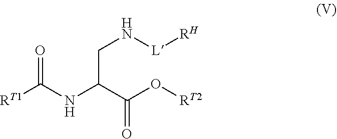

or a salt, hydrate, solvate, polymorph, optical isomer, geometrical isomer, enantiomer, diastereomer, tautomer, isotope labeled compound or mixtures thereof,
wherein L′ is —(CH₂)ₙ—, wherein n=1, 2 or 3, preferably, n=2;
R^{H} is —N(CH₃)₂, —N(C₂H₅)₂,

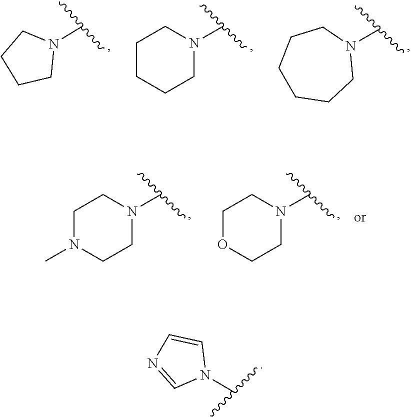

preferably, R^{H} is —N(CH₃)₂,

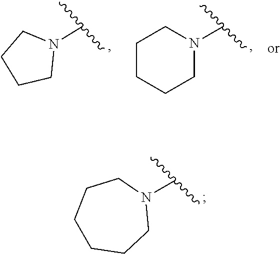 [SMILES: *N1CCCC1 ; *N1CCCCC1 ; *N1CCCCCC1]

R^{T1} and R^{T2} are each independently selected from the group consisting of:

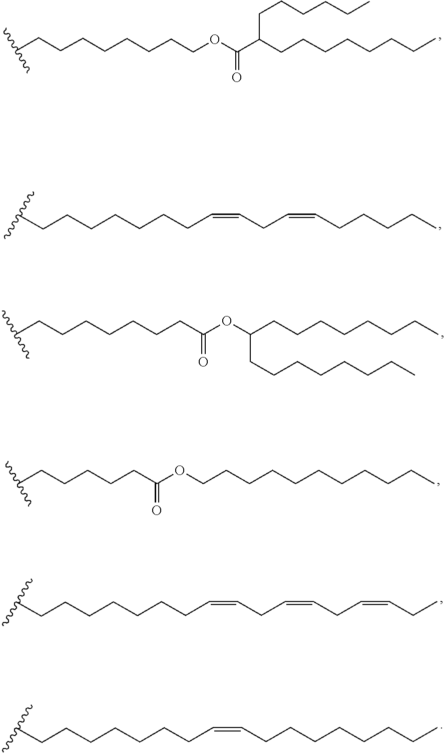 [SMILES: *CCCCCCCCOC(=O)C(CCCCCC)CCCCCCCC ; *CCCCCCC/C=C\C/C=C\CCCCC ; *CCCCCCCC(=O)OC(CCCCCCCC)CCCCCCCC ; *CCCCCC(=O)OCCCCCCCCCCC ; *CCCCCCC/C=C\C/C=C\C/C=C\CC ; *CCCCCCC/C=C\CCCCCCCC]

In some aspects, the present invention provides an intermediate compound represented by the structure of formula (VI):

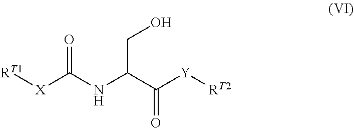

wherein
each of the groups is as defined herein.

In some aspects, the present invention provides a method of preparing the cationic lipid of the present invention, comprising a step of reacting a compound of formula (VI) with a compound of formula (VII), to yield the cationic lipid of formula (II):

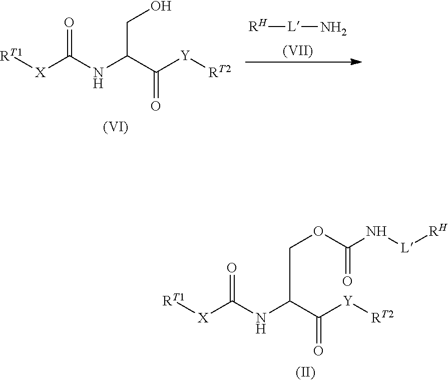

wherein each of the groups is as defined herein; and
the reaction is conducted in the presence of CDI.

In some aspects, the present invention provides a method of preparing the cationic lipid of the present invention, comprising a step of reacting a compound of formula (VI) with a compound of formula (VII), to yield the cationic lipid of formula (III):

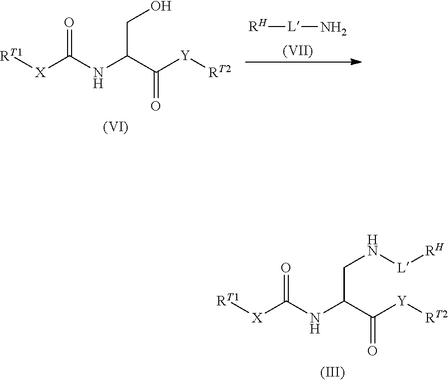

wherein each of the groups is as defined herein; and
the reaction is conducted in the presence of TsCl.

In some aspects, the present invention provides a nanoparticle composition, comprising a cationic lipid of the invention.

In some aspects, the present invention provides a nanoparticle composition, further comprising one or more selected from the group of a phospholipid, a PEG lipid and a structural lipid. In some aspects, the present invention provides a nanoparticle composition, further comprising a phospholipid, a PEG lipid and a structural lipid.

In some aspects, the present invention provides a pharmaceutical composition comprising a nanoparticle composition according to the present invention and a pharmaceutically acceptable carrier.

In some aspects, the present invention provides a method of delivering a therapeutic and/or prophylactic nucleic acid molecule to a cell, including the step of administering to a subject (i) the nanoparticle composition of the invention and (ii) a therapeutic and/or prophylactic nucleic acid molecule, in which administering involves contacting the cell with the nanoparticle composition, whereby the therapeutic and/or prophylactic nucleic acid molecule is delivered to the cell. In embodiment, the therapeutic and/or prophylactic nucleic acid is encapsulated in the nanoparticle composition of the present invention. In embodiment, the therapeutic and/or prophylactic nucleic acid is combined with the nanoparticle composition of the present invention.

In some aspects, the present invention provides a method of delivering a protein-coding nucleic acid molecule to a cell, including the step of administering to a subject (i) the nanoparticle composition of the invention and (ii) a protein-coding nucleic acid molecule, in which administering involves contacting the cell with the nanoparticle composition of the present invention, whereby the nucleic acid is delivered to the cell. In embodiment, the protein-coding nucleic acid is encapsulated in the nanoparticle composition of the present invention. In embodiment, the protein-coding nucleic acid is combined with the nanoparticle composition of the present invention.

In some aspects, the present invention provides a method of producing a polypeptide of interest in a cell, including the step of contacting the cell with a nanoparticle composition of the invention and (ii) a nucleic acid molecule encoding the polypeptide of interest, whereby the nucleic acid molecule is capable of being translated in the cell to produce the polypeptide. In embodiments, the nucleic acid molecule is an mRNA molecule, a siRNA molecule, or a circular RNA molecule. In embodiments, the nucleic acid molecule is a linear RNA. In embodiments, the nucleic acid molecule is a circular RNA.

In some aspects, the present invention provides a nanoparticle composition for use in the manufacture of a medicament for the treatment of a disease or disorder in a mammal in need thereof, wherein the nanoparticle composition includes (i) a lipid component including a phospholipid, a PEG lipid, a structural lipid, and a cationic lipid of the invention and (ii) a therapeutic and/or prophylactic nucleic acid molecule. In embodiments, the nucleic acid molecule is an mRNA, a siRNA or a circular RNA. In embodiments, the nucleic acid molecule is a linear RNA. In embodiments, the nucleic acid molecule is a circular RNA.

In some aspects, the present invention provides a nanoparticle composition for use in the manufacture of a medicament for the treatment of a disease or disorder in a mammal in need thereof, wherein the nanoparticle composition includes (i) a cationic lipid of the invention and (ii) a nucleic acid molecule encoding a therapeutic and/or prophylactic protein. In embodiments, the nucleic acid molecule is an mRNA, a siRNA or a circular RNA. In embodiments, the nucleic acid molecule is a linear RNA. In embodiments, the nucleic acid molecule is a circular RNA.

In some aspects, the present invention provides use of the cationic lipid of the invention, for the manufacture of a nanoparticle composition.

In some aspects, the present invention provides use of the cationic lipid of the invention or use of the nanoparticle composition of the invention for the manufacture of a medicament for the treatment of a disease or disorder in a subject in need thereof.

In some aspects, the present invention provides a method of synthesizing a cationic lipid of Formula (I)-1, (I)-1-A, (I)-1-B, (I)-1-C, (I), (IA), (II), (III), (IV), or (V) and methods of making a nanoparticle composition including a lipid component comprising the cationic lipid of Formula (I)-1, (I)-1-A, (I)-1-B, (I)-1-C, (I), (IA), (II), (III), (IV), or (V).

### DETAILED DESCRIPTION OF THE PRESENT INVENTION

The present invention provides novel lipids and lipid nanoparticle compositions including a novel cationic lipid. The present invention also provides methods of providing a protein-coding nucleic acid molecule to a mammalian cell, specifically delivering said protein-coding nucleic acid molecule to a mammalian organ and producing a polypeptide of interest in a mammalian cell. The present invention also provides methods of delivering a therapeutic and/or prophylactic nucleic acid molecule to a mammalian cell, specifically delivering a therapeutic and/or prophylactic nucleic acid molecule to a mammalian organ, producing a polypeptide of interest in a mammalian cell, and treating a disease or disorder in a mammal in need thereof. For example, a method of producing a polypeptide of interest in a cell involves contacting a nanoparticle composition comprising a linear RNA, e.g. an mRNA, a siRNA, or a circular RNA with a mammalian cell, whereby the mRNA may be translated to produce the polypeptide of interest. A method of delivering a therapeutic and/or prophylactic nucleic acid molecule to a mammalian cell or organ may involve administration of a nanoparticle composition including the therapeutic and/or prophylactic nucleic acid molecule to a subject, in which the administration involves contacting the cell or organ with the composition, whereby the therapeutic and/or prophylactic nucleic acid molecule is delivered to the cell or organ.

As used herein, the term “alkyl” or “alkyl group” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms), which is optionally substituted. The term “C1-14 alkyl” means an optionally substituted linear or branched, saturated hydrocarbon including 1-14 carbon atoms. Unless otherwise specified, an alkyl group described herein refers to both unsubstituted and substituted alkyl groups.

As used herein, the term “alkenyl” or “alkenyl group” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond, which is optionally substituted. The term “C₂₋₁₄ alkenyl” means an optionally substituted linear or branched hydrocarbon including 2-14 carbon atoms and at least one carbon-carbon double bond. For example, an alkenyl group may include one, two, three, four, or more carbon-carbon double bonds. Unless otherwise specified, an alkenyl group described herein refers to both unsubstituted and substituted alkenyl groups.

As used herein, the term “alkynyl” or “alkynyl group” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one carbon-carbon triple bond, which is optionally substituted. The term “C₂₋₁₄ alkynyl” means an optionally substituted linear or branched hydrocarbon including 2-14 carbon atoms and at least one carbon-carbon triple bond. An alkynyl group may include one, two, three, four, or more carbon-carbon triple bonds. For example, an alkynyl group may include one or more carbon-carbon triple bonds. Unless otherwise specified, an alkynyl group described herein refers to both unsubstituted and substituted alkynyl groups.

As used herein, the term “carbocycle” or “carbocyclic group” means an optionally substituted mono- or multi-cyclic system including one or more rings of carbon atoms. Rings may be three, four, five, six, seven, eight, nine, ten, eleven, twelve, thirteen, fourteen, fifteen, sixteen, seventeen, eighteen, nineteen, or twenty membered rings. The term “C₃₋₆ carbocycle” means a carbocycle including a single ring having 3-6 carbon atoms. Carbocycles may include one or more carbon-carbon double or triple bonds and may be non-aromatic or aromatic (e.g., cycloalkyl or aryl groups). Examples of carbocycles include cyclopropyl, cyclopentyl, cyclohexyl, phenyl, naphthyl, and 1,2-dihydronaphthyl groups. The term “cycloalkyl” as used herein means a non-aromatic carbocycle and may or may not include any double or triple bond. Unless otherwise specified, carbocycles described herein refers to both unsubstituted and substituted carbocycle groups, i.e., optionally substituted carbocycles.

As used herein, the term “heterocycle” or “heterocyclic group” means an optionally substituted mono- or multi-cyclic system including one or more rings, where at least one ring includes at least one heteroatom. Heteroatoms may be, for example, nitrogen, oxygen, or sulfur atoms. Rings may be three, four, five, six, seven, eight, nine, ten, eleven, twelve, thirteen, or fourteen membered rings. Heterocycles may include one or more double or triple bonds and may be non-aromatic or aromatic (e.g., heterocycloalkyl or heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. The term “heterocycloalkyl” as used herein means a non-aromatic heterocycle and may or may not include any double or triple bond. Unless otherwise specified, heterocycles described herein refers to both unsubstituted and substituted heterocycle groups, i.e., optionally substituted heterocycles.

As used herein, alkyl, alkenyl, alkylene, alkenylene, and cyclyl (e.g., carbocyclyl and heterocyclyl) groups may be optionally substituted unless otherwise specified. Optional substituents may be selected from the group consisting of, but are not limited to, a halogen atom (e.g., a chloride, bromide, fluoride, or iodide group), a carboxylic acid (e.g., —C(O)OH), an alcohol (e.g., a hydroxyl, —OH), an ester (e.g., —C(O)OR or —OC(O)R), an aldehyde (e.g., —C(O)H), a carbonyl (e.g., —C(O)R, alternatively represented by C═O), an acyl halide (e.g., —C(O)X, in which X is a halide selected from bromide, fluoride, chloride, and iodide), a carbonate (e.g., —OC(O)OR), an alkoxy (e.g., —OR), an acetal (e.g., —C(OR)₂R. in which each OR are alkoxy groups that can be the same or different and R is an alkyl or alkenyl group), a phosphate, a thiol (e.g., —SH), a sulfoxide (e.g., —S(O)R), a sulfmic acid (e.g., —S(O)OH), a sulfonic acid (e.g., —S(O)₂OH), a thial (e.g., —C(S)H), a sulfate, a sulfonyl, an amide (e.g., —C(O)NR₂, or—N(R)C(O)R), an azido (e.g., —N₃), a nitro (e.g., —NO₂), a cyano (e.g., —CN), an isocyano (e.g., —NC), an acyloxy (e.g., —OC(O)R), an amino (e.g., —NR₂, —NRH, or —NH₂), a carbamoyl (e.g., —OC(O)NR₂, —OC(O)NRH, or —OC(O)NH₂), a sulfonamide (e.g., —S(O)₂NR₂, —S(O)₂NRH, —S(O)₂NH₂, —N(R)S(O)₂R, —N(H)S(O)₂R, —N(R)S(O)₂H, or —N(H)S(O)₂H), an alkyl group, an alkenyl group, and a cyclyl (e.g., carbocyclyl or heterocyclyl) group. In any of the preceding, R is an alkyl or alkenyl group, as defined herein. In some embodiments, the substituent groups themselves may be further substituted with, for example, one, two, three, four, five, or six substituents as defined herein. For example, a C₁₋₆ alkyl group may be further substituted with one, two, three, four, five, or six substituents as described herein.

As used herein, the term “compound” is meant to include all isomers and isotope labeled compounds of the structure depicted. “Isotope” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

Compounds of the present invention that contain nitrogens can be converted to N-oxides by treatment with an oxidizing agent (e.g., 3-chloroperoxy benzoic acid and/or hydrogen peroxides) to afford other compounds of the disclosure. Thus, all shown and claimed nitrogen-containing compounds are considered, when allowed by valency and structure, to include both the compound as shown and its N-oxide derivative. Furthermore, in other instances, the nitrogens in the compounds of the disclosure can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

As used herein, the term “contacting” means establishing a physical connection between two or more entities. For example, contacting a mammalian cell with a nanoparticle composition means that the mammalian cell and a nanoparticle are made to share a physical connection. Methods of contacting cells with external entities both in vivo and ex vivo are well known in the biological arts. For example, contacting a nanoparticle composition and a mammalian cell disposed within a mammal may be performed by varied routes of administration (e.g., intravenous, intramuscular, intradermal, and subcutaneous) and may involve varied amounts of nanoparticle compositions. Moreover, more than one mammalian cell may be contacted by a nanoparticle composition.

As used herein, the term “delivering” means providing an entity to a destination. For example, delivering a therapeutic and/or prophylactic nucleic acid molecule to a subject may involve administering a nanoparticle composition including the therapeutic and/or prophylactic nucleic acid molecule to the subject (e.g., by an intravenous, intramuscular, intradermal, or subcutaneous route). Administration of a nanoparticle composition to a mammal or mammalian cell may involve contacting one or more cells with the nanoparticle composition.

As used herein, the term “isomer” means any geometric isomer, tautomer, zwitterion, stereoisomer, enantiomer, or diastereomer of a compound. Compounds may include one or more chiral centers and/or double bonds and may thus exist as stereoisomers, such as double bond isomers (i.e., geometric E/Z isomers) or diastereomers (e.g., enantiomers (i.e., (+) or (−)) or cis/trans isomers). The present disclosure encompasses any and all isomers of the compounds described herein, including stereomerically pure forms (e.g., geometrically pure,
enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerization is called tautomerism.

It is to be understood that the compounds of the present invention may be depicted as different tautomers. It should also be understood that when compounds have tautomeric forms, all tautomeric forms are intended to be included in the scope of the disclosure, and the naming of the compounds does not exclude any tautomer form.

As used herein, a “lipid component” is that component of a nanoparticle composition that includes one or more lipids. For example, the lipid component may include one or more cationic/ionizable, PEGylated, structural, or other lipids, such as phospholipids.

As used herein, “methods of administration” may include intravenous, intramuscular, intradermal, subcutaneous, or other methods of delivering a composition to a subject. A method of administration may be selected to target delivery (e.g., to specifically deliver) to a specific region or system of a body.

As used herein, a “nanoparticle composition” is a composition comprising one or more lipids. Nanoparticle compositions are typically sized on the order of micrometers or smaller and may include a lipid bilayer. Nanoparticle compositions encompass lipid nanoparticles (LNPs), liposomes (e.g., lipid vesicles), and lipoplexes. For example, a nanoparticle composition may be a liposome having a lipid bilayer with a diameter of 500 nm or less.

In the present specification, the structural formula of the compound represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. In addition, a crystal polymorphism may be present for the compounds represented by the formula. It is noted that any crystal form, crystal form mixture, or anhydride or hydrate thereof is included in the scope of the present disclosure.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

Compositions may also include salts of one or more compounds. Salts may be pharmaceutically acceptable salts. As used herein, “pharmaceutically acceptable salts” refers to derivatives of the disclosed compounds wherein the parent compound is altered by converting an existing acid or base moiety to its salt form (e.g., by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17* ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al, Journal of Pharmaceutical Science, 66, 1-19 (1977), each of which is incorporated herein by reference in its entirety.

As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

As used herein, the lipid component of a nanoparticle composition may include one or more PEG or PEG-modified lipids. Such species may be alternately referred to as PEGylated lipids. A PEG lipid is a lipid modified with polyethylene glycol. A PEG lipid may be selected from the non-limiting group consisting of PEG-modified phosphatidylethanolamines, PEG-modified phosphatidic acids, PEG-modified ceramides (PEG-CER), PEG-modified dialkylamines, PEG-modified diacylglycerols (PEG-DEG), PEG-modified dialkylglycerols, and mixtures thereof.

For example, a PEG lipid may be PEG-c-DOMG, PEG-DMG, PEG-DLPE, PEG-DMPE, PEG-DPPC, or a PEG-DSPE lipid.

As used herein, the lipid component of a nanoparticle composition may include one or more structural lipids. Structural lipids can be selected from the group consisting of, but are not limited to, cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the structural lipid includes cholesterol and a corticosteroid (such as prednisolone, dexamethasone, prednisone, and hydrocortisone), or a combination thereof.

As used herein, the lipid component of a nanoparticle composition may include one or more phospholipids, such as one or more (poly)unsaturated lipids. Phospholipids may assemble into one or more lipid bilayers. In general, phospholipids may include a phospholipid moiety and one or more fatty acid moieties. For example, a phospholipid moiety may be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated.

For example, a phospholipid may be functionalized with or cross-linked to one or more alkynes (e.g., an alkenyl group in which one or more double bonds is replaced with a triple bond).

As used herein, the term “polypeptide” or “polypeptide of interest” refers to a polymer of amino acid residues typically joined by peptide bonds that can be produced naturally (e.g., isolated or purified) or synthetically.

As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA), a small interference RNA (siRNA), or a circular RNA (cRNA). Translation of an mRNA or a circular RNA encoding a particular polypeptide, for example, in vivo translation thereof inside a mammalian cell, may produce the encoded polypeptide.

In some aspects, the present invention provides a cationic lipid represented by the structure of formula (I)-1:

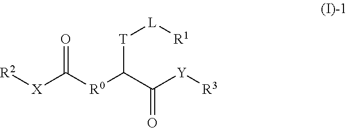

or a salt, hydrate, solvate, polymorph, optical isomer, geometrical isomer, enantiomer, diastereomer, tautomer, isotope labeled compound or mixtures thereof,
wherein:
L, X and Y are each independently selected from the group consisting of a direct bond, —O—, —C(═O)—, —OC(═O)—, —C(═O)O—, —OC(═O)O—, —C(═O)S—, —O—C(═O)S—, —NH—, —NHC(═O)—, —O—C(═O)—NH—, —NH—C(═O)—O—, —NH—C(═O)—NH—, —NH—C(═O)—S—, —NH(S=O)—, —NHS(═O)₂—, —S—, —S(═O)—, —S(═O)₂—, —W—W′—, —C₁₋₆ alkylene-, —C₂₋₆ alkenylene-, —W—C₁₋₆ alkylene-, —W—C₂₋₆ alkenylene-, —C₁₋₆ alkylene-W—, —C₂₋₆ alkenylene-W—, —W—W′—C₁₋₆ alkylene-, —C₁₋₆ alkylene-W—W′—, —W—W′—C₁₋₆ alkenylene-, —C₂₋₆ alkenylene-W—W′—, —W—C₁₋₆ alkylene-W′— and —W—C₂₋₆ alkenylene-W′—, wherein the alkylene and alkenylene are optionally further interrupted by one or more W;
W and W′, at each occurrence, are each independently selected from the group consisting of —O—, —C(═O)—, —OC(═O)—, —C(═O)O—, —OC(═O)O—, —C(═O)S—, —O—C(═O)S—, —NH—, —NHC(═O)—, —O—C(═O)—NH—, —NH—C(═O)—O—, —NH—C(═O)—NH—, —NH—C(═O)—S—, —NH(S=O)—, —NHS(═O)₂—, —S—, —S(═O)—, —S(═O)₂—, —C₁₋₆ alkylene-, and —C₂₋₆ alkenylene-;
T is —(CH₂)ₘ— or

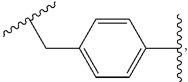 [SMILES: *Cc1ccc(*)cc1]

and m is 1, 2, 3 or 4;
R⁰ is selected from O and NH, provided that when R⁰ is O, then L must comprise a nitrogen atom; when R⁰ is NH, then L must comprise an oxygen atom;
R¹, R² and R³ are each independently selected from the group consisting of R^{H}, R^{T1} and R^{T2}, provided that one of R¹, R² and R³ is R^{H};
R^{H} is selected from the group consisting of:
   (a) NR⁴R⁵ wherein R⁴ and R⁵ are each independently H, C₁-C₁₀ alkyl (e.g., C₁-C₆ alkyl), —NH₂, halogen, —OH, a 3- to 10-membered cyclic ring or C₆₋₁₂ aralkyl, wherein the C₁-C₁₀ alkyl (e.g., C₁-C₆ alkyl), 3- to 10-membered cyclic ring or C₆₋₁₂ aralkyl is optionally substituted with one or more substituents selected from the group consisting of —NH₂, halogen, —OH, —C₁₋₆ alkyl and a 3- to 4-membered cyclic ring; or R⁴ and R⁵ together with the nitrogen to which they are attached form a 4- to 10-membered heterocyclic ring or 5- to 10-membered heteroaromatic ring, optionally containing one or more additional heteroatoms selected from the group consisting of O, N and S;
   (b) the side chain of a natural or unnatural amino acid;
   (c) a 3- to 10-membered cyclic ring, for example a C₆₋₁₂ aromatic ring, a 3- to 10-membered heterocyclic ring, a 5- to 10-membered heteroaromatic ring containing one or more heteroatoms selected from the group consisting of O, N and S, or a fused ring (e.g., a 4- to 10-membered fused ring), wherein the above cyclic ring is optionally substituted with one or more substituents selected from the group consisting of —NH₂, halogen, —OH, —C₁₋₆ alkyl, —C₁₋₆ alkylene-OH, and a 3- to 4-membered cyclic ring; and
   (d) —OH, or —C₁₋₁₀ alkyl optionally substituted with one or more substituents selected from the group consisting of —NH₂, halogen, —OH, —C₁₋₆ alkyl and a 3- to 4-membered cyclic ring;
preferably, R^{H} is selected from the group consisting of:
   (a) NR⁴R⁵ wherein R⁴ and R⁵ are each independently H, C₁-C₆ alkyl or C₆₋₁₂ aralkyl optionally substituted with —NH₂; or R⁴ and R⁵ together with the nitrogen to which they are attached form a 4- to 10-membered heterocyclic ring or 5- to 10-membered heteroaromatic ring, optionally containing one or more additional heteroatoms selected from the group consisting of O, N and S;
   (b) the side chain of a natural or unnatural amino acid; and
   (c) a 3- to 10-membered heterocyclic ring or 5- to 10-membered heteroaromatic ring containing one or more heteroatoms selected from the group consisting of O, N and S;
   (d) —OH or —C₁₋₁₀ alkyl optionally substituted with one or more substituents selected from the group consisting of —NH₂, halogen, —OH, —C₁₋₆ alkyl and a 3- to 4-membered cyclic ring;
R^{T1} and R^{T2} are each independently selected from the group consisting of:
   (a) C₁₀-C₂₂ alkyl;
   (b) C₁₀-C₂₂ alkenyl;
   (c) C₁₀-C₂₂ alkynyl;
   (d) C₄-C₁₅ alkylene-Z—C₄-C₂₂ alkyl; and
   (e) C₄-C₁₅ alkylene-Z—C₄-C₂₂ alkenyl;
   Z is —O—C(═O)—, —C(═O)—O— or —O—.

In some aspects, the present invention provides a cationic lipid represented by the structure of formula (I):

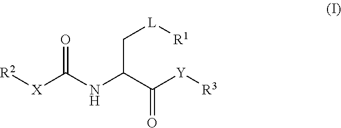

or a salt, hydrate, solvate, polymorph, optical isomer, geometrical isomer, enantiomer, diastereomer, tautomer, isotope labeled compound or mixtures thereof,
wherein:
L, X and Y are each independently selected from the group consisting of a direct bond, —O—, —C(═O)—, —OC(═O)—, —C(═O)O—, —NH—, —NHC(═O)—, —O—C(═O)—NH—, —NH—C(═O)—O—, —NH(S=O)—, —NHS(═O)₂—, —S—, —S(═O)—, —S(═O)₂—, —W—W′—, —C₁₋₆ alkylene-, —C₂₋₆ alkenylene-, —W—C₁₋₆ alkylene-, —W—C₂₋₆ alkenylene-, —C₁₋₆ alkylene-W—, —C₂₋₆ alkenylene-W—, —W—W′—C₁₋₆ alkylene-, —C₁₋₆ alkylene-W—W′—, —W—W′—C₁₋₆ alkenylene-, —C₂₋₆ alkenylene-W—W′—, —W—C₁₋₆ alkylene-W′— and —W—C₂₋₆ alkenylene-W′—, wherein the alkylene and alkenylene are optionally further interrupted by one or more W;
W and W′, at each occurrence, are each independently selected from the group consisting of —O—, —C(═O)—, —OC(═O)—, —C(═O)O—, —NH—, —NHC(═O)—, —O—C(═O)—NH—, —NH—C(═O)—O—, —NH(S=O)—, —NHS(═O)₂—, —S—, —S(═O)—, —S(═O)₂—, —C₁₋₆ alkylene-, and —C₂₋₆ alkenylene-;
R¹, R² and R³ are each independently selected from the group consisting of R^{H}, R^{T1} and R^{T2}, provided that one of R¹, R² and R³ is R^{H};
R^{H} is selected from the group consisting of:
   (a) NR⁴R⁵ wherein R⁴ and R⁵ are each independently H, C₁-C₁₀ alkyl (e.g., C₁-C₆ alkyl), —NH₂, halogen, —OH, a 3- to 10-membered cyclic ring or C₆₋₁₂ aralkyl, wherein the C₁-C₁₀ alkyl (e.g., C₁-C₆ alkyl), 3- to 10-membered cyclic ring or C₆₋₁₂ aralkyl is optionally substituted with one or more substituents selected from the group consisting of —NH₂, halogen, —OH, —C₁₋₆ alkyl and a 3- to 4-membered cyclic ring; or R⁴ and R⁵ together with the nitrogen to which they are attached form a 4- to 10-membered heterocyclic ring or 5- to 10-membered heteroaromatic ring, optionally containing one or more additional heteroatoms selected from the group consisting of O, N and S;
   (b) the side chain of a natural or unnatural amino acid;
   (c) a 3- to 10-membered cyclic ring, for example a C₆₋₁₂ aromatic ring, a 3- to 10-membered heterocyclic ring, a 5- to 10-membered heteroaromatic ring containing one or more heteroatoms selected from the group consisting of O, N and S, or a fused ring (e.g., a 4- to 10-membered fused ring), wherein the above cyclic ring is optionally substituted with one or more substituents selected from the group consisting of —NH₂, halogen, —OH, —C₁₋₆ alkyl, —C₁₋₆ alkylene-OH, and a 3- to 4-membered cyclic ring; and
   (d) —OH, or —C₁₋₁₀ alkyl optionally substituted with one or more substituents selected from the group consisting of —NH₂, halogen, —OH, —C₁₋₆ alkyl and a 3- to 4-membered cyclic ring;
preferably, R^{H} is selected from the group consisting of:
   (a) NR⁴R⁵ wherein R⁴ and R⁵ are each independently H, C₁-C₆ alkyl or C₆₋₁₂ aralkyl optionally substituted with —NH₂; or R⁴ and R⁵ together with the nitrogen to which they are attached form a 4- to 10-membered heterocyclic ring or 5- to 10-membered heteroaromatic ring, optionally containing one or more additional heteroatoms selected from the group consisting of O, N and S;
   (b) the side chain of a natural or unnatural amino acid; and
   (c) a 3- to 10-membered heterocyclic ring or 5- to 10-membered heteroaromatic ring containing one or more heteroatoms selected from the group consisting of O, N and S;
   (d) —OH or —C₁₋₁₀ alkyl optionally substituted with one or more substituents selected from the group consisting of —NH₂, halogen, —OH, —C₁₋₆ alkyl and a 3- to 4-membered cyclic ring;
R^{T1} and R^{T2} are each independently selected from the group consisting of:
   (a) C₁₀-C₂₂ alkyl;
   (b) C₁₀-C₂₂ alkenyl;
   (c) C₁₀-C₂₂ alkynyl;
   (d) C₄-C₁₅ alkylene-Z—C₄-C₂₂ alkyl; and
   (e) C₄-C₁₅ alkylene-Z—C₄-C₂₂ alkenyl;
   Z is —O—C(═O)—, —C(═O)—O— or —O—.

In some aspects, the cationic lipid of formula (I) has the structure of formula (I)-isomer A or (I)-isomer B:

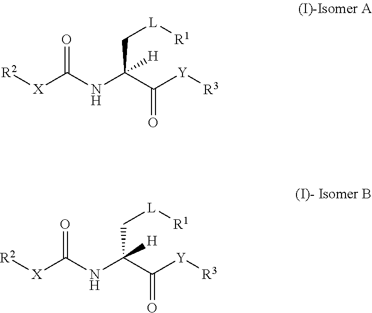

In some aspects, the present invention provides a cationic lipid, or a salt, hydrate, solvate, polymorph, optical isomer, geometrical isomer, enantiomer, diastereomer, tautomer, isotope labeled compound or mixtures thereof, wherein the cationic lipid is represented by the structure of formula (IA):

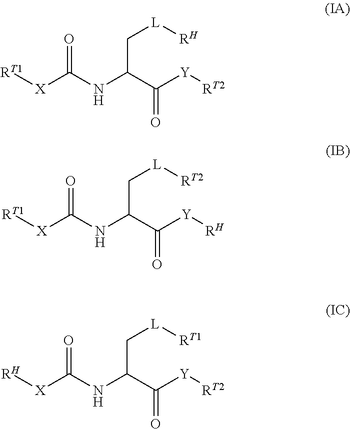

wherein each group is as defined herein.

In some aspects, the present invention provides a cationic lipid, or a salt, hydrate, solvate, polymorph, optical isomer, geometrical isomer, enantiomer, diastereomer, tautomer, isotope labeled compound or mixtures thereof, wherein the cationic lipid is represented by the structure of formula (II) or (III):

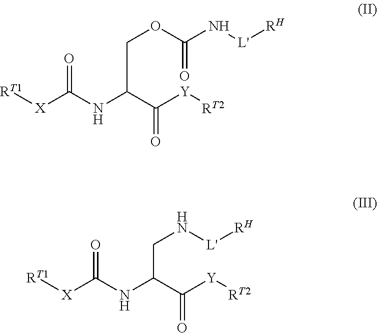

wherein:
L′ is —C₁₋₆ alkylene- or —C₂₋₆ alkenylene-;
R^{H} is selected from the group consisting of:
   (a) NR⁴R⁵ wherein R⁴ and R are each independently H, C₁-C₁₀ alkyl (e.g., C₁-C₆ alkyl), —NH₂, halogen, —OH, a 3- to 10-membered cyclic ring or C₆₋₁₂ aralkyl, wherein the C₁-C₁₀ alkyl (e.g., C₁-C₆ alkyl), 3- to 10-membered cyclic ring or C₆₋₁₂ aralkyl is optionally substituted with one or more substituents selected from the group consisting of —NH₂, halogen, —OH, —C₁₋₆ alkyl and a 3- to 4-membered cyclic ring; or R⁴ and R⁵ together with the nitrogen to which they are attached form a 4- to 10-membered heterocyclic ring or 5- to 10-membered heteroaromatic ring, optionally containing one or more additional heteroatoms selected from the group consisting of O, N and S;
   (b) the side chain of a natural or unnatural amino acid;
   (c) a 3- to 10-membered cyclic ring, for example a C₆₋₁₂ aromatic ring, a 3- to 10-membered heterocyclic ring, a 5- to 10-membered heteroaromatic ring containing one or more heteroatoms selected from the group consisting of O, N and S, or a fused ring (e.g., a 4- to 10-membered fused ring), wherein the above cyclic ring is optionally substituted with one or more substituents selected from the group consisting of —NH₂, halogen, —OH, —C₁₋₆ alkyl, —C₁₋₆ alkylene-OH, and a 3- to 4-membered cyclic ring; and
   (d) —OH, or —C₁₋₁₀ alkyl optionally substituted with one or more substituents selected from the group consisting of —NH₂, halogen, —OH, —C₁₋₆ alkyl and a 3- to 4-membered cyclic ring;
preferably, R^{H} is selected from the group consisting of:
   (a) NR⁴R⁵ wherein R⁴ and R⁵ are each independently H, C₁-C₆ alkyl or C₆₋₁₂ aralkyl optionally substituted with —NH₂; or R⁴ and R⁵ together with the nitrogen to which they are attached form a 4- to 10-membered heterocyclic ring or 5- to 10-membered heteroaromatic ring, optionally containing one or more additional heteroatoms selected from the group consisting of O, N and S;
   (b) the side chain of a natural or unnatural amino acid; and
   (c) a 3- to 10-membered heterocyclic ring or 5- to 10-membered heteroaromatic ring containing one or more heteroatoms selected from the group consisting of O, N and S; and
   (d) —OH, or —C₁₋₁₀ alkyl optionally substituted with one or more substituents selected from the group consisting of —NH₂, halogen, —OH, —C₁₋₆ alkyl and a 3- to 4-membered cyclic ring
R^{T1} and R^{T2} are each independently selected from the group consisting of:
   (a) C₁₀-C₂₂ alkyl;
   (b) C₁₀-C₂₂ alkenyl;
   (c) C₁₀-C₂₂ alkynyl;
   (d) C₄-C₁₅ alkylene-Z—C₄-C₂₂ alkyl; and
   (e) C₄-C₁₅ alkylene-Z—C₄-C₂₂ alkenyl;
   Z is —O—C(═O)—, —C(═O)—O— or —O—.

In embodiments, the present invention provides a cationic lipid of formula (I)-1, (I)-1-A, (I)-1-B, (I)-1-C, (I), (IA), (IB) or (IC), or a salt, hydrate, solvate, polymorph, optical isomer, geometrical isomer, enantiomer, diastereomer, tautomer, isotope labeled compound or mixtures thereof, wherein L is selected from the group consisting of a direct bond, —W—C₁₋₆ alkylene-, —W—C₂₋₆ alkenylene-, —C₁₋₆ alkylene-W—, —C₂₋₆ alkenylene-W—, —W—W′—C₁₋₆ alkylene-, —C₁₋₆ alkylene-W—W′—, —W—W′—C₁₋₆ alkenylene-, —C₂₋₆ alkenylene-W—W′—, —W—C₁₋₆ alkylene-W′— and —W—C₂₋₆ alkenylene-W′—;
W and W′, at each occurrence, are each independently selected from the group consisting of —O—, —C(═O)—, —C(═O)O—, —NH—, —NHC(═O)—, —O—C(═O)—NH—, —NH—C(═O)—O—, —NH(S=O)—, —NHS(═O)₂—, —S—, —S(═O)—, —S(═O)₂—, —C₁₋₆ alkylene-, and —C₂₋₆ alkenylene-.

In embodiments, the present invention provides a cationic lipid of formula (I)-1, (I)-1-A, (I)-1-B, (I)-1-C, (I), (IA), (IB) or (IC), or a salt, hydrate, solvate, polymorph, optical isomer, geometrical isomer, enantiomer, diastereomer, tautomer, isotope labeled compound or mixtures thereof, wherein L is a direct bond, —W—C₁₋₆ alkylene-, —C₁₋₆ alkylene-W—, —W—W′—C₁₋₆ alkylene-, or —W—C₁₋₆ alkylene-W′—;
W and W′, at each occurrence, are each independently selected from the group consisting of —O—, —C(═O)—, —C(═O)O—, —NH—, —NHC(═O)—, —O—C(═O)—NH—, and —NH—C(═O)—O—.

In embodiments, the present invention provides a cationic lipid of formula (I)-1, (I)-1-A, (I)-1-B, (I)-1-C, (I), (IA), (IB) or (IC), or a salt, hydrate, solvate, polymorph, optical isomer, geometrical isomer, enantiomer, diastereomer, tautomer, isotope labeled compound or mixtures thereof, wherein L is -a direct bond, —C₁₋₆ alkylene-NH—, —NH—C₁₋₆ alkylene-, —O—C(═O)—NH—C₁₋₆ alkylene-, or —C₁₋₆ alkylene-NH—C(═O)—O—.

In embodiments, the present invention provides a cationic lipid of formula (I)-1, (I)-1-A, (I)-1-B, (I)-1-C, (I), (IA), (IB) or (IC), or a salt, hydrate, solvate, polymorph, optical isomer, geometrical isomer, enantiomer, diastereomer, tautomer, isotope labeled compound or mixtures thereof, wherein L is —(CH₂)ₙ—NH—, —NH—(CH₂)ₙ—, —O—C(═O)—NH—(CH₂)ₙ—, or —(CH₂)ₙ—NH—C(═O)—O—, wherein n=1, 2 or 3.

In embodiments, L is —NH—(CH₂)ₙ—.

In embodiments, L is —O—C(═O)—NH—(CH₂)ₙ—.

In embodiments, n=2.

In embodiments, the present invention provides a cationic lipid of formula (I)-1, (I)-1-A, (I)-1-B, (I)-1-C, (I), (IA), (IB), (IC), (II) or (III), or a salt, hydrate, solvate, polymorph, optical isomer, geometrical isomer, enantiomer, diastereomer, tautomer, isotope labeled compound or mixtures thereof, wherein X is a direct bond, —O—, —NH—, or —S—.

In embodiments, X is a direct bond.

In embodiments, X is —O—.

In embodiments, the present invention provides a cationic lipid of formula (I)-1, (I)-1-A, (I)-1-B, (I)-1-C, (I), (IA), (IB), (IC), (II) or (III), or a salt, hydrate, solvate, polymorph, optical isomer, geometrical isomer, enantiomer, diastereomer, tautomer, isotope labeled compound or mixtures thereof, wherein Y is a direct bond, —O—, —NH—, or —S—.

In embodiments, Y is —O—.

In embodiments, the present invention provides a cationic lipid of formula (I)-1, (I)-1-A, (I)-1-B, (I)-1-C, (I), (IA), (IB), (IC), (II) or (III), or a salt, hydrate, solvate, polymorph, optical isomer, geometrical isomer, enantiomer, diastereomer, tautomer, isotope labeled compound or mixtures thereof, wherein R^{H} is selected from the group consisting of:
(a) NR⁴R⁵, wherein R⁴ and R⁵ are each independently H, C₁-C₃ alkyl or C₆₋₁₂ aralkyl optionally substituted with —NH₂; or R⁴ and R together with the nitrogen to which they are attached form a 5- to 6-membered heterocyclic ring or 5- to 6-membered heteroaromatic ring, optionally containing one or more additional heteroatoms selected from the group consisting of O, N and S;
(b) a 5- to 6-membered heterocyclic ring or 5- to 6-membered heteroaromatic ring containing one or more heteroatoms selected from the group consisting of O, N and S, or a fused ring (e.g., a 4- to 10-membered fused ring); and
(c) —OH.

In embodiments, the present invention provides a cationic lipid of formula (I)-1, (I)-1-A, (I)-1-B, (I)-1-C, (I), (IA), (IB), (IC), (II) or (III), or a salt, hydrate, solvate, polymorph, optical isomer, geometrical isomer, enantiomer, diastereomer, tautomer, isotope labeled compound or mixtures thereof, wherein R^{H} is NR⁴R⁵, wherein R⁴ and R⁵ are each independently H, or C₁-C₃ alkyl; or R⁴ and R⁵ together with the nitrogen to which they are attached form a 5- to 6-membered heterocyclic ring or 5- to 6-membered heteroaromatic ring, optionally containing one or more additional heteroatoms selected from the group consisting of O, N and S.

In embodiments, the present invention provides a cationic lipid of formula (I)-1, (I)-1-A, (I)-1-B, (I)-1-C, (I), (IA), (IB), (IC), (II) or (III), wherein R^{H} is selected from the group consisting of —OH, —NH₂, —N(CH₃)₂, —N(C₂H₅)₂,

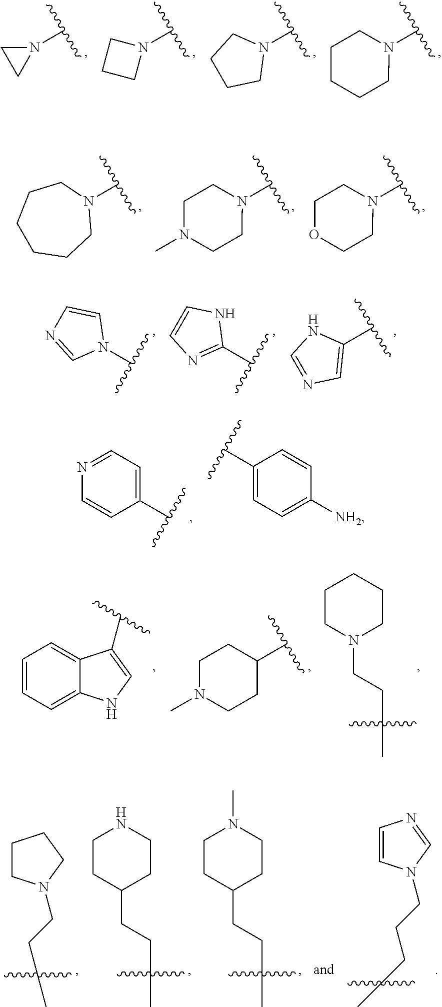

In embodiments, R^{H} is selected from the group consisting of —OH, —N(CH₃)₂, —N(C₂H₅)₂,

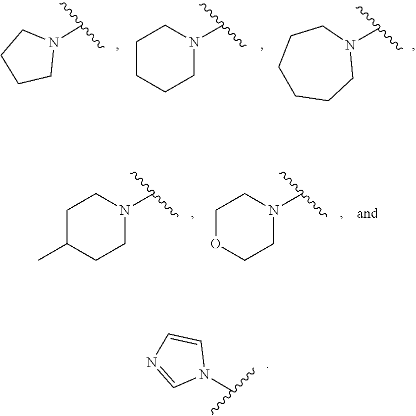

In embodiments, the present invention provides a cationic lipid of formula (I)-1, (I)-1-A, (I)-1-B, (I)-1-C, (I), (IA), (IB), (IC), (II) or (III), or a salt, hydrate, solvate, polymorph, optical isomer, geometrical isomer, enantiomer, diastereomer, tautomer, isotope labeled compound or mixtures thereof, wherein R^{T1} and R^{T2} are each independently selected from the group consisting of:
(a) C₁₀-C₂₂ alkyl, for example C₁₀, C₁₁, C₁₂, C₁₃, C₁₄, C₁₅, C₁₆, C₁₇, C₁₈, C₁₉ or C₂₀ alkyl;
(b) C₁₀-C₂₂ alkenyl, for example C₁₀, C₁₁, C₁₂, C₁₃, C₁₄, C₁₅, C₁₆, C₁₇, C₁₈, C₁₉ or C₂₀ alkenyl;
(d) C₄-C₁₅ alkylene-Z—C₄-C₂₂ alkyl; and
(e) C₄-C₁₅ alkylene-Z—C₄-C₂₂ alkenyl;
Z is —O—C(═O)—, —C(═O)—O— or —O—.

In embodiments, the present invention provides a cationic lipid of formula (I)-1, (I)-1-A, (I)-1-B, (I)-1-C, (I), (IA), (IB), (IC), (II) or (III), or a salt, hydrate, solvate, polymorph, optical isomer, geometrical isomer, enantiomer, diastereomer, tautomer, isotope labeled compound or mixtures thereof, wherein R^{T1} and R^{T2} are each independently selected from the group consisting of:
(a) C₁₀-C₂₂ alkyl, for example C₁₀, C₁₁, C₁₂, C₁₃, C₁₄, C₁₅, C₁₆, C₁₇, C₁₈, C₁₉ or C₂₀ alkyl;
(b) C₁₀-C₂₂ alkenyl, for example C₁₀, C₁₁, C₁₂, C₁₃, C₁₄, C₁₅, C₁₆, C₁₇, C₁₈, C₁₉ or C₂₀ alkenyl;
(c) Cₚ alkylene-Z—C_{q} alkyl; and
(d) Cₚ alkylene-Z—C_{q} alkenyl;
Z is —O—C(═O)—, —C(═O)—O— or —O—,
p is any integer between 4 to 15, for example 4, 5, 6, 7, 8, 9, 10, 11, 12, 13, 14 or 15,
q is any integer between 4 to 22, for example 4, 5, 6, 7, 8, 9, 10, 11, 12, 13, 14, 15, 16, 17, 18, 19, 20, 21 or 22.

In embodiments, the present invention provides a cationic lipid of formula (I)-1, (I)-1-A, (I)-1-B, (I)-1-C, (I), (IA), (IB), (IC), (II) or (III), or a salt, hydrate, solvate, polymorph, optical isomer, geometrical isomer, enantiomer, diastereomer, tautomer, isotope labeled compound or mixtures thereof, wherein R^{T1} and R^{T2} are each independently selected from the group consisting of:
(b) C₁₀-C₂₂ alkenyl, for example C₁₀, C₁₁, C₁₂, C₁₃, C₁₄, C₁₅, C₁₆, C₁₇, C₁₈, C₁₉ or C₂₀ alkenyl; and
(c) Cₚ alkylene-Z—C_{q} alkyl;
Z is —O—C(═O)— or —C(═O)—O—,
p is any integer between 4 to 15, for example 4, 5, 6, 7, 8, 9, 10, 11, 12, 13, 14 or 15,
q is any integer between 4 to 22, for example 4, 5, 6, 7, 8, 9, 10, 11, 12, 13, 14, 15, 16, 17, 18, 19, 20, 21 or 22.

In embodiments, the present invention provides a cationic lipid of formula (I)-1, (I)-1-A, (I)-1-B, (I)-1-C, (I), (IA), (IB), (IC), (II) or (III), or a salt, hydrate, solvate, polymorph, optical isomer, geometrical isomer, enantiomer, diastereomer, tautomer, isotope labeled compound or mixtures thereof, wherein R^{T1} and R^{T2} are each independently selected from the group consisting of:
(c) Cₚ alkylene-Z—C_{q} alkyl;
Z is —O—C(═O)— or —C(═O)—O—,
p is any integer between 4 to 15, for example 4, 5, 6, 7, 8, 9, 10, 11, 12, 13, 14 or 15,
q is any integer between 4 to 22, for example 4, 5, 6, 7, 8, 9, 10, 11, 12, 13, 14, 15, 16, 17, 18, 19, 20, 21 or 22,
Z in R^{T1} and R^{T2} are different.

In embodiments, the present invention provides a cationic lipid of formula (I)-1, (I)-1-A, (I)-1-B, (I)-1-C, (I), (IA), (IB), (IC), (II) or (III), or a salt, hydrate, solvate, polymorph, optical isomer, geometrical isomer, enantiomer, diastereomer, tautomer, isotope labeled compound or mixtures thereof, wherein R^{T1} and R^{T2} respectively are
(b) C₁₆-C₂₀ alkenyl, for example C₁₆, C₁₇, C₁₈, C₁₉ or C₂₀ alkenyl, and
(c) Cₚ alkylene-Z—C_{q} alkyl,
Z is —O—C(═O)— or —C(═O)—O—,
p is any integer between 4 to 15, for example 4, 5, 6, 7, 8, 9, 10, 11, 12, 13, 14 or 15,
q is any integer between 4 to 22, for example 4, 5, 6, 7, 8, 9, 10, 11, 12, 13, 14, 15, 16, 17, 18, 19, 20, 21 or 22.

In embodiments, the present invention provides a cationic lipid of formula (I)—I, (I)-1-A, (I)-1-B, (I)-1-C, (I), (IA), (IB), (IC), (II) or (III), or a salt, hydrate, solvate, polymorph, optical isomer, geometrical isomer, enantiomer, diastereomer, tautomer, isotope labeled compound or mixtures thereof, wherein R^{T1} and R^{T2} are
(c) Cₚ alkylene-Z—C_{q} alkyl,
Z is —O—C(═O)— or —C(═O)—O—,
p is any integer between 4 to 15, for example 4, 5, 6, 7, 8, 9, 10, 11, 12, 13, 14 or 15,
q is any integer between 4 to 22, for example 4, 5, 6, 7, 8, 9, 10, 11, 12, 13, 14, 15, 16, 17, 18, 19, 20, 21 or 22,
Z in R^{T1} and R^{T2} are the same, for example, Z in R^{T1} and R^{T2} is —O—C(═O)—.

In embodiments, the present invention provides a cationic lipid of formula (I)-1, (I)-1-A, (I)-1-B, (I)-1-C, (I), (IA), (IB), (IC), (II) or (III), or a salt, hydrate, solvate, polymorph, optical isomer, geometrical isomer, enantiomer, diastereomer, tautomer, isotope labeled compound or mixtures thereof, wherein R^{T1} and R^{T2} are each independently selected from the group consisting of:

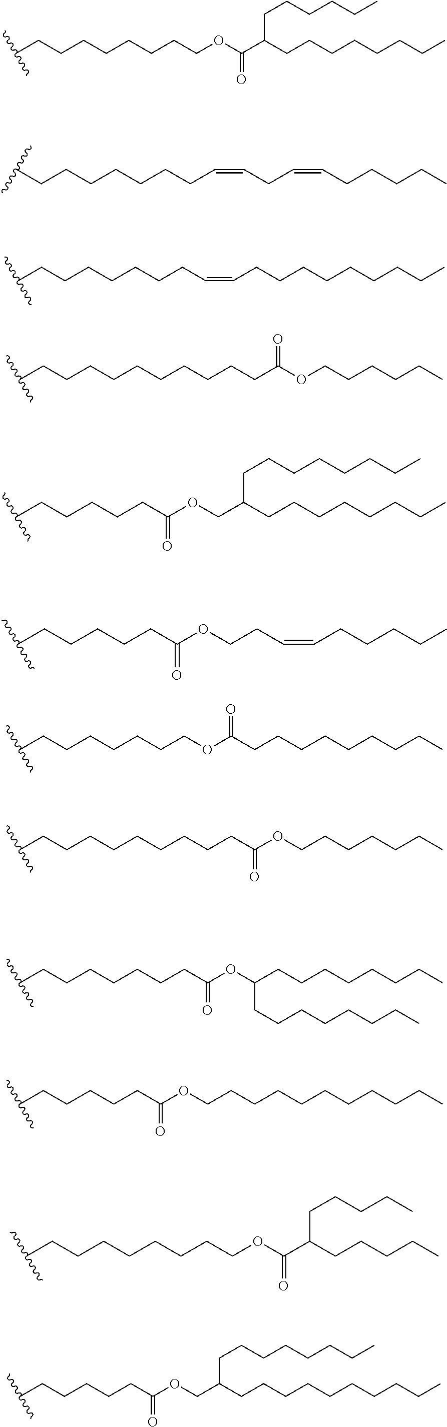

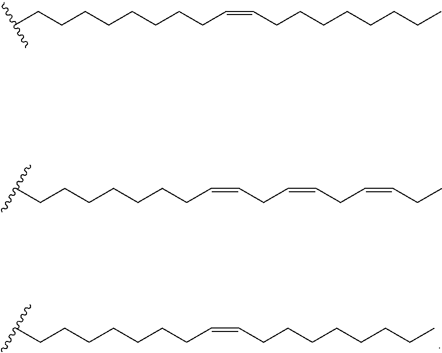 [SMILES: *CCCCCCCC/C=C\CCCCCCCC ; *CCCCCCC/C=C\C/C=C\C/C=C\CC ; *CCCCCCC/C=C\CCCCCCCC]

In embodiments, the present invention provides a cationic lipid of formula (I)-1, (I)-1-A, (I)-1-B, (I)-1-C, (I), (IA), (IB), (IC), (II) or (III), or a salt, hydrate, solvate, polymorph, optical isomer, geometrical isomer, enantiomer, diastereomer, tautomer, isotope labeled compound or mixtures thereof, wherein R^{T1} and R^{T2} are each independently selected from the group consisting of

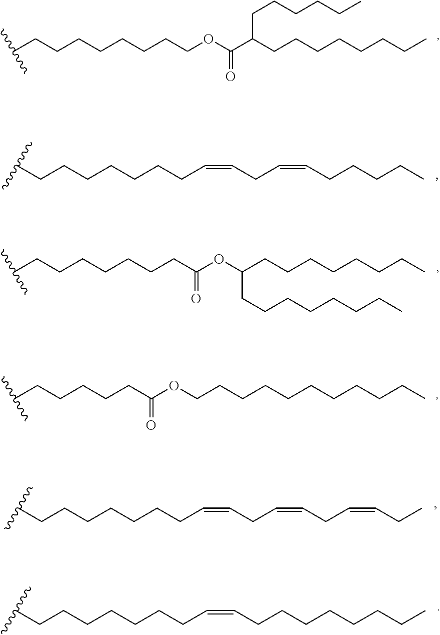 [SMILES: *CCCCCCCCOC(=O)C(CCCCCC)CCCCCCCC ; *CCCCCCC/C=C\C/C=C\CCCCC ; *CCCCCCCC(=O)OC(CCCCCCCC)CCCCCCCC ; *CCCCCC(=O)OCCCCCCCCCCC ; *CCCCCCC/C=C\C/C=C\C/C=C\CC ; *CCCCCCC/C=C\CCCCCCCC]

In embodiments, the present invention provides a cationic lipid of formula (I)-1, (I)-1-A, (I)-1-B, (I)-1-C, (I), (IA), (IB), (IC), (II) or (III), or a salt, hydrate, solvate, polymorph, optical isomer, geometrical isomer, enantiomer, diastereomer, tautomer, isotope labeled compound or mixtures thereof, wherein R^{T1} and R^{T2} are the same groups, and are

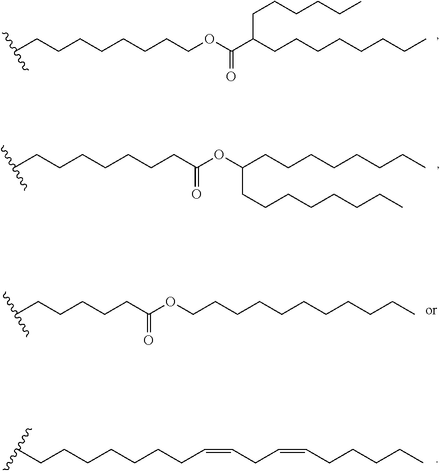 [SMILES: *CCCCCCCCOC(=O)C(CCCCCC)CCCCCCCC ; *CCCCCCCC(=O)OC(CCCCCCCC)CCCCCCCC ; *CCCCCC(=O)OCCCCCCCCCCC ; *CCCCCCC/C=C\C/C=C\CCCCC]

In embodiments, the present invention provides a cationic lipid of formula (I)-1, (I)-1-A, (I)-1-B, (I)-1-C, (I), (IA), (IB), (IC), (II) or (III), or a salt, hydrate, solvate, polymorph, optical isomer, geometrical isomer, enantiomer, diastereomer, tautomer, isotope labeled compound or mixtures thereof, wherein R^{T1} and R^{T2} are different groups and respectively are
wherein R^{T1} and R^{T2} are different groups and respectively are

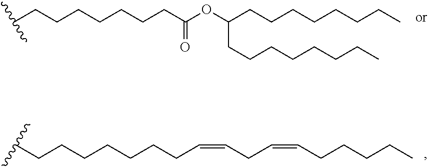 [SMILES: *CCCCCCCC(=O)OC(CCCCCCCC)CCCCCCCC ; *CCCCCCC/C=C\C/C=C\CCCCC]

or
wherein R^{T1} and R^{T2} are different groups and respectively are

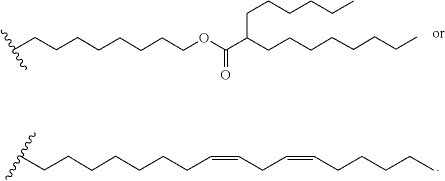 [SMILES: *CCCCCCCCOC(=O)C(CCCCCC)CCCCCCCC ; *CCCCCCC/C=C\C/C=C\CCCCC]

In some aspects, the present invention provides a cationic lipid represented by the structure of formula (II):

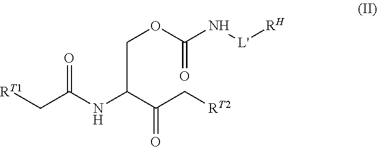

or a salt, hydrate, solvate, polymorph, optical isomer, geometrical isomer, enantiomer, diastereomer, tautomer, isotope labeled compound or mixtures thereof,
wherein
L′ is —(CH₂)ₙ—, and wherein n=1, 2 or 3, preferably, n=2;
R^{H} is —N(CH₃)₂, —N(C₂H₅)₂,

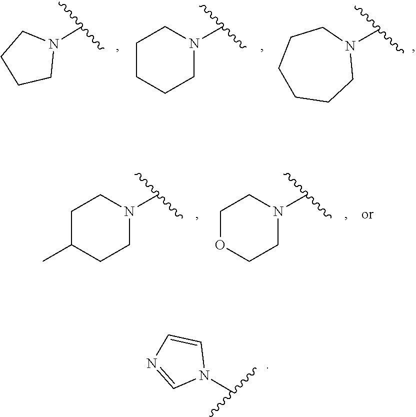

preferably, R^{H} is —N(CH₃)₂,

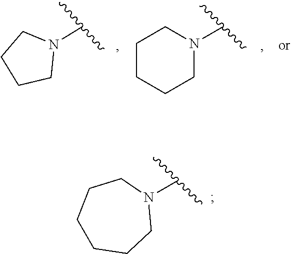 [SMILES: *N1CCCC1 ; *N1CCCCC1 ; *N1CCCCCC1]

### X is —O—;

Y is —O—; and
R^{T1} and R^{T2} are each independently selected from the group consisting of:

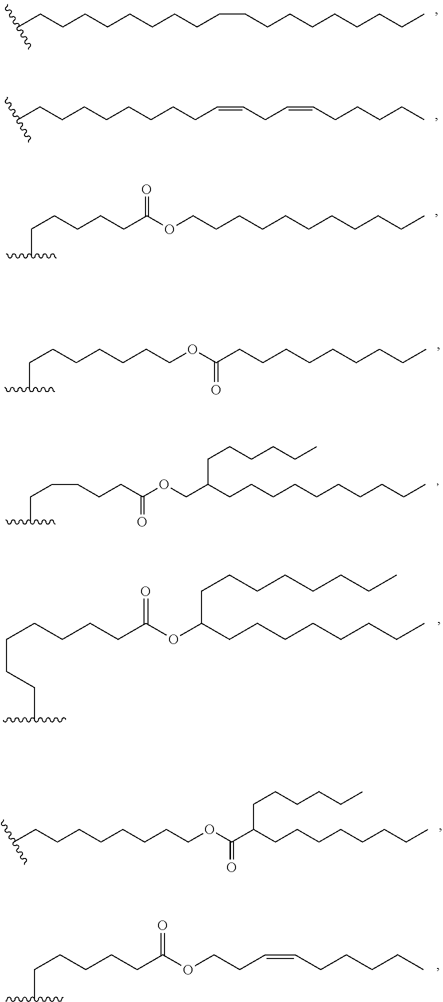

In some aspects, the present invention provides a cationic lipid represented by the structure of formula (IV):

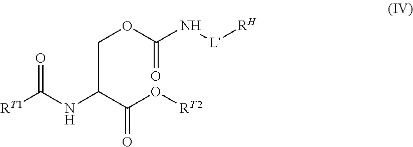

or a salt, hydrate, solvate, polymorph, optical isomer, geometrical isomer, enantiomer, diastereomer, tautomer, isotope labeled compound or mixtures thereof,
wherein L′ is —(CH₂)ₙ—, and wherein n=1, 2 or 3, preferably, n=2;
R^{H} is —N(CH₃)₂, —N(C₂H₅)₂,

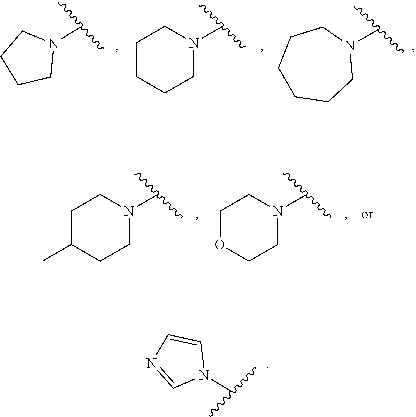

preferably, R^{H} is —N(CH₃)₂,

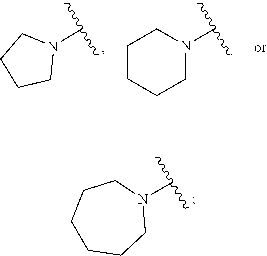 [SMILES: *N1CCCC1 ; *N1CCCCC1 ; *N1CCCCCC1]

R^{T1} and R^{T2} are each independently selected from the group consisting of:

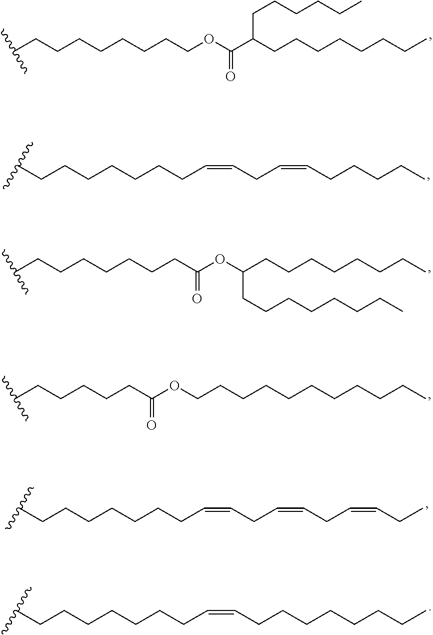 [SMILES: *CCCCCCCCOC(=O)C(CCCCCC)CCCCCCCC ; *CCCCCCC/C=C\C/C=C\CCCCC ; *CCCCCCCC(=O)OC(CCCCCCCC)CCCCCCCC ; *CCCCCC(=O)OCCCCCCCCCCC ; *CCCCCCC/C=C\C/C=C\C/C=C\CC ; *CCCCCCC/C=C\CCCCCCCC]

In some aspects, the present invention provides a cationic lipid represented by the structure of formula (V):

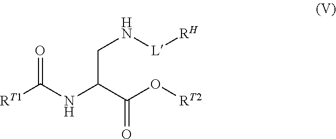

or a salt, hydrate, solvate, polymorph, optical isomer, geometrical isomer, enantiomer, diastereomer, tautomer, isotope labeled compound or mixtures thereof,
wherein L′ is —(CH₂)ₙ—, wherein n=1, 2 or 3, preferably, n=2;
R^{H} is —N(CH₃)₂,

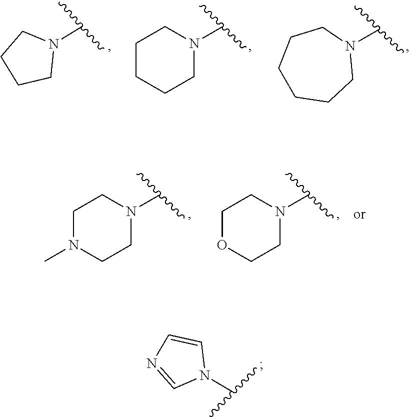

preferably; R^{H} is —N(CH₃)₂,

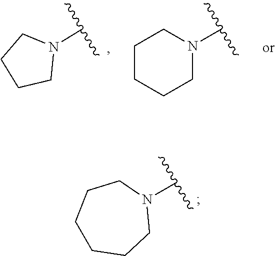 [SMILES: *N1CCCC1 ; *N1CCCCC1 ; *N1CCCCCC1]

R^{T1} and R^{T2} are each independently selected from the group consisting of

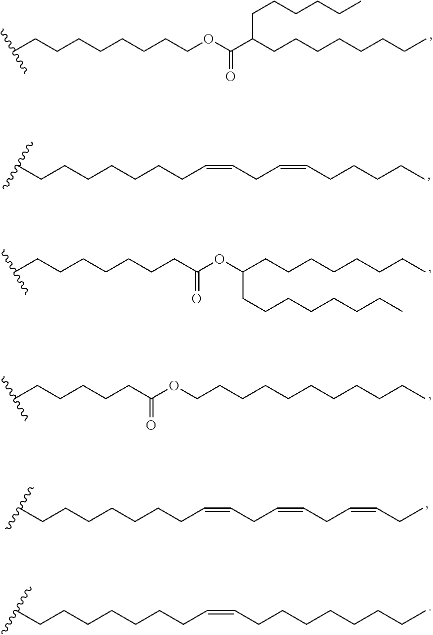 [SMILES: *CCCCCCCCOC(=O)C(CCCCCC)CCCCCCCC ; *CCCCCCC/C=C\C/C=C\CCCCC ; *CCCCCCCC(=O)OC(CCCCCCCC)CCCCCCCC ; *CCCCCC(=O)OCCCCCCCCCCC ; *CCCCCCC/C=C\C/C=C\C/C=C\CC ; *CCCCCCC/C=C\CCCCCCCC]

In some aspects, the present invention provides a cationic lipid represented by the structure of formula (I)-1-A:

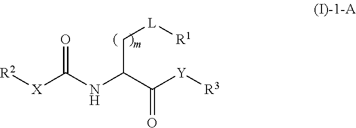

wherein:
L is —OC(═O)—, —OC(═O)O—, —OC(═O)—NH—, —OC(═O)S—, or —OC(═O)NH-L′-;
X is a direct bond, —O—, —NH—, —S— or —NH-L′-;
Y is —O—, —NH— or —NH-L′-;
L′ is —(CH₂)ₙ—, and wherein n=1, 2 or 3;
R¹, R² and R³ are each independently selected from the group consisting of R^{H}, R^{T1} and R^{T2}, provided that one of R¹, R² and R³ is R^{H};
m is 1, 2, 3 or 4.

In some aspects, the present invention provides a cationic lipid represented by the structure of formula (I)-1-B:

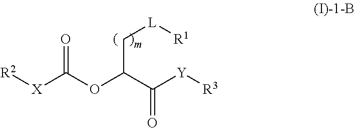

wherein:
L is —NHC(═O)—, —NHC(═O)—O—, —NHC(═O)—NH—, —NHC(═O)—S—, or —NHC(═O)NH-L′-;
X is a direct bond, —O—, —NH—, —S— or —NH-L′-;
Y is —O—, —NH— or —NH-L′-;
L′ is —(CH₂)ₙ—, and wherein n=1, 2 or 3;
R¹, R² and R³ are each independently selected from the group consisting of R^{H}, R^{T1} and R^{T2}, provided that one of R¹, R² and R³ is R^{H}.
m is 1, 2, 3 or 4.

In some aspects, the present invention provides a cationic lipid represented by the structure of formula (I)-1-C:

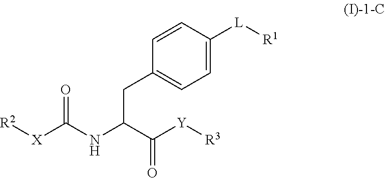

wherein:
L is —OC(═O)—, —OC(═O)O—, —OC(═O)—NH—, —OC(═O)S—, or —OC(═O)NH-L′-;
X is a direct bond, —O—, —NH—, —S— or —NH-L′-;
Y is —O—, —NH— or —NH-L′-;
L′ is —(CH₂)ₙ—, and wherein n=1, 2 or 3;
R¹, R² and R³ are each independently selected from the group consisting of R^{H}, R^{T1} and R^{T2}, provided that one of R¹, R² and R³ is R^{H};
m is 1, 2, 3 or 4.

Preferably, in formula (I)-1-A, formula (I)-1-B and formula (I)-1-C, m is 1.

Preferably, in formula (I)-1-A, formula (I)-1-B and formula (I)-1-C, only one moiety containing L′ is comprised, and the L′ group is attached to R^{H}.

In embodiments, the present invention provides a cationic lipid of formula (I)-1, (I)-1-A, (I)-1-B, (I)-1-C, (I), (IA), (IB), (IC), (II), (III), (IV) or (V), or a salt, hydrate, solvate, polymorph, optical isomer, geometrical isomer, enantiomer, diastereomer, tautomer, isotope labeled compound or mixtures thereof, wherein R^{T1} and R^{T2} are each independently selected from the group consisting of:

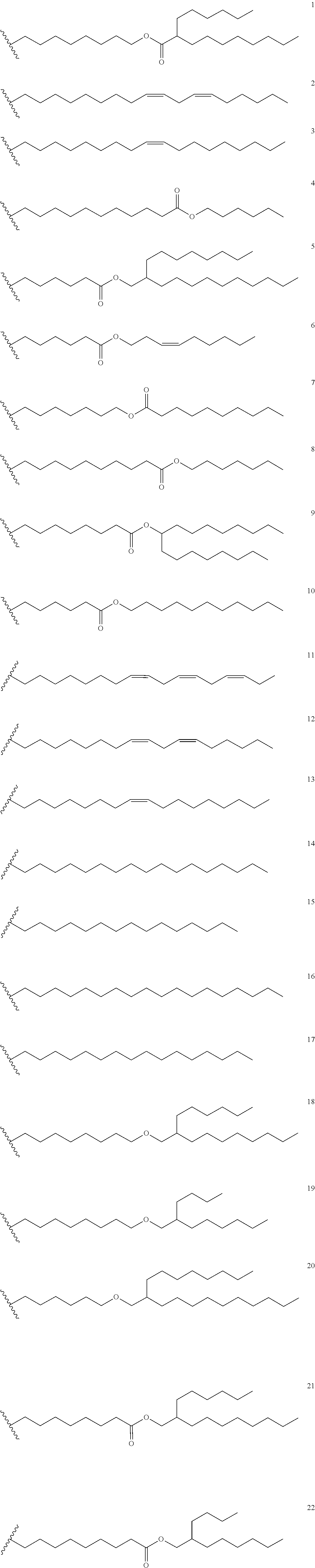

In embodiments, the present invention provides a cationic lipid of formula (I)-1, (I)-1-A, (I)-1-B, (I)-1-C, (I), (IA), (IB), (IC), (II), (III), (IV), or (V) or a salt, hydrate, solvate, polymorph, optical isomer, geometrical isomer, enantiomer, diastereomer, tautomer, isotope labeled compound or mixtures thereof, wherein R^{H} is —N(CH₃)₂, —N(C₂H₅)₂,

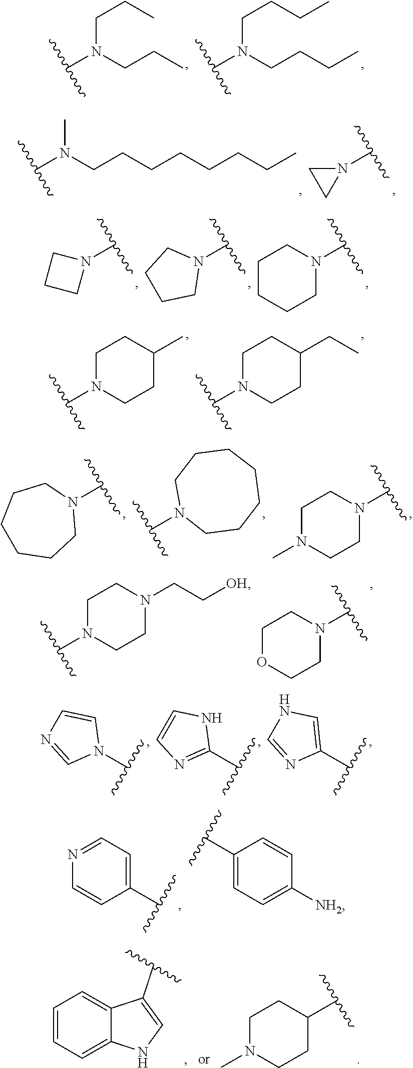

In some aspects, the present invention provides a cationic lipid, or a salt, hydrate, solvate, polymorph, optical isomer, geometrical isomer, enantiomer, diastereomer, tautomer, isotope labeled compound or mixtures thereof, wherein said cationic lipid is a compound selected from below Table 1A:

| No. | Structure |
|---|---|
| 457. | 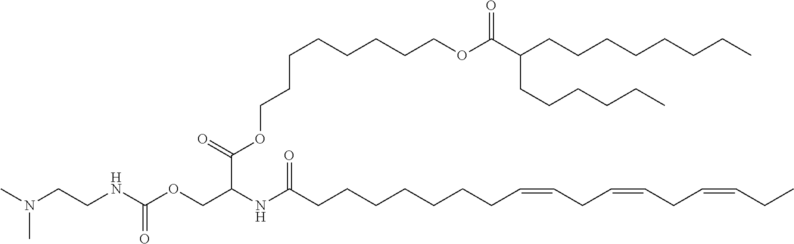 [SMILES: CC/C=C\C/C=C\C/C=C\CCCCCCCC(=O)NC(COC(=O)NCCN(C)C)C(=O)OCCCCCCCCOC(=O)C(CCCCCC)CCCCCCCC] |
| 494. | 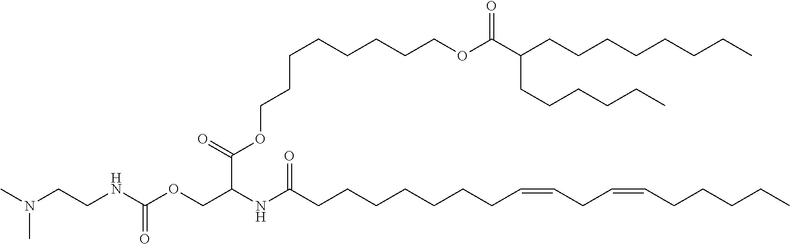 [SMILES: CCCCC/C=C\C/C=C\CCCCCCCC(=O)NC(COC(=O)NCCN(C)C)C(=O)OCCCCCCCCOC(=O)C(CCCCCC)CCCCCCCC] |
| 495. | 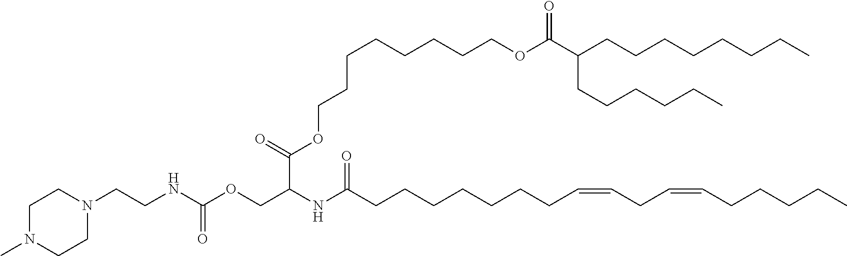 [SMILES: CCCCC/C=C\C/C=C\CCCCCCCC(=O)NC(COC(=O)NCCN1CCN(C)CC1)C(=O)OCCCCCCCCOC(=O)C(CCCCCC)CCCCCCCC] |
| 496. | 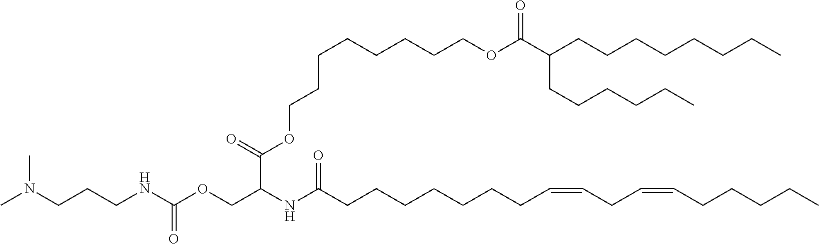 [SMILES: CCCCC/C=C\C/C=C\CCCCCCCC(=O)NC(COC(=O)NCCCN(C)C)C(=O)OCCCCCCCCOC(=O)C(CCCCCC)CCCCCCCC] |
| 497. | 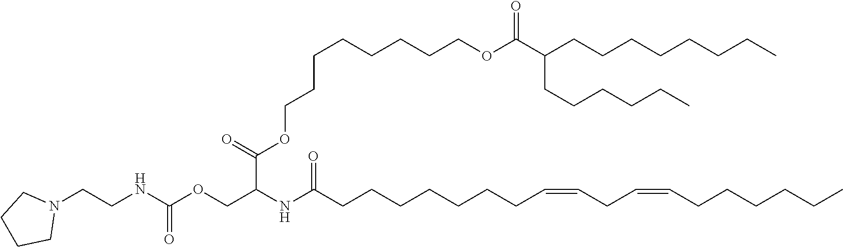 [SMILES: CCCCCC/C=C\C/C=C\CCCCCCCC(=O)NC(COC(=O)NCCN1CCCC1)C(=O)OCCCCCCCCOC(=O)C(CCCCCC)CCCCCCCC] |
| 498. | 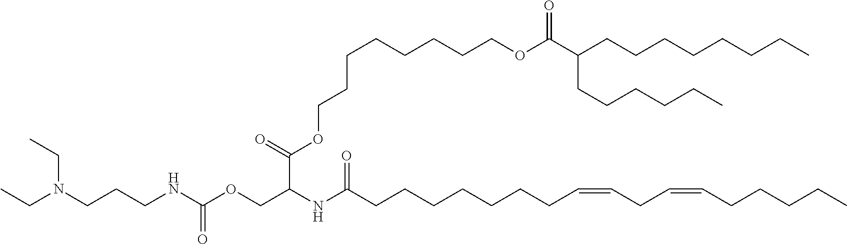 [SMILES: CCCCC/C=C\C/C=C\CCCCCCCC(=O)NC(COC(=O)NCCCN(CC)CC)C(=O)OCCCCCCCCOC(=O)C(CCCCCC)CCCCCCCC] |
| 499. | 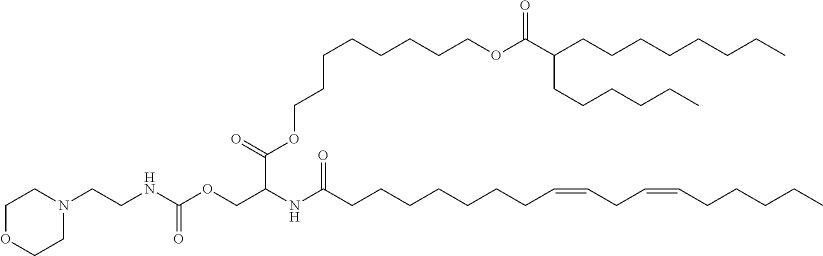 [SMILES: CCCCC/C=C\C/C=C\CCCCCCCC(=O)NC(COC(=O)NCCN1CCOCC1)C(=O)OCCCCCCCCOC(=O)C(CCCCCC)CCCCCCCC] |
| 500. | 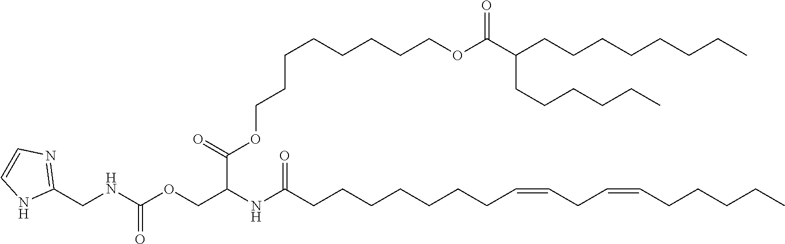 [SMILES: CCCCC/C=C\C/C=C\CCCCCCCC(=O)NC(COC(=O)NCc1ncc[nH]1)C(=O)OCCCCCCCCOC(=O)C(CCCCCC)CCCCCCCC] |
| 501. | 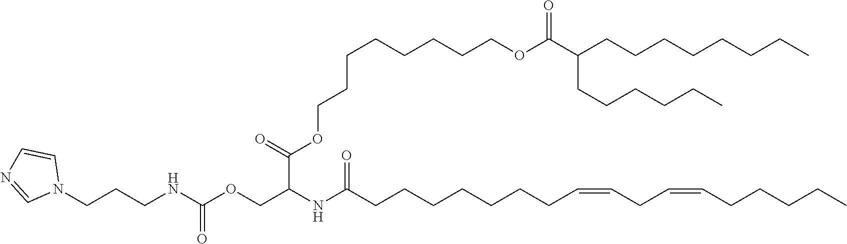 [SMILES: CCCCC/C=C\C/C=C\CCCCCCCC(=O)NC(COC(=O)NCCCn1ccnc1)C(=O)OCCCCCCCCOC(=O)C(CCCCCC)CCCCCCCC] |
| 547. | 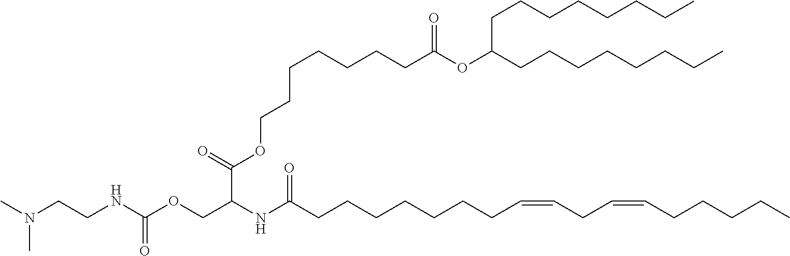 [SMILES: CCCCC/C=C\C/C=C\CCCCCCCC(=O)NC(COC(=O)NCCN(C)C)C(=O)OCCCCCCCC(=O)OC(CCCCCCCC)CCCCCCCC] |
| 548. | 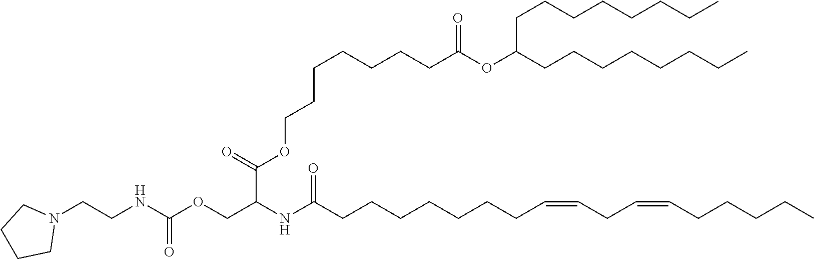 [SMILES: CCCCC/C=C\C/C=C\CCCCCCCC(=O)NC(COC(=O)NCCN1CCCC1)C(=O)OCCCCCCCC(=O)OC(CCCCCCCC)CCCCCCCC] |
| 549. | 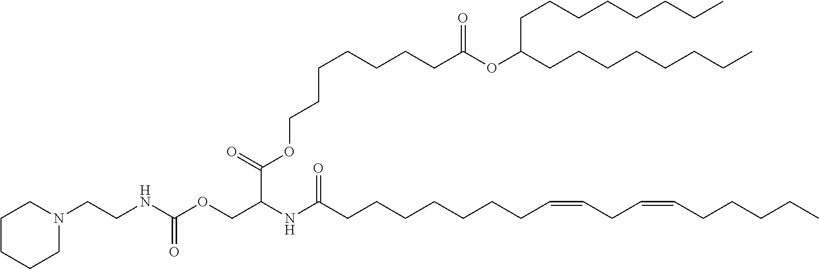 [SMILES: CCCCC/C=C\C/C=C\CCCCCCCC(=O)NC(COC(=O)NCCN1CCCCC1)C(=O)OCCCCCCCC(=O)OC(CCCCCCCC)CCCCCCCC] |
| 550. | 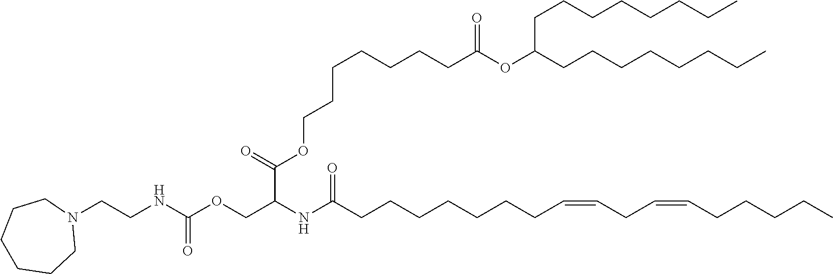 [SMILES: CCCCC/C=C\C/C=C\CCCCCCCC(=O)NC(COC(=O)NCCN1CCCCCC1)C(=O)OCCCCCCCC(=O)OC(CCCCCCCC)CCCCCCCC] |
| 551. | 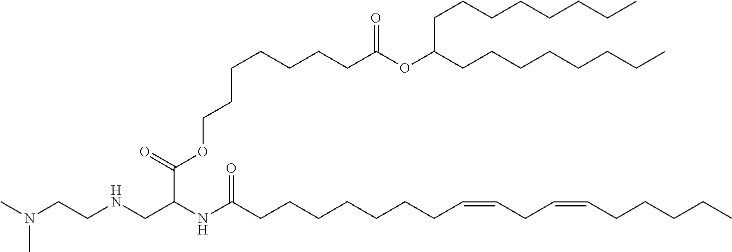 [SMILES: CCCCC/C=C\C/C=C\CCCCCCCC(=O)NC(CNCCN(C)C)C(=O)OCCCCCCCC(=O)OC(CCCCCCCC)CCCCCCCC] |
| 556. | 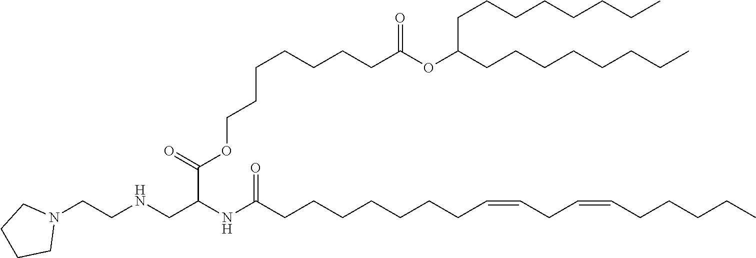 [SMILES: CCCCC/C=C\C/C=C\CCCCCCCC(=O)NC(CNCCN1CCCC1)C(=O)OCCCCCCCC(=O)OC(CCCCCCCC)CCCCCCCC] |
| 557. | 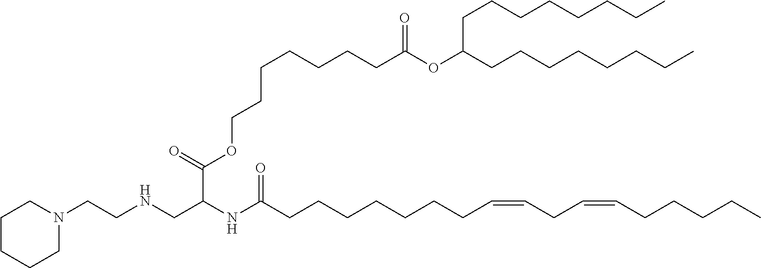 [SMILES: CCCCC/C=C\C/C=C\CCCCCCCC(=O)NC(CNCCN1CCCCC1)C(=O)OCCCCCCCC(=O)OC(CCCCCCCC)CCCCCCCC] |
| 558. | 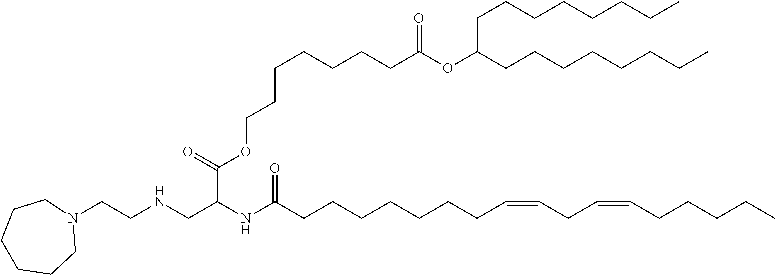 [SMILES: CCCCC/C=C\C/C=C\CCCCCCCC(=O)NC(CNCCN1CCCCCC1)C(=O)OCCCCCCCC(=O)OC(CCCCCCCC)CCCCCCCC] |
| 563. | 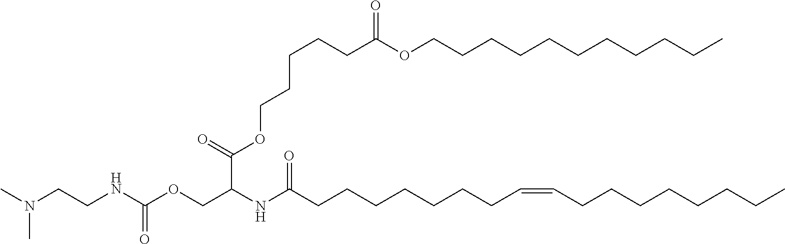 [SMILES: CCCCCCCC/C=C\CCCCCCCC(=O)NC(COC(=O)NCCN(C)C)C(=O)OCCCCCC(=O)OCCCCCCCCCCC] |
| 564. | 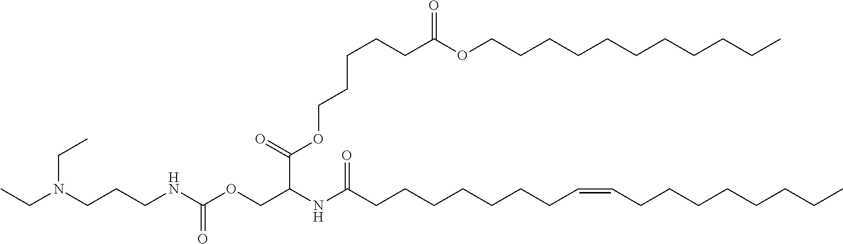 [SMILES: CCCCCCCC/C=C\CCCCCCCC(=O)NC(COC(=O)NCCCN(CC)CC)C(=O)OCCCCCC(=O)OCCCCCCCCCCC] |
| 565. | 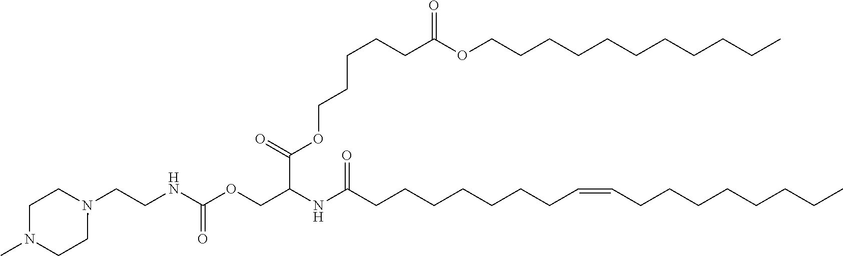 [SMILES: CCCCCCCC/C=C\CCCCCCCC(=O)NC(COC(=O)NCCN1CCN(C)CC1)C(=O)OCCCCCC(=O)OCCCCCCCCCCC] |
| 566. | 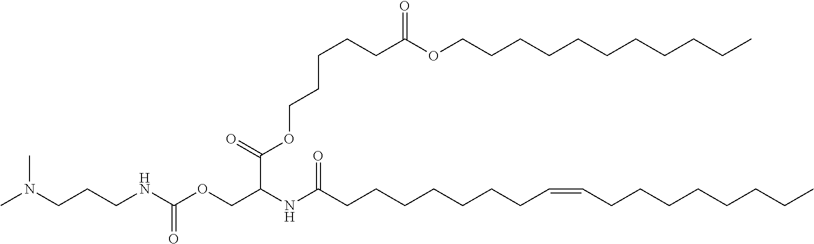 [SMILES: CCCCCCCC/C=C\CCCCCCCC(=O)NC(COC(=O)NCCCN(C)C)C(=O)OCCCCCC(=O)OCCCCCCCCCCC] |
| 567. | 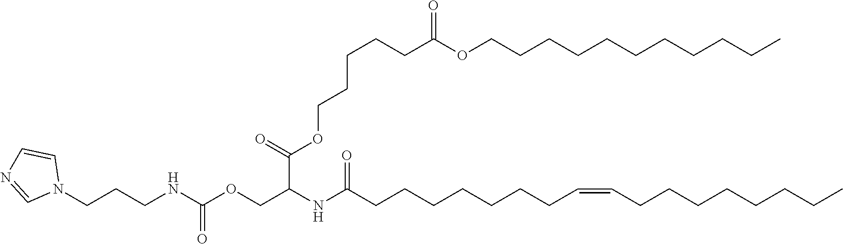 [SMILES: CCCCCCCC/C=C\CCCCCCCC(=O)NC(COC(=O)NCCCn1ccnc1)C(=O)OCCCCCC(=O)OCCCCCCCCCCC] |
| 568. | 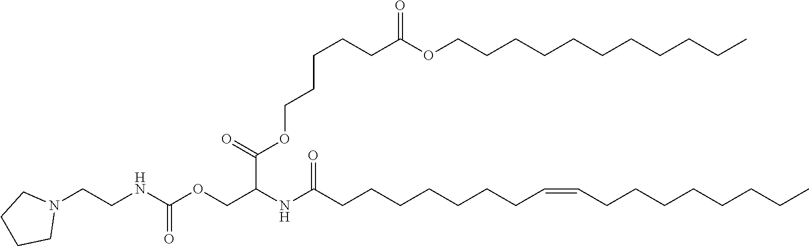 [SMILES: CCCCCCCC/C=C\CCCCCCCC(=O)NC(COC(=O)NCCN1CCCC1)C(=O)OCCCCCC(=O)OCCCCCCCCCCC] |
| 569. | 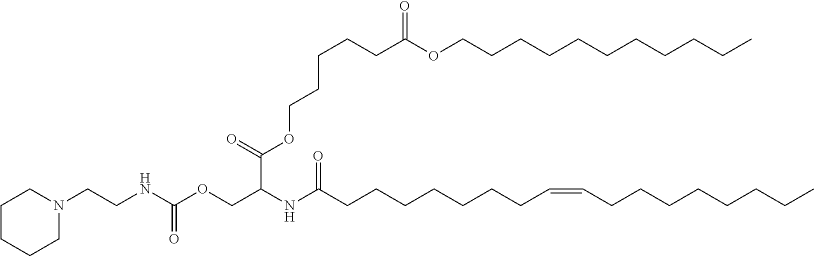 [SMILES: CCCCCCCC/C=C\CCCCCCCC(=O)NC(COC(=O)NCCN1CCCCC1)C(=O)OCCCCCC(=O)OCCCCCCCCCCC] |
| 594. | 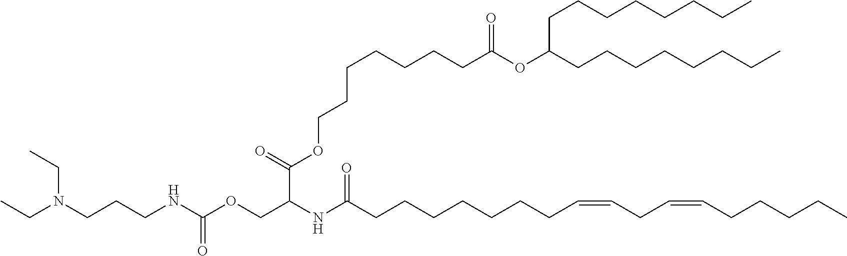 [SMILES: CCCCC/C=C\C/C=C\CCCCCCCC(=O)NC(COC(=O)NCCCN(CC)CC)C(=O)OCCCCCCCC(=O)OC(CCCCCCCC)CCCCCCCC] |
| 595. | 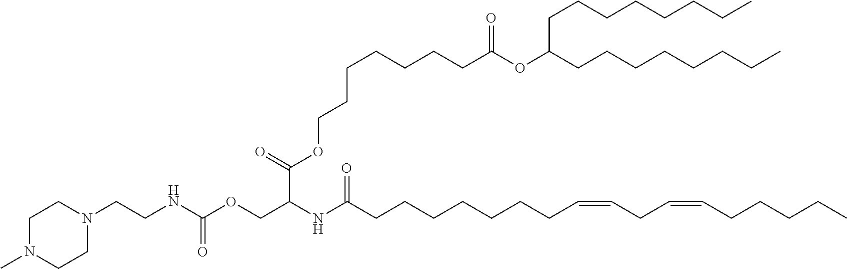 [SMILES: CCCCC/C=C\C/C=C\CCCCCCCC(=O)NC(COC(=O)NCCN1CCN(C)CC1)C(=O)OCCCCCCCC(=O)OC(CCCCCCCC)CCCCCCCC] |
| 596. | 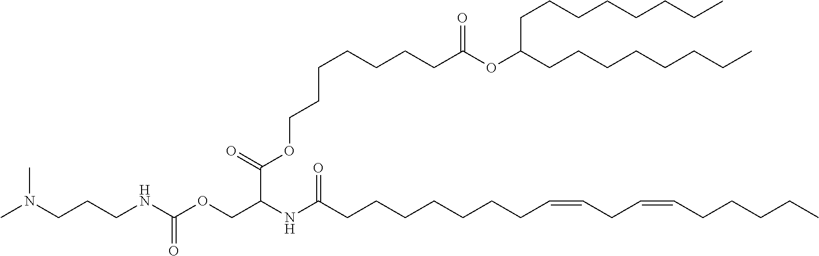 [SMILES: CCCCC/C=C\C/C=C\CCCCCCCC(=O)NC(COC(=O)NCCCN(C)C)C(=O)OCCCCCCCC(=O)OC(CCCCCCCC)CCCCCCCC] |
| 601. | 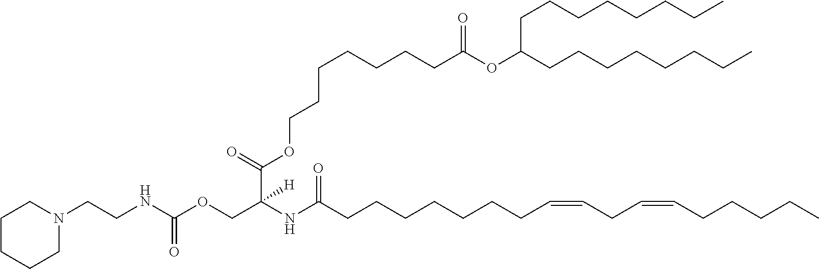 [SMILES: CCCCC/C=C\C/C=C\CCCCCCCC(=O)N[C@@H](COC(=O)NCCN1CCCCC1)C(=O)OCCCCCCCC(=O)OC(CCCCCCCC)CCCCCCCC] |
| 602. | 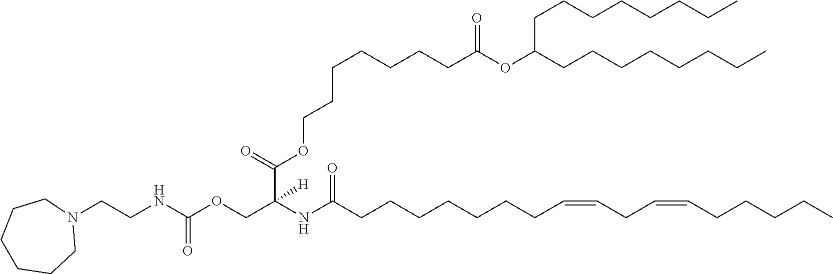 [SMILES: CCCCC/C=C\C/C=C\CCCCCCCC(=O)N[C@@H](COC(=O)NCCN1CCCCCC1)C(=O)OCCCCCCCC(=O)OC(CCCCCCCC)CCCCCCCC] |
| 611. | 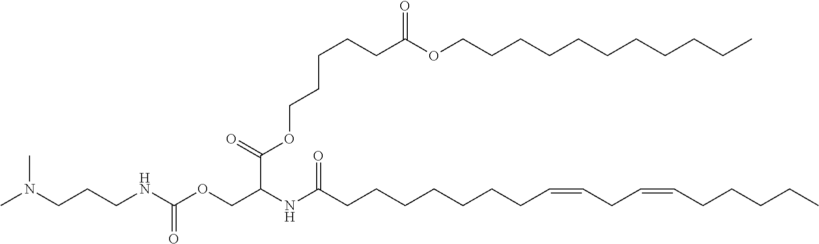 [SMILES: CCCCC/C=C\C/C=C\CCCCCCCC(=O)NC(COC(=O)NCCCN(C)C)C(=O)OCCCCCC(=O)OCCCCCCCCCCC] |
| 612. | 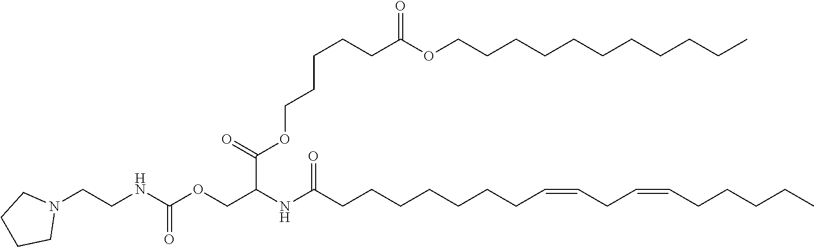 [SMILES: CCCCC/C=C\C/C=C\CCCCCCCC(=O)NC(COC(=O)NCCN1CCCC1)C(=O)OCCCCCC(=O)OCCCCCCCCCCC] |
| 613. | 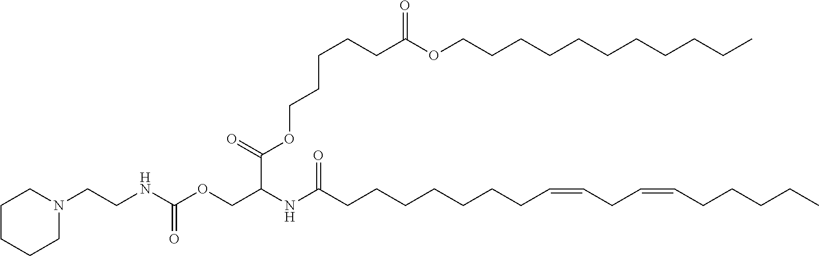 [SMILES: CCCCC/C=C\C/C=C\CCCCCCCC(=O)NC(COC(=O)NCCN1CCCCC1)C(=O)OCCCCCC(=O)OCCCCCCCCCCC] |
| 614. | 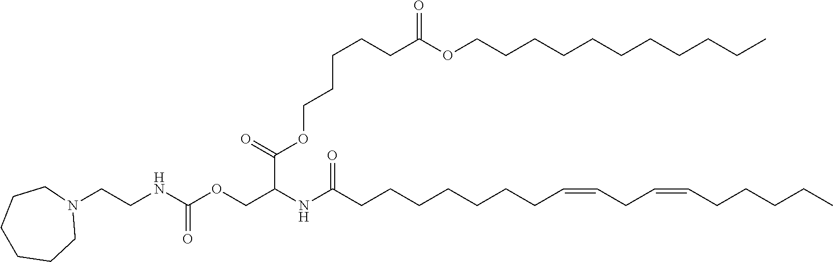 [SMILES: CCCCC/C=C\C/C=C\CCCCCCCC(=O)NC(COC(=O)NCCN1CCCCCC1)C(=O)OCCCCCC(=O)OCCCCCCCCCCC] |
| 619. | 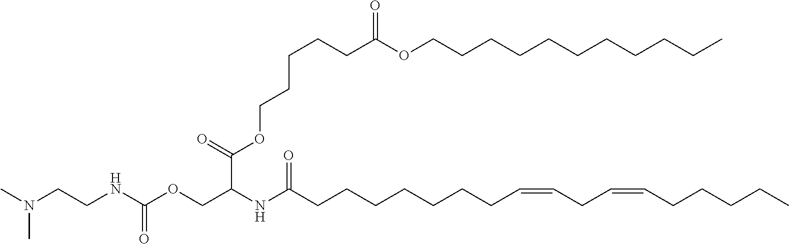 [SMILES: CCCCC/C=C\C/C=C\CCCCCCCC(=O)NC(COC(=O)NCCN(C)C)C(=O)OCCCCCC(=O)OCCCCCCCCCCC] |
| 620. | 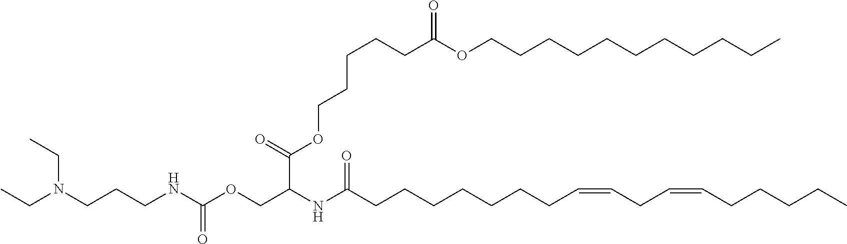 [SMILES: CCCCC/C=C\C/C=C\CCCCCCCC(=O)NC(COC(=O)NCCCN(CC)CC)C(=O)OCCCCCC(=O)OCCCCCCCCCCC] |
| 621. | 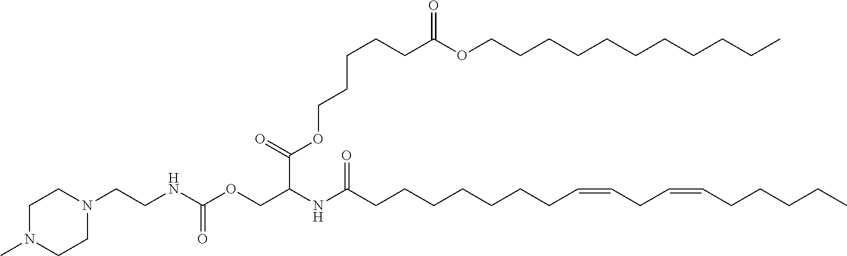 [SMILES: CCCCC/C=C\C/C=C\CCCCCCCC(=O)NC(COC(=O)NCCN1CCN(C)CC1)C(=O)OCCCCCC(=O)OCCCCCCCCCCC] |
| 622. | 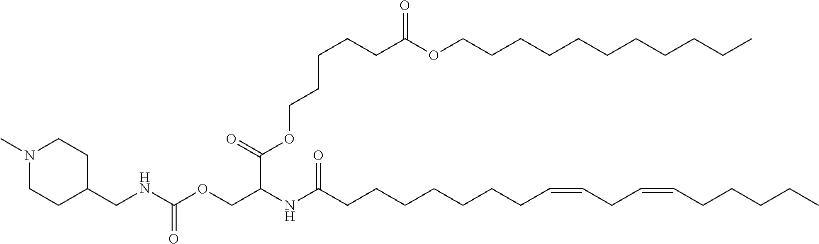 [SMILES: CCCCC/C=C\C/C=C\CCCCCCCC(=O)NC(COC(=O)NCC1CCN(C)CC1)C(=O)OCCCCCC(=O)OCCCCCCCCCCC] |
| 695. | 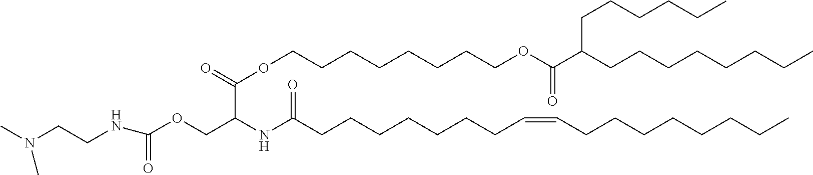 |
| 696. | 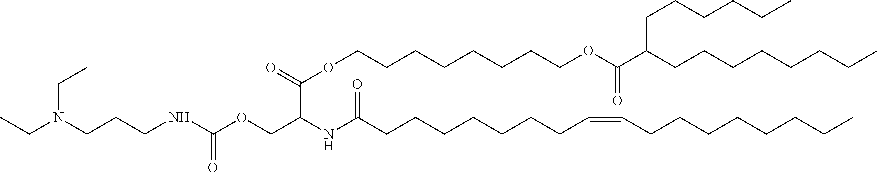 |
| 697. | 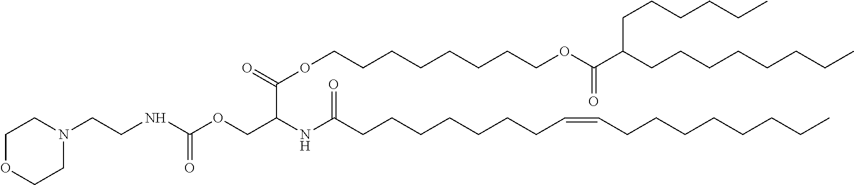 |
| 698. | 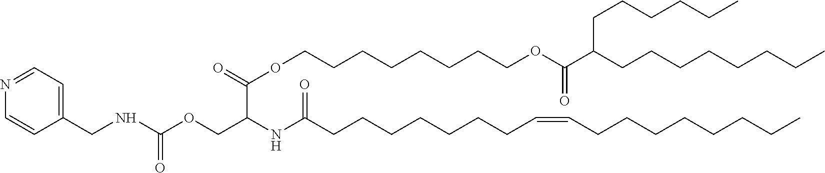 |
| 699. | 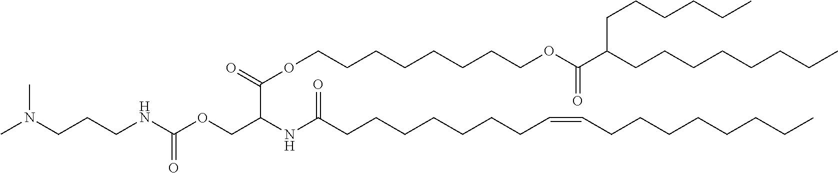 |
| 700. | 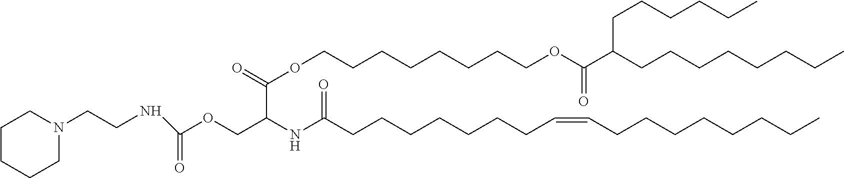 |
| 701. | 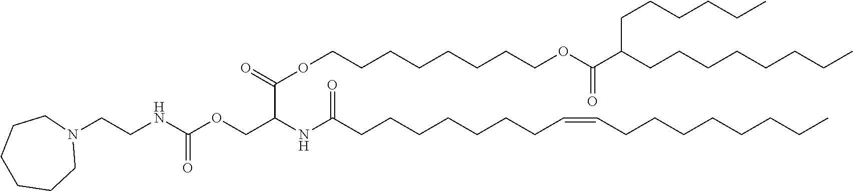 |
| 702. | 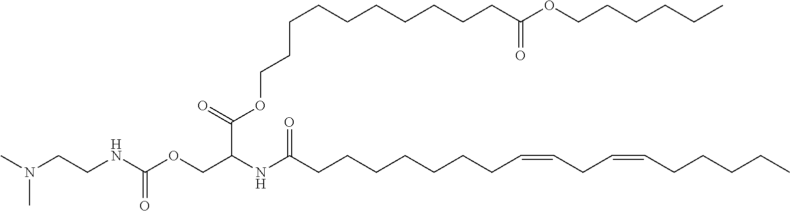 |
| 703. | 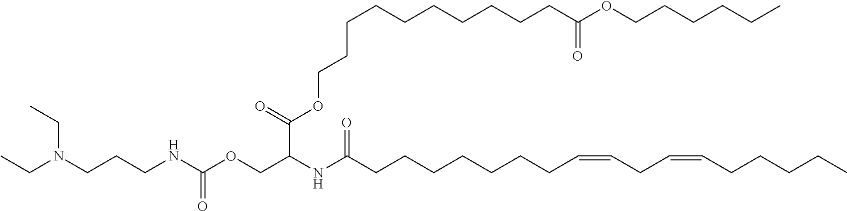 [SMILES: CCCCC/C=C\C/C=C\CCCCCCCC(=O)NC(COC(=O)NCCCN(CC)CC)C(=O)OCCCCCCCCCCC(=O)OCCCCCC] |
| 704. | 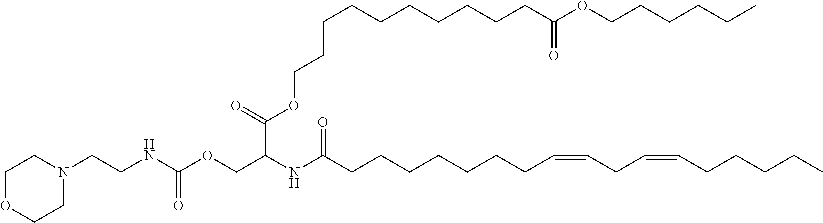 [SMILES: CCCCC/C=C\C/C=C\CCCCCCCC(=O)NC(COC(=O)NCCN1CCOCC1)C(=O)OCCCCCCCCCCC(=O)OCCCCCC] |
| 705. | 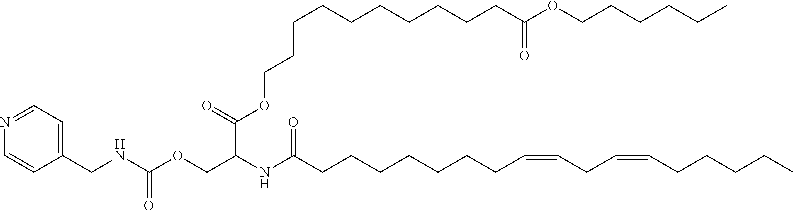 [SMILES: CCCCC/C=C\C/C=C\CCCCCCCC(=O)NC(COC(=O)NCc1ccncc1)C(=O)OCCCCCCCCCCC(=O)OCCCCCC] |
| 706. | 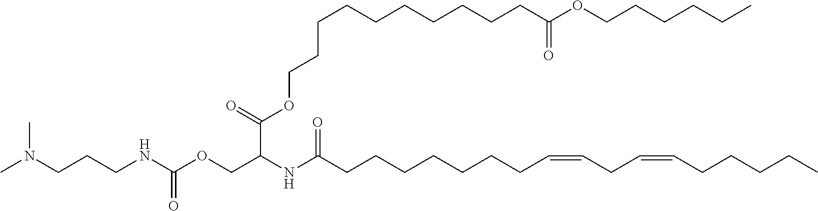 [SMILES: CCCCC/C=C\C/C=C\CCCCCCCC(=O)NC(COC(=O)NCCCN(C)C)C(=O)OCCCCCCCCCCC(=O)OCCCCCC] |
| 707. | 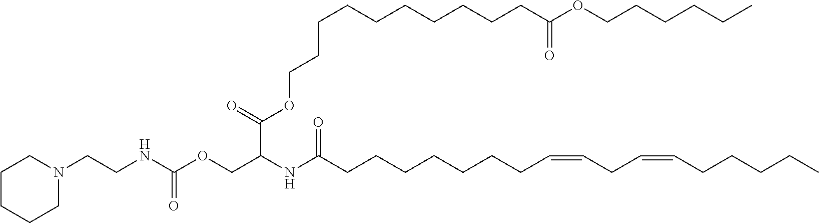 [SMILES: CCCCC/C=C\C/C=C\CCCCCCCC(=O)NC(COC(=O)NCCN1CCCCC1)C(=O)OCCCCCCCCCCC(=O)OCCCCCC] |
| 708. | 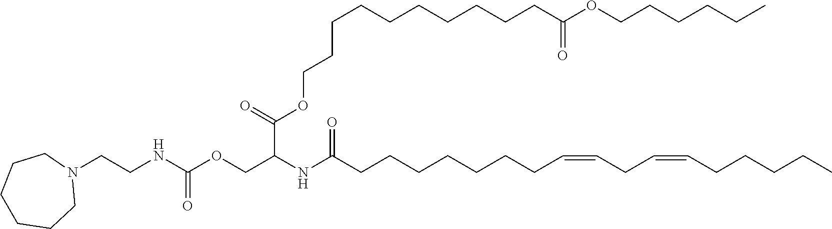 [SMILES: CCCCC/C=C\C/C=C\CCCCCCCC(=O)NC(COC(=O)NCCN1CCCCCC1)C(=O)OCCCCCCCCCCC(=O)OCCCCCC] |
| 709. | 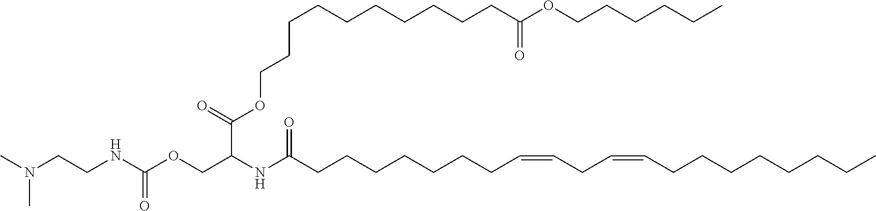 [SMILES: CCCCCCCC/C=C\C/C=C\CCCCCCCC(=O)NC(COC(=O)NCCN(C)C)C(=O)OCCCCCCCCCCC(=O)OCCCCCC] |
| 710. | 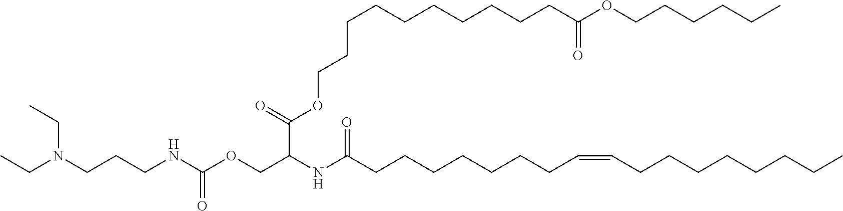 [SMILES: CCCCCCCC/C=C\CCCCCCCC(=O)NC(COC(=O)NCCCN(CC)CC)C(=O)OCCCCCCCCCCC(=O)OCCCCCC] |
| 711. | 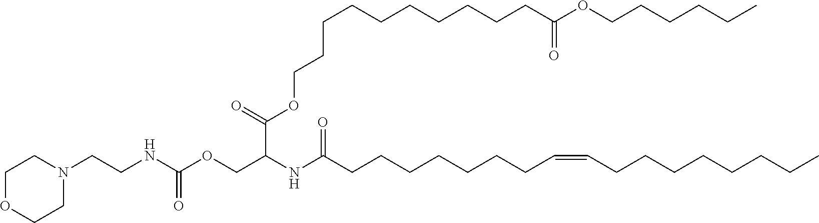 [SMILES: CCCCCCCC/C=C\CCCCCCCC(=O)NC(COC(=O)NCCN1CCOCC1)C(=O)OCCCCCCCCCCC(=O)OCCCCCC] |
| 712. | 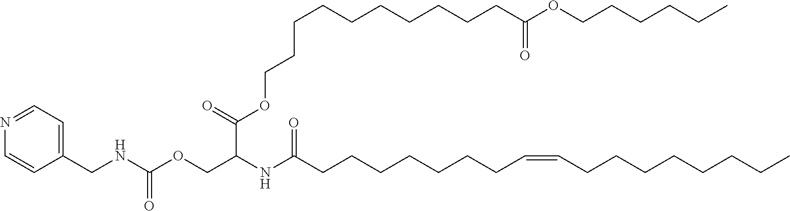 [SMILES: CCCCCCCC/C=C\CCCCCCCC(=O)NC(COC(=O)NCc1ccncc1)C(=O)OCCCCCCCCCCC(=O)OCCCCCC] |
| 713. | 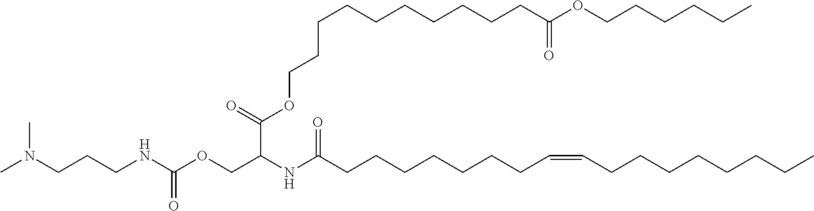 [SMILES: CCCCCCCC/C=C\CCCCCCCC(=O)NC(COC(=O)NCCCN(C)C)C(=O)OCCCCCCCCCCC(=O)OCCCCCC] |
| 714. | 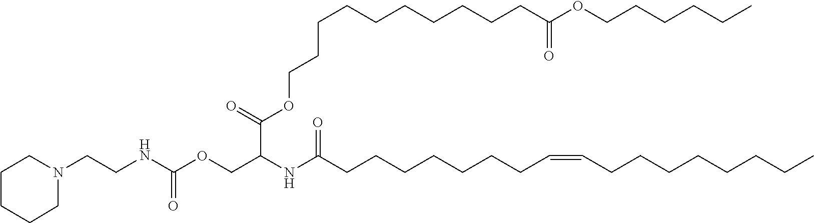 [SMILES: CCCCCCCC/C=C\CCCCCCCC(=O)NC(COC(=O)NCCN1CCCCC1)C(=O)OCCCCCCCCCCC(=O)OCCCCCC] |
| 715. | 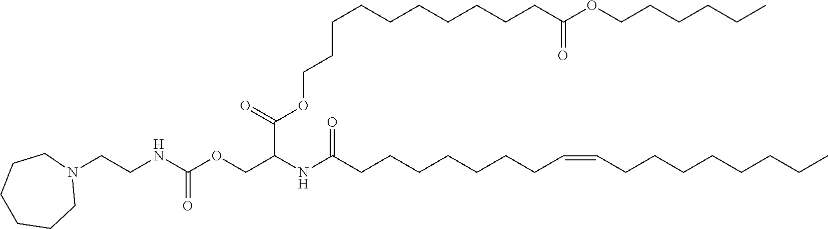 [SMILES: CCCCCCCC/C=C\CCCCCCCC(=O)NC(COC(=O)NCCN1CCCCCC1)C(=O)OCCCCCCCCCCC(=O)OCCCCCC] |
| 716. | 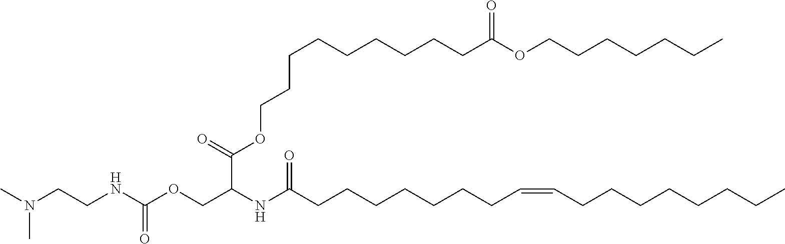 [SMILES: CCCCCCCC/C=C\CCCCCCCC(=O)NC(COC(=O)NCCN(C)C)C(=O)OCCCCCCCCCC(=O)OCCCCCCC] |
| 717. | 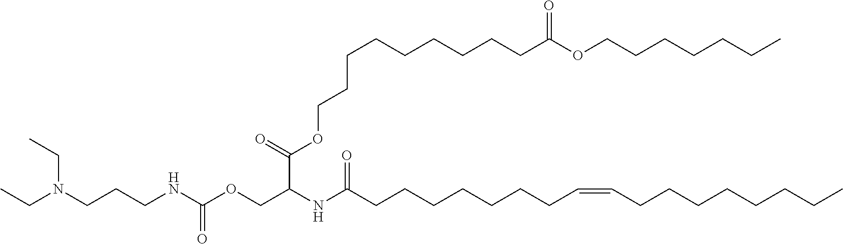 [SMILES: CCCCCCCC/C=C\CCCCCCCC(=O)NC(COC(=O)NCCCN(CC)CC)C(=O)OCCCCCCCCCC(=O)OCCCCCCC] |
| 718. | 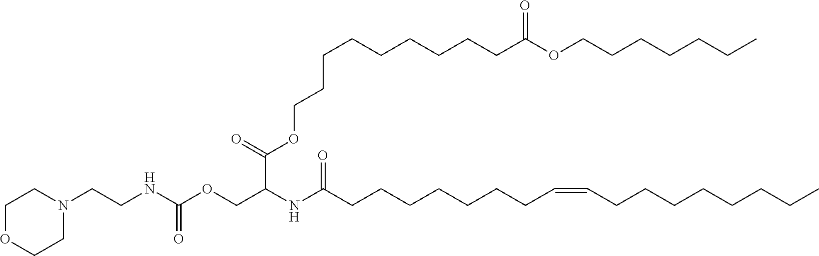 [SMILES: CCCCCCCC/C=C\CCCCCCCC(=O)NC(COC(=O)NCCN1CCOCC1)C(=O)OCCCCCCCCCC(=O)OCCCCCCC] |
| 719. | 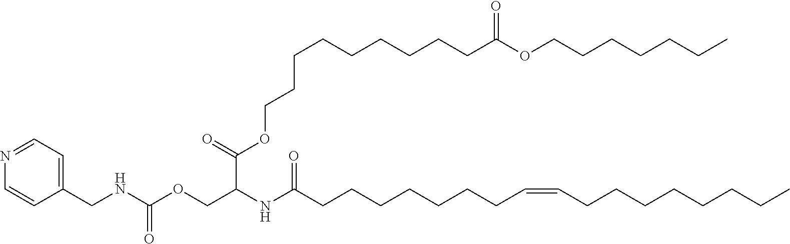 [SMILES: CCCCCCCC/C=C\CCCCCCCC(=O)NC(COC(=O)NCc1ccncc1)C(=O)OCCCCCCCCCC(=O)OCCCCCCC] |
| 720. | 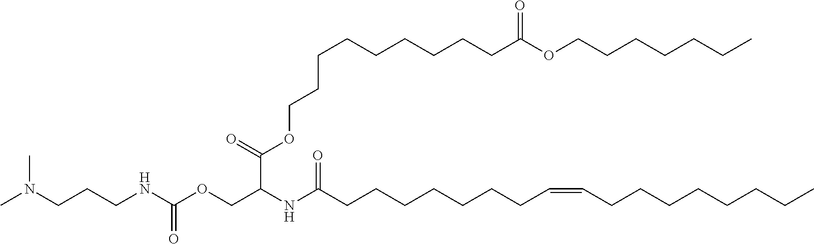 [SMILES: CCCCCCCC/C=C\CCCCCCCC(=O)NC(COC(=O)NCCCN(C)C)C(=O)OCCCCCCCCCC(=O)OCCCCCCC] |
| 721. | 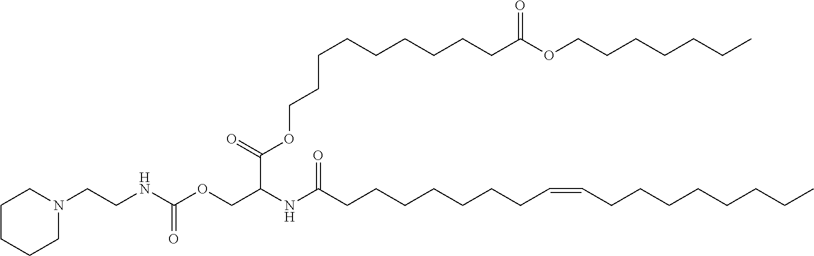 [SMILES: CCCCCCCC/C=C\CCCCCCCC(=O)NC(COC(=O)NCCN1CCCCC1)C(=O)OCCCCCCCCCC(=O)OCCCCCCC] |
| 722. | 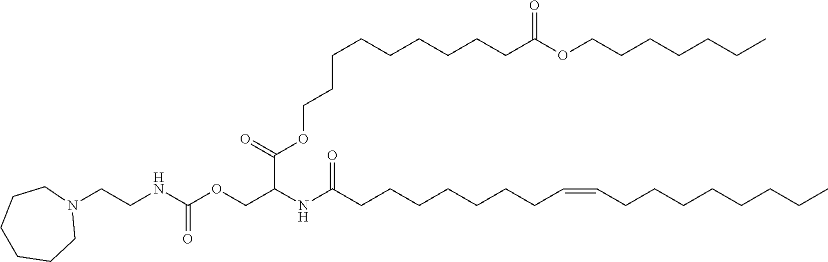 [SMILES: CCCCCCCC/C=C\CCCCCCCC(=O)NC(COC(=O)NCCN1CCCCCC1)C(=O)OCCCCCCCCCC(=O)OCCCCCCC] |
| 723. | 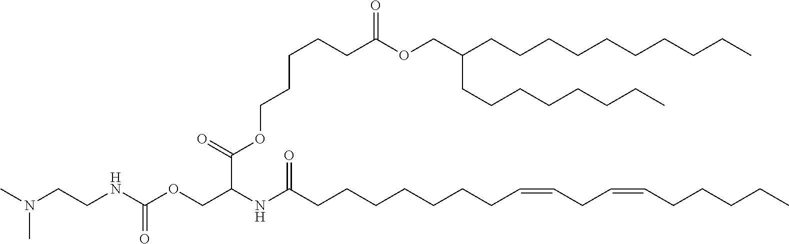 [SMILES: CCCCC/C=C\C/C=C\CCCCCCCC(=O)NC(COC(=O)NCCN(C)C)C(=O)OCCCCCC(=O)OCC(CCCCCCCC)CCCCCCCCCC] |
| 724. | 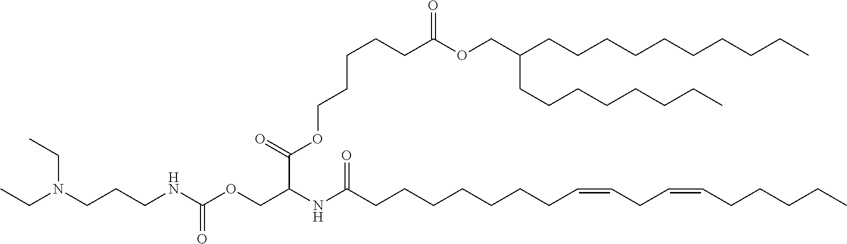 [SMILES: CCCCC/C=C\C/C=C\CCCCCCCC(=O)NC(COC(=O)NCCCN(CC)CC)C(=O)OCCCCCC(=O)OCC(CCCCCCCC)CCCCCCCCCC] |
| 725. | 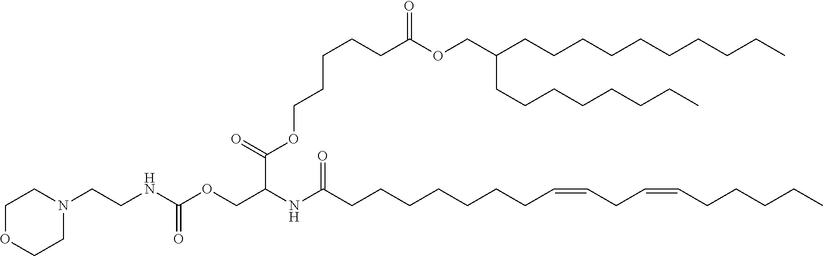 [SMILES: CCCCC/C=C\C/C=C\CCCCCCCC(=O)NC(COC(=O)NCCN1CCOCC1)C(=O)OCCCCCC(=O)OCC(CCCCCCCC)CCCCCCCCCC] |
| 726. | 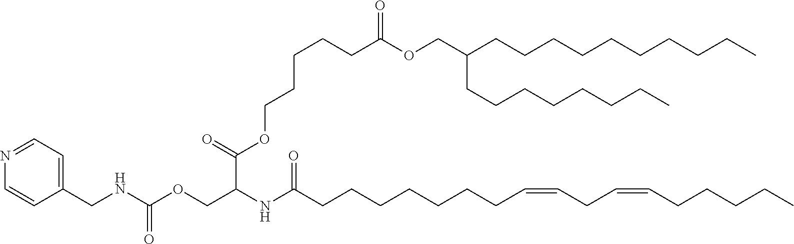 [SMILES: CCCCC/C=C\C/C=C\CCCCCCCC(=O)NC(COC(=O)NCc1ccncc1)C(=O)OCCCCCC(=O)OCC(CCCCCCCC)CCCCCCCCCC] |
| 727. | 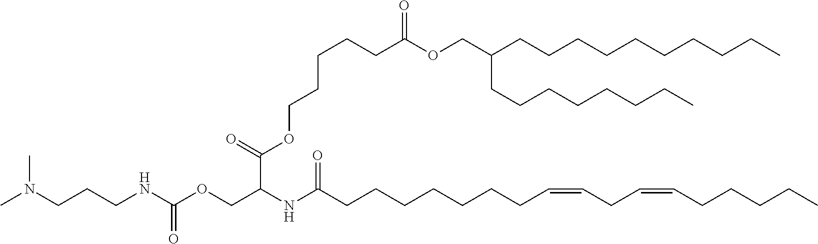 [SMILES: CCCCC/C=C\C/C=C\CCCCCCCC(=O)NC(COC(=O)NCCCN(C)C)C(=O)OCCCCCC(=O)OCC(CCCCCCCC)CCCCCCCCCC] |
| 728. | 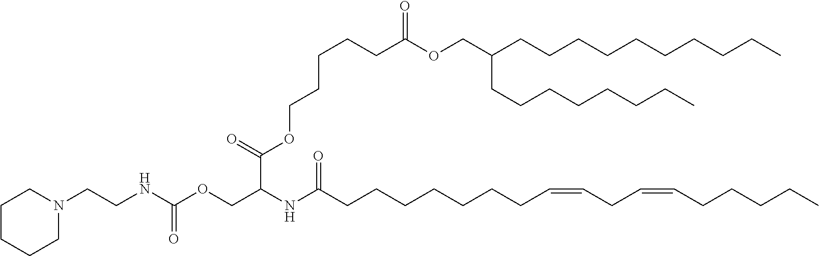 [SMILES: CCCCC/C=C\C/C=C\CCCCCCCC(=O)NC(COC(=O)NCCN1CCCCC1)C(=O)OCCCCCC(=O)OCC(CCCCCCCC)CCCCCCCCCC] |
| 729. | 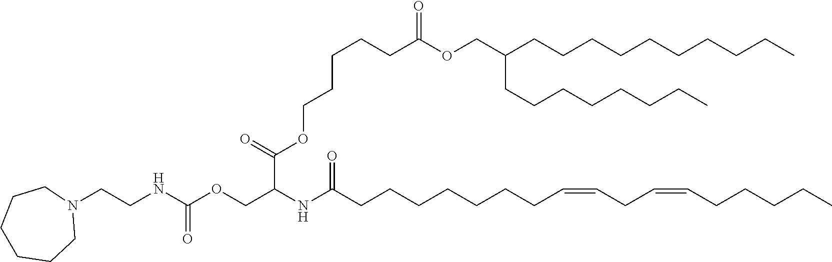 [SMILES: CCCCC/C=C\C/C=C\CCCCCCCC(=O)NC(COC(=O)NCCN1CCCCCC1)C(=O)OCCCCCC(=O)OCC(CCCCCCCC)CCCCCCCCCC] |
| 730. | 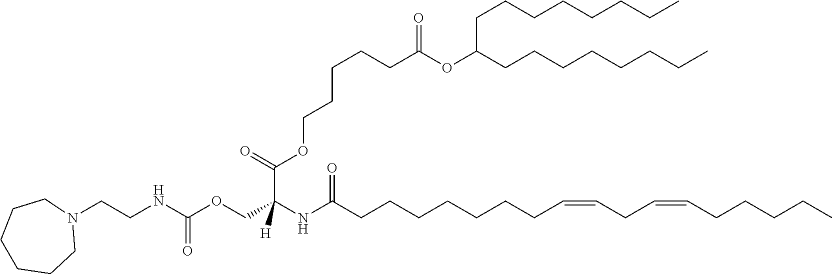 [SMILES: CCCCC/C=C\C/C=C\CCCCCCCC(=O)N[C@H](COC(=O)NCCN1CCCCCC1)C(=O)OCCCCCC(=O)OC(CCCCCCCC)CCCCCCCC] |
| 731. | 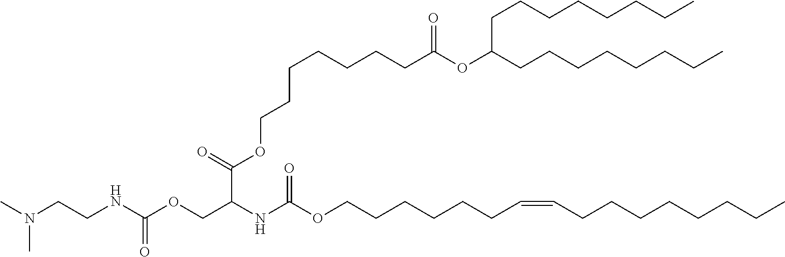 [SMILES: CCCCCCCC/C=C\CCCCCCOC(=O)NC(COC(=O)NCCN(C)C)C(=O)OCCCCCCCC(=O)OC(CCCCCCCC)CCCCCCCC] |
| 732. | 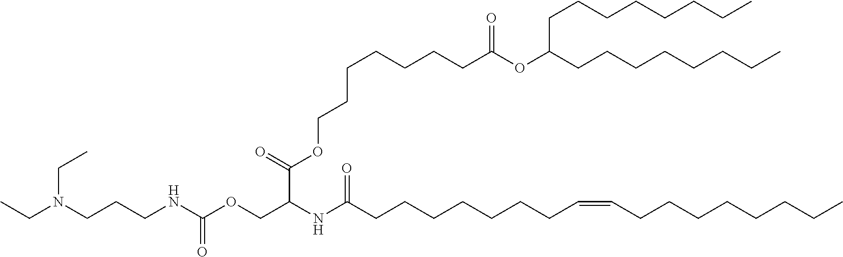 [SMILES: CCCCCCCC/C=C\CCCCCCCC(=O)NC(COC(=O)NCCCN(CC)CC)C(=O)OCCCCCCCC(=O)OC(CCCCCCCC)CCCCCCCC] |
| 733. | 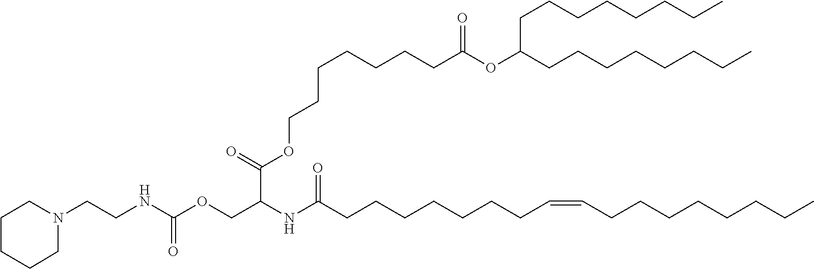 [SMILES: CCCCCCCC/C=C\CCCCCCCC(=O)NC(COC(=O)NCCN1CCCCC1)C(=O)OCCCCCCCC(=O)OC(CCCCCCCC)CCCCCCCC] |
| 734. | 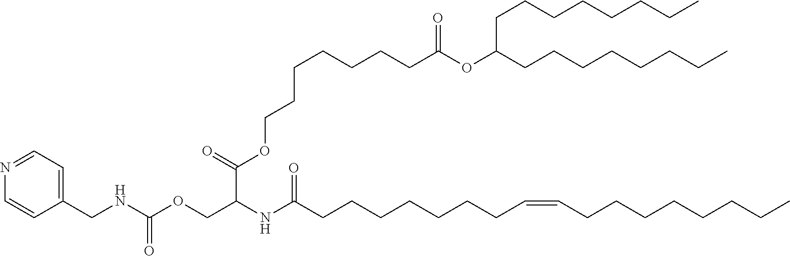 [SMILES: CCCCCCCC/C=C\CCCCCCCC(=O)NC(COC(=O)NCc1ccncc1)C(=O)OCCCCCCCC(=O)OC(CCCCCCCC)CCCCCCCC] |
| 735. | 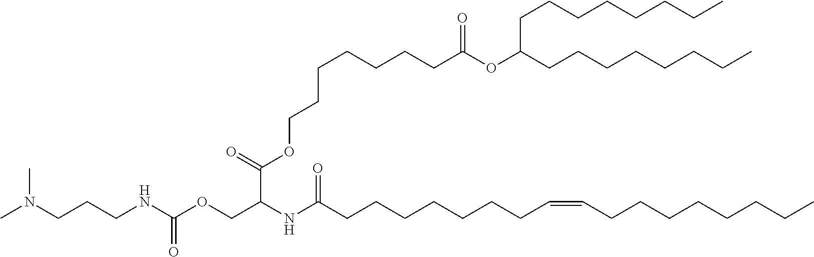 [SMILES: CCCCCCCC/C=C\CCCCCCCC(=O)NC(COC(=O)NCCCN(C)C)C(=O)OCCCCCCCC(=O)OC(CCCCCCCC)CCCCCCCC] |
| 736. | 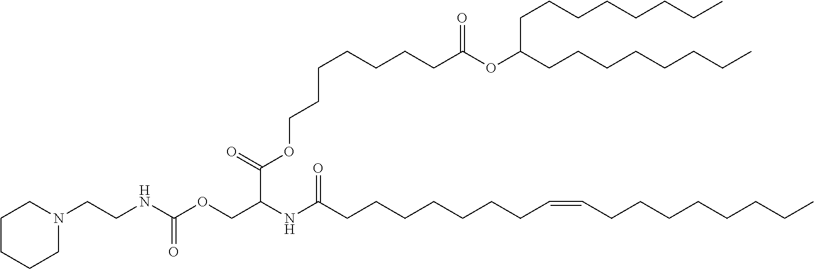 [SMILES: CCCCCCCC/C=C\CCCCCCCC(=O)NC(COC(=O)NCCN1CCCCC1)C(=O)OCCCCCCCC(=O)OC(CCCCCCCC)CCCCCCCC] |
| 737. | 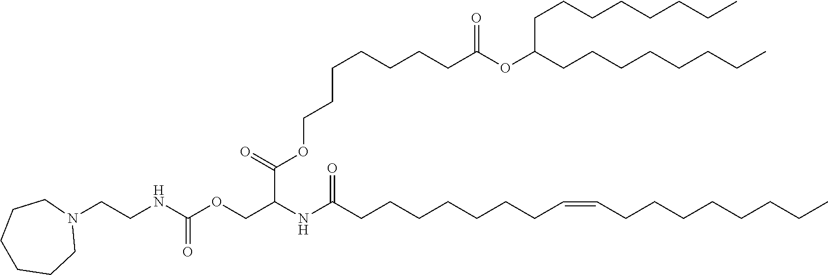 [SMILES: CCCCCCCC/C=C\CCCCCCCC(=O)NC(COC(=O)NCCN1CCCCCC1)C(=O)OCCCCCCCC(=O)OC(CCCCCCCC)CCCCCCCC] |
| 738. | 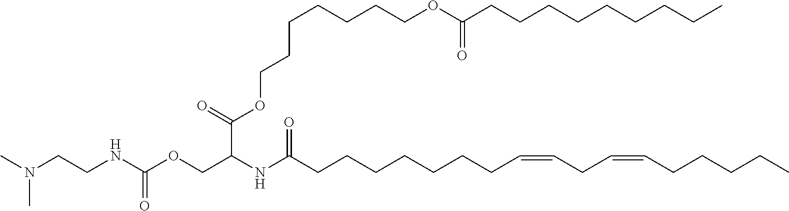 [SMILES: CCCCC/C=C\C/C=C\CCCCCCCC(=O)NC(COC(=O)NCCN(C)C)C(=O)OCCCCCCCOC(=O)CCCCCCCCC] |
| 739. | 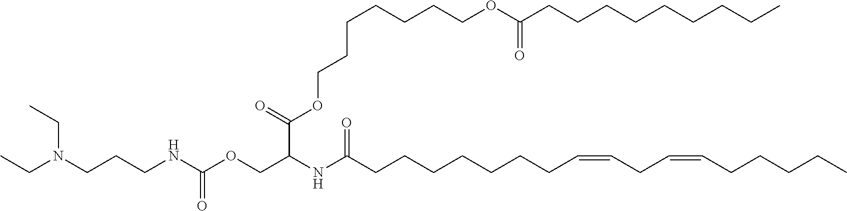 [SMILES: CCCCC/C=C\C/C=C\CCCCCCCC(=O)NC(COC(=O)NCCCN(CC)CC)C(=O)OCCCCCCCOC(=O)CCCCCCCCC] |
| 740. | 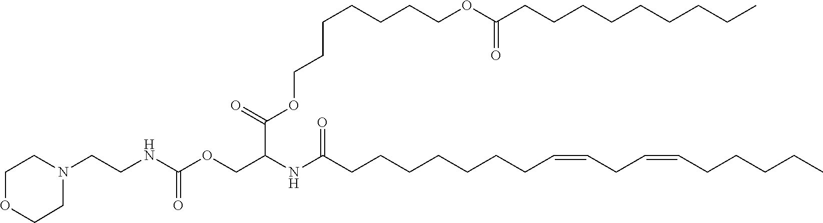 [SMILES: CCCCC/C=C\C/C=C\CCCCCCCC(=O)NC(COC(=O)NCCN1CCOCC1)C(=O)OCCCCCCCOC(=O)CCCCCCCCC] |
| 741. | 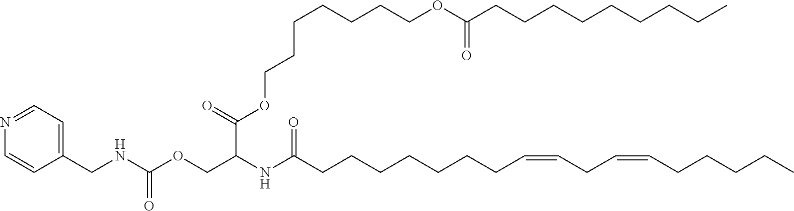 [SMILES: CCCCC/C=C\C/C=C\CCCCCCCC(=O)NC(COC(=O)NCc1ccncc1)C(=O)OCCCCCCCOC(=O)CCCCCCCCC] |
| 742. | 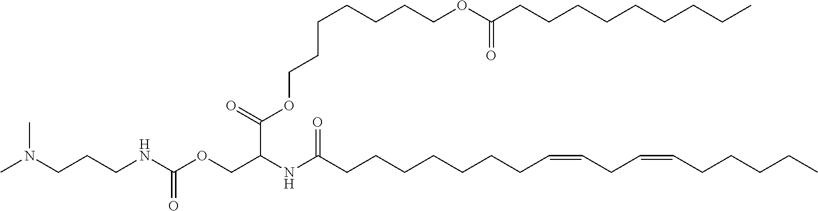 [SMILES: CCCCC/C=C\C/C=C\CCCCCCCC(=O)NC(COC(=O)NCCCN(C)C)C(=O)OCCCCCCCOC(=O)CCCCCCCCC] |
| 743. | 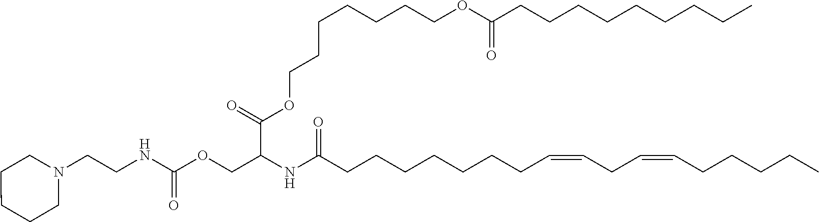 [SMILES: CCCCC/C=C\C/C=C\CCCCCCCC(=O)NC(COC(=O)NCCN1CCCCC1)C(=O)OCCCCCCCOC(=O)CCCCCCCCC] |
| 744. | 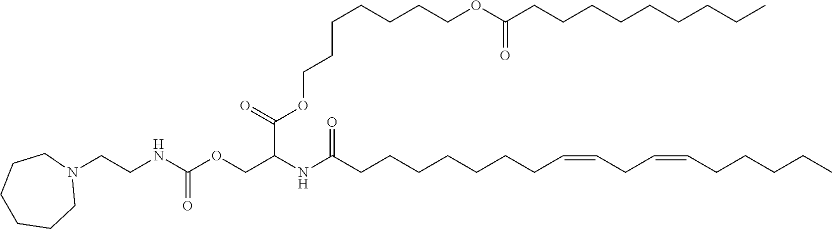 [SMILES: CCCCC/C=C\C/C=C\CCCCCCCC(=O)NC(COC(=O)NCCN1CCCCCC1)C(=O)OCCCCCCCOC(=O)CCCCCCCCC] |
| 745. | 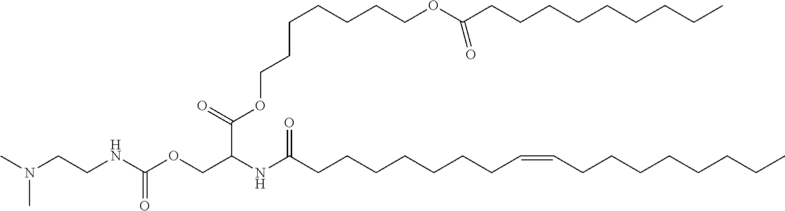 |
| 746. | 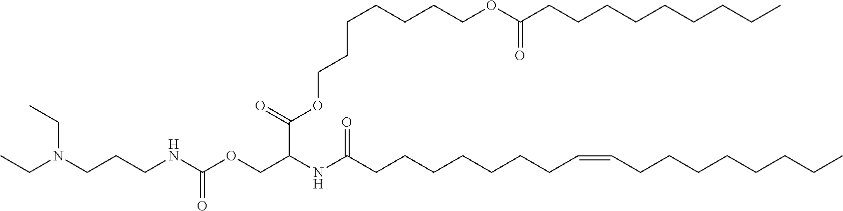 [SMILES: CCCCCCCC/C=C\CCCCCCCC(=O)NC(COC(=O)NCCCN(CC)CC)C(=O)OCCCCCCCOC(=O)CCCCCCCCC] |
| 747. | 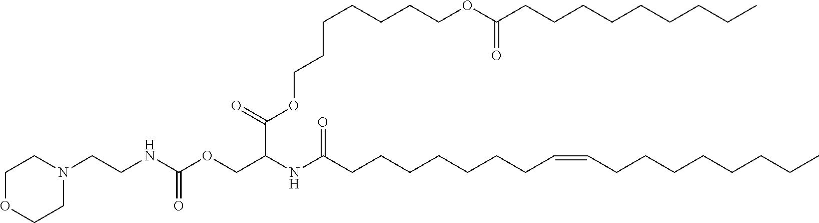 [SMILES: CCCCCCCC/C=C\CCCCCCCC(=O)NC(COC(=O)NCCN1CCOCC1)C(=O)OCCCCCCCOC(=O)CCCCCCCCC] |
| 748. | 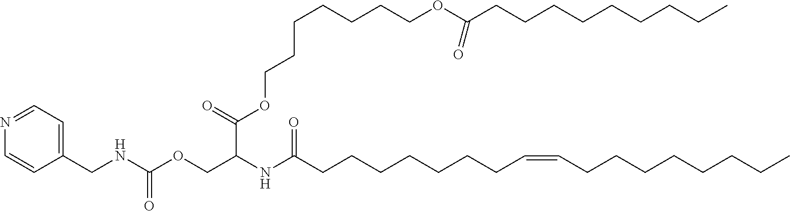 [SMILES: CCCCCCCC/C=C\CCCCCCCC(=O)NC(COC(=O)NCc1ccncc1)C(=O)OCCCCCCCOC(=O)CCCCCCCCC] |
| 749. | 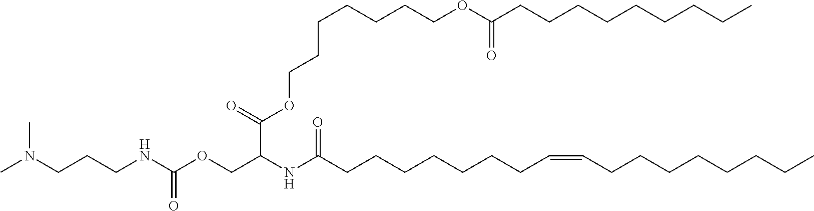 [SMILES: CCCCCCCC/C=C\CCCCCCCC(=O)NC(COC(=O)NCCCN(C)C)C(=O)OCCCCCCCOC(=O)CCCCCCCCC] |
| 750. | 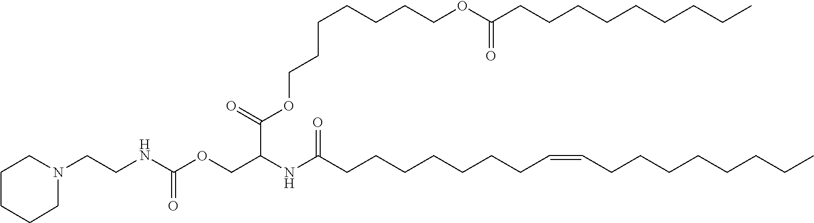 [SMILES: CCCCCCCC/C=C\CCCCCCCC(=O)NC(COC(=O)NCCN1CCCCC1)C(=O)OCCCCCCCOC(=O)CCCCCCCCC] |
| 751. | 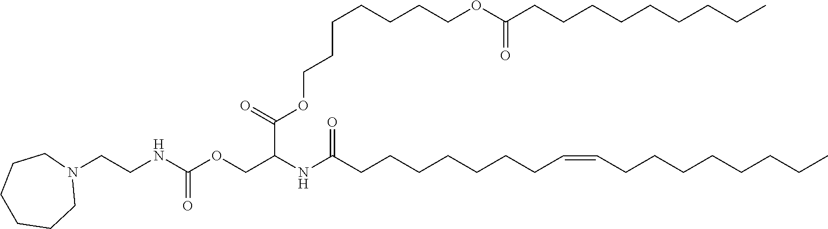 [SMILES: CCCCCCCC/C=C\CCCCCCCC(=O)NC(COC(=O)NCCN1CCCCCC1)C(=O)OCCCCCCCOC(=O)CCCCCCCCC] |
| 752. | 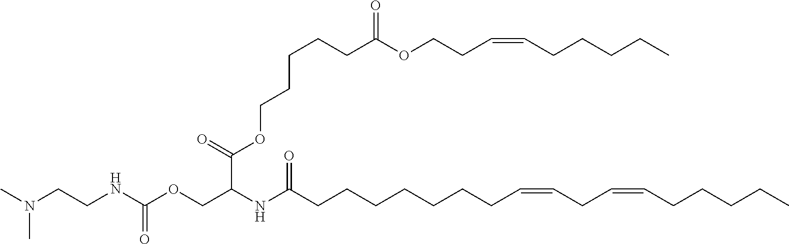 [SMILES: CCCCC/C=C\C/C=C\CCCCCCCC(=O)NC(COC(=O)NCCN(C)C)C(=O)OCCCCCC(=O)OCC/C=C\CCCCC] |
| 753. | 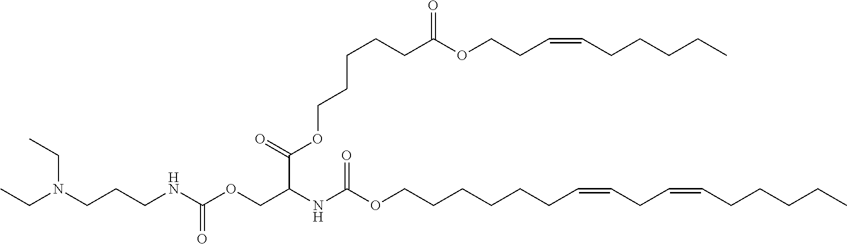 [SMILES: CCCCC/C=C\C/C=C\CCCCCCOC(=O)NC(COC(=O)NCCCN(CC)CC)C(=O)OCCCCCC(=O)OCC/C=C\CCCCC] |
| 754. | 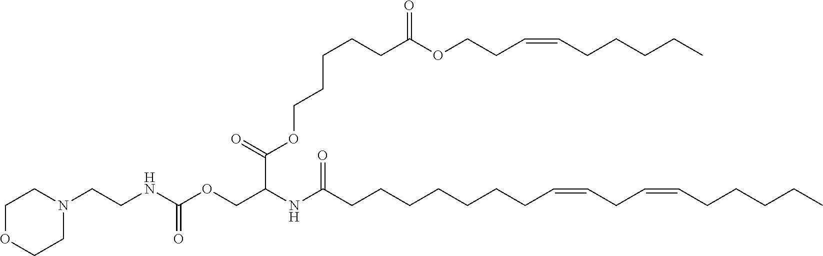 [SMILES: CCCCC/C=C\C/C=C\CCCCCCCC(=O)NC(COC(=O)NCCN1CCOCC1)C(=O)OCCCCCC(=O)OCC/C=C\CCCCC] |
| 755. | 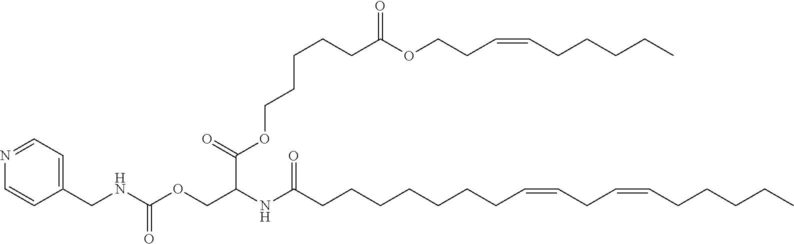 [SMILES: CCCCC/C=C\C/C=C\CCCCCCCC(=O)NC(COC(=O)NCc1ccncc1)C(=O)OCCCCCC(=O)OCC/C=C\CCCCC] |
| 756. | 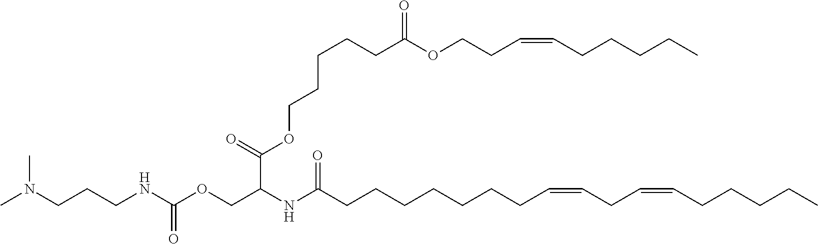 [SMILES: CCCCC/C=C\C/C=C\CCCCCCCC(=O)NC(COC(=O)NCCCN(C)C)C(=O)OCCCCCC(=O)OCC/C=C\CCCCC] |
| 757. | 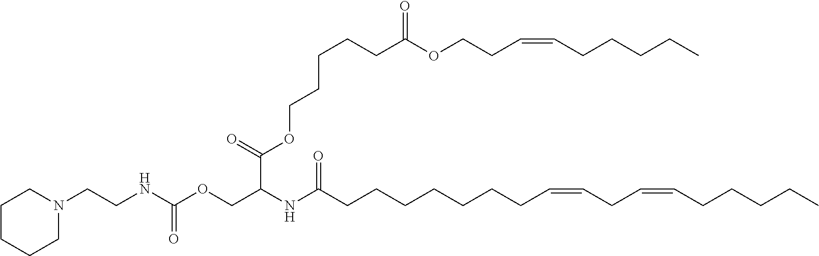 [SMILES: CCCCC/C=C\C/C=C\CCCCCCCC(=O)NC(COC(=O)NCCN1CCCCC1)C(=O)OCCCCCC(=O)OCC/C=C\CCCCC] |
| 758. | 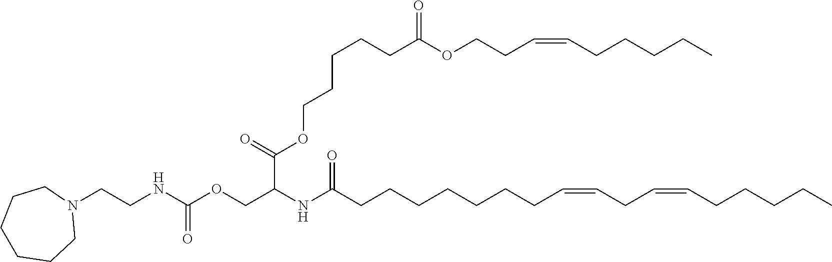 [SMILES: CCCCC/C=C\C/C=C\CCCCCCCC(=O)NC(COC(=O)NCCN1CCCCCC1)C(=O)OCCCCCC(=O)OCC/C=C\CCCCC] |
| 759. | 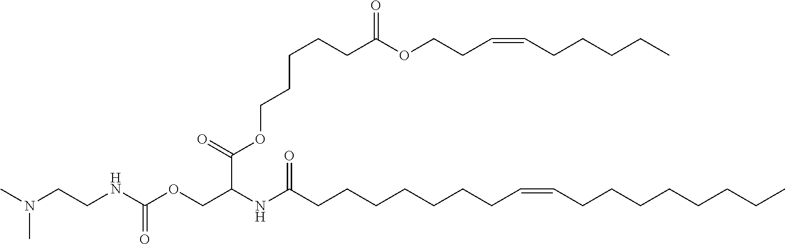 [SMILES: CCCCC/C=C\CCOC(=O)CCCCCOC(=O)C(COC(=O)NCCN(C)C)NC(=O)CCCCCCC/C=C\CCCCCCCC] |
| 760. | 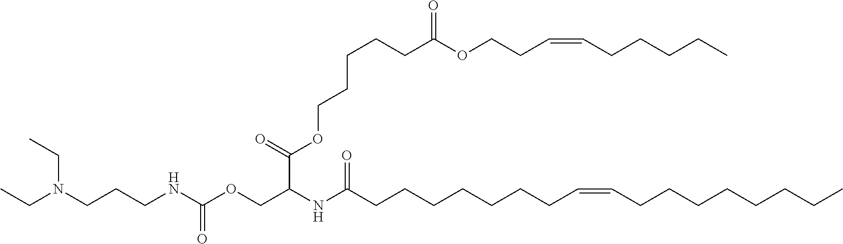 [SMILES: CCCCC/C=C\CCOC(=O)CCCCCOC(=O)C(COC(=O)NCCCN(CC)CC)NC(=O)CCCCCCC/C=C\CCCCCCCC] |
| 761. | 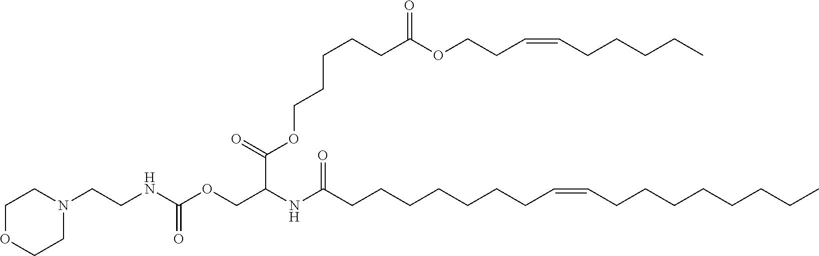 [SMILES: CCCCC/C=C\CCOC(=O)CCCCCOC(=O)C(COC(=O)NCCN1CCOCC1)NC(=O)CCCCCCC/C=C\CCCCCCCC] |
| 762. | 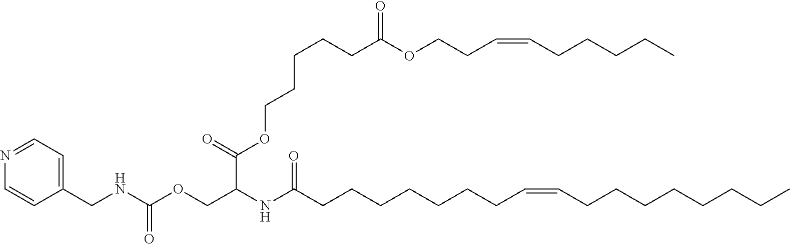 [SMILES: CCCCC/C=C\CCOC(=O)CCCCCOC(=O)C(COC(=O)NCc1ccncc1)NC(=O)CCCCCCC/C=C\CCCCCCCC] |
| 763. | 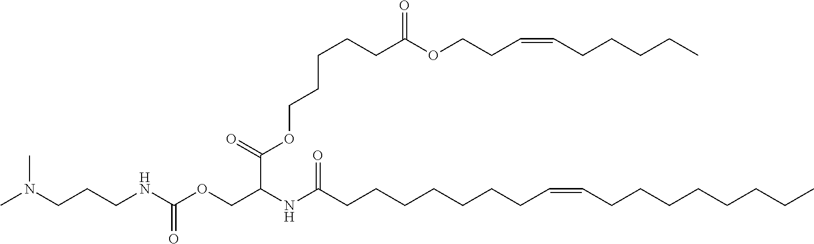 [SMILES: CCCCC/C=C\CCOC(=O)CCCCCOC(=O)C(COC(=O)NCCCN(C)C)NC(=O)CCCCCCC/C=C\CCCCCCCC] |
| 764. | 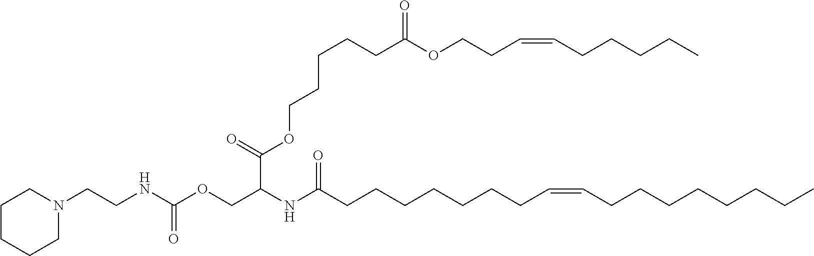 [SMILES: CCCCC/C=C\CCOC(=O)CCCCCOC(=O)C(COC(=O)NCCN1CCCCC1)NC(=O)CCCCCCC/C=C\CCCCCCCC] |
| 765. | 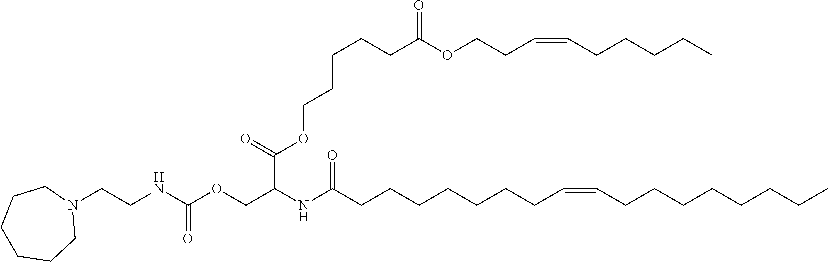 [SMILES: CCCCC/C=C\CCOC(=O)CCCCCOC(=O)C(COC(=O)NCCN1CCCCCC1)NC(=O)CCCCCCC/C=C\CCCCCCCC] |
| 766. | 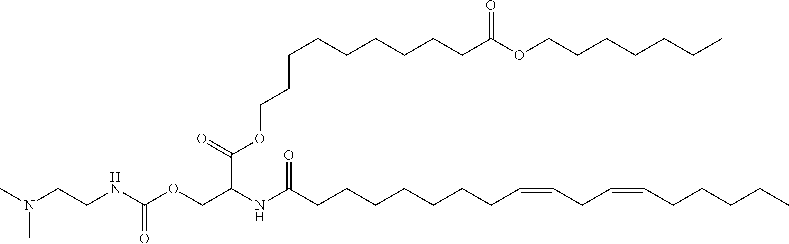 [SMILES: CCCCC/C=C\C/C=C\CCCCCCCC(=O)NC(COC(=O)NCCN(C)C)C(=O)OCCCCCCCCCC(=O)OCCCCCCC] |
| 767. | 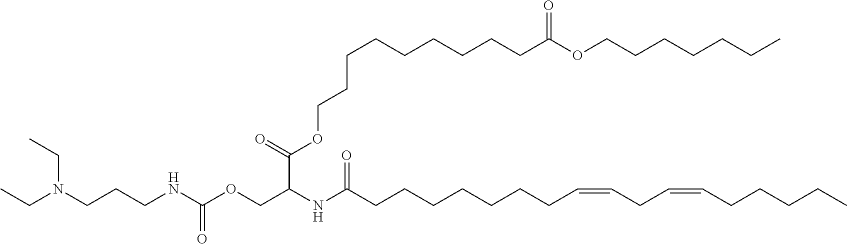 [SMILES: CCCCC/C=C\C/C=C\CCCCCCCC(=O)NC(COC(=O)NCCCN(CC)CC)C(=O)OCCCCCCCCCC(=O)OCCCCCCC] |
| 768. | 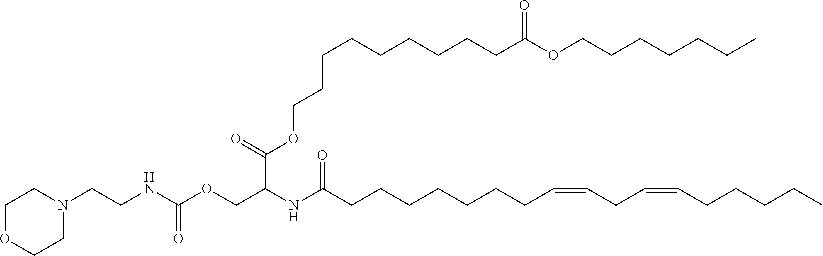 [SMILES: CCCCC/C=C\C/C=C\CCCCCCCC(=O)NC(COC(=O)NCCN1CCOCC1)C(=O)OCCCCCCCCCC(=O)OCCCCCCC] |
| 769. | 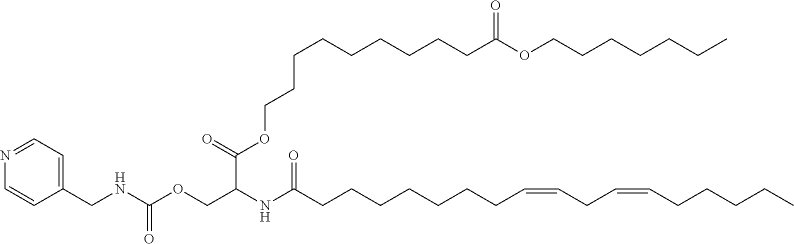 [SMILES: CCCCC/C=C\C/C=C\CCCCCCCC(=O)NC(COC(=O)NCc1ccncc1)C(=O)OCCCCCCCCCC(=O)OCCCCCCC] |
| 770. | 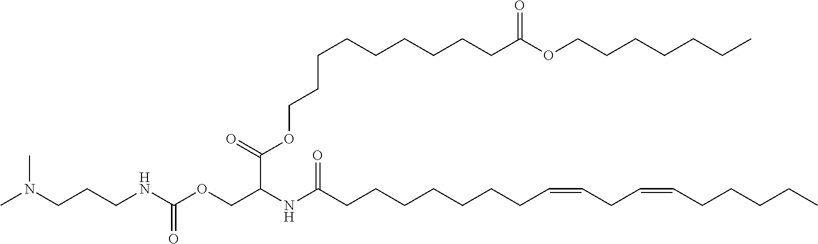 [SMILES: CCCCC/C=C\C/C=C\CCCCCCCC(=O)NC(COC(=O)NCCCN(C)C)C(=O)OCCCCCCCCCC(=O)OCCCCCCC] |
| 771. | 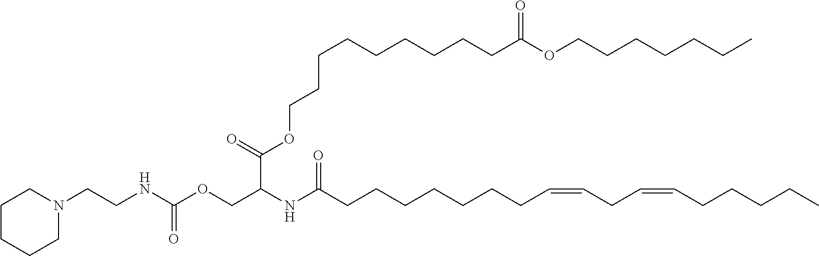 [SMILES: CCCCC/C=C\C/C=C\CCCCCCCC(=O)NC(COC(=O)NCCN1CCCCC1)C(=O)OCCCCCCCCCC(=O)OCCCCCCC] |
| 772. | 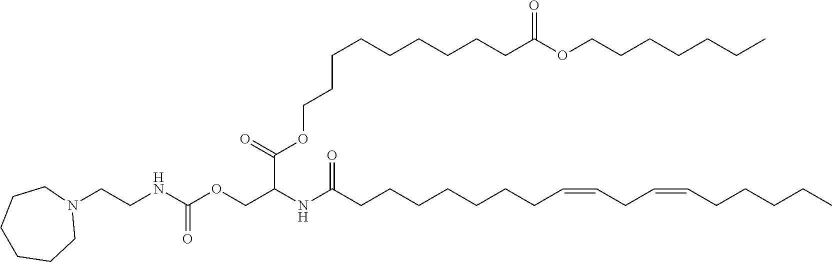 [SMILES: CCCCC/C=C\C/C=C\CCCCCCCC(=O)NC(COC(=O)NCCN1CCCCCC1)C(=O)OCCCCCCCCCC(=O)OCCCCCCC] |
| 773. | 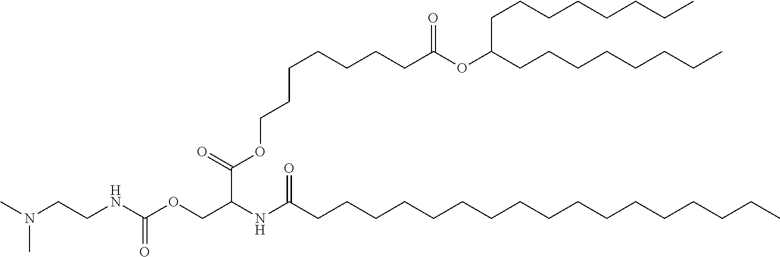 [SMILES: CCCCCCCCCCCCCCCCCC(=O)NC(COC(=O)NCCN(C)C)C(=O)OCCCCCCCC(=O)OC(CCCCCCCC)CCCCCCCC] |
| 774. | 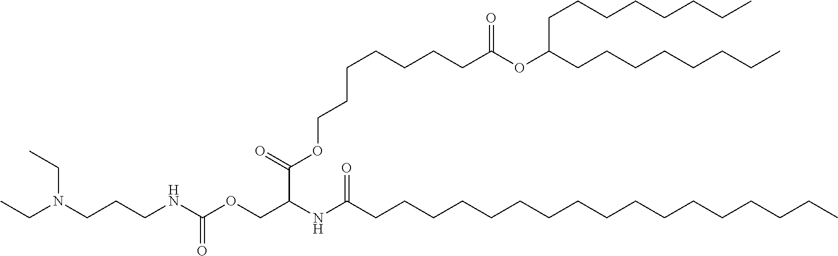 [SMILES: CCCCCCCCCCCCCCCCCC(=O)NC(COC(=O)NCCCN(CC)CC)C(=O)OCCCCCCCC(=O)OC(CCCCCCCC)CCCCCCCC] |
| 775. | 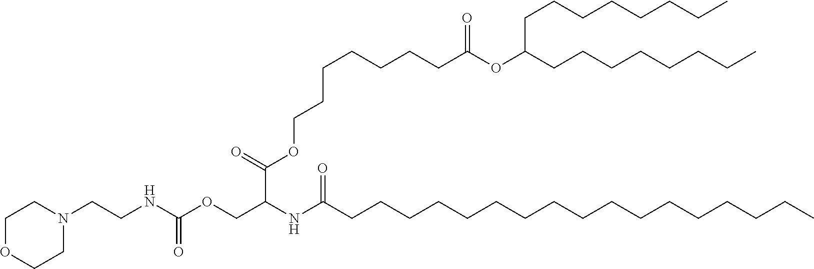 [SMILES: CCCCCCCCCCCCCCCCCC(=O)NC(COC(=O)NCCN1CCOCC1)C(=O)OCCCCCCCC(=O)OC(CCCCCCCC)CCCCCCCC] |
| 776. | 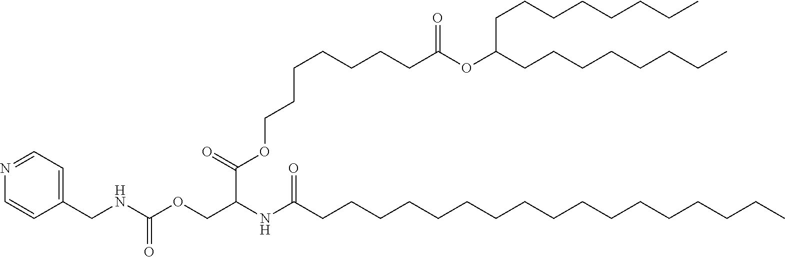 [SMILES: CCCCCCCCCCCCCCCCCC(=O)NC(COC(=O)NCc1ccncc1)C(=O)OCCCCCCCC(=O)OC(CCCCCCCC)CCCCCCCC] |
| 777. | 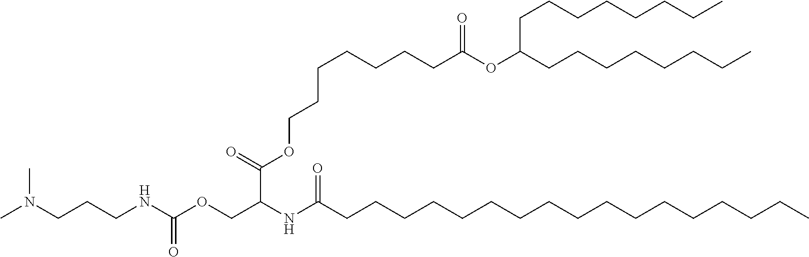 [SMILES: CCCCCCCCCCCCCCCCCC(=O)NC(COC(=O)NCCCN(C)C)C(=O)OCCCCCCCC(=O)OC(CCCCCCCC)CCCCCCCC] |
| 778. | 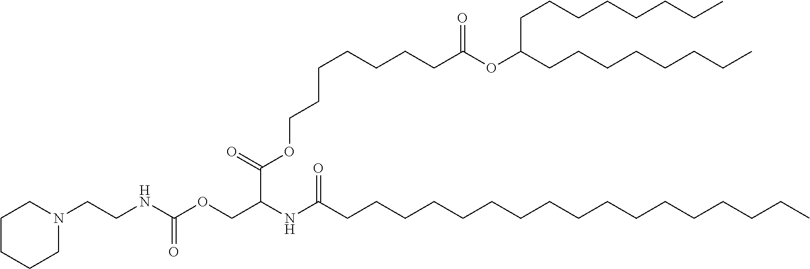 [SMILES: CCCCCCCCCCCCCCCCCC(=O)NC(COC(=O)NCCN1CCCCC1)C(=O)OCCCCCCCC(=O)OC(CCCCCCCC)CCCCCCCC] |
| 779. | 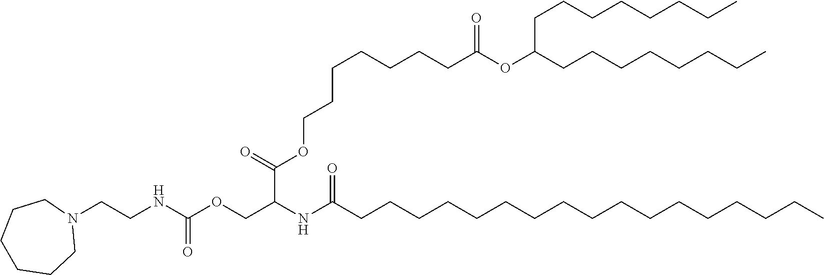 [SMILES: CCCCCCCCCCCCCCCCCC(=O)NC(COC(=O)NCCN1CCCCCC1)C(=O)OCCCCCCCC(=O)OC(CCCCCCCC)CCCCCCCC] |
| 780. | 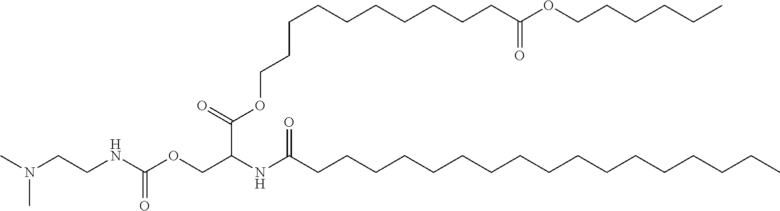 [SMILES: CCCCCCCCCCCCCCCCCC(=O)NC(COC(=O)NCCN(C)C)C(=O)OCCCCCCCCCCC(=O)OCCCCCC] |
| 781. | 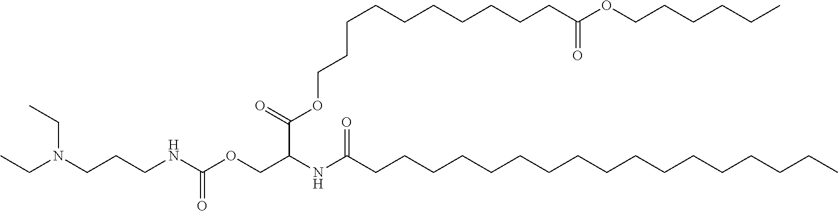 |
| 782. | 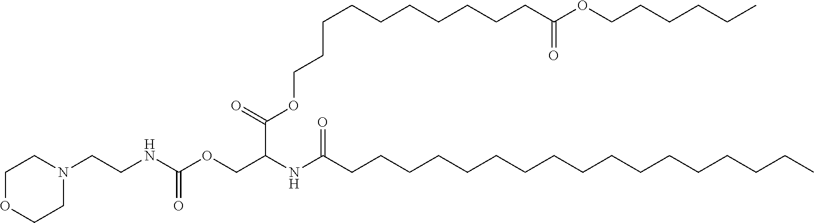 [SMILES: CCCCCCCCCCCCCCCCCC(=O)NC(COC(=O)NCCN1CCOCC1)C(=O)OCCCCCCCCCCC(=O)OCCCCCC] |
| 783. | 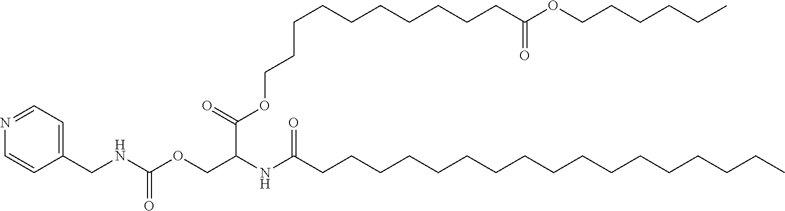 [SMILES: CCCCCCCCCCCCCCCCCC(=O)NC(COC(=O)NCc1ccncc1)C(=O)OCCCCCCCCCCC(=O)OCCCCCC] |
| 784. | 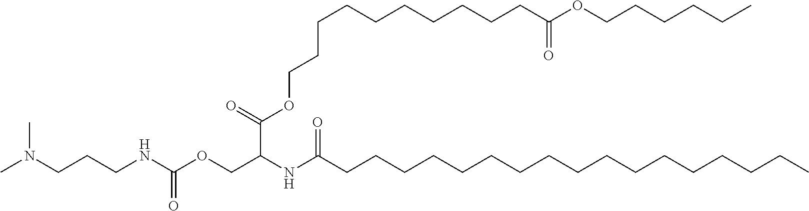 [SMILES: CCCCCCCCCCCCCCCCCC(=O)NC(COC(=O)NCCCN(C)C)C(=O)OCCCCCCCCCCC(=O)OCCCCCC] |
| 785. | 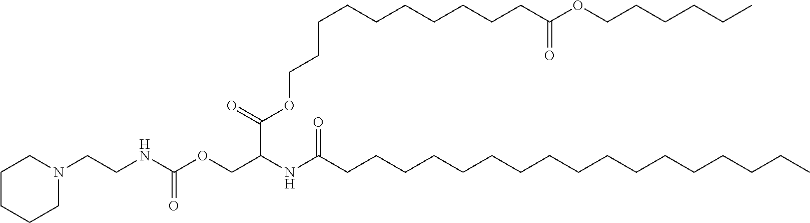 [SMILES: CCCCCCCCCCCCCCCCCC(=O)NC(COC(=O)NCCN1CCCCC1)C(=O)OCCCCCCCCCCC(=O)OCCCCCC] |
| 786. | 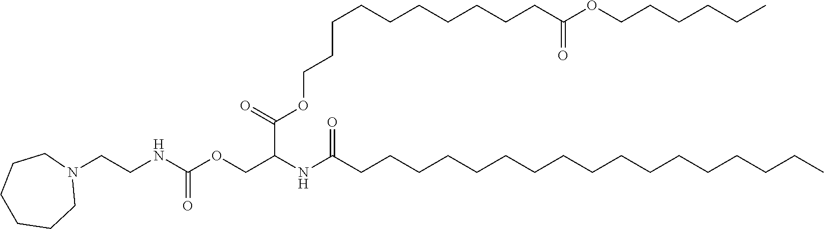 [SMILES: CCCCCCCCCCCCCCCCCC(=O)NC(COC(=O)NCCN1CCCCCC1)C(=O)OCCCCCCCCCCC(=O)OCCCCCC] |
| 787. | 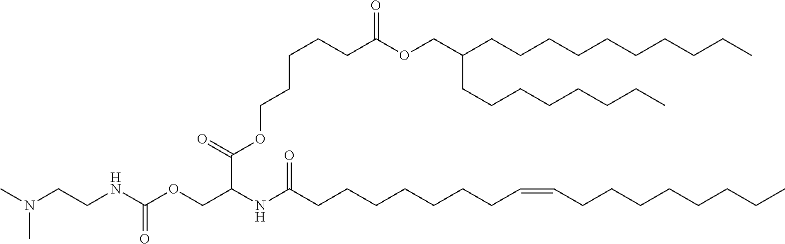 [SMILES: CCCCCCCC/C=C\CCCCCCCC(=O)NC(COC(=O)NCCN(C)C)C(=O)OCCCCCC(=O)OCC(CCCCCCCC)CCCCCCCCCC] |
| 788. | 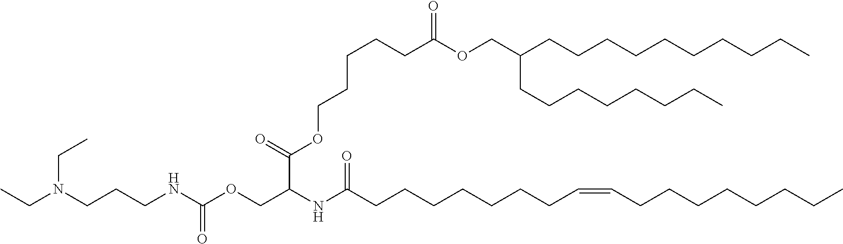 [SMILES: CCCCCCCC/C=C\CCCCCCCC(=O)NC(COC(=O)NCCCN(CC)CC)C(=O)OCCCCCC(=O)OCC(CCCCCCCC)CCCCCCCCCC] |
| 789. | 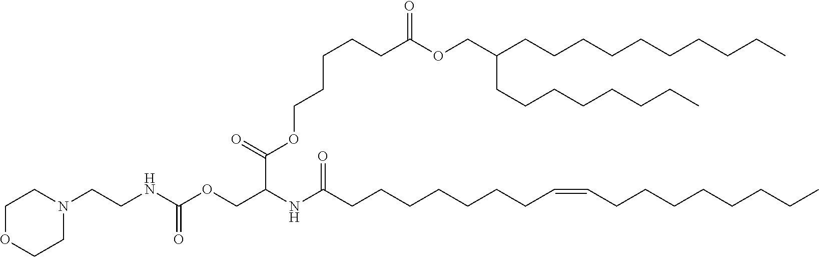 [SMILES: CCCCCCCC/C=C\CCCCCCCC(=O)NC(COC(=O)NCCN1CCOCC1)C(=O)OCCCCCC(=O)OCC(CCCCCCCC)CCCCCCCCCC] |
| 790. | 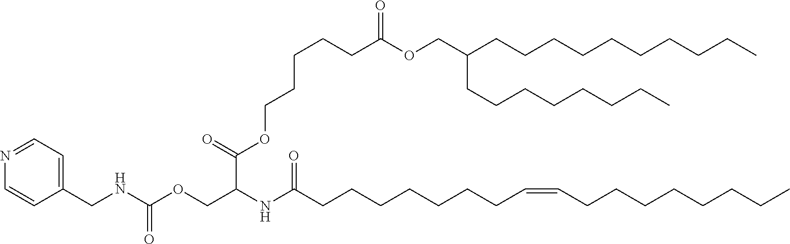 [SMILES: CCCCCCCC/C=C\CCCCCCCC(=O)NC(COC(=O)NCc1ccncc1)C(=O)OCCCCCC(=O)OCC(CCCCCCCC)CCCCCCCCCC] |
| 791. | 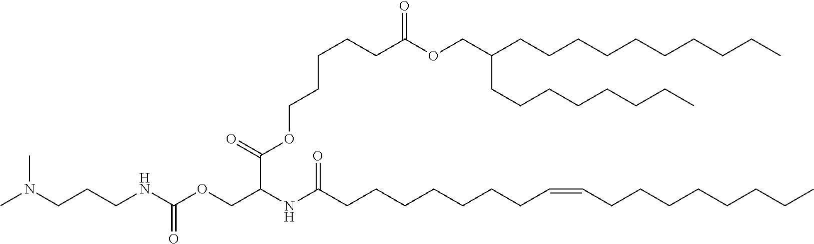 [SMILES: CCCCCCCC/C=C\CCCCCCCC(=O)NC(COC(=O)NCCCN(C)C)C(=O)OCCCCCC(=O)OCC(CCCCCCCC)CCCCCCCCCC] |
| 792. | 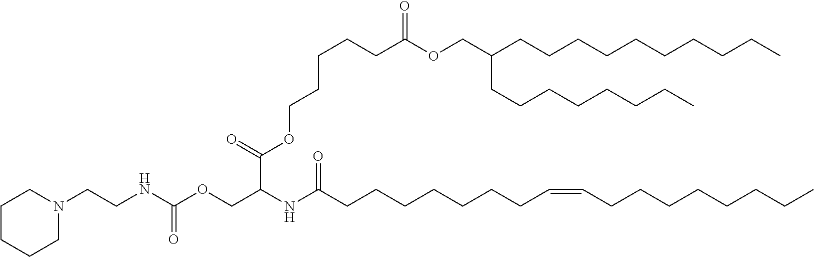 [SMILES: CCCCCCCC/C=C\CCCCCCCC(=O)NC(COC(=O)NCCN1CCCCC1)C(=O)OCCCCCC(=O)OCC(CCCCCCCC)CCCCCCCCCC] |
| 793. | 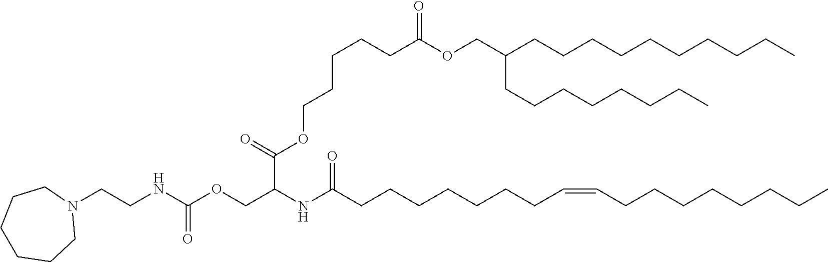 [SMILES: CCCCCCCC/C=C\CCCCCCCC(=O)NC(COC(=O)NCCN1CCCCCC1)C(=O)OCCCCCC(=O)OCC(CCCCCCCC)CCCCCCCCCC] |
| 794. | 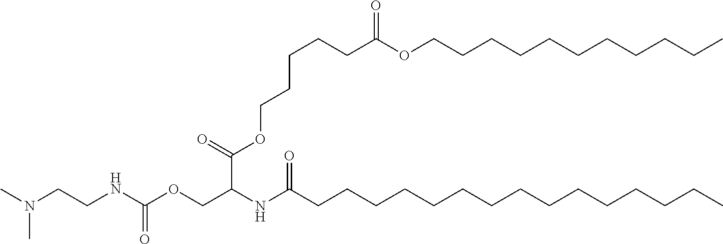 [SMILES: CCCCCCCCCCCCCCCC(=O)NC(COC(=O)NCCN(C)C)C(=O)OCCCCCC(=O)OCCCCCCCCCCC] |
| 795. | 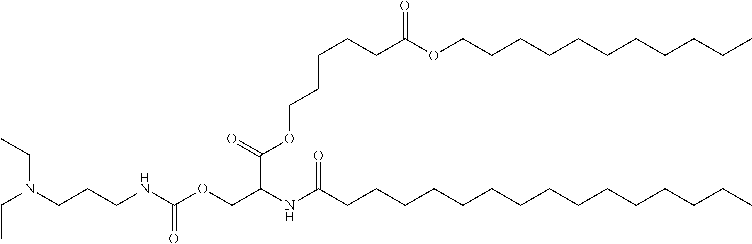 [SMILES: CCCCCCCCCCCCCCCC(=O)NC(COC(=O)NCCCN(CC)CC)C(=O)OCCCCCC(=O)OCCCCCCCCCCC] |
| 796. | 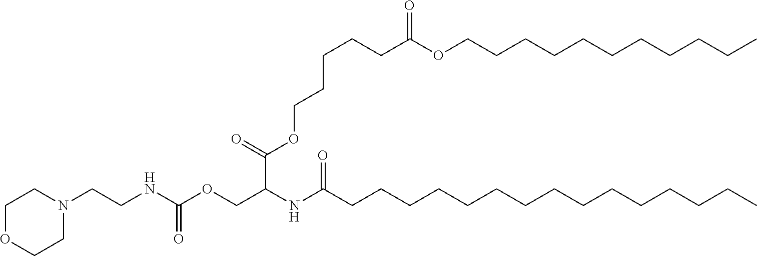 [SMILES: CCCCCCCCCCCCCCCC(=O)NC(COC(=O)NCCN1CCOCC1)C(=O)OCCCCCC(=O)OCCCCCCCCCCC] |
| 797. | 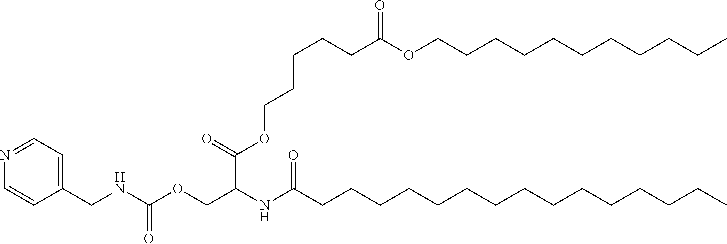 [SMILES: CCCCCCCCCCCCCCCC(=O)NC(COC(=O)NCc1ccncc1)C(=O)OCCCCCC(=O)OCCCCCCCCCCC] |
| 798. | 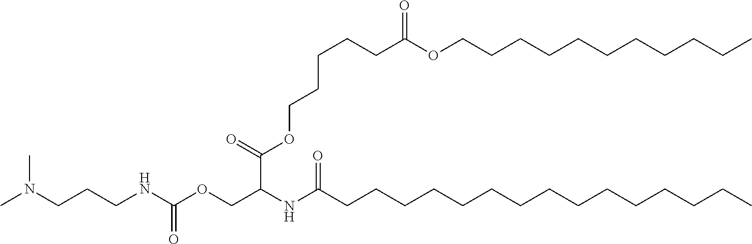 [SMILES: CCCCCCCCCCCCCCCC(=O)NC(COC(=O)NCCCN(C)C)C(=O)OCCCCCC(=O)OCCCCCCCCCCC] |
| 799. | 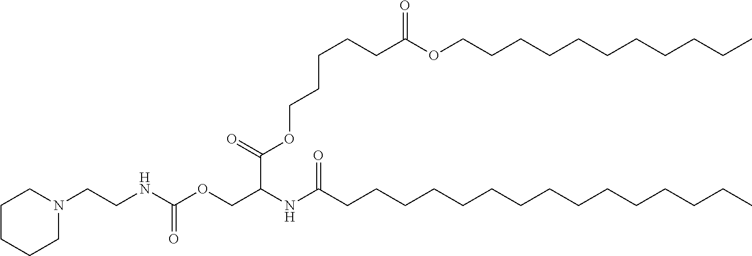 [SMILES: CCCCCCCCCCCCCCCC(=O)NC(COC(=O)NCCN1CCCCC1)C(=O)OCCCCCC(=O)OCCCCCCCCCCC] |
| 800. | 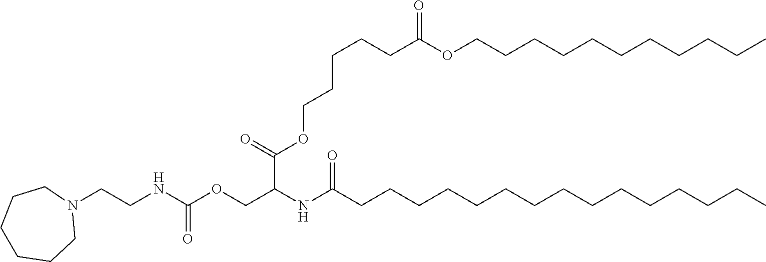 [SMILES: CCCCCCCCCCCCCCCC(=O)NC(COC(=O)NCCN1CCCCCC1)C(=O)OCCCCCC(=O)OCCCCCCCCCCC] |
| 801. | 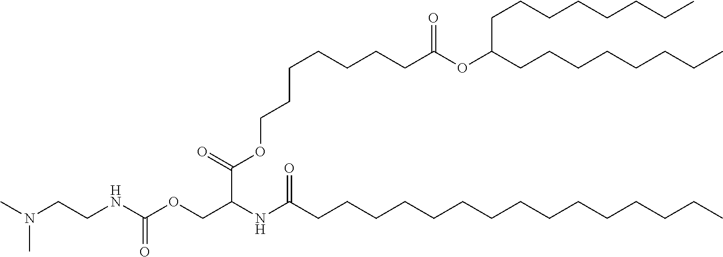 [SMILES: CCCCCCCCCCCCCCCC(=O)NC(COC(=O)NCCN(C)C)C(=O)OCCCCCCCC(=O)OC(CCCCCCCC)CCCCCCCC] |
| 802. | 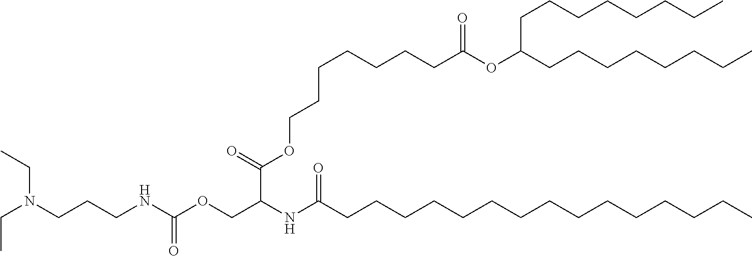 [SMILES: CCCCCCCCCCCCCCCC(=O)NC(COC(=O)NCCCN(CC)CC)C(=O)OCCCCCCCC(=O)OC(CCCCCCCC)CCCCCCCC] |
| 803. | 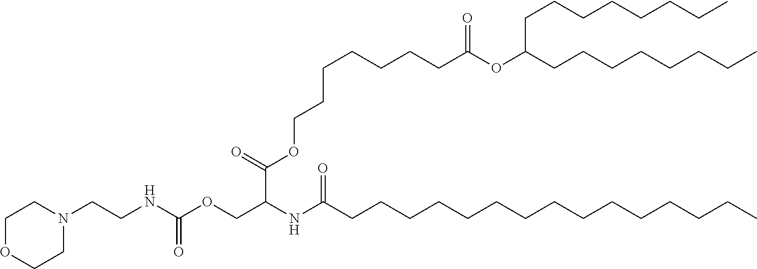 [SMILES: CCCCCCCCCCCCCCCC(=O)NC(COC(=O)NCCN1CCOCC1)C(=O)OCCCCCCCC(=O)OC(CCCCCCCC)CCCCCCCC] |
| 804. | 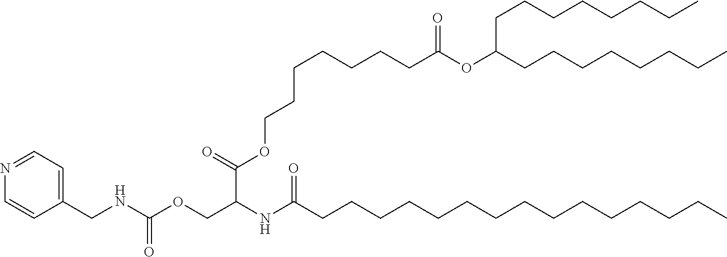 [SMILES: CCCCCCCCCCCCCCCC(=O)NC(COC(=O)NCc1ccncc1)C(=O)OCCCCCCCC(=O)OC(CCCCCCCC)CCCCCCCC] |
| 805. | 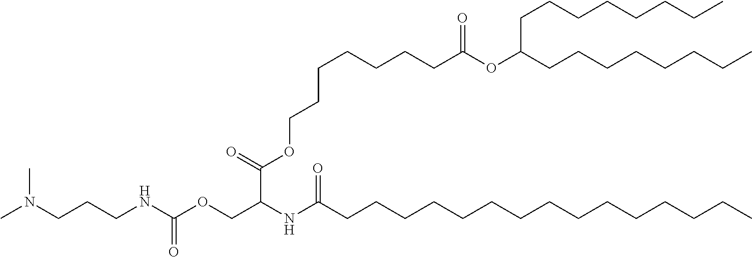 [SMILES: CCCCCCCCCCCCCCCC(=O)NC(COC(=O)NCCCN(C)C)C(=O)OCCCCCCCC(=O)OC(CCCCCCCC)CCCCCCCC] |
| 806. | 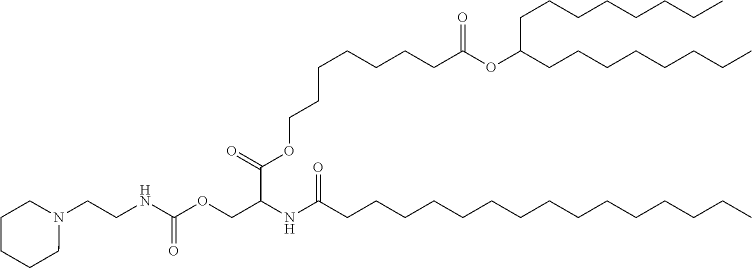 [SMILES: CCCCCCCCCCCCCCCC(=O)NC(COC(=O)NCCN1CCCCC1)C(=O)OCCCCCCCC(=O)OC(CCCCCCCC)CCCCCCCC] |
| 807. | 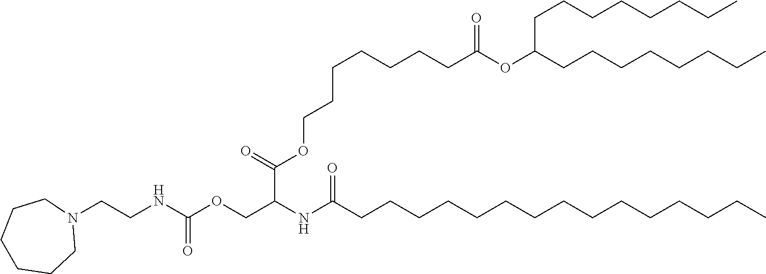 [SMILES: CCCCCCCCCCCCCCCC(=O)NC(COC(=O)NCCN1CCCCCC1)C(=O)OCCCCCCCC(=O)OC(CCCCCCCC)CCCCCCCC] |
| 808. | 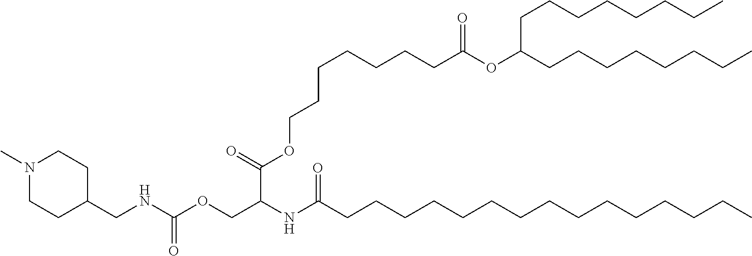 [SMILES: CCCCCCCCCCCCCCCC(=O)NC(COC(=O)NCC1CCN(C)CC1)C(=O)OCCCCCCCC(=O)OC(CCCCCCCC)CCCCCCCC] |
| 809. | 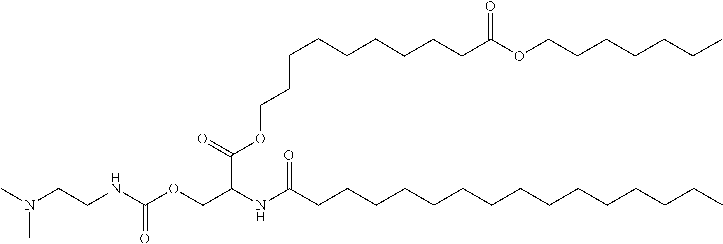 [SMILES: CCCCCCCCCCCCCCCC(=O)NC(COC(=O)NCCN(C)C)C(=O)OCCCCCCCCCC(=O)OCCCCCCC] |
| 810. | 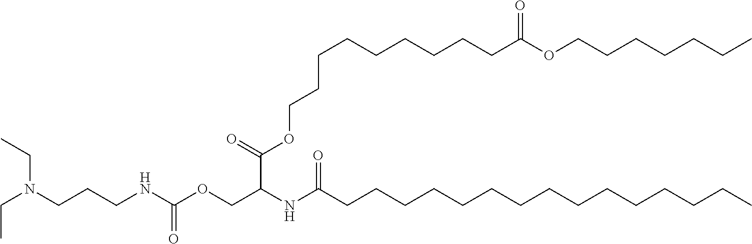 [SMILES: CCCCCCCCCCCCCCCC(=O)NC(COC(=O)NCCCN(CC)CC)C(=O)OCCCCCCCCCC(=O)OCCCCCCC] |
| 811. | 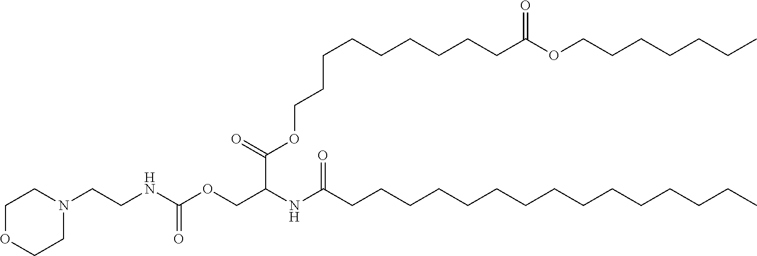 [SMILES: CCCCCCCCCCCCCCCC(=O)NC(COC(=O)NCCN1CCOCC1)C(=O)OCCCCCCCCCC(=O)OCCCCCCC] |
| 812. | 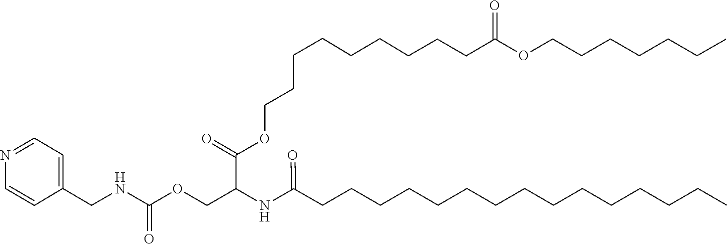 [SMILES: CCCCCCCCCCCCCCCC(=O)NC(COC(=O)NCc1ccncc1)C(=O)OCCCCCCCCCC(=O)OCCCCCCC] |
| 813. | 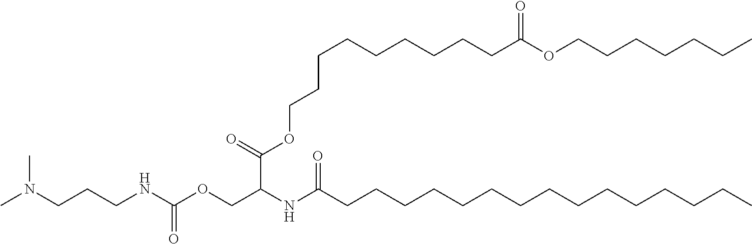 [SMILES: CCCCCCCCCCCCCCCC(=O)NC(COC(=O)NCCCN(C)C)C(=O)OCCCCCCCCCC(=O)OCCCCCCC] |
| 814. | 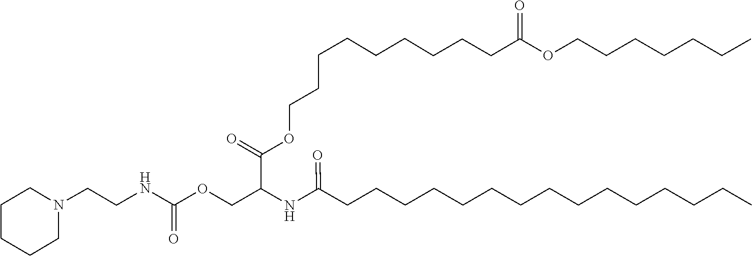 [SMILES: CCCCCCCCCCCCCCCC(=O)NC(COC(=O)NCCN1CCCCC1)C(=O)OCCCCCCCCCC(=O)OCCCCCCC] |
| 815. | 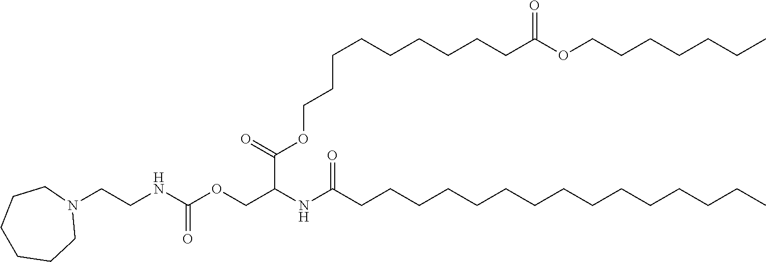 [SMILES: CCCCCCCCCCCCCCCC(=O)NC(COC(=O)NCCN1CCCCCC1)C(=O)OCCCCCCCCCC(=O)OCCCCCCC] |
| 816. | 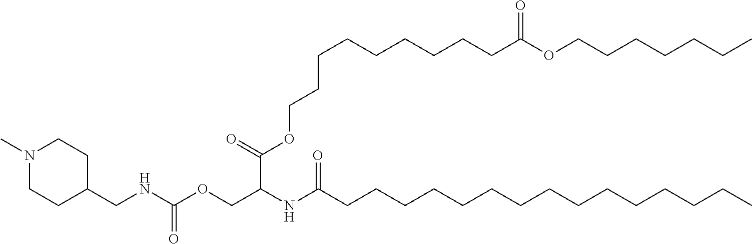 [SMILES: CCCCCCCCCCCCCCCC(=O)NC(COC(=O)NCC1CCN(C)CC1)C(=O)OCCCCCCCCCC(=O)OCCCCCCC] |
| 817. | 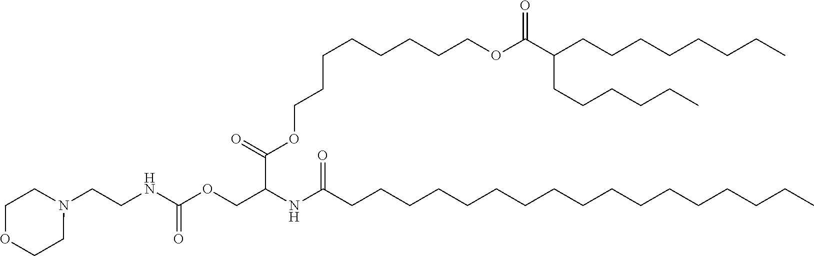 [SMILES: CCCCCCCCCCCCCCCCCC(=O)NC(COC(=O)NCCN1CCOCC1)C(=O)OCCCCCCCCOC(=O)C(CCCCCC)CCCCCCCC] |
| 818. | 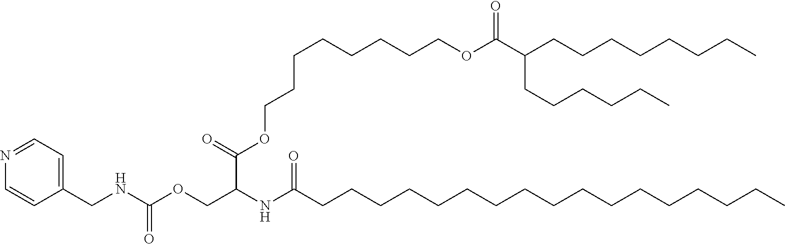 [SMILES: CCCCCCCCCCCCCCCCCC(=O)NC(COC(=O)NCc1ccncc1)C(=O)OCCCCCCCCOC(=O)C(CCCCCC)CCCCCCCC] |
| 819. | 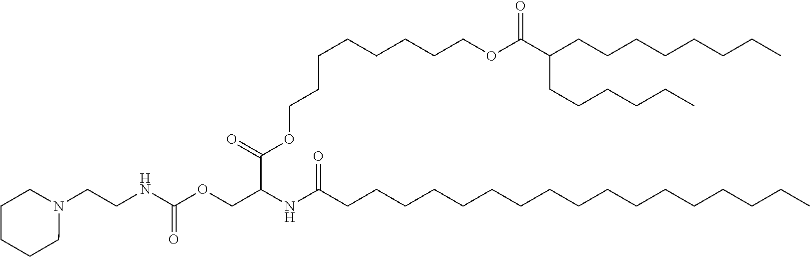 [SMILES: CCCCCCCCCCCCCCCCCC(=O)NC(COC(=O)NCCN1CCCCC1)C(=O)OCCCCCCCCOC(=O)C(CCCCCC)CCCCCCCC] |
| 820. | 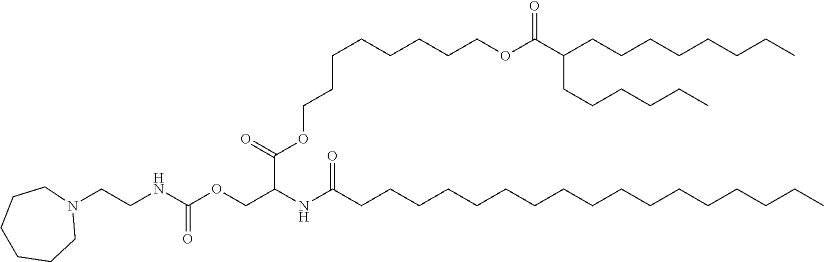 [SMILES: CCCCCCCCCCCCCCCCCC(=O)NC(COC(=O)NCCN1CCCCCC1)C(=O)OCCCCCCCCOC(=O)C(CCCCCC)CCCCCCCC] |
| 821. | 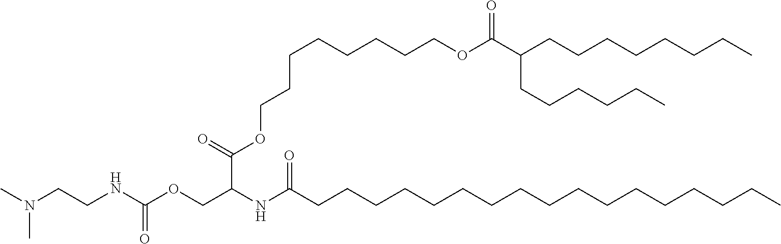 [SMILES: CCCCCCCCCCCCCCCCCC(=O)NC(COC(=O)NCCN(C)C)C(=O)OCCCCCCCCOC(=O)C(CCCCCC)CCCCCCCC] |
| 822. | 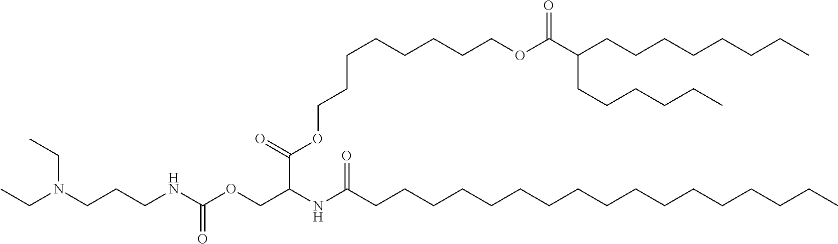 [SMILES: CCCCCCCCCCCCCCCCCC(=O)NC(COC(=O)NCCCN(CC)CC)C(=O)OCCCCCCCCOC(=O)C(CCCCCC)CCCCCCCC] |
| 823. | 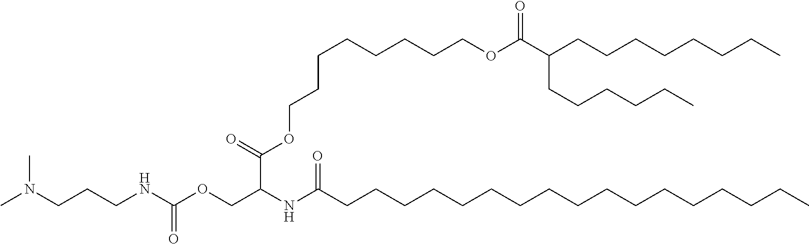 [SMILES: CCCCCCCCCCCCCCCCCC(=O)NC(COC(=O)NCCCN(C)C)C(=O)OCCCCCCCCOC(=O)C(CCCCCC)CCCCCCCC] |
| 824. | 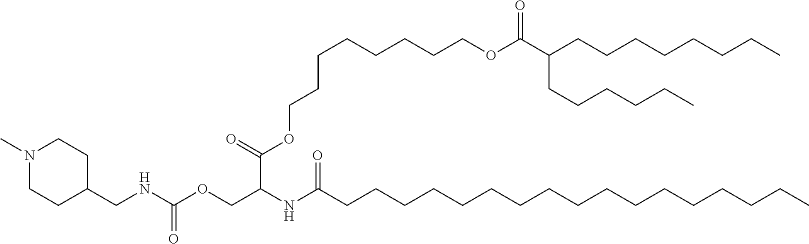 [SMILES: CCCCCCCCCCCCCCCCCC(=O)NC(COC(=O)NCC1CCN(C)CC1)C(=O)OCCCCCCCCOC(=O)C(CCCCCC)CCCCCCCC] |
| 825. | 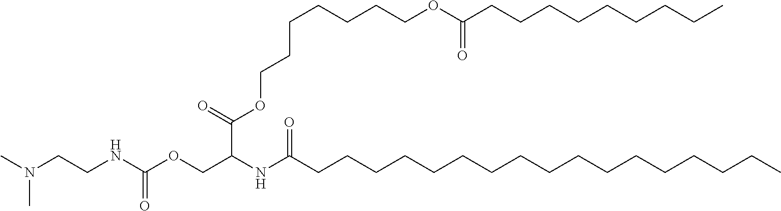 [SMILES: CCCCCCCCCCCCCCCCCC(=O)NC(COC(=O)NCCN(C)C)C(=O)OCCCCCCCOC(=O)CCCCCCCCC] |
| 826. | 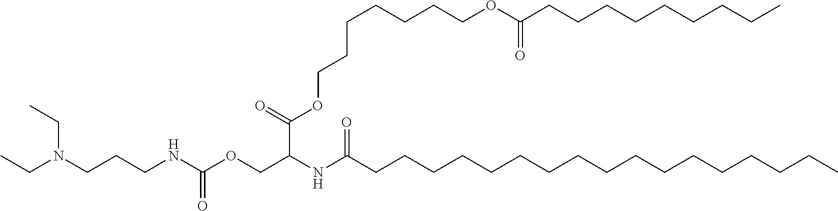 |
| 827. | 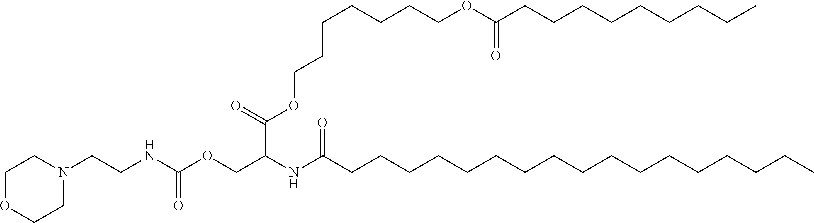 [SMILES: CCCCCCCCCCCCCCCCCC(=O)NC(COC(=O)NCCN1CCOCC1)C(=O)OCCCCCCCOC(=O)CCCCCCCCC] |
| 828. | 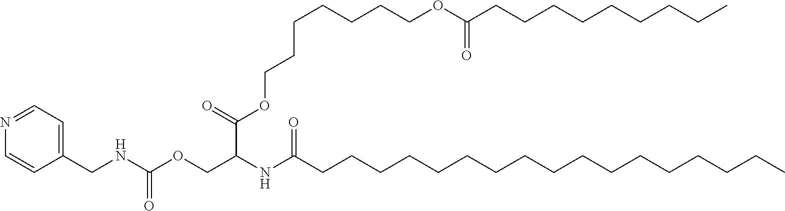 [SMILES: CCCCCCCCCCCCCCCCCC(=O)NC(COC(=O)NCc1ccncc1)C(=O)OCCCCCCCOC(=O)CCCCCCCCC] |
| 829. | 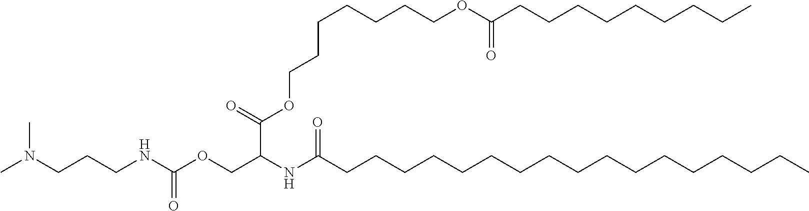 [SMILES: CCCCCCCCCCCCCCCCCC(=O)NC(COC(=O)NCCCN(C)C)C(=O)OCCCCCCCOC(=O)CCCCCCCCC] |
| 830. | 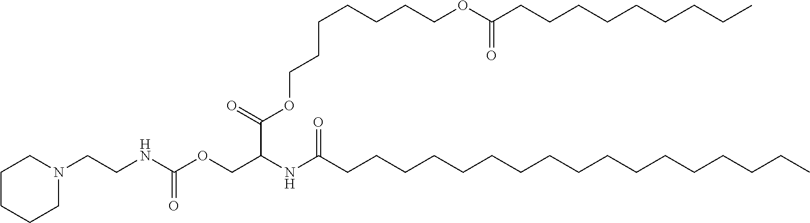 [SMILES: CCCCCCCCCCCCCCCCCC(=O)NC(COC(=O)NCCN1CCCCC1)C(=O)OCCCCCCCOC(=O)CCCCCCCCC] |
| 831. | 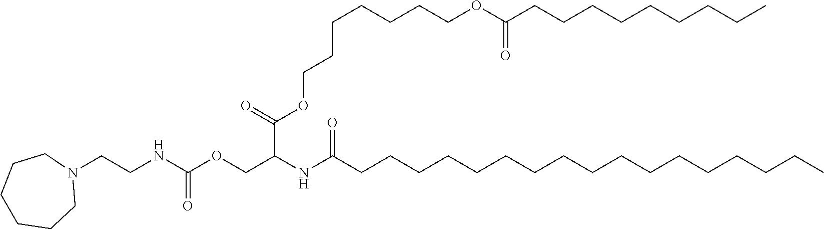 [SMILES: CCCCCCCCCCCCCCCCCC(=O)NC(COC(=O)NCCN1CCCCCC1)C(=O)OCCCCCCCOC(=O)CCCCCCCCC] |
| 832. | 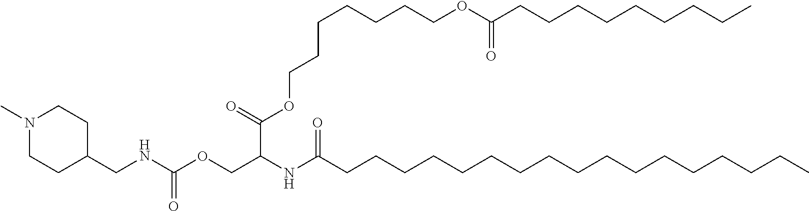 [SMILES: CCCCCCCCCCCCCCCCCC(=O)NC(COC(=O)NCC1CCN(C)CC1)C(=O)OCCCCCCCOC(=O)CCCCCCCCC] |
| 833. | 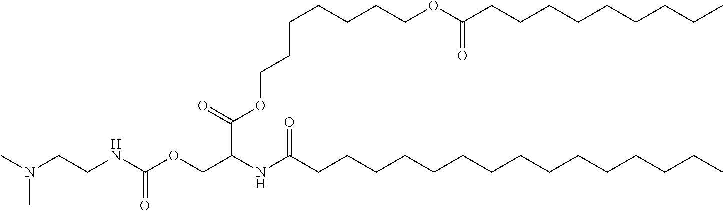 [SMILES: CCCCCCCCCCCCCCCC(=O)NC(COC(=O)NCCN(C)C)C(=O)OCCCCCCCOC(=O)CCCCCCCCC] |
| 834. | 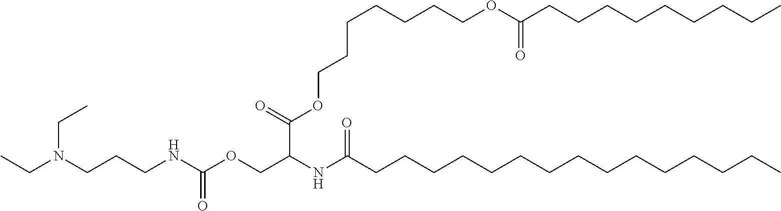 [SMILES: CCCCCCCCCCCCCCCC(=O)NC(COC(=O)NCCCN(CC)CC)C(=O)OCCCCCCCOC(=O)CCCCCCCCC] |
| 835. | 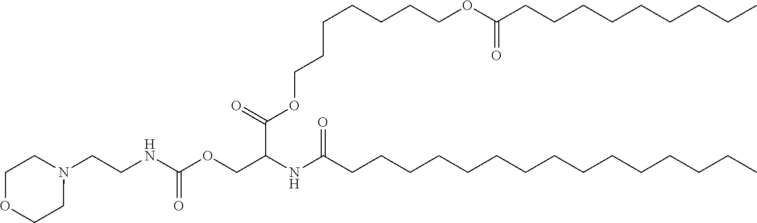 [SMILES: CCCCCCCCCCCCCCCC(=O)NC(COC(=O)NCCN1CCOCC1)C(=O)OCCCCCCCOC(=O)CCCCCCCCC] |
| 836. | 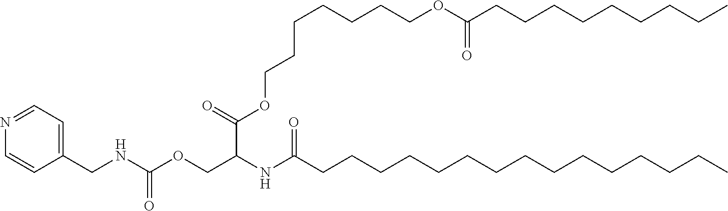 [SMILES: CCCCCCCCCCCCCCCC(=O)NC(COC(=O)NCc1ccncc1)C(=O)OCCCCCCCOC(=O)CCCCCCCCC] |
| 837. | 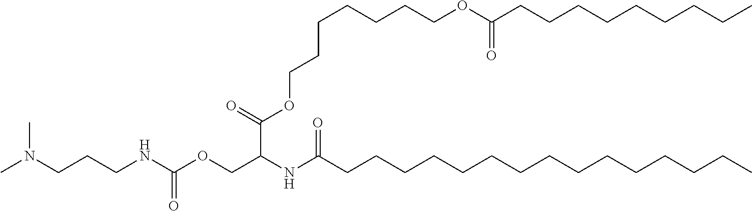 [SMILES: CCCCCCCCCCCCCCCC(=O)NC(COC(=O)NCCCN(C)C)C(=O)OCCCCCCCOC(=O)CCCCCCCCC] |
| 838. | 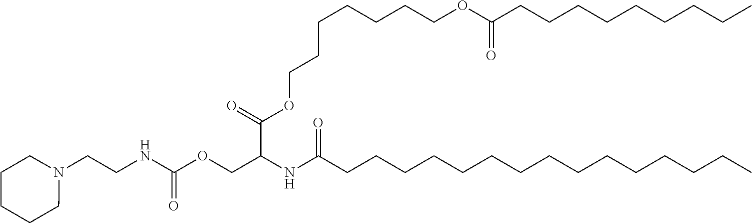 [SMILES: CCCCCCCCCCCCCCCC(=O)NC(COC(=O)NCCN1CCCCC1)C(=O)OCCCCCCCOC(=O)CCCCCCCCC] |
| 839. | 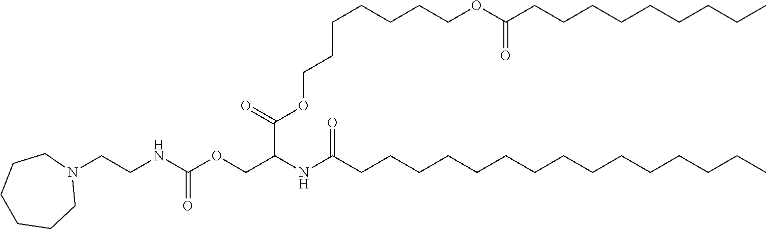 [SMILES: CCCCCCCCCCCCCCCC(=O)NC(COC(=O)NCCN1CCCCCC1)C(=O)OCCCCCCCOC(=O)CCCCCCCCC] |
| 840. | 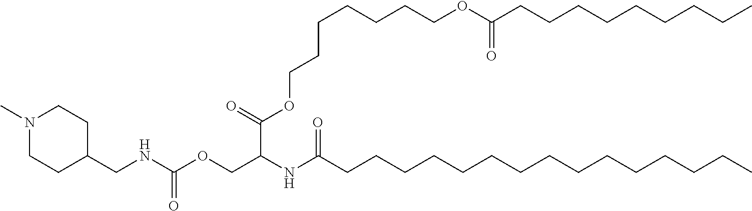 [SMILES: CCCCCCCCCCCCCCCC(=O)NC(COC(=O)NCC1CCN(C)CC1)C(=O)OCCCCCCCOC(=O)CCCCCCCCC] |
| 841. | 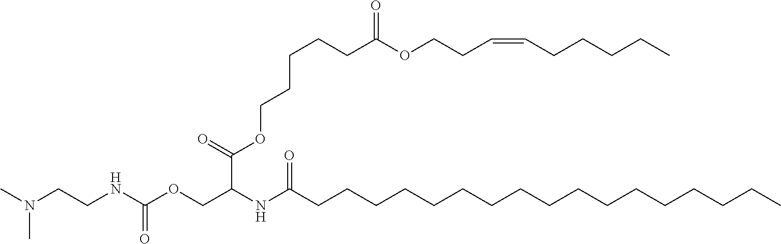 [SMILES: CCCCC/C=C\CCOC(=O)CCCCCOC(=O)C(COC(=O)NCCN(C)C)NC(=O)CCCCCCCCCCCCCCCCC] |
| 842. | 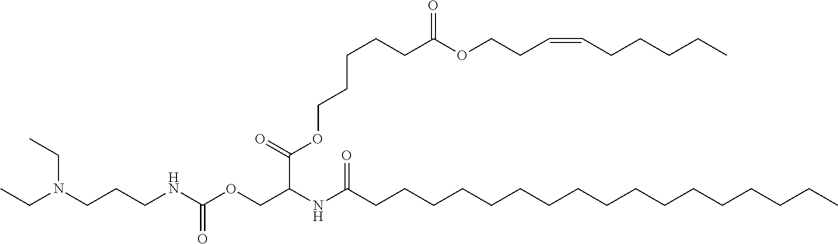 [SMILES: CCCCC/C=C\CCOC(=O)CCCCCOC(=O)C(COC(=O)NCCCN(CC)CC)NC(=O)CCCCCCCCCCCCCCCCC] |
| 843. | 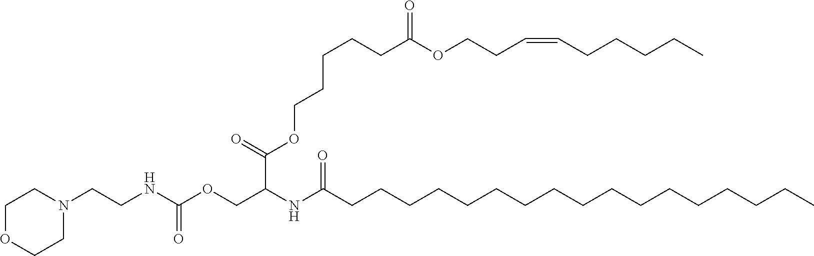 [SMILES: CCCCC/C=C\CCOC(=O)CCCCCOC(=O)C(COC(=O)NCCN1CCOCC1)NC(=O)CCCCCCCCCCCCCCCCC] |
| 844. | 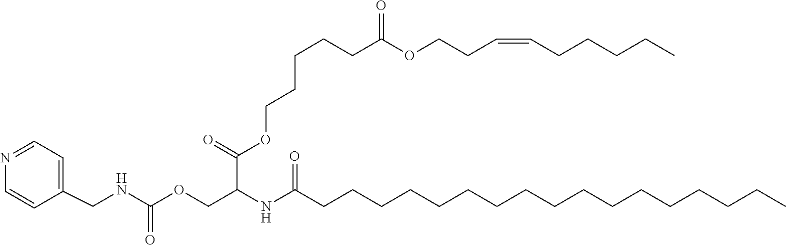 [SMILES: CCCCC/C=C\CCOC(=O)CCCCCOC(=O)C(COC(=O)NCc1ccncc1)NC(=O)CCCCCCCCCCCCCCCCC] |
| 845. | 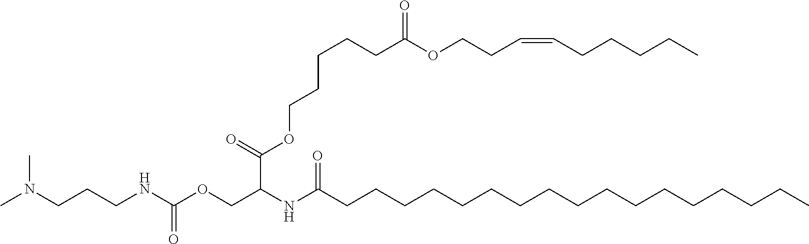 [SMILES: CCCCC/C=C\CCOC(=O)CCCCCOC(=O)C(COC(=O)NCCCN(C)C)NC(=O)CCCCCCCCCCCCCCCCC] |
| 846. | 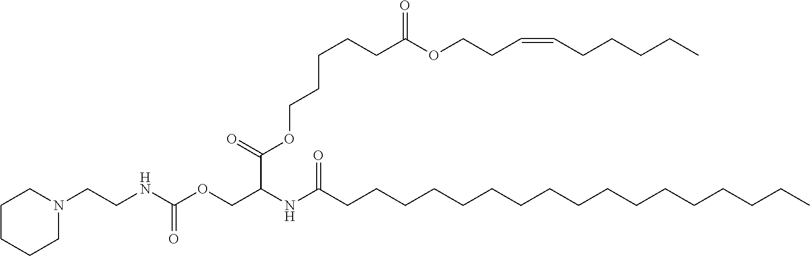 [SMILES: CCCCC/C=C\CCOC(=O)CCCCCOC(=O)C(COC(=O)NCCN1CCCCC1)NC(=O)CCCCCCCCCCCCCCCCC] |
| 847. | 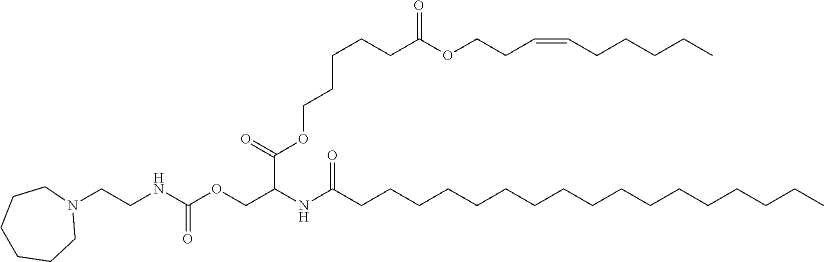 [SMILES: CCCCC/C=C\CCOC(=O)CCCCCOC(=O)C(COC(=O)NCCN1CCCCCC1)NC(=O)CCCCCCCCCCCCCCCCC] |
| 848. | 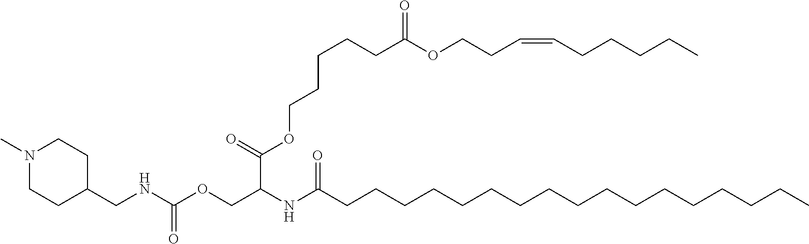 [SMILES: CCCCC/C=C\CCOC(=O)CCCCCOC(=O)C(COC(=O)NCC1CCN(C)CC1)NC(=O)CCCCCCCCCCCCCCCCC] |
| 849. | 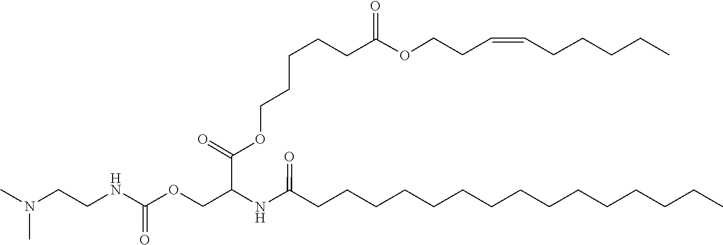 [SMILES: CCCCC/C=C\CCOC(=O)CCCCCOC(=O)C(COC(=O)NCCN(C)C)NC(=O)CCCCCCCCCCCCCCC] |
| 850. | 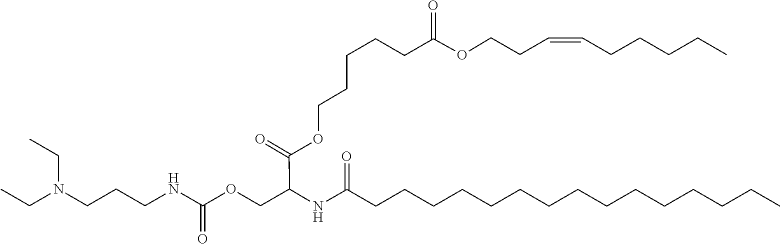 [SMILES: CCCCC/C=C\CCOC(=O)CCCCCOC(=O)C(COC(=O)NCCCN(CC)CC)NC(=O)CCCCCCCCCCCCCCC] |
| 851. | 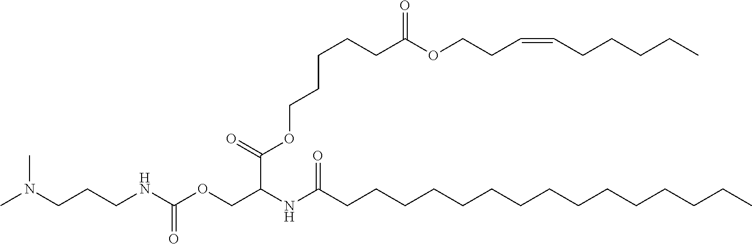 [SMILES: CCCCC/C=C\CCOC(=O)CCCCCOC(=O)C(COC(=O)NCCCN(C)C)NC(=O)CCCCCCCCCCCCCCC] |
| 852. | 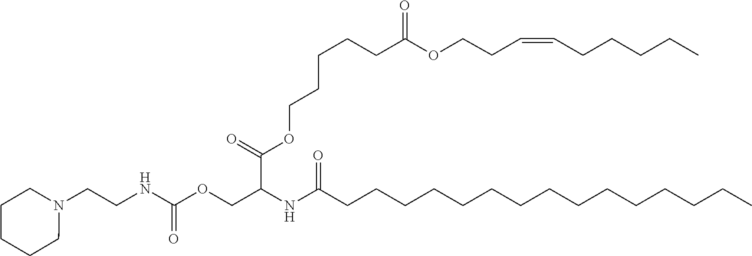 [SMILES: CCCCC/C=C\CCOC(=O)CCCCCOC(=O)C(COC(=O)NCCN1CCCCC1)NC(=O)CCCCCCCCCCCCCCC] |
| 853. | 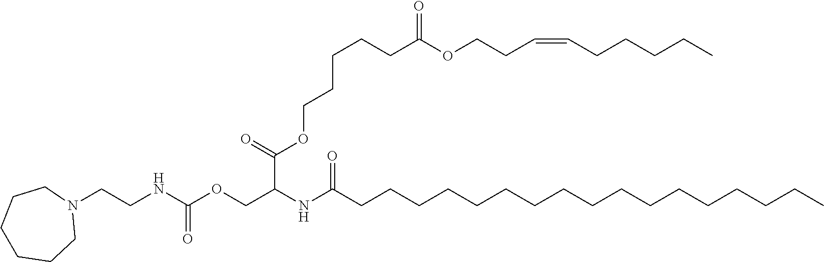 [SMILES: CCCCC/C=C\CCOC(=O)CCCCCOC(=O)C(COC(=O)NCCN1CCCCCC1)NC(=O)CCCCCCCCCCCCCCCCC] |
| 854. | 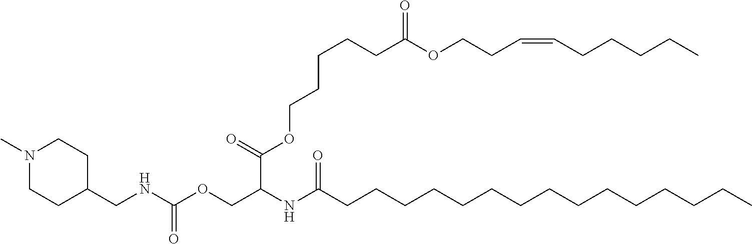 [SMILES: CCCCC/C=C\CCOC(=O)CCCCCOC(=O)C(COC(=O)NCC1CCN(C)CC1)NC(=O)CCCCCCCCCCCCCCC] |
| 855. | 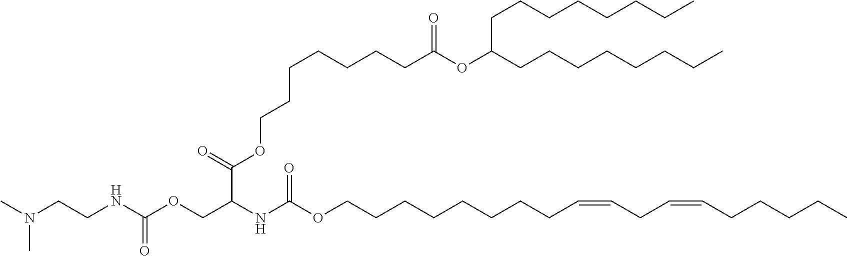 [SMILES: CCCCC/C=C\C/C=C\CCCCCCCCOC(=O)NC(COC(=O)NCCN(C)C)C(=O)OCCCCCCCC(=O)OC(CCCCCCCC)CCCCCCCC] |
| 856. | 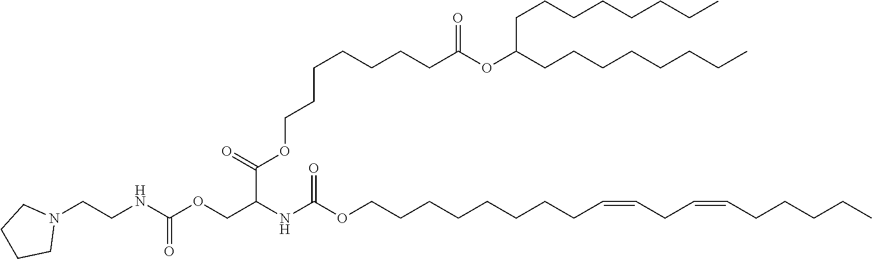 [SMILES: CCCCC/C=C\C/C=C\CCCCCCCCOC(=O)NC(COC(=O)NCCN1CCCC1)C(=O)OCCCCCCCC(=O)OC(CCCCCCCC)CCCCCCCC] |
| 857. | 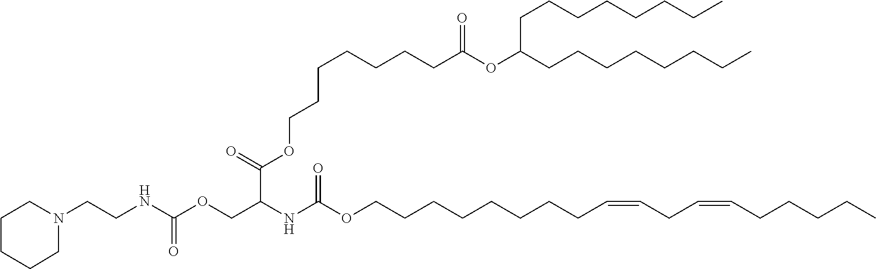 [SMILES: CCCCC/C=C\C/C=C\CCCCCCCCOC(=O)NC(COC(=O)NCCN1CCCCC1)C(=O)OCCCCCCCC(=O)OC(CCCCCCCC)CCCCCCCC] |
| 858. | 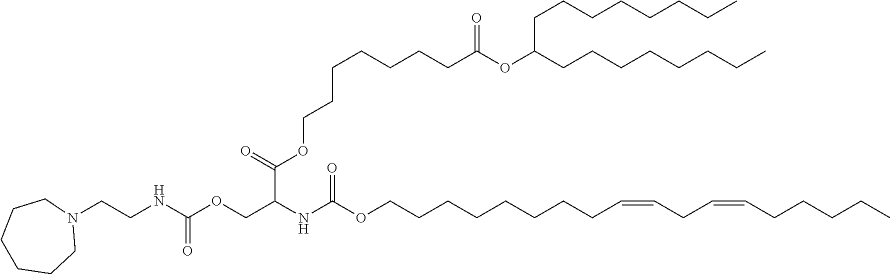 [SMILES: CCCCC/C=C\C/C=C\CCCCCCCCOC(=O)NC(COC(=O)NCCN1CCCCCC1)C(=O)OCCCCCCCC(=O)OC(CCCCCCCC)CCCCCCCC] |
| 859. | 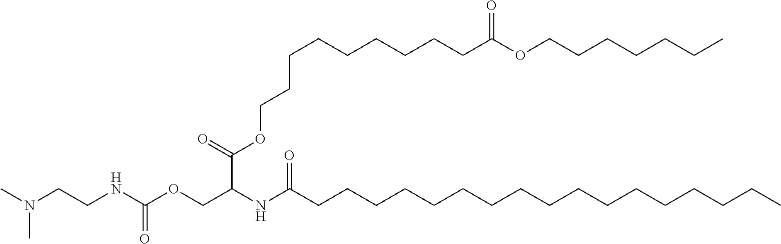 [SMILES: CCCCCCCCCCCCCCCCCC(=O)NC(COC(=O)NCCN(C)C)C(=O)OCCCCCCCCCC(=O)OCCCCCCC] |
| 860. | 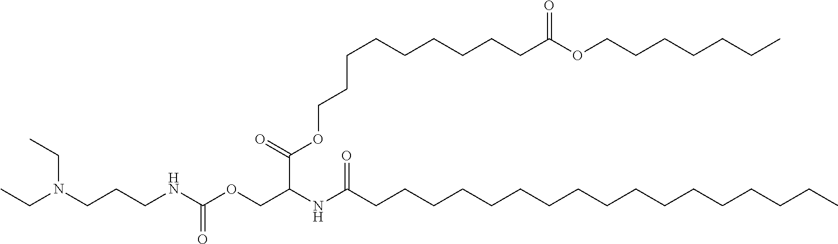 [SMILES: CCCCCCCCCCCCCCCCCC(=O)NC(COC(=O)NCCCN(CC)CC)C(=O)OCCCCCCCCCC(=O)OCCCCCCC] |
| 861. | 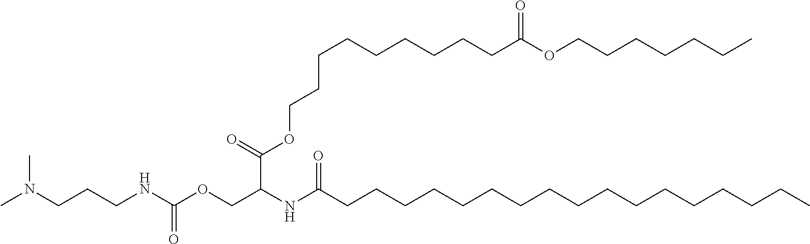 [SMILES: CCCCCCCCCCCCCCCCCC(=O)NC(COC(=O)NCCCN(C)C)C(=O)OCCCCCCCCCC(=O)OCCCCCCC] |
| 862. | 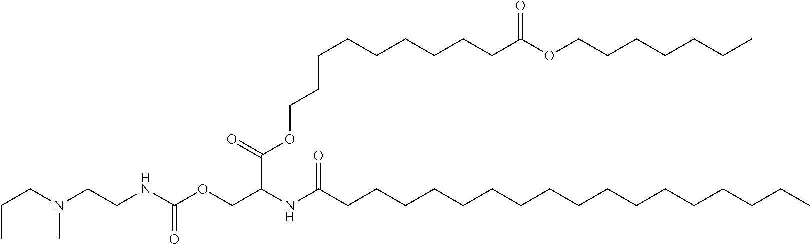 [SMILES: CCCCCCCCCCCCCCCCCC(=O)NC(COC(=O)NCCN(C)CCC)C(=O)OCCCCCCCCCC(=O)OCCCCCCC] |
| 863. | 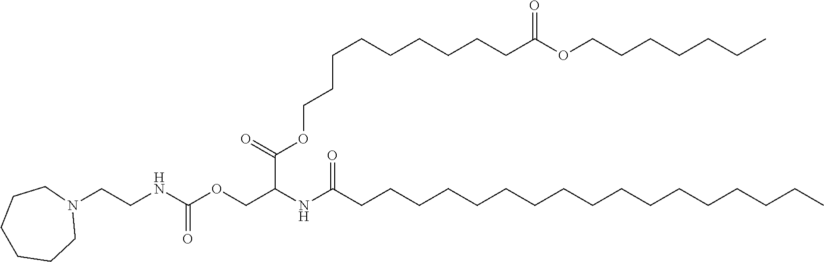 [SMILES: CCCCCCCCCCCCCCCCCC(=O)NC(COC(=O)NCCN1CCCCCC1)C(=O)OCCCCCCCCCC(=O)OCCCCCCC] |
| 864. | 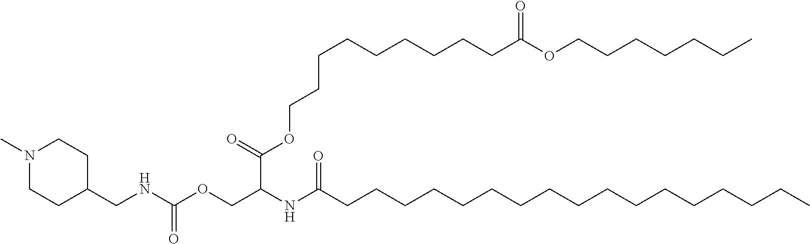 [SMILES: CCCCCCCCCCCCCCCCCC(=O)NC(COC(=O)NCC1CCN(C)CC1)C(=O)OCCCCCCCCCC(=O)OCCCCCCC] |
| 865. | 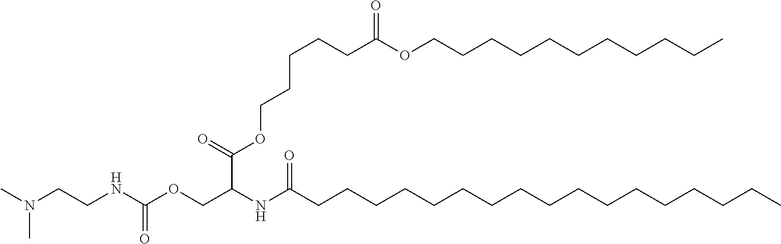 [SMILES: CCCCCCCCCCCCCCCCCC(=O)NC(COC(=O)NCCN(C)C)C(=O)OCCCCCC(=O)OCCCCCCCCCCC] |
| 866. | 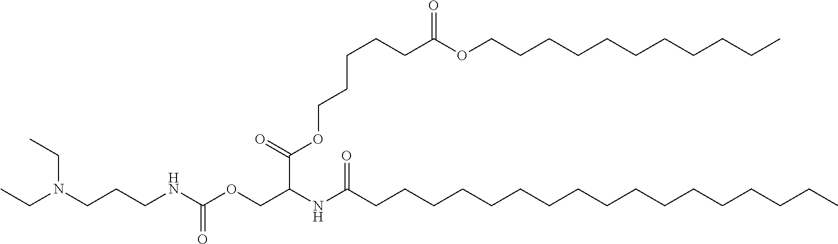 [SMILES: CCCCCCCCCCCCCCCCCC(=O)NC(COC(=O)NCCCN(CC)CC)C(=O)OCCCCCC(=O)OCCCCCCCCCCC] |
| 867. | 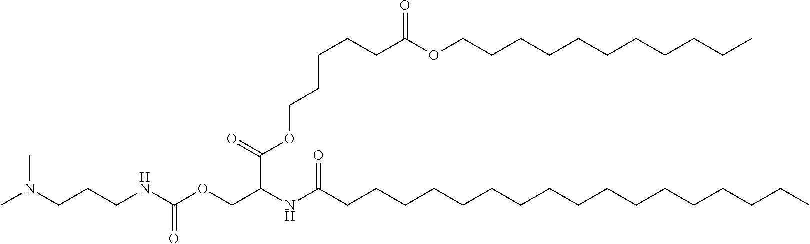 [SMILES: CCCCCCCCCCCCCCCCCC(=O)NC(COC(=O)NCCCN(C)C)C(=O)OCCCCCC(=O)OCCCCCCCCCCC] |
| 868. | 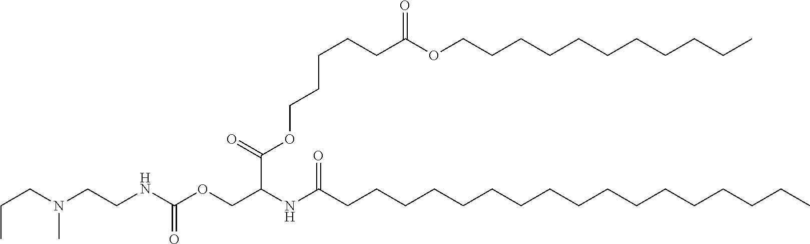 [SMILES: CCCCCCCCCCCCCCCCCC(=O)NC(COC(=O)NCCN(C)CCC)C(=O)OCCCCCC(=O)OCCCCCCCCCCC] |
| 869. | 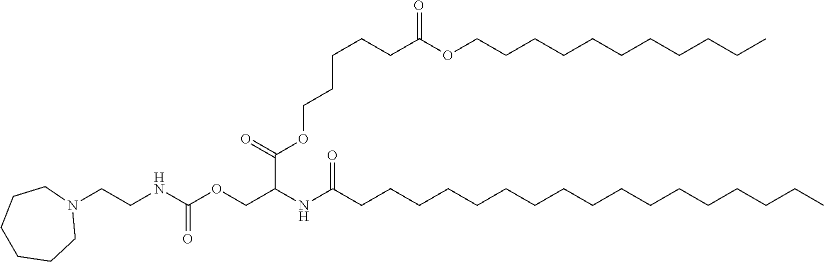 [SMILES: CCCCCCCCCCCCCCCCCC(=O)NC(COC(=O)NCCN1CCCCCC1)C(=O)OCCCCCC(=O)OCCCCCCCCCCC] |
| 870. | 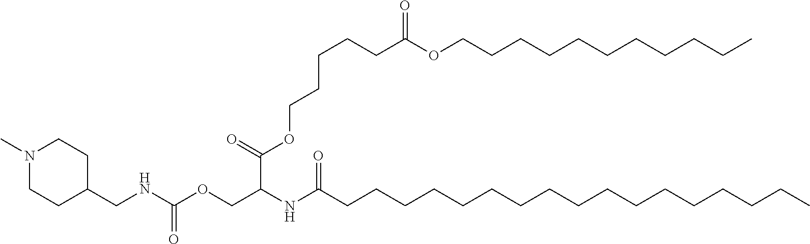 [SMILES: CCCCCCCCCCCCCCCCCC(=O)NC(COC(=O)NCC1CCN(C)CC1)C(=O)OCCCCCC(=O)OCCCCCCCCCCC] |
| 871. | 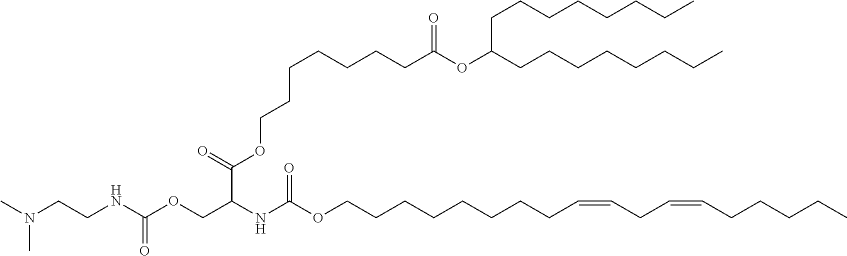 [SMILES: CCCCC/C=C\C/C=C\CCCCCCCCOC(=O)NC(COC(=O)NCCN(C)C)C(=O)OCCCCCCCC(=O)OC(CCCCCCCC)CCCCCCCC] |
| 872. | 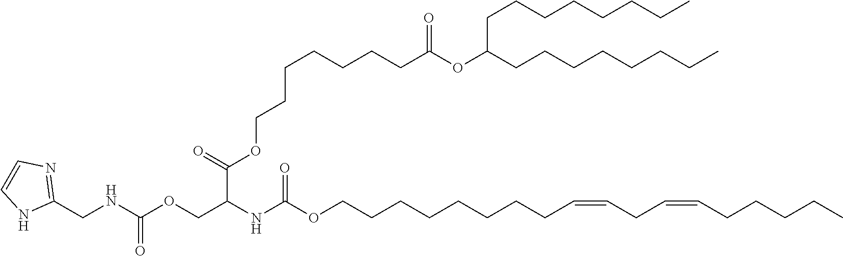 [SMILES: CCCCC/C=C\C/C=C\CCCCCCCCOC(=O)NC(COC(=O)NCc1ncc[nH]1)C(=O)OCCCCCCCC(=O)OC(CCCCCCCC)CCCCCCCC] |
| 873. | 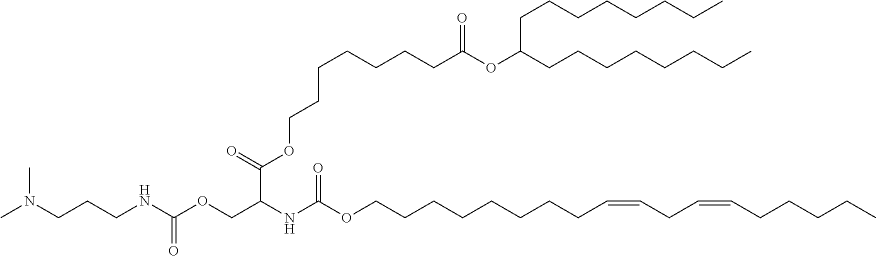 [SMILES: CCCCC/C=C\C/C=C\CCCCCCCCOC(=O)NC(COC(=O)NCCCN(C)C)C(=O)OCCCCCCCC(=O)OC(CCCCCCCC)CCCCCCCC] |
| 874. | 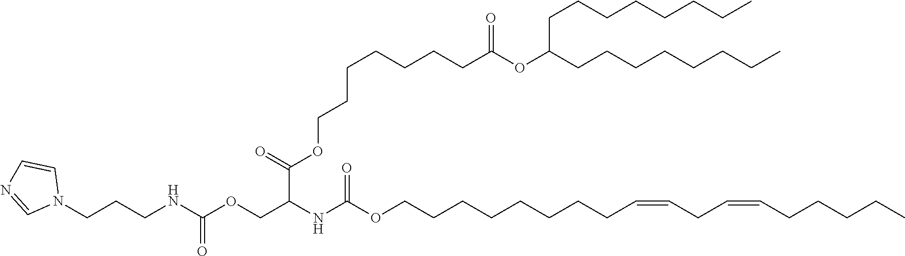 [SMILES: CCCCC/C=C\C/C=C\CCCCCCCCOC(=O)NC(COC(=O)NCCCn1ccnc1)C(=O)OCCCCCCCC(=O)OC(CCCCCCCC)CCCCCCCC] |
| 875. | 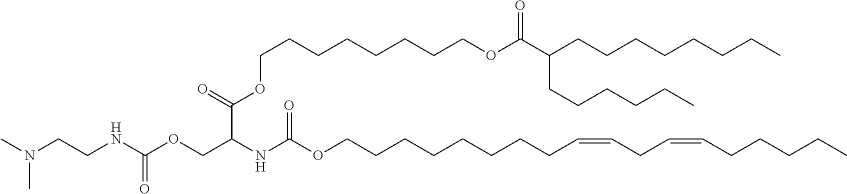 [SMILES: CCCCC/C=C\C/C=C\CCCCCCCCOC(=O)NC(COC(=O)NCCN(C)C)C(=O)OCCCCCCCCOC(=O)C(CCCCCC)CCCCCCCC] |
| 876. | 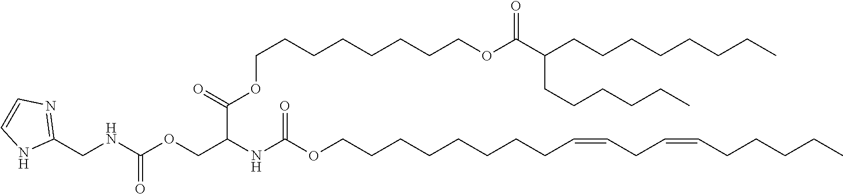 [SMILES: CCCCC/C=C\C/C=C\CCCCCCCCOC(=O)NC(COC(=O)NCc1ncc[nH]1)C(=O)OCCCCCCCCOC(=O)C(CCCCCC)CCCCCCCC] |
| 877. | 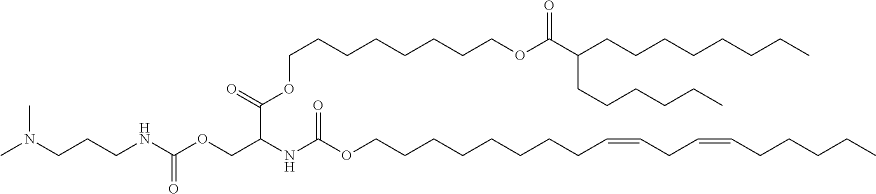 [SMILES: CCCCC/C=C\C/C=C\CCCCCCCCOC(=O)NC(COC(=O)NCCCN(C)C)C(=O)OCCCCCCCCOC(=O)C(CCCCCC)CCCCCCCC] |
| 878. | 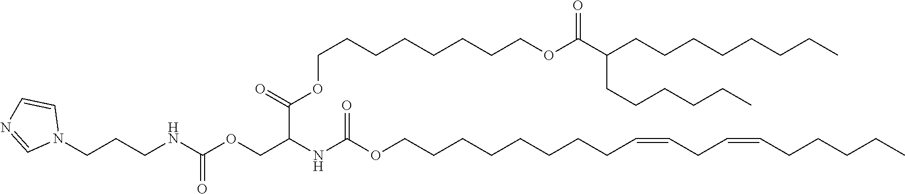 [SMILES: CCCCC/C=C\C/C=C\CCCCCCCCOC(=O)NC(COC(=O)NCCCn1ccnc1)C(=O)OCCCCCCCCOC(=O)C(CCCCCC)CCCCCCCC] |
| 879. | 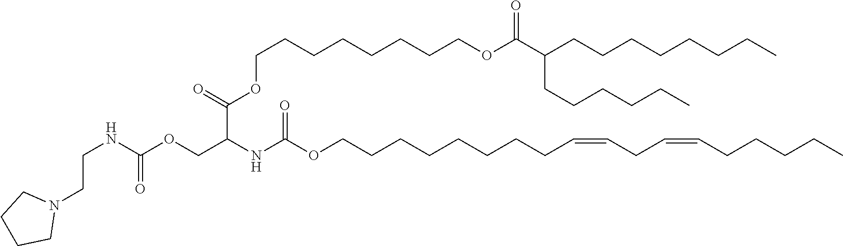 [SMILES: CCCCC/C=C\C/C=C\CCCCCCCCOC(=O)NC(COC(=O)NCCN1CCCC1)C(=O)OCCCCCCCCOC(=O)C(CCCCCC)CCCCCCCC] |
| 880. | 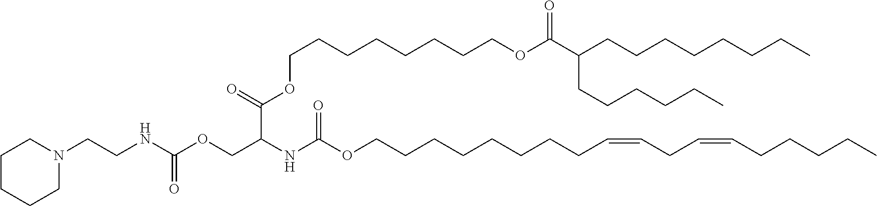 [SMILES: CCCCC/C=C\C/C=C\CCCCCCCCOC(=O)NC(COC(=O)NCCN1CCCCC1)C(=O)OCCCCCCCCOC(=O)C(CCCCCC)CCCCCCCC] |
| 881. | 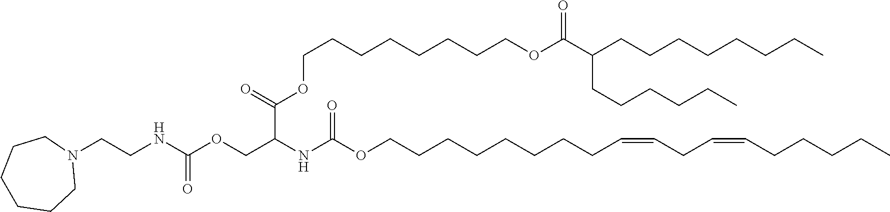 [SMILES: CCCCC/C=C\C/C=C\CCCCCCCCOC(=O)NC(COC(=O)NCCN1CCCCCC1)C(=O)OCCCCCCCCOC(=O)C(CCCCCC)CCCCCCCC] |
| 882. | 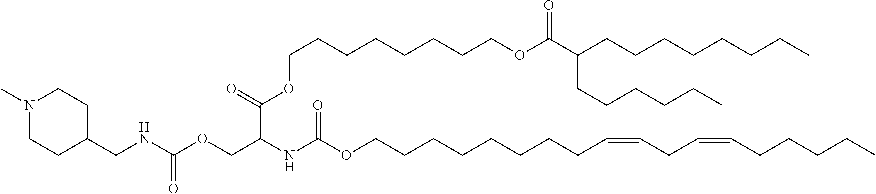 [SMILES: CCCCC/C=C\C/C=C\CCCCCCCCOC(=O)NC(COC(=O)NCC1CCN(C)CC1)C(=O)OCCCCCCCCOC(=O)C(CCCCCC)CCCCCCCC] |
| 883. | 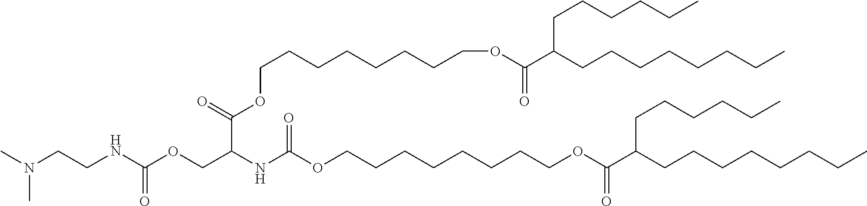 |
| 884. | 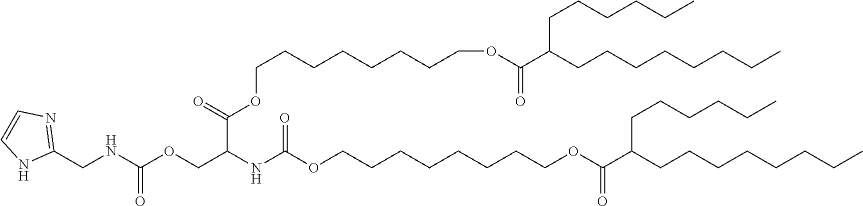 |
| 885. | 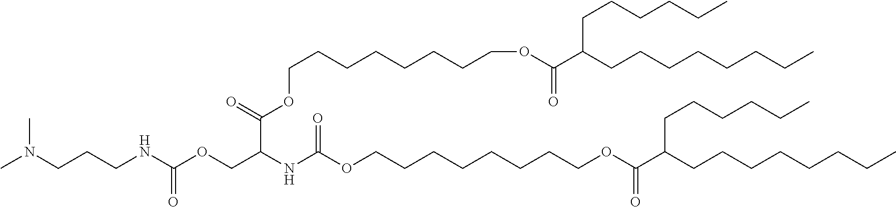 |
| 886. | 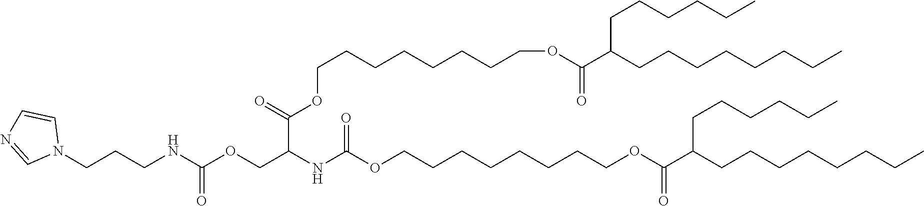 |
| 887. | 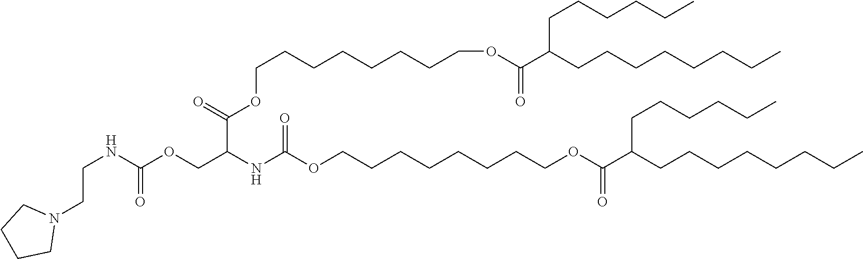 |
| 888. | 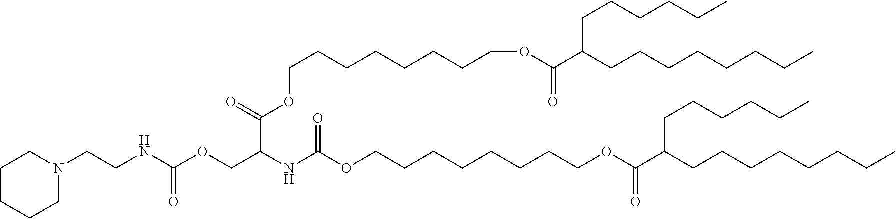 |
| 889. | 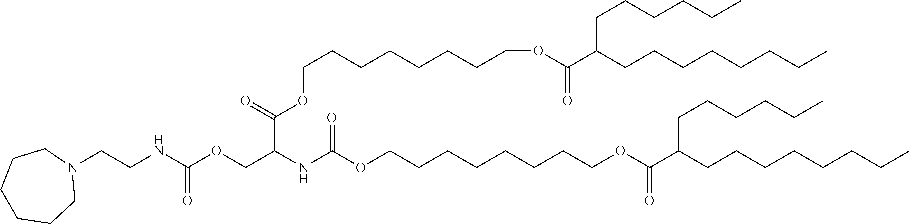 |
| 890. | 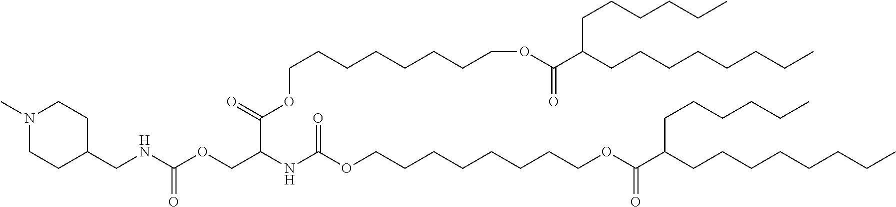 |
| 891. | 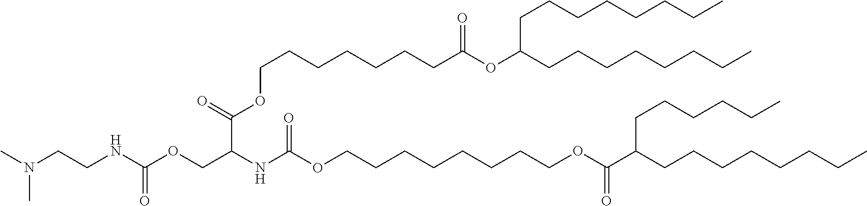 |
| 892. | 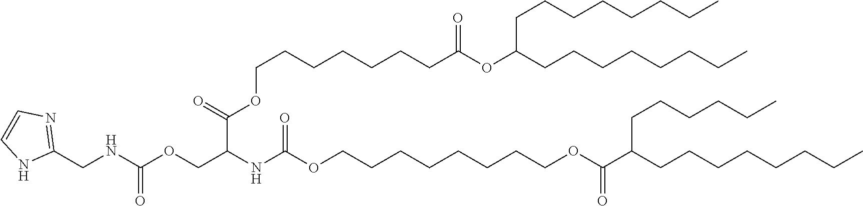 |
| 893. | 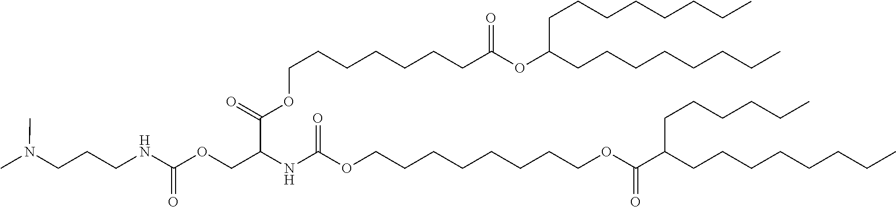 |
| 894. | 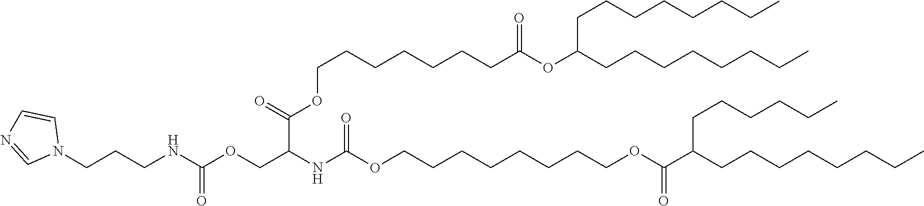 |
| 895. | 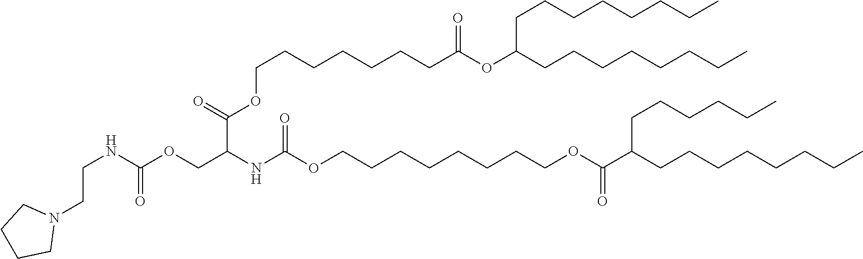 |
| 896. | 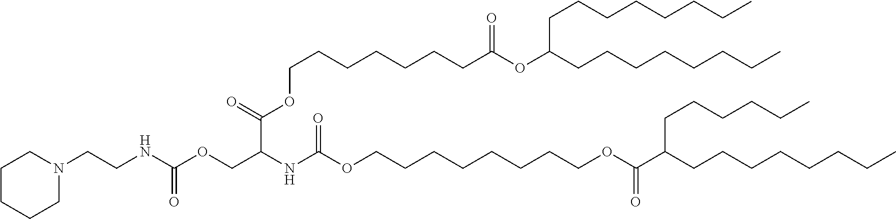 |
| 897. | 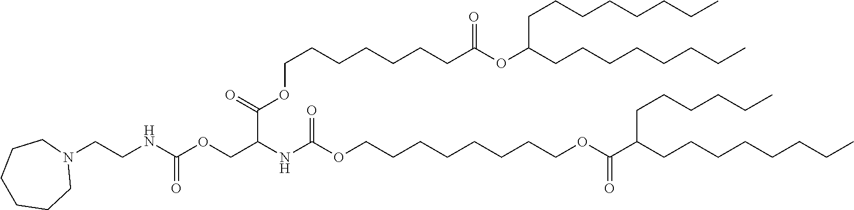 |
| 898. | 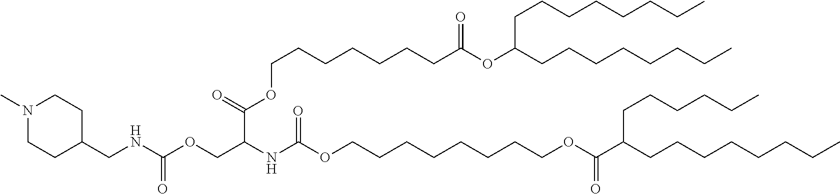 |
| 899. | 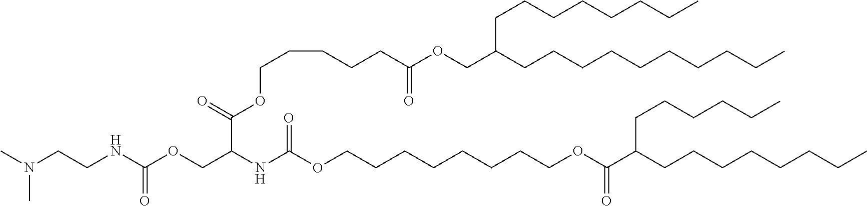 |
| 900. | 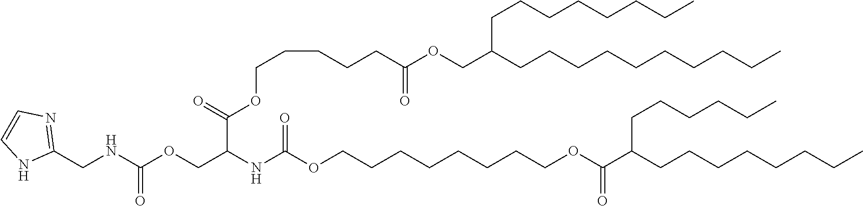 |
| 901. | 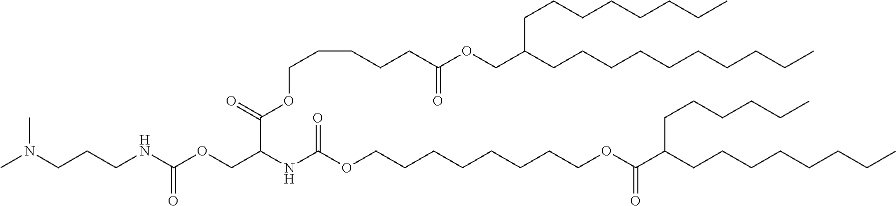 |
| 902. | 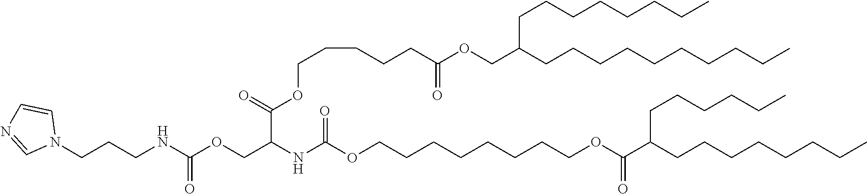 |
| 903. | 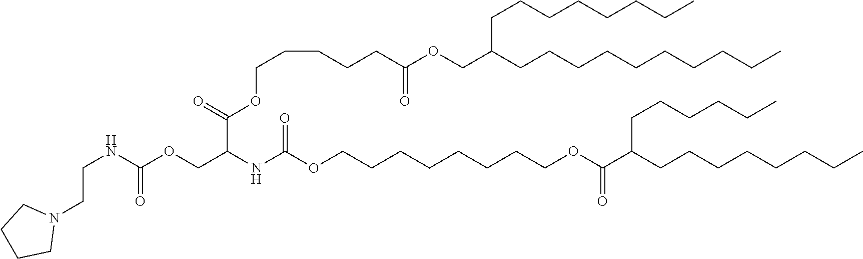 |
| 904. | 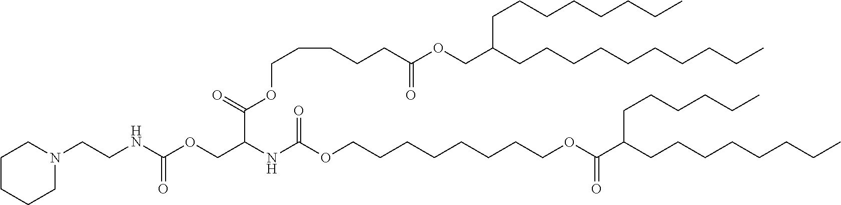 |
| 905. | 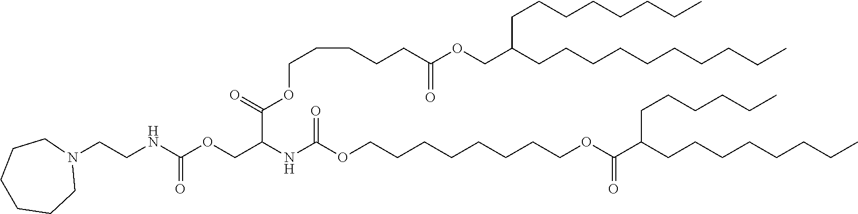 |
| 906. | 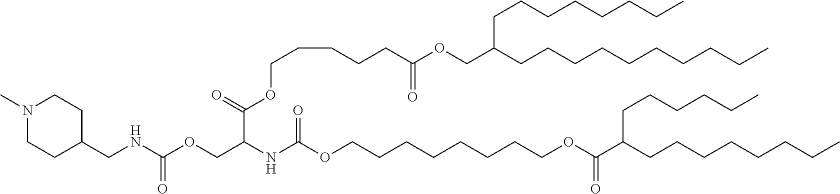 |
| 907. | 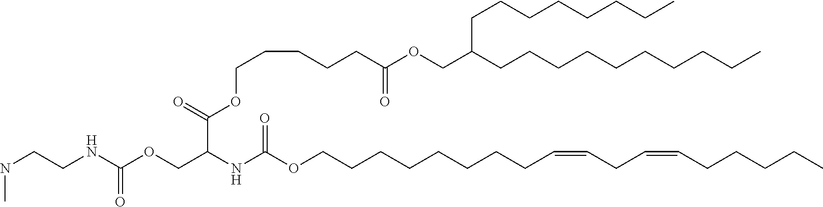 [SMILES: CCCCC/C=C\C/C=C\CCCCCCCCOC(=O)NC(COC(=O)NCCNC)C(=O)OCCCCCC(=O)OCC(CCCCCCCC)CCCCCCCCCC] |
| 908. | 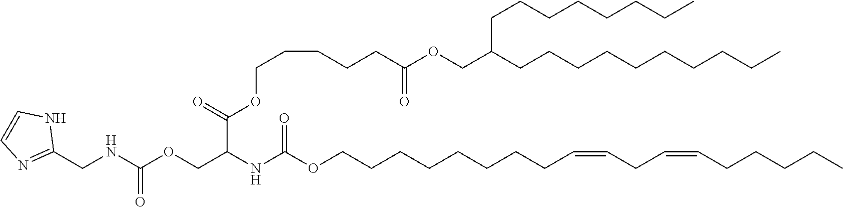 [SMILES: CCCCC/C=C\C/C=C\CCCCCCCCOC(=O)NC(COC(=O)NCc1ncc[nH]1)C(=O)OCCCCCC(=O)OCC(CCCCCCCC)CCCCCCCCCC] |
| 909. | 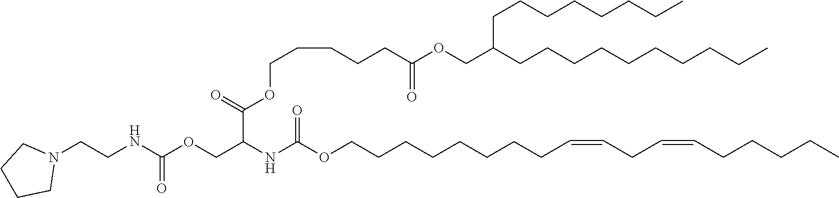 |
| 910. | 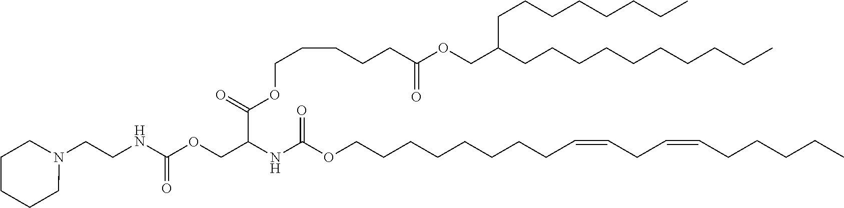 |
| 911. | 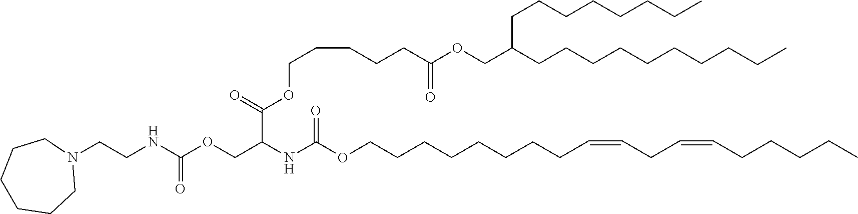 |
| 912. | 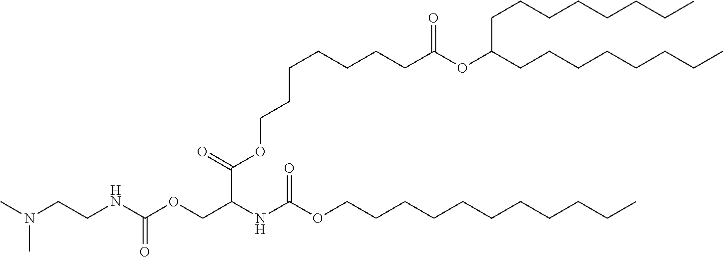 [SMILES: CCCCCCCCCCCOC(=O)NC(COC(=O)NCCN(C)C)C(=O)OCCCCCCCC(=O)OC(CCCCCCCC)CCCCCCCC] |
| 913. | 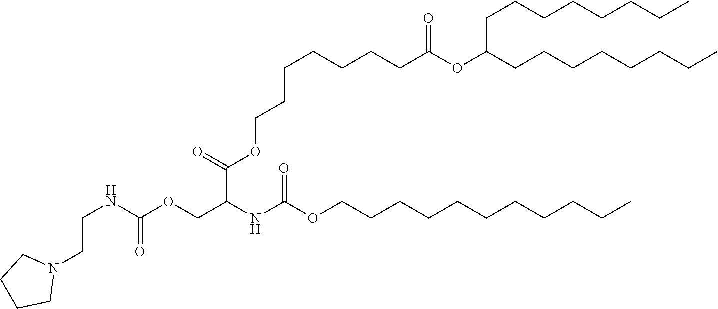 [SMILES: CCCCCCCCCCCOC(=O)NC(COC(=O)NCCN1CCCC1)C(=O)OCCCCCCCC(=O)OC(CCCCCCCC)CCCCCCCC] |
| 914. | 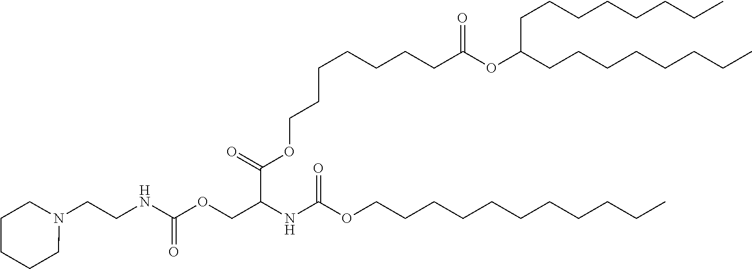 [SMILES: CCCCCCCCCCCOC(=O)NC(COC(=O)NCCN1CCCCC1)C(=O)OCCCCCCCC(=O)OC(CCCCCCCC)CCCCCCCC] |
| 915. | 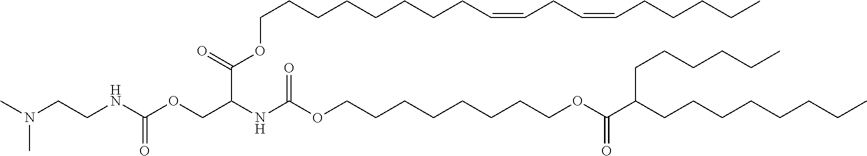 [SMILES: CCCCC/C=C\C/C=C\CCCCCCCCOC(=O)C(COC(=O)NCCN(C)C)NC(=O)OCCCCCCCCOC(=O)C(CCCCCC)CCCCCCCC] |
| 916. | 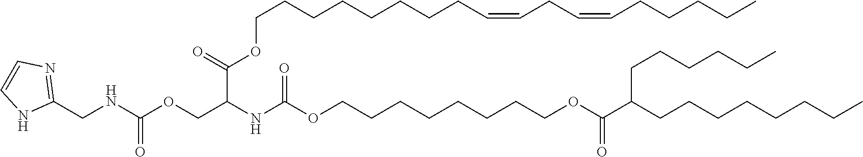 [SMILES: CCCCC/C=C\C/C=C\CCCCCCCCOC(=O)C(COC(=O)NCc1ncc[nH]1)NC(=O)OCCCCCCCCOC(=O)C(CCCCCC)CCCCCCCC] |
| 917. | 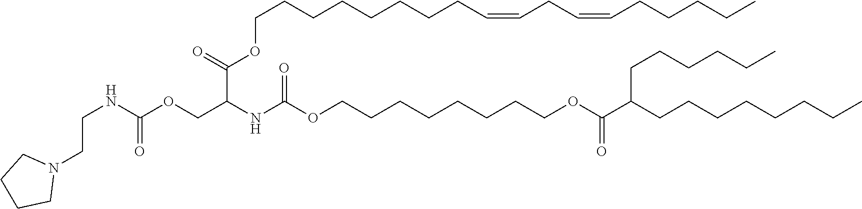 [SMILES: CCCCC/C=C\C/C=C\CCCCCCCCOC(=O)C(COC(=O)NCCN1CCCC1)NC(=O)OCCCCCCCCOC(=O)C(CCCCCC)CCCCCCCC] |
| 918. | 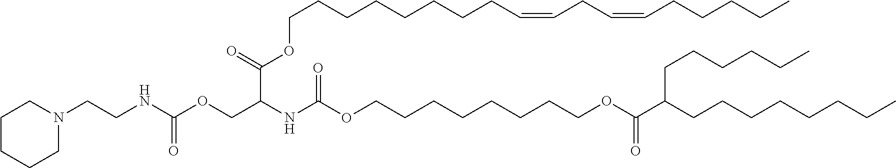 [SMILES: CCCCC/C=C\C/C=C\CCCCCCCCOC(=O)C(COC(=O)NCCN1CCCCC1)NC(=O)OCCCCCCCCOC(=O)C(CCCCCC)CCCCCCCC] |
| 919. | 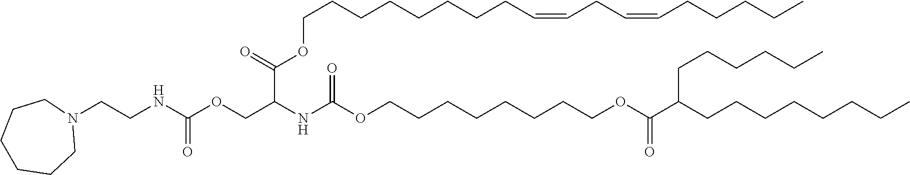 [SMILES: CCCCC/C=C\C/C=C\CCCCCCCCOC(=O)C(COC(=O)NCCN1CCCCCC1)NC(=O)OCCCCCCCCOC(=O)C(CCCCCC)CCCCCCCC] |
| 920. | 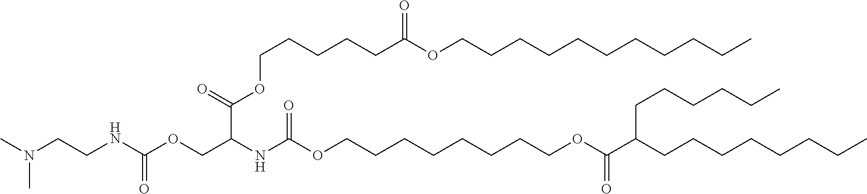 |
| 921. | 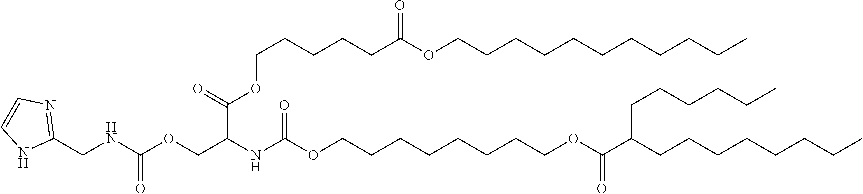 |
| 922. | 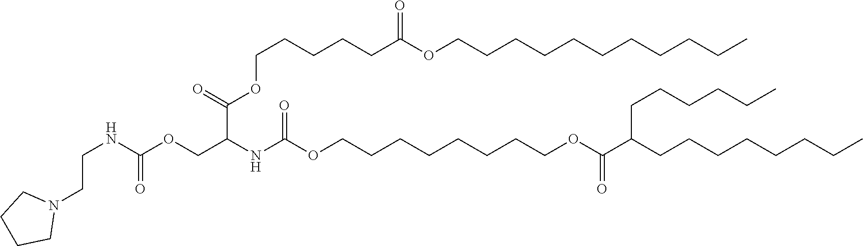 [SMILES: CCCCCCCCCCCOC(=O)CCCCCOC(=O)C(COC(=O)NCCN1CCCC1)NC(=O)OCCCCCCCCOC(=O)C(CCCCCC)CCCCCCCC] |
| 923. | 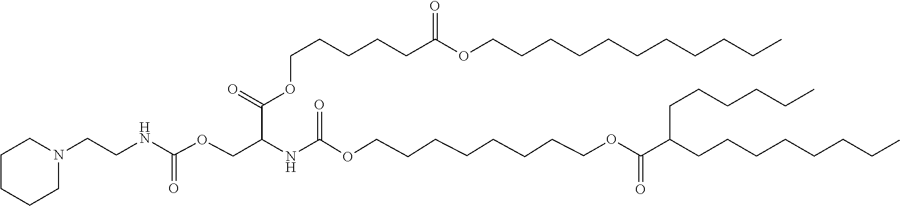 [SMILES: CCCCCCCCCCCOC(=O)CCCCCOC(=O)C(COC(=O)NCCN1CCCCC1)NC(=O)OCCCCCCCCOC(=O)C(CCCCCC)CCCCCCCC] |
| 924. | 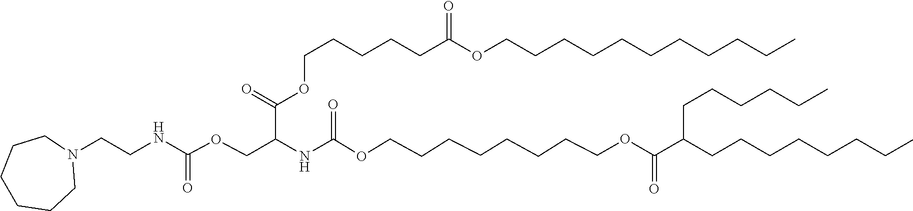 [SMILES: CCCCCCCCCCCOC(=O)CCCCCOC(=O)C(COC(=O)NCCN1CCCCCC1)NC(=O)OCCCCCCCCOC(=O)C(CCCCCC)CCCCCCCC] |
| 925. | 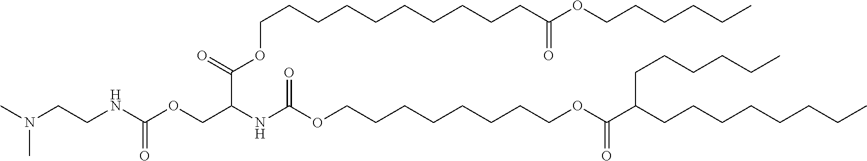 [SMILES: CCCCCCCCC(CCCCCC)C(=O)OCCCCCCCCOC(=O)NC(COC(=O)NCCN(C)C)C(=O)OCCCCCCCCCCC(=O)OCCCCCC] |
| 926. | 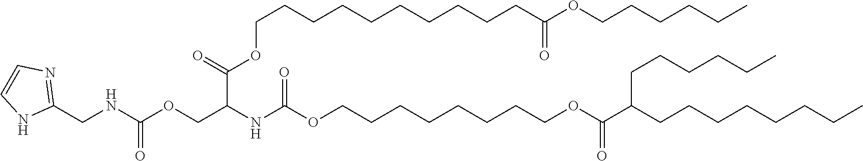 [SMILES: CCCCCCCCC(CCCCCC)C(=O)OCCCCCCCCOC(=O)NC(COC(=O)NCc1ncc[nH]1)C(=O)OCCCCCCCCCCC(=O)OCCCCCC] |
| 927. | 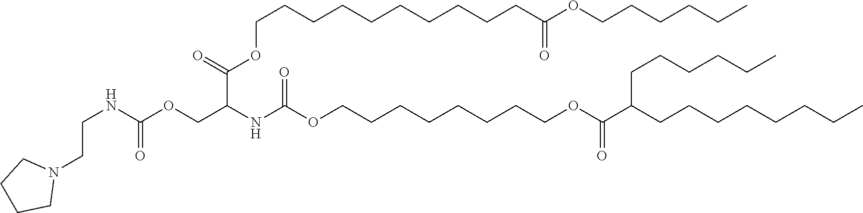 [SMILES: CCCCCCCCC(CCCCCC)C(=O)OCCCCCCCCOC(=O)NC(COC(=O)NCCN1CCCC1)C(=O)OCCCCCCCCCCC(=O)OCCCCCC] |
| 928. | 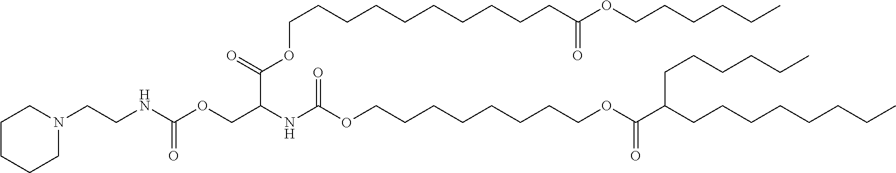 [SMILES: CCCCCCCCC(CCCCCC)C(=O)OCCCCCCCCOC(=O)NC(COC(=O)NCCN1CCCCC1)C(=O)OCCCCCCCCCCC(=O)OCCCCCC] |
| 929. | 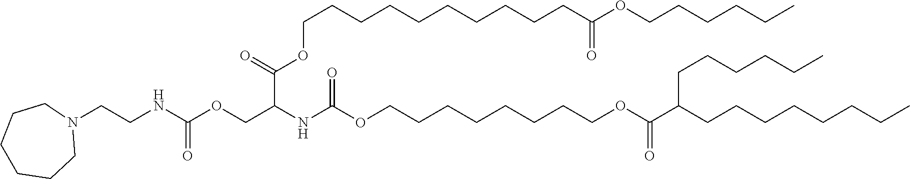 [SMILES: CCCCCCCCC(CCCCCC)C(=O)OCCCCCCCCOC(=O)NC(COC(=O)NCCN1CCCCCC1)C(=O)OCCCCCCCCCCC(=O)OCCCCCC] |
| 930. | 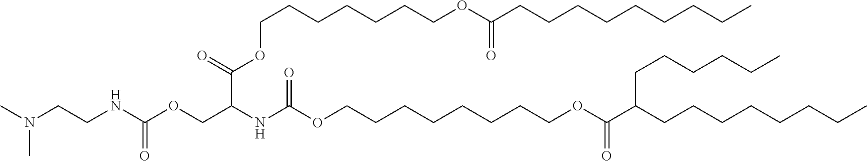 [SMILES: CCCCCCCCCC(=O)OCCCCCCCOC(=O)C(COC(=O)NCCN(C)C)NC(=O)OCCCCCCCCOC(=O)C(CCCCCC)CCCCCCCC] |
| 931. | 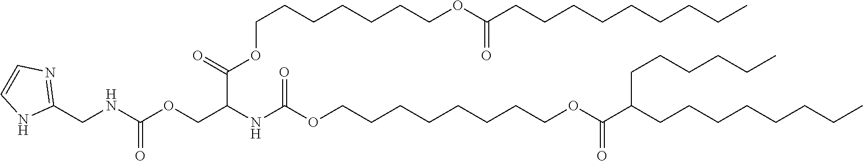 [SMILES: CCCCCCCCCC(=O)OCCCCCCCOC(=O)C(COC(=O)NCc1ncc[nH]1)NC(=O)OCCCCCCCCOC(=O)C(CCCCCC)CCCCCCCC] |
| 932. | 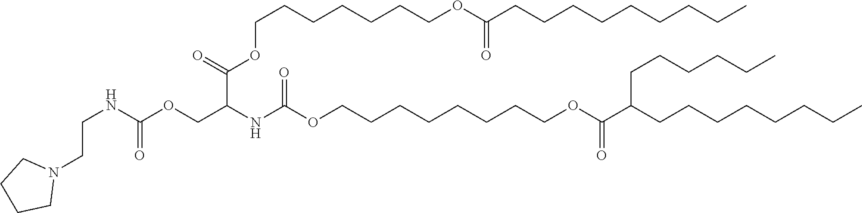 [SMILES: CCCCCCCCCC(=O)OCCCCCCCOC(=O)C(COC(=O)NCCN1CCCC1)NC(=O)OCCCCCCCCOC(=O)C(CCCCCC)CCCCCCCC] |
| 933. | 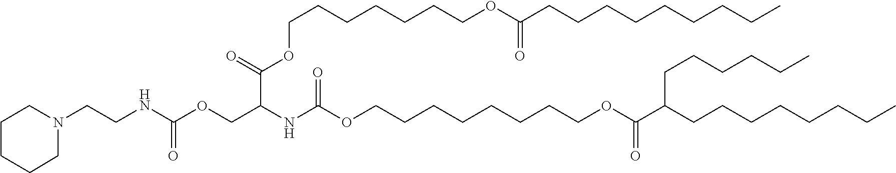 [SMILES: CCCCCCCCCC(=O)OCCCCCCCOC(=O)C(COC(=O)NCCN1CCCCC1)NC(=O)OCCCCCCCCOC(=O)C(CCCCCC)CCCCCCCC] |
| 934. | 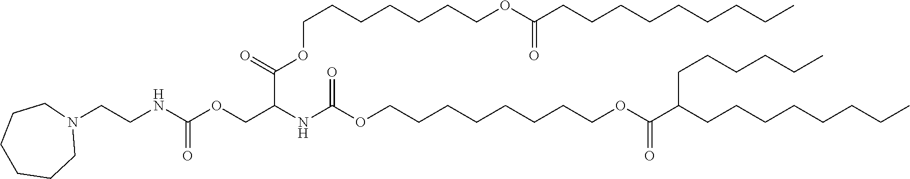 [SMILES: CCCCCCCCCC(=O)OCCCCCCCOC(=O)C(COC(=O)NCCN1CCCCCC1)NC(=O)OCCCCCCCCOC(=O)C(CCCCCC)CCCCCCCC] |
| 935. | 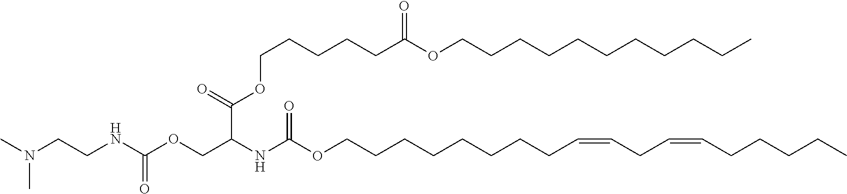 |
| 936. | 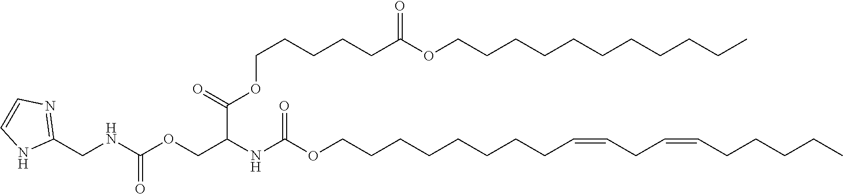 |
| 937. | 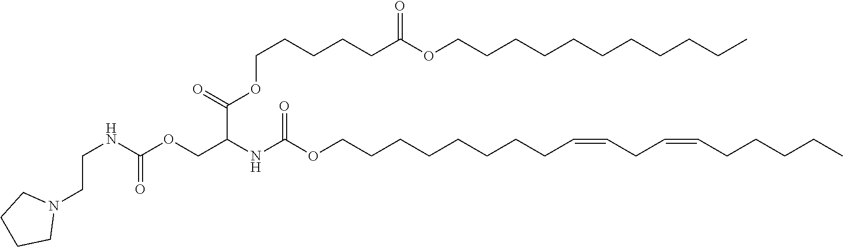 |
| 938. | 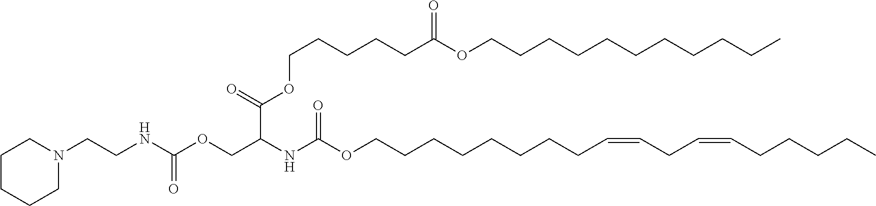 |
| 939. | 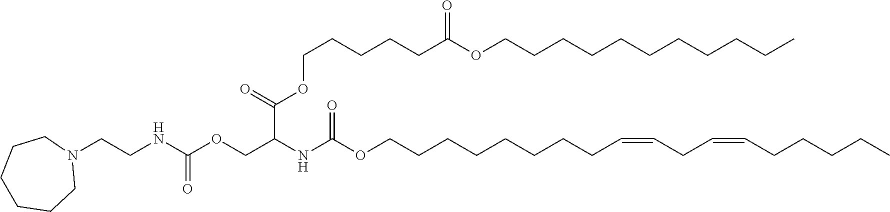 |
| 940. | 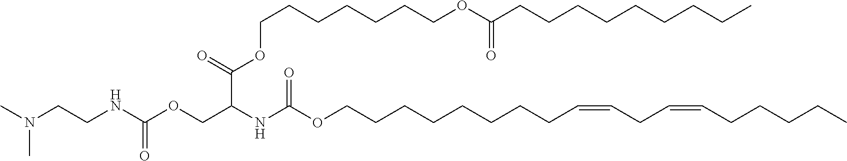 [SMILES: CCCCC/C=C\C/C=C\CCCCCCCCOC(=O)NC(COC(=O)NCCN(C)C)C(=O)OCCCCCCCOC(=O)CCCCCCCCC] |
| 941. | 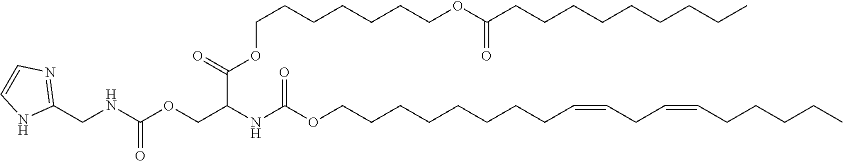 [SMILES: CCCCC/C=C\C/C=C\CCCCCCCCOC(=O)NC(COC(=O)NCc1ncc[nH]1)C(=O)OCCCCCCCOC(=O)CCCCCCCCC] |
| 942. | 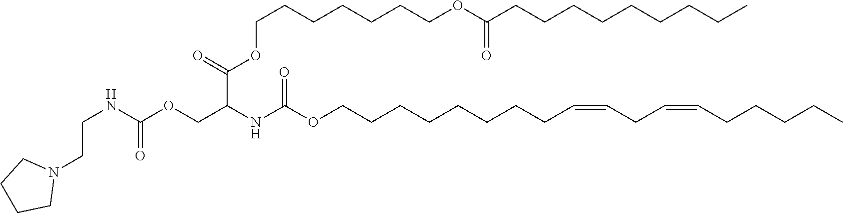 [SMILES: CCCCC/C=C\C/C=C\CCCCCCCCOC(=O)NC(COC(=O)NCCN1CCCC1)C(=O)OCCCCCCCOC(=O)CCCCCCCCC] |
| 943. | 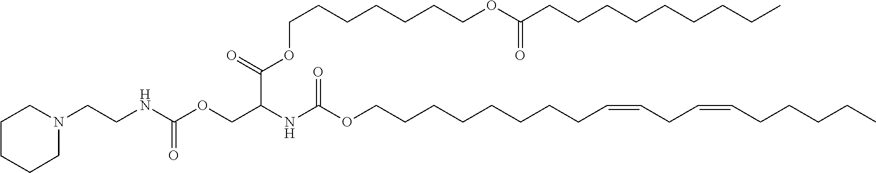 [SMILES: CCCCC/C=C\C/C=C\CCCCCCCCOC(=O)NC(COC(=O)NCCN1CCCCC1)C(=O)OCCCCCCCOC(=O)CCCCCCCCC] |
| 944. | 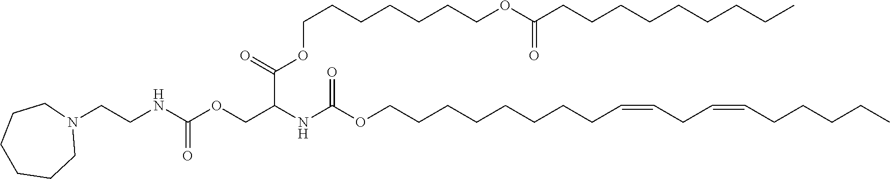 [SMILES: CCCCC/C=C\C/C=C\CCCCCCCCOC(=O)NC(COC(=O)NCCN1CCCCCC1)C(=O)OCCCCCCCOC(=O)CCCCCCCCC] |
| 945. | 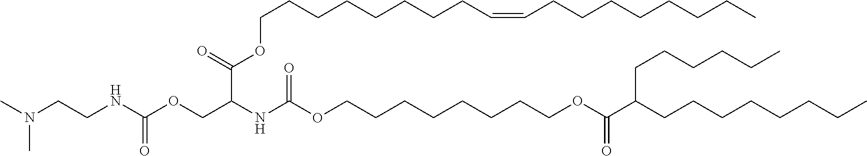 [SMILES: CCCCCCCC/C=C\CCCCCCCCOC(=O)C(COC(=O)NCCN(C)C)NC(=O)OCCCCCCCCOC(=O)C(CCCCCC)CCCCCCCC] |
| 946. | 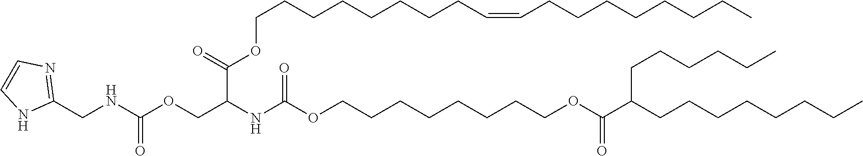 [SMILES: CCCCCCCC/C=C\CCCCCCCCOC(=O)C(COC(=O)NCc1ncc[nH]1)NC(=O)OCCCCCCCCOC(=O)C(CCCCCC)CCCCCCCC] |
| 947. | 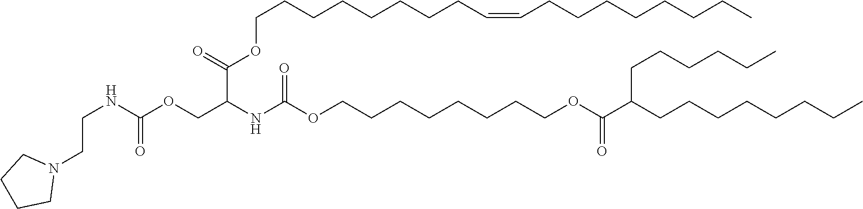 [SMILES: CCCCCCCC/C=C\CCCCCCCCOC(=O)C(COC(=O)NCCN1CCCC1)NC(=O)OCCCCCCCCOC(=O)C(CCCCCC)CCCCCCCC] |
| 948. | 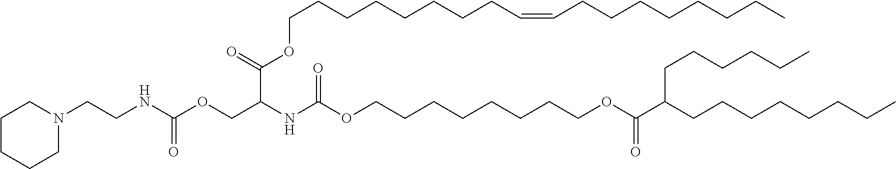 [SMILES: CCCCCCCC/C=C\CCCCCCCCOC(=O)C(COC(=O)NCCN1CCCCC1)NC(=O)OCCCCCCCCOC(=O)C(CCCCCC)CCCCCCCC] |
| 949. | 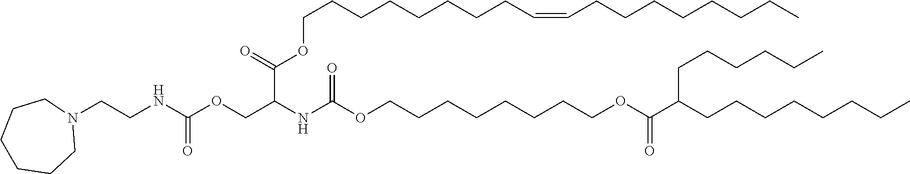 [SMILES: CCCCCCCC/C=C\CCCCCCCCOC(=O)C(COC(=O)NCCN1CCCCCC1)NC(=O)OCCCCCCCCOC(=O)C(CCCCCC)CCCCCCCC] |
| 950. | 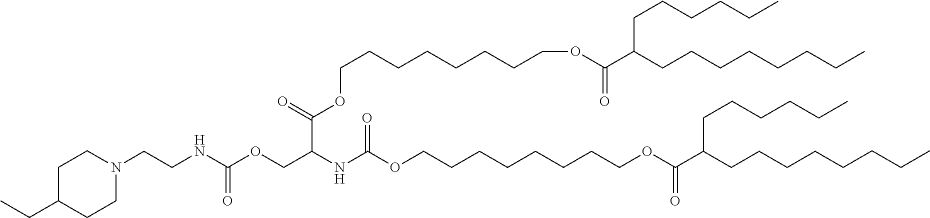 [SMILES: CCCCCCCCC(CCCCCC)C(=O)OCCCCCCCCOC(=O)NC(COC(=O)NCCN1CCC(CC)CC1)C(=O)OCCCCCCCCOC(=O)C(CCCCCC)CCCCCCCC] |
| 951. | 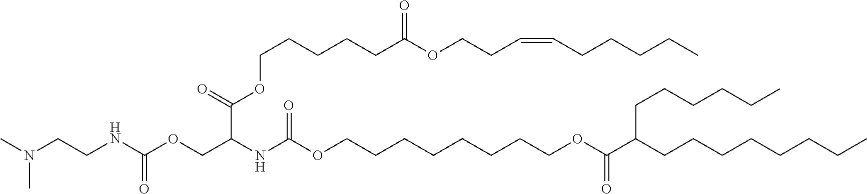 |
| 952. | 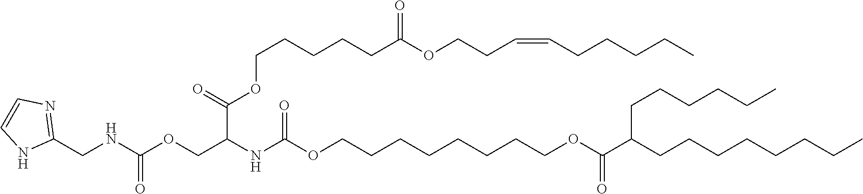 [SMILES: CCCCC/C=C\CCOC(=O)CCCCCOC(=O)C(COC(=O)NCc1ncc[nH]1)NC(=O)OCCCCCCCCOC(=O)C(CCCCCC)CCCCCCCC] |
| 953. | 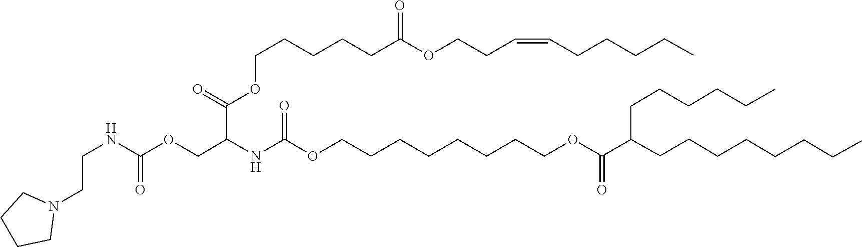 [SMILES: CCCCC/C=C\CCOC(=O)CCCCCOC(=O)C(COC(=O)NCCN1CCCC1)NC(=O)OCCCCCCCCOC(=O)C(CCCCCC)CCCCCCCC] |
| 954. | 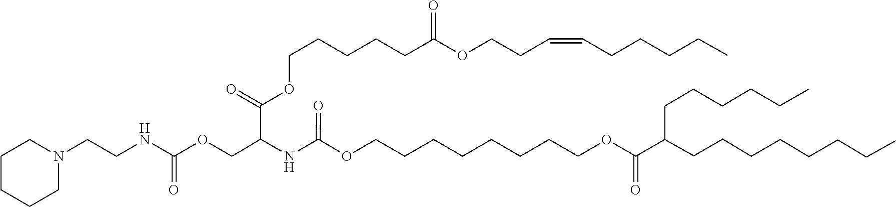 [SMILES: CCCCC/C=C\CCOC(=O)CCCCCOC(=O)C(COC(=O)NCCN1CCCCC1)NC(=O)OCCCCCCCCOC(=O)C(CCCCCC)CCCCCCCC] |
| 955. | 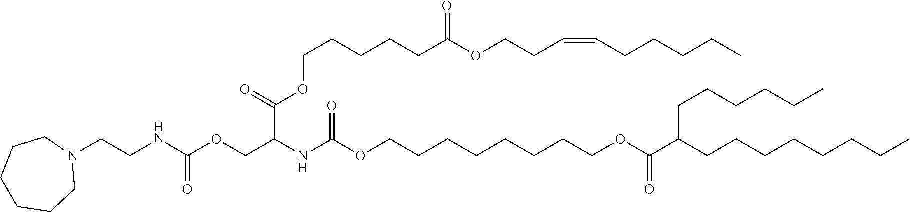 [SMILES: CCCCC/C=C\CCOC(=O)CCCCCOC(=O)C(COC(=O)NCCN1CCCCCC1)NC(=O)OCCCCCCCCOC(=O)C(CCCCCC)CCCCCCCC] |
| 956. | 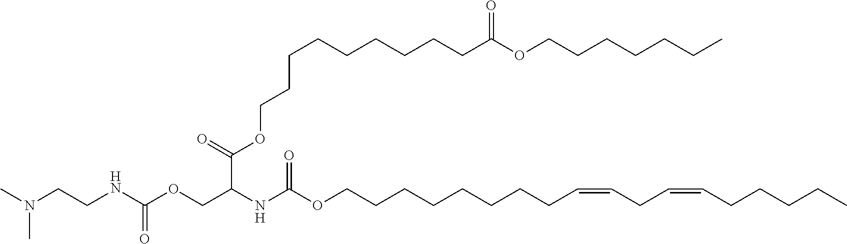 [SMILES: CCCCC/C=C\C/C=C\CCCCCCCCOC(=O)NC(COC(=O)NCCN(C)C)C(=O)OCCCCCCCCCC(=O)OCCCCCCC] |
| 957. | 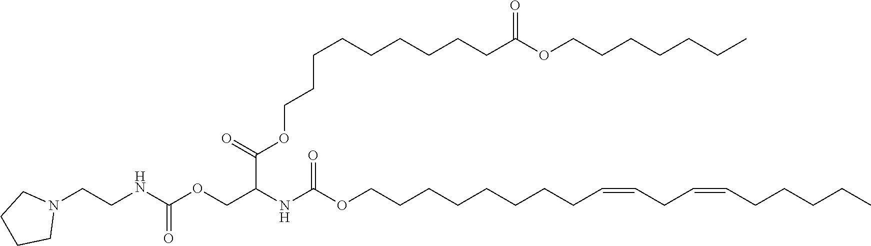 [SMILES: CCCCC/C=C\C/C=C\CCCCCCCCOC(=O)NC(COC(=O)NCCN1CCCC1)C(=O)OCCCCCCCCCC(=O)OCCCCCCC] |
| 958. | 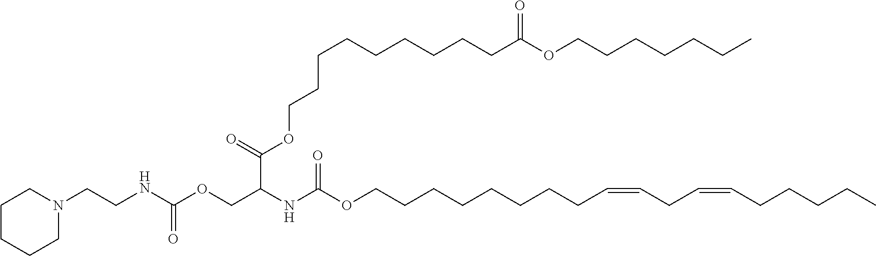 [SMILES: CCCCC/C=C\C/C=C\CCCCCCCCOC(=O)NC(COC(=O)NCCN1CCCCC1)C(=O)OCCCCCCCCCC(=O)OCCCCCCC] |
| 959. | 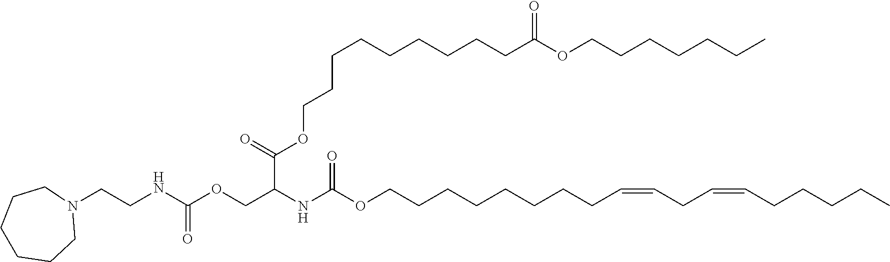 [SMILES: CCCCC/C=C\C/C=C\CCCCCCCCOC(=O)NC(COC(=O)NCCN1CCCCCC1)C(=O)OCCCCCCCCCC(=O)OCCCCCCC] |
| 960. | 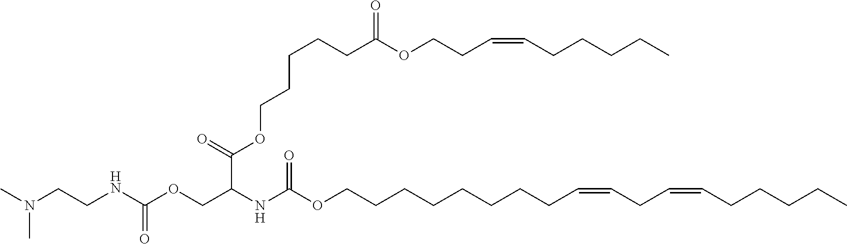 [SMILES: CCCCC/C=C\C/C=C\CCCCCCCCOC(=O)NC(COC(=O)NCCN(C)C)C(=O)OCCCCCC(=O)OCC/C=C\CCCCC] |
| 961. | 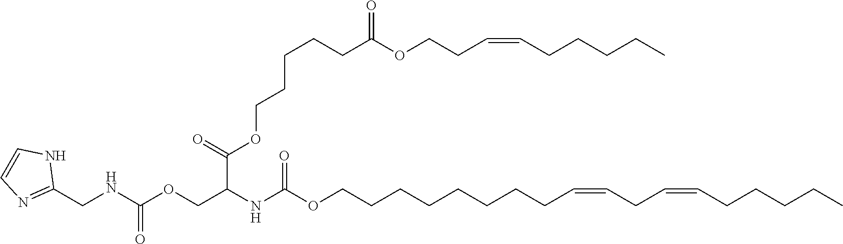 [SMILES: CCCCC/C=C\C/C=C\CCCCCCCCOC(=O)NC(COC(=O)NCc1ncc[nH]1)C(=O)OCCCCCC(=O)OCC/C=C\CCCCC] |
| 962. | 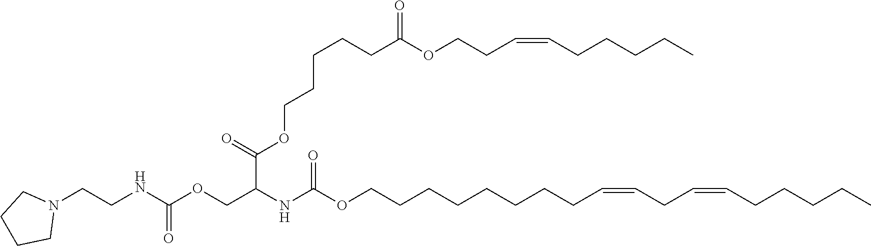 [SMILES: CCCCC/C=C\C/C=C\CCCCCCCCOC(=O)NC(COC(=O)NCCN1CCCC1)C(=O)OCCCCCC(=O)OCC/C=C\CCCCC] |
| 963. | 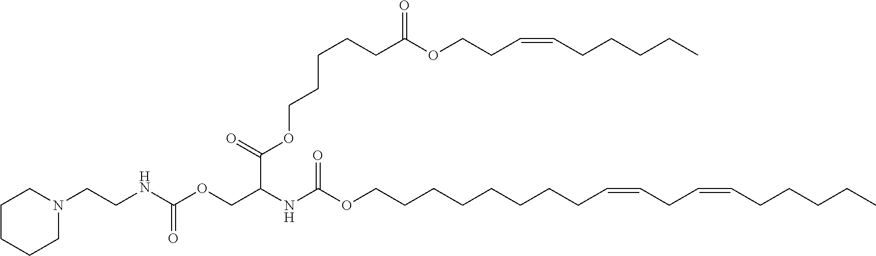 [SMILES: CCCCC/C=C\C/C=C\CCCCCCCCOC(=O)NC(COC(=O)NCCN1CCCCC1)C(=O)OCCCCCC(=O)OCC/C=C\CCCCC] |
| 964. | 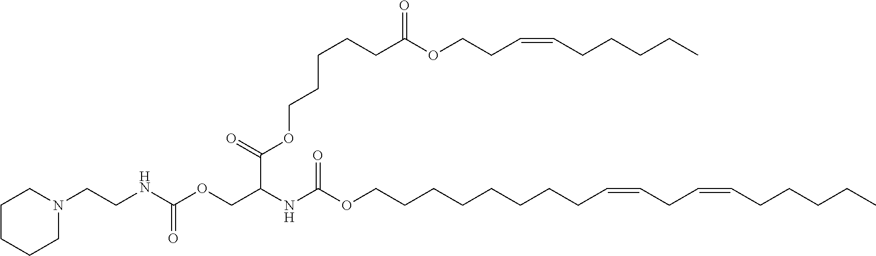 [SMILES: CCCCC/C=C\C/C=C\CCCCCCCCOC(=O)NC(COC(=O)NCCN1CCCCC1)C(=O)OCCCCCC(=O)OCC/C=C\CCCCC] |
| 965. | 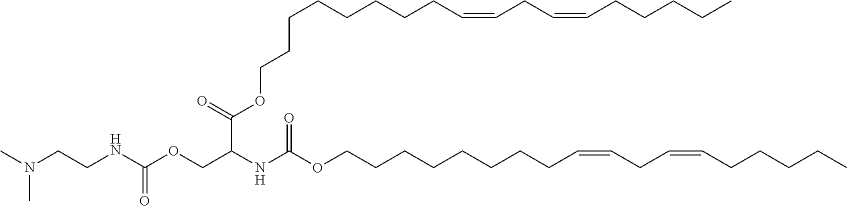 [SMILES: CCCCC/C=C\C/C=C\CCCCCCCCOC(=O)NC(COC(=O)NCCN(C)C)C(=O)OCCCCCCCC/C=C\C/C=C\CCCCC] |
| 966. | 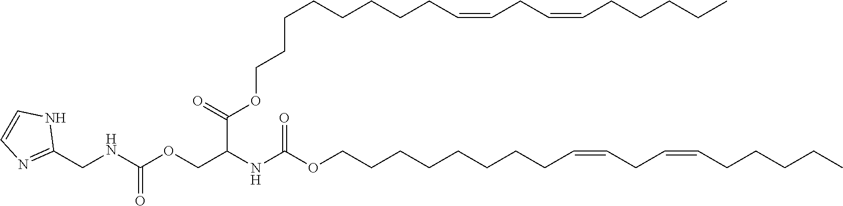 [SMILES: CCCCC/C=C\C/C=C\CCCCCCCCOC(=O)NC(COC(=O)NCc1ncc[nH]1)C(=O)OCCCCCCCC/C=C\C/C=C\CCCCC] |
| 967. | 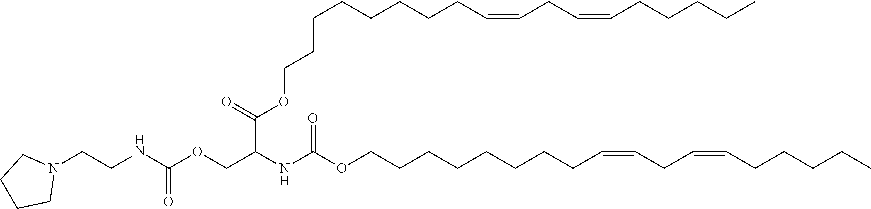 |
| 968. | 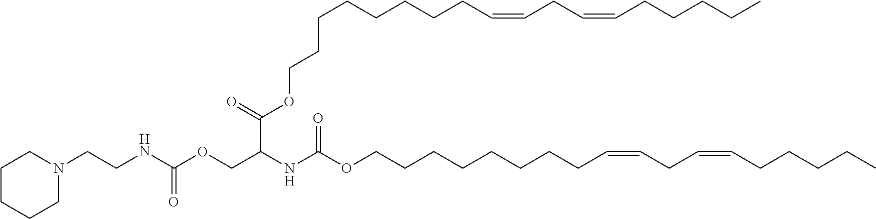 |
| 969. | 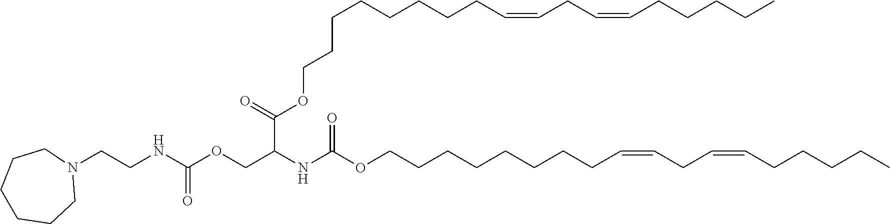 |
| 970. | 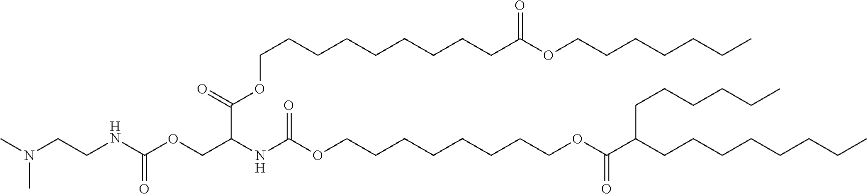 |
| 971. | 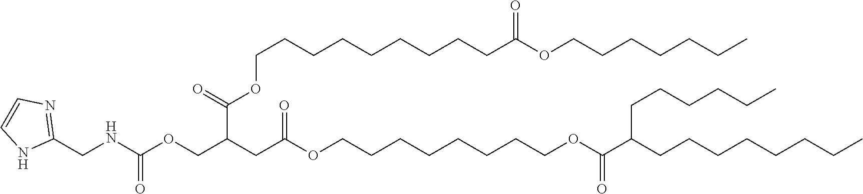 [SMILES: CCCCCCCCC(CCCCCC)C(=O)OCCCCCCCCOC(=O)CC(COC(=O)NCc1ncc[nH]1)C(=O)OCCCCCCCCCC(=O)OCCCCCCC] |
| 972. | 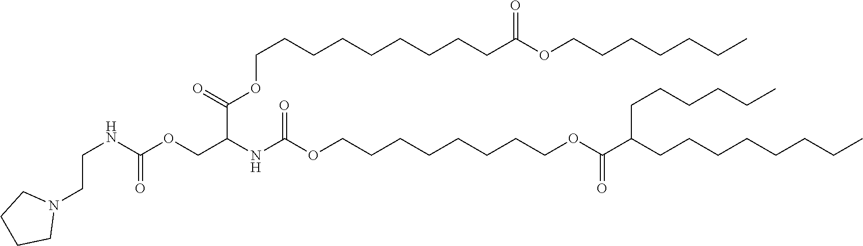 [SMILES: CCCCCCCCC(CCCCCC)C(=O)OCCCCCCCCOC(=O)NC(COC(=O)NCCN1CCCC1)C(=O)OCCCCCCCCCC(=O)OCCCCCCC] |
| 973. | 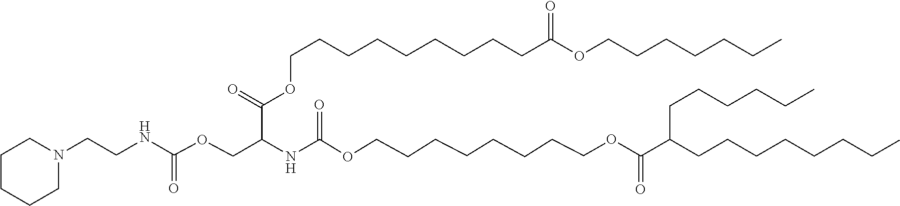 [SMILES: CCCCCCCCC(CCCCCC)C(=O)OCCCCCCCCOC(=O)NC(COC(=O)NCCN1CCCCC1)C(=O)OCCCCCCCCCC(=O)OCCCCCCC] |
| 974. | 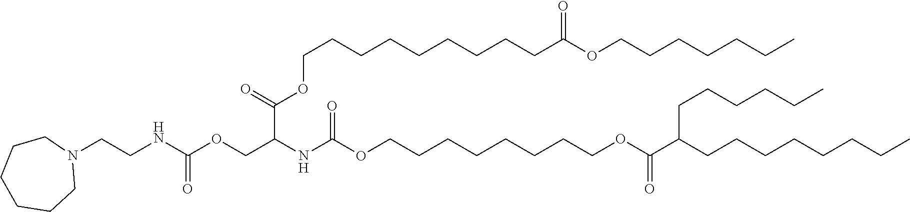 [SMILES: CCCCCCCCC(CCCCCC)C(=O)OCCCCCCCCOC(=O)NC(COC(=O)NCCN1CCCCCC1)C(=O)OCCCCCCCCCC(=O)OCCCCCCC] |
| 975. | 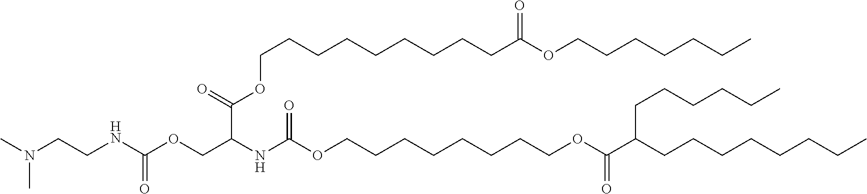 [SMILES: CCCCCCCCC(CCCCCC)C(=O)OCCCCCCCCOC(=O)NC(COC(=O)NCCN(C)C)C(=O)OCCCCCCCCCC(=O)OCCCCCCC] |
| 976. | 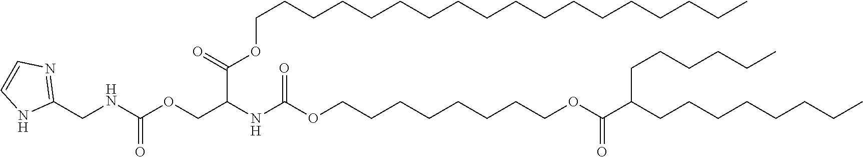 [SMILES: CCCCCCCCCCCCCCCCCCOC(=O)C(COC(=O)NCc1ncc[nH]1)NC(=O)OCCCCCCCCOC(=O)C(CCCCCC)CCCCCCCC] |
| 977. | 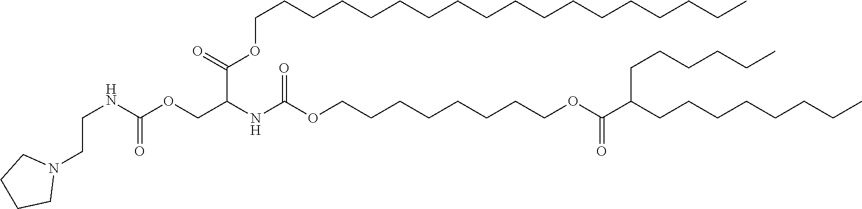 [SMILES: CCCCCCCCCCCCCCCCCCOC(=O)C(COC(=O)NCCN1CCCC1)NC(=O)OCCCCCCCCOC(=O)C(CCCCCC)CCCCCCCC] |
| 978. | 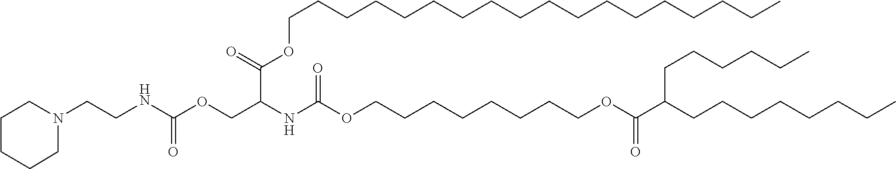 [SMILES: CCCCCCCCCCCCCCCCCCOC(=O)C(COC(=O)NCCN1CCCCC1)NC(=O)OCCCCCCCCOC(=O)C(CCCCCC)CCCCCCCC] |
| 979. | 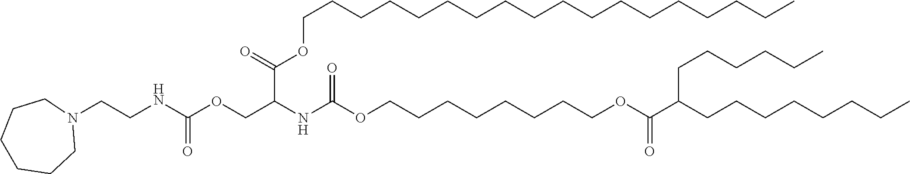 [SMILES: CCCCCCCCCCCCCCCCCCOC(=O)C(COC(=O)NCCN1CCCCCC1)NC(=O)OCCCCCCCCOC(=O)C(CCCCCC)CCCCCCCC] |
| 988. | 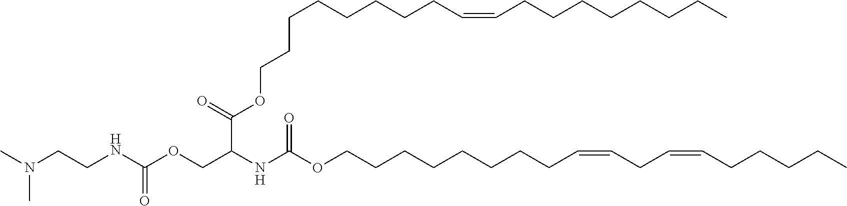 [SMILES: CCCCC/C=C\C/C=C\CCCCCCCCOC(=O)NC(COC(=O)NCCN(C)C)C(=O)OCCCCCCCC/C=C\CCCCCCCC] |
| 989. | 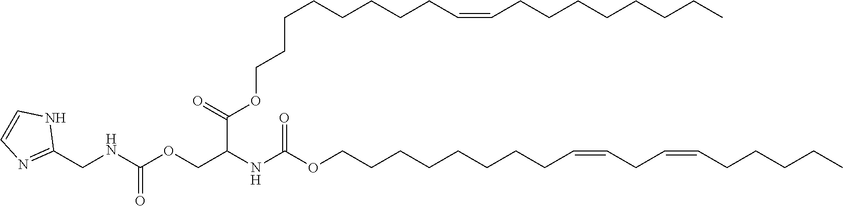 [SMILES: CCCCC/C=C\C/C=C\CCCCCCCCOC(=O)NC(COC(=O)NCc1ncc[nH]1)C(=O)OCCCCCCCC/C=C\CCCCCCCC] |
| 990. | 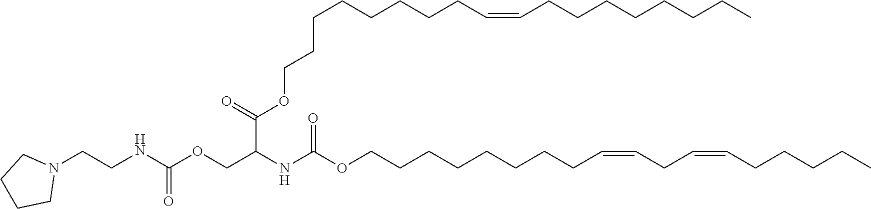 [SMILES: CCCCC/C=C\C/C=C\CCCCCCCCOC(=O)NC(COC(=O)NCCN1CCCC1)C(=O)OCCCCCCCC/C=C\CCCCCCCC] |
| 991. | 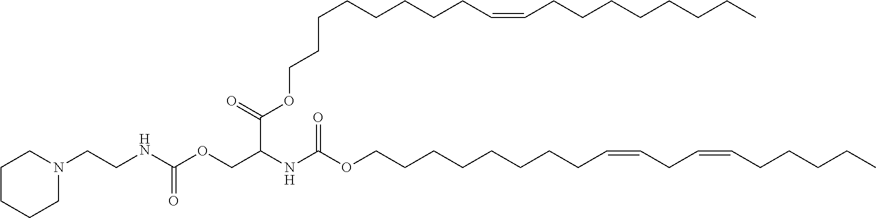 |
| 992. | 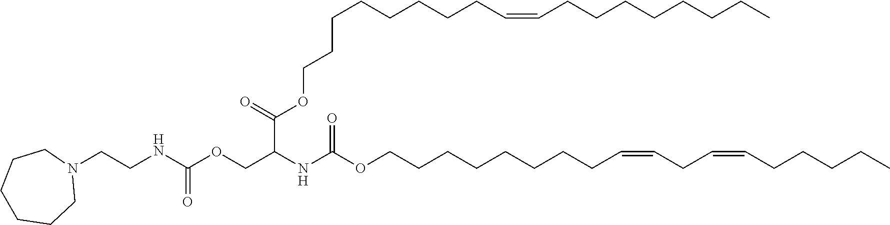 |
| 993. | 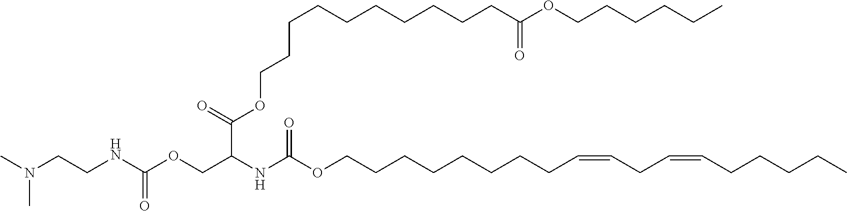 |
| 994. | 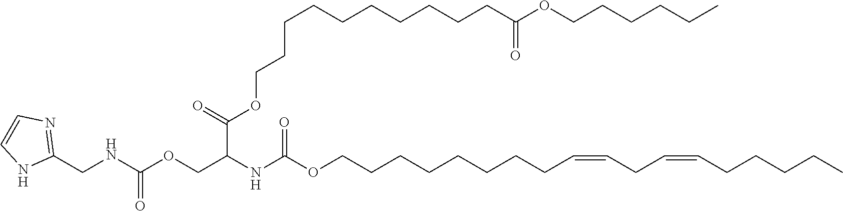 |
| 995. | 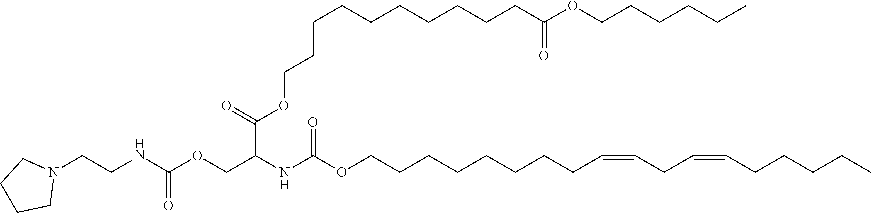 |
| 996. | 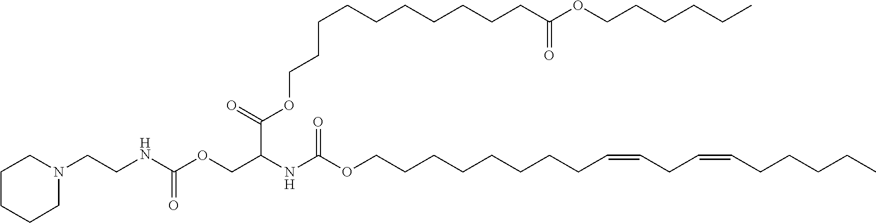 |
| 997. | 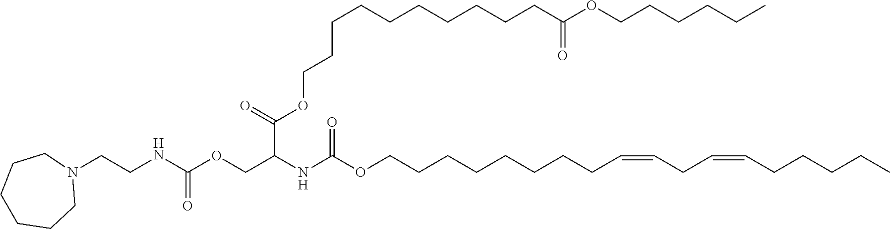 [SMILES: CCCCC/C=C\C/C=C\CCCCCCCCOC(=O)NC(COC(=O)NCCN1CCCCCC1)C(=O)OCCCCCCCCCCC(=O)OCCCCCC] |
| 998. | 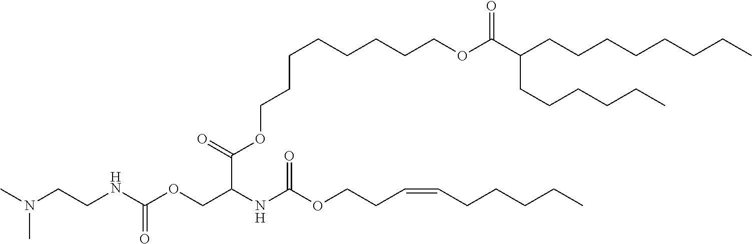 [SMILES: CCCCC/C=C\CCOC(=O)NC(COC(=O)NCCN(C)C)C(=O)OCCCCCCCCOC(=O)C(CCCCCC)CCCCCCCC] |
| 999. | 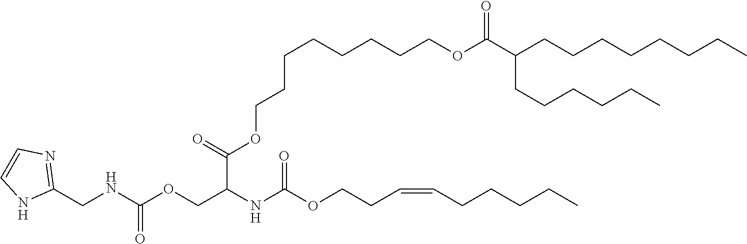 [SMILES: CCCCC/C=C\CCOC(=O)NC(COC(=O)NCc1ncc[nH]1)C(=O)OCCCCCCCCOC(=O)C(CCCCCC)CCCCCCCC] |
| 1000. | 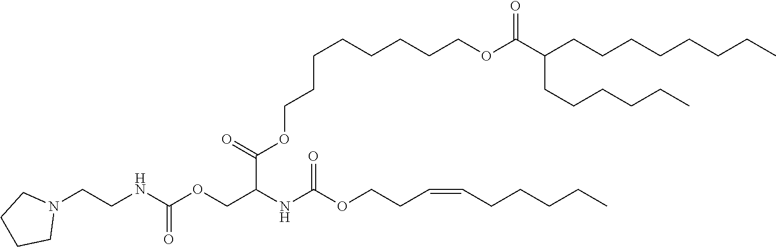 [SMILES: CCCCC/C=C\CCOC(=O)NC(COC(=O)NCCN1CCCC1)C(=O)OCCCCCCCCOC(=O)C(CCCCCC)CCCCCCCC] |
| 1001. | 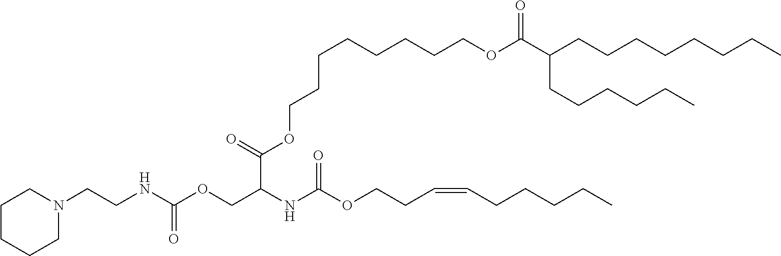 [SMILES: CCCCC/C=C\CCOC(=O)NC(COC(=O)NCCN1CCCCC1)C(=O)OCCCCCCCCOC(=O)C(CCCCCC)CCCCCCCC] |
| 1002. | 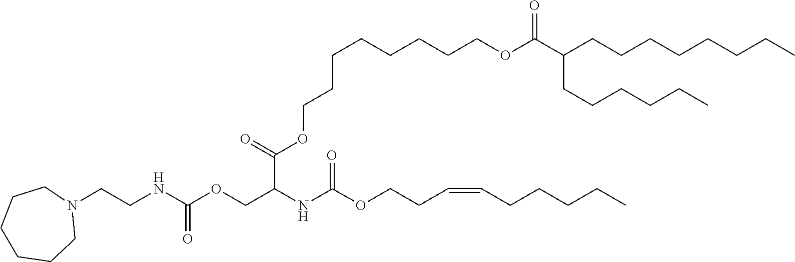 [SMILES: CCCCC/C=C\CCOC(=O)NC(COC(=O)NCCN1CCCCCC1)C(=O)OCCCCCCCCOC(=O)C(CCCCCC)CCCCCCCC] |
| 1003. | 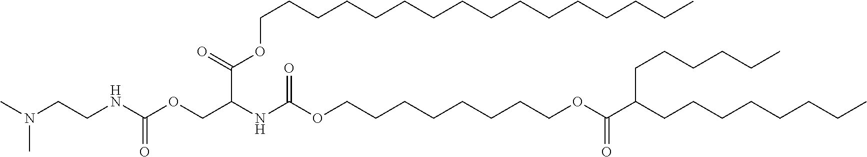 [SMILES: CCCCCCCCCCCCCCCCOC(=O)C(COC(=O)NCCN(C)C)NC(=O)OCCCCCCCCOC(=O)C(CCCCCC)CCCCCCCC] |
| 1004. | 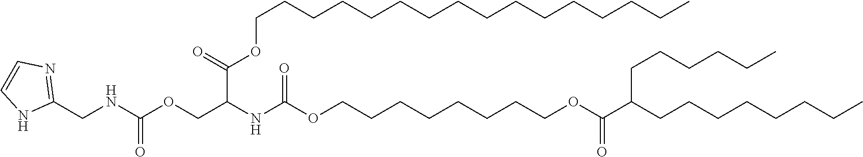 [SMILES: CCCCCCCCCCCCCCCCOC(=O)C(COC(=O)NCc1ncc[nH]1)NC(=O)OCCCCCCCCOC(=O)C(CCCCCC)CCCCCCCC] |
| 1005. | 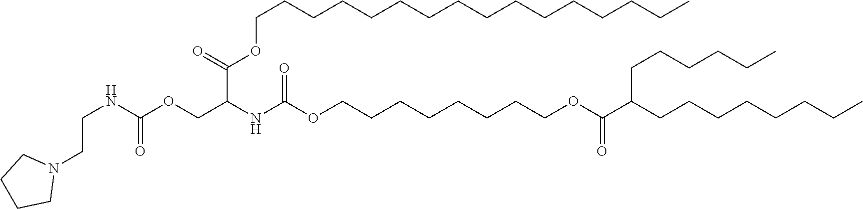 [SMILES: CCCCCCCCCCCCCCCCOC(=O)C(COC(=O)NCCN1CCCC1)NC(=O)OCCCCCCCCOC(=O)C(CCCCCC)CCCCCCCC] |
| 1006. | 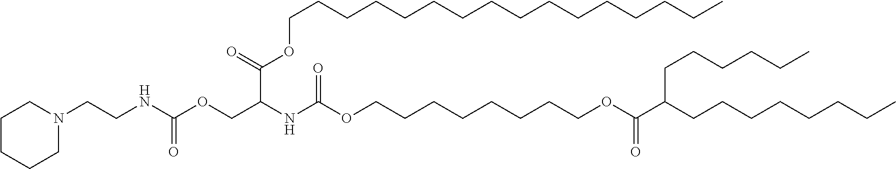 [SMILES: CCCCCCCCCCCCCCCCOC(=O)C(COC(=O)NCCN1CCCCC1)NC(=O)OCCCCCCCCOC(=O)C(CCCCCC)CCCCCCCC] |
| 1007. | 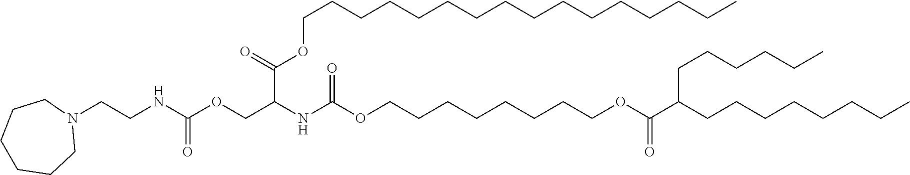 [SMILES: CCCCCCCCCCCCCCCCOC(=O)C(COC(=O)NCCN1CCCCCC1)NC(=O)OCCCCCCCCOC(=O)C(CCCCCC)CCCCCCCC] |
| 1008. | 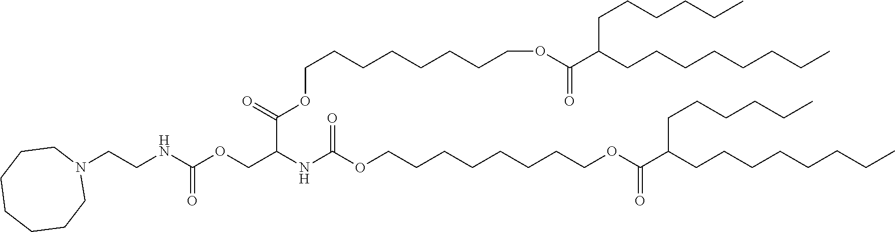 [SMILES: CCCCCCCCC(CCCCCC)C(=O)OCCCCCCCCOC(=O)NC(COC(=O)NCCN1CCCCCCC1)C(=O)OCCCCCCCCOC(=O)C(CCCCCC)CCCCCCCC] |
| 1009. | 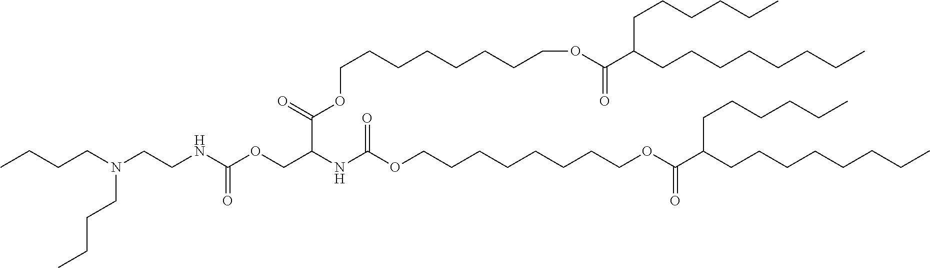 [SMILES: CCCCCCCCC(CCCCCC)C(=O)OCCCCCCCCOC(=O)NC(COC(=O)NCCN(CCCC)CCCC)C(=O)OCCCCCCCCOC(=O)C(CCCCCC)CCCCCCCC] |
| 1010. | 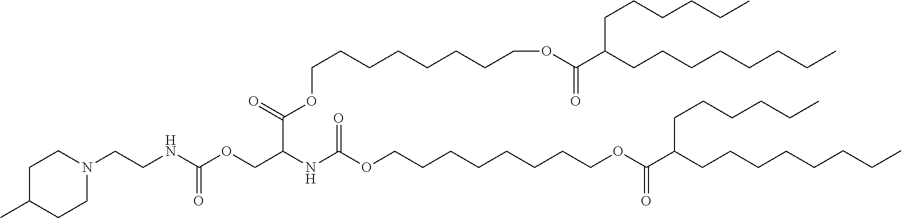 [SMILES: CCCCCCCCC(CCCCCC)C(=O)OCCCCCCCCOC(=O)NC(COC(=O)NCCN1CCC(C)CC1)C(=O)OCCCCCCCCOC(=O)C(CCCCCC)CCCCCCCC] |
| 1011. | 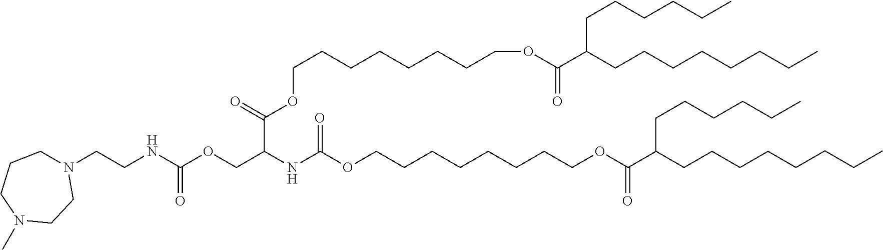 [SMILES: CCCCCCCCC(CCCCCC)C(=O)OCCCCCCCCOC(=O)NC(COC(=O)NCCN1CCCN(C)CC1)C(=O)OCCCCCCCCOC(=O)C(CCCCCC)CCCCCCCC] |
| 1012. | 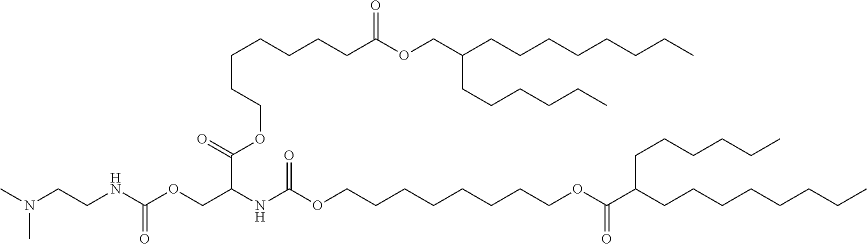 [SMILES: CCCCCCCCC(CCCCCC)COC(=O)CCCCCCCOC(=O)C(COC(=O)NCCN(C)C)NC(=O)OCCCCCCCCOC(=O)C(CCCCCC)CCCCCCCC] |
| 1013. | 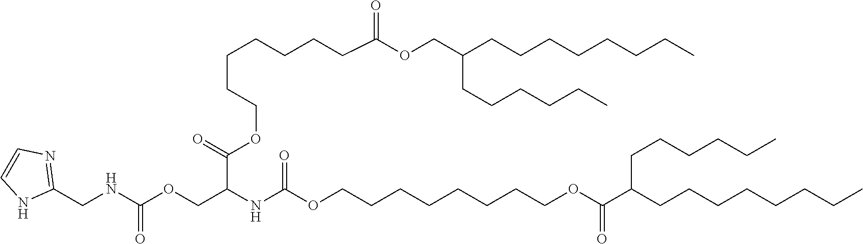 [SMILES: CCCCCCCCC(CCCCCC)COC(=O)CCCCCCCOC(=O)C(COC(=O)NCc1ncc[nH]1)NC(=O)OCCCCCCCCOC(=O)C(CCCCCC)CCCCCCCC] |
| 1014. | 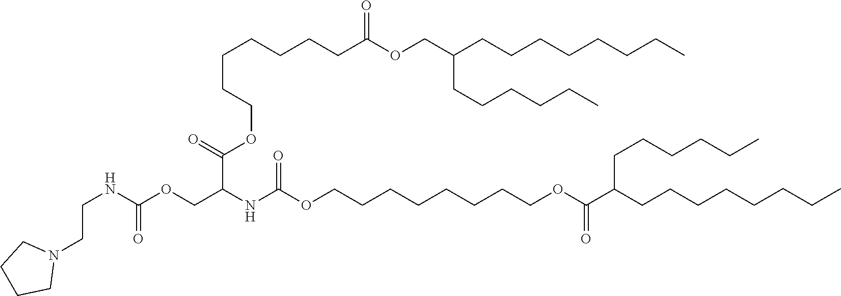 [SMILES: CCCCCCCCC(CCCCCC)COC(=O)CCCCCCCOC(=O)C(COC(=O)NCCN1CCCC1)NC(=O)OCCCCCCCCOC(=O)C(CCCCCC)CCCCCCCC] |
| 1015. | 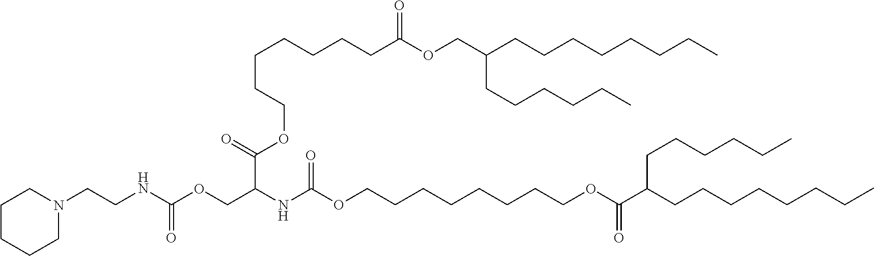 [SMILES: CCCCCCCCC(CCCCCC)COC(=O)CCCCCCCOC(=O)C(COC(=O)NCCN1CCCCC1)NC(=O)OCCCCCCCCOC(=O)C(CCCCCC)CCCCCCCC] |
| 1016. | 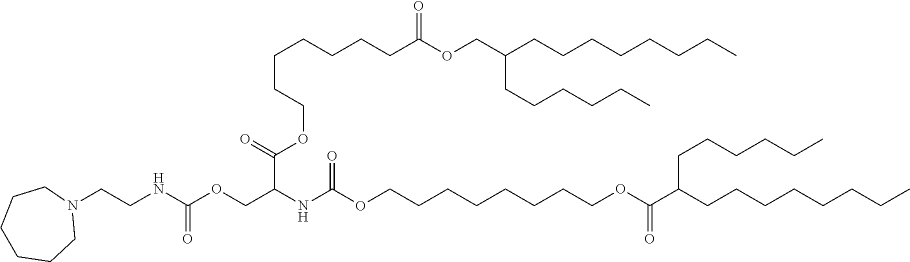 [SMILES: CCCCCCCCC(CCCCCC)COC(=O)CCCCCCCOC(=O)C(COC(=O)NCCN1CCCCCC1)NC(=O)OCCCCCCCCOC(=O)C(CCCCCC)CCCCCCCC] |
| 1017. | 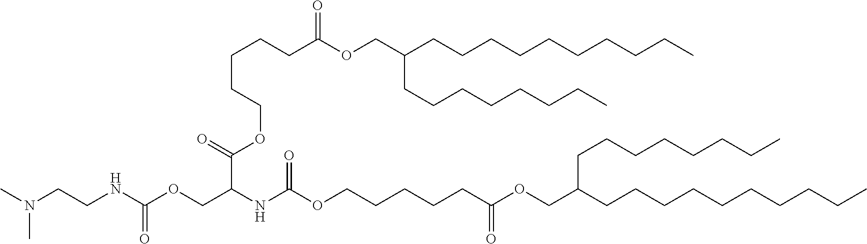 [SMILES: CCCCCCCCCCC(CCCCCCCC)COC(=O)CCCCCOC(=O)NC(COC(=O)NCCN(C)C)C(=O)OCCCCCC(=O)OCC(CCCCCCCC)CCCCCCCCCC] |
| 1018. | 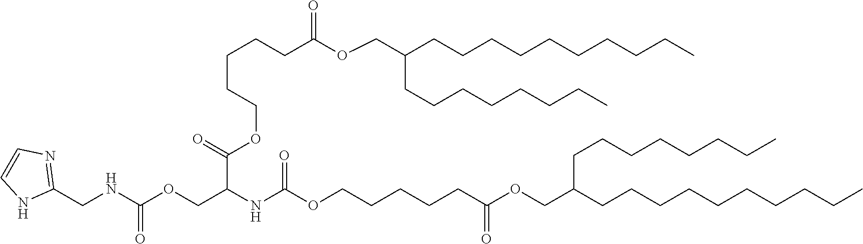 [SMILES: CCCCCCCCCCC(CCCCCCCC)COC(=O)CCCCCOC(=O)NC(COC(=O)NCc1ncc[nH]1)C(=O)OCCCCCC(=O)OCC(CCCCCCCC)CCCCCCCCCC] |
| 1019. | 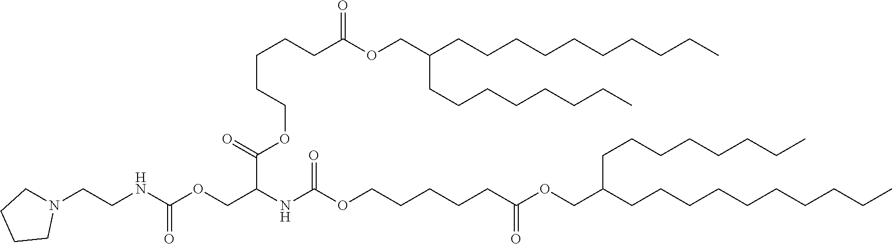 [SMILES: CCCCCCCCCCC(CCCCCCCC)COC(=O)CCCCCOC(=O)NC(COC(=O)NCCN1CCCC1)C(=O)OCCCCCC(=O)OCC(CCCCCCCC)CCCCCCCCCC] |
| 1020. | 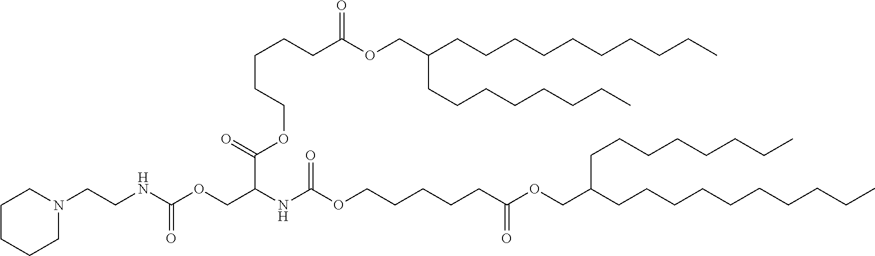 [SMILES: CCCCCCCCCCC(CCCCCCCC)COC(=O)CCCCCOC(=O)NC(COC(=O)NCCN1CCCCC1)C(=O)OCCCCCC(=O)OCC(CCCCCCCC)CCCCCCCCCC] |
| 1021. | 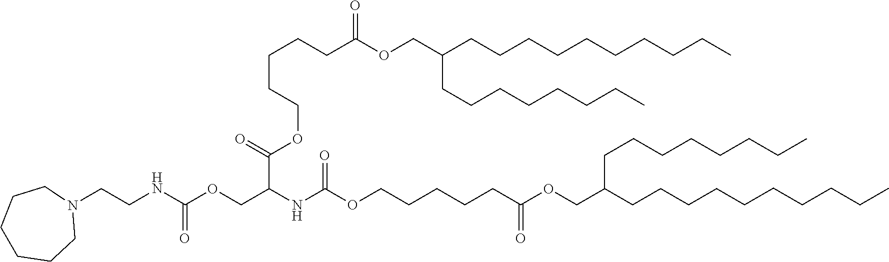 [SMILES: CCCCCCCCCCC(CCCCCCCC)COC(=O)CCCCCOC(=O)NC(COC(=O)NCCN1CCCCCC1)C(=O)OCCCCCC(=O)OCC(CCCCCCCC)CCCCCCCCCC] |
| 1022. | 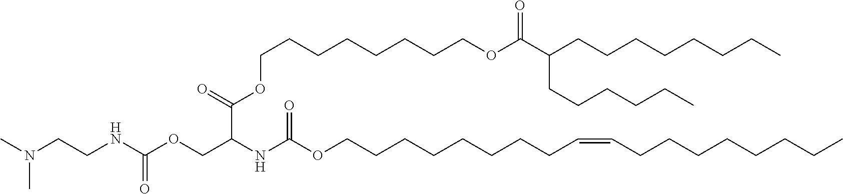 [SMILES: CCCCCCCC/C=C\CCCCCCCCOC(=O)NC(COC(=O)NCCN(C)C)C(=O)OCCCCCCCCOC(=O)C(CCCCCC)CCCCCCCC] |
| 1023. | 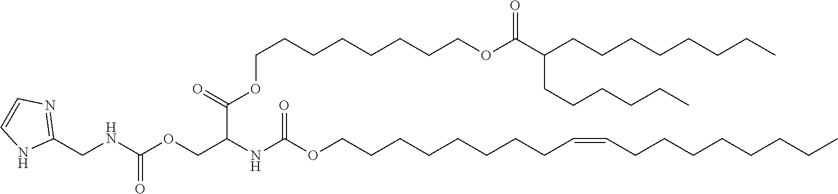 [SMILES: CCCCCCCC/C=C\CCCCCCCCOC(=O)NC(COC(=O)NCc1ncc[nH]1)C(=O)OCCCCCCCCOC(=O)C(CCCCCC)CCCCCCCC] |
| 1024. | 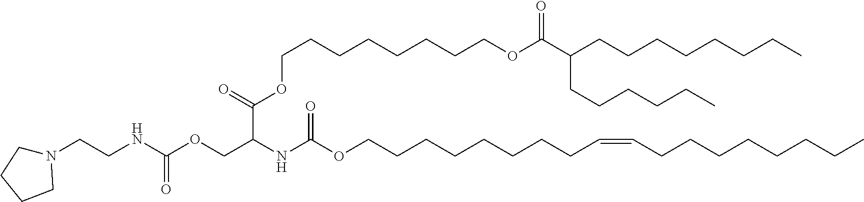 [SMILES: CCCCCCCC/C=C\CCCCCCCCOC(=O)NC(COC(=O)NCCN1CCCC1)C(=O)OCCCCCCCCOC(=O)C(CCCCCC)CCCCCCCC] |
| 1025. | 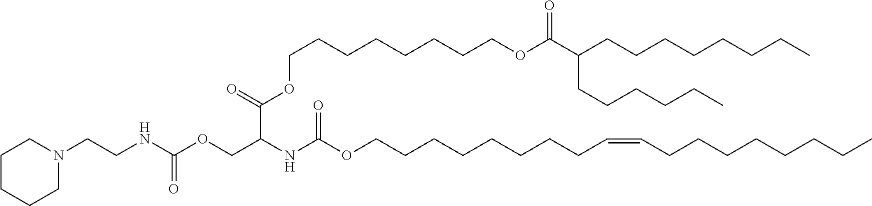 [SMILES: CCCCCCCC/C=C\CCCCCCCCOC(=O)NC(COC(=O)NCCN1CCCCC1)C(=O)OCCCCCCCCOC(=O)C(CCCCCC)CCCCCCCC] |
| 1026. | 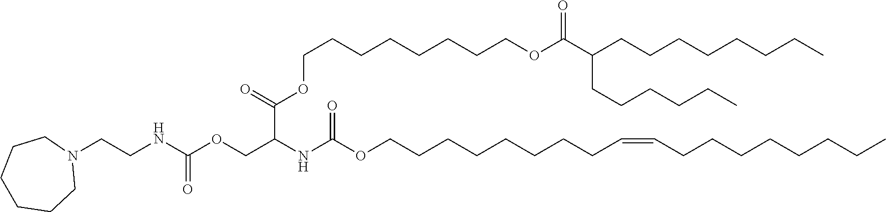 [SMILES: CCCCCCCC/C=C\CCCCCCCCOC(=O)NC(COC(=O)NCCN1CCCCCC1)C(=O)OCCCCCCCCOC(=O)C(CCCCCC)CCCCCCCC] |
| 1027. | 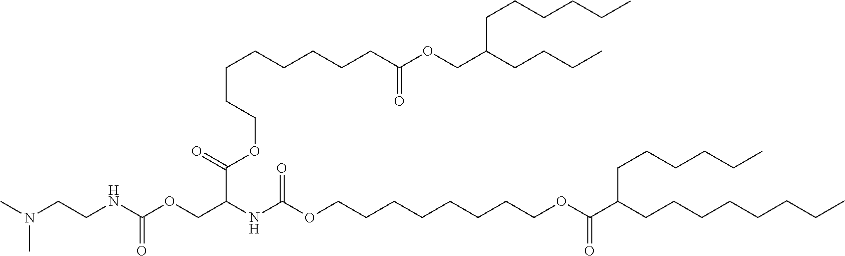 [SMILES: CCCCCCCCC(CCCCCC)C(=O)OCCCCCCCCOC(=O)NC(COC(=O)NCCN(C)C)C(=O)OCCCCCCCCC(=O)OCC(CCCC)CCCCCC] |
| 1028. | 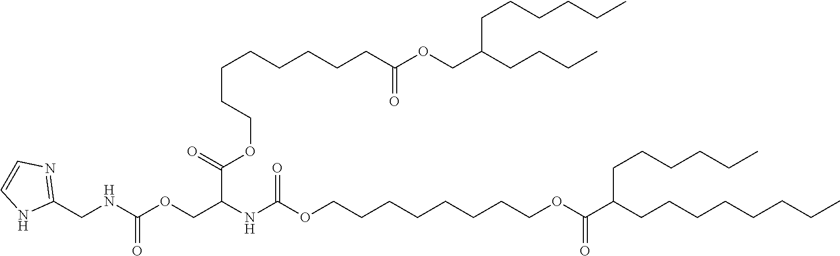 [SMILES: CCCCCCCCC(CCCCCC)C(=O)OCCCCCCCCOC(=O)NC(COC(=O)NCc1ncc[nH]1)C(=O)OCCCCCCCCC(=O)OCC(CCCC)CCCCCC] |
| 1029. | 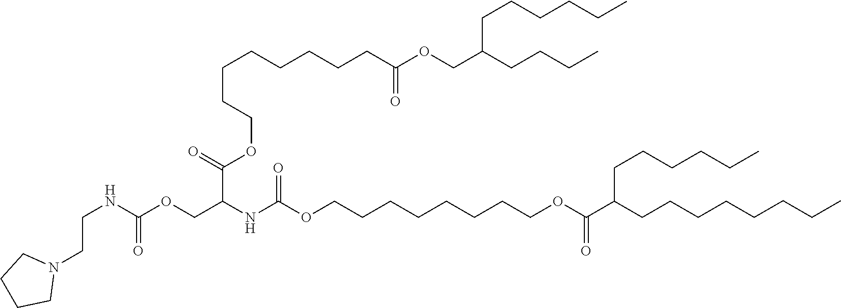 [SMILES: CCCCCCCCC(CCCCCC)C(=O)OCCCCCCCCOC(=O)NC(COC(=O)NCCN1CCCC1)C(=O)OCCCCCCCCC(=O)OCC(CCCC)CCCCCC] |
| 1030. | 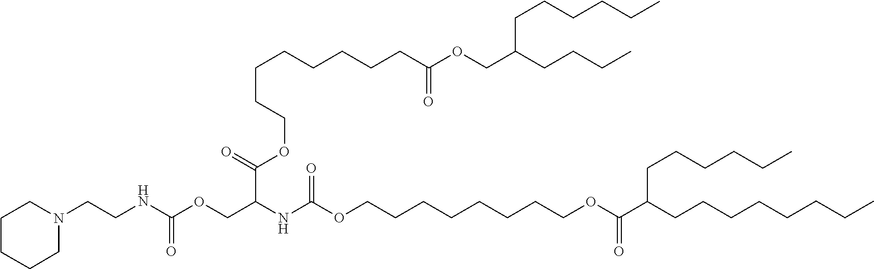 [SMILES: CCCCCCCCC(CCCCCC)C(=O)OCCCCCCCCOC(=O)NC(COC(=O)NCCN1CCCCC1)C(=O)OCCCCCCCCC(=O)OCC(CCCC)CCCCCC] |
| 1031. | 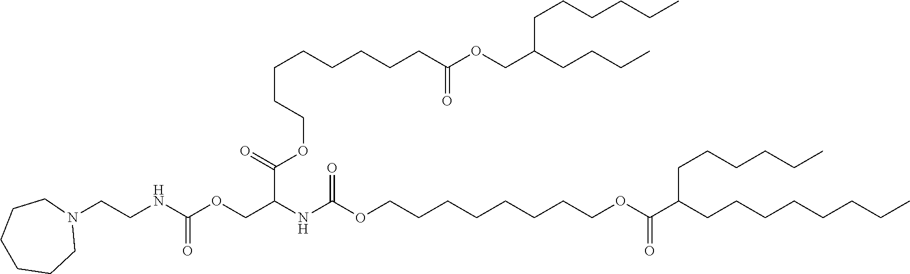 [SMILES: CCCCCCCCC(CCCCCC)C(=O)OCCCCCCCCOC(=O)NC(COC(=O)NCCN1CCCCCC1)C(=O)OCCCCCCCCC(=O)OCC(CCCC)CCCCCC] |
| 1032. | 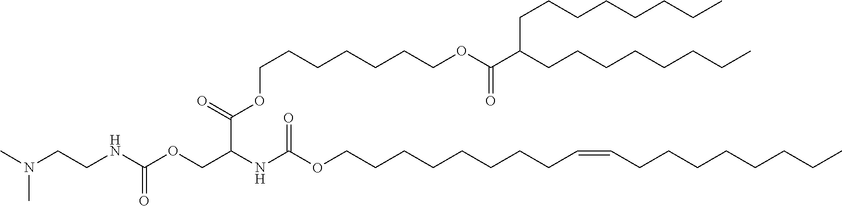 |
| 1033. | 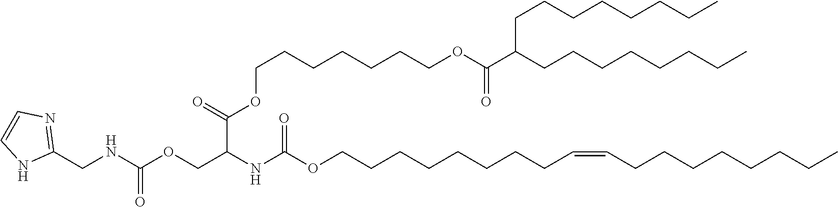 [SMILES: CCCCCCCC/C=C\CCCCCCCCOC(=O)NC(COC(=O)NCc1ncc[nH]1)C(=O)OCCCCCCCOC(=O)C(CCCCCCCC)CCCCCCCC] |
| 1034. | 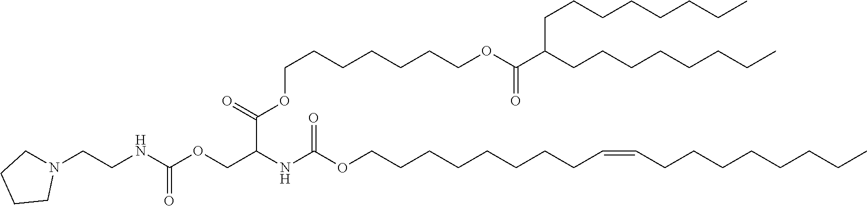 |
| 1035. | 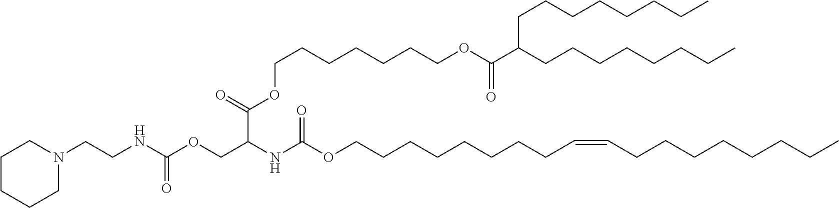 |
| 1036. | 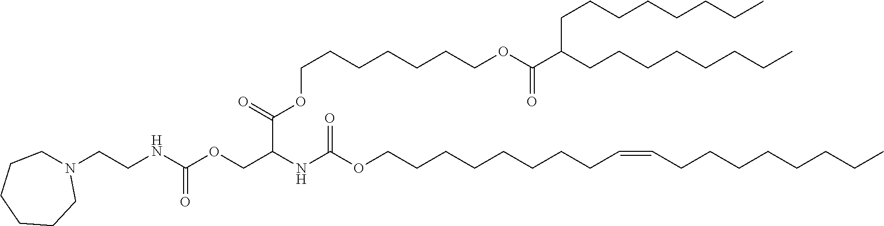 [SMILES: CCCCCCCC/C=C\CCCCCCCCOC(=O)NC(COC(=O)NCCN1CCCCCC1)C(=O)OCCCCCCCOC(=O)C(CCCCCCCC)CCCCCCCC] |
| 1037. | 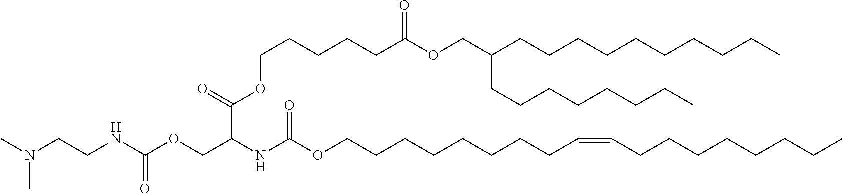 [SMILES: CCCCCCCC/C=C\CCCCCCCCOC(=O)NC(COC(=O)NCCN(C)C)C(=O)OCCCCCC(=O)OCC(CCCCCCCC)CCCCCCCCCC] |
| 1038. | 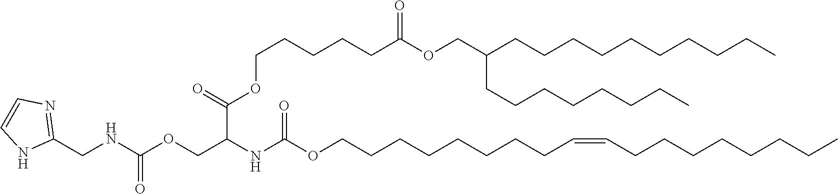 [SMILES: CCCCCCCC/C=C\CCCCCCCCOC(=O)NC(COC(=O)NCc1ncc[nH]1)C(=O)OCCCCCC(=O)OCC(CCCCCCCC)CCCCCCCCCC] |
| 1039. | 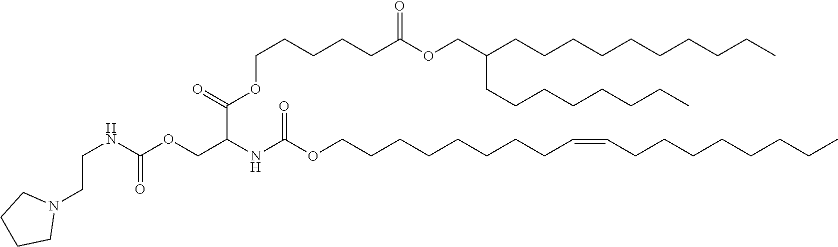 [SMILES: CCCCCCCC/C=C\CCCCCCCCOC(=O)NC(COC(=O)NCCN1CCCC1)C(=O)OCCCCCC(=O)OCC(CCCCCCCC)CCCCCCCCCC] |
| 1040. | 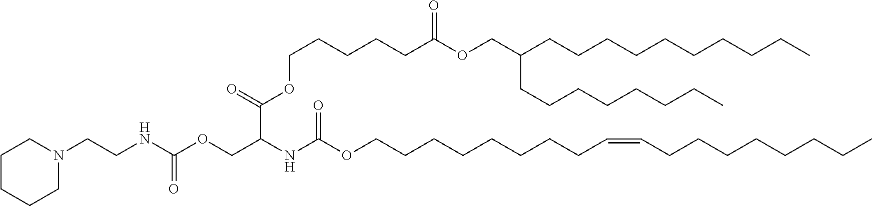 [SMILES: CCCCCCCC/C=C\CCCCCCCCOC(=O)NC(COC(=O)NCCN1CCCCC1)C(=O)OCCCCCC(=O)OCC(CCCCCCCC)CCCCCCCCCC] |
| 1041. | 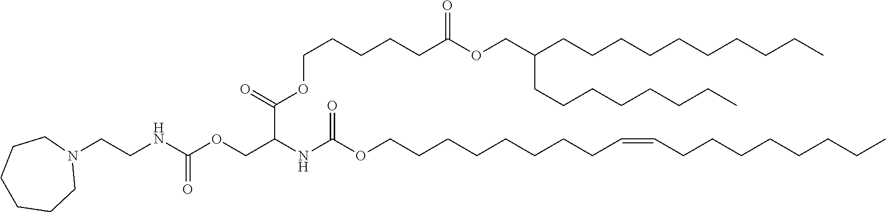 [SMILES: CCCCCCCC/C=C\CCCCCCCCOC(=O)NC(COC(=O)NCCN1CCCCCC1)C(=O)OCCCCCC(=O)OCC(CCCCCCCC)CCCCCCCCCC] |
| 1042. | 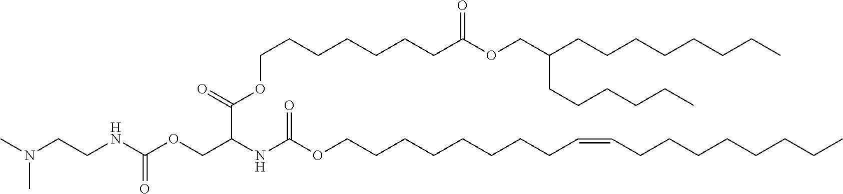 [SMILES: CCCCCCCC/C=C\CCCCCCCCOC(=O)NC(COC(=O)NCCN(C)C)C(=O)OCCCCCCCC(=O)OCC(CCCCCC)CCCCCCCC] |
| 1043. | 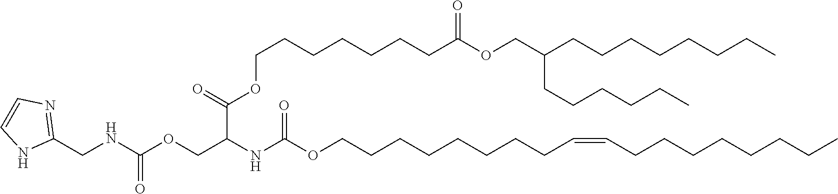 [SMILES: CCCCCCCC/C=C\CCCCCCCCOC(=O)NC(COC(=O)NCc1ncc[nH]1)C(=O)OCCCCCCCC(=O)OCC(CCCCCC)CCCCCCCC] |
| 1044. | 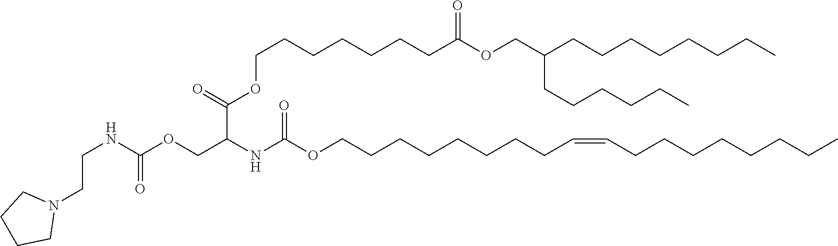 [SMILES: CCCCCCCC/C=C\CCCCCCCCOC(=O)NC(COC(=O)NCCN1CCCC1)C(=O)OCCCCCCCC(=O)OCC(CCCCCC)CCCCCCCC] |
| 1045. | 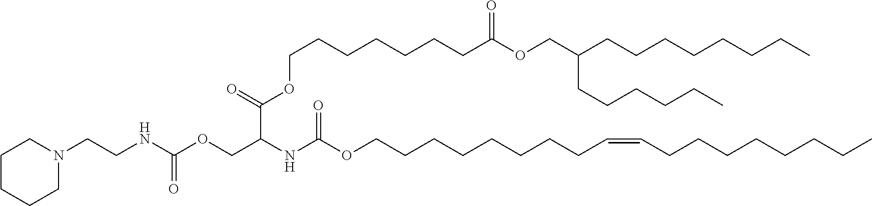 [SMILES: CCCCCCCC/C=C\CCCCCCCCOC(=O)NC(COC(=O)NCCN1CCCCC1)C(=O)OCCCCCCCC(=O)OCC(CCCCCC)CCCCCCCC] |
| 1046. | 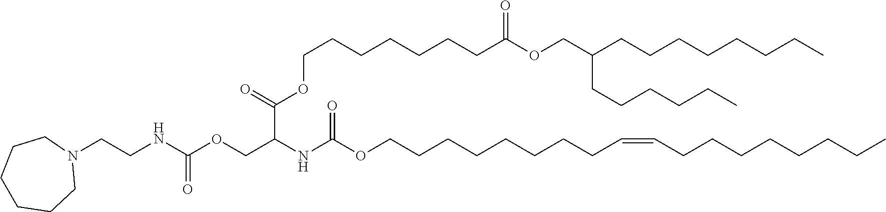 [SMILES: CCCCCCCC/C=C\CCCCCCCCOC(=O)NC(COC(=O)NCCN1CCCCCC1)C(=O)OCCCCCCCC(=O)OCC(CCCCCC)CCCCCCCC] |
| 1047. | 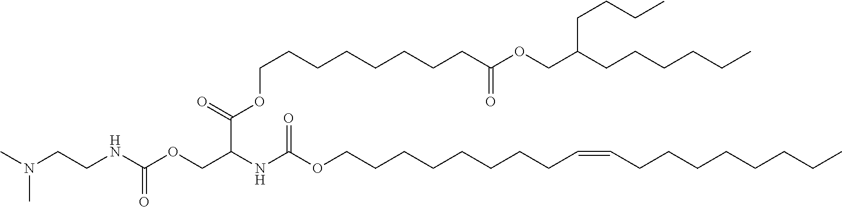 |
| 1048. | 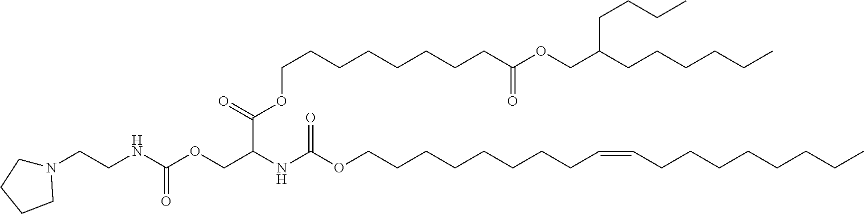 |
| 1049. | 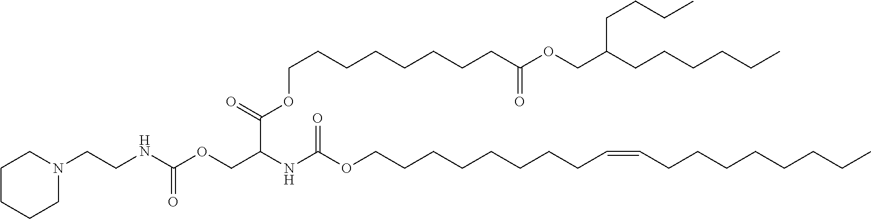 |
| 1069. | 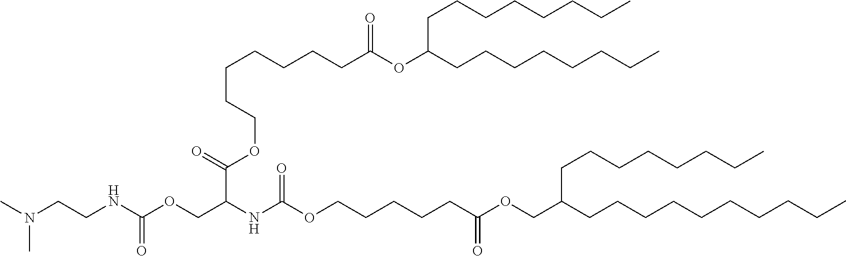 [SMILES: CCCCCCCCCCC(CCCCCCCC)COC(=O)CCCCCOC(=O)NC(COC(=O)NCCN(C)C)C(=O)OCCCCCCCC(=O)OC(CCCCCCCC)CCCCCCCC] |
| 1070. | 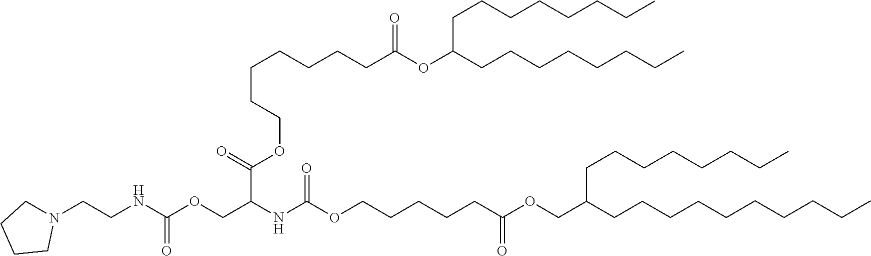 [SMILES: CCCCCCCCCCC(CCCCCCCC)COC(=O)CCCCCOC(=O)NC(COC(=O)NCCN1CCCC1)C(=O)OCCCCCCCC(=O)OC(CCCCCCCC)CCCCCCCC] |
| 1071. | 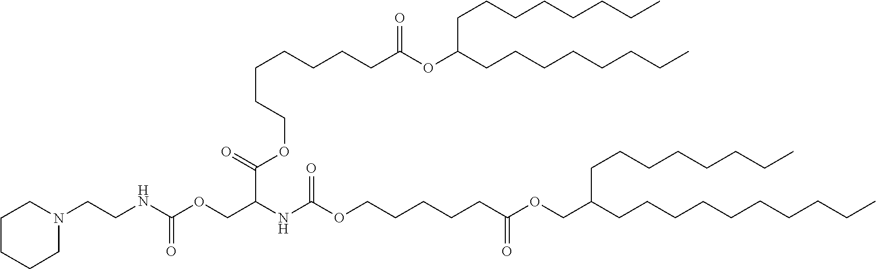 [SMILES: CCCCCCCCCCC(CCCCCCCC)COC(=O)CCCCCOC(=O)NC(COC(=O)NCCN1CCCCC1)C(=O)OCCCCCCCC(=O)OC(CCCCCCCC)CCCCCCCC] |
| 1072. | 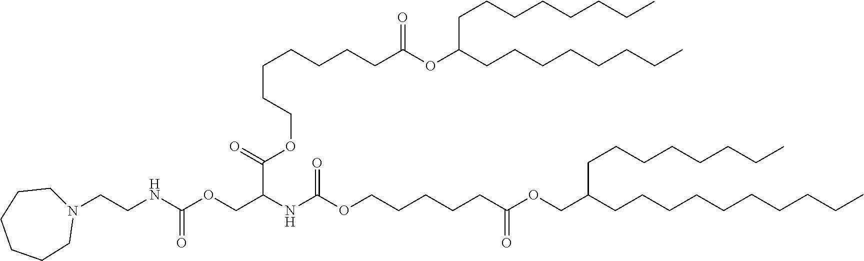 [SMILES: CCCCCCCCCCC(CCCCCCCC)COC(=O)CCCCCOC(=O)NC(COC(=O)NCCN1CCCCCC1)C(=O)OCCCCCCCC(=O)OC(CCCCCCCC)CCCCCCCC] |
| 1084. | 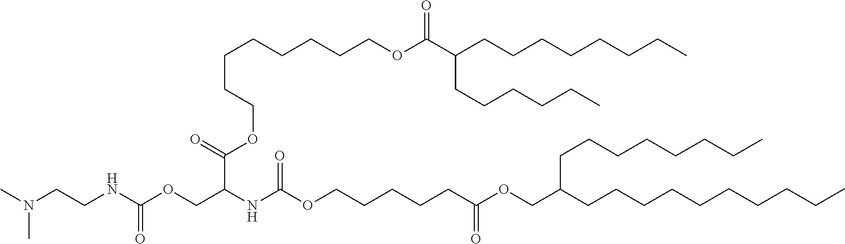 [SMILES: CCCCCCCCCCC(CCCCCCCC)COC(=O)CCCCCOC(=O)NC(COC(=O)NCCN(C)C)C(=O)OCCCCCCCCOC(=O)C(CCCCCC)CCCCCCCC] |
| 1085. | 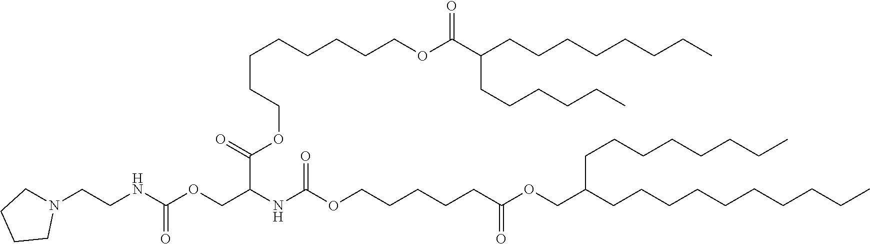 [SMILES: CCCCCCCCCCC(CCCCCCCC)COC(=O)CCCCCOC(=O)NC(COC(=O)NCCN1CCCC1)C(=O)OCCCCCCCCOC(=O)C(CCCCCC)CCCCCCCC] |
| 1086. | 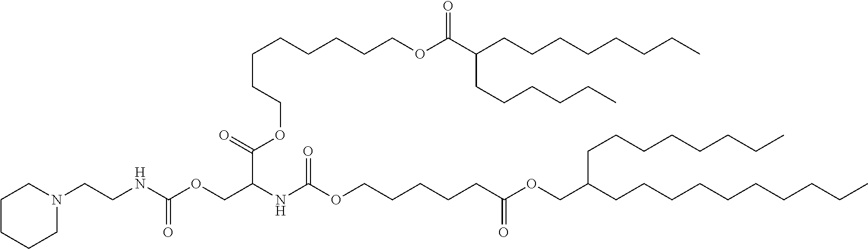 [SMILES: CCCCCCCCCCC(CCCCCCCC)COC(=O)CCCCCOC(=O)NC(COC(=O)NCCN1CCCCC1)C(=O)OCCCCCCCCOC(=O)C(CCCCCC)CCCCCCCC] |
| 1087. | 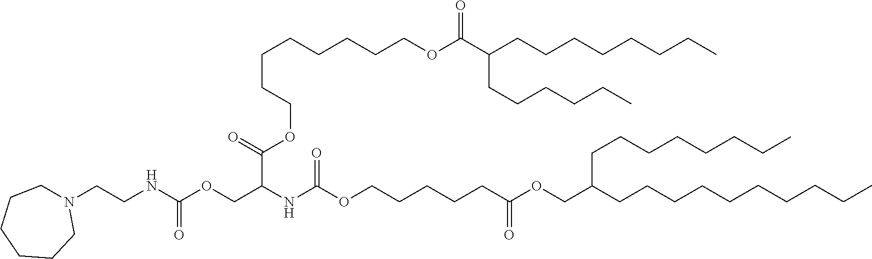 [SMILES: CCCCCCCCCCC(CCCCCCCC)COC(=O)CCCCCOC(=O)NC(COC(=O)NCCN1CCCCCC1)C(=O)OCCCCCCCCOC(=O)C(CCCCCC)CCCCCCCC] |
| 1088. | 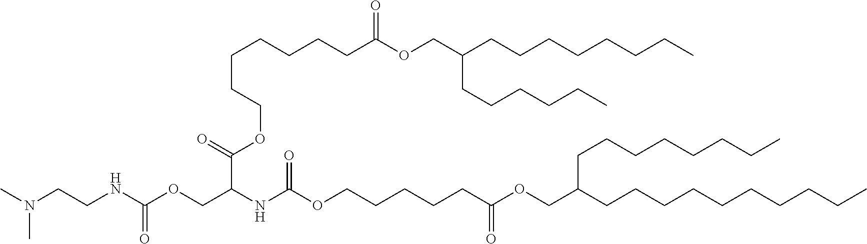 [SMILES: CCCCCCCCCCC(CCCCCCCC)COC(=O)CCCCCOC(=O)NC(COC(=O)NCCN(C)C)C(=O)OCCCCCCCC(=O)OCC(CCCCCC)CCCCCCCC] |
| 1089. | 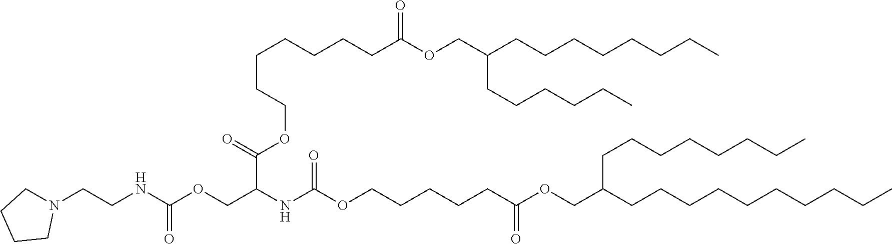 [SMILES: CCCCCCCCCCC(CCCCCCCC)COC(=O)CCCCCOC(=O)NC(COC(=O)NCCN1CCCC1)C(=O)OCCCCCCCC(=O)OCC(CCCCCC)CCCCCCCC] |
| 1090. | 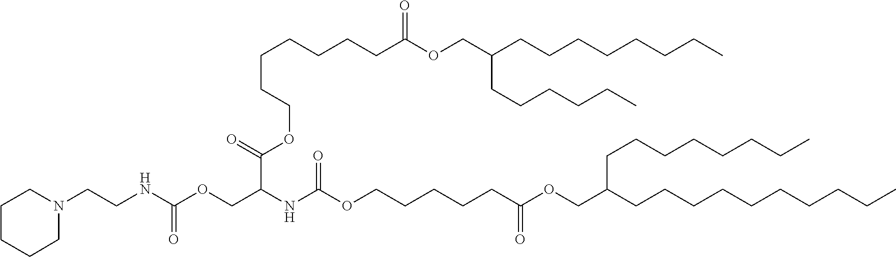 [SMILES: CCCCCCCCCCC(CCCCCCCC)COC(=O)CCCCCOC(=O)NC(COC(=O)NCCN1CCCCC1)C(=O)OCCCCCCCC(=O)OCC(CCCCCC)CCCCCCCC] |
| 1091. | 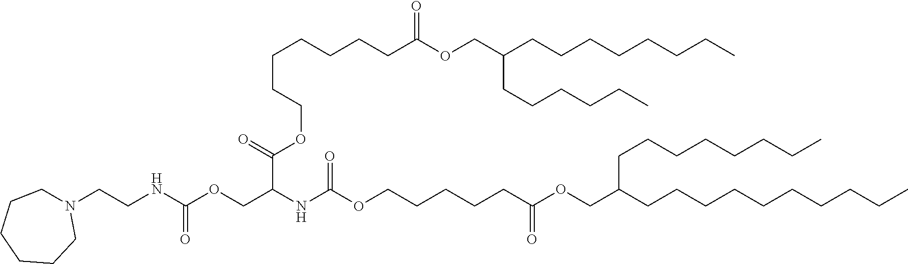 [SMILES: CCCCCCCCCCC(CCCCCCCC)COC(=O)CCCCCOC(=O)NC(COC(=O)NCCN1CCCCCC1)C(=O)OCCCCCCCC(=O)OCC(CCCCCC)CCCCCCCC] |
| 1092. | 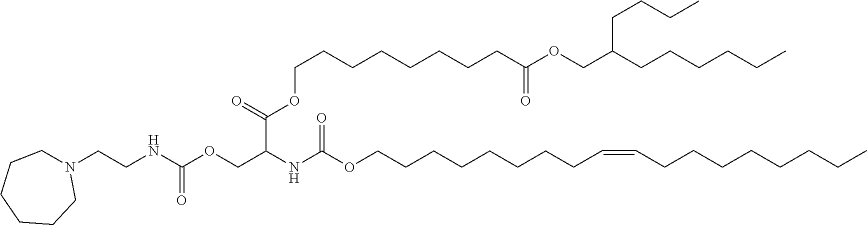 [SMILES: CCCCCCCC/C=C\CCCCCCCCOC(=O)NC(COC(=O)NCCN1CCCCCC1)C(=O)OCCCCCCCCC(=O)OCC(CCCC)CCCCCC] |
| 1098. | 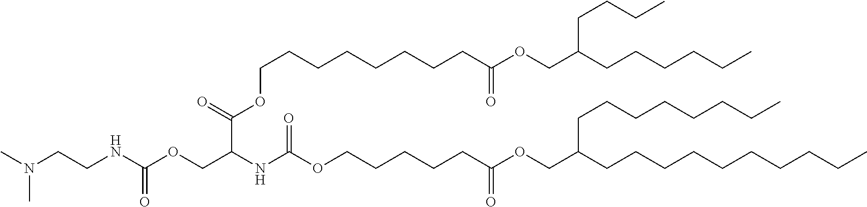 |
| 1099. | 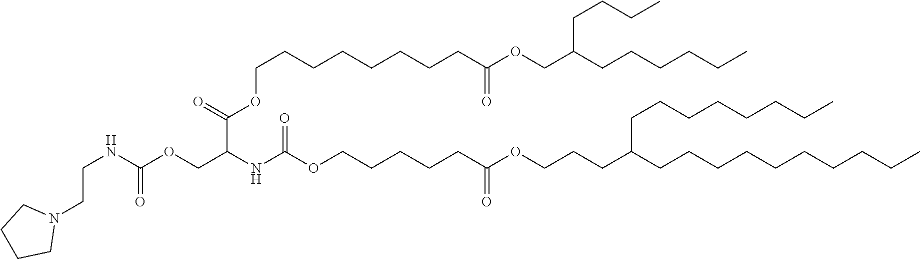 [SMILES: CCCCCCCCCCC(CCCCCCCC)CCCOC(=O)CCCCCOC(=O)NC(COC(=O)NCCN1CCCC1)C(=O)OCCCCCCCCC(=O)OCC(CCCC)CCCCCC] |
| 1100. | 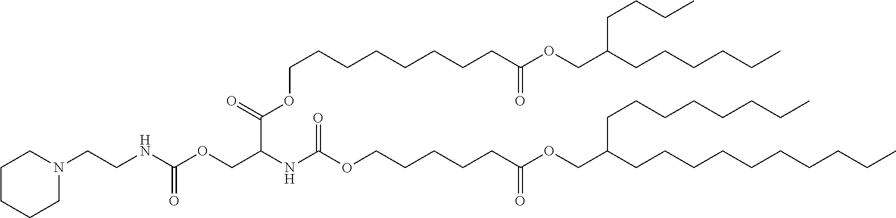 |
| 1101. | 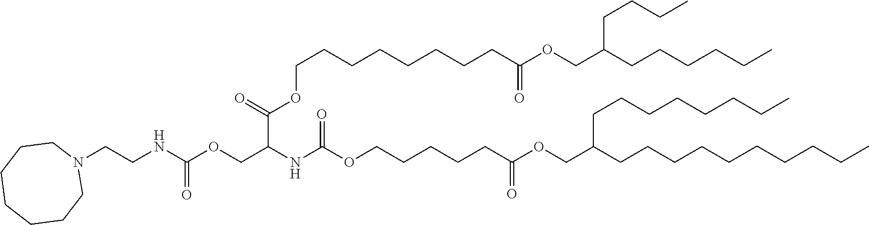 [SMILES: CCCCCCCCCCC(CCCCCCCC)COC(=O)CCCCCOC(=O)NC(COC(=O)NCCN1CCCCCCC1)C(=O)OCCCCCCCCC(=O)OCC(CCCC)CCCCCC] |
| 1117. | 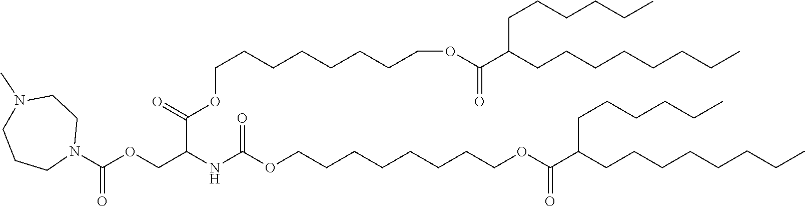 [SMILES: CCCCCCCCC(CCCCCC)C(=O)OCCCCCCCCOC(=O)NC(COC(=O)N1CCCN(C)CC1)C(=O)OCCCCCCCCOC(=O)C(CCCCCC)CCCCCCCC] |
| 1118. | 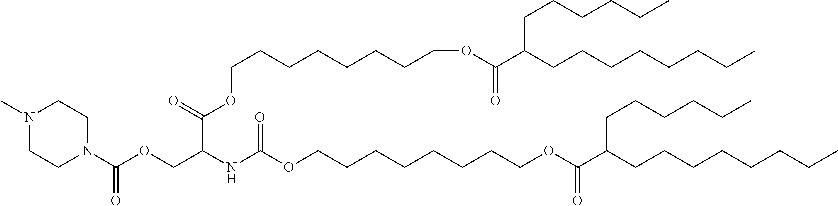 |
| 1119. | 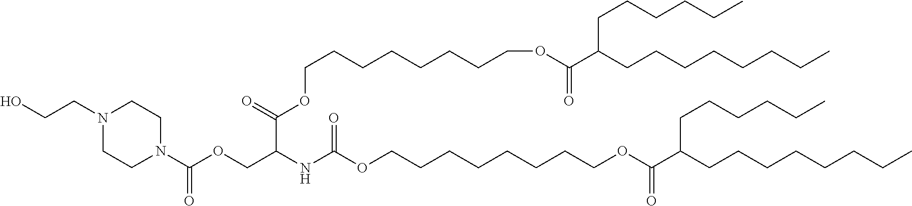 |
| 1123. | 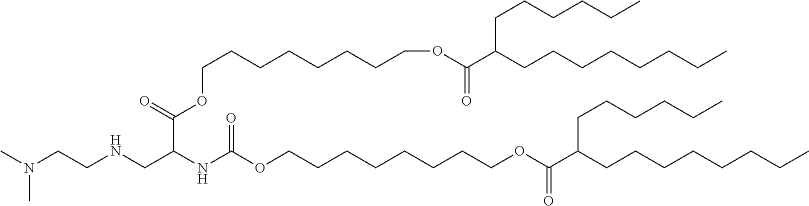 |
| 1124. | 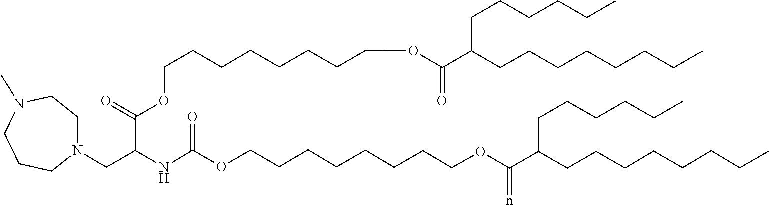 |
| 1125. | 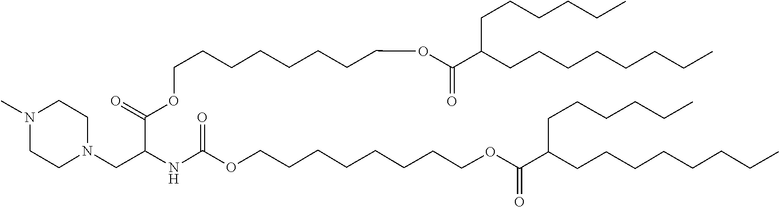 [SMILES: CCCCCCCCC(CCCCCC)C(=O)OCCCCCCCCOC(=O)NC(CN1CCN(C)CC1)C(=O)OCCCCCCCCOC(=O)C(CCCCCC)CCCCCCCC] |
| 1126. | 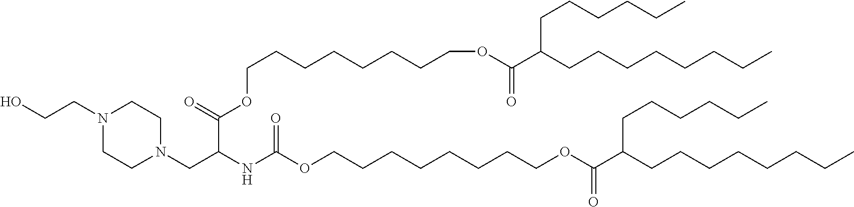 |
| 1127. | 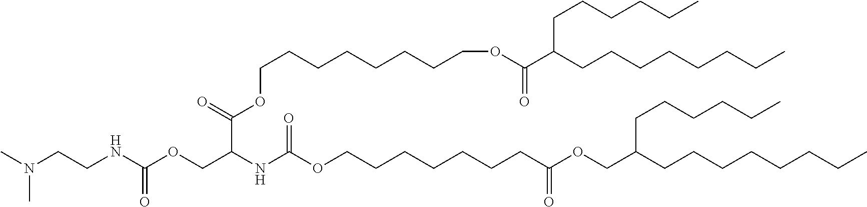 |
| 1128. | 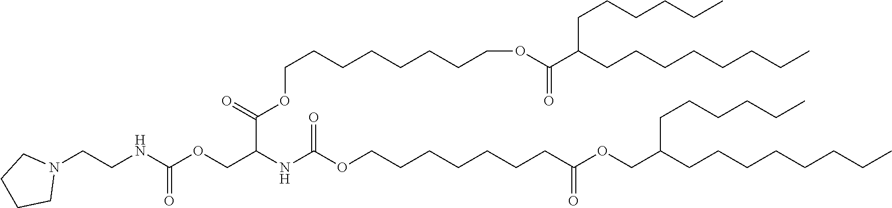 |
| 1129. | 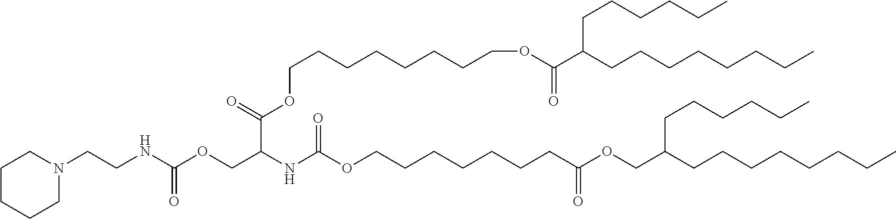 |
| 1130. | 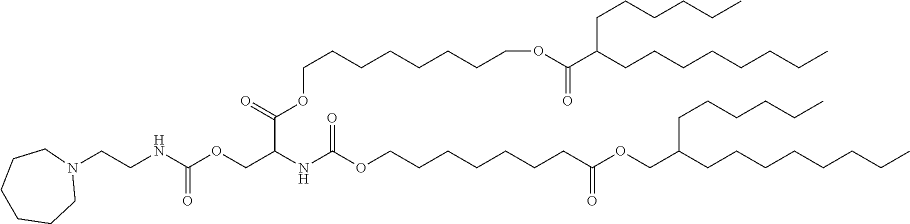 |
| 1131. | 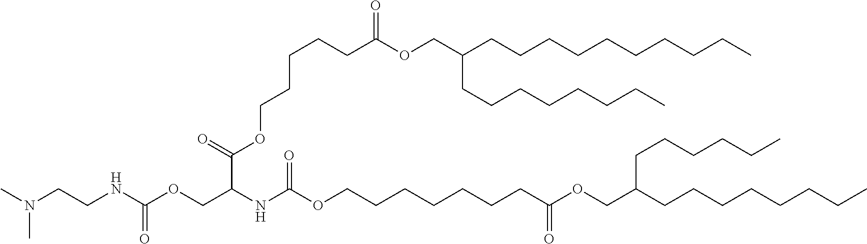 [SMILES: CCCCCCCCCCC(CCCCCCCC)COC(=O)CCCCCOC(=O)C(COC(=O)NCCN(C)C)NC(=O)OCCCCCCCC(=O)OCC(CCCCCC)CCCCCCCC] |
| 1132. | 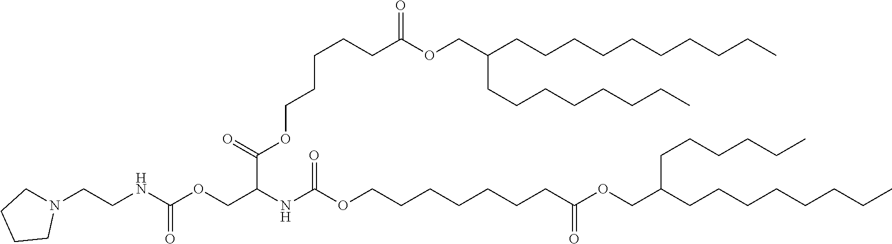 [SMILES: CCCCCCCCCCC(CCCCCCCC)COC(=O)CCCCCOC(=O)C(COC(=O)NCCN1CCCC1)NC(=O)OCCCCCCCC(=O)OCC(CCCCCC)CCCCCCCC] |
| 1133. | 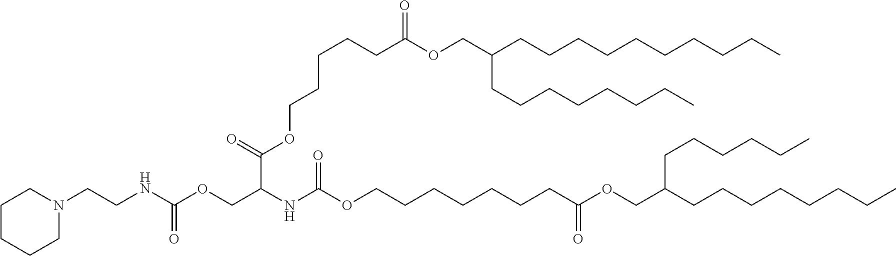 [SMILES: CCCCCCCCCCC(CCCCCCCC)COC(=O)CCCCCOC(=O)C(COC(=O)NCCN1CCCCC1)NC(=O)OCCCCCCCC(=O)OCC(CCCCCC)CCCCCCCC] |
| 1134. | 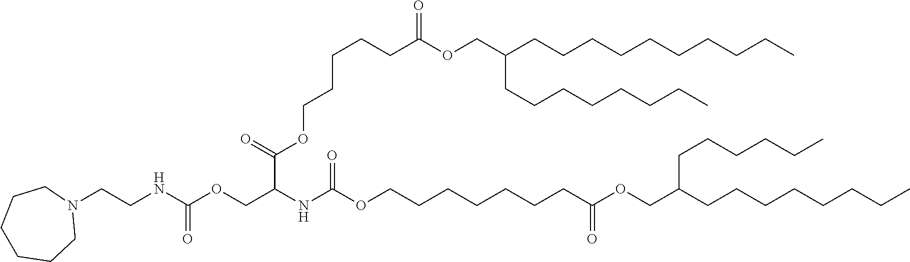 [SMILES: CCCCCCCCCCC(CCCCCCCC)COC(=O)CCCCCOC(=O)C(COC(=O)NCCN1CCCCCC1)NC(=O)OCCCCCCCC(=O)OCC(CCCCCC)CCCCCCCC] |
| 1135. | 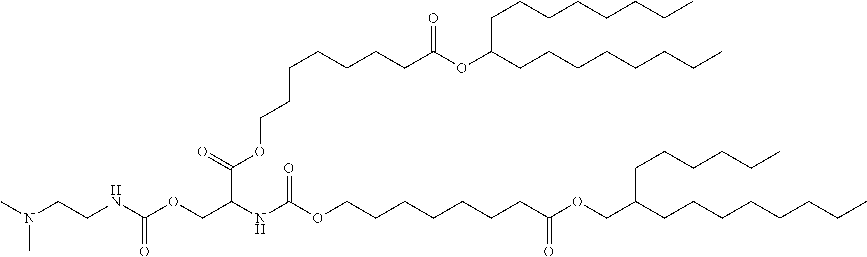 [SMILES: CCCCCCCCC(CCCCCC)COC(=O)CCCCCCCOC(=O)NC(COC(=O)NCCN(C)C)C(=O)OCCCCCCCC(=O)OC(CCCCCCCC)CCCCCCCC] |
| 1136. | 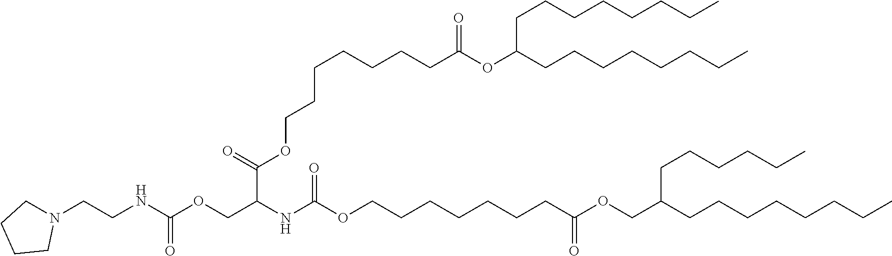 [SMILES: CCCCCCCCC(CCCCCC)COC(=O)CCCCCCCOC(=O)NC(COC(=O)NCCN1CCCC1)C(=O)OCCCCCCCC(=O)OC(CCCCCCCC)CCCCCCCC] |
| 1137. | 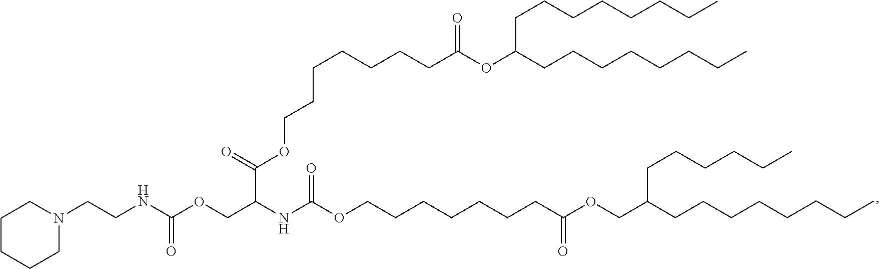 [SMILES: CCCCCCCCC(CCCCCC)COC(=O)CCCCCCCOC(=O)NC(COC(=O)NCCN1CCCCC1)C(=O)OCCCCCCCC(=O)OC(CCCCCCCC)CCCCCCCC] |
| 1138. | 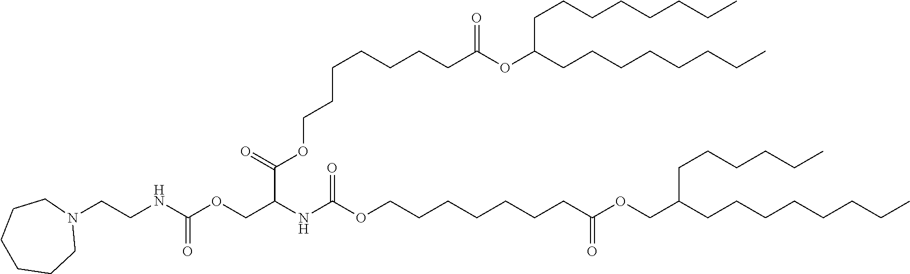 [SMILES: CCCCCCCCC(CCCCCC)COC(=O)CCCCCCCOC(=O)NC(COC(=O)NCCN1CCCCCC1)C(=O)OCCCCCCCC(=O)OC(CCCCCCCC)CCCCCCCC] |
| 1139. | 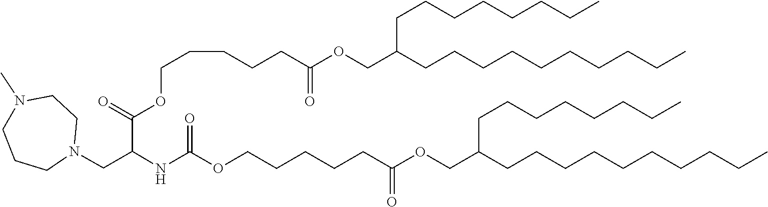 [SMILES: CCCCCCCCCCC(CCCCCCCC)COC(=O)CCCCCOC(=O)NC(CN1CCCN(C)CC1)C(=O)OCCCCCC(=O)OCC(CCCCCCCC)CCCCCCCCCC] |
| 1140. | 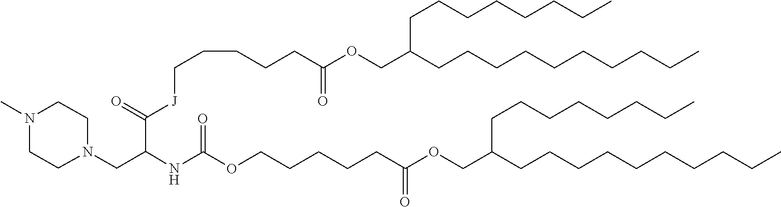 |
| 1141. | 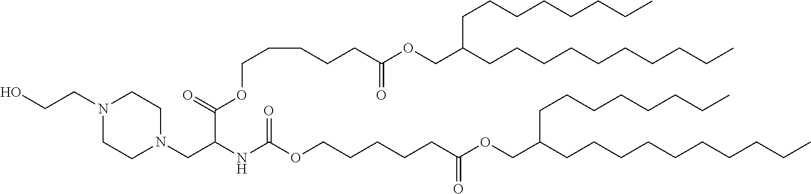 |
| 1142. | 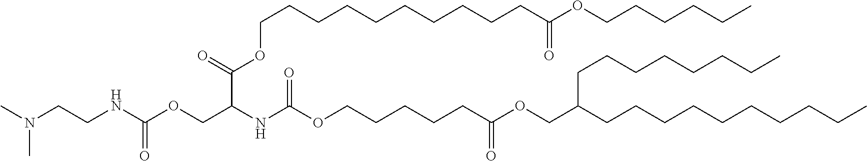 [SMILES: CCCCCCCCCCC(CCCCCCCC)COC(=O)CCCCCOC(=O)NC(COC(=O)NCCN(C)C)C(=O)OCCCCCCCCCCC(=O)OCCCCCC] |
| 1143. | 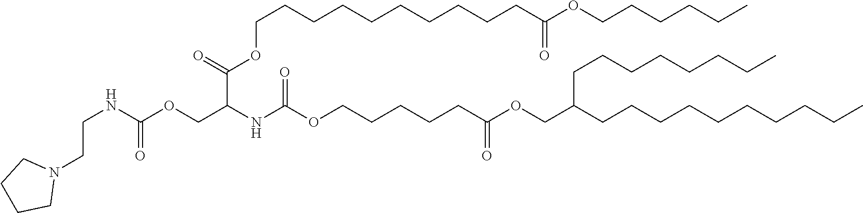 [SMILES: CCCCCCCCCCC(CCCCCCCC)COC(=O)CCCCCOC(=O)NC(COC(=O)NCCN1CCCC1)C(=O)OCCCCCCCCCCC(=O)OCCCCCC] |
| 1144. | 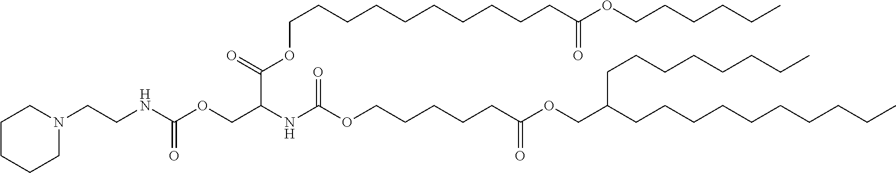 [SMILES: CCCCCCCCCCC(CCCCCCCC)COC(=O)CCCCCOC(=O)NC(COC(=O)NCCN1CCCCC1)C(=O)OCCCCCCCCCCC(=O)OCCCCCC] |
| 1145. | 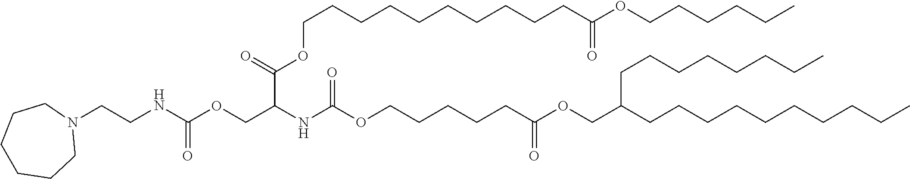 [SMILES: CCCCCCCCCCC(CCCCCCCC)COC(=O)CCCCCOC(=O)NC(COC(=O)NCCN1CCCCCC1)C(=O)OCCCCCCCCCCC(=O)OCCCCCC] |
| 1146. | 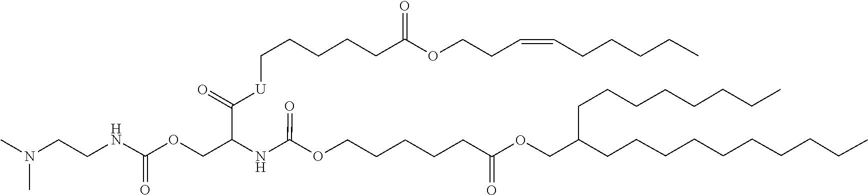 |
| 1147. | 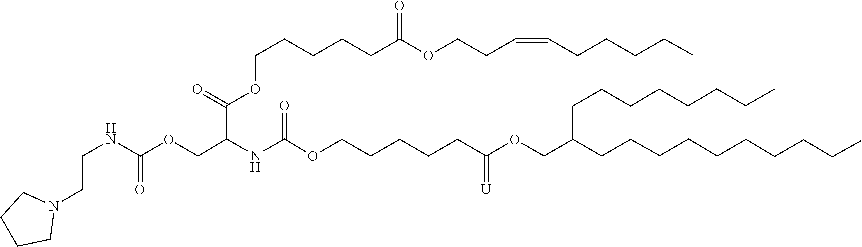 |
| 1148. | 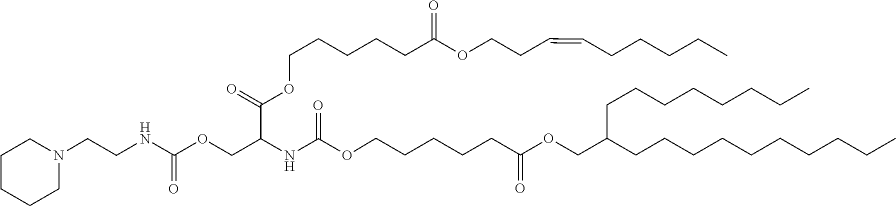 [SMILES: CCCCC/C=C\CCOC(=O)CCCCCOC(=O)C(COC(=O)NCCN1CCCCC1)NC(=O)OCCCCCC(=O)OCC(CCCCCCCC)CCCCCCCCCC] |
| 1149. | 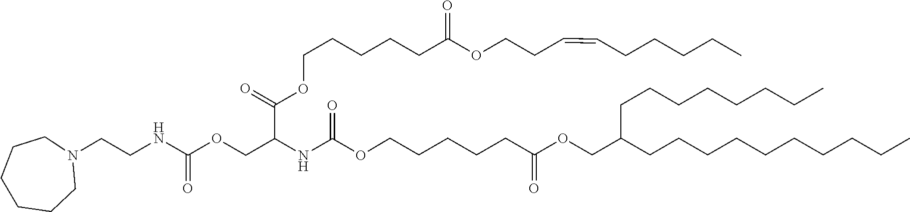 [SMILES: CCCCC/C=C\CCOC(=O)CCCCCOC(=O)C(COC(=O)NCCN1CCCCCC1)NC(=O)OCCCCCC(=O)OCC(CCCCCCCC)CCCCCCCCCC] |
| 1150. | 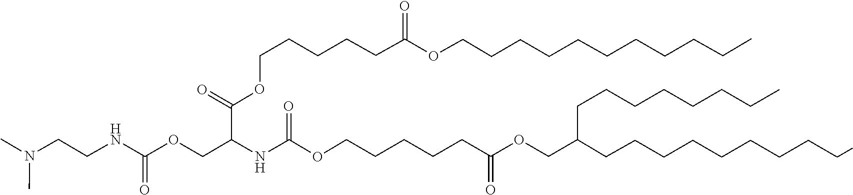 [SMILES: CCCCCCCCCCCOC(=O)CCCCCOC(=O)C(COC(=O)NCCN(C)C)NC(=O)OCCCCCC(=O)OCC(CCCCCCCC)CCCCCCCCCC] |
| 1151. | 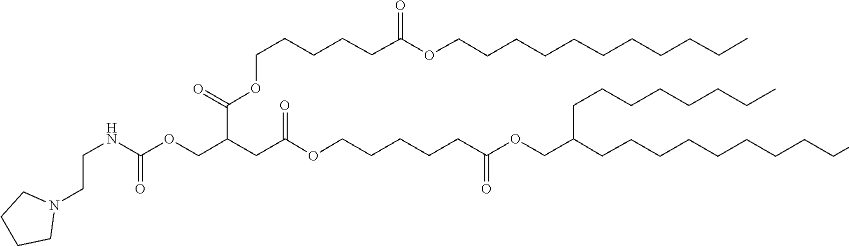 [SMILES: CCCCCCCCCCCOC(=O)CCCCCOC(=O)C(COC(=O)NCCN1CCCC1)CC(=O)OCCCCCC(=O)OCC(CCCCCCCC)CCCCCCCCCC] |
| 1152. | 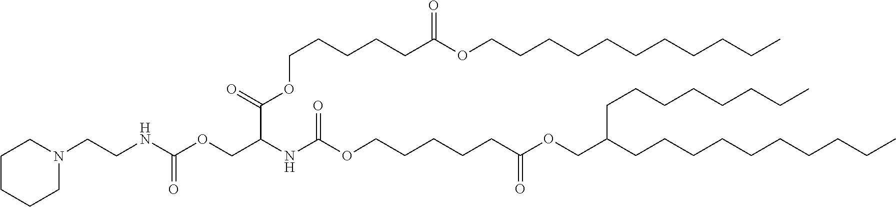 [SMILES: CCCCCCCCCCCOC(=O)CCCCCOC(=O)C(COC(=O)NCCN1CCCCC1)NC(=O)OCCCCCC(=O)OCC(CCCCCCCC)CCCCCCCCCC] |
| 1153. | 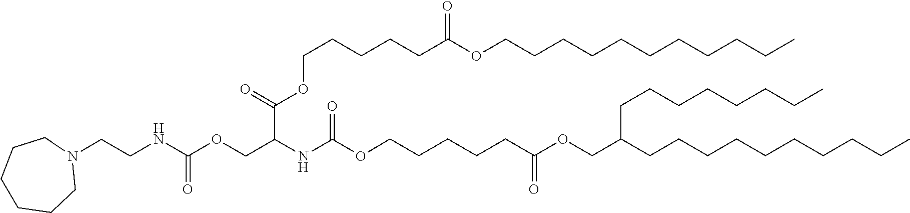 [SMILES: CCCCCCCCCCCOC(=O)CCCCCOC(=O)C(COC(=O)NCCN1CCCCCC1)NC(=O)OCCCCCC(=O)OCC(CCCCCCCC)CCCCCCCCCC] |
| 1154. | 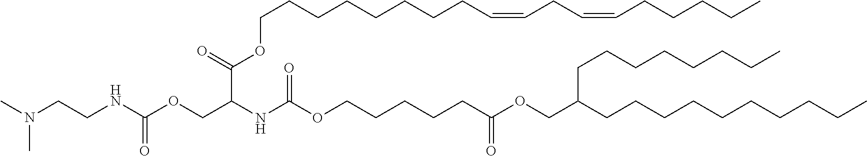 [SMILES: CCCCC/C=C\C/C=C\CCCCCCCCOC(=O)C(COC(=O)NCCN(C)C)NC(=O)OCCCCCC(=O)OCC(CCCCCCCC)CCCCCCCCCC] |
| 1155. | 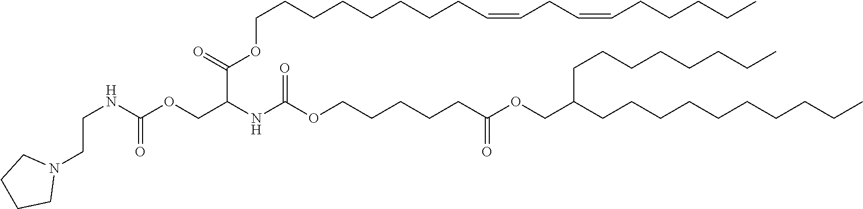 [SMILES: CCCCC/C=C\C/C=C\CCCCCCCCOC(=O)C(COC(=O)NCCN1CCCC1)NC(=O)OCCCCCC(=O)OCC(CCCCCCCC)CCCCCCCCCC] |
| 1156. | 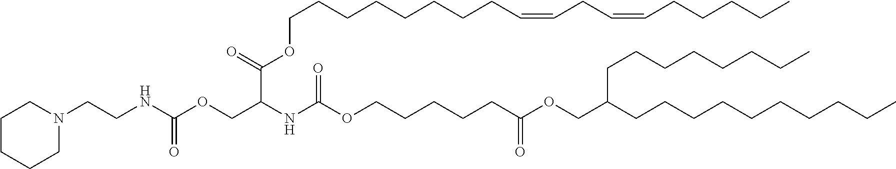 [SMILES: CCCCC/C=C\C/C=C\CCCCCCCCOC(=O)C(COC(=O)NCCN1CCCCC1)NC(=O)OCCCCCC(=O)OCC(CCCCCCCC)CCCCCCCCCC] |
| 1157. | 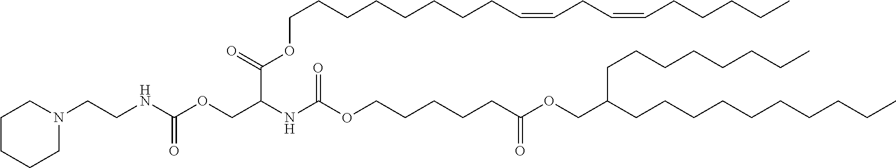 [SMILES: CCCCC/C=C\C/C=C\CCCCCCCCOC(=O)C(COC(=O)NCCN1CCCCC1)NC(=O)OCCCCCC(=O)OCC(CCCCCCCC)CCCCCCCCCC] |
| 1165. | 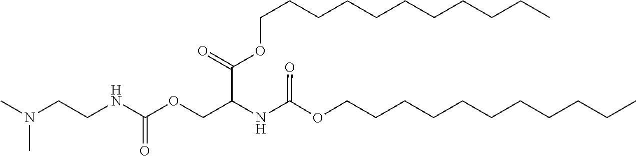 [SMILES: CCCCCCCCCCCOC(=O)NC(COC(=O)NCCN(C)C)C(=O)OCCCCCCCCCCC] |
| 1166. | 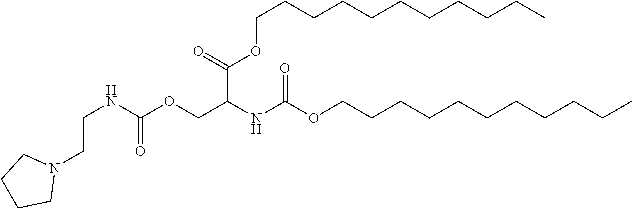 [SMILES: CCCCCCCCCCCOC(=O)NC(COC(=O)NCCN1CCCC1)C(=O)OCCCCCCCCCCC] |
| 1167. | 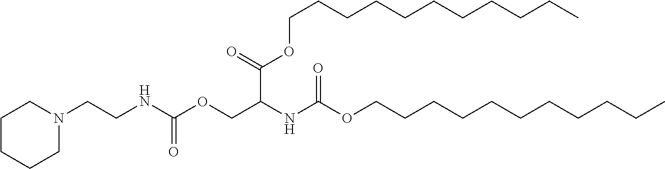 [SMILES: CCCCCCCCCCCOC(=O)NC(COC(=O)NCCN1CCCCC1)C(=O)OCCCCCCCCCCC] |
| 1168. | 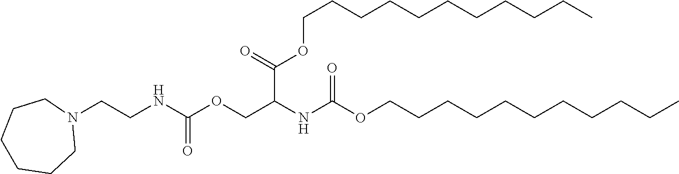 [SMILES: CCCCCCCCCCCOC(=O)NC(COC(=O)NCCN1CCCCCC1)C(=O)OCCCCCCCCCCC] |
| 1169. | 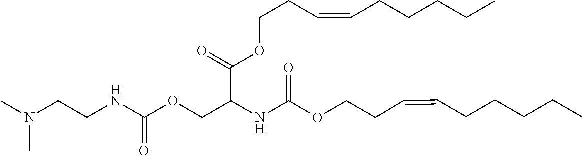 [SMILES: CCCCC/C=C\CCOC(=O)NC(COC(=O)NCCN(C)C)C(=O)OCC/C=C\CCCCC] |
| 1170. | 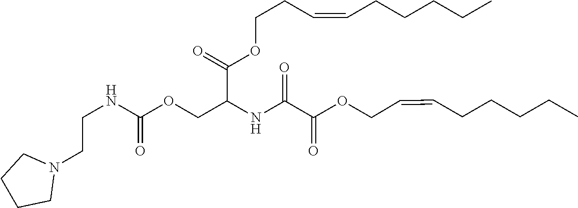 [SMILES: CCCCC/C=C\CCOC(=O)C(COC(=O)NCCN1CCCC1)NC(=O)C(=O)OC/C=C\CCCCC] |
| 1171. | 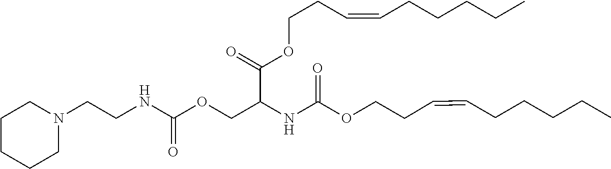 [SMILES: CCCCC/C=C\CCOC(=O)NC(COC(=O)NCCN1CCCCC1)C(=O)OCC/C=C\CCCCC] |
| 1172. | 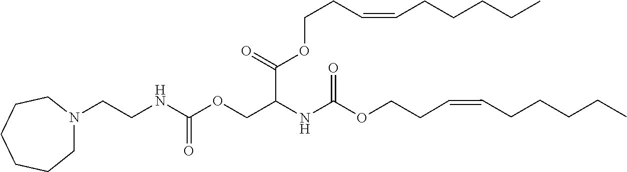 [SMILES: CCCCC/C=C\CCOC(=O)NC(COC(=O)NCCN1CCCCCC1)C(=O)OCC/C=C\CCCCC] |

In some aspects, the present invention provides an intermediate compound represented by the structure of formula (VI):

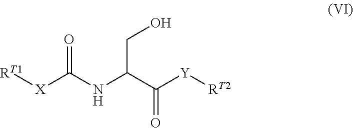

wherein
each of the groups is as defined herein.

In some aspects, the present invention provides a method of preparing the cationic lipid of the present invention, comprising a step of reacting a compound of formula (VI) with a compound of formula (VII), to yield the cationic lipid of formula (II):

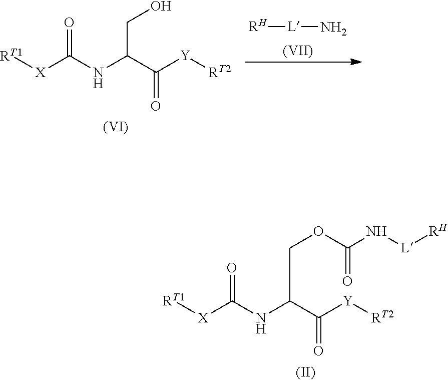

wherein each of the groups is as defined herein; and
the reaction is conducted in the presence of CDI.

In some aspects, the present invention provides a method of preparing the cationic lipid of the present invention, comprising a step of reacting a compound of formula (VI) with a compound of formula (VII), to yield the cationic lipid of formula (III):

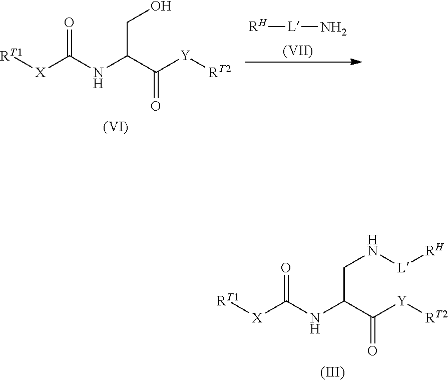

wherein each of the groups is as defined herein; and
the reaction is conducted in the presence of TsCl.

In some aspects, the present invention provides a nanoparticle composition, comprising a cationic lipid of the invention.

In some aspects, the present invention provides a nanoparticle composition, further comprising one or more selected from the group of a phospholipid, a PEG lipid and a structural lipid. In some aspects, the present invention provides a nanoparticle composition, further comprising a phospholipid, a PEG lipid and a structural lipid.

In some aspects, the present invention provides a pharmaceutical composition comprising a nanoparticle composition according to the present invention and a pharmaceutically acceptable carrier.

As used herein, “a therapeutic and/or prophylactic nucleic acid molecule” refers to a nucleic acid molecule encoding a therapeutic and/or prophylactic protein which can be translated in cells. Such a nucleic acid molecule can be any suitable forms of nucleic acid molecules, e.g. any linear RNA or any circular RNA.

In some aspects, the present invention provides a method of delivering a nucleic acid molecule to a cell, including the step of administering to a subject (i) the nanoparticle composition of the invention and (ii) said nucleic acid molecule, in which administering involves contacting the cell with the nanoparticle composition, whereby the nucleic acid molecule is delivered to the cell. In some aspects, the present invention provides a method of delivering a nucleic acid molecule to a cell, including the step of administering to a subject a composition including (i) the nanoparticle composition of the invention and (ii) said nucleic acid molecule, in which administering involves contacting the cell with the nanoparticle composition, whereby the nucleic acid molecule is delivered to the cell. In embodiments, said nucleic molecule is a protein-coding nucleic acid molecule. In embodiments, said nucleic molecule is a therapeutic and/or prophylactic nucleic acid molecule. In embodiments, the protein-coding molecule can be translated in vivo. n embodiments, the nucleic acid molecule is encapsulated in the nanoparticle composition of the present invention. In embodiments, the nucleic acid molecule is attached to the nanoparticle composition of the present invention. As used herein “attached to” refers to attaching the nucleic acid to the nanoparticle composition of the present invention via any physical or chemical means.

In some aspects, the present invention provides a method of delivering a therapeutic and/or prophylactic nucleic acid molecule to a cell, including the step of administering to a subject a pharmaceutical composition comprising (i) the nanoparticle composition of the invention and (ii) a therapeutic and/or prophylactic nucleic acid molecule, in which administering involves contacting the cell with the nanoparticle composition, whereby the therapeutic and/or prophylactic nucleic acid molecule is delivered to the cell. In embodiments, the nucleic acid molecule is encapsulated in the nanoparticle composition of the present invention. In embodiments, the nucleic acid molecule is attached to the nanoparticle composition of the present invention. As used herein “attached to” refers to attaching the nucleic acid to the nanoparticle composition of the present invention via any physical or chemical means.

In embodiments, the subject is a mammal. In embodiments, the mammal is a human.

In some aspects, the present invention provides a method of delivering a protein-coding nucleic acid molecule to a cell, including the step of administering to a subject (i) the nanoparticle composition of the invention and (ii) a protein-coding nucleic acid molecule, in which administering involves contacting the cell with the nanoparticle composition of the present invention, whereby the nucleic acid is delivered to the cell. In embodiments, the protein-coding nucleic acid molecule is encapsulated in the nanoparticle composition of the present invention. In embodiments, the protein-coding nucleic acid molecule is attached to the nanoparticle composition of the present invention. As used herein “attached to” refers to attaching the nucleic acid to the nanoparticle composition of the present invention via any physical or chemical means.

In some aspects, the present invention provides a method of providing a polypeptide of interest in a cell, including the step of contacting the cell with a nanoparticle composition of the invention and (ii) a nucleic acid molecule encoding the polypeptide of interest, whereby the nucleic acid molecule is capable of being translated in the cell to produce the polypeptide. In embodiments, the nucleic acid molecule is an mRNA molecule, a siRNA molecule, or a circular RNA molecule. In embodiments, the nucleic acid molecule is a linear RNA. In embodiments, the nucleic acid molecule is a circular RNA.

In some aspects, the present invention provides a nanoparticle composition for use in the manufacture of a medicament for the treatment of a disease or disorder in a subject or mammal in need thereof, wherein the nanoparticle composition includes (i) a lipid component including a phospholipid, a PEG lipid, a structural lipid, and a cationic lipid of the invention and (ii) a therapeutic and/or prophylactic nucleic acid molecule. In embodiments, the nucleic acid molecule is an mRNA, a siRNA or a circular RNA. In embodiments, the nucleic acid molecule is a linear RNA. In embodiments, the nucleic acid molecule is a circular RNA.

In some aspects, the present invention provides a nanoparticle composition for use in the manufacture of a medicament for the treatment of a disease or disorder in a subject or mammal in need thereof, wherein the nanoparticle composition includes (i) a cationic lipid of the invention and (ii) a nucleic acid molecule encoding a therapeutic and/or prophylactic protein. In embodiments, the nucleic acid molecule is an mRNA, a siRNA or a circular RNA. In embodiments, the nucleic acid molecule is a linear RNA. In embodiments, the nucleic acid molecule is a circular RNA.

In embodiments of the present invention, the subject is a mammal.

In embodiments of the present invention, the mammal is a human.

In some aspects, the present invention provides use of the cationic lipid of the invention, for the manufacture of a nanoparticle composition.

In some aspects, the present invention provides use of the cationic lipid of the invention or use of the nanoparticle composition of the invention for the manufacture of a medicament for the treatment of a disease or disorder in a subject in need thereof.

In embodiments, the subject is a mammal.

In embodiments, the mammal is a human.

In some aspects, the present invention provides a method of synthesizing a cationic lipid of Formula (I)-1, (I)-1-A, (I)-1-B, (I)-1-C, (I), (IA), (II), (III), (IV), or (V) and methods of making a nanoparticle composition including a lipid component comprising the cationic lipid of Formula (I)-1, (I)-1-A, (I)-1-B, (I)-1-C, (I), (IA), (II), (III), (IV), or (V).

### EXAMPLES

The following examples are included to further illustrate the invention described herein and to demonstrate embodiments of the invention. It should be appreciated by those of skill in the art that the techniques disclosed in the examples which follow represent techniques discovered by the inventors to function well in the practice of the invention, and thus can be considered to constitute modes for its practice. However, those of skill in the art should, in light of the present disclosure, appreciate that many changes can be made in the specific embodiments which are disclosed and still obtain a like or similar result which are within the spirit and scope of the invention.

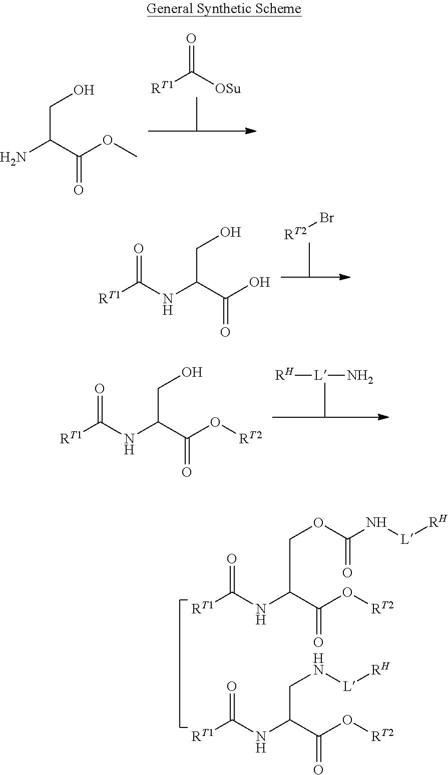

The scheme above depicts the general synthetic scheme of the target lipids, wherein each group is as defined herein. Initially a Serine protected with a methyl ester is reacted with a fatty acid which is initially activated with 3-(((ethylimino)methylene)amino)-N,N-dimethylpropan-1-amine (EDC) and N-Hydroxysuccinimide (NHS). The NHS ester is then reacted with the Serine in a THF water mixture with sodium bicarbonate. Sodium hydroxide is then added to cleave the methyl ester. The free acid serine is then reacted with a long chain alkyl halide in DMF with potassium carbonate as base. The resulting alcohol is then either converted to a carbamate by activation with carbonyldiimidazole (CDI) and further reacted with an amine, or it is converted to a secondary amine by activation Tosyl chloride and a sequential reaction with an amine.

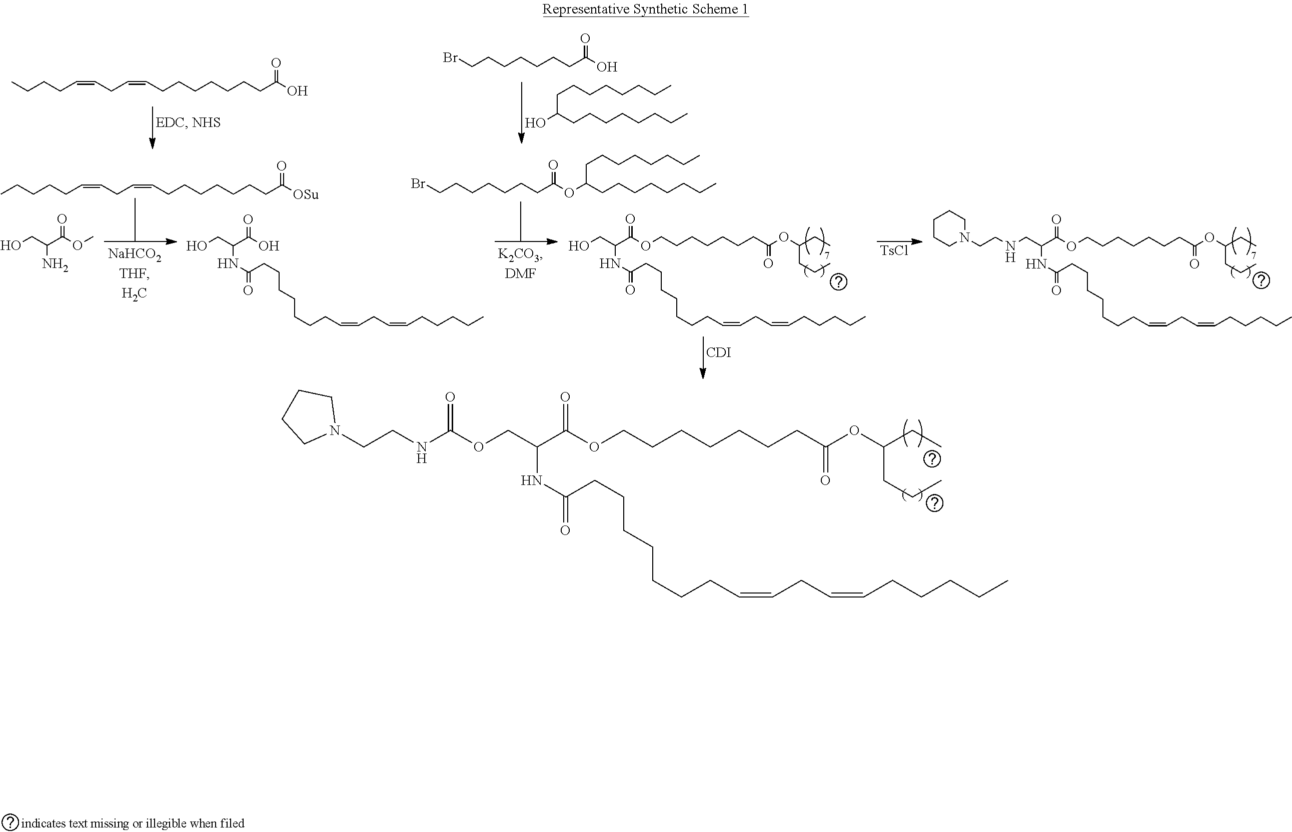

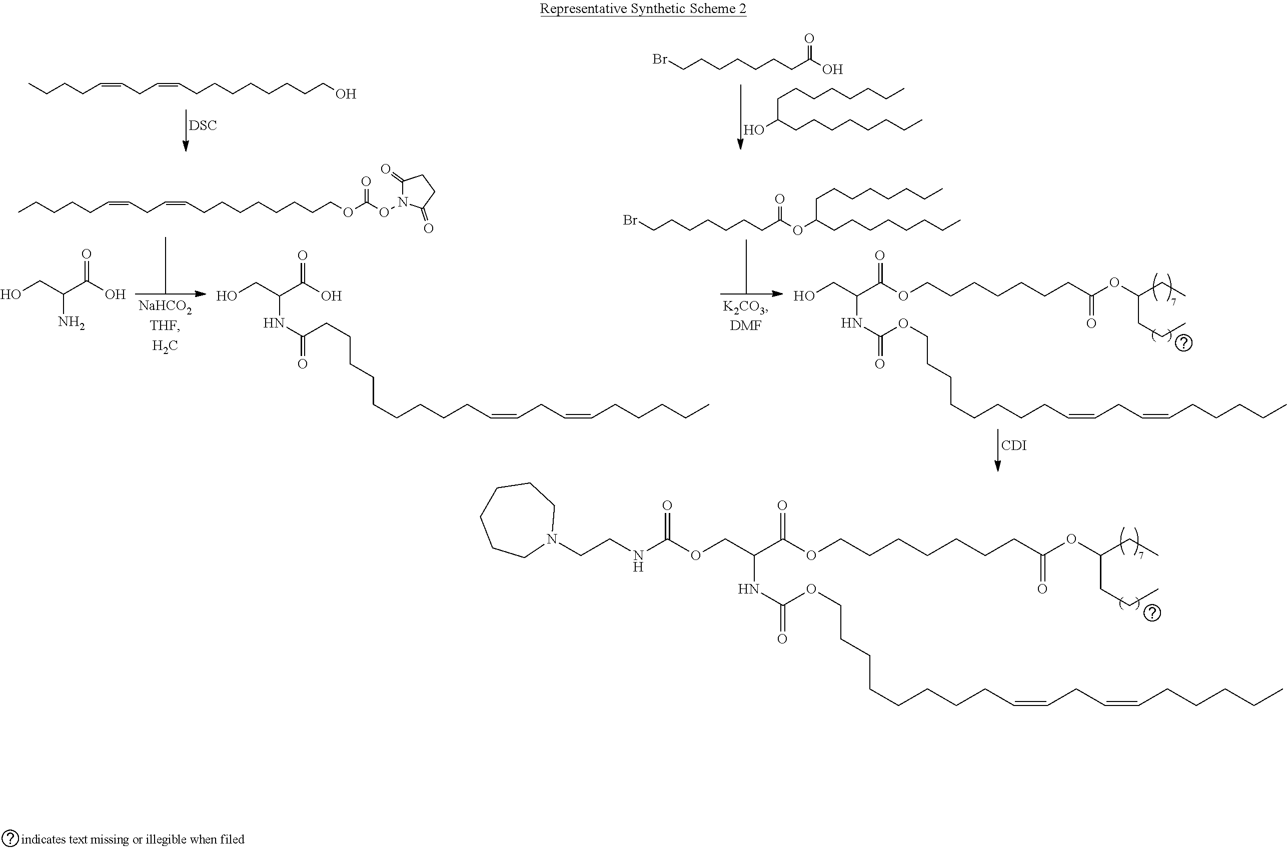

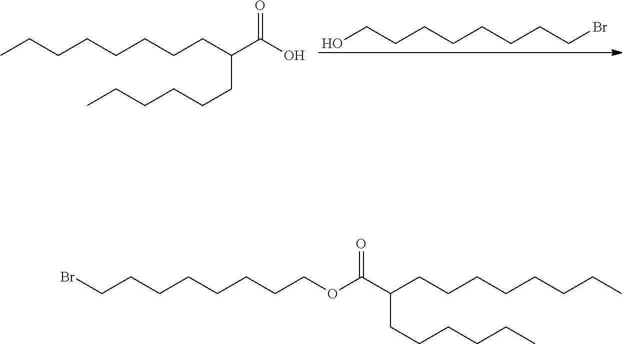 [SMILES: CCCCCCCCC(CCCCCC)C(=O)O ; OCCCCCCCCBr ; CCCCCCCCC(CCCCCC)C(=O)OCCCCCCCCBr]

8-bromooctyl 2-hexyldecanoate 2-hexyldecanoic acid (5 g, 19.50 mmol), 8-bromooctan-1-ol (4.08 g, 19.50 mmol) and TsOH (0.185 g, 0.975 mmol) were dissolved in 80 mL Toluene dry. The reaction was left to stir at 125° C. O/N under Ar. The solvent was removed by evaporation and the crude material was purifed by column chromatography (EtOAc 10% in Hexane) to afford the compound as a colorless oil (6.55 gr; 75%). ¹H NMR (400 MHz, CDCl₃): δ 0.87 (6H, t, J=6.61 Hz), 1.25 (29H, m), 1.60 (5H, m), 1.85 (2H, quint, J=6.92 Hz), 2.31 (1H, m), 3.40 (2H, t, J=6.83 Hz), 4.06 (2H, t, J=6.60 Hz). MS [ESI]: m/z: [M+H] calc. 447.5 obs. 447.5

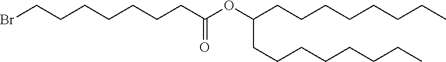 [SMILES: CCCCCCCCC(CCCCCCCC)OC(=O)CCCCCCCBr]

heptadecan-9-yl 8-bromooctanoate was synthesized according to method 1 from 8-bromooctanoic acid and heptadecan-9-ol to afford the compound as a yellow oil. ¹H NMR (400 MHz, CDCl3): δ 0.90 (6H, t, J=6.77 Hz), 1.28 (34H, m), 1.64 (2H, m), 1.86 (2H, quint, J=6.92 Hz), 2.30 (2H, t, J=7.44 Hz), 3.41 (2H, t, J=6.83 Hz), 4.89 (1H, quint, J=6.24 Hz).

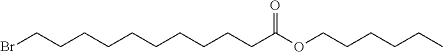 [SMILES: CCCCCCOC(=O)CCCCCCCCCCBr]

hexyl 11-bromoundecanoate was synthesized according to method 1 from 11-bromoundecanoic acid and hexan-1-ol to afford the compound as a colorless oil. ¹H NMR (400 MHz, CDCl₃): δ 0.89 (3H, t, J=6.86 Hz), 1.28 (18H, m), 1.59 (5H, m), 1.76 (1H, t, J=7.35 Hz), 2.28 (2H, t, J=7.51 Hz), 3.40 (1H, t, J=6.86 Hz), 3.52 (1H, t, J=6.75 Hz), 4.05 (2H, t, J 6.72 Hz).

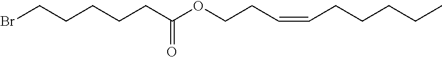 [SMILES: CCCCC/C=C\CCOC(=O)CCCCCBr]

(Z)-non-3-en-1-yl 6-bromohexanoate was synthesized according to method 1 from 6-bromohexanoic acid and (Z)-non-3-en-1-ol to afford the title compound as a colorless oil. ¹H NMR (400 MHz, CDCl₃): δ 0.90 (3H, t, J=6.79 Hz), 1.32 (6H, m), 1.49 (2H, m), 1.68 (2H, m), 1.89 (2H, quint, J=6.89 Hz), 2.05 (2H, q, J=7.34 Hz), 2.30-2.44 (4H, 2.33 (t, J=7.42 Hz), 2.39 (q, J=7.07 Hz)), 3.42 (2H, t, J=6.76 Hz), 4.09 (2H, t, J=6.92 Hz), 5.36 (1H, m), 5.52 (1H, m).

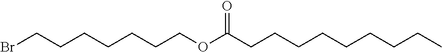 [SMILES: CCCCCCCCCC(=O)OCCCCCCCBr]

7-bromoheptyl decanoate was synthesized according to method 1 from decanoic acid and 7-bromoheptan-1-ol to afford the title compound as a colorless oil. ¹H NMR (400 MHz, CDCl₃): δ 0.87 (3H, t, J=6.80 Hz), 1.20-1.48 (18H, 1.21 (m), 1.26 (m), 1.35 (m)), 1.62 (4H, m), 1.80-1.91 (2H, 1.85 (t, J=7.34 Hz), 1.88 (s)), 2.28 (2H, t, J=7.38 Hz), 3.39 (2H, t, J=6.66 Hz), 4.05 (2H, t, J=6.46 Hz).

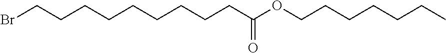 [SMILES: CCCCCCCOC(=O)CCCCCCCCCBr]

heptyl 10-bromodecanoate was synthesized according to method 1 from heptan-1-ol and 10-bromodecanoic acid to afford title compound as a colorless oil. ¹H NMR (400 MHz, CDCl3): δ 0.87-0.93 (311, 0.90 (t, J=6.93 Hz), 0.91 (s)), 1.31 (18H, m), 1.63 (4H, quint, J=6.97 Hz), 1.86 (2H, quint, J=6.93 Hz), 2.30 (2H, t, J=7.51 Hz), 3.41 (2H, t, J=6.86 Hz), 4.07 (2H, t, J=6.73 Hz).

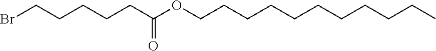 [SMILES: CCCCCCCCCCCOC(=O)CCCCCBr]

undecyl 6-bromohexanoate was synthesized according to method 1 from undecan-1-ol and 6-bromohexanoic acid to afford the title compound as a colorless oil. ¹H NMR (400 MHz, CDCl₃): δ 0.90 (3H, t, J=6.75 Hz), 1.28 (16H, m), 1.49 (2H, m), 1.58-1.73 (4H, m), 1.89 (2H, quint, J=6.89 Hz), 2.33 (2H, t, J=7.42 Hz), 3.42 (2H, t, J=6.76 Hz), 4.08 (2H, t, J=6.74 Hz).

### Intermediate Example 2 (Method 2)

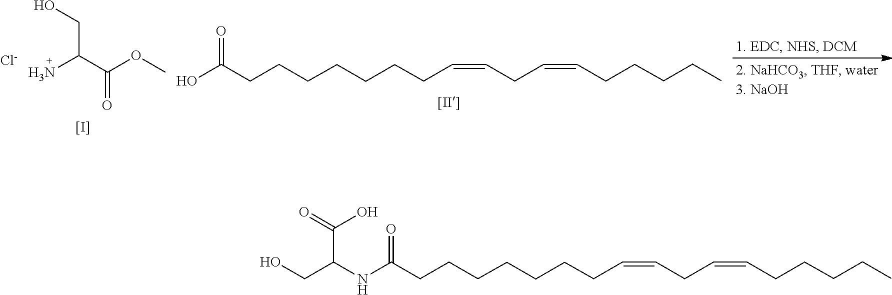

(9Z,12Z)-octadeca-9,12-dienoic acid (2.5 g, 8.91 mmol) and 1-hydroxypyrrolidine-2,5-dione (2.56 g, 22.29 mmol) dissolved in 35 mL DCM and the solution was cooled to 0° C. EDC (2.56 g, 13.37 mmol) was added and the reaction was stirred for 30 mins at 0° C., it was then allowed to heat to RT and was allowed to stir until the full conversion of the starting material. 20 mL DCM were added and the reaction was washed with water (30 mL×2) The organic phase was dried over MgSO₄, filtered, evaporated and redissolved in 30 mL THF. 3-hydroxy-1-methoxy-1-oxopropan-2-aminium chloride (1.387 g, 8.91 mmol) and sodium bicarbonate (2.247 g, 26.7 mmol) were dissolved in 10 mL water and the THF solution was added. The solution was heated to 75° C. and stirred until the full consumption of the activated acid. Sodium hydroxide (3.57 g, 89 mmol) was dissolved in 5 mL water and the solution was added to the reaction mixture. The solution was left to stir at RT for 20 mins. HCl 32% was added to adjust pH=1. THE was removed by evaporation. The aqueous phase was extracted with DCM (35 mL×2), the combined organic phases were dried over MgSO₄, filtered and evaporated to acquire the title compound as a white amorphous solid which was used in the next reaction without further purification. MS [ESI]: m/z: [M+H] calc. 368.5 obs. 369.4

### Intermediate Example 3 (Method 3)

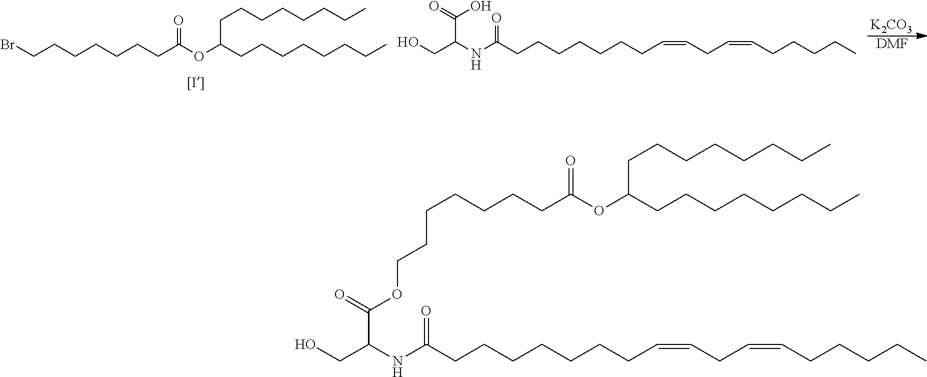

((9Z,12Z)-octadeca-9,12-dienoyl)serine (3 g, 8.16 mmol) and heptadecan-9-yl 8-bromooctanoate (3.77 g, 8.16 mmol) were dissolved in 30 mL DMF, potassium carbonate (1.128 g, 8.16 mmol) was added and the reaction was left to stir at 75° C. until the full consumption of the starting materials (2 hrs). 100 mL of brine were added, and the aqueous phase was extracted with hexane (20 mL×2) the combined organic phases were dried over MgSO₄ and filtered. 3 gr of silica were added and the solvent was removed under vacuum. Chromatography yielded the title compound as a colorless oil (987.1 mg; 22% yield).

MS [ESI]: m/z: [M+H] calc. 748.2 obs. 748.8

### Intermediate Example 4 (Method 4)

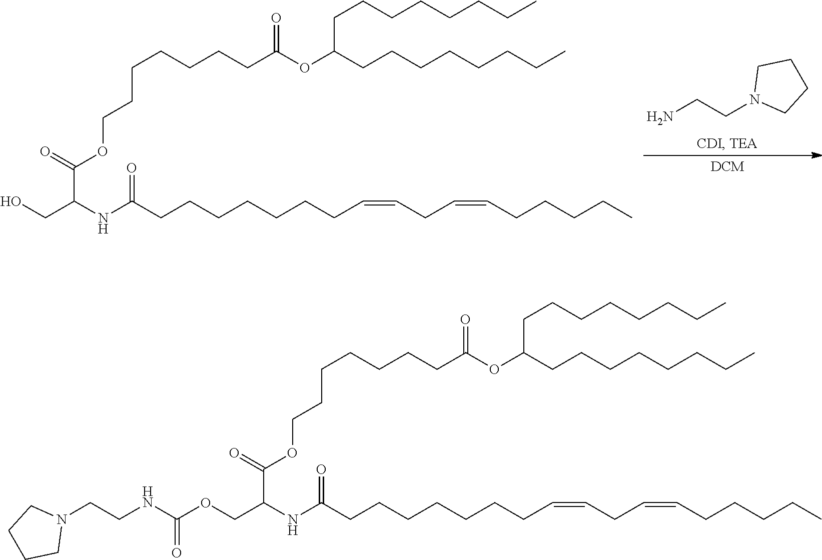 [SMILES: CCCCC/C=C\C/C=C\CCCCCCCC(=O)NC(CO)C(=O)OCCCCCCCC(=O)OC(CCCCCCCC)CCCCCCCC ; NCCN1CCCC1 ; CCCCC/C=C\C/C=C\CCCCCCCC(=O)NC(COC(=O)NCCN1CCCC1)C(=O)OCCCCCCCC(=O)OC(CCCCCCCC)CCCCCCCC]

heptadecan-9-yl 8-((((9Z,12Z)-octadeca-9,12-dienoyl)seryl)oxy)octanoate 450 mg, 0.601 mmol was dissolved in 4 mL DCM. CDI (127 mg, 0.782 mmol) was then added and the solution was allowed to stir for 1 hr and TEA (184 μl, 1.323 mmol) was then added followed by 2-(pyrrolidin-1-yl)ethan-1-amine (150.1 mg, 1.323 mmol). The reaction was left to stir at RT until full consumption of the starting material. 20 mL of water were added to the reaction which was allowed to stir for 5 min. The contents of the vessel were transferred to a separatory funnel and 30 mL DCM were added. The phases were separated, dried over MgSO₄ and filtered. Chromatography yielded the title compound as a colorless oil with a yellow tint (208 mg; 37.9% yield). ¹H NMR (400 MHz, CDCl₃): δ 0.88 (9H, m), 1.26 (45H, s), 1.48-1.51 (3H, m), 1.59-1.65 (8H, m), 1.76-1.80 (3H, m), 2.02-2.07 (4H, m), 2.28 (4H, m), 2.48-2.51 (311, m), 2.77 (2H, t, J=6.58 Hz), 4.15 (2H, t, J=6.70 Hz), 5.35 (4H, s), 4.83-4.89 (2H, m), 4.78-4.81 (1H, m), 4.41-4.45 (1H, m), 4.31-4.36 (1H, m), 6.40 (1H, br s), 2.55-2.59 (2H, m), 3.25-3.29 (2H, m). MS [ESI]: m/z: [M+H]. calc. 889.4 obs. 889.9

### Intermediate Example 5 (Method 5)

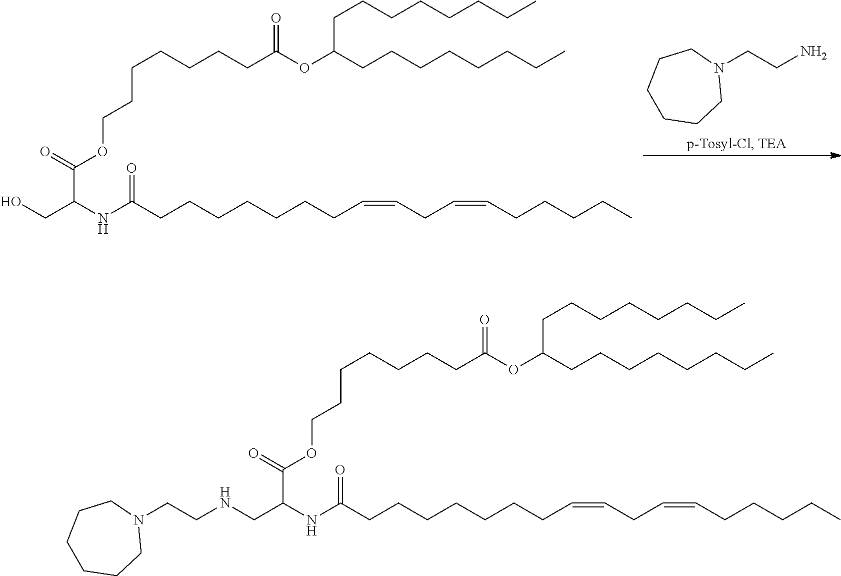 [SMILES: CCCCC/C=C\C/C=C\CCCCCCCC(=O)NC(CO)C(=O)OCCCCCCCC(=O)OC(CCCCCCCC)CCCCCCCC ; NCCN1CCCCCC1 ; CCCCC/C=C\C/C=C\CCCCCCCC(=O)NC(CNCCN1CCCCCC1)C(=O)OCCCCCCCC(=O)OC(CCCCCCCC)CCCCCCCC]

heptadecan-9-yl 8-((((9Z,12Z)-octadeca-9,12-dienoyl)seryl)oxy)octanoate (100 mg, 0.134 mmol), TEA (18.63 μl, 0.134 mmol) and p-tosyl-Cl (25.5 mg, 0.134 mmol) were dissolved in 1 mL DCM and the reaction was left to stir at RT until full consumption of the starting material. 2-(azepan-1-yl)ethan-1-amine (189 mg, 1.337 mmol) was added and the reaction was left to stir until the consumption of the intermediate. 1 mL of water were added and stirring was continued for 1 min. The contents of the vial were transferred to a separatory funnel and 20 mL DCM were added followed by 10 mL of water. The phases were separated and the aqueous phase was washed with 20 mL DCM. The organic phase was dried over MgSO₄ filtered and evaporated. Chromatography yielded the title compound as a colorless oil. (40 mg; 36% yield). ¹H NMR (500 MHz, CDCl₃): δ 0.88 (9H, m), 1.26 (45H, s), 1.44-1.65 (21H, m), 2.28 (5H, m), 2.02-2.07 (4H, m), 2.14-2.18 (2H, m), 2.65-2.69 (1H, m), 2.77 (2H, t, J=6.50 Hz), 2.93-2.97 (2H, m), 3.01-3.05 (1H, m), 4.13 (2H, t, J=6.82 Hz), 4.60-4.64 (1H, m), 4.86 (2H, i, J=6.42 Hz), 5.31-5.38 (4H, m). MS [ESI]: m/z: [M+H]. calc. 873.4 obs. 872.9

### Intermediate Example 6 (Method 6)

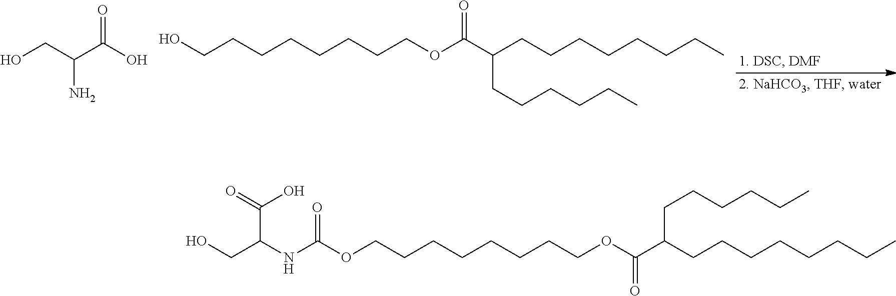 [SMILES: NC(CO)C(=O)O ; CCCCCCCCC(CCCCCC)C(=O)OCCCCCCCCO ; CCCCCCCCC(CCCCCC)C(=O)OCCCCCCCCOC(=O)NC(CO)C(=O)O]

8-hydroxyoctyl 2-hexyldecanoate (7 g, 18.20 mmol) was dissolved in 90 mL dry DMF. Bis(2,5-dioxopyrrolidin-1-yl) carbonate (9.32 g, 36.4 mmol) was added to form a solution and left to stir at RT under argon until full consumption of the starting material. Then 90 mL of water was added and extraction performed with hexane (100 mL×2). The organic phase was dried with MgSO₄, filtered, evaporated and redissolved in 100 mL THF. Serine (2.87 g, 27.3 mmol) and NaHCO₃ (4.13 g, 49.1 mmol) were dissolved in 100 mL water. The THF and water solutions were mixed and then stirred at RT until the full consumption of the starting material. THF was then removed by evaporation. EA (100 mL) and saturated NaHCO_{3(aq)} (50 mL) were added and extraction performed. The aqueous phase was adjusted to pH 1 using HCl 37% and then extracted with DCM (100 mL×3). The combined organic phases were dried with MgSO₄, filtered and evaporated to acquire the title compound as a colorless oil which was used in the next reaction without further purification (7.407 g, 78% yield). MS [ESI]: m/z: [M+H] calc. 516.4 obs. 516.5.

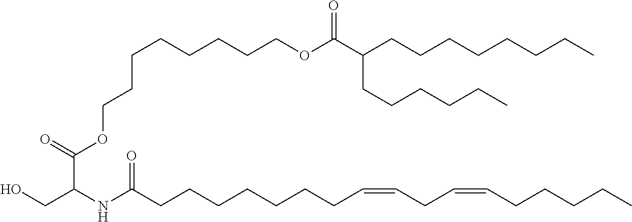 [SMILES: CCCCC/C=C\C/C=C\CCCCCCCC(=O)NC(CO)C(=O)OCCCCCCCCOC(=O)C(CCCCCC)CCCCCCCC]

8-((((9Z,12Z)-octadeca-9,12-dienoyl)seryl)oxy)octyl 2-hexyldecanoate was synthesized according to method 3. MS [ESI]: m/z: [M+H]. calc. 734.2 obs. 734.7; ¹H NMR (400 MHz, CDCl₃): δ 0.88 (9H, m), 1.25 (45H, m), 1.56-1.69 (7H, m), 2.05 (4H, q, J=6.57 Hz), 2.27 (3H, m), 2.53 (1H, t, J=5.76 Hz), 2.77 (2H, t, J=5.91 Hz), 3.95 (2H, m), 4.06 (2H, t, J=6.68 Hz), 4.18 (2H, m), 4.67 (1H, m), 5.36 (4H, m), 6.37 (1H, d, J=7.17 Hz).

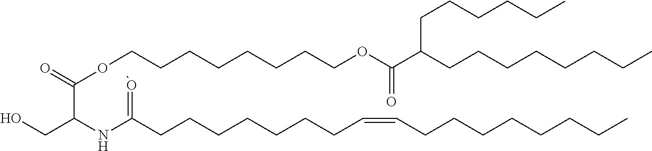

8-((Oleoylseryl)oxy)octyl 2-hexyldecanoate was synthesized according to representative synthetic scheme 1 and the corresponding methods. ¹H NMR (400 MHz, CDCl₃): δ 0.87 (9H, m), 1.25 (48H, m), 1.61 (10H, m), 2.01 (4H, m), 2.26 (3H, m), 3.95 (2H, d, J=3.76 Hz), 4.06 (2H, t, J=6.66 Hz), 4.18 (2H, m), 4.66 (1H, m), 5.33 (2H, m), 6.38 (1H, d, J=7.03 Hz). MS [ESI]: m/z: [M+H] calc. 736.6 obs. 736.7.

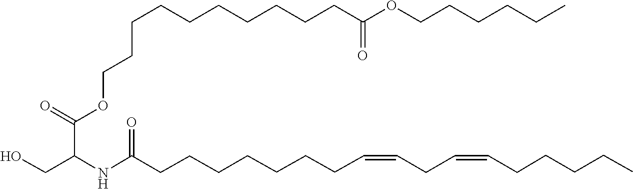 [SMILES: CCCCC/C=C\C/C=C\CCCCCCCC(=O)NC(CO)C(=O)OCCCCCCCCCCC(=O)OCCCCCC]

Hexyl 11-((((9Z,12Z)-octadeca-9,12-dienoyl)seryl)oxy)undecanoate was synthesized according to representative synthetic scheme 1 and the corresponding methods. ¹H NMR (400 MHz, CDCl₃): δ 0.89 (6H, t, J=7.02 Hz), 1.31 (32H, m), 1.63 (8H, m), 2.05 (4H, q, J=7.09 Hz), 2.27 (4H, m), 2.77 (2H, t, J=6.35 Hz), 3.95 (2H, d, J=3.76 Hz), 4.05 (2H, t, J=6.75 Hz), 4.18 (2H, m), 4.66 (1H, m), 5.35 (4H, m), 6.39 (1H, d, J=7.00 Hz). MS [ESI]: m/z: [M+H] calc. 636.5 obs. 636.6.

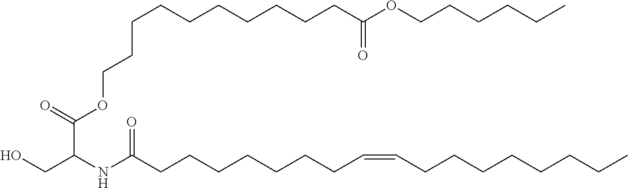 [SMILES: CCCCCCCC/C=C\CCCCCCCC(=O)NC(CO)C(=O)OCCCCCCCCCCC(=O)OCCCCCC]

Hexyl 11-((oleoylseryl)oxy)undecanoate was synthesized according to representative synthetic scheme 1 and the corresponding methods. ¹H NMR (400 MHz, CDCl₃): δ 0.89 (6H, m), 1.31 (38H, m), 1.63 (8H, m), 2.01 (4H, m), 2.29 (4H, m), 3.95 (2H, d, J=3.78 Hz), 4.06 (2H, t, J=6.74 Hz), 4.18 (2H, td, J=6.70, 3.14 Hz), 4.67 (1H, m), 5.34 (2H, m), 6.38 (1H, d, J=7.00 Hz). MS [ESI]: m/z: [M+H] calc. 638.5 obs. 638.6.

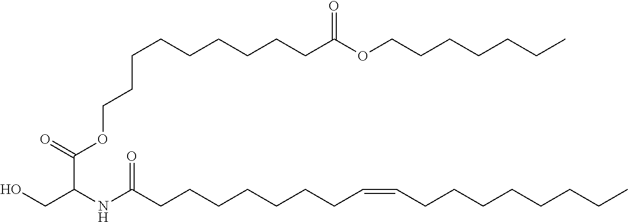 [SMILES: CCCCCCCC/C=C\CCCCCCCC(=O)NC(CO)C(=O)OCCCCCCCCCC(=O)OCCCCCCC]

Heptyl 10-((oleoylseryl)oxy)decanoate was synthesized according to representative synthetic scheme 1 and the corresponding methods. 1H NMR (400 MHz, CDCl₃): δ 0.88 (6H, m), 1.31 (38H, m), 1.60 (811, m), 2.01 (4H, q, J=6.36 Hz), 2.29 (4H, m), 3.95 (2H, m), 4.05 (2H, t, J=6.75 Hz), 4.18 (2H, m), 4.66 (1H, m), 5.34 (2H, m), 6.39 (1H, d, J=7.24 Hz). MS [ESI]: m/z: [M+H] calc. 638.5 obs. 638.6.

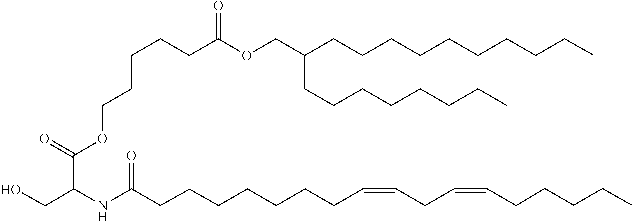 [SMILES: CCCCC/C=C\C/C=C\CCCCCCCC(=O)NC(CO)C(=O)OCCCCCC(=O)OCC(CCCCCCCC)CCCCCCCCCC]

2-Octyldodecyl 6-((((9Z,12Z)-octadeca-9,12-dienoyl)seryl)oxy)hexanoate was synthesized according to representative synthetic scheme 1 and the corresponding methods. 1H NMR (400 MHz, CDCl₃): δ 0.88 (911, m), 1.26 (48H, m), 1.65 (7H, m), 2.05 (4H, q, J=6.89 Hz), 2.32 (4H, m), 2.77 (2H, t, J=6.35 Hz), 3.97 (4H, m), 4.20 (2H, m), 4.67 (1H, m), 5.35 (4H, m), 6.40 (1H, d, J=7.26 Hz). MS [ESI]: m/z: [M+H] calc. 762.7 obs. 762.8.

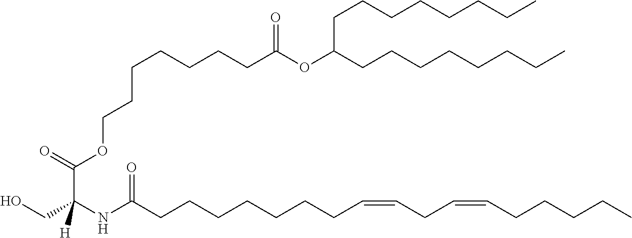 [SMILES: CCCCC/C=C\C/C=C\CCCCCCCC(=O)N[C@H](CO)C(=O)OCCCCCCCC(=O)OC(CCCCCCCC)CCCCCCCC]

Heptadecan-9-yl 8-((((9Z,12Z)-octadeca-9,12-dienoyl)-D-seryl)oxy)octanoate was synthesized according to representative synthetic scheme 1 and the corresponding methods. 1H NMR (400 MHz, CDCl₃): δ 0.87 (9H, m), 1.25 (44H, m), 1.50 (4H, m), 1.64 (6H, m), 2.04 (4H, q, J=6.72 Hz), 2.27 (4H, m), 2.77 (2H, t, J=6.30 Hz), 3.94 (2H, m), 4.18 (2H, m), 4.66 (1H, m), 4.86 (1H, quint, J=6.28 Hz), 5.35 (4H, m), 6.40 (1H, d, J=7.09 Hz). MS [ESI]: m/z: [M+H] calc. 748.6 obs. 748.6.

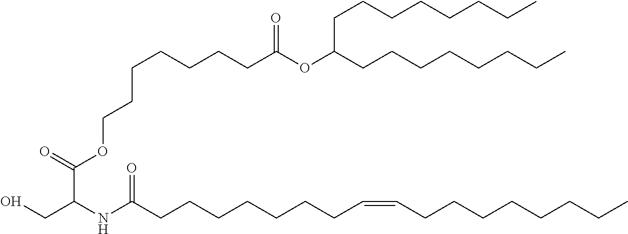 [SMILES: CCCCCCCC/C=C\CCCCCCCC(=O)NC(CO)C(=O)OCCCCCCCC(=O)OC(CCCCCCCC)CCCCCCCC]

Heptadecan-9-yl 8-((oleoylseryl)oxy)octanoate was synthesized according to representative synthetic scheme 1 and the corresponding methods. ¹H NMR (500 MHz, CDCl₃): δ 0.89 (9H, t, J=7.06 Hz), 1.27 (50H, m), 1.52 (4H, m), 1.65 (6H, m), 2.02 (4H, m), 2.29 (4H, m), 3.97 (2H, m), 4.19 (2H, m), 4.68 (1H, m), 4.88 (1H, i, J=6.26 Hz), 5.36 (2H, m), 6.42 (1H, d, J=7.05 Hz). MS [ESI]: m/z: [M+H] calc. 750.7 obs. 750.8.

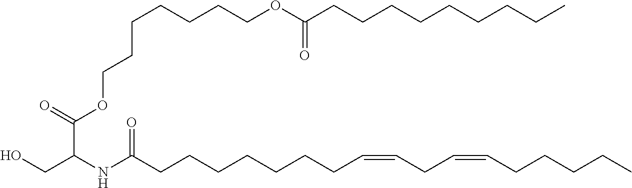 [SMILES: CCCCC/C=C\C/C=C\CCCCCCCC(=O)NC(CO)C(=O)OCCCCCCCOC(=O)CCCCCCCCC]

7-((((9Z,12Z)-Octadeca-9,12-dienoyl)seryl)oxy)heptyl decanoate was synthesized according to representative synthetic scheme 1 and the corresponding methods. ¹H NMR (400 MHz, CDCl₃): δ 0.88 (6H, m), 1.32 (32H, m), 1.58 (8H, m), 2.05 (4H, q, J=6.80 Hz), 2.29 (4H, m), 2.77 (2H, t, J=6.42 Hz), 3.95 (2H, m), 4.06 (2H, t, J=6.75 Hz), 4.18 (2H, m), 4.67 (1H, m), 5.35 (4H, m), 6.38 (1H, d, J=7.17 Hz). MS [ESI]: m/z: [M+H] calc. 636.5 obs. 636.7.

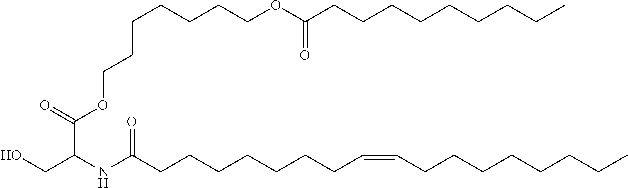 [SMILES: CCCCCCCC/C=C\CCCCCCCC(=O)NC(CO)C(=O)OCCCCCCCOC(=O)CCCCCCCCC]

7-((Oleoylseryl)oxy)heptyl decanoate was synthesized according to representative synthetic scheme 1 and the corresponding methods. 1H NMR (400 MHz, CDCl₃): δ 0.89 (6H, m), 1.29 (39H, m), 1.65 (711, m), 2.02 (4H, m), 2.29 (4H, dt, J=9.54, 7.73 Hz), 2.66 (1H, t, J=5.78 Hz), 3.96 (2H, m), 4.07 (2H, t, J=6.74 Hz), 4.20 (2H, m), 4.68 (1H, m), 5.36 (2H, m), 6.41 (1H, d, J=6.99 Hz). MS [ESI]: m/z: [M+H] calc. 638.5 obs. 638.6.

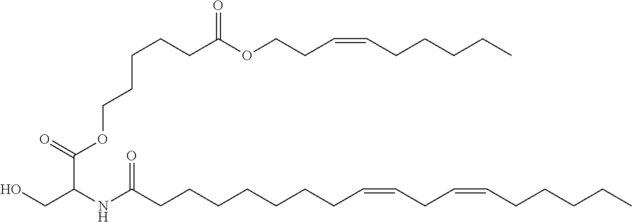 [SMILES: CCCCC/C=C\C/C=C\CCCCCCCC(=O)NC(CO)C(=O)OCCCCCC(=O)OCC/C=C\CCCCC]

(Z)-Non-3-en-1-yl 6-((((9Z,12Z)-octadeca-9,12-dienoyl)seryl)oxy)hexanoate was synthesized according to representative synthetic scheme 1 and the corresponding methods. ¹H NMR (400 MHz, CDCl₃): δ 0.89 (6H, t, J=6.85 Hz), 1.32 (22H, m), 1.60-1.73 (6H, m), 2.04 (6H, quint, J=6.43 Hz), 2.30 (6H, m), 2.69 (1H, t, J=5.70 Hz), 2.77 (2H, t, J=5.98 Hz), 3.95 (2H, m), 4.07 (2H, t, J=6.93 Hz), 4.13-4.27 (2H, 4.20 (q, J=10.85 Hz), 4.20 (td, J=12.88, 1.94 Hz)), 4.67 (1H, dt, J=7.17, 3.57 Hz), 5.35 (5H, m), 5.49 (1H, m), 6.39 (1H, d, J=7.65 Hz). MS [ESI]: m/z: [M+H] calc. 606.5 obs. 606.4.

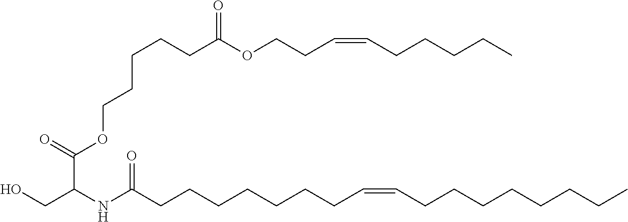 [SMILES: CCCCC/C=C\CCOC(=O)CCCCCOC(=O)C(CO)NC(=O)CCCCCCC/C=C\CCCCCCCC]

(Z)-Non-3-en-1-yl 6-((oleoylseryl)oxy)hexanoate was synthesized according to representative synthetic scheme 1 and the corresponding methods. ¹H NMR (500 MHz, CDCl₃): δ 0.84 (6H, m), 1.25 (31H, m), 1.61 (6H, m), 1.97 (6H, m), 2.21 (2H, dd, J=7.88, 7.48 Hz), 2.27 (2H, t, J=7.23 Hz), 2.33 (2H, q, J=7.04 Hz), 3.86 (1H, m), 3.92 (1H, m), 4.02 (2H, t, J=6.92 Hz), 4.14 (2H, m), 4.62 (1H, dt, J=7.27, 3.63 Hz), 5.26-5.31 (3H, m), 5.46 (1H, m), 6.53 (1H, m). MS [ESI]: m/z: [M+H] calc. 608.5 obs. 608.6.

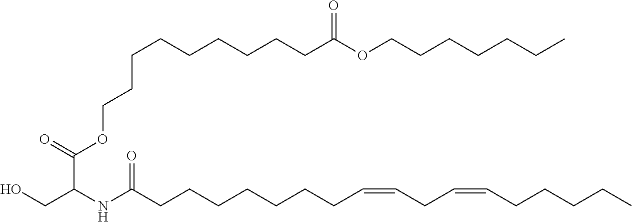 [SMILES: CCCCC/C=C\C/C=C\CCCCCCCC(=O)NC(CO)C(=O)OCCCCCCCCCC(=O)OCCCCCCC]

Heptyl 10-((((9Z,12Z)-octadeca-9,12-dienoyl)seryl)oxy)decanoate was synthesized according to representative synthetic scheme 1 and the corresponding methods. ¹H NMR (400 MHz, CDCl₃): δ 0.88 (6H, td, J=6.88, 1.89 Hz), 1.31 (33H, m), 1.63 (8H, m), 2.04 (4H, q, J=7.00 Hz), 2.27 (4H, q, J=7.65 Hz), 2.77 (2H, m), 3.95 (2H, d, J=3.79 Hz), 4.05 (2H, t, J=6.74 Hz), 4.18 (2H, m), 4.66 (1H, m), 5.35 (4H, m), 6.40 (1H, d, J=6.83 Hz). MS [ESI]: m/z: [M+H] calc. 636.5 obs. 636.4.

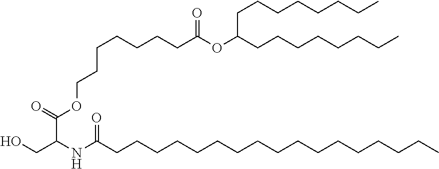 [SMILES: CCCCCCCCCCCCCCCCCC(=O)NC(CO)C(=O)OCCCCCCCC(=O)OC(CCCCCCCC)CCCCCCCC]

Heptadecan-9-yl 8-((stearoylseryl)oxy)octanoate was synthesized according to representative synthetic scheme 1 and the corresponding methods. ¹H NMR (400 MHz, CDCl₃): δ 0.88 (9H, m), 1.25 (57H, m), 1.47-1.53 (4H, m), 1.59-1.69 (8H, m), 2.27 (4H, dt, J=7.57, 5.31 Hz), 3.95 (2H, m), 4.14 (2H, m), 4.67 (1H, m), 4.86 (1H, i, J=6.21 Hz), 6.38 (1H, m). MS [ESI]: m/z: [M+H] calc. 752.7 obs. 752.8.

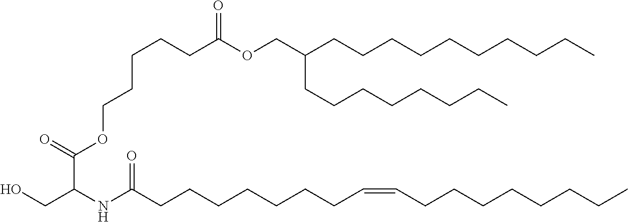 [SMILES: CCCCCCCC/C=C\CCCCCCCC(=O)NC(CO)C(=O)OCCCCCC(=O)OCC(CCCCCCCC)CCCCCCCCCC]

2-Octyldodecyl 6-((oleoylseryl)oxy)hexanoate was synthesized according to representative synthetic scheme 1 and the corresponding methods. ¹H NMR (500 MHz, CDCl₃): δ 0.86 (9H, t, J=6.92 Hz), 1.24 (53H, m), 1.60-1.70 (6H, m), 1.96-2.01 (4H, m), 2.24 (2H, dd, J=8.33, 6.95 Hz), 1.34-1.41 (2H, m), 2.30 (2H, t, J=7.27 Hz), 2.74 (1H, m, J=0.68 Hz), 3.95 (4H, m), 4.13 (1H, m), 4.22 (1H, m), 4.63-4.67 (1H, m), 5.32 (2H, m), 6.39 (1H, m). MS [ESI]: m/z: [M+H] calc. 764.7 obs. 764.9.

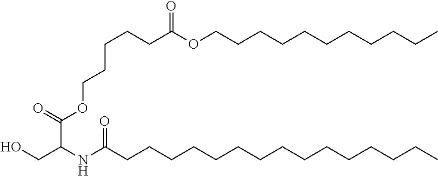 [SMILES: CCCCCCCCCCCCCCCC(=O)NC(CO)C(=O)OCCCCCC(=O)OCCCCCCCCCCC]

Undecyl 6-((palmitoylseryl)oxy)hexanoate was synthesized according to representative synthetic scheme 1 and the corresponding methods. ¹H NMR (400 MHz, CDCl₃): δ 0.90 (6H, t, J=6.81 Hz), 1.27 (45H, m), 1.59-1.72 (7H, m), 2.33 (4H, m), 3.99 (2H, m), 4.07 (2H, t, J=6.75 Hz) 4.19 (1H, m), 4.25 (1H, m), 4.69 (1H, i, J=3.52 Hz), 6.43 (1H, m). MS [ESI]: m/z: [M+H] calc. 612.5 obs. 612.3.

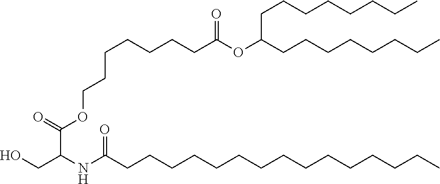 [SMILES: CCCCCCCCCCCCCCCC(=O)NC(CO)C(=O)OCCCCCCCC(=O)OC(CCCCCCCC)CCCCCCCC]

Heptadecan-9-yl 8-((palmitoylseryl)oxy)octanoate was synthesized according to representative synthetic scheme 1 and the corresponding methods. ¹H NMR (400 MHz, CDCl₃): δ 0.88 (9H, t, J=6.62 Hz), 1.25 (55H, s), 1.46-1.54 (4H, m), 1.59-1.69 (6H, m), 2.24-2.30 (4H, m), 3.95 (2H, m), 4.18 (2H, m), 4.67 (1H, m), 4.86 (1H, quint, J=6.23 Hz), 6.40 (1H, m). MS [ESI]: m/z: [M+H] calc. 724.6 obs. 724.7.

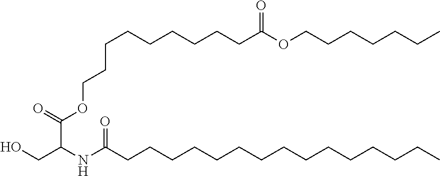 [SMILES: CCCCCCCCCCCCCCCC(=O)NC(CO)C(=O)OCCCCCCCCCC(=O)OCCCCCCC]

Heptyl 10-((palmitoylseryl)oxy)decanoate was synthesized according to representative synthetic scheme 1 and the corresponding methods. ¹H NMR (400 MHz, CDCl₃): δ 0.89 (6H, m), 1.27 (44H, m), 1.64 (6H, m), 2.29 (4H, m), 3.96 (2H, m), 4.07 (2H, t, J=6.75 Hz), 4.68 (1H, m), 6.43 (1H, d, J=6.96 Hz), 4.13 (1H, q, J=6.83 Hz), 4.17-4.22 (2H, m). MS [ESI]: m/z: [M+H] calc. 612.5 obs. 612.3.

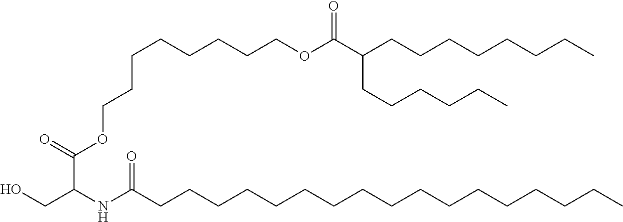 [SMILES: CCCCCCCCCCCCCCCCCC(=O)NC(CO)C(=O)OCCCCCCCCOC(=O)C(CCCCCC)CCCCCCCC]

8-((stearoylseryl)oxy)octyl 2-hexyldecanoate was synthesized according to representative synthetic scheme 1 and the corresponding methods. ¹H NMR (500 MHz, CDCl₃): δ 0.88 (9H, m), 1.25 (56H, m), 1.61 (10H, m), 2.27 (3H, m), 3.95 (2H, d, J=3.74 Hz), 4.06 (2H, t, J=6.79 Hz), 4.18 (2H, m), 4.66 (1H, m), 6.38 (1H, d, J=6.95 Hz). MS [ESI]: m/z: [M+H] calc. 738.7 obs. 738.8.

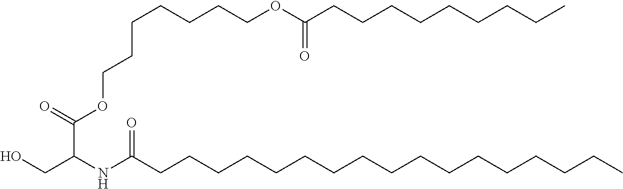 [SMILES: CCCCCCCCCCCCCCCCCC(=O)NC(CO)C(=O)OCCCCCCCOC(=O)CCCCCCCCC]

7-((Stearoylseryl)oxy)heptyl decanoate was synthesized according to representative synthetic scheme 1 and the corresponding methods. ¹H NMR (400 MHz, CDCl₃): δ 0.88 (6H, t, J=7.16 Hz), 1.26 (46H, m), 1.65 (8H, m), 2.28 (4H, q, J=8.33 Hz), 2.58 (1H, t, J=5.58 Hz), 3.95 (2H, t, J=4.40 Hz), 4.06 (2H, t, J=6.82 Hz), 4.19 (2H, m), 4.67 (1H, m), 6.38 (1H, d, J=7.17 Hz). MS [ESI]: m/z: [M+H] calc. 640.6 obs. 640.6.

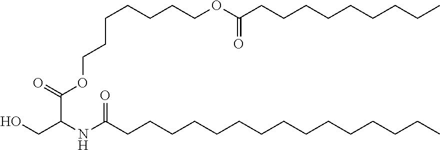 [SMILES: CCCCCCCCCCCCCCCC(=O)NC(CO)C(=O)OCCCCCCCOC(=O)CCCCCCCCC]

7-((Palmitoylseryl)oxy)heptyl decanoate was synthesized according to representative synthetic scheme 1 and the corresponding methods. ¹H NMR (400 MHz, CDCl₃): δ 0.88 (6H, t, J=0.86 Hz), 1.25 (42H, m), 1.65 (8H, m), 2.27 (4H, m), 2.59 (1H, t, J=5.72 Hz), 3.95 (2H, m), 4.06 (2H, t, J=6.75 Hz), 4.19 (2H, m), 4.67 (1H, m), 6.38 (1H, d, J=6.98 Hz). MS [ESI]: m/z: [M+H] calc. 612.5 obs. 612.3.

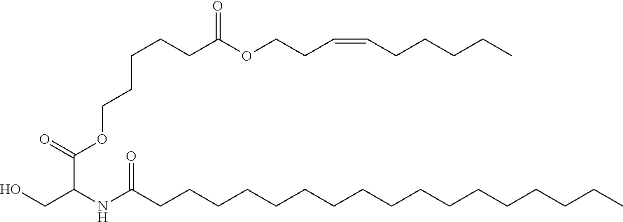 [SMILES: CCCCC/C=C\CCOC(=O)CCCCCOC(=O)C(CO)NC(=O)CCCCCCCCCCCCCCCCC]

(Z)-Non-3-en-1-yl 6-((stearoylseryl)oxy)hexanoate was synthesized according to representative synthetic scheme 1 and the corresponding methods. ¹H NMR (400 MHz, CDCl₃): δ 0.88 (6H, m), 1.25 (36H, m), 1.67 (6H, m), 2.03 (2H, q, J=7.25 Hz), 2.31 (6H, m), 2.71 (1H, t, J=5.80 Hz), 3.95 (2H, m), 4.07 (2H, t, J=6.98 Hz), 4.17 (1H, m), 4.23 (1H, m), 4.67 (1H, m), 5.33 (1H, m), 5.50 (1H, m), 6.40 (1H, d, J=7.17 Hz). MS [ESI]: m/z: [M+H] calc. 610.5 obs. 610.6.

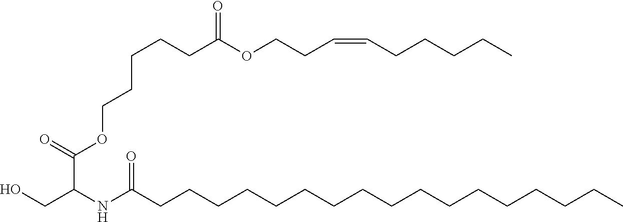 [SMILES: CCCCC/C=C\CCOC(=O)CCCCCOC(=O)C(CO)NC(=O)CCCCCCCCCCCCCCCCC]

(Z)-non-3-en-1-yl 6-((stearoylseryl)oxy)hexanoate was synthesized according to representative synthetic scheme 1 and the corresponding methods. ¹H NMR (400 MHz, CDCl₃): ¹H NMR (400 MHz): δ 0.88 (6H, m), 1.25 (36H, m), 1.67 (6H, m), 2.03 (2H, q, J=7.25 Hz), 2.31 (6H, m), 2.71 (1H, t, J=5.80 Hz), 3.95 (2H, m), 4.07 (2H, t, J=6.98 Hz), 4.17 (1H, m), 4.23 (1H, m), 4.67 (1H, m), 5.33 (1H, m), 5.50 (1H, m), 6.40 (1H, d, J=7.17 Hz). MS [ESI]: m/z: [M+H] calc. 610.5 obs. 610.6.

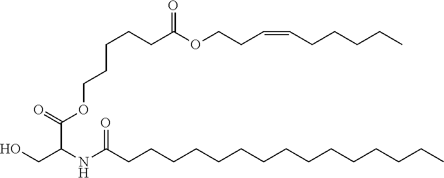 [SMILES: CCCCC/C=C\CCOC(=O)CCCCCOC(=O)C(CO)NC(=O)CCCCCCCCCCCCCCC]

(Z)-non-3-en-1-yl 6-((palmitoylseryl)oxy)hexanoate was synthesized according to representative synthetic scheme 1 and the corresponding methods. ¹H NMR (400 MHz, CDCl₃): δ 0.89 (6H, m), 1.26 (32H, m), 1.60-1.75 (6H, m), 2.03 (2H, qd, J=7.26, 1.37 Hz), 2.32 (6H, m), 2.69 (1H, t, J=5.72 Hz), 3.89-4.02 (2H, m), 4.07 (2H, t, J=6.92 Hz), 4.18 (2H, m), 4.67 (1H, i, J=3.62 Hz), 5.33 (1H, m), 5.51 (1H, m), 6.39 (1H, d, J=7.26 Hz). MS [ESI]: m/z: [M+H] calc. 582.5 obs. 582.4.

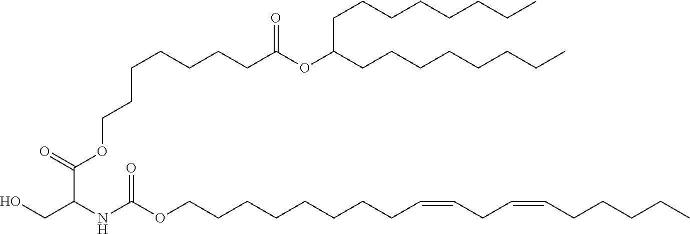 [SMILES: CCCCC/C=C\C/C=C\CCCCCCCCOC(=O)NC(CO)C(=O)OCCCCCCCC(=O)OC(CCCCCCCC)CCCCCCCC]

heptadecan-9-yl 8-((((((9Z,12Z)-octadeca-9,12-dien-1-yl)oxy)carbonyl)seryl)oxy)octanoate was synthesized according to representative synthetic scheme 2 and the corresponding methods. ¹H NMR (400 MHz, CDCl₃): δ 0.88 (9H, m), 1.26 (46H, m), 1.44-1.55 (4H, m), 1.58 (6H, m), 2.04 (4H, m), 2.27 (2H, m), 2.77 (2H, dd, J=7.51, 6.51 Hz), 3.95 (2H, m), 4.07 (4H, m), 4.41 (1H, m), 4.86 (1H, i, J=6.23 Hz), 5.36 (4H, m), 5.57 (1H, s). MS [ESI]: m/z: [M+H] calc. 778.6 obs. 778.8.

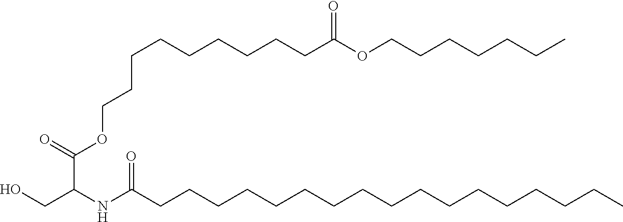 [SMILES: CCCCCCCCCCCCCCCCCC(=O)NC(CO)C(=O)OCCCCCCCCCC(=O)OCCCCCCC]

heptyl 10-((stearoylseryl)oxy)decanoate was synthesized according to representative synthetic scheme 1 and the corresponding methods. ¹H NMR (400 MHz, CDCl₃): δ 0.88 (6H, m), 1.25 (44H, m), 1.57 (10H, m), 2.29 (4H, m), 3.95 (2H, dd, J=5.21, 3.67 Hz), 4.06 (2H, t, J=6.75 Hz), 4.18 (2H, m), 4.67 (1H, m), 6.38 (1H, d, J=6.39 Hz). MS [ESI]: m/z: [M+H] calc. 640.5 obs. 640.4.

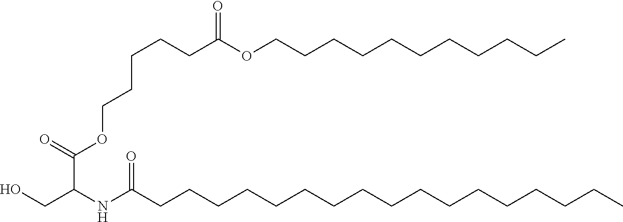 [SMILES: CCCCCCCCCCCCCCCCCC(=O)NC(CO)C(=O)OCCCCCC(=O)OCCCCCCCCCCC]

undecyl 6-((stearoylseryl)oxy)hexanoate was synthesized according to representative synthetic scheme 1 and the corresponding methods. ¹H NMR (400 MHz, CDCl₃): δ 0.88 (6H, t, J=6.79 Hz), 1.25 (40H, m), 1.37-1.45 (2H, m), 1.55-1.74 (12H, m), 2.26 (2H, dd, J=8.23, 7.05 Hz), 2.31 (2H, t, J=7.29 Hz), 3.93 (2H, m), 4.05 (2H, t, J=6.73 Hz), 4.17 (2H, t, J=6.63 Hz), 4.67 (1H, m), 6.41 (1H, d, J=7.05 Hz). MS [ESI]: m/z: [M+H] calc. 640.5 obs. 640.7.

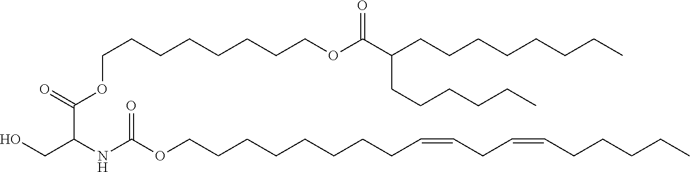 [SMILES: CCCCC/C=C\C/C=C\CCCCCCCCOC(=O)NC(CO)C(=O)OCCCCCCCCOC(=O)C(CCCCCC)CCCCCCCC]

8-((((((9Z,12Z)-octadeca-9,12-dien-1-yl)oxy)carbonyl)seryl)oxy)octyl 2-hexyldecanoate was synthesized according to representative synthetic scheme 2 and the corresponding methods. ¹H NMR (400 MHz, CDCl₃): δ 0.88 (9H, m), 1.25 (44H, m), 1.48-1.70 (10H, m), 2.05 (5H, m), 2.77 (2H, dd, J=7.74, 6.60 Hz), 3.64 (1H, t, J=6.62 Hz), 3.95 (2H, m), 4.07 (3H, m), 4.18 (2H, td, J=6.70, 2.65 Hz), 4.41 (1H, m), 5.36 (5H, m), 5.51-5.61 (1H, m). MS [ESI]: m/z: [M+H] calc. 764.6 obs. 764.7.

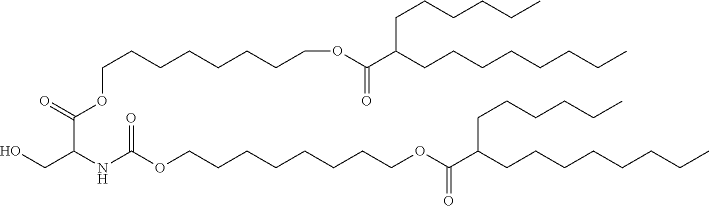 [SMILES: CCCCCCCCC(CCCCCC)C(=O)OCCCCCCCCOC(=O)NC(CO)C(=O)OCCCCCCCCOC(=O)C(CCCCCC)CCCCCCCC]

25-hexyl-11-(hydroxymethyl)-10,13,24-trioxo-9,14,23-trioxa-12-azatritriacontyl 2-hexyldecanoate was synthesized according to representative synthetic scheme 2 and the corresponding methods. ¹H NMR (400 MHz, CDCl₃): δ 0.87 (12H, t, J=6.58 Hz), 1.25 (56H, m), 1.58 (16H, m), 2.30 (2H, m), 3.95 (2H, m), 4.06 (6H, m), 4.17 (2H, m), 4.36-4.46 (1H, m), 5.57 (1H, m). MS [ESI]: m/z: [M+H] calc. 882.7 obs. 882.8.

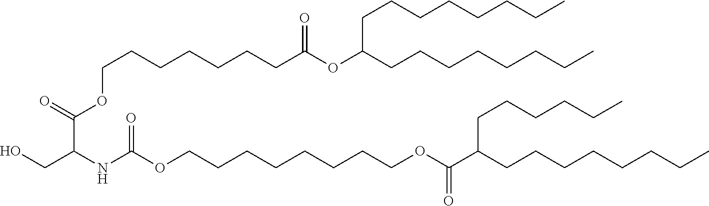 [SMILES: CCCCCCCCC(CCCCCCCC)OC(=O)CCCCCCCOC(=O)C(CO)NC(=O)OCCCCCCCCOC(=O)C(CCCCCC)CCCCCCCC]

12-(hydroxymethyl)-24-octyl-10,13,22-trioxo-9,14,23-trioxa-11-azadotriacontyl 2-hexyldecanoate was synthesized according to representative synthetic scheme 2 and the corresponding methods. ¹H NMR (400 MHz, CDCl₃): δ 0.87 (12H, t, J=6.58 Hz), 1.25 (56H, m), 1.45-1.71 (18H, m), 2.27 (3H, m), 3.97 (2H, m), 4.07 (4H, m), 4.18 (2H, m), 4.40 (1H, s), 4.86 (1H, i, J=6.34 Hz), 5.58 (1H, m). MS [ESI]: m/z: [M+H] calc. 896.7 obs. 896.8.

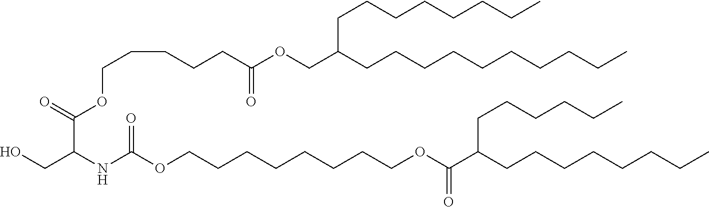 [SMILES: CCCCCCCCCCC(CCCCCCCC)COC(=O)CCCCCOC(=O)C(CO)NC(=O)OCCCCCCCCOC(=O)C(CCCCCC)CCCCCCCC]

12-(hydroxymethyl)-23-octyl-10,13,20-trioxo-9,14,21-trioxa-11-azatritriacontyl 2-hexyldecanoate was synthesized according to representative synthetic scheme 2 and the corresponding methods. ¹H NMR (400 MHz, CDCl₃): δ 0.87 (12H, m), 1.26 (62H, m), 1.62 (13H, m), 2.31 (3H, m), 4.06 (11H, m), 4.41 (1H, br s), 5.59 (1H, d, J=7.52 Hz). MS [ESI]: m/z: [M+H] calc. 910.8 obs. 910.8.

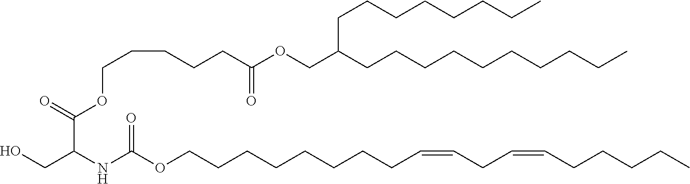 [SMILES: CCCCC/C=C\C/C=C\CCCCCCCCOC(=O)NC(CO)C(=O)OCCCCCC(=O)OCC(CCCCCCCC)CCCCCCCCCC]

h 2-octyldodecyl 6-((((((9Z,12Z)-octadeca-9,12-dien-1-yl)oxy)carbonyl)seryl)oxy)hexanoate was synthesized according to representative synthetic scheme 2 and the corresponding methods. ¹H NMR (400 MHz, CDCl₃): δ 0.90 (9H, m), 1.28 (47H, m), 1.65 (10H, m), 2.06 (4H, q, J=7.00 Hz), 2.33 (2H, t, J=7.26 Hz), 2.79 (2H, dd, J=7.67, 6.57 Hz), 3.99 (4H, m), 4.09 (2H, t, J=6.98 Hz), 4.21 (2H, m), 4.42 (1H, d, J=7.86 Hz), 5.37 (4H, m), 5.60 (1H, d, J=7.52 Hz). MS [ESI]: m/z: [M+H] calc. 792.7 obs. 792.7.

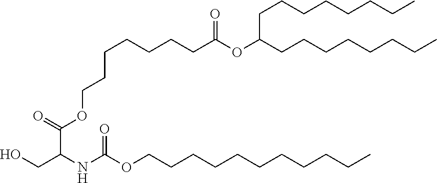 [SMILES: CCCCCCCCCCCOC(=O)NC(CO)C(=O)OCCCCCCCC(=O)OC(CCCCCCCC)CCCCCCCC]

heptadecan-9-yl 8-((((undecyloxy)carbonyl)seryl)oxy)octanoate was synthesized according to representative synthetic scheme 2 and the corresponding methods. ¹H NMR (400 MHz, CDCl₃): δ 0.88 (9H, m), 1.26 (46H, m), 1.62 (10H, m), 2.27 (2H, t, J=7.86 Hz), 3.64 (1H, t, J=6.66 Hz), 3.97 (2H, m), 4.07 (4H, m), 4.41 (1H, br s), 4.86 (111, i, J=6.49 Hz), 5.57 (1H, m). MS [ESI]: m/z: [M+H] calc. 684.6 obs. 684.4.

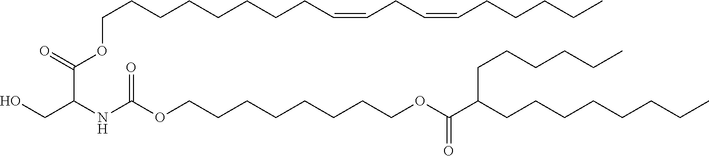 [SMILES: CCCCC/C=C\C/C=C\CCCCCCCCOC(=O)C(CO)NC(=O)OCCCCCCCCOC(=O)C(CCCCCC)CCCCCCCC]

8-(((3-hydroxy-1-(((9Z,12Z)-octadeca-9,12-dien-1-yl)oxy)-1-oxopropan-2-yl)carbamoyl)oxy)octyl 2-hexyldecanoate was synthesized according to representative synthetic scheme 2 and the corresponding methods. 1H NMR (400 MHz, CDCl₃): δ 0.88 (9H, m), 1.25 (46H, m), 1.59 (8H, m), 2.05 (3H, q, J=7.34 Hz), 2.17-2.26 (1H, m), 2.26-2.35 (1H, m), 2.77 (2H, dd, J=7.40, 6.56 Hz), 3.95 (2H, m), 4.07 (4H, m), 4.18 (2H, t, J=6.93 Hz), 4.41 (1H, m), 5.35 (4H, m), 5.51-5.62 (1H, m). MS [ESI]: m/z: [M+H] calc. 764.6 obs. 764.8.

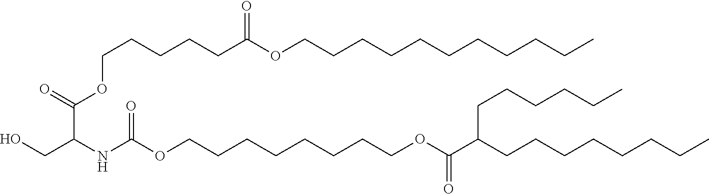 [SMILES: CCCCCCCCCCCOC(=O)CCCCCOC(=O)C(CO)NC(=O)OCCCCCCCCOC(=O)C(CCCCCC)CCCCCCCC]

12-(hydroxymethyl)-10,13,20-trioxo-9,14,21-trioxa-11-azadotriacontyl 2-hexyldecanoate was synthesized according to representative synthetic scheme 2 and the corresponding methods. ¹H NMR (400 MHz, CDCl₃): δ 0.87 (9H, t, J=6.49 Hz), 1.26 (44H, m), 1.58 (16H, m), 2.31 (3H, t, J=7.69 Hz), 2.37-2.50 (1H, m), 4.05 (8H, m), 4.20 (2H, m), 4.40 (1H, m), 5.59 (1H, m). MS [ESI]: m/z: [M+H] calc. 784.6 obs. 784.7.

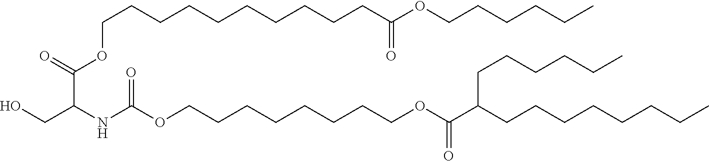 [SMILES: CCCCCCCCC(CCCCCC)C(=O)OCCCCCCCCOC(=O)NC(CO)C(=O)OCCCCCCCCCCC(=O)OCCCCCC]

hexyl 11-(((((8-((2-hexyldecanoyl)oxy)octyl)oxy)carbonyl)seryl)oxy)undecanoate was synthesized according to representative synthetic scheme 2 and the corresponding methods. ¹H NMR (400 MHz, CDCl₃): δ 0.88 (9H, m), 1.25 (48H, m), 1.57 (12H, m), 2.29 (3H, m), 3.96 (2H, m), 4.06 (6H, m), 4.18 (2H, td, J=6.66, 2.73 Hz), 4.41 (1H, m), 5.56 (1H, m). MS [ESI]: m/z: [M+H] calc. 784.6 obs. 784.6.

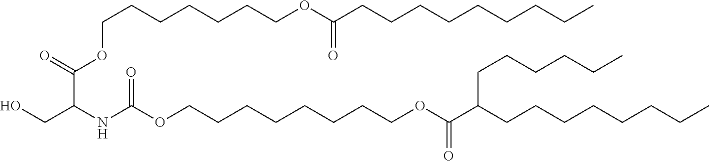 [SMILES: CCCCCCCCCC(=O)OCCCCCCCOC(=O)C(CO)NC(=O)OCCCCCCCCOC(=O)C(CCCCCC)CCCCCCCC]

12-(hydroxymethyl)-10,13,23-trioxo-9,14,22-trioxa-11-azadotriacontyl 2-hexyldecanoate was synthesized according to representative synthetic scheme 2 and the corresponding methods. ¹H NMR (400 MHz, CDCl₃): δ 0.87 (9H, m), 1.25 (46H, m), 1.59 (14H, m), 2.29 (3H, m), 3.95 (2H, m), 4.06 (6H, m), 4.18 (2H, m), 4.42 (1H, m), 5.58 (1H, m). MS [ESI]: m/z: [M+H] calc. 784.6 obs. 784.7.

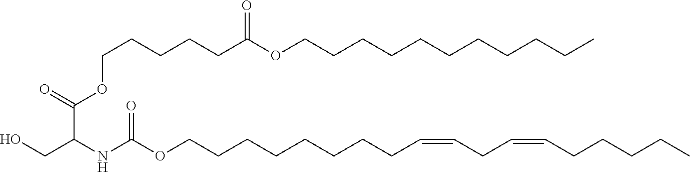 [SMILES: CCCCC/C=C\C/C=C\CCCCCCCCOC(=O)NC(CO)C(=O)OCCCCCC(=O)OCCCCCCCCCCC]

undecyl 6-((((((9Z,12Z)-octadeca-9,12-dien-1-yl)oxy)carbonyl)seryl)oxy)hexanoate was synthesized according to representative synthetic scheme 2 and the corresponding methods. ¹H NMR (400 MHz, CDCl₃): δ 0.88 (6H, m), 1.26 (34H, m), 1.61 (8H, m), 2.05 (4H, q, J=7.36 Hz), 2.31 (2H, t, J=7.45 Hz), 2.77 (2H, t, J=6.32 Hz), 3.95 (2H, m), 4.06 (4H, m), 4.20 (2H, m), 4.40 (1H, m), 5.36 (4H, m), 5.58 (1H, m). MS [ESI]: m/z: [M+H] calc. 666.5 obs. 666.5.

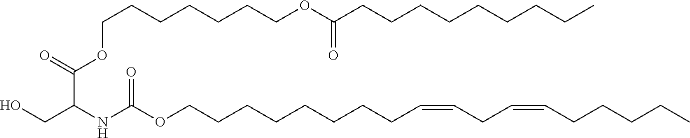 [SMILES: CCCCC/C=C\C/C=C\CCCCCCCCOC(=O)NC(CO)C(=O)OCCCCCCCOC(=O)CCCCCCCCC]

7-((((((9Z,12Z)-octadeca-9,12-dien-1-yl)oxy)carbonyl)seryl)oxy)heptyl decanoate was synthesized according to representative synthetic scheme 2 and the corresponding methods. ¹H NMR (400 MHz, CDCl₃): δ 0.88 (6H, m), 1.30 (34H, m), 1.58 (8H, m), 2.05 (4H, q, J=6.93 Hz), 2.29 (2H, t, J=7.68 Hz), 2.77 (2H, t, J=6.29 Hz), 3.95 (2H, m), 4.06 (4H, m), 4.18 (2H, td, J=6.49, 2.56 Hz), 4.41 (1H, m), 5.35 (4H, m), 5.57 (1H, m). MS [ESI]: m/z: [M+H] calc. 666.5 obs. 666.5.

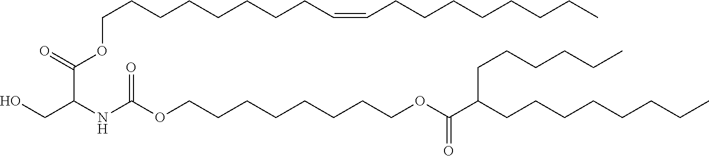 [SMILES: CCCCCCCC/C=C\CCCCCCCCOC(=O)C(CO)NC(=O)OCCCCCCCCOC(=O)C(CCCCCC)CCCCCCCC]

(Z)-8-(((3-hydroxy-1-(octadec-9-en-1-yloxy)-1-oxopropan-2-yl)carbamoyl)oxy)octyl 2-hexyldecanoate was synthesized according to representative synthetic scheme 2 and the corresponding methods. ¹H NMR (400 MHz, CDCl₃): δ 0.87 (9H, m), 1.25 (52H, m), 1.57 (8H, m), 2.02 (4H, m), 2.31 (1H, m), 3.95 (2H, m), 4.07 (4H, m), 4.18 (2H, t, J=6.63 Hz), 4.40 (1H, m), 5.35 (2H, m), 5.57 (1H, m). MS [ESI]: m/z: [M+H] calc. 766.5 obs. 766.7.

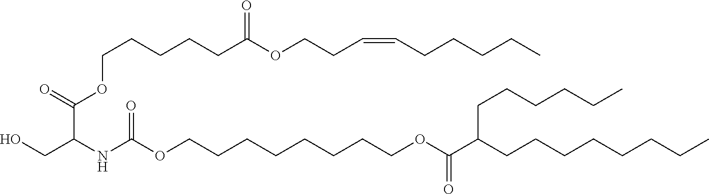 [SMILES: CCCCC/C=C\CCOC(=O)CCCCCOC(=O)C(CO)NC(=O)OCCCCCCCCOC(=O)C(CCCCCC)CCCCCCCC]

(Z)-12-(hydroxymethyl)-10,13,20-trioxo-9,14,21-trioxa-11-azatriacont-24-en-1-yl 2-hexyldecanoate was synthesized according to representative synthetic scheme 2 and the corresponding methods. ¹H NMR (400 MHz, CDCl₃): δ 0.88 (9H, m), 1.25 (36H, m), 1.62 (12H, m), 2.03 (2H, q, J=7.36 Hz), 2.31 (3H, m), 2.37 (2H, q, J=7.29 Hz), 3.91 (1H, m), 3.99 (1H, dd, J=11.15, 3.46 Hz), 4.06 (6H, m), 4.16 (1H, m), 4.24 (1H, m), 4.41 (1H, m), 5.33 (1H, m), 5.50 (1H, m), 5.60 (1H, m). MS [ESI]: m/z: [M+H] calc. 754.6 obs. 754.6.

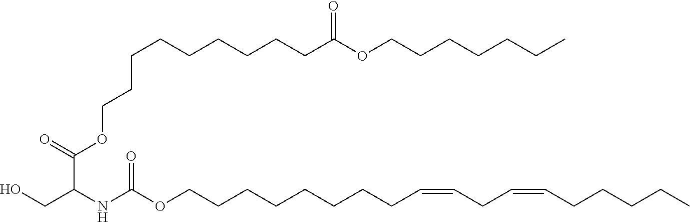 [SMILES: CCCCC/C=C\C/C=C\CCCCCCCCOC(=O)NC(CO)C(=O)OCCCCCCCCCC(=O)OCCCCCCC]

heptyl 10-((((((9Z,12Z)-octadeca-9,12-dien-1-yl)oxy)carbonyl)seryl)oxy)decanoate was synthesized according to representative synthetic scheme 2 and the corresponding methods. ¹H NMR (400 MHz, CDCl₃): δ 0.88 (6H, t, J=6.53 Hz), 1.30 (32H, m), 1.61 (10H, m), 2.05 (4H, q, J=7.34 Hz), 2.29 (2H, t, J=7.56 Hz), 2.77 (2H, t, J=6.60 Hz), 3.95 (2H, m), 4.06 (4H, q, J=6.80 Hz), 4.18 (2H, td, J=6.92, 2.05 Hz), 4.35-4.46 (1H, m), 5.35 (4H, m), 5.51-5.62 (1H, m). MS [ESI]: m/z: [M+H] calc. 666.5 obs. 666.6.

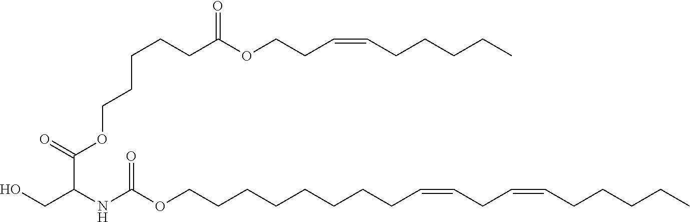 [SMILES: CCCCC/C=C\C/C=C\CCCCCCCCOC(=O)NC(CO)C(=O)OCCCCCC(=O)OCC/C=C\CCCCC]

(Z)-non-3-en-1-yl 6-((((((9Z,12Z)-octadeca-9,12-dien-1-yl)oxy)carbonyl)seryl)oxy)hexanoate was synthesized according to representative synthetic scheme 2 and the corresponding methods. ¹H NMR (400 MHz, CDCl₃): δ 0.89 (6H, t, J=6.58 Hz), 1.30 (24H, m), 1.64 (6H, m), 2.03 (6H, i, J=7.60 Hz), 2.31 (4H, m), 2.40-2.52 (1H, m), 2.77 (2H, t, J=6.41 Hz), 3.85-4.02 (2H, m), 4.06 (4H, td, J=7.20, 2.62 Hz), 4.18 (2H, m), 4.41 (1H, m), 5.34 (5H, m), 5.44-5.55 (1H, m), 5.55-5.67 (1H, m). MS [ESI]: m/z: [M+H] calc. 636.5 obs. 636.3.

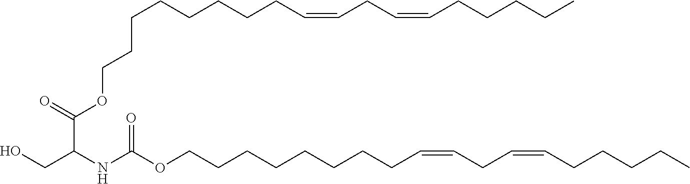 [SMILES: CCCCC/C=C\C/C=C\CCCCCCCCOC(=O)NC(CO)C(=O)OCCCCCCCC/C=C\C/C=C\CCCCC]

(9Z,12Z)-octadeca-9,12-dien-1-yl ((((9Z,12Z)-octadeca-9,12-dien-1-yl)oxy)carbonyl)serinate was synthesized according to representative synthetic scheme 2 and the corresponding methods. ¹H NMR (400 MHz, CDCl₃): δ 0.89 (6H, t, J=6.70 Hz), 1.30 (32H, m), 1.64 (4H, m), 2.04 (8H, q, J=6.69 Hz), 2.77 (4H, t, J=6.35 Hz), 3.95 (2H, m), 4.07 (2H, t, J=6.72 Hz), 4.18 (2H, t, J=6.70 Hz), 4.41 (1H, m), 5.36 (8H, m), 5.49-5.62 (1H, m). MS [ESI]: m/z: [M+H] calc. 646.5 obs. 646.5.

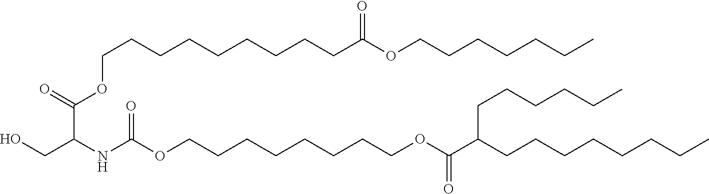 [SMILES: CCCCCCCCC(CCCCCC)C(=O)OCCCCCCCCOC(=O)NC(CO)C(=O)OCCCCCCCCCC(=O)OCCCCCCC]

heptyl 10-(((((8-((2-hexyldecanoyl)oxy)octyl)oxy)carbonyl)seryl)oxy)decanoate was synthesized according to representative synthetic scheme 2 and the corresponding methods. ¹H NMR (400 MHz, CDCl₃): δ 0.87 (9H, m), 1.25 (48H, m), 1.61 (12H, m), 2.29 (3H, m), 3.96 (2H, m), 4.05 (6H, m), 4.18 (2H, m), 4.41 (1H, m), 5.58 (1H, m). MS [ESI]: m/z: [M+H] calc. 784.6 obs. 784.7.

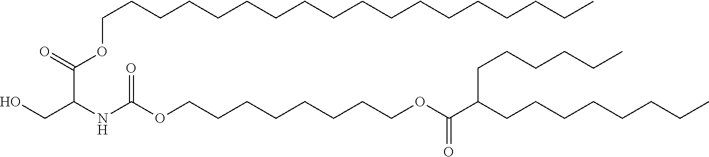 [SMILES: CCCCCCCCCCCCCCCCCCOC(=O)C(CO)NC(=O)OCCCCCCCCOC(=O)C(CCCCCC)CCCCCCCC]

8-(((3-hydroxy-1-(octadecyloxy)-1-oxopropan-2-yl)carbamoyl)oxy)octyl 2-hexyldecanoate was synthesized according to representative synthetic scheme 2 and the corresponding methods. ¹H NMR (400 MHz, CDCl₃): δ 0.88 (9H, m), 1.26 (58H, m), 1.48-1.71 (12H, m), 2.31 (1H, m), 3.92-3.99 (2H, m), 4.06 (4H, m), 4.18 (2H, t, J=6.83 Hz), 4.34-4.46 (1H, m), 4.57-4.74 (1H, m), 5.49-5.62 (1H, m). MS [ESI]: m/z: [M+H] calc. 768.7 obs. 768.7.

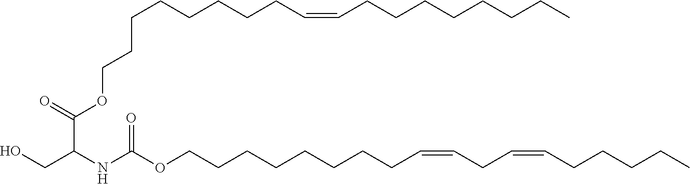 [SMILES: CCCCC/C=C\C/C=C\CCCCCCCCOC(=O)NC(CO)C(=O)OCCCCCCCC/C=C\CCCCCCCC]

(Z)-octadec-9-en-1-yl ((((9Z,12Z)-octadeca-9,12-dien-1-yl)oxy)carbonyl)serinate was synthesized according to representative synthetic scheme 2 and the corresponding methods. ¹H NMR (400 MHz, CDCl₃): δ 0.88 (6H, m), 1.30 (38H, m), 1.62 (4H, m), 2.04 (8H, m), 2.77 (2H, t, J=6.53 Hz), 3.95 (2H, m), 4.07 (2H, t, J=6.85 Hz), 4.18 (2H, t, J=6.96 Hz), 4.40 (1H, m), 5.36 (6H, m), 5.56 (1H, m). MS [ESI]: m/z: [M+H] calc. 648.6 obs. 648.5.

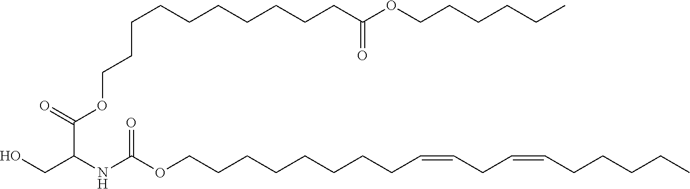 [SMILES: CCCCC/C=C\C/C=C\CCCCCCCCOC(=O)NC(CO)C(=O)OCCCCCCCCCCC(=O)OCCCCCC]

hexyl 11-((((((9Z,12Z)-octadeca-9,12-dien-1-yl)oxy)carbonyl)seryl)oxy)undecanoate was synthesized according to representative synthetic scheme 2 and the corresponding methods. ¹H NMR (400 MHz, CDCl₃): δ 0.89 (6H, t, J=6.98 Hz), 1.30 (34H, m), 1.62 (8H, m), 2.05 (4H, q, J=7.22 Hz), 2.28 (2H, t, J=7.86 Hz), 2.77 (2H, t, J=6.21 Hz), 3.95 (2H, m), 4.06 (4H, q, J=6.69 Hz), 4.18 (2H, td, J=6.88, 1.88 Hz), 4.41 (1H, m), 5.35 (4H, m), 5.57 (1H, m). MS [ESI]: m/z: [M+H] calc. 666.5 obs. 666.6.

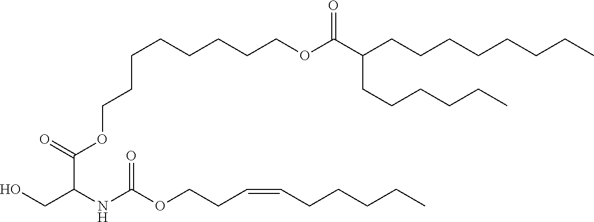 [SMILES: CCCCC/C=C\CCOC(=O)NC(CO)C(=O)OCCCCCCCCOC(=O)C(CCCCCC)CCCCCCCC]

(Z)-8-((((non-3-en-1-yloxy)carbonyl)seryl)oxy)octyl 2-hexyldecanoate was synthesized according to representative synthetic scheme 2 and the corresponding methods. ¹H NMR (400 MHz, CDCl₃): δ 0.88 (9H, m), 1.25 (36H, m), 1.50-1.72 (6H, m), 2.03 (2H, q, J=7.27 Hz), 2.23-2.34 (1H, m), 2.38 (2H, q, J=7.29 Hz), 3.95 (2H, m), 4.07 (4H, q, J=6.89 Hz), 4.17 (2H, m), 4.41 (1H, m), 5.42 (1H, m), 5.55-5.62 (1H, m). MS [ESI]: m/z: [M+H] calc. 640.5 obs. 640.6.

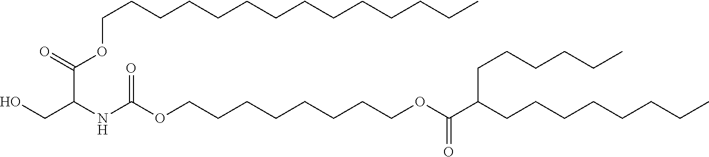 [SMILES: CCCCCCCCCCCCCCOC(=O)C(CO)NC(=O)OCCCCCCCCOC(=O)C(CCCCCC)CCCCCCCC]

8-(((1-(hexadecyloxy)-3-hydroxy-1-oxopropan-2-yl)carbamoyl)oxy)octyl 2-hexyldecanoate was synthesized according to representative synthetic scheme 2 and the corresponding methods. ¹H NMR (400 MHz, CDCl₃): δ 0.87 (9H, m), 1.26 (54H, m), 1.62 (10H, m), 2.31 (1H, m), 3.96 (2H, m), 4.07 (4H, t, J=6.62 Hz), 4.18 (2H, t, J=7.00 Hz), 4.35-4.46 (1H, m), 5.51-5.62 (1H, m). MS [ESI]: m/z: [M+H] calc. 740.6 obs.

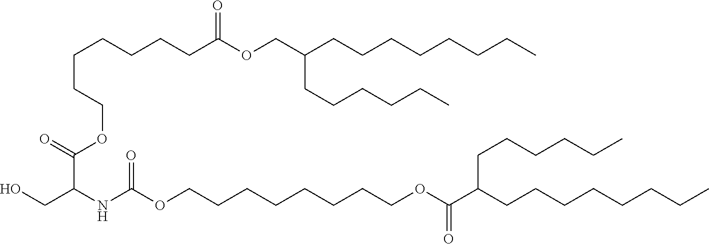 [SMILES: CCCCCCCCC(CCCCCC)COC(=O)CCCCCCCOC(=O)C(CO)NC(=O)OCCCCCCCCOC(=O)C(CCCCCC)CCCCCCCC]

25-hexyl-12-(hydroxymethyl)-10,13,22-trioxo-9,14,23-trioxa-11-azatritriacontyl 2-hexyldecanoate was synthesized according to representative synthetic scheme 2 and the corresponding methods. ¹H NMR (400 MHz, CDCl₃): δ 0.87 (12H, m), 1.26 (58H, m), 1.57 (13H, m), 2.29 (3H, m), 3.96 (4H, d, J=5.93 Hz), 4.02-4.10 (4H, m), 4.18 (2H, m), 4.41 (1H, m), 5.57 (1H, m). MS [ESI]: m/z: [M+H] calc. 882.7 obs. 882.8.

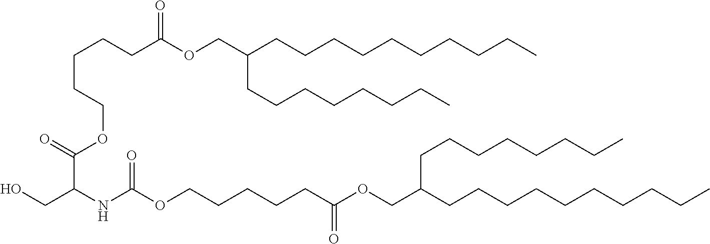 [SMILES: CCCCCCCCCCC(CCCCCCCC)COC(=O)CCCCCOC(=O)NC(CO)C(=O)OCCCCCC(=O)OCC(CCCCCCCC)CCCCCCCCCC]

2-octyldodecyl 6-(((3-hydroxy-1-((6-((2-octyldodecyl)oxy)-6-oxohexyl)oxy)-1-oxopropan-2-yl)carbamoyl)oxy)hexanoate was synthesized according to representative synthetic scheme 2 and the corresponding methods. ¹H NMR (400 MHz, CDCl₃): δ 0.88 (12H, t, J=6.66 Hz), 1.26 (60H, m), 1.40 (4H, m), 1.63 (14H, m), 2.31 (4H, t, J=7.34 Hz), 2.52 (1H, m), 3.96 (6H, d, J=5.81 Hz), 4.09 (4H, m), 4.40 (1H, m), 5.59 (1H, d, J=8.00 Hz). MS [ESI]: m/z: [M+H] calc. 938.8 obs. 938.8.

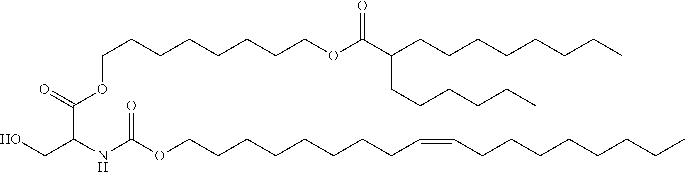 [SMILES: CCCCCCCC/C=C\CCCCCCCCOC(=O)NC(CO)C(=O)OCCCCCCCCOC(=O)C(CCCCCC)CCCCCCCC]

(Z)-8-((((octadec-9-en-1-yloxy)carbonyl)seryl)oxy)octyl 2-hexyldecanoate was synthesized according to representative synthetic scheme 2 and the corresponding methods. ¹H NMR (400 MHz, CDCl₃): δ 0.87 (9H, m), 1.25 (50H, m), 1.62 (10H, m), 2.04 (4H, m), 2.30 (1H, m), 3.95 (2H, m), 4.06 (6H, m), 4.41 (1H, m), 5.29 (1H, s), 5.34 (2H, m), 5.58 (1H, m). MS [ESI]: m/z: [M+H] calc. 766.6 obs. 766.7.

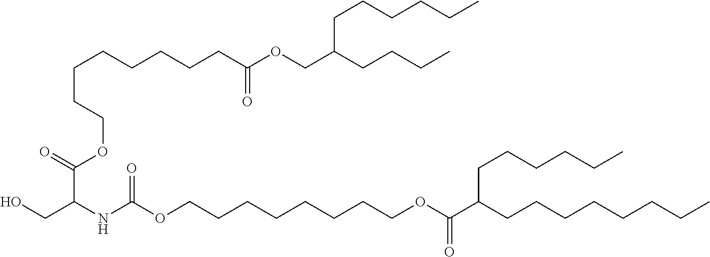 [SMILES: CCCCCCCCC(CCCCCC)C(=O)OCCCCCCCCOC(=O)NC(CO)C(=O)OCCCCCCCCC(=O)OCC(CCCC)CCCCCC]

26-butyl-12-(hydroxymethyl)-10,13,23-trioxo-9,14,24-trioxa-11-azadotriacontyl 2-hexyldecanoate was synthesized according to representative synthetic scheme 2 and the corresponding methods. 4H NMR (400 MHz, CDCl₃): δ 0.87 (12H, m), 1.25 (54H, m), 1.61 (11H, m), 2.29 (3H, t, J=7.45 Hz), 3.96 (4H, m), 4.07 (4H, m), 4.18 (2H, m), 4.36-4.46 (1H, m), 5.57 (1H, d, J=5.76 Hz). MS [ESI]: m/z: [M+H] calc. 840.7 obs. 840.6.

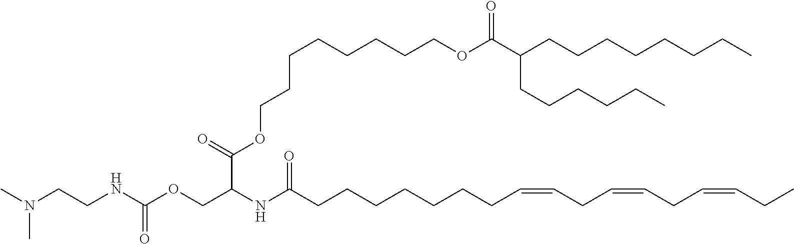 [SMILES: CC/C=C\C/C=C\C/C=C\CCCCCCCC(=O)NC(COC(=O)NCCN(C)C)C(=O)OCCCCCCCCOC(=O)C(CCCCCC)CCCCCCCC]

2-methyl-9-((9Z,12Z,15Z)-octadeca-9,12,15-trienamido)-6,10-dioxo-7,11-dioxa-2,5-diazanonadecan-19-yl 2-hexyldecanoate was synthesized according to method 4. MS [ESI]: m/z: [M+H]. calc. 847.3 obs. 848.2; ¹H NMR (400 MHz, CDCl₃): δ 0.88 (9H, m), 1.26 (38H, m), 1.61 (12H, m), 1.78 (4H, m), 2.05 (4H, m), 2.26 (4H, m), 2.50 (2H, d, J=0.60 Hz), 2.57 (2H, s), 2.77 (2H, m), 3.27 (1H, d, J=1.68 Hz), 4.15 (2H, t, J=6.76 Hz), 4.31-4.37 (1H, m), 4.41-4.47 (1H, m), 4.85 (2H, m), 5.30 (5H, m), 6.37-6.42 (1H, m).

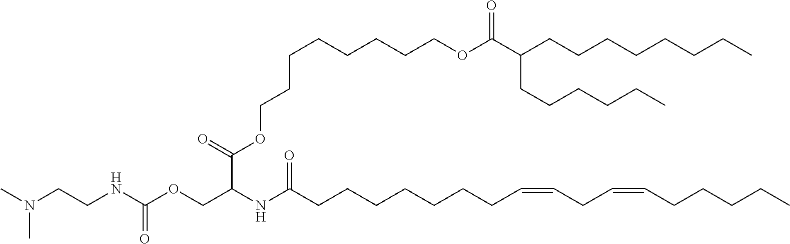 [SMILES: CCCCC/C=C\C/C=C\CCCCCCCC(=O)NC(COC(=O)NCCN(C)C)C(=O)OCCCCCCCCOC(=O)C(CCCCCC)CCCCCCCC]

2-methyl-9-((9Z,12Z)-octadeca-9,12-dienamido)-6,10-dioxo-7,11-dioxa-2,5-diazanonadecan-19-yl 2-hexyldecanoate was synthesized according to method 4.

MS [ESI]: m/z: [M+H]. calc. 849.3 obs. 849.8; ¹H NMR (400 MHz, CDCl₃): δ 0.87 (9H, m), 1.25 (43H, m), 1.63 (11H, m), 2.05 (4H, m), 2.21-2.27 (7H, 2.22 (m), 2.24 (s)), 2.31 (1H, s), 2.41 (2H, m), 2.77 (2H, t, J=6.04 Hz), 3.25 (1H, m), 4.06 (2H, t, J=6.68 Hz), 4.15 (2H, t, J=6.77 Hz), 4.30-4.37 (1H, m), 4.42-4.48 (1H, m), 4.77-4.83 (1H, m) 5.35 (4H, m), 6.40 (1H, d, J=8.11 Hz).

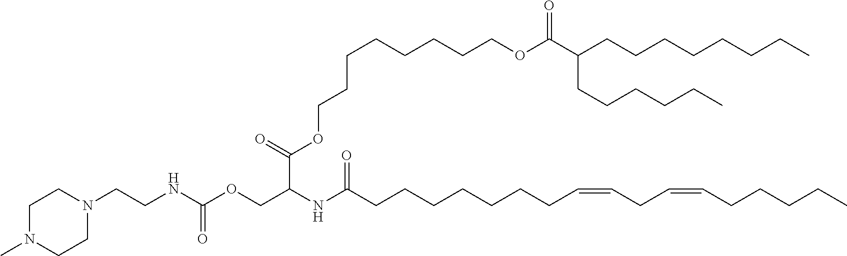 [SMILES: CCCCC/C=C\C/C=C\CCCCCCCC(=O)NC(COC(=O)NCCN1CCN(C)CC1)C(=O)OCCCCCCCCOC(=O)C(CCCCCC)CCCCCCCC]

8-((O-((2-(4-methylpiperazin-1-yl)ethyl)carbamoyl)-N-((9Z,12Z)-octadeca-9,12-dienoyl)seryl)oxy)octyl 2-hexyldecanoate was synthesized according to method 4. MS [ESI]: m/z: [M+H]. calc. 904.4 obs. 904.8; ¹H NMR (400 MHz, CDCl₃): δ 0.89 (9H, m), 1.31 (42H, m), 1.45 (1H, s), 1.61 (11H, m), 2.06 (4H, m), 2.31 (6H, m), 2.48 (7H, m), 2.79 (2H, t, J=6.51 Hz), 3.28 (1H, m), 4.08 (2H, t, J=6.67 Hz), 4.13-4.20 (2H, m), 4.44-4.49 (1H, m), 5.36 (5H, m), 6.40 (1H, d, J=7.60 Hz), 4.82 (1H, m), 4.32-4.40 (1H, m), 5.18-5.26 (1H, m).

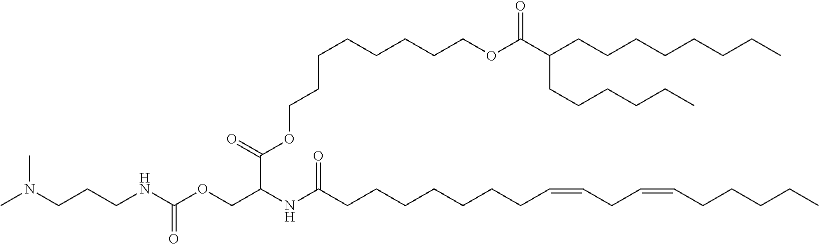 [SMILES: CCCCC/C=C\C/C=C\CCCCCCCC(=O)NC(COC(=O)NCCCN(C)C)C(=O)OCCCCCCCCOC(=O)C(CCCCCC)CCCCCCCC]

2-methyl-10-((9Z,12Z)-octadeca-9,12-dienamido)-7,11-dioxo-8,12-dioxa-2,6-diazaicosan-20-yl 2-hexyldecanoate was synthesized according to method 4. MS [ESI]: m/z: [M+H]. calc. 863.3 obs. 863.7; ¹H NMR (400 MHz, CDCl₃): δ 0.87 (9H, m), 1.29 (42H, m), 1.59 (13H, m), 2.05 (3H, m), 2.23 (7H, m), 2.26-2.44 (4H, m), 2.77 (2H, t, J=6.53 Hz), 3.24 (2H, m), 4.06 (2H, t, J=6.69 Hz), 4.14 (1H, t, J=6.67 Hz), 4.28-4.37 (1H, m), 4.40-4.49 (1H, m), 4.73-4.82 (2H, m), 5.34 (4H, m), 5.71-5.78 (1H, m), 6.38-6.47 (1H, m).

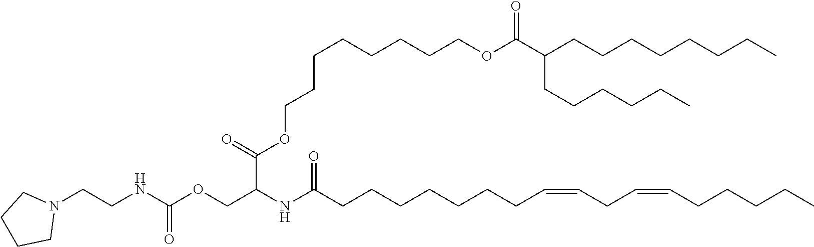 [SMILES: CCCCC/C=C\C/C=C\CCCCCCCC(=O)NC(COC(=O)NCCN1CCCC1)C(=O)OCCCCCCCCOC(=O)C(CCCCCC)CCCCCCCC]

8-((N-((9Z,12Z)-octadeca-9,12-dienoyl)-O-((2-pyrrolidin-1-yl)ethyl)carbamoyl)seryl)oxy)octyl 2-hexyldecanoate was synthesized according to method 4. MS [ESI]: m/z: [M+H]. calc. 875.4 obs. 874.8; ¹H NMR (400 MHz, CDCl₃): δ 0.87 (9H, m), 1.25 (44H, m), 1.61 (12H, m), 1.78 (3H, m), 2.04 (4H, m), 2.24 (2H, m), 2.51 (4H, s), 2.58 (1H, m), 2.77 (2H, t, J=6.11 Hz), 3.27 (2H, m) 4.06 (2H, t, J=6.68 Hz), 4.15 (2H, t, J=6.76 Hz), 4.31-4.37 (1H, m), 4.41-4.47 (1H, m), 4.80 (1H, dt, J=7.69, 3.76 Hz), 5.34 (4H, s), 6.31-6.45 (1H, m).

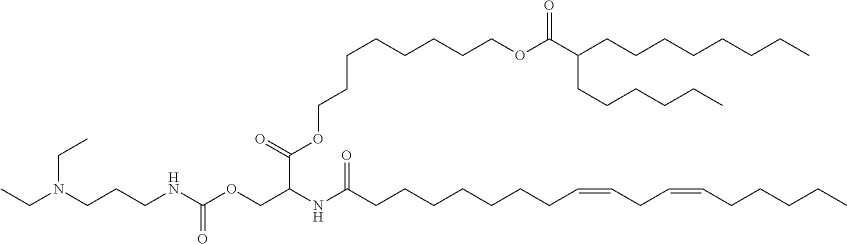 [SMILES: CCCCC/C=C\C/C=C\CCCCCCCC(=O)NC(COC(=O)NCCCN(CC)CC)C(=O)OCCCCCCCCOC(=O)C(CCCCCC)CCCCCCCC]

3-ethyl-11-((9Z,12Z)-octadeca-9,12-dienamido)-8,12-dioxo-9,13-dioxa-3,7-diazahenicosan-21-yl 2-hexyldecanoate was synthesized according to method 4. MS [ESI]: m/z: [M+H]. calc. 891.4 obs. 891.8; ¹H NMR (400 MHz, CDCl₃): δ 0.87 (9H, m), 1.02 (6H, t, J=7.12 Hz), 1.25 (46H, m), 1.55 (5H, s), 1.62 (6H, m), 2.05 (4H, q, J=8.19 Hz), 2.23 (2H, t, J=7.66 Hz), 2.26-2.35 (1H, m), 2.48 (4H, m), 2.77 (2H, t, J=5.99 Hz), 3.24 (1H, m), 4.06 (2H, t, J=6.66 Hz), 4.13 (2H, t, J=6.76 Hz), 4.31-4.36 (1H, m), 4.38-4.45 (1H, m), 4.73-4.80 (1H, m), 5.34 (4H, m), 6.40 (1H, m).

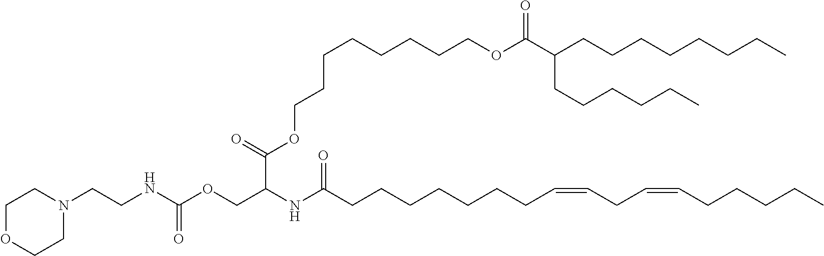 [SMILES: CCCCC/C=C\C/C=C\CCCCCCCC(=O)NC(COC(=O)NCCN1CCOCC1)C(=O)OCCCCCCCCOC(=O)C(CCCCCC)CCCCCCCC]

8-((O-((2-morpholinoethyl)carbamoyl)-N-((9Z,12Z)-octadeca-9,12-dienoyl)seryl)oxy)octyl 2-hexyldecanoate was synthesized according to method 4. MS [ESI]: m/z: [M+H]. calc. 891.3 obs. 891.7; ¹H NMR (400 MHz, CDCl₃): δ 0.88 (9H, m), 1.32 (41H, m), 1.48-1.78 (12H, 1.63 (m), 2.04 (4H, m), 2.24 (2H, t, J=7.65 Hz), 2.28-2.34 (1H, m), 2.45 (6H, m), 2.77 (2H, m), 3.27 (1H, m), 3.70 (4H, m), 4.06 (2H, t, J=6.69 Hz), 4.15 (2H, td, J=6.72, 4.34 Hz), 4.31-4.38 (1H, m), 4.43 (1H, d, J=4.27 Hz), 4.76-4.84 (1H, m), 5.15-5.23 (1H, m), 5.34 (4H, m), 6.33-6.39 (1H, m).

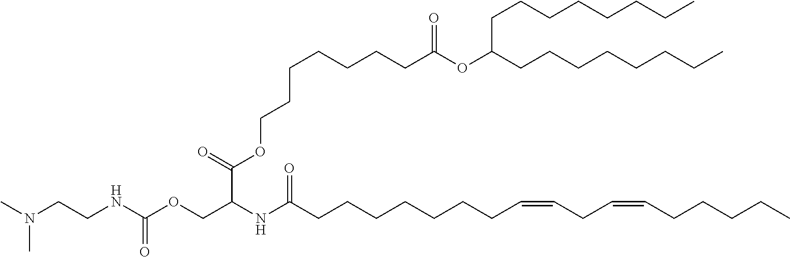 [SMILES: CCCCC/C=C\C/C=C\CCCCCCCC(=O)NC(COC(=O)NCCN(C)C)C(=O)OCCCCCCCC(=O)OC(CCCCCCCC)CCCCCCCC]

heptadecan-9-yl 8-((O-((2-(dimethylamino)ethyl)carbamoyl)-N-((9Z,12Z)-octadeca-9,12-dienoyl)seryl)oxy)octanoate was synthesized according to method 4. MS [ESI]: m/z: [M+H]. calc. 863.3 obs. 863.9; ¹H NMR (500 MHz, CDCl₃): δ 0.88 (9H, m), 1.27 (45H, m), 1.50 (2H, m), 1.60 (7H, m), 2.05 (2H, m), 2.22 (10H, m), 2.39 (2H, t, J=5.63 Hz), 2.75-2.79 (2H, m), 3.18-3.30 (3H, m). 4.15 (2H, t, J=6.77 Hz), 4.30-4.37 (1H, m), 4.42-4.47 (1H, m), 4.77-4.82 (1H, m), 4.84-4.89 (2H, m), 5.24-5.28 (1H, m), 5.35 (4H), 6.36-6.40 (1H, m).

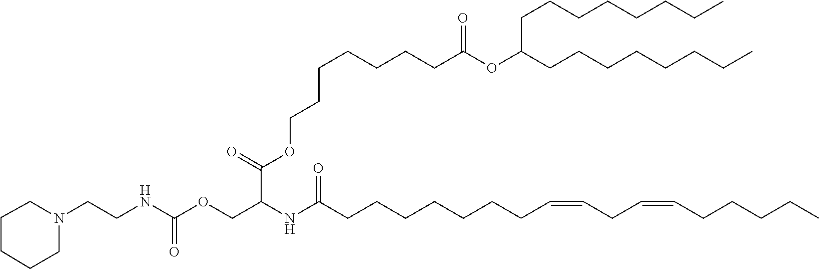 [SMILES: CCCCC/C=C\C/C=C\CCCCCCCC(=O)NC(COC(=O)NCCN1CCCCC1)C(=O)OCCCCCCCC(=O)OC(CCCCCCCC)CCCCCCCC]

heptadecan-9-yl 8-((N-((9Z,12Z)-octadeca-9,12-dienoyl)-O-((2-(piperidin-1-yl)ethyl)carbamoyl)seryl)oxy)octanoate was synthesized according to method 4. MS [ESI]: m/z: [M+H]. calc. 903.4 obs. 902.9; ¹H NMR (500 MHz, CDCl₃): δ 0.88 (9H, m), 1.26 (45H, m), 1.59 (18H, m), 2.04 (4H, m), 2.27 (4H, m), 2.37 (4H, m), 2.77 (2H, t, J=6.82 Hz), 3.24 (1H, m), 4.15 (2H, t, J=6.60 Hz), 4.31-4.37 (1H, m), 4.41-4.47 (1H, m), 4.76-4.82 (2H, m), 4.86 (1H, m), 5.35 (4H, m), 6.42 (1H, d, J=7.80 Hz).

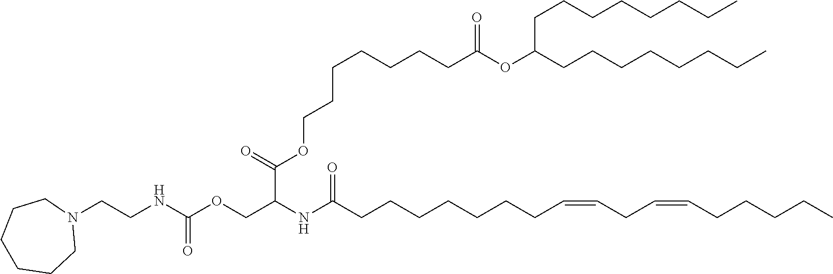 [SMILES: CCCCC/C=C\C/C=C\CCCCCCCC(=O)NC(COC(=O)NCCN1CCCCCC1)C(=O)OCCCCCCCC(=O)OC(CCCCCCCC)CCCCCCCC]

heptadecan-9-yl 8-((O-((2-(azepan-1-yl)ethyl)carbamoyl)-N-((9Z,12Z)-octadeca-9,12-dienoyl)seryl)oxy)octanoate was synthesized according to method 4. MS [ESI]: m/z: [M+H]. calc. 917.4 obs. 916.9; ¹H NMR (500 MHz, CDCl₃): δ 0.88 (9H, m), 1.26 (50H, m), 1.50 (4H, m), 1.61 (14H, m), 2.05 (2H, m), 2.27 (3H, m), 2.61 (1H, m), 2.77 (2H, t, J=6.77 Hz), 3.16-3.23 (2H, m), 4.15 (2H, t, J=6.77 Hz), 4.31-4.37 (1H, m), 4.42-4.47 (1H, m) 4.77-4.82 (1H, m), 4.83-4.88 (2H, m), 5.35 (5H, s) 6.39-6.44 (1H m).

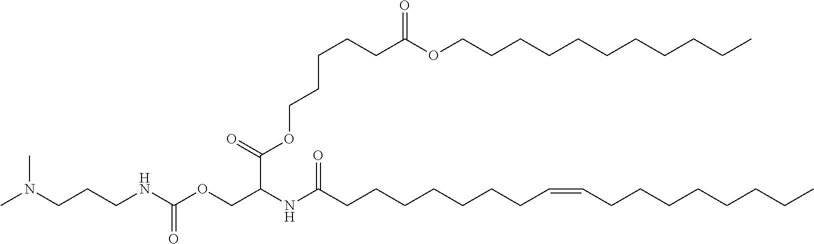 [SMILES: CCCCCCCC/C=C\CCCCCCCC(=O)NC(COC(=O)NCCCN(C)C)C(=O)OCCCCCC(=O)OCCCCCCCCCCC]

undecyl 6-((O-((3-(dimethylamino)propyl)carbamoyl)-N-oleoylseryl)oxy)hexanoate was synthesized according to method 4. MS [ESI]: m/z: [M+H]. calc. 767.1 obs. 767.9; ¹H NMR (400 MHz, CDCl₃): δ 0.88 (6H, t, J=6.81 Hz), 1.26 (37H, m), 1.58-1.68 (11H, m), 2.00 (4H, m), 2.21 (13H, m), 2.61-2.66 (1H, m), 3.23 (1H, q, J=6.49 Hz), 4.05 (2H, t, J=6.78 Hz), 4.15 (1H, m), 5.31-5.36 (2H, m), 4.31-4.36 (1H, m), 4.38-4.44 (1H, m), 4.74-4.80 (1H, m), 5.74-5.83 (1H, m), 3.30-3.36 (1H, m).

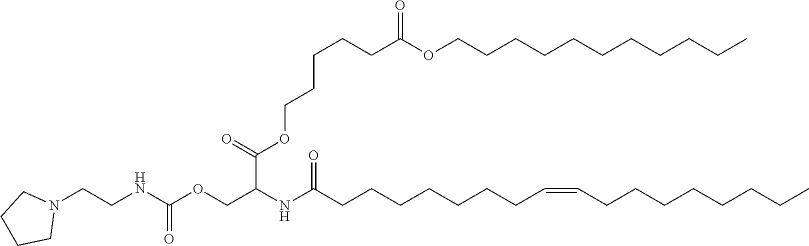 [SMILES: CCCCCCCC/C=C\CCCCCCCC(=O)NC(COC(=O)NCCN1CCCC1)C(=O)OCCCCCC(=O)OCCCCCCCCCCC]

undecyl 6-((N-oleoyl-O-((2-(pyrrolidin-1-yl)ethyl)carbamoyl)seryl)oxy)hexanoate was synthesized according to method 4. MS [ESI]: m/z: [M+H]. calc. 779.1 obs. 778; ¹H NMR (400 MHz, CDCl₃): δ 0.90 (6H, t, J=6.80 Hz), 1.28 (45H, m), 2.02 (4H, q, J=5.38 Hz), 2.23-2.27 (1H, m), 2.32 (2H, t, J=7.44 Hz), 2.52 (3H, m), 2.57-2.62 (2H, m), 4.07 (2H, t, J=6.79 Hz), 5.36 (2H, m), 4.33-4.39 (1H, m), 4.43-4.50 (1H, m), 4.59-4.64 (1H, m), 4.78-4.84 (1H, m), 1.63-1.68 (6H, m), 1.76-1.82 (4H, m), 4.13-4.21 (2H, m).

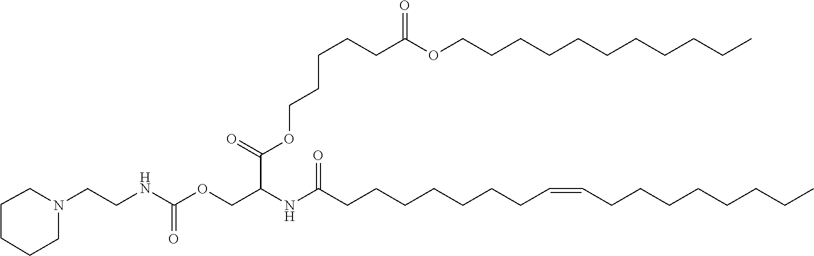 [SMILES: CCCCCCCC/C=C\CCCCCCCC(=O)NC(COC(=O)NCCN1CCCCC1)C(=O)OCCCCCC(=O)OCCCCCCCCCCC]

undecyl 6-((N-oleoyl-O-((2-(piperidin-1-yl)ethyl)carbamoyl)seryl)oxy)hexanoate was synthesized according to method 4. MS [ESI]: m/z: [M+H]. calc. 793.2 obs. 792.6.

¹H NMR (400 MHz, CDCl₃): δ 0.87-0.93 (6H, 0.90 (t, J=6.81 Hz), 1.28 (42H, m), 1.58 (12H, m), 1.99-2.05 (4H, m), 2.23-2.44 (10H), 3.22-3.31 (2H, m), 4.07 (2H, t, J=6.78 Hz), 4.19 (1H, m), 4.33-4.39 (1H, m), 4.43-4.49 (1H, m), 4.80-4.84 (1H, m), 5.34-5.38 (2H, m), 6.43-6.48 (1H, m).

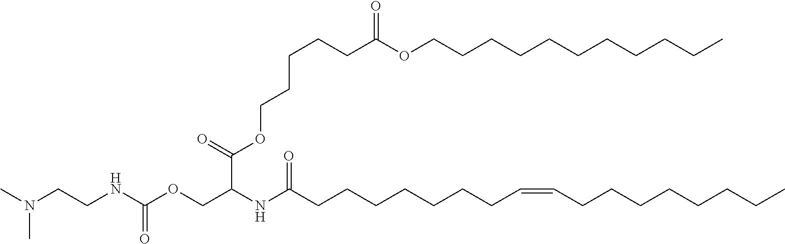 [SMILES: CCCCCCCC/C=C\CCCCCCCC(=O)NC(COC(=O)NCCN(C)C)C(=O)OCCCCCC(=O)OCCCCCCCCCCC]

undecyl 6-((O-((2-(dimethylamino)ethyl)carbamoyl)-N-oleoylseryl)oxy)hexanoate was synthesized according to method 4. MS [ESI]: m/z: [M+H]. calc. 753.1 obs. 752.7; ¹H NMR (500 MHz, CDCl₃): δ 0.86-0.90 (6H, 0.88 (t, J=6.74 Hz), 1.24 (43H, m), 1.60-1.66 (6H, m), 2.01 (2H, m), 2.21-2.24 (7H, 2.23 m), 2.30 (2H, m), 2.38-2.41 (2H, m), 3.22-3.26 (2H, m), 4.05 (3H, t, J=6.78 Hz), 4.14-4.17 (2H, m), 4.33-4.36 (1H, m), 4.42-4.45 (1H, m), 4.78-4.81 (1H, m), 5.33-5.35 (2H, m), 6.41-6.44 (1H, m).

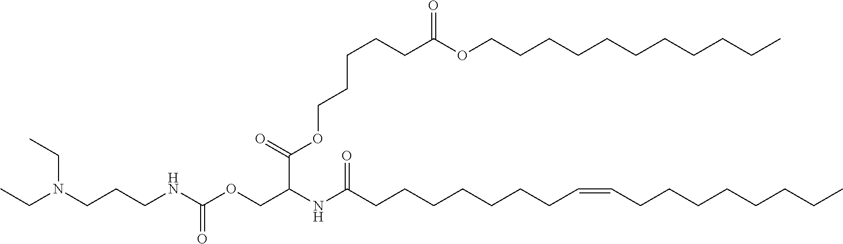 [SMILES: CCCCCCCC/C=C\CCCCCCCC(=O)NC(COC(=O)NCCCN(CC)CC)C(=O)OCCCCCC(=O)OCCCCCCCCCCC]

undecyl 6-((O-((3-(diethylamino)propyl)carbamoyl)-N-oleoylseryl)oxy)hexanoate was synthesized according to method 4. MS [ESI]: m/z: [M+H]. calc. 795.2 obs. 795.8;

¹H NMR (500 MHz, CDCl₃): δ 0.88 (6H, t, J=6.96 Hz), 1.02 (5H, t, J=7.07 Hz), 1.26 (39H, m), 1.59-1.68 (10H, m), 1.99-2.03 (4H, m), 2.23 (2H, dd, J=8.44, 6.73 Hz), 2.30 (2H, t, J=7.35 Hz), 2.47-2.51 (4H, m), 3.23-3.26 (3H, m), 4.05 (3H, t, J=6.76 Hz), 4.13-4.17 (3H, m), 4.33-4.35 (1H, m), 4.40-4.42 (1H, m), 4.75-4.78 (1H, m), 5.32-5.36 (2H, m), 6.44-6.47 (1H, m).

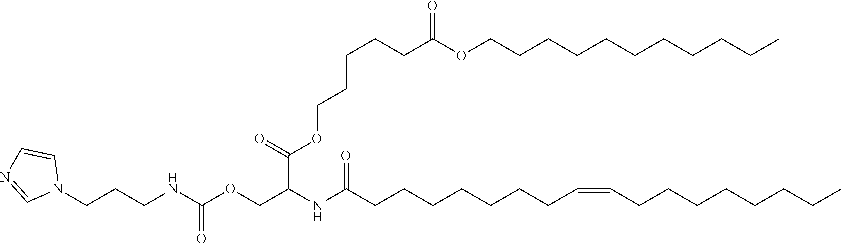 [SMILES: CCCCCCCC/C=C\CCCCCCCC(=O)NC(COC(=O)NCCCn1ccnc1)C(=O)OCCCCCC(=O)OCCCCCCCCCCC]

undecyl 6-((O-((3-(1H-imidazol-1-yl)propyl)carbamoyl)-N-oleoylseryl)oxy)hexanoate was synthesized according to method 4. MS [ESI]: m/z: [M+H]. calc. 790.2 obs. 790.8;

¹H NMR (400 MHz, CDCl₃): δ 0.88 (6H, t, J=6.81 Hz), 1.26 (38H, m), 1.58-1.74 (8H, m), 1.98 (6H, m), 2.29 (4H, m), 3.15-3.22 (2H, m), 4.03 (4H, dt, J=12.32, 6.79 Hz), 4.12-4.23 (2H, m), 4.39 (2H, m), 4.77-4.83 (1H, m), 5.01-5.08 (1H, m), 5.31-5.37 (2H, m), 6.58-6.65 (1H, m), 6.94 (1H, s), 7.07 (1H, s), 7.50 (1H, s).

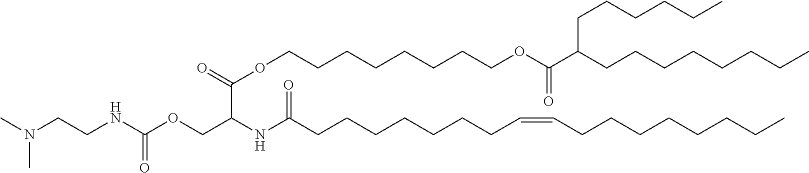 [SMILES: CCCCCCCC/C=C\CCCCCCCC(=O)NC(COC(=O)NCCN(C)C)C(=O)OCCCCCCCCOC(=O)C(CCCCCC)CCCCCCCC]

2-Methyl-9-oleamido-6,10-dioxo-7,11-dioxa-2,5-diazanonadecan-19-yl 2-hexyldecanoate was synthesized according to representative synthetic scheme 1 and the corresponding methods. ¹H NMR (400 MHz, CDCl₃): δ 0.87 (9H, m), 1.02 (6H, t, J=7.13 Hz), 1.25 (48H, m), 1.60 (12H, m), 2.01 (4H, m), 2.23 (2H, t, J=8.06 Hz), 2.31 (1H, m), 2.47 (6H, m), 3.24 (2H, m), 4.06 (2H, t, J=6.66 Hz), 4.13 (2H, t, J=6.85 Hz), 4.33 (1H, dd, J=11.37, 3.26 Hz), 4.42 (1H, dd, J=11.37, 4.41 Hz), 4.77 (1H, m), 5.34 (2H, m), 6.40 (2H, m). MS [ESI]: m/z: [M+H] calc. 8507 obs. 850.5.

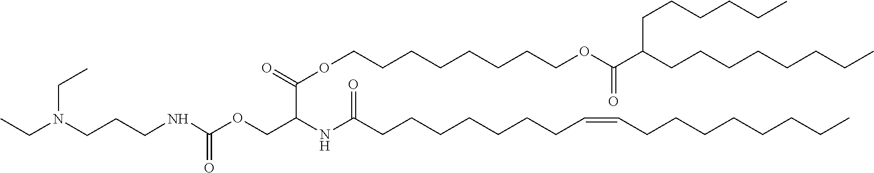 [SMILES: CCCCCCCC/C=C\CCCCCCCC(=O)NC(COC(=O)NCCCN(CC)CC)C(=O)OCCCCCCCCOC(=O)C(CCCCCC)CCCCCCCC]

3-Ethyl-11-oleamido-8,12-dioxo-9,13-dioxa-3,7-diazahenicosan-21-yl 2-hexyldecanoate was synthesized according to representative synthetic scheme 1 and the corresponding methods. ¹H NMR (400 MHz, CDCl₃): δ 0.87 (9H, m), 1.02 (6H, t, J=7.13 Hz), 1.25 (48H, m), 1.60 (12H, m), 2.01 (4H, m), 2.23 (2H, t, J=8.06 Hz), 2.31 (1H, m), 2.47 (6H, m), 3.24 (2H, m), 4.06 (2H, t, J=6.66 Hz), 4.13 (2H, t, J=6.85 Hz), 4.33 (111, dd, J=11.37, 3.26 Hz), 4.42 (1H, dd, J=11.37, 4.41 Hz), 4.77 (111, m), 5.34 (2H, m), 6.40 (2H, m). MS [ESI]: m/z: [M+H] calc. 892.8 obs. 892.6.

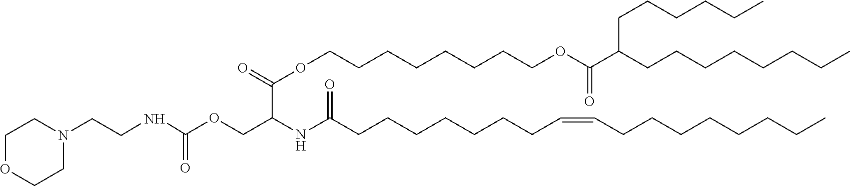 [SMILES: CCCCCCCC/C=C\CCCCCCCC(=O)NC(COC(=O)NCCN1CCOCC1)C(=O)OCCCCCCCCOC(=O)C(CCCCCC)CCCCCCCC]

8-((O-((2-Morpholinoethyl)carbamoyl)-N-oleoylseryl)oxy)octyl 2-hexyldecanoate was synthesized according to representative synthetic scheme 1 and the corresponding methods. ¹H NMR (400 MHz, CDCl₃): δ 0.87 (9H, m), 1.25 (48H, m), 1.62 (10H, m), 2.01 (4H, m), 2.24 (2H, t, J=7.90 Hz), 2.30 (1H, m), 2.44 (6H, m), 3.26 (2H, m), 3.70 (4H, m), 4.06 (2H, t, J=6.66 Hz), 4.15 (2H, m), 4.35 (1H, dd, J=11.34, 2.98 Hz), 4.44 (1H, dd, J=11.34, 4.40 Hz), 4.80 (1H, m), 5.20 (1H, m), 5.34 (2H, m), 6.36 (1H, d, J=7.67 Hz). MS [ESI]: m/z: [M+H] calc. 892.7 obs. 892.5.

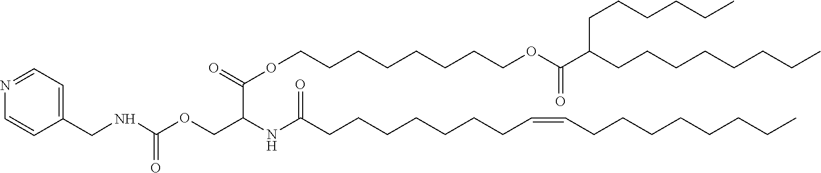 [SMILES: CCCCCCCC/C=C\CCCCCCCC(=O)NC(COC(=O)NCc1ccncc1)C(=O)OCCCCCCCCOC(=O)C(CCCCCC)CCCCCCCC]

8-((N-Oleoyl-O-((pyridin-4-ylmethyl)carbamoyl)seryl)oxy)octyl 2-hexyldecanoate was synthesized according to representative synthetic scheme 1 and the corresponding methods. ¹H NMR (400 MHz, CDCl₃): δ 0.87 (9H, m), 1.25 (48H, m), 1.59 (10H, m), 2.00 (4H, m), 2.22 (2H, t, J=7.65 Hz), 2.30 (1H, m), 4.05 (2H, t, J=6.68 Hz), 4.14 (2H, m), 4.37 (2H, d, J=6.46 Hz), 4.42 (1H, dd, J=11.58, 3.10 Hz), 4.49 (1H, dd, J=11.58, 4.31 Hz), 4.83 (1H, m), 5.21 (1H, t, J=6.37 Hz), 5.34 (2H, m), 6.30 (1H, d, J=7.69 Hz), 7.19 (2H, d, J=5.97 Hz), 8.57 (2H, d, J=5.97 Hz). MS [ESI]: m/z: [M+H] calc. 870.7 obs. 870.5.

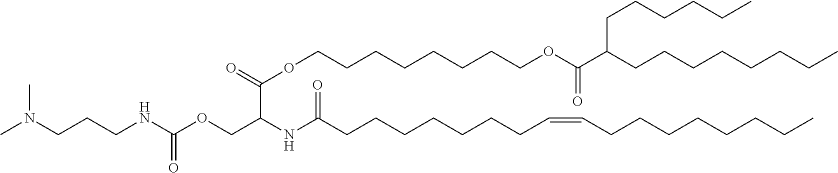 [SMILES: CCCCCCCC/C=C\CCCCCCCC(=O)NC(COC(=O)NCCCN(C)C)C(=O)OCCCCCCCCOC(=O)C(CCCCCC)CCCCCCCC]

2-Methyl-10-oleamido-7,11-dioxo-8,12-dioxa-2,6-diazaicosan-20-yl 2-hexyldecanoate was synthesized according to representative synthetic scheme 1 and the corresponding methods. ¹H NMR (400 MHz, CDCl₃): δ 0.87 (9H, m), 1.25 (48H, m), 1.63 (12H, m), 2.00 (4H, m), 2.21 (11H, m), 3.23 (2H, m), 4.06 (2H, t, J=6.70 Hz), 4.14 (211, t, J=6.63 Hz), 4.33 (111, dd, J=11.47, 3.10 Hz), 4.42 (1H, dd, J=11.47, 4.54 Hz), 4.78 (111, m), 5.34 (2H, m), 5.74 (1H, m), 6.43 (1H, d, J=7.86 Hz). MS [ESI]: m/z: [M+H] calc. 864.7 obs. 864.6.

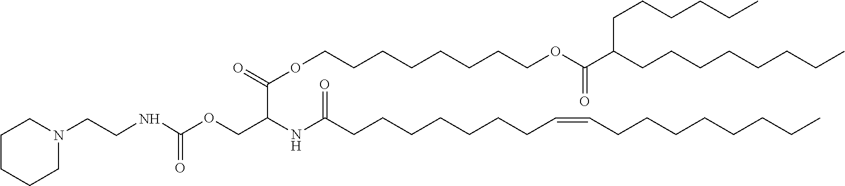 [SMILES: CCCCCCCC/C=C\CCCCCCCC(=O)NC(COC(=O)NCCN1CCCCC1)C(=O)OCCCCCCCCOC(=O)C(CCCCCC)CCCCCCCC]

8-((N-Oleoyl-O-((2-(piperidin-1-yl)ethyl)carbamoyl)seryl)oxy)octyl 2-hexyldecanoate was synthesized according to representative synthetic scheme 1 and the corresponding methods. ¹H NMR (400 MHz, CDCl₃): δ 0.87 (9H, m), 1.25 (50H, m), 1.58 (14H, m), 2.00 (4H, m), 2.32 (9H, m), 3.23 (2H, m), 4.06 (2H, t, J=6.66 Hz), 4.15 (2H, t, J=6.90 Hz), 4.34 (1H, dd, J=11.64, 2.95 Hz), 4.44 (1H, dd, J=11.64, 4.30 Hz), 4.79 (1H, m), 5.34 (3H, m), 6.41 (1H, d, J=7.85 Hz). MS [ESI]: m/z: [M+H] calc. 890.8 obs. 890.6.

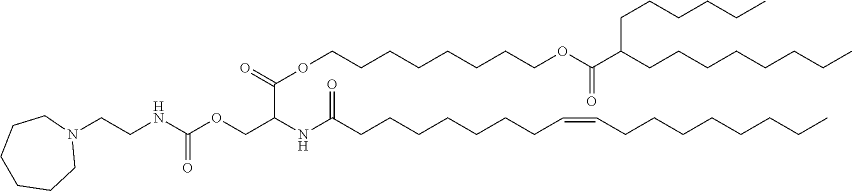 [SMILES: CCCCCCCC/C=C\CCCCCCCC(=O)NC(COC(=O)NCCN1CCCCCC1)C(=O)OCCCCCCCCOC(=O)C(CCCCCC)CCCCCCCC]

8-((O-((2-(Azepan-1-yl)ethyl)carbamoyl)-N-oleoylseryl)oxy)octyl 2-hexyldecanoate was synthesized according to representative synthetic scheme 1 and the corresponding methods. ¹H NMR (400 MHz, CDCl₃): δ 0.87 (911, m), 1.25 (48H, m), 1.59 (18H, m), 2.00 (411, m), 2.23 (3H, m), 2.61 (6H, m), 3.19 (2H, m), 4.05 (2H, t, J=6.73 Hz), 4.14 (2H, t, J=6.79 Hz), 4.33 (1H, dd, J=11.72, 2.68 Hz), 4.44 (1H, dd, J=11.72, 4.42 Hz), 4.79 (1H, m), 5.29 (311, m), 6.42 (1H, d, J=8.02 Hz). MS [ESI]: m/z: [M+H] calc. 904.8 obs. 904.6.

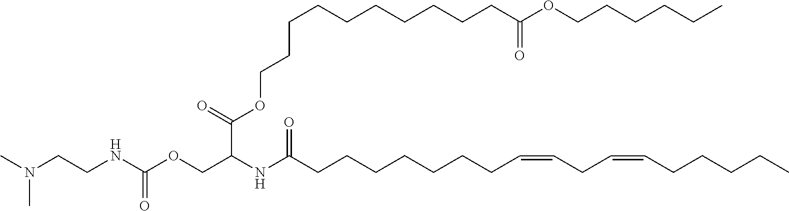 [SMILES: CCCCC/C=C\C/C=C\CCCCCCCC(=O)NC(COC(=O)NCCN(C)C)C(=O)OCCCCCCCCCCC(=O)OCCCCCC]

Hexyl 11-((O-((2-(dimethylamino)ethyl)carbamoyl)-N-((9Z,12Z)-octadeca-9,12-dienoyl)seryl)oxy)undecanoate was synthesized according to representative synthetic scheme 1 and the corresponding methods. ¹H NMR (400 MHz, CDCl₃): δ 0.89 (6H, t, J=6.99 Hz), 1.31 (32H, m), 1.64 (8H, m), 2.05 (4H, q, J=7.15 Hz), 2.22 (10H, m), 2.39 (2H, t, J=5.84 Hz), 2.77 (2H, t, J=6.04 Hz), 3.23 (2H, m), 4.05 (2H, t, J=6.75 Hz), 4.15 (2H, t, J=6.75 Hz), 4.34 (111, dd, J=11.52, 3.01 Hz), 4.44 (111, dd, J=11.52, 4.19 Hz), 4.80 (111, m), 5.24 (111, m), 5.34 (4H, m), 6.37 (1H, d, J=7.86 Hz). MS [ESI]: m/z: [M+H] calc. 750.6 obs. 750.6.

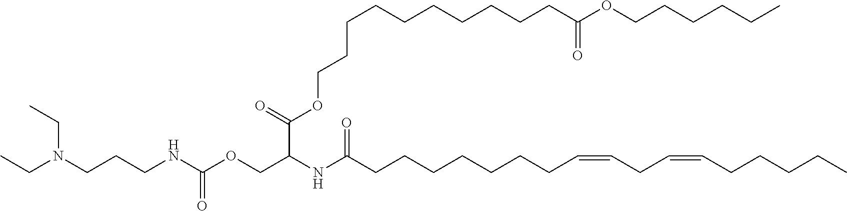 [SMILES: CCCCC/C=C\C/C=C\CCCCCCCC(=O)NC(COC(=O)NCCCN(CC)CC)C(=O)OCCCCCCCCCCC(=O)OCCCCCC]

Hexyl 11-((O-((3-(diethylamino)propyl)carbamoyl)-N-((9Z,12Z)-octadeca-9,12-dienoyl)seryl)oxy)undecanoate was synthesized according to representative synthetic scheme 1 and the corresponding methods. ¹H NMR (400 MHz, CDCl₃): δ 0.89 (6H, t, J=7.03 Hz), 1.02 (6H, t, J=7.13 Hz), 1.30 (32H, m), 1.62 (10H, m), 2.05 (4H, q, J=7.17 Hz), 2.23 (2H, t, J=7.77 Hz), 2.29 (2H, t, J=7.63 Hz), 2.49 (6H, m), 2.77 (2H, t, J=6.45 Hz), 3.24 (2H, m), 4.06 (2H, t, J=6.75 Hz), 4.13 (2H, t, J=6.55 Hz), 4.33 (1H, dd, J=11.50, 3.10 Hz), 4.41 (1H, dd, J=11.50, 4.55 Hz), 4.77 (1H, m), 5.34 (4H, m), 6.40 (2H, m). MS [ESI]: m/z: [M+H] calc. 792.6 obs. 792.7.

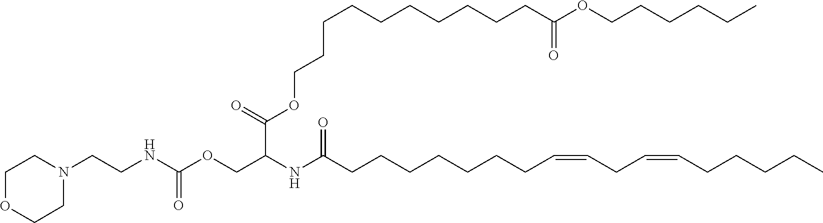 [SMILES: CCCCC/C=C\C/C=C\CCCCCCCC(=O)NC(COC(=O)NCCN1CCOCC1)C(=O)OCCCCCCCCCCC(=O)OCCCCCC]

Hexyl 11-((O-((2-morpholinoethyl)carbamoyl)-N-((9Z,12Z)-octadeca-9,12-dienoyl)seryl)oxy)undecanoate was synthesized according to representative synthetic scheme 1 and the corresponding methods. ¹H NMR (400 MHz, CDCl₃): δ 0.89 (6H, t, J=6.93 Hz), 1.31 (32H, m), 1.61 (8H, m), 2.04 (4H, q, J=7.25 Hz), 2.23 (2H, t, J=7.98 Hz), 2.29 (2H, t, J=7.76 Hz), 2.44 (6H, m), 2.77 (2H, t, J=6.34 Hz), 3.26 (2H, m), 3.70 (4H, t, J=4.65 Hz), 4.05 (2H, t, J=6.75 Hz), 4.15 (2H, t, J=6.84 Hz), 4.35 (1H, dd, J=11.43, 2.42 Hz), 4.44 (1H, dd, J=11.43, 4.39 Hz), 4.81 (1H, m), 5.19 (1H, m), 5.34 (4H, m), 6.36 (1H, d, J=7.86 Hz). MS [ESI]: m/z: [M+H] calc. 792.6 obs. 792.7.

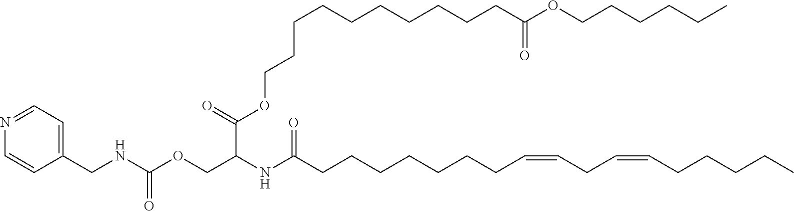 [SMILES: CCCCC/C=C\C/C=C\CCCCCCCC(=O)NC(COC(=O)NCc1ccncc1)C(=O)OCCCCCCCCCCC(=O)OCCCCCC]

Hexyl 11-((N-((9Z,12Z)-octadeca-9,12-dienoyl)-O-((pyridin-4-ylmethyl)carbamoyl)seryl)oxy)undecanoate was synthesized according to representative synthetic scheme 1 and the corresponding methods. ¹H NMR (400 MHz, CDCl₃): δ 0.89 (6H, t, J=7.11 Hz), 1.30 (32H, m), 1.61 (8H, m), 2.04 (4H, q, J=7.17 Hz), 2.22 (2H, t, J=7.69 Hz), 2.28 (2H, t, J=7.59 Hz), 2.76 (2H, t, J=6.39 Hz), 4.05 (2H, t, J=6.75 Hz), 4.15 (2H, t, J=6.31 Hz), 4.37 (2H, d, J=6.41 Hz), 4.42 (1H, dd, J=11.56, 3.26 Hz), 4.49 (1H, dd, J=11.56, 4.16 Hz), 4.83 (1H, m), 5.20 (111, m), 5.34 (4H, m), 6.30 (1H, d, J=7.69 Hz), 7.19 (2H, d, J=6.03 Hz), 8.57 (2H, d, J=6.03 Hz). MS [ESI]: m/z: [M+H] calc. 770.6 obs. 770.7.

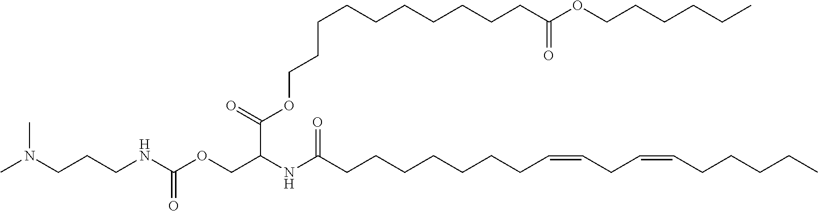 [SMILES: CCCCC/C=C\C/C=C\CCCCCCCC(=O)NC(COC(=O)NCCCN(C)C)C(=O)OCCCCCCCCCCC(=O)OCCCCCC]

Hexyl 11-((O-((3-(dimethylamino)propyl)carbamoyl)-N-((9Z,12Z)-octadeca-9,12-dienoyl)seryl)oxy)undecanoate was synthesized according to representative synthetic scheme 1 and the corresponding methods. ¹H NMR (400 MHz, CDCl₃): δ 0.89 (6H, t, J=7.07 Hz), 1.30 (32H, m), 1.63 (101H, m), 2.04 (4H, q, J=7.13 Hz), 2.20 (12H, m), 2.77 (2H, t, J=6.56 Hz), 3.23 (2H, m), 4.05 (2H, t, J=6.66 Hz), 4.14 (2H, t, J=6.75 Hz), 4.33 (1H, dd, J=11.60, 3.05 Hz), 4.43 (1H, dd, J=11.60, 4.63 Hz), 4.78 (1H, m), 5.34 (4H, m), 5.73 (1H, m), 6.41 (1H, d, J=7.78 Hz). MS [ESI]: m/z: [M+H] calc. 764.6 obs. 764.7.

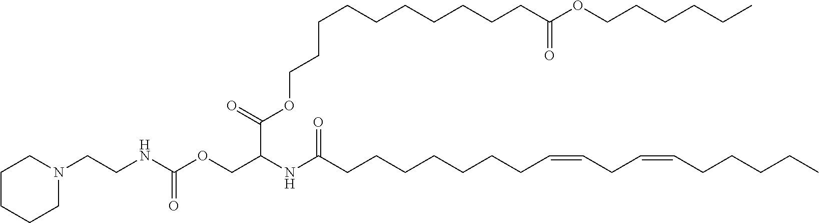 [SMILES: CCCCC/C=C\C/C=C\CCCCCCCC(=O)NC(COC(=O)NCCN1CCCCC1)C(=O)OCCCCCCCCCCC(=O)OCCCCCC]

Hexyl 11-((N-((9Z,12Z)-octadeca-9,12-dienoyl)-O-((2-(piperidin-1-yl)ethyl)carbamoyl)seryl)oxy)undecanoate was synthesized according to representative synthetic scheme 1 and the corresponding methods. ¹H NMR (400 MHz, CDCl₃): δ 0.89 (6H, t, J=7.04 Hz), 1.31 (34H, m), 1.61 (12H, m), 2.04 (4H, q, J=7.03 Hz), 2.28 (10H, m), 2.77 (2H, t, J=6.62 Hz), 3.24 (2H, m), 4.05 (2H, t, J=6.75 Hz), 4.15 (2H, t, J=6.75 Hz), 4.34 (1H, dd, J=11.59, 2.82 Hz), 4.44 (1H, dd, J=11.59, 4.33 Hz), 4.80 (1H, m), 5.34 (5H, m), 6.41 (1H, d, J=8.03 Hz). MS [ESI]: m/z: [M+H] calc. 790.6 obs. 790.8.

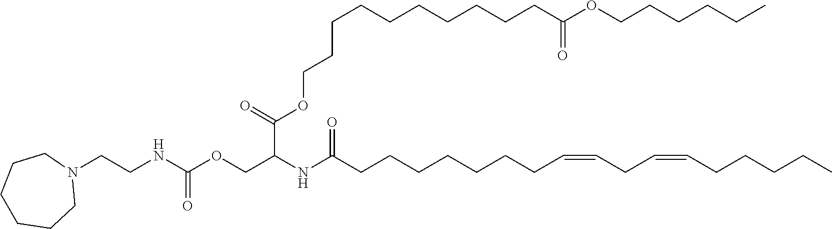 [SMILES: CCCCC/C=C\C/C=C\CCCCCCCC(=O)NC(COC(=O)NCCN1CCCCCC1)C(=O)OCCCCCCCCCCC(=O)OCCCCCC]

Hexyl 11-((O-((2-(azepan-1-yl)ethyl)carbamoyl)-N-((9Z,12Z)-octadeca-9,12-dienoyl)seryl)oxy)undecanoate was synthesized according to representative synthetic scheme 1 and the corresponding methods. ¹H NMR (400 MHz, CDCl₃): δ 0.89 (6H, t, J=6.89 Hz), 1.31 (32H, m), 1.61 (16H, m), 2.04 (4H, q, J=7.26 Hz), 2.28 (4H, m), 2.61 (6H, m), 2.77 (2H, t, J=6.57 Hz), 3.19 (2H, m), 4.05 (2H, t, J=6.75 Hz), 4.15 (2H, t, J=7.59 Hz), 4.34 (1H, dd, J=11.36, 2.55 Hz), 4.45 (1H, dd, J=11.36, 4.36 Hz), 4.80 (1H, m), 5.34 (4H, m), 6.42 (2H, m). MS [ESI]: m/z: [M+H] calc. 804.6 obs. 804.8.

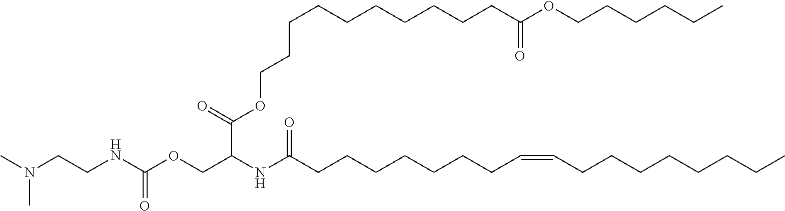 [SMILES: CCCCCCCC/C=C\CCCCCCCC(=O)NC(COC(=O)NCCN(C)C)C(=O)OCCCCCCCCCCC(=O)OCCCCCC]

Hexyl 11-((O-((2-(dimethylamino)ethyl)carbamoyl)-N-oleoylseryl)oxy)undecanoate was synthesized according to representative synthetic scheme 1 and the corresponding methods. ¹H NMR (400 MHz, CDCl₃): δ 0.88 (6H, m), 1.30 (38H, m), 1.63 (8H, m), 2.00 (4H, m), 2.22 (8H, m), 2.29 (2H, t, J=7.69 Hz), 2.39 (2H, t, J=5.88 Hz), 3.23 (2H, m), 4.06 (2H, t, J=6.75 Hz), 4.15 (2H, t, J=6.75 Hz), 4.33 (1H, dd, J=11.30, 3.00 Hz), 4.44 (1H, dd, J=11.30, 4.30 Hz), 4.79 (1H, m), 5.25 (1H, m), 5.33 (2H, m), 6.37 (1H, d, J=7.90 Hz). MS [ESI]: m/z: [M+H] calc. 752.6 obs. 752.3.

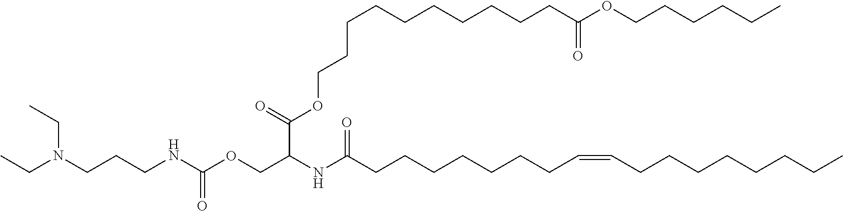 [SMILES: CCCCCCCC/C=C\CCCCCCCC(=O)NC(COC(=O)NCCCN(CC)CC)C(=O)OCCCCCCCCCCC(=O)OCCCCCC]

Hexyl 11-((O-((3-(diethylamino)propyl)carbamoyl)-N-oleoylseryl)oxy)undecanoate was synthesized according to representative synthetic scheme 1 and the corresponding methods. ¹H NMR (400 MHz, CDCl₃): δ 0.88 (6H, m), 1.02 (6H, t, J=7.13 Hz), 1.30 (38H, m), 1.62 (10H, m), 2.00 (4H, m), 2.22 (2H, t, J=7.74 Hz), 2.29 (2H, t, J=7.74 Hz), 2.49 (6H, m), 3.24 (2H, m), 4.06 (2H, t, J=6.75 Hz), 4.13 (2H, t, J=6.83 Hz), 4.33 (1H, dd, J=11.50, 3.09 Hz), 4.42 (1H, dd, J=11.50, 4.67 Hz), 4.77 (1H, m), 5.34 (2H, m), 6.40 (2H, m). MS [ESI]: m/z: [M+H] calc. 794.7 obs. 794.4.

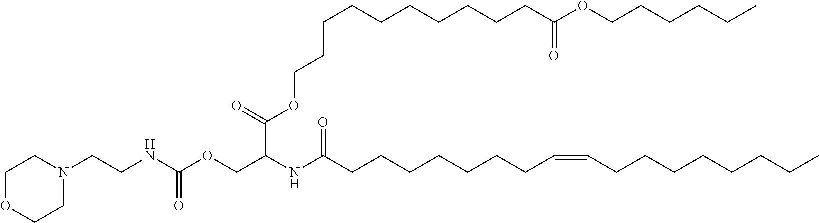 [SMILES: CCCCCCCC/C=C\CCCCCCCC(=O)NC(COC(=O)NCCN1CCOCC1)C(=O)OCCCCCCCCCCC(=O)OCCCCCC]

Hexyl 11-((O-((2-morpholinoethyl)carbamoyl)-N-oleoylseryl)oxy)undecanoate was synthesized according to representative synthetic scheme 1 and the corresponding methods. ¹H NMR (400 MHz, CDCl₃): δ 0.88 (6H, m), 1.30 (38H, m), 1.61 (8H, m), 2.00 (4H, m), 2.23 (2H, t, J=7.96 Hz), 2.29 (2H, t, J=7.66 Hz), 2.44 (6H, m), 3.26 (2H, m), 3.70 (4H, t, J=4.34 Hz), 4.05 (2H, t, J=6.75 Hz), 4.15 (2H, t, J=6.76 Hz), 4.35 (1H, dd, J=11.70, 3.09 Hz), 4.44 (1H, dd, J=11.70, 4.49 Hz), 4.81 (1H, m), 5.20 (1H, m), 5.34 (2H, m), 6.36 (1H, d, J=7.60 Hz). MS [ESI]: m/z: [M+H] calc. 794.6 obs. 794.4.

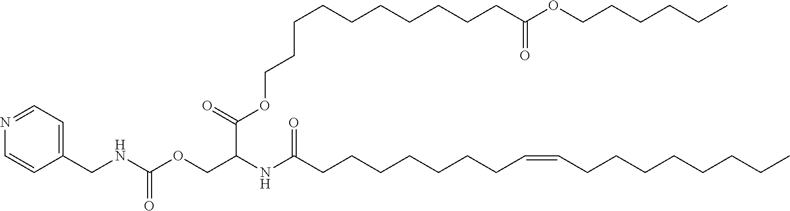 [SMILES: CCCCCCCC/C=C\CCCCCCCC(=O)NC(COC(=O)NCc1ccncc1)C(=O)OCCCCCCCCCCC(=O)OCCCCCC]

Hexyl 11-((N-oleoyl-O-((pyridin-4-ylmethyl)carbamoyl)seryl)oxy)undecanoate was synthesized according to representative synthetic scheme 1 and the corresponding methods. ¹H NMR (400 MHz, CDCl₃): δ 0.88 (6H, m), 1.27 (38H, m), 1.64 (8H, m), 2.01 (4H, m), 2.28 (4H, m), 4.05 (2H, t, J=6.77 Hz), 4.15 (2H, t, J=6.25 Hz), 4.37 (2H, d, J=6.25 Hz), 4.44 (2H, m), 4.83 (1H, m), 5.18 (1H, m), 5.34 (2H, m), 6.29 (1H, d, J=7.60 Hz), 7.19 (2H, d, J=5.02 Hz), 8.56 (2H, m). MS [ESI]: m/z: [M+H] calc. 772.6 obs. 772.4.

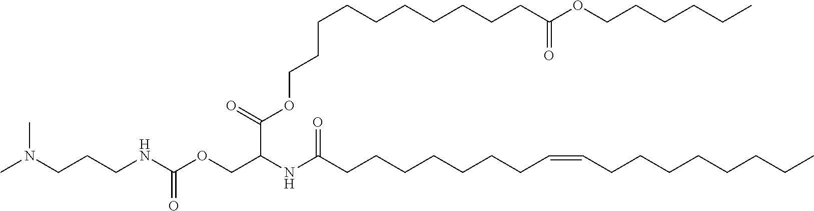 [SMILES: CCCCCCCC/C=C\CCCCCCCC(=O)NC(COC(=O)NCCCN(C)C)C(=O)OCCCCCCCCCCC(=O)OCCCCCC]

Hexyl 11-((O-((3-(dimethylamino)propyl)carbamoyl)-N-oleoylseryl)oxy)undecanoate was synthesized according to representative synthetic scheme 1 and the corresponding methods. ¹H NMR (400 MHz, CDCl₃): δ 0.88 (6H, m), 1.30 (38H, m), 1.62 (10H, m), 2.00 (4H, m), 2.21 (8H, m), 2.29 (2H, t, J=7.63 Hz), 2.34 (2H, t, J=6.66 Hz), 3.24 (2H, m), 4.06 (2H, t, J=6.75 Hz), 4.14 (2H, t, J=6.82 Hz), 4.34 (1H, dd, J=11.60, 3.21 Hz), 4.43 (1H, dd, J=11.60, 4.42 Hz), 4.78 (1H, m), 5.34 (2H, m), 5.72 (1H, m), 6.41 (1H, d, J=7.67 Hz). MS [ESI]: m/z: [M+H] calc. 766.6 obs. 766.9.

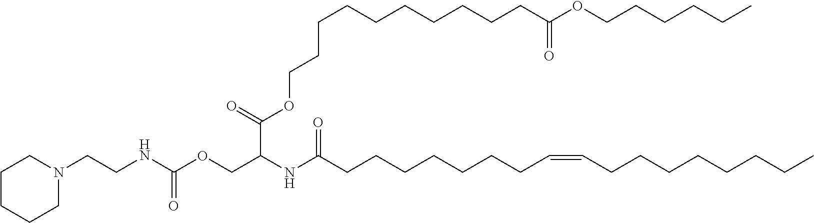 [SMILES: CCCCCCCC/C=C\CCCCCCCC(=O)NC(COC(=O)NCCN1CCCCC1)C(=O)OCCCCCCCCCCC(=O)OCCCCCC]

Hexyl 11-((N-oleoyl-O-((2-(piperidin-1-yl)ethyl)carbamoyl)seryl)oxy)undecanoate was synthesized according to representative synthetic scheme 1 and the corresponding methods. ¹H NMR (400 MHz, CDCl₃): δ 0.89 (6H, m), 1.31 (38H, m), 1.44 (2H, m), 1.58 (12H, m), 2.01 (4H, m), 2.24 (2H, t, J=7.83 Hz), 2.29 (2H, t, J=7.56 Hz), 2.41 (6H, m), 3.24 (2H, m), 4.06 (2H, t, J=6.75 Hz), 4.15 (2H, t, J=6.75 Hz), 4.34 (1H, dd, J=11.58, 3.18 Hz), 4.45 (1H, dd, J=11.58, 4.17 Hz), 4.80 (1H, m), 5.34 (3H, m), 6.41 (1H, d, J=8.16 Hz). MS [ESI]: m/z: [M+H] calc. 792.6 obs. 792.4.

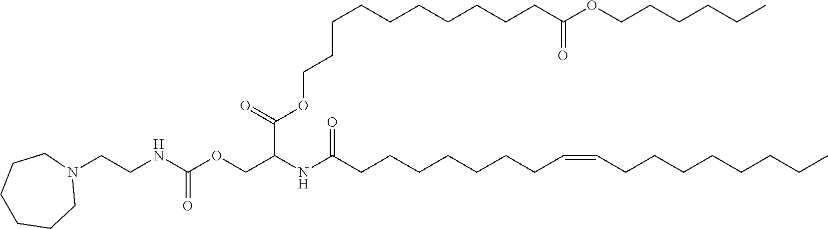 [SMILES: CCCCCCCC/C=C\CCCCCCCC(=O)NC(COC(=O)NCCN1CCCCCC1)C(=O)OCCCCCCCCCCC(=O)OCCCCCC]

Hexyl 11-((O-((2-(azepan-1-yl)ethyl)carbamoyl)-N-oleoylseryl)oxy)undecanoate was synthesized according to representative synthetic scheme 1 and the corresponding methods. ¹H NMR (400 MHz, CDCl₃): δ 0.89 (6H, m), 1.30 (38H, m), 1.61 (16H, m), 2.01 (4H, m), 2.29 (4H, m), 2.62 (6H, m), 3.20 (2H, m), 4.06 (2H, t, J=6.75 Hz), 4.15 (2H, t, J=6.88 Hz), 4.34 (1H, dd, J=11.36, 3.10 Hz), 4.45 (1H, dd, J=11.36, 4.53 Hz), 4.80 (1H, m), 5.34 (3H, m), 6.41 (1H, d, J=7.58 Hz). MS [ESI]: m/z: [M+H] calc. 806.7 obs. 806.7.

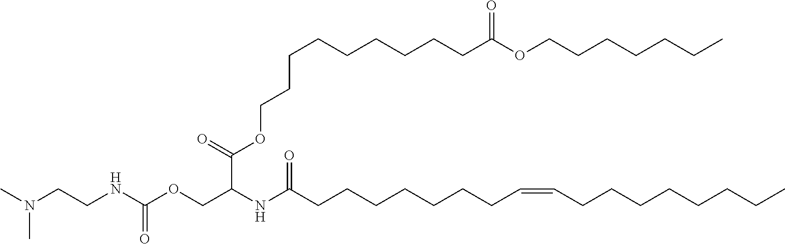 [SMILES: CCCCCCCC/C=C\CCCCCCCC(=O)NC(COC(=O)NCCN(C)C)C(=O)OCCCCCCCCCC(=O)OCCCCCCC]

Heptyl 10-((O-((2-(dimethylamino)ethyl)carbamoyl)-N-oleoylseryl)oxy)decanoate was synthesized according to representative synthetic scheme 1 and the corresponding methods. ¹H NMR (400 MHz, CDCl₃): δ 0.88 (6H, m), 1.30 (38H, m), 1.62 (8H, m), 2.00 (4H, q, J=6.18 Hz), 2.22 (8H, m), 2.28 (2H, t, J=7.60 Hz), 2.39 (2H, t, J=6.03 Hz), 3.23 (2H, m), 4.05 (2H, t, J=6.66 Hz), 4.14 (2H, t, J=6.75 Hz), 4.33 (1H, dd, J=11.58, 3.29 Hz), 4.44 (1H, dd, J=11.58, 4.11 Hz), 4.79 (1H, m), 5.34 (3H, m), 6.39 (1H, d, J=7.86 Hz). MS [ESI]: m/z: [M+H] calc. 752.6 obs. 752.3.

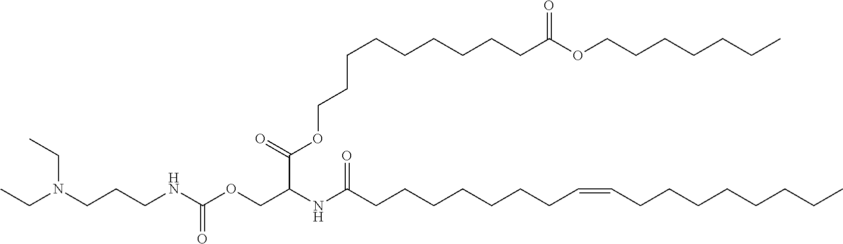 [SMILES: CCCCCCCC/C=C\CCCCCCCC(=O)NC(COC(=O)NCCCN(CC)CC)C(=O)OCCCCCCCCCC(=O)OCCCCCCC]

Heptyl 10-((O-((3-(diethylamino)propyl)carbamoyl)-N-oleoylseryl)oxy)decanoate was synthesized according to representative synthetic scheme 1 and the corresponding methods. ¹H NMR (500 MHz, CDCl₃): δ 0.88 (6H, m), 1.01 (6H, t, J=7.05 Hz), 1.29 (38H, m), 1.61 (10H, m), 2.00 (4H, q, J=6.59 Hz), 2.22 (2H, t, J=7.89 Hz), 2.28 (2H, t, J=7.58 Hz), 2.48 (6H, m), 3.24 (2H, q, J=5.91 Hz), 4.05 (2H, t, J=6.73 Hz), 4.13 (2H, t, J=7.02 Hz), 4.33 (1H, dd, J=11.41, 3.41 Hz), 4.41 (1H, dd, J=11.41, 4.37 Hz), 4.76 (1H, m), 5.34 (2H, m), 6.41 (2H, m). MS [ESI]: m/z: [M+H] calc. 794.7 obs. 794.8.

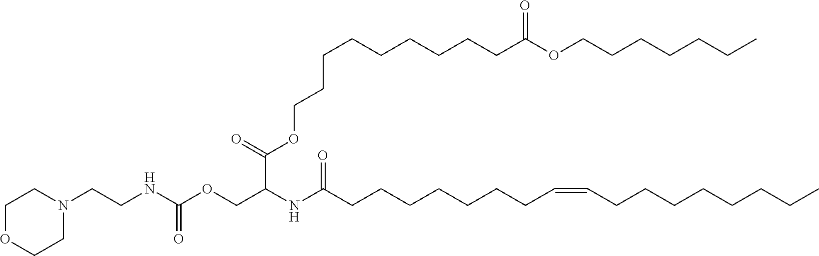 [SMILES: CCCCCCCC/C=C\CCCCCCCC(=O)NC(COC(=O)NCCN1CCOCC1)C(=O)OCCCCCCCCCC(=O)OCCCCCCC]

Heptyl 10-((O-((2-morpholinoethyl)carbamoyl)-N-oleoylseryl)oxy)decanoate was synthesized according to representative synthetic scheme 1 and the corresponding methods. ¹H NMR (500 MHz, CDCl₃): δ 0.88 (6H, m), 1.29 (38H, m), 1.62 (8H, m), 2.01 (4H, q, J=6.64 Hz), 2.23 (2H, t, J=7.75 Hz), 2.28 (2H, t, J=7.65 Hz), 2.45 (6H, m), 3.23-3.30 (2H, m), 3.70 (4H, t, J=4.80 Hz), 4.05 (2H, t, J=6.73 Hz), 4.15 (2H, td, J=6.79, 1.87 Hz), 4.35 (1H, dd, J=11.29, 3.33 Hz), 4.44 (1H, dd, J=11.29, 4.28 Hz), 4.80 (1H, m), 5.21 (1H, m), 5.34 (2H, m), 6.37 (1H, d, J=7.59 Hz). MS [ESI]: m/z: [M+H] calc. 794.6 obs. 794.3.

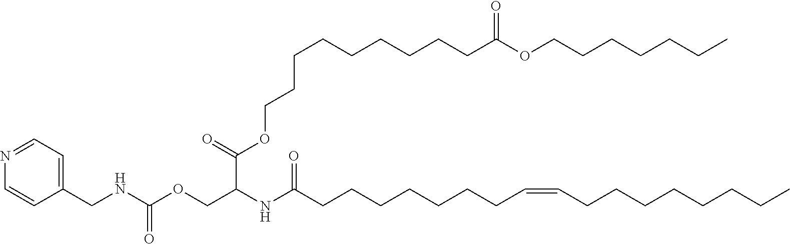 [SMILES: CCCCCCCC/C=C\CCCCCCCC(=O)NC(COC(=O)NCc1ccncc1)C(=O)OCCCCCCCCCC(=O)OCCCCCCC]

Heptyl 10-((N-oleoyl-O-((pyridin-4-ylmethyl)carbamoyl)seryl)oxy)decanoate was synthesized according to representative synthetic scheme 1 and the corresponding methods. ¹H NMR (500 MHz, CDCl₃): δ 0.88 (6H, m), 1.30 (38H, m), 1.62 (8H, m), 2.00 (4H, q, J=6.49 Hz), 2.22 (2H, t, J=7.48 Hz), 2.28 (2H, t, J=7.55 Hz), 4.05 (2H, t, J=6.91 Hz), 4.14 (2H, m), 4.37 (2H, d, J=6.32 Hz), 4.42 (1H, dd, J=11.65, 3.28 Hz), 4.49 (1H, dd, J=11.65, 4.36 Hz), 4.83 (1H, m), 5.22 (1H, t, J=6.43 Hz), 5.33 (2H, m), 6.31 (1H, d, J=7.65 Hz), 7.19 (2H, d, J=5.99 Hz), 8.57 (2H, d, J=5.99 Hz). MS [ESI]: m/z: [M+H] calc. 772.6 obs. 772.4.

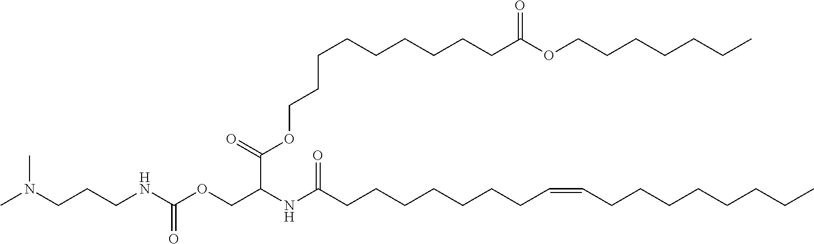 [SMILES: CCCCCCCC/C=C\CCCCCCCC(=O)NC(COC(=O)NCCCN(C)C)C(=O)OCCCCCCCCCC(=O)OCCCCCCC]

Heptyl 10-((O-((3-(dimethylamino)propyl)carbamoyl)-N-oleoylseryl)oxy)decanoate was synthesized according to representative synthetic scheme 1 and the corresponding methods. ¹H NMR (500 MHz, CDCl₃): δ 0.88 (6H, m), 1.29 (38H, m), 1.62 (10H, m), 2.00 (4H, q, J=6.22 Hz), 2.21 (8H, m), 2.28 (2H, t, J=7.67 Hz), 2.33 (2H, t, J=6.68 Hz), 3.23 (2H, m), 4.05 (2H, t, J=6.73 Hz), 4.13 (2H, t, J=6.66 Hz), 4.33 (1H, dd, J=11.74, 3.42 Hz), 4.43 (1H, dd, J=11.74, 4.35 Hz), 4.77 (1H, m), 5.34 (2H, m), 5.74 (1H, m), 6.42 (1H, d, J=7.80 Hz). MS [ESI]: m/z: [M+H] calc. 766.6 obs. 766.4.

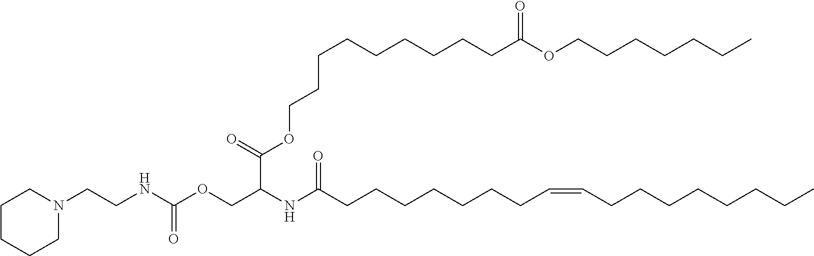 [SMILES: CCCCCCCC/C=C\CCCCCCCC(=O)NC(COC(=O)NCCN1CCCCC1)C(=O)OCCCCCCCCCC(=O)OCCCCCCC]

Heptyl 10-((N-oleoyl-O-((2-(piperidin-1-yl)ethyl)carbamoyl)seryl)oxy)decanoate was synthesized according to representative synthetic scheme 1 and the corresponding methods. ¹H NMR (500 MHz, CDCl₃): δ 0.87 (6H, m), 1.29 (38H, m), 1.43 (2H, m), 1.62 (12H, m), 2.00 (4H, q, J=6.44 Hz), 2.24 (2H, t, J=7.65 Hz), 2.28 (2H, t, J=7.70 Hz), 2.38 (6H, m), 3.24 (2H, m), 4.05 (2H, t, J=6.73 Hz), 4.15 (2H, t, J=6.84 Hz), 4.34 (1H, dd, J=11.34, 2.94 Hz), 4.44 (1H, dd, J=11.34, 4.26 Hz), 4.79 (1H, m), 5.34 (3H, m), 6.42 (1H, d, J=8.08 Hz). MS [ESI]: m/z: [M+H] calc. 792.6 obs. 792.7.

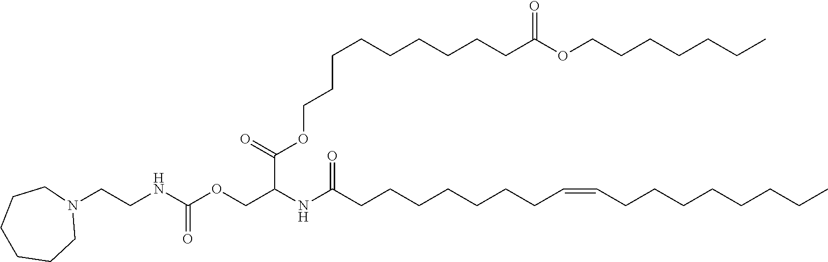 [SMILES: CCCCCCCC/C=C\CCCCCCCC(=O)NC(COC(=O)NCCN1CCCCCC1)C(=O)OCCCCCCCCCC(=O)OCCCCCCC]

Heptyl 10-((O-((2-(azepan-1-yl)ethyl)carbamoyl)-N-oleoylseryl)oxy)decanoate was synthesized according to representative synthetic scheme 1 and the corresponding methods. ¹H NMR (500 MHz, CDCl₃): δ 0.88 (6H, m), 1.30 (38H, m), 1.54 (16H, m), 2.01 (4H, m), 2.29 (2H, m), 2.62 (8H, m), 3.11-3.29 (2H, m), 4.03-4.09 (2H, m), 4.10-4.18 (2H, m), 4.28-4.50 (2H, m), 4.75-4.87 (1H, m), 5.29-5.41 (1H, m), 6.34-6.47 (1H, m). MS [ESI]: m/z: [M+H] calc. 806.7 obs. 806.5.

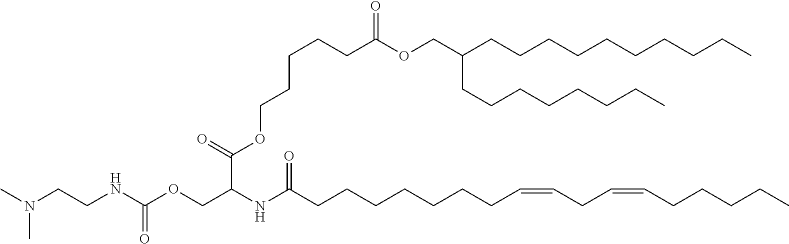 [SMILES: CCCCC/C=C\C/C=C\CCCCCCCC(=O)NC(COC(=O)NCCN(C)C)C(=O)OCCCCCC(=O)OCC(CCCCCCCC)CCCCCCCCCC]

2-Octyldodecyl 6-((O-((2-(dimethylamino)ethyl)carbamoyl)-N-((9Z,12Z)-octadeca-9,12-dienoyl)seryl)oxy)hexanoate was synthesized according to representative synthetic scheme 1 and the corresponding methods. ¹H NMR (400 MHz, CDCl₃): δ 0.88 (9H, m), 1.26 (48H, m), 1.65 (7H, m), 2.05 (4H, q, J=7.13 Hz), 2.22 (8H, m), 2.31 (2H, t, J=7.43 Hz), 2.39 (2H, t, J=5.97 Hz), 2.77 (2H, t, J=6.61 Hz), 3.23 (2H, m), 3.96 (2H, d, J=5.81 Hz), 4.16 (2H, td, J=6.83, 2.13 Hz), 4.34 (1H, dd, J=11.36, 3.31 Hz), 4.43 (1H, dd, J=11.54, 3.98 Hz), 4.79 (1H, m), 5.34 (5H, m), 6.42 (1H, d, J=7.94 Hz). MS [ESI]: m/z: [M+H] calc. 876.7 obs. 876.8.

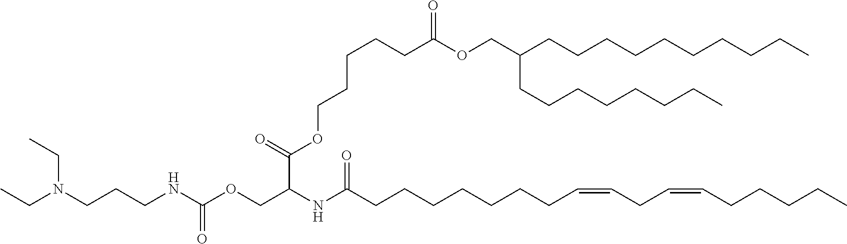 [SMILES: CCCCC/C=C\C/C=C\CCCCCCCC(=O)NC(COC(=O)NCCCN(CC)CC)C(=O)OCCCCCC(=O)OCC(CCCCCCCC)CCCCCCCCCC]

2-Octyldodecyl 6-((O-((3-(diethylamino)propyl)carbamoyl)-N-((9Z,12Z)-octadeca-9,12-dienoyl)seryl)oxy)hexanoate was synthesized according to representative synthetic scheme 1 and the corresponding methods. ¹H NMR (400 MHz, CDCl₃): δ 0.88 (9H, m), 1.01 (6H, m), 1.26 (48H, m), 1.63 (9H, m), 2.05 (4H, q, J=7.22 Hz), 2.23 (2H, t, J=8.12 Hz), 2.31 (2H, t, J=7.62 Hz), 2.48 (6H, m), 2.77 (2H, t, J=6.46 Hz), 3.23 (2H, m), 3.96 (2H, d, J=5.81 Hz), 4.14 (2H, q, J=6.66 Hz), 4.34 (1H, dd, J=11.44, 3.43 Hz), 4.41 (1H, dd, J=11.58, 4.46 Hz), 4.76 (1H, m), 5.34 (4H, m), 6.44 (2H, m). MS [ESI]: m/z: [M+H] calc. 918.8 obs. 918.9.

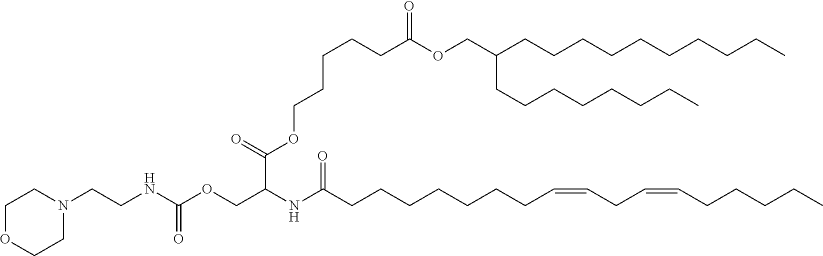 [SMILES: CCCCC/C=C\C/C=C\CCCCCCCC(=O)NC(COC(=O)NCCN1CCOCC1)C(=O)OCCCCCC(=O)OCC(CCCCCCCC)CCCCCCCCCC]

2-Octyldodecyl 6-((O-((2-morpholinoethyl)carbamoyl)-N-((9Z,12Z)-octadeca-9,12-dienoyl)seryl)oxy)hexanoate was synthesized according to representative synthetic scheme 1 and the corresponding methods. ¹H NMR (400 MHz, CDCl₃): δ 0.88 (9H, m), 1.26 (48H, m), 1.58 (7H, m), 2.05 (4H, q, J=7.43 Hz), 2.24 (2H, t, J=8.04 Hz), 2.31 (2H, t, J=7.47 Hz), 2.45 (6H, m), 2.77 (2H, t, J=6.56 Hz), 3.27 (2H, m), 3.70 (4H, m), 3.96 (2H, d, J=5.64 Hz), 4.17 (2H, t, J=6.86 Hz), 4.36 (1H, dd, J=11.86, 3.66 Hz), 4.44 (1H, dd, J=11.86, 4.49 Hz), 4.81 (1H, m), 5.34 (5H, m), 6.41 (1H, d, J=7.70 Hz). MS [ESI]: m/z: [M+H] calc. 918.8 obs. 918.9.

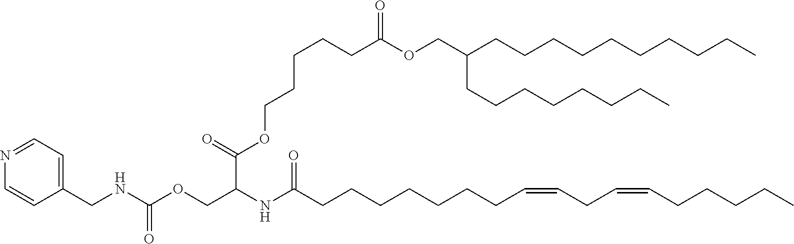 [SMILES: CCCCC/C=C\C/C=C\CCCCCCCC(=O)NC(COC(=O)NCc1ccncc1)C(=O)OCCCCCC(=O)OCC(CCCCCCCC)CCCCCCCCCC]

2-Octyldodecyl 6-((N-((9Z,12Z)-octadeca-9,12-dienoyl)-O-((pyridin-4-ylmethyl)carbamoyl)seryl)oxy)hexanoate was synthesized according to representative synthetic scheme 1 and the corresponding methods. 1H NMR (400 MHz, CDCl₃): δ 0.88 (9H, m), 1.26 (48H, m), 1.58 (7H, m), 2.04 (4H, q, J=7.34 Hz), 2.22 (2H, t, J=7.48 Hz), 2.30 (2H, t, J=7.30 Hz), 2.77 (2H, t, J=6.48 Hz), 3.93 (2H, d, J=5.64 Hz), 4.18 (2H, m), 4.37 (2H, d, J=6.21 Hz), 4.46 (2H, d, J=3.59 Hz), 4.81 (1H, m), 5.34 (4H, m), 5.55 (1H, t, J=6.43 Hz), 6.39 (1H, d, J=7.77 Hz), 7.19 (2H, d, J=6.01 Hz), 8.56 (2H, d, J=6.01 Hz). MS [ESI]: m/z: [M+H] calc. 896.7 obs. 896.9.

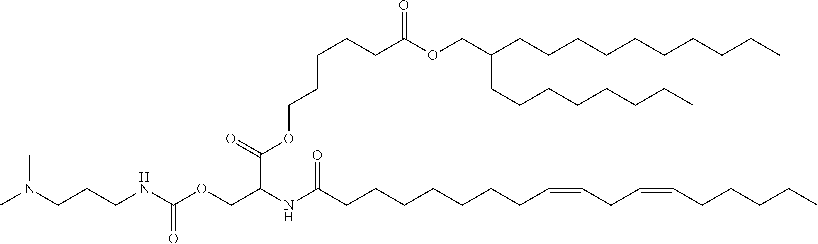 [SMILES: CCCCC/C=C\C/C=C\CCCCCCCC(=O)NC(COC(=O)NCCCN(C)C)C(=O)OCCCCCC(=O)OCC(CCCCCCCC)CCCCCCCCCC]

2-Octyldodecyl 6-((O-((3-(dimethylamino)propyl)carbamoyl)-N-((9Z,12Z)-octadeca-9,12-dienoyl)seryl)oxy)hexanoate was synthesized according to representative synthetic scheme 1 and the corresponding methods. ¹H NMR (400 MHz, CDCl₃): δ 0.88 (9H, m), 1.26 (48H, m), 1.64 (9H, m), 2.04 (4H, q, J=7.07 Hz), 2.21 (8H, m), 2.31 (4H, m), 2.77 (2H, t, J=6.60 Hz), 3.23 (2H, m), 3.96 (2H, d, J=5.72 Hz), 4.13 (2H, m), 4.34 (1H, dd, J=11.79, 3.00 Hz), 4.42 (1H, dd, J=11.79, 4.39 Hz), 4.77 (1H, m), 5.34 (4H, m), 5.79 (1H, t, J=5.30 Hz), 6.46 (1H, d, J=7.60 Hz). MS [ESI]: m/z: [M+H] calc. 890.8 obs. 890.9.

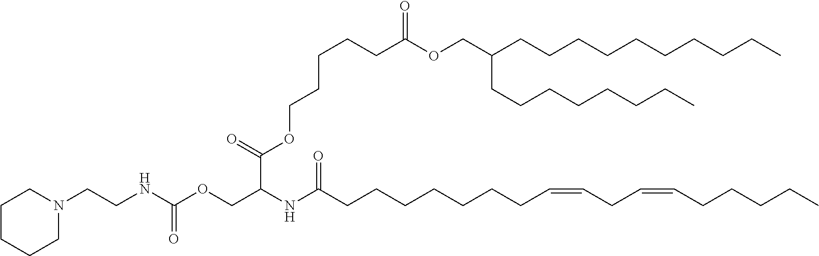 [SMILES: CCCCC/C=C\C/C=C\CCCCCCCC(=O)NC(COC(=O)NCCN1CCCCC1)C(=O)OCCCCCC(=O)OCC(CCCCCCCC)CCCCCCCCCC]

2-Octyldodecyl 6-((N-((9Z,12Z)-octadeca-9,12-dienoyl)-O-((2-(piperidin-1-yl)ethyl)carbamoyl)seryl)oxy)hexanoate was synthesized according to representative synthetic scheme 1 and the corresponding methods. ¹H NMR (400 MHz, CDCl₃): δ 0.88 (9H, m), 1.26 (50H, m), 1.67 (11H, m), 2.04 (4H, q, J=7.22 Hz), 2.24 (2H, t, J=7.95 Hz), 2.31 (8H, m), 2.77 (2H, t, J=6.41 Hz), 3.24 (2H, m), 3.96 (2H, d, J=5.64 Hz), 4.16 (2H, t, J=6.57 Hz), 4.34 (1H, dd, J=11.66, 2.97 Hz), 4.44 (1H, dd, J=11.66, 4.23 Hz), 4.79 (1H, m), 5.34 (5H, m), 6.45 (1H, d, J=7.86 Hz). MS [ESI]: m/z: [M+H] calc. 916.8 obs. 917.0.

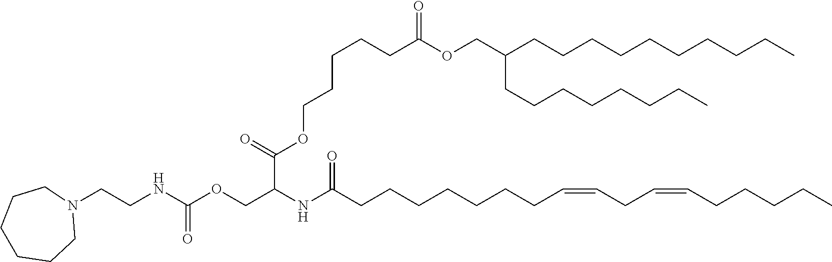 [SMILES: CCCCC/C=C\C/C=C\CCCCCCCC(=O)NC(COC(=O)NCCN1CCCCCC1)C(=O)OCCCCCC(=O)OCC(CCCCCCCC)CCCCCCCCCC]

2-Octyldodecyl 6-((O-((2-(azepan-1-yl)ethyl)carbamoyl)-N-((9Z,12Z)-octadeca-9,12-dienoyl)seryl)oxy)hexanoate was synthesized according to representative synthetic scheme 1 and the corresponding methods. ¹H NMR (400 MHz, CDCl₃): δ 0.88 (9H, m), 1.26 (48H, m), 1.63 (15H, m), 2.04 (4H, q, J=7.26 Hz), 2.24 (2H, t, J=7.84 Hz), 2.31 (2H, t, J=7.60 Hz), 2.62 (6H, m), 2.77 (2H, t, J=6.47 Hz), 3.20 (2H, m), 3.96 (2H, d, J=5.81 Hz), 4.16 (2H, t, J=6.77 Hz), 4.34 (1H, dd, J=11.47, 2.95 Hz), 4.44 (1H, dd, J=11.47, 4.29 Hz), 4.79 (1H, m), 5.34 (5H, m), 6.45 (1H, d, J=7.69 Hz). MS [ESI]: m/z: [M+H] calc. 930.8 obs. 930.9.

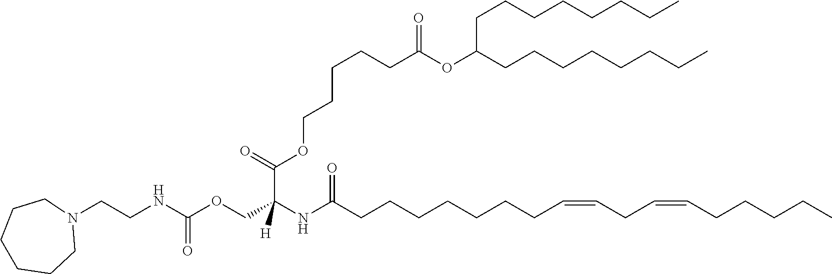 [SMILES: CCCCC/C=C\C/C=C\CCCCCCCC(=O)N[C@H](COC(=O)NCCN1CCCCCC1)C(=O)OCCCCCC(=O)OC(CCCCCCCC)CCCCCCCC]

Heptadecan-9-yl 8-((O-((2-(azepan-1-yl)ethyl)carbamoyl)-N-((9Z,12Z)-octadeca-9,12-dienoyl)-D-seryl)oxy)octanoate was synthesized according to representative synthetic scheme 1 and the corresponding methods. ¹H NMR (400 MHz, CDCl₃): δ 0.88 (9H, m), 1.26 (44H, m), 1.59 (18H, m), 2.05 (4H, q, J=7.30 Hz), 2.26 (4H, m), 2.59 (6H, m), 2.77 (2H, t, J=6.55 Hz), 3.20 (2H, m), 4.15 (2H, t, J=6.83 Hz), 4.34 (1H, dd, J=11.45, 2.83 Hz), 4.45 (1H, dd, J=11.45, 4.35 Hz), 4.86 (2H, m), 5.34 (5H, m), 6.42 (1H, d, J=7.69 Hz). MS [ESI]: m/z: [M+H] calc. 916.8 obs. 917.0.

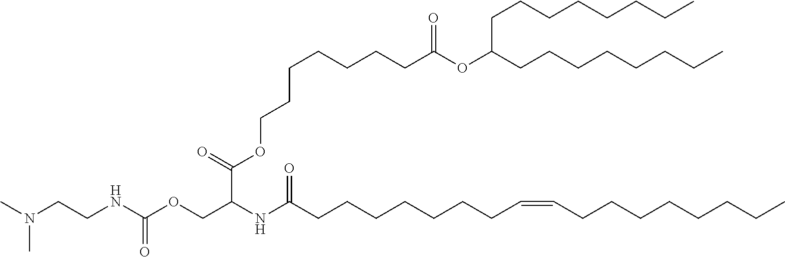 [SMILES: CCCCCCCC/C=C\CCCCCCCC(=O)NC(COC(=O)NCCN(C)C)C(=O)OCCCCCCCC(=O)OC(CCCCCCCC)CCCCCCCC]

Heptadecan-9-yl 8-((O-((2-(dimethylamino)ethyl)carbamoyl)-N-oleoylseryl)oxy)octanoate was synthesized according to representative synthetic scheme 1 and the corresponding methods. ¹H NMR (500 MHz, CDCl₃): δ 0.90 (9H, t, J=7.09 Hz), 1.27 (50H, m), 1.52 (4H, m), 1.65 (6H, m), 2.03 (4H, q, J=6.13 Hz), 2.24 (10H, m), 2.41 (2H, t, J=6.09 Hz), 3.25 (2H, m), 4.16 (2H, t, J=6.83 Hz), 4.36 (1H, dd, J=11.09, 3.22 Hz), 4.46 (1H, dd, J=11.09, 4.11 Hz), 4.81 (1H, m), 4.88 (1H, i, J=6.04 Hz), 5.28 (1H, m), 5.36 (2H, m), 6.41 (1H, d, J=7.77 Hz). MS [ESI]: m/z: [M+H] calc. 864.7 obs. 864.5.

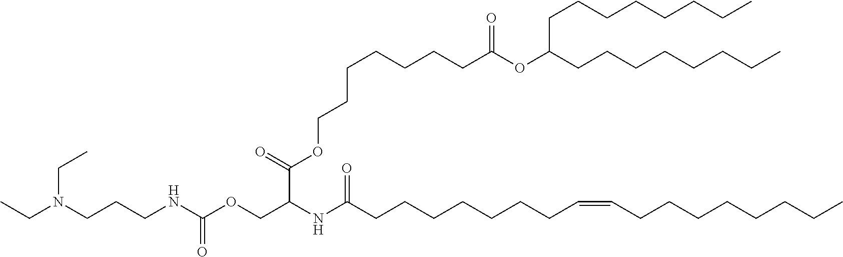 [SMILES: CCCCCCCC/C=C\CCCCCCCC(=O)NC(COC(=O)NCCCN(CC)CC)C(=O)OCCCCCCCC(=O)OC(CCCCCCCC)CCCCCCCC]

Heptadecan-9-yl 8-((O-((3-(diethylamino)propyl)carbamoyl)-N-oleoylseryl)oxy)octanoate was synthesized according to representative synthetic scheme 1 and the corresponding methods. ¹H NMR (500 MHz, CDCl₃): δ 0.89 (9H, t, J=7.24 Hz), 1.03 (6H, t, J=7.16 Hz), 1.27 (50H, m), 1.52 (4H, m), 1.65 (8H, m), 2.02 (4H, q, J=6.21 Hz), 2.24 (2H, t, J=8.02 Hz), 2.29 (2H, t, J=7.65 Hz), 2.49 (6H, m), 3.26 (2H, q, J=5.98 Hz), 4.15 (2H, t, J=6.99 Hz), 4.36 (1H, dd, J=11.45, 3.01 Hz), 4.43 (1H, dd, J=11.45, 4.48 Hz), 4.78 (1H, m), 4.88 (1H, i, J=6.59 Hz), 5.36 (2H, m), 6.43 (2H, m). MS [ESI]: m/z: [M+H] calc 906.8 obs. 906.6.

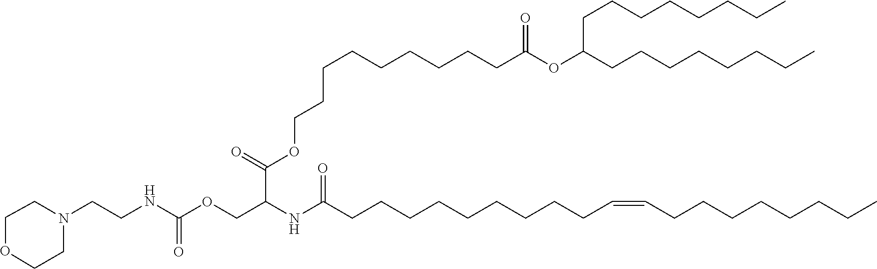 [SMILES: CCCCCCCC/C=C\CCCCCCCCCC(=O)NC(COC(=O)NCCN1CCOCC1)C(=O)OCCCCCCCCCC(=O)OC(CCCCCCCC)CCCCCCCC]

Heptadecan-9-yl 8-((O-((2-morpholinoethyl)carbamoyl)-N-oleoylseryl)oxy)octanoate was synthesized according to representative synthetic scheme 1 and the corresponding methods. ¹H NMR (500 MHz, CDCl₃): δ 0.89 (9H, t, J=7.13 Hz), 1.27 (50H, m), 1.52 (4H, m), 1.65 (6H, m), 2.02 (4H, q, J=6.25 Hz), 2.27 (4H, m), 2.47 (6H, m), 3.29 (2H, m), 3.72 (4H, t, J=4.63 Hz), 4.17 (2H, td, J=7.05, 2.33 Hz), 4.37 (1H, dd, J=11.82, 2.96 Hz), 4.46 (1H, dd, J=11.82, 4.74 Hz), 4.82 (1H, m), 4.87 (1H, i, J=6.15 Hz), 5.24 (1H, m), 5.35 (2H, m), 6.40 (1H, d, J=7.79 Hz). MS [ESI]: m/z: [M+H] calc 906.8 obs. 906.6.

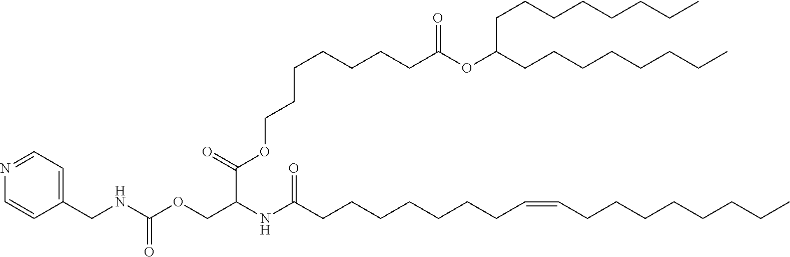 [SMILES: CCCCCCCC/C=C\CCCCCCCC(=O)NC(COC(=O)NCc1ccncc1)C(=O)OCCCCCCCC(=O)OC(CCCCCCCC)CCCCCCCC]

Heptadecan-9-yl 8-((N-oleoyl-O-((pyridin-4-ylmethyl)carbamoyl)seryl)oxy)octanoate was synthesized according to representative synthetic scheme 1 and the corresponding methods. ¹H NMR (500 MHz, CDCl₃): δ 0.89 (9H, t, J=7.05 Hz), 1.27 (50H, m), 1.51 (4H, m), 1.66 (6H, m), 2.02 (4H, q, J=6.69 Hz), 2.24 (2H, t, J=7.90 Hz), 2.29 (2H, t, J=7.52 Hz), 4.17 (2H, t, J=6.50 Hz), 4.39 (2H, d, J=6.31 Hz), 4.45 (1H, dd, J=11.33, 3.06 Hz), 4.50 (1H, dd, J=11.33, 4.41 Hz), 4.85 (2H, m), 5.36 (3H, m), 6.36 (1H, d, J=7.37 Hz), 7.21 (2H, d, J=5.85 Hz), 8.58 (2H, d, J=5.12 Hz). MS [ESI]: m/z: [M+H] calc 884.7 obs. 885.0.

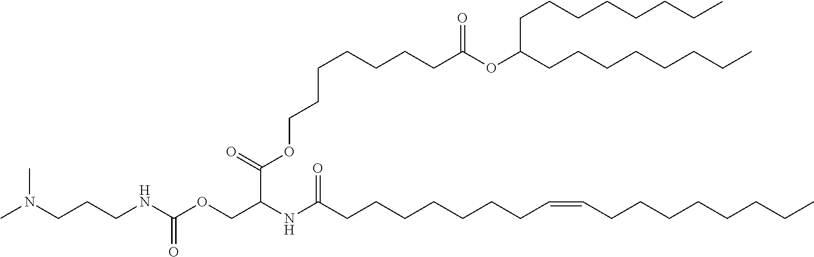 [SMILES: CCCCCCCC/C=C\CCCCCCCC(=O)NC(COC(=O)NCCCN(C)C)C(=O)OCCCCCCCC(=O)OC(CCCCCCCC)CCCCCCCC]

Heptadecan-9-yl 8-((O-((3-(dimethylamino)propyl)carbamoyl)-N-oleoylseryl)oxy)octanoate was synthesized according to representative synthetic scheme 1 and the corresponding methods. ¹H NMR (500 MHz, CDCl₃): δ 0.90 (9H, t, J=7.25 Hz), 1.28 (50H, m), 1.52 (4H, m), 1.66 (8H, m), 2.03 (4H, q, J=6.64 Hz), 2.24 (8H, m), 2.30 (2H, t, J=7.69 Hz), 2.36 (2H, t, J=6.75 Hz), 3.26 (2H, q, J=6.41 Hz), 4.16 (2H, t, J=6.82 Hz), 4.36 (1H, dd, J=11.82, 3.06 Hz), 4.45 (1H, dd, J=11.82, 4.10 Hz), 4.80 (1H, m), 4.88 (1H, i, J=6.46 Hz), 5.36 (2H, m), 5.77 (1H, m), 6.45 (1H, d, J=7.75 Hz). MS [ESI]: m/z: [M+H] calc 878.8 obs. 878.6.

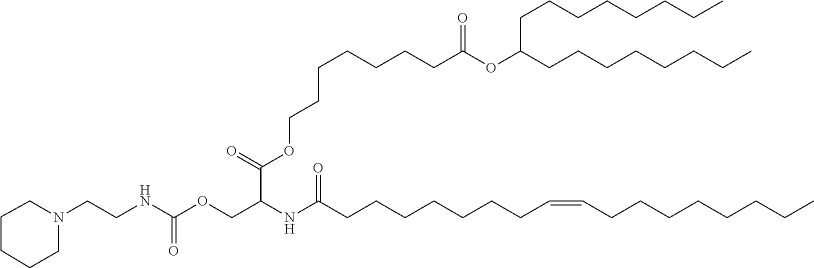 [SMILES: CCCCCCCC/C=C\CCCCCCCC(=O)NC(COC(=O)NCCN1CCCCC1)C(=O)OCCCCCCCC(=O)OC(CCCCCCCC)CCCCCCCC]

Heptadecan-9-yl 8-((N-oleoyl-O-((2-(piperidin-1-yl)ethyl)carbamoyl)seryl)oxy)octanoate was synthesized according to representative synthetic scheme 1 and the corresponding methods. ¹H NMR (500 MHz, CDCl₃): δ 0.88 (9H, t, J=7.18 Hz), 1.25 (50H, m), 1.63 (16H, m), 2.00 (4H, q, J=6.20 Hz), 2.25 (4H, m), 2.38 (6H, m), 3.24 (2H, m), 4.15 (2H, t, J=6.70 Hz), 4.34 (1H, dd, J=11.58, 2.77 Hz), 4.44 (1H, dd, J=11.58, 4.31 Hz), 4.80 (1H, m), 4.86 (1H, i, J=6.00 Hz), 5.34 (3H, m), 6.42 (1H, d, J=7.62 Hz). MS [ESI]: m/z: [M+H] calc 904.8 obs. 904.6.

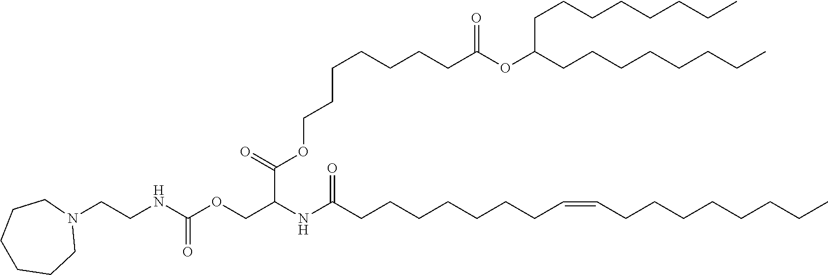 [SMILES: CCCCCCCC/C=C\CCCCCCCC(=O)NC(COC(=O)NCCN1CCCCCC1)C(=O)OCCCCCCCC(=O)OC(CCCCCCCC)CCCCCCCC]

Heptadecan-9-yl 8-((O-((2-(azepan-1-yl)ethyl)carbamoyl)-N-oleoylseryl)oxy)octanoate was synthesized according to representative synthetic scheme 1 and the corresponding methods. ¹H NMR (500 MHz, CDCl₃): δ 0.88 (9H, t, J=7.13 Hz), 1.26 (50H, m), 1.50 (4H, m), 1.63 (14H, m), 2.01 (4H, q, J=6.11 Hz), 2.25 (4H, m), 2.60 (6H, m), 3.20 (2H, m), 4.15 (2H, t, J=6.79 Hz), 4.34 (1H, dd, J=11.74, 3.20 Hz), 4.45 (1H, dd, J=11.74, 4.21 Hz), 4.79 (1H, m), 4.86 (1H, i, J=6.52 Hz), 5.34 (3H, m), 6.42 (1H, m). MS [ESI]: m/z: [M+H] calc 918.8 obs. 918.6.

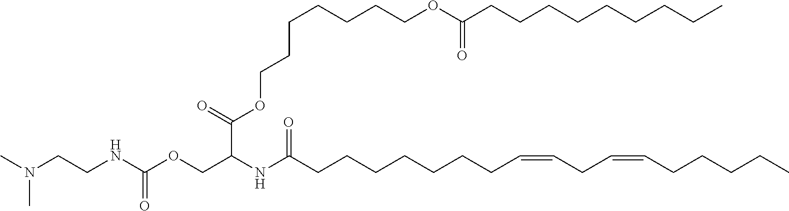 [SMILES: CCCCC/C=C\C/C=C\CCCCCCCC(=O)NC(COC(=O)NCCN(C)C)C(=O)OCCCCCCCOC(=O)CCCCCCCCC]

2-Methyl-9-((9Z,12Z)-octadeca-9,12-dienamido)-6,10-dioxo-7,11-dioxa-2,5-diazaoctadecan-18-yl decanoate was synthesized according to representative synthetic scheme 1 and the corresponding methods. ¹H NMR (400 MHz, CDCl₃): δ 0.88 (6H, m), 1.28 (32H, m), 1.63 (8H, m), 2.05 (4H, q, J=6.83 Hz), 2.23 (10H, m), 2.40 (2H, t, J=5.59 Hz), 2.77 (2H, t, J=6.52 Hz), 3.24 (2H, m), 4.05 (2H, t, J=6.70 Hz), 4.15 (2H, t, J=6.70 Hz), 4.34 (1H, dd, J=11.37, 3.30 Hz), 4.44 (1H, dd, J=11.37, 4.24 Hz), 4.80 (1H, m), 5.34 (5H, m), 6.39 (1H, d, J=8.10 Hz). MS [ESI]: m/z: [M+H] calc 750.6 obs. 750.8.

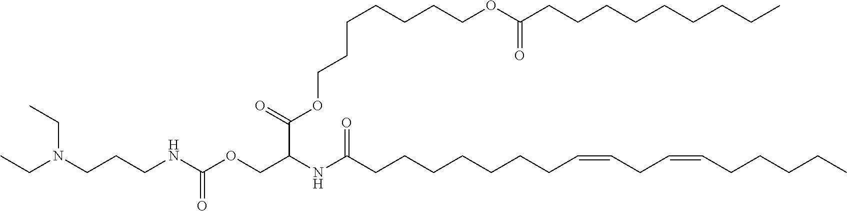 [SMILES: CCCCC/C=C\C/C=C\CCCCCCCC(=O)NC(COC(=O)NCCCN(CC)CC)C(=O)OCCCCCCCOC(=O)CCCCCCCCC]

3-Ethyl-11-((9Z,12Z)-octadeca-9,12-dienamido)-8,12-dioxo-9,13-dioxa-3,7-diazaicosan-20-yl decanoate was synthesized according to representative synthetic scheme 1 and the corresponding methods. ¹H NMR (400 MHz, CDCl₃): δ 0.88 (6H, m), 1.02 (6H, t, J=7.13 Hz), 1.30 (32H, m), 1.62 (10H, m), 2.04 (4H, q, J=7.26 Hz), 2.23 (2H, t, J=8.01 Hz), 2.29 (2H, t, J=7.61 Hz), 2.48 (6H, m), 2.77 (2H, t, J=6.42 Hz), 3.24 (2H, m), 4.05 (2H, t, J=6.70 Hz), 4.13 (2H, t, J=6.79 Hz), 4.33 (1H, dd, J=11.55, 3.17 Hz), 4.42 (1H, dd, J=11.55, 4.61 Hz), 4.77 (1H, m), 5.34 (4H, m), 6.40 (2H, m). MS [ESI]: m/z: [M+H] calc 792.6 obs. 792.8.

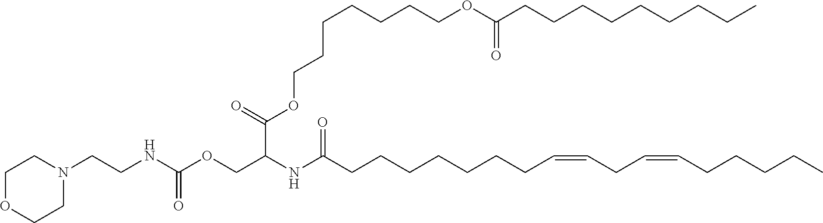 [SMILES: CCCCC/C=C\C/C=C\CCCCCCCC(=O)NC(COC(=O)NCCN1CCOCC1)C(=O)OCCCCCCCOC(=O)CCCCCCCCC]

7-((O-((2-Morpholinoethyl)carbamoyl)-N-((9Z,12Z)-octadeca-9,12-dienoyl)seryl)oxy)heptyl decanoate was synthesized according to representative synthetic scheme 1 and the corresponding methods. ¹H NMR (400 MHz, CDCl₃): δ 0.88 (6H, m), 1.29 (32H, m), 1.60 (8H, m), 2.04 (4H, q, J=7.15 Hz), 2.29 (4H, m), 2.44 (6H, m), 2.77 (2H, t, J=6.56 Hz), 3.26 (2H, m), 3.70 (4H, t, J=4.61 Hz), 4.05 (2H, t, J=6.70 Hz), 4.15 (2H, m), 4.35 (1H, dd, J=11.32, 3.60 Hz), 4.45 (1H, dd, J=11.32, 4.33 Hz), 4.80 (1H, m), 5.20 (1H, m), 5.34 (4H, m), 6.37 (1H, d, J=7.86 Hz). MS [ESI]: m/z: [M+H] calc 792.6 obs. 792.8.

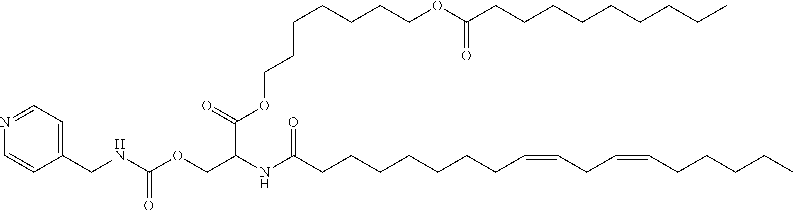 [SMILES: CCCCC/C=C\C/C=C\CCCCCCCC(=O)NC(COC(=O)NCc1ccncc1)C(=O)OCCCCCCCOC(=O)CCCCCCCCC]

7-((N-((9Z,12Z)-Octadeca-9,12-dienoyl)-O-((pyridin-4-ylmethyl)carbamoyl)seryl)oxy)heptyl decanoate was synthesized according to representative synthetic scheme 1 and the corresponding methods. ¹H NMR (400 MHz, CDCl₃): δ 0.88 (6H, m), 1.30 (32H, m), 1.64 (8H, m), 2.04 (4H, q, J=6.86 Hz), 2.28 (4H, m), 2.76 (2H, t, J=6.21 Hz), 4.04 (2H, t, J=6.70 Hz), 4.15 (2H, m), 4.37 (2H, d, J=6.05 Hz), 4.42 (1H, dd, J=11.54, 3.32 Hz), 4.49 (1H, dd, J=11.54, 4.07 Hz), 4.83 (1H, m), 5.33 (5H, m), 6.32 (1H, d, J=7.77 Hz), 7.19 (2H, d, J=5.12 Hz), 8.57 (2H, d, J=5.12 Hz). MS [ESI]: m/z: [M+H] calc 770.6 obs. 770.6.

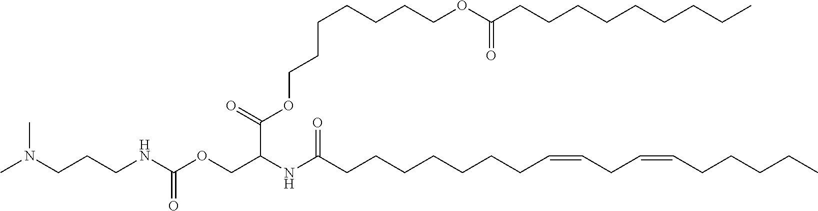 [SMILES: CCCCC/C=C\C/C=C\CCCCCCCC(=O)NC(COC(=O)NCCCN(C)C)C(=O)OCCCCCCCOC(=O)CCCCCCCCC]

2-Methyl-10-((9Z,12Z)-octadeca-9,12-dienamido)-7,11-dioxo-8,12-dioxa-2,6-diazanonadecan-19-yl decanoate was synthesized according to representative synthetic scheme 1 and the corresponding methods. ¹H NMR (400 MHz, CDCl₃): δ 0.88 (6H, m), 1.29 (32H, m), 1.63 (10H, m), 2.04 (4H, q, J=6.85 Hz), 2.22 (8H, m), 2.29 (2H, t, J=7.67 Hz), 2.35 (2H, t, J=6.57 Hz), 2.77 (2H, t, J=6.52 Hz), 3.23 (2H, m), 4.05 (2H, t, J=6.70 Hz), 4.14 (2H, t, J=6.69 Hz), 4.33 (1H, dd, J=11.33, 3.21 Hz), 4.43 (1H, dd, J=11.33, 4.52 Hz), 4.78 (1H, m), 5.34 (4H, m), 5.74 (1H, t, J=6.01 Hz), 6.43 (1H, d, J=7.79 Hz). MS [ESI]: m/z: [M+H] calc 764.6 obs. 764.8.

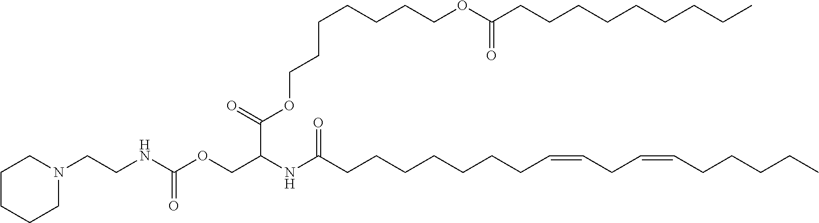 [SMILES: CCCCC/C=C\C/C=C\CCCCCCCC(=O)NC(COC(=O)NCCN1CCCCC1)C(=O)OCCCCCCCOC(=O)CCCCCCCCC]

7-((N-((9Z,12Z)-Octadeca-9,12-dienoyl)-O-((2-(piperidin-1-yl)ethyl)carbamoyl)seryl)oxy)heptyl decanoate was synthesized according to representative synthetic scheme 1 and the corresponding methods. 1H NMR (400 MHz, CDCl₃): δ 0.88 (6H, m), 1.29 (34H, m), 1.62 (12H, m), 2.04 (4H, q, J=7.03 Hz), 2.29 (4H, m), 2.38 (6H, m), 2.77 (2H, t, J=6.39 Hz), 3.24 (2H, m), 4.05 (2H, t, J=6.70 Hz), 4.15 (2H, t, J=6.83 Hz), 4.34 (1H, dd, J=11.90, 3.40 Hz), 4.45 (1H, dd, J=11.90, 4.42 Hz), 4.79 (1H, m), 5.34 (5H, m), 6.42 (1H, d, J=7.93 Hz). MS [ESI]: m/z: [M+H] calc 790.6 obs. 790.7.

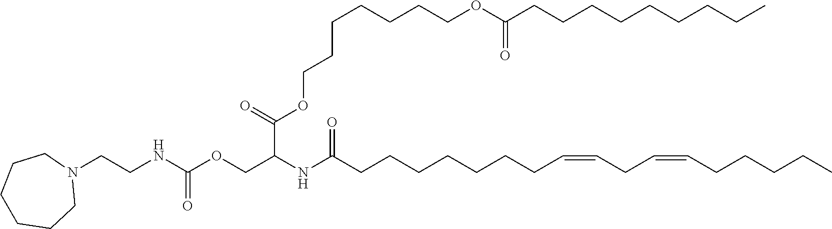 [SMILES: CCCCC/C=C\C/C=C\CCCCCCCC(=O)NC(COC(=O)NCCN1CCCCCC1)C(=O)OCCCCCCCOC(=O)CCCCCCCCC]

7-((O-((2-(Azepan-1-yl)ethyl)carbamoyl)-N-((9Z,12Z)-octadeca-9,12-dienoyl)seryl)oxy)heptyl decanoate was synthesized according to representative synthetic scheme 1 and the corresponding methods. ¹H NMR (400 MHz, CDCl₃): δ 0.88 (6H, m), 1.30 (32H, m), 1.60 (16H, m), 2.05 (4H, q, J=7.00 Hz), 2.29 (4H, m), 2.62 (6H, m), 2.77 (2H, t, J=6.59 Hz), 3.20 (2H, m), 4.05 (2H, t, J=6.70 Hz), 4.15 (2H, t, J=6.88 Hz), 4.34 (1H, dd, J=11.92, 3.32 Hz), 4.45 (1H, dd, J=11.92, 4.37 Hz), 4.80 (1H, m), 5.34 (5H, m), 6.42 (1H, d, J=7.69 Hz). MS [ESI]: m/z: [M+H] calc 804.6 obs. 804.7.

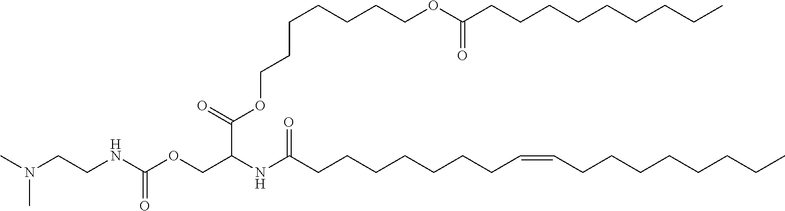 [SMILES: CCCCCCCC/C=C\CCCCCCCC(=O)NC(COC(=O)NCCN(C)C)C(=O)OCCCCCCCOC(=O)CCCCCCCCC]

2-Methyl-9-oleamido-6,10-dioxo-7,11-dioxa-2,5-diazaoctadecan-18-yl decanoate was synthesized according to representative synthetic scheme 1 and the corresponding methods. ¹H NMR (400 MHz, CDCl₃): δ 0.88 (6H, m), 1.28 (38H, m), 1.63 (9H, m), 2.00 (4H, m), 2.22 (10H, s), 2.39 (2H, t, J=6.33 Hz), 3.24 (1H, dd, J=5.47, 3.07 Hz), 4.05 (2H, t, J=6.71 Hz), 4.15 (2H, t, J=6.71 Hz), 4.35 (1H, m), 4.41-4.47 (1H, m), 4.77-4.82 (1H, 4.80 (d, J=15.08 Hz), 4.80 (t, J=4.36 Hz)), 5.27 (1H, m), 5.34 (2H, m), 6.39 (1H, m). MS [ESI]: m/z: [M+H] calc 752.6 obs. 752.3.

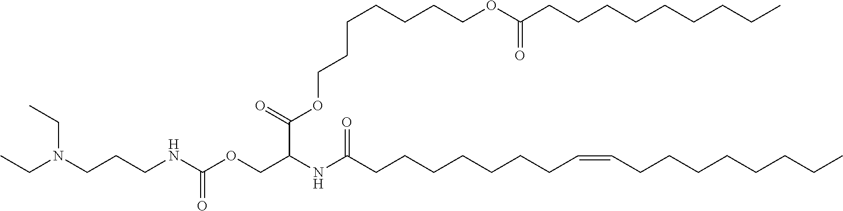 [SMILES: CCCCCCCC/C=C\CCCCCCCC(=O)NC(COC(=O)NCCCN(CC)CC)C(=O)OCCCCCCCOC(=O)CCCCCCCCC]

3-Ethyl-11-oleamido-8,12-dioxo-9,13-dioxa-3,7-diazaicosan-20-yl decanoate was synthesized according to representative synthetic scheme 1 and the corresponding methods. ¹H NMR (400 MHz, CDCl₃): δ 0.88 (6H, m), 1.02 (6H, t, J=7.13 Hz), 1.28 (38H, m), 1.54-1.69 (11H, m), 2.00 (4H, m), 2.26 (4H, m), 2.49 (6H, m), 3.25 (2H, m), 4.05 (2H, t, J=6.71 Hz), 4.13 (2H, t, J=6.75 Hz), 4.41 (2H, m), 4.74-4.82 (1H, m), 5.34 (2H, m), 6.37-6.45 (1H, m). MS [ESI]: m/z: [M+H] calc 794.7 obs. 794.3.

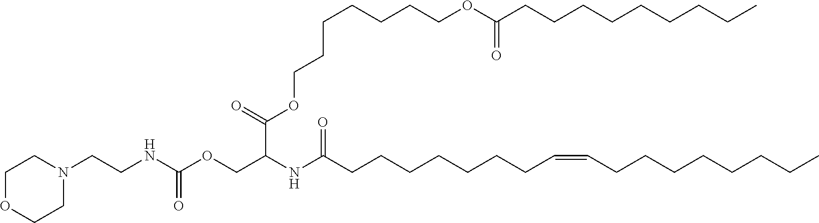 [SMILES: CCCCCCCC/C=C\CCCCCCCC(=O)NC(COC(=O)NCCN1CCOCC1)C(=O)OCCCCCCCOC(=O)CCCCCCCCC]

7-((O-((2-Morpholinoethyl)carbamoyl)-N-oleoylseryl)oxy)heptyl decanoate was synthesized according to representative synthetic scheme 1 and the corresponding methods. ¹H NMR (400 MHz, CDCl₃): δ 0.88 (6H, m), 1.26 (38H, m), 1.61 (9H, m), 2.00 (4H, m), 2.27 (4H, m), 2.44 (6H, m), 3.27 (1H, m), 3.70 (4H, t, J=4.38 Hz), 4.05 (2H, t, J=6.72 Hz), 4.15 (2H, td, J=6.70, 3.48 Hz), 4.32-4.38 (1H, m), 4.45 (1H, br s), 4.80 (1H, t, J=3.88 Hz), 5.20 (1H, br s), 5.34 (2H, m), 6.38 (1H, s). MS [ESI]: m/z: [M+H] calc 794.6 obs. 794.8.

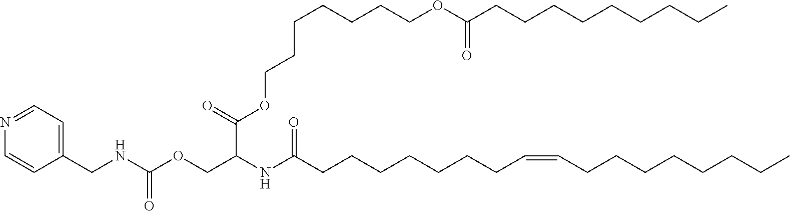 [SMILES: CCCCCCCC/C=C\CCCCCCCC(=O)NC(COC(=O)NCc1ccncc1)C(=O)OCCCCCCCOC(=O)CCCCCCCCC]

7-((N-Oleoyl-O-((pyridin-4-ylmethyl)carbamoyl)seryl)oxy)heptyl decanoate was synthesized according to representative synthetic scheme 1 and the corresponding methods. ¹H NMR (400 MHz, CDCl₃): δ 0.88 (6H, t, J=6.73 Hz), 1.21-1.38 (38H, m), 1.58-1.68 (10H, m), 1.94-2.03 (4H, m), 2.27 (4H, m), 4.05 (2H, t, J=6.73 Hz), 4.15 (2H, m), 4.37 (2H, d, J=6.66 Hz), 4.45 (1H, m), 4.83 (1H, m), 5.19-5.25 (1H, m), 5.34 (2H, m), 6.31 (1H, d, J=8.04 Hz), 7.18 (2H, d, J=6.04 Hz), 8.57 (2H, dd, J=4.25, 1.35 Hz). MS [ESI]: m/z: [M+H] calc 772.6 obs. 772.6.

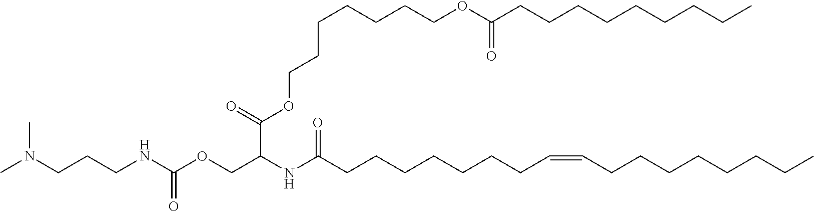 [SMILES: CCCCCCCC/C=C\CCCCCCCC(=O)NC(COC(=O)NCCCN(C)C)C(=O)OCCCCCCCOC(=O)CCCCCCCCC]

2-Methyl-10-oleamido-7,11-dioxo-8,12-dioxa-2,6-diazanonadecan-19-yl decanoate was synthesized according to representative synthetic scheme 1 and the corresponding methods. ¹H NMR (400 MHz, CDCl₃): δ 0.87 (6H, m), 1.28 (38H, m), 1.63 (10H, m), 2.00 (4H, m), 2.21 (12H, m), 3.24 (2H, q, J=6.08 Hz), 4.05 (2H, t, J=6.71 Hz), 4.14 (2H, t, J=6.71 Hz), 4.30-4.36 (1H, m), 4.42 (1H, m), 4.77 (1H, m), 5.34 (2H, m), 5.71-5.78 (1H, m), 6.40-6.46 (1H, m). MS [ESI]: m/z: [M+H] calc 766.6 obs. 766.3.

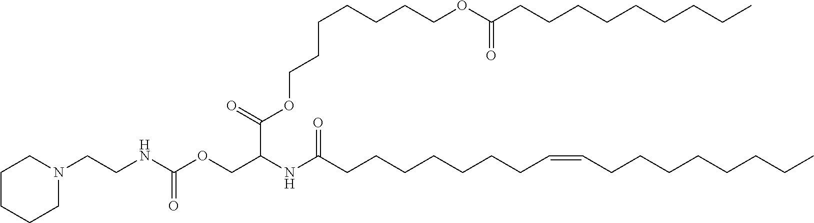 [SMILES: CCCCCCCC/C=C\CCCCCCCC(=O)NC(COC(=O)NCCN1CCCCC1)C(=O)OCCCCCCCOC(=O)CCCCCCCCC]

7-((N-Oleoyl-O-((2-(piperidin-1-yl)ethyl)carbamoyl)seryl)oxy)heptyl decanoate was synthesized according to representative synthetic scheme 1 and the corresponding methods. ¹H NMR (400 MHz, CDCl₃): δ 0.88 (6H, m), 1.27 (40H, m), 1.58-1.69 (11H, m), 1.95-2.05 (4H, m), 2.29 (5H, m), 4.05 (2H, t, J=6.75 Hz), 4.15 (2H, br s), 5.32-5.36 (2H, m), 2.34-2.45 (5H, m), 3.21-3.28 (2H, m), 4.30-4.36 (1H, m), 4.42-4.49 (1H, m), 4.75-4.84 (2H, m), 6.38-645(1H, m). MS [ESI]: m/z: [M+H] calc 292.6 ohs. 792.4.

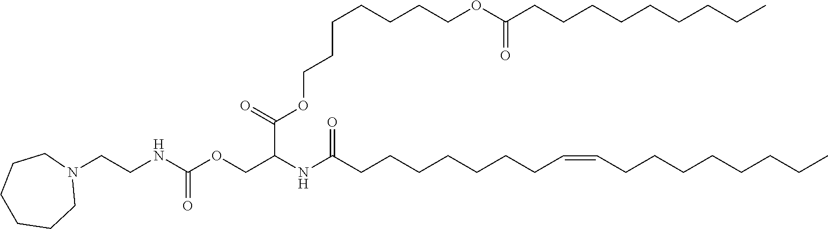 [SMILES: CCCCCCCC/C=C\CCCCCCCC(=O)NC(COC(=O)NCCN1CCCCCC1)C(=O)OCCCCCCCOC(=O)CCCCCCCCC]

7-((O-((2-(Azepan-1-yl)ethyl)carbamoyl)-N-oleoylseryl)oxy)heptyl decanoate was synthesized according to representative synthetic scheme 1 and the corresponding methods. ¹H NMR (400 MHz, CDCl₃): δ 0.88 (6H, m), 1.28 (38H, m), 1.60 (19H, m), 1.97-2.03 (3H, m), 2.27 (4H, m), 2.61 (5H, m), 3.17-3.23 (1H, m), 4.05 (2H, t, J=6.71 Hz), 4.15 (2H, t, J=6.76 Hz), 4.31-4.36 (1H, m), 4.42-4.48 (1H, 4.44 (d, J=4.27 Hz), 4.47 (d, J=4.36 Hz)), 4.80 (1H, m), 5.34 (2H, m), 6.38-6.46 (1H, m). MS [ESI]: m/z: [M+H] calc 806.7 obs. 806.3.

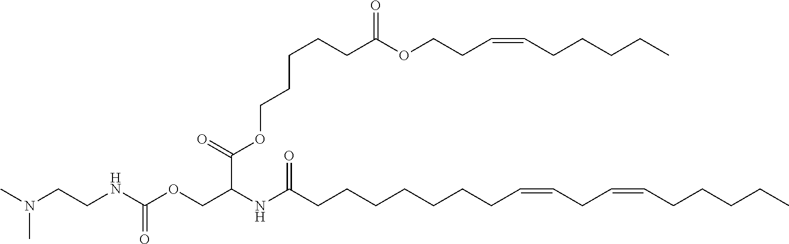 [SMILES: CCCCC/C=C\C/C=C\CCCCCCCC(=O)NC(COC(=O)NCCN(C)C)C(=O)OCCCCCC(=O)OCC/C=C\CCCCC]

(Z)-Non-3-en-1-yl 6-((O-((2-(dimethylamino)ethyl)carbamoyl)-N-((9Z,12Z)-octadeca-9,12-dienoyl)seryl)oxy)hexanoate was synthesized according to representative synthetic scheme 1 and the corresponding methods. ¹H NMR (400 MHz, CDCl₃): δ 0.89 (6H, m), 1.31 (22H, m), 1.65 (7H, m), 2.04 (6H, m), 2.22 (14H, m), 2.77 (2H, t, J=5.99 Hz), 3.25 (2H, d, J=1.62 Hz), 4.06 (2H, t, J=6.96 Hz), 4.16 (2H, td, J=6.57, 1.86 Hz), 4.31-4.37 (1H, m), 4.41-4.46 (1H, 4.42 (d, J=3.89 Hz), 4.45 (d, J=3.96 Hz)), 4.79 (1H, m), 5.34 (5H, m), 5.49 (1H, m), 6.43 (1H, m). MS [ESI]: m/z: [M+H] calc 720.6 obs. 720.7.

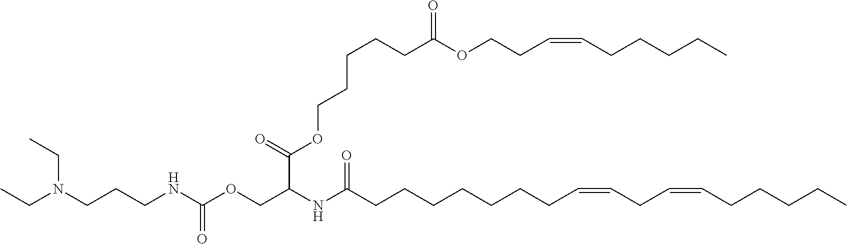 [SMILES: CCCCC/C=C\C/C=C\CCCCCCCC(=O)NC(COC(=O)NCCCN(CC)CC)C(=O)OCCCCCC(=O)OCC/C=C\CCCCC]

(Z)-Non-3-en-1-yl 6-((O-((3-(diethylamino)propyl)carbamoyl)-N-((9Z,12Z)-octadeca-9,12-dienoyl)seryl)oxy)hexanoate was synthesized according to representative synthetic scheme 1 and the corresponding methods. ¹H NMR (400 MHz, CDCl₃): δ 0.89 (6H, m), 1.02 (6H, t, J=7.13 Hz), 1.31 (22H, m), 1.62 (8H, m), 2.04 (6H, m), 2.23 (2H, t, J=7.66 Hz), 2.30 (4H, m), 2.48 (6H, m), 2.77 (2H, t, J=5.99 Hz), 3.22 (2H, m), 4.06 (2H, t, J=6.96 Hz), 4.14 (2H, t, J=6.49 Hz), 4.30-4.44 (2H, m), 4.76 (1H, m), 5.35 (5H, m), 5.49 (1H, m), 6.36-6.47 (2H, m). MS [ESI]: m/z: [M+H] calc 762.6 obs. 762.3.

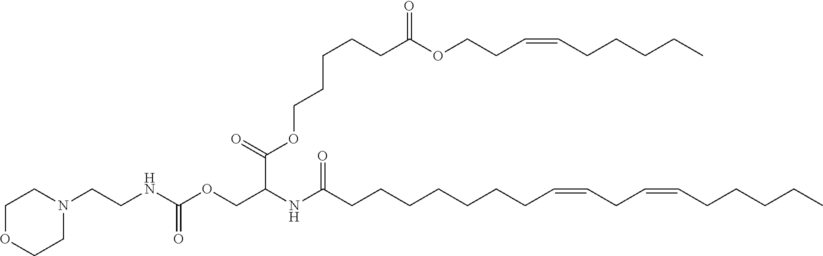 [SMILES: CCCCC/C=C\C/C=C\CCCCCCCC(=O)NC(COC(=O)NCCN1CCOCC1)C(=O)OCCCCCC(=O)OCC/C=C\CCCCC]

(Z)-Non-3-en-1-yl 6-((O-((2-morpholinoethyl)carbamoyl)-N-((9Z,12Z)-octadeca-9,12-dienoyl)seryl)oxy)hexanoate was synthesized according to representative synthetic scheme 1 and the corresponding methods. ¹H NMR (400 MHz, CDCl₃): δ 0.89 (6H, t, J=6.85 Hz), 1.31 (23H, m), 1.61-1.71 (6H, m), 2.04 (6H, m), 2.24 (2H, t, J=7.51 Hz), 2.31 (2H, t, J=7.43 Hz), 2.37 (2H, q, J=6.99 Hz), 2.41-2.50 (6H, m) 2.77 (2H, m), 3.28 (1H, m), 3.70 (4H, t, J=4.87 Hz), 4.06 (2H, t, J=6.95 Hz), 4.17 (2H, t, J=6.30 Hz), 4.33-4.39 (1H, m), 4.41-4.47 (1H, m), 4.81 (1H, m), 5.35 (5H, m), 5.47-5.53 (1H, m), 6.40 (1H, d, J=7.70 Hz). MS [ESI]: m/z: [M+H] calc 762.6 obs. 762.5.

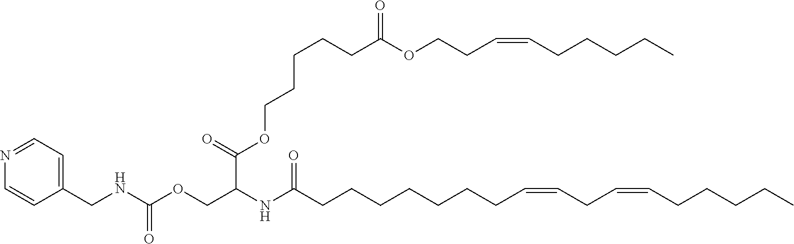 [SMILES: CCCCC/C=C\C/C=C\CCCCCCCC(=O)NC(COC(=O)NCc1ccncc1)C(=O)OCCCCCC(=O)OCC/C=C\CCCCC]

(Z)-Non-3-en-1-yl 6-((N-((9Z,12Z)-octadeca-9,12-dienoyl)-O-((pyridin-4-ylmethyl)carbamoyl)seryl)oxy)hexanoate was synthesized according to representative synthetic scheme 1 and the corresponding methods. ¹H NMR (400 MHz, CDCl₃): δ 0.89 (6H, t, J=6.79 Hz), 1.31 (21H, m), 1.62 (1H, m), 2.03 (6H, m), 2.30 (6H, m), 2.76 (2H, t, J=6.04 Hz), 4.03 (2H, t, J=6.90 Hz), 4.15 (2H, m), 4.37 (2H, d, J=6.16 Hz), 4.45 (2H, d, J=3.48 Hz), 4.82 (1H, t, J=3.84 Hz), 5.34 (5H, m), 5.51 (2H, m), 6.38 (1H, m), 7.19 (2H, m), 8.56 (2H, d, J=4.20 Hz). MS [ESI]: m/z: [M+H] calc 740.5 obs. 740.5.

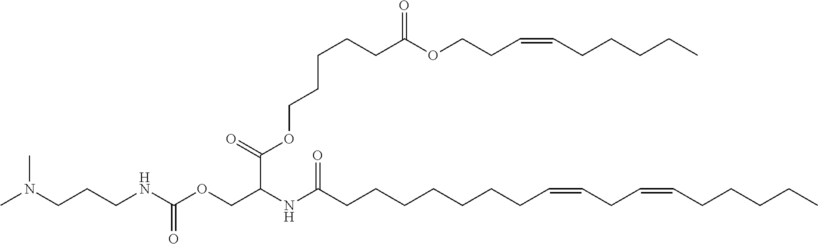 [SMILES: CCCCC/C=C\C/C=C\CCCCCCCC(=O)NC(COC(=O)NCCCN(C)C)C(=O)OCCCCCC(=O)OCC/C=C\CCCCC]

(Z)-Non-3-en-1-yl 6-((O-((3-(dimethylamino)propyl)carbamoyl)-N-((9Z,12Z)-octadeca-9,12-dienoyl)seryl)oxy)hexanoate was synthesized according to representative synthetic scheme 1 and the corresponding methods. ¹H NMR (400 MHz, CDCl₃): δ 0.89 (6H, t, J=6.75 Hz), 1.31 (19H, m), 1.64 (10H, m), 2.04 (6H, m), 2.21 (8H, m), 2.31 (6H, m), 2.77 (2H, t, J=6.03 Hz), 3.23 (1H, m), 3.64 (2H, s), 4.06 (2H, t, J=6.95 Hz), 4.13-4.19 (2H, m), 4.34 (1H, br s), 4.40-4.47 (1H, m), 4.77 (1Hm), 5.34 (5H, m), 5.46-5.54 (1H, m), 5.76-5.83 (1H, m), 6.42-6.49 (1H, m). MS [ESI]: m/z: [M+H] calc 734.6 obs. 734.7.

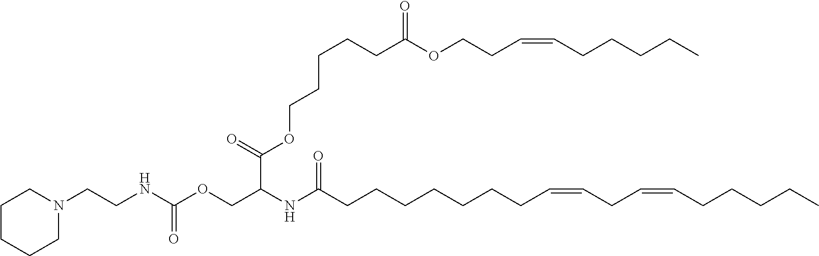 [SMILES: CCCCC/C=C\C/C=C\CCCCCCCC(=O)NC(COC(=O)NCCN1CCCCC1)C(=O)OCCCCCC(=O)OCC/C=C\CCCCC]

(Z)-Non-3-en-1-yl 6-((N-((9Z,12Z)-octadeca-9,12-dienoyl)-O-((2-(piperidin-1-yl)ethyl)carbamoyl)seryl)oxy)hexanoate was synthesized according to representative synthetic scheme 1 and the corresponding methods. ¹H NMR (400 MHz, CDCl₃): δ 0.89 (6H, t, J=6.62 Hz), 1.31 (23H, m), 1.63 (12H, m), 2.04 (6H, m), 2.37 (11H, m), 2.77 (2H, t, J=5.99 Hz), 3.25 (1H, m), 4.06 (2H, t, J=6.96 Hz), 4.16 (2H, m), 4.43 (1H, d, J=4.08 Hz), 5.34 (5H, m), 5.50 (1H, d, J=10.81 Hz), 4.31-4.37 (1H, m), 4.75-4.83 (1H, m), 6.45 (1H, d, J=7.86 Hz). MS [ESI]: m/z: [M+H] calc 760.6 obs. 760.6.

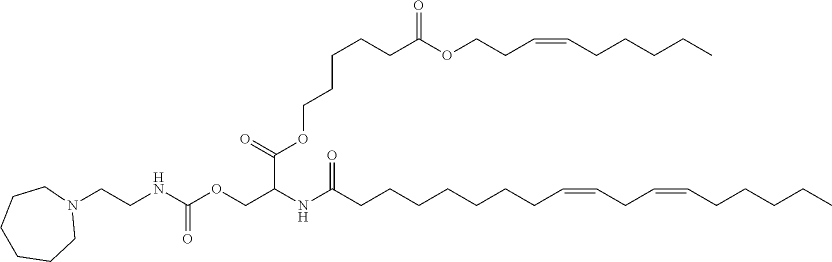 [SMILES: CCCCC/C=C\C/C=C\CCCCCCCC(=O)NC(COC(=O)NCCN1CCCCCC1)C(=O)OCCCCCC(=O)OCC/C=C\CCCCC]

(Z)-Non-3-en-1-yl 6-((O-((2-(azepan-1-yl)ethyl)carbamoyl)-N-((9Z,12Z)-octadeca-9,12-dienoyl)seryl)oxy)hexanoate was synthesized according to representative synthetic scheme 1 and the corresponding methods. ¹H NMR (400 MHz, CDCl₃): δ 0.89 (6H, t, J=6.84 Hz), 1.31 (22H, m), 1.62 (15H, m), 2.04 (6H, m), 2.24 (2H, t, J=7.39 Hz), 2.30 (2H, t, J=7.43 Hz), 2.37 (2H, q, J=7.00 Hz), 2.62 (6H, m), 2.77 (2H, t, J=5.92 Hz), 3.20 (1H, m), 4.06 (2H, t, J=6.96 Hz), 4.16 (2H, q, J=6.78 Hz), 4.31-4.38 (1H, m), 4.44 (1H, m), 4.79 (1H, m), 5.35 (5H, m), 5.49 (1H, m), 6.42-6.47 (1H, m). MS [ESI]: m/z: [M+H] calc 774.6 obs. 774.7.

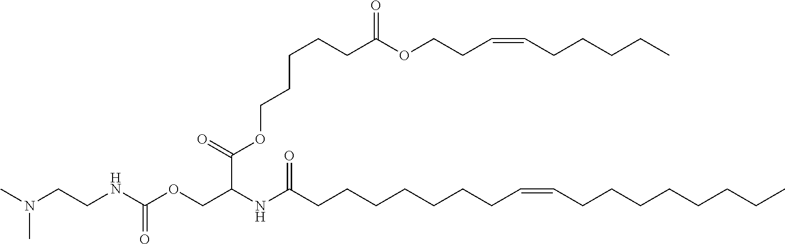 [SMILES: CCCCC/C=C\CCOC(=O)CCCCCOC(=O)C(COC(=O)NCCN(C)C)NC(=O)CCCCCCC/C=C\CCCCCCCC]

(Z)-Non-3-en-1-yl 6-((O-((2-(dimethylamino)ethyl)carbamoyl)-N-oleoylseryl)oxy)hexanoate was synthesized according to representative synthetic scheme 1 and the corresponding methods. ¹H NMR (400 MHz, CDCl₃): δ 0.88 (6H, td, J=6.85, 3.47 Hz), 1.28 (28H, m), 1.65 (6H, m), 2.01 (6H, m), 2.43 (12H, m), 2.57-2.67 (2H, m), 3.34 (2H, m), 4.06 (2H, t, J=6.95 Hz), 4.14 (2H, t, J=6.61 Hz), 4.32 (1H, dd, J=11.29, 2.95 Hz), 4.47 (1H, m), 4.80 (1H, m), 5.34 (3H, m), 5.50 (1H, m), 5.72-5.85 (1H, m) 6.72-6.84 (1H, m). MS [ESI]: m/z: [M+H] calc 722.6 obs. 722.4.

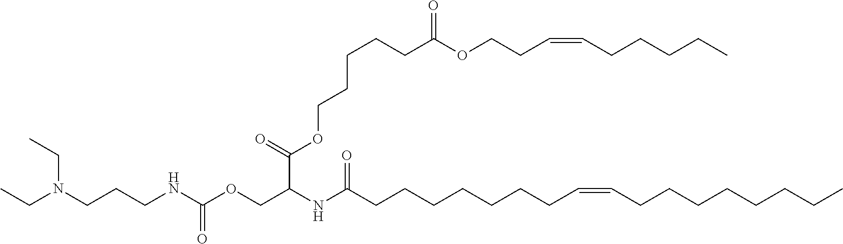 [SMILES: CCCCC/C=C\CCOC(=O)CCCCCOC(=O)C(COC(=O)NCCCN(CC)CC)NC(=O)CCCCCCC/C=C\CCCCCCCC]

(Z)-Non-3-en-1-yl 6-((O-((3-(diethylamino)propyl)carbamoyl)-N-oleoylseryl)oxy)hexanoate was synthesized according to representative synthetic scheme 1 and the corresponding methods. ¹H NMR (400 MHz, CDCl₃): δ 0.88 (6H, m), 1.11 (6H, t, J=7.08 Hz), 1.24-1.37 (27H, m), 1.64 (8H, m), 1.97-2.05 (6H, m), 2.24 (2H, t, J=7.59 Hz), 2.30 (3H, t, J=7.35 Hz), 2.36 (2H, q, J=7.43 Hz), 2.59-2.70 (6H, m), 3.26 (2H, q, J=5.80 Hz), 4.06 (2H, t, J=6.95 Hz), 4.14 (2H, t, J=6.58 Hz), 4.30 (1H, m), 4.41 (1H, m), 4.77 (1H, m), 5.34 (3H, m), 5.49 (1H, m), 6.31 (1H, m), 6.53 (1H, m). MS [ESI]: m/z: [M+H] calc 764.6 obs. 764.5.

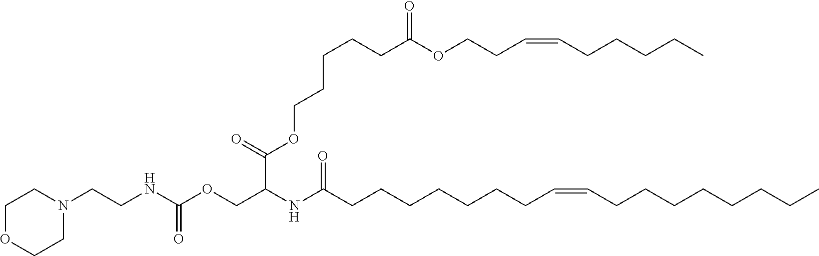 [SMILES: CCCCC/C=C\CCOC(=O)CCCCCOC(=O)C(COC(=O)NCCN1CCOCC1)NC(=O)CCCCCCC/C=C\CCCCCCCC]

(Z)-Non-3-en-1-yl 6-((O-((2-morpholinoethyl)carbamoyl)-N-oleoylseryl)oxy)hexanoate was synthesized according to representative synthetic scheme 1 and the corresponding methods. ¹H NMR (400 MHz, CDCl₃): δ 0.88 (6H, m), 1.28 (30H, m), 1.65 (5H, m), 2.01 (6H, m), 2.31 (7H, m), 2.66-2.84 (4H, m), 3.38 (2H, m), 3.79 (1H, m), 3.87 (3H, m), 4.06 (2H, t, J=6.95 Hz), 4.14 (2H, t, J=6.61 Hz), 4.28-4.36 (1H, m), 4.45-4.53 (1H, m), 4.80 (1H, m), 5.34 (3H, m), 5.49 (1H, m), 5.79-5.90 (1H, m), 6.73-6.81 (1H, m). MS [ESI]: m/z: [M+H] calc 764.6 obs. 764.3.

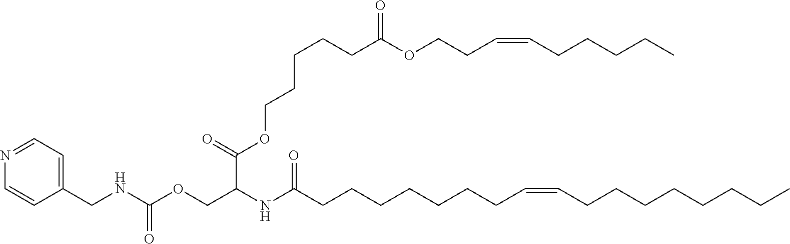 [SMILES: CCCCC/C=C\CCOC(=O)CCCCCOC(=O)C(COC(=O)NCc1ccncc1)NC(=O)CCCCCCC/C=C\CCCCCCCC]

(Z)-Non-3-en-1-yl 6-((N-oleoyl-O-((pyridin-4-ylmethyl)carbamoyl)seryl)oxy)hexanoate was synthesized according to representative synthetic scheme 1 and the corresponding methods. ¹H NMR (400 MHz, CDCl₃): δ 0.85-0.91 (6H, m), 1.29 (29H, m), 1.64 (6H, br s), 1.94-2.05 (6H, m), 2.23 (2H, t, J=7.52 Hz), 2.32 (4H, m), 4.03 (2H, t, J=6.86 Hz), 4.13 (1H, m), 4.18-4.25 (1H, m), 4.45 (4H, m), 4.84 (1H, m), 5.34 (3H, m), 5.49 (1H, m), 5.99 (1H, m), 6.43-6.50 (1H, m), 7.53 (2H, d, J=5.64 Hz), 8.61 (2H, dd, J=4.53, 1.46 Hz). MS [ESI]: m/z: [M+H] calc 742.5 obs. 742.3.

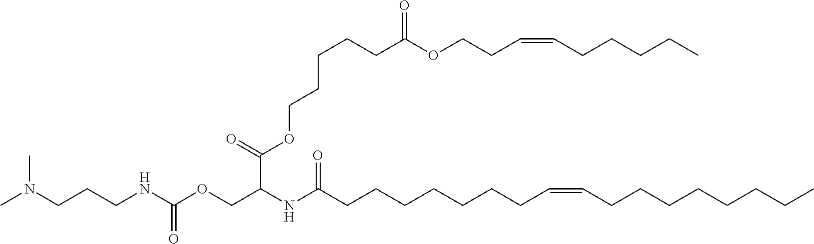 [SMILES: CCCCC/C=C\CCOC(=O)CCCCCOC(=O)C(COC(=O)NCCCN(C)C)NC(=O)CCCCCCC/C=C\CCCCCCCC]

(Z)-Non-3-en-1-yl 6-((O-((3-(dimethylamino)propyl)carbamoyl)-N-oleoylseryl)oxy)hexanoate was synthesized according to representative synthetic scheme 1 and the corresponding methods. ¹H NMR (400 MHz, CDCl₃): δ 0.88 (6H, m), 1.31 (29H, m), 1.59-1.71 (6H, m), 1.96-2.03 (4H, m), 2.26 (2H, t, J=7.61 Hz), 2.31 (2H, t, J=7.45 Hz), 2.47 (6H, s), 1.79-1.87 (2H, m), 2.37 (2H, q, J=7.25 Hz), 2.63-2.69 (2H, m), 3.29 (2H, m), 4.06 (2H, t, J=6.94 Hz), 4.15 (2H, td, J=6.86, 2.77 Hz), 4.30-4.35 (1H, m), 4.42-4.47 (1H, m), 4.77-4.82 (1H, m), 5.34 (3H, m), 5.49 (1H, m), 5.87-5.94 (1H, m), 6.55-6.60 (1H, m). MS [ESI]: m/z: [M+H] calc 736.6 obs. 736.3.

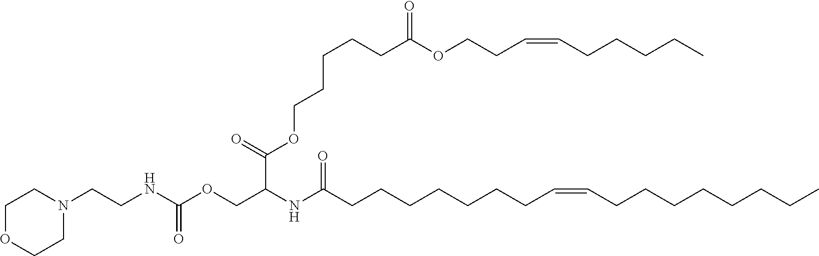 [SMILES: CCCCC/C=C\CCOC(=O)CCCCCOC(=O)C(COC(=O)NCCN1CCOCC1)NC(=O)CCCCCCC/C=C\CCCCCCCC]

(Z)-Non-3-en-1-yl 6-((N-oleoyl-O-((2-(piperidin-1-yl)ethyl)carbamoyl)seryl)oxy)hexanoate was synthesized according to representative synthetic scheme 1 and the corresponding methods. ¹H NMR (400 MHz, CDCl₃): δ 0.88 (6H, m), 1.26 (35H, m), 1.57-1.71 (9H, m), 1.97-2.05 (7H, m), 2.31 (7H, m), 3.39 (3H, br s), 4.06 (2H, t, J=6.96 Hz), 4.14 (2H, t, J=6.62 Hz), 4.26-4.35 (1H, m), 4.48-4.55 (1H, m), 4.80 (1H, m), 5.34 (3H, m), 5.49 (1H, m). MS [ESI]: m/z: [M+H] calc 762.6 obs. 762.3.

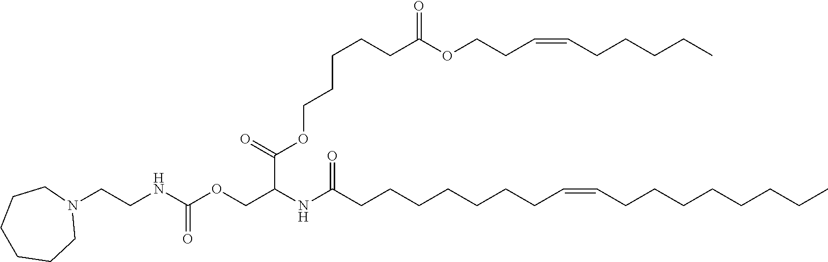 [SMILES: CCCCC/C=C\CCOC(=O)CCCCCOC(=O)C(COC(=O)NCCN1CCCCCC1)NC(=O)CCCCCCC/C=C\CCCCCCCC]

(Z)-Non-3-en-1-yl 6-((O-((2-(azepan-1-yl)ethyl)carbamoyl)-N-oleoylseryl)oxy)hexanoate was synthesized according to representative synthetic scheme 1 and the corresponding methods. ¹H NMR (400 MHz, CDCl₃): δ 0.88 (6H, m), 1.28 (30H, m), 1.64 (13H, m), 1.97-2.06 (6H, m), 2.29 (6H, m), 2.69-2.87 (6H, m), 3.26-3.36 (2H, m), 4.06 (2H, t, J=6.96 Hz), 4.14 (2H, t, J=6.67 Hz), 4.32 (1H, m), 4.48 (1H, dd, J=11.37, 3.86 Hz), 4.79 (1H, m), 5.33 (3H, m), 5.49 (1H, m), 6.60-6.75 (1H, m). MS [ESI]: m/z: [M+H] calc 776.6 obs. 776.4.

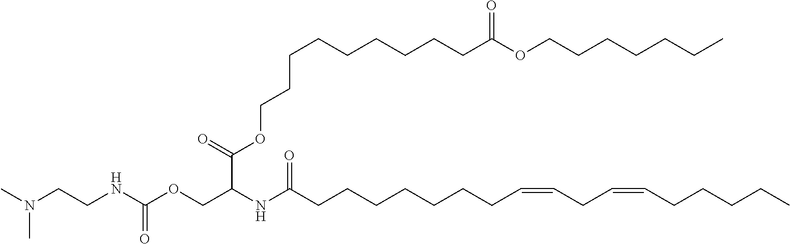 [SMILES: CCCCC/C=C\C/C=C\CCCCCCCC(=O)NC(COC(=O)NCCN(C)C)C(=O)OCCCCCCCCCC(=O)OCCCCCCC]

Heptyl 10-((O-((2-(dimethylamino)ethyl)carbamoyl)-N-((9Z,12Z)-octadeca-9,12-dienoyl)seryl)oxy)decanoate was synthesized according to representative synthetic scheme 1 and the corresponding methods. ¹H NMR (400 MHz, CDCl₃): δ 0.89 (6H, td, J=6.85, 1.97 Hz), 1.31 (33H, m), 1.62 (9H, s), 2.04 (4H, m), 2.22 (10H, s), 2.40 (2H, m), 2.77 (2H, t, J=6.05 Hz), 3.24 (1H, m), 4.05 (2H, t, J=6.75 Hz), 4.15 (2H, t, J=6.75 Hz), 4.30-4.36 (1H, m), 4.41-4.47 (1H, m), 4.80 (1H, m), 5.35 (4H, m), 6.35-6.41 (1H, m). MS [ESI]: m/z: [M+H] calc 750.6 obs. 750.5.

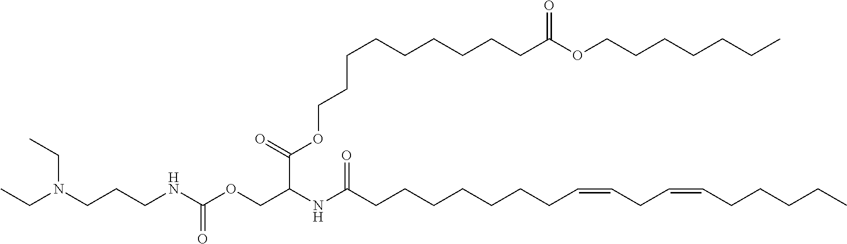 [SMILES: CCCCC/C=C\C/C=C\CCCCCCCC(=O)NC(COC(=O)NCCCN(CC)CC)C(=O)OCCCCCCCCCC(=O)OCCCCCCC]

Heptyl 10-((O-((3-(diethylamino)propyl)carbamoyl)-N-((9Z,12Z)-octadeca-9,12-dienoyl)seryl)oxy)decanoate was synthesized according to representative synthetic scheme 1 and the corresponding methods. ¹H NMR (400 MHz, CDCl₃): δ 0.89 (6H, td, J=6.89, 2.01 Hz), 1.02 (6H, t, J=7.13 Hz), 1.30 (33H, m), 1.62 (10H, m), 2.01-2.08 (4H, m), 2.26 (4H, m), 2.48 (6H, m), 2.77 (2H, t, J=6.04 Hz), 3.25 (2H, m), 4.05 (2H, t, J=6.75 Hz), 4.13 (2H, t, J=6.75 Hz), 4.30-4.36 (1H, m), 4.38-4.45 (1H, m), 4.77 (1H, m), 5.35 (4H, m), 6.40 (1H, m). MS [ESI]: m/z: [M+H] calc 792.6 obs. 792.8.

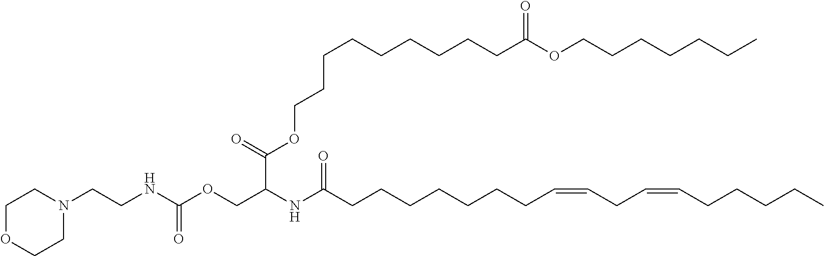 [SMILES: CCCCC/C=C\C/C=C\CCCCCCCC(=O)NC(COC(=O)NCCN1CCOCC1)C(=O)OCCCCCCCCCC(=O)OCCCCCCC]

Heptyl 10-((O-((2-morpholinoethyl)carbamoyl)-N-((9Z,12Z)-octadeca-9,12-dienoyl)seryl)oxy)decanoate was synthesized according to representative synthetic scheme 1 and the corresponding methods. ¹H NMR (400 MHz, CDCl₃): δ 0.89 (6H, td, J=6.90, 2.02 Hz), 1.30 (33H, m), 1.60 (8H, m), 2.04 (4H, m), 2.26 (4H, m), 2.44 (6H, m), 2.77 (2H, m), 3.27 (1H, m), 3.70 (4H, m), 4.05 (2H, t, J=6.75 Hz), 4.15 (2H, t, J=6.91 Hz), 4.31-4.38 (1H, m), 4.44 (1H, m), 4.81 (1H, m), 5.17-5.22 (1H, m), 5.35 (4H, m), 6.36 (1H, m). MS [ESI]: m/z: [M+H] calc 792.6 obs. 792.7.

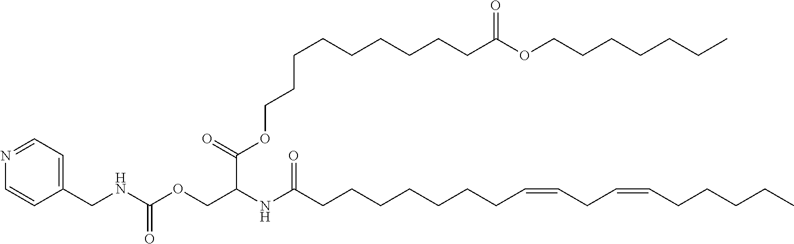 [SMILES: CCCCC/C=C\C/C=C\CCCCCCCC(=O)NC(COC(=O)NCc1ccncc1)C(=O)OCCCCCCCCCC(=O)OCCCCCCC]

Heptyl 10-((N-((9Z,12Z)-octadeca-9,12-dienoyl)-O-((pyridin-4-ylmethyl)carbamoyl)seryl)oxy)decanoate was synthesized according to representative synthetic scheme 1 and the corresponding methods. ¹H NMR (400 MHz, CDCl₃): δ 0.88 (6H, m), 1.29 (33H, m), 1.62 (7H, m), 2.00-2.07 (4H, 2.02 (s), 2.04 (d, J=6.15 Hz)), 2.27 (3H, q, J=7.67 Hz), 2.74-2.78 (2H, 2.75 (s), 2.76 (s)), 4.05 (4H, m), 4.52 (4H, m), 4.85 (1H, m), 5.34 (4H, m), 5.73-5.78 (1H, m), 6.42 (1H, d, J=8.03 Hz), 7.58 (2H, m), 8.61 (2H, d, J=5.97 Hz). MS [ESI]: m/z: [M+H] calc 770.6 obs. 770.5.

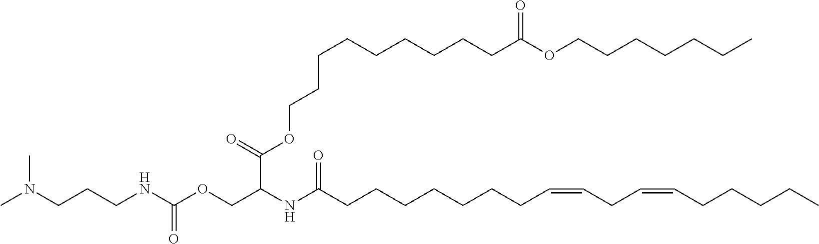 [SMILES: CCCCC/C=C\C/C=C\CCCCCCCC(=O)NC(COC(=O)NCCCN(C)C)C(=O)OCCCCCCCCCC(=O)OCCCCCCC]

Heptyl 10-((O-((3-(dimethylamino)propyl)carbamoyl)-N-((9Z,12Z)-octadeca-9,12-dienoyl)seryl)oxy)decanoate was synthesized according to representative synthetic scheme 1 and the corresponding methods. ¹H NMR (400 MHz, CDCl₃): δ 0.89 (6H, td, J=6.89, 1.99 Hz), 1.30 (33H, m), 1.63 (11H, m), 2.05 (4H, m), 2.26 (10H, m), 2.36-2.42 (2H, m), 2.75 (2H, t, J=6.76 Hz), 3.25 (1H, m), 4.05 (2H, t, J=6.75 Hz), 4.14 (2H, t, J=6.74 Hz), 4.41-4.46 (1H, m), 5.35 (3H, m), 4.30-4.37 (1H, m), 4.75-4.82 (1H, m), 5.70-5.77 (1H, m), 6.38-6.45 (1H, m). MS [ESI]: m/z: [M+H] calc 764.6 obs. 764.8.

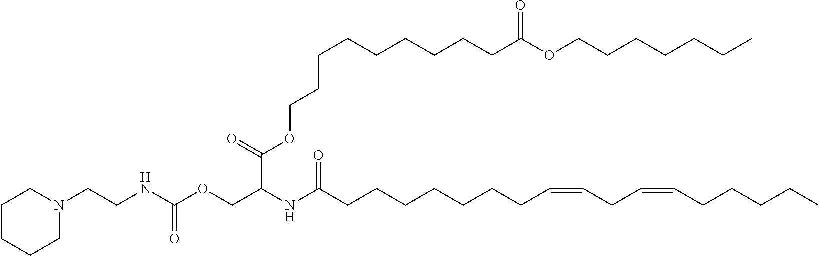 [SMILES: CCCCC/C=C\C/C=C\CCCCCCCC(=O)NC(COC(=O)NCCN1CCCCC1)C(=O)OCCCCCCCCCC(=O)OCCCCCCC]

Heptyl 10-((N-((9Z,12Z)-octadeca-9,12-dienoyl)-O-((2-(piperidin-1-yl)ethyl)carbamoyl)seryl)oxy)decanoate was synthesized according to representative synthetic scheme 1 and the corresponding methods. ¹H NMR (400 MHz, CDCl₃): δ 0.89 (6H, td, J=6.88, 1.99 Hz), 1.30 (33H, m), 1.44 (2H, m), 1.60 (18H, m), 2.05 (4H, m), 2.29 (4H, m), 2.38-2.43 (2H, m), 2.77 (2H, t, J=5.95 Hz), 3.24 (1H, m), 4.05 (2H, t, J=6.75 Hz), 4.15 (2H, t, J=6.76 Hz), 4.31-4.37 (1H, m), 4.42-4.47 (1H, m), 4.80 (1H, m), 5.35 (5H, m), 6.41 (1H, d, J=7.57 Hz). MS [ESI]: m/z: [M+H] calc 790.6 obs. 790.7.

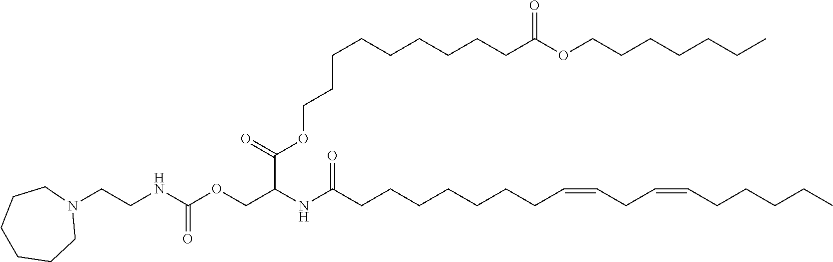 [SMILES: CCCCC/C=C\C/C=C\CCCCCCCC(=O)NC(COC(=O)NCCN1CCCCCC1)C(=O)OCCCCCCCCCC(=O)OCCCCCCC]

Heptyl 10-((O-((2-(azepan-1-yl)ethyl)carbamoyl)-N-((9Z,12Z)-octadeca-9,12-dienoyl)seryl)oxy)decanoate was synthesized according to representative synthetic scheme 1 and the corresponding methods. ¹H NMR (400 MHz, CDCl₃): δ 0.89 (6H, m), 1.30 (33H, m), 1.62 (20H, s), 2.05 (2H, m), 2.28 (4H, m), 2.75-2.79 (2H, m), 2.85-3.09 (4H, m), 3.34-3.50 (2H, m)4.05 (2H, t, J=6.75 Hz), 4.12 (2H, m), 4.23-4.30 (1H, m), 4.50-4.57 (1H, m)4.77-4.82 (1H, m), 5.35 (4H, m). MS [ESI]: m/z: [M+H] calc 804.6 obs. 804.7.

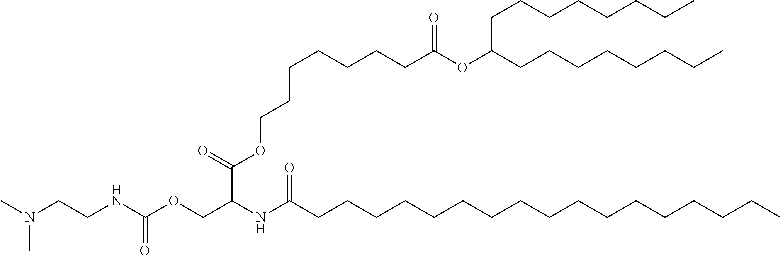 [SMILES: CCCCCCCCCCCCCCCCCC(=O)NC(COC(=O)NCCN(C)C)C(=O)OCCCCCCCC(=O)OC(CCCCCCCC)CCCCCCCC]

Heptadecan-9-yl 8-((O-((2-(dimethylamino)ethyl)carbamoyl)-N-stearoylseryl)oxy)octanoate was synthesized according to representative synthetic scheme 1 and the corresponding methods. ¹H NMR (400 MHz, CDCl₃): δ 0.84-0.91 (9H, 0.87 (t, J=6.78 Hz), 0.88 (s)), 1.25 (58H, m), 1.45-1.54 (5H, m), 1.58-1.68 (6H, m), 2.27 (2H, t, J=7.52 Hz), 2.30-2.36 (2H, m), 2.66 (5H, br s), 2.81-3.00 (2H, m), 3.45 (2H, m), 4.11 (2H, m), 4.26-4.33 (1H, m), 4.51-4.58 (1H, m), 4.76-4.90 (2H, m), 6.18-6.26 (1H, m), 7.01-7.14 (1H, m). MS [ESI]: m/z: [M+H] calc 866.8 obs. 867.0.

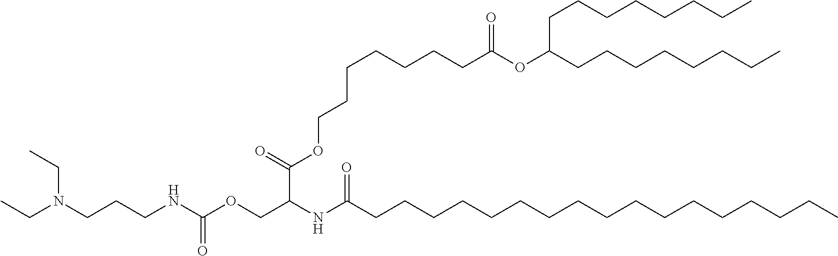 [SMILES: CCCCCCCCCCCCCCCCCC(=O)NC(COC(=O)NCCCN(CC)CC)C(=O)OCCCCCCCC(=O)OC(CCCCCCCC)CCCCCCCC]

Heptadecan-9-yl 8-((O-((3-diethylamino)propyl)carbamoyl)-N-stearoylseryl)oxy)octanoate was synthesized according to representative synthetic scheme 1 and the corresponding methods. ¹H NMR (400 MHz, CDCl₃): δ 0.88 (9H, t, J=6.71 Hz), 1.25 (58H, m), 1.39 (6H, m), 1.60-1.67 (7H, m), 1.46-1.54 (4H, m), 2.04 (2H, m), 2.25-2.31 (4H, 2.28 (t, J=7.48 Hz), 2.28 (t, J=7.84 Hz)), 2.99-3.24 (6H, m), 3.34 (1H, q, J=6.05 Hz), 4.13 (2H, m), 4.23-4.31 (1H, m), 4.45-4.51 (1H, m), 4.76-4.89 (2H, m), 5.95-6.02 (1H, m), 6.57-6.65 (1H, m). MS [ESI]: m/z: [M+H] calc 908.8 obs. 908.9.

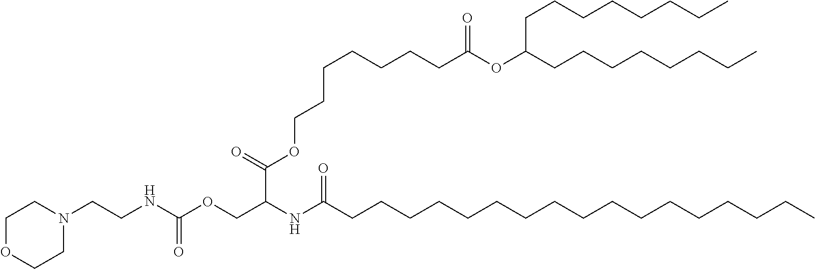 [SMILES: CCCCCCCCCCCCCCCCCC(=O)NC(COC(=O)NCCN1CCOCC1)C(=O)OCCCCCCCC(=O)OC(CCCCCCCC)CCCCCCCC]

Heptadecan-9-yl 8-((O-((2-morpholinoethyl)carbamoyl)-N-stearoylseryl)oxy)octanoate was synthesized according to representative synthetic scheme 1 and the corresponding methods. ¹H NMR (400 MHz, CDCl₃): δ 0.88 (9H, t, J=6.80 Hz), 1.26 (57H, m), 1.46-1.54 (4H, m), 1.60 (11H, m), 2.27 (2H, t, J=7.50 Hz), 2.35-2.42 (2H, m), 2.80-2.95 (2H, m), 3.06-3.28 (3H, m), 3.48-3.74 (511, m), 3.94-4.02 (2H, m), 4.13 (3H, m), 4.21-4.29 (1H, m), 4.32-4.42 (2H, m), 4.55-4.62 (1H, m), 4.78-4.89 (2H, m), 6.81-6.87 (1H, m), 7.30-7.37 (1H, m). MS [ESI]: m/z: [M+H] calc 908.8 obs. 908.9.

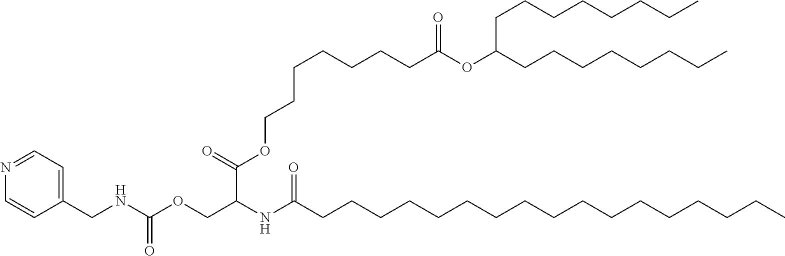 [SMILES: CCCCCCCCCCCCCCCCCC(=O)NC(COC(=O)NCc1ccncc1)C(=O)OCCCCCCCC(=O)OC(CCCCCCCC)CCCCCCCC]

Heptadecan-9-yl 8-((O-((pyridin-4-ylmethyl)carbamoyl)-N-stearoylseryl)oxy)octanoate was synthesized according to representative synthetic scheme 1 and the corresponding methods. ¹H NMR (400 MHz, CDCl₃): δ 0.88 (9H, t, J=6.68 Hz), 1.25 (54H, s), 1.45-1.54 (6H, m), 1.57-1.68 (11H, m)2.26 (3H, q, J=7.50 Hz), 4.14 (OH, m), 4.45 (2H, m), 4.63 (2H, m), 4.84 (1H, m), 6.11-6.18 (1H, m), 6.50 (1H, d, J=8.20 Hz), 7.89 (2H, d, J=5.47 Hz), 8.65 (2H, m). MS [ESI]: m/z: [M+H] calc 886.7 obs. 886.8.

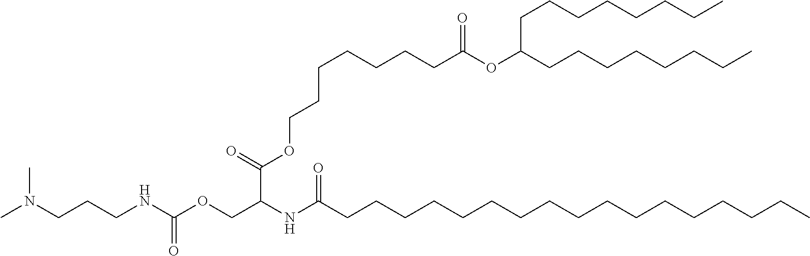 [SMILES: CCCCCCCCCCCCCCCCCC(=O)NC(COC(=O)NCCCN(C)C)C(=O)OCCCCCCCC(=O)OC(CCCCCCCC)CCCCCCCC]

Heptadecan-9-yl 8-((O-((3-(dimethylamino)propyl)carbamoyl)-N-stearoylseryl)oxy)octanoate was synthesized according to representative synthetic scheme 1 and the corresponding methods. ¹H NMR (400 MHz, CDCl₃): δ 0.87 (9H, t, J=6.66 Hz), 1.25 (58H, m), 2.27 (2H, m), 2.60 (5H, s), 1.45-1.54 (4H, m), 1.56-1.68 (6H, m), 1.87-1.95 (2H, m), 2.79-2.86 (2H, m), 3.31 (2H, q, J=5.99 Hz), 4.13 (2H, td, J=6.75, 1.84 Hz), 4.30 (1H, m), 4.45 (1H, dd, J=11.34, 4.06 Hz), 4.82 (2H, m), 5.91 (1H, m), 6.59 (1H, m). MS [ESI]: m/z: [M+H] calc 880.8 obs. 880.9.

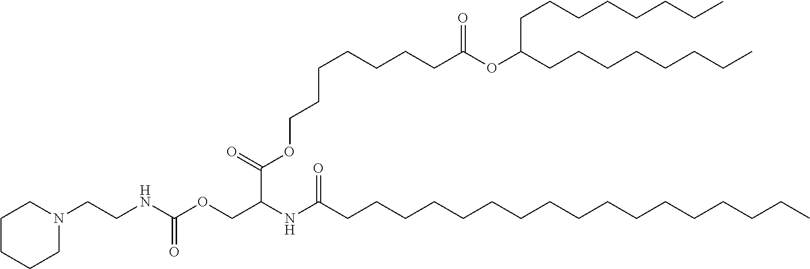 [SMILES: CCCCCCCCCCCCCCCCCC(=O)NC(COC(=O)NCCN1CCCCC1)C(=O)OCCCCCCCC(=O)OC(CCCCCCCC)CCCCCCCC]

Heptadecan-9-yl 8-((O-((2-(piperidin-1-yl)ethyl)carbamoyl)-N-stearoylseryl)oxy)octanoate was synthesized according to representative synthetic scheme 1 and the corresponding methods. ¹H NMR (400 MHz, CDCl₃): δ 0.88 (9H, t, J=6.80 Hz), 1.25 (56H, m), 1.45-1.54 (5H, m), 1.62 (8H, s), 1.81-1.96 (3H, m), 2.27 (2H, t, J=7.49 Hz), 2.37 (3H, t, J=7.41 Hz), 2.57-2.72 (2H, m), 2.98-3.20 (2H, m), 3.54-3.75 (4H, m), 4.11 (3H, m), 4.22-4.28 (1H, m), 4.56-4.62 (1H, m), 4.78-4.89 (2H, m), 6.97-7.04 (1H, m), 7.19-7.24 (1H, m). MS [ESI]: m/z: [M+H] calc 906.8 obs. 906.9.

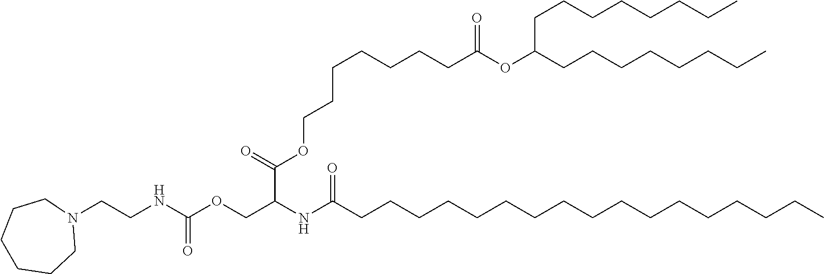 [SMILES: CCCCCCCCCCCCCCCCCC(=O)NC(COC(=O)NCCN1CCCCCC1)C(=O)OCCCCCCCC(=O)OC(CCCCCCCC)CCCCCCCC]

Heptadecan-9-yl 8-((O-((2-(azepan-1-yl)ethyl)carbamoyl)-N-stearoylseryl)oxy)octanoate was synthesized according to representative synthetic scheme 1 and the corresponding methods. ¹H NMR (400 MHz, CDCl₃): δ 0.89 (9H, t, J=6.81 Hz), 1.27 (59H, m), 1.52 (4H, m), 1.61-1.75 (15H, m), 2.28 (2H, t, J=7.52 Hz), 2.35 (2H, t, J=7.57 Hz), 1.84-1.96 (3H, m), 2.94-3.13 (3H, m), 3.43-3.53 (2H, m), 4.14 (2H, m), 4.24-4.32 (1H, m), 4.53-4.60 (1H, m), 4.79-4.91 (2H, m). MS [ESI]: m/z: [M+H] calc 920.8 obs. 920.9.

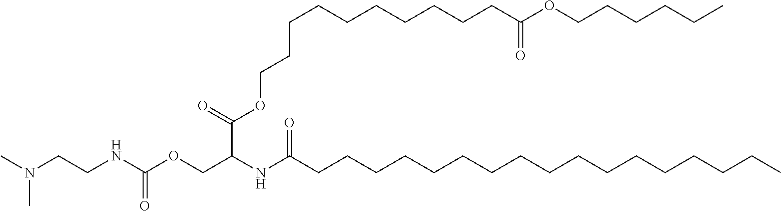 [SMILES: CCCCCCCCCCCCCCCCCC(=O)NC(COC(=O)NCCN(C)C)C(=O)OCCCCCCCCCCC(=O)OCCCCCC]

Hexyl 11-((O-((2-(dimethylamino)ethyl)carbamoyl)-N-stearoylseryl)oxy)undecanoate was synthesized according to representative synthetic scheme 1 and the corresponding methods. ¹H NMR (500 MHz, CDCl₃): δ 0.86 (6H, m), 1.23 (46H, m), 1.59 (8H, m), 2.26 (2H, t, J=7.54 Hz), 2.30-2.35 (2H, m), 3.44-3.50 (2H, m), 3.69 (1H, q, J=7.00 Hz), 2.73 (5H, br s), 4.03 (3H, t, J=6.74 Hz), 4.06-4.14 (3H, m), 4.25-4.30 (1H, m), 4.49-4.55 (1H, m), 4.76-4.82 (2H, m). MS [ESI]: m/z: [M+H] calc 754.6 obs. 754.7.

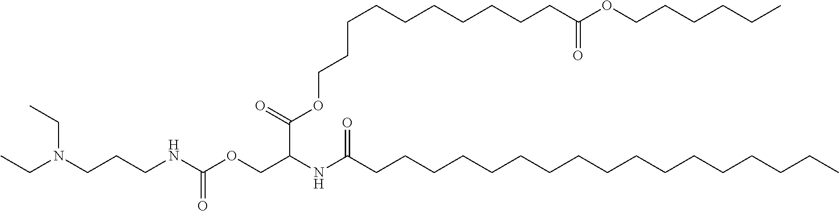 [SMILES: CCCCCCCCCCCCCCCCCC(=O)NC(COC(=O)NCCCN(CC)CC)C(=O)OCCCCCCCCCCC(=O)OCCCCCC]

Hexyl 11-((O-((3-(diethylamino)propyl)carbamoyl)-N-stearoylseryl)oxy)undecanoate was synthesized according to representative synthetic scheme 1 and the corresponding methods. ¹H NMR (500 MHz, CDCl₃): δ 0.86 (6H, d, J=5.88 Hz), 1.23 (51H, m), 1.54-1.63 (9H, m), 1.85-1.94 (2H, m), 2.26 (4H, m), 2.79-2.95 (6H, m), 3.25-3.33 (2H, m), 4.03 (2H, t, J=6.68 Hz), 4.10 (2H, m), 4.26-4.29 (1H, m), 4.42-4.45 (1H, m), 4.74-4.79 (1H, m), 6.05-6.12 (1H, m), 6.50-6.54 (1H, m). MS [ESI]: m/z: [M+H] calc 796.7 obs. 796.8.

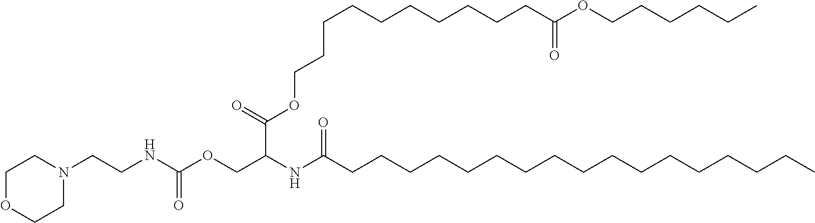 [SMILES: CCCCCCCCCCCCCCCCCC(=O)NC(COC(=O)NCCN1CCOCC1)C(=O)OCCCCCCCCCCC(=O)OCCCCCC]

Hexyl 11-((O-((2-morpholinoethyl)carbamoyl)-N-stearoylseryl)oxy)undecanoate was synthesized according to representative synthetic scheme 1 and the corresponding methods. ¹H NMR (500 MHz, CDCl₃): δ 0.86 (6H, m), 1.23 (48H, s), 1.60 (15H, m), 2.26 (2H, t, J=7.27 MHz), 2.29-2.34 (2H, m), 3.45-3.52 (2H, m), 4.03 (6H, m), 4.25-4.29 (1H, m), 4.49-4.55 (1H, m), 4.78-4.82 (1H, m). MS [ESI]: m/z: [M+H] calc 796.6 obs. 796.8.

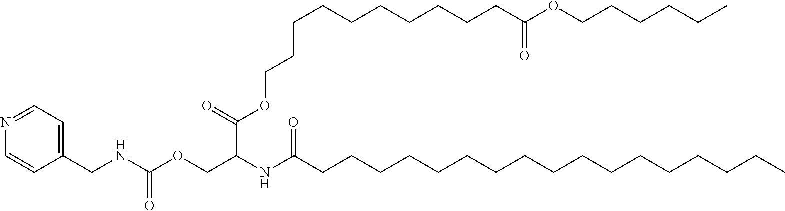 [SMILES: CCCCCCCCCCCCCCCCCC(=O)NC(COC(=O)NCc1ccncc1)C(=O)OCCCCCCCCCCC(=O)OCCCCCC]

Hexyl 11-((O-((pyridin-4-ylmethyl)carbamoyl)-N-stearoylseryl)oxy)undecanoate was synthesized according to representative synthetic scheme 1 and the corresponding methods. ¹H NMR (500 MHz, CDCl₃): δ 0.85 (6H, m), 1.22 (46H, m), 1.55-1.65 (6H, m), 2.26 (4H, m), 4.03 (2H, t, J=6.76 Hz), 4.06-4.17 (3H, m), 4.38-4.47 (3H, m), 4.52-4.59 (2H, m), 4.81-4.85 (1H, m), 5.89-5.96 (1H, m), 6.41-6.48 (1H, m), 7.63-7.71 (2H, m), 8.60 (2H, dd, J=5.34, 2.03 Hz). MS [ESI]: m/z: [M+H] calc 774.6 obs. 774.5.

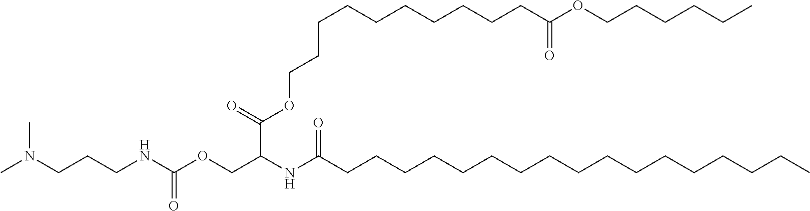 [SMILES: CCCCCCCCCCCCCCCCCC(=O)NC(COC(=O)NCCCN(C)C)C(=O)OCCCCCCCCCCC(=O)OCCCCCC]

Hexyl 11-((O-((3-(dimethylamino)propyl)carbamoyl)-N-stearoylseryl)oxy)undecanoate was synthesized according to representative synthetic scheme 1 and the corresponding methods. ¹H NMR (500 MHz, CDCl₃): δ 0.86 (6H, m), 1.23 (45H, s), 1.56-1.64 (6H, m), 1.91-1.98 (2H, m), 2.22-2.29 (4H, m), 2.65 (5H, s), 2.86-2.93 (2H, m), 3.28-3.34 (2H, m), 3.66-3.73 (2H, m), 4.03 (3H, s), 4.08-4.15 (3H, m), 4.26-4.30 (1H, m), 4.41-4.45 (1H, m), 4.77-4.81 (1H, m), 5.88-5.92 (1H, m), 6.54-6.58 (1H, m). MS [ESI]: m/z: [M+H] calc 768.6 obs. 768.7.

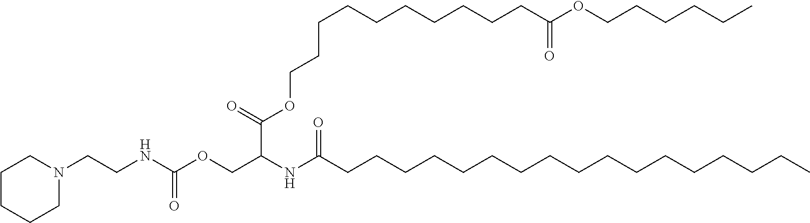 [SMILES: CCCCCCCCCCCCCCCCCC(=O)NC(COC(=O)NCCN1CCCCC1)C(=O)OCCCCCCCCCCC(=O)OCCCCCC]

Hexyl 11-((O-((2-(piperidin-1-yl)ethyl)carbamoyl)-N-stearoylseryl)oxy)undecanoate was synthesized according to representative synthetic scheme 1 and the corresponding methods. ¹H NMR (500 MHz, CDCl₃): δ 0.86 (6H, m), 1.23 (46H, m), 1.56-1.67 (12H, m), 2.26 (3H, t, J=7.46 Hz), 2.29-2.35 (2H, m), 2.88-3.02 (3H, m), 3.47-3.53 (2H, m), 4.03 (3H, t, J=6.77 Hz), 4.07-4.14 (3H, m), 4.21-4.27 (1H, m), 4.51-4.55 (1H, m), 4.77-4.83 (2H, m), 6.95-7.09 (1H, m). MS [ESI]: m/z: [M+H] calc 794.7 obs. 794.9.

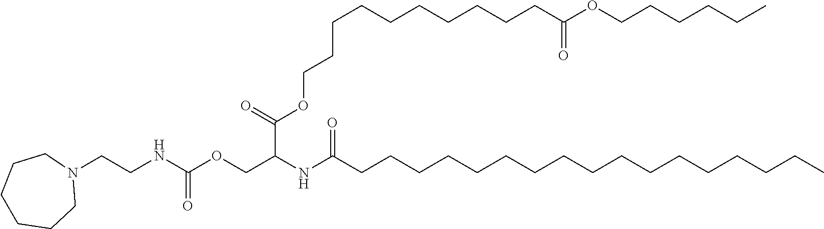 [SMILES: CCCCCCCCCCCCCCCCCC(=O)NC(COC(=O)NCCN1CCCCCC1)C(=O)OCCCCCCCCCCC(=O)OCCCCCC]

Hexyl 11-((O-((2-(azepan-1-yl)ethyl)carbamoyl)-N-stearoylseryl)oxy)undecanoate was synthesized according to representative synthetic scheme 1 and the corresponding methods. ¹H NMR (500 MHz, CDCl₃): δ 0.86-0.91 (6H, 0.88 m), 1.25 (47H, m), 1.63 (7H, m), 1.80-1.88 (4H, m), 2.28 (2H, t, J=7.53 Hz), 2.37 (2H, t, J=7.60 Hz), 2.93-3.02 (3H, m), 3.07-3.16 (2H, m), 3.16-3.23 (1H, m), 3.52-3.58 (2H, m), 3.63-3.73 (4H, m), 4.05 (3H, t, J=6.71 Hz), 4.09-4.15 (2H, m), 4.22-4.27 (1H, m), 4.56-4.60 (1H, m), 4.79-4.84 (2H, m), 6.93-6.98 (1H, m). MS [ESI]: m/z: [M+H] calc 808.7 obs. 808.8.

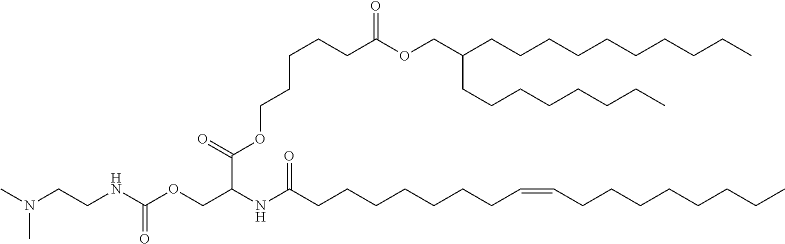 [SMILES: CCCCCCCC/C=C\CCCCCCCC(=O)NC(COC(=O)NCCN(C)C)C(=O)OCCCCCC(=O)OCC(CCCCCCCC)CCCCCCCCCC]

2-Octyldodecyl 6-((O-((2-(dimethylamino)ethyl)carbamoyl)-N-oleoylseryl)oxy)hexanoate was synthesized according to representative synthetic scheme 1 and the corresponding methods. ¹H NMR (500 MHz, CDCl₃): δ 0.88 (9H, t, J=6.92 Hz), 1.26 (53H, m), 1.60-1.70 (8H, m), 1.98-2.03 (4H, m), 2.22 (7H, m), 2.31 (2H, m), 1.35-1.43 (2H, m), 2.37-2.42 (2H, m), 3.22-3.25 (2H, m), 3.96 (2H, d, J=5.81 Hz), 4.13-4.18 (2H, m), 4.31-4.36 (1H, m), 4.40-4.45 (1H, m), 4.76-4.81 (1H, m), 5.34 (2H, m), 6.41-6.45 (1H, m). MS [ESI]: m/z: [M+H] calc 878.8 obs. 878.9.

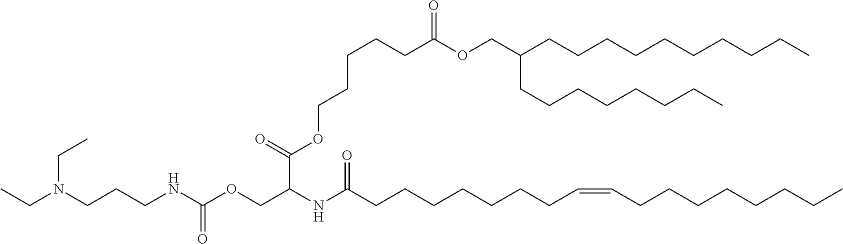 [SMILES: CCCCCCCC/C=C\CCCCCCCC(=O)NC(COC(=O)NCCCN(CC)CC)C(=O)OCCCCCC(=O)OCC(CCCCCCCC)CCCCCCCCCC]

2-Octyldodecyl 6-((O-((3-(diethylamino)propyl)carbamoyl)-N-oleoylseryl)oxy)hexanoate was synthesized according to representative synthetic scheme 1 and the corresponding methods. ¹H NMR (500 MHz, CDCl₃): δ 0.86 (9H, t, J=6.95 Hz), 0.99 (6H, t, J=7.14 Hz), 1.24 (53H, m), 1.57-1.67 (18H, m), 1.96-2.00 (4H, m), 2.20 (2H, m), 2.29 (2H, t, J=7.59 Hz), 2.46 (6H, m), 1.33-1.39 (3H, m), 3.22 (1H, m), 3.94 (2H, d, J=5.82 Hz), 4.09-4.15 (2H, m), 5.30-5.33 (2H, m), 4.29-4.34 (1H, m), 4.36-4.40 (1H, m), 4.71-4.76 (1H, m), 6.38-6.42 (1H, m), 6.43-6.46 (1H, m). MS [ESI]: m/z: [M+H] calc 920.8 obs. 920.7.

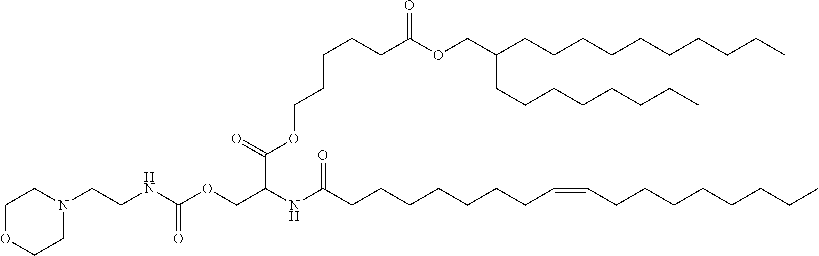 [SMILES: CCCCCCCC/C=C\CCCCCCCC(=O)NC(COC(=O)NCCN1CCOCC1)C(=O)OCCCCCC(=O)OCC(CCCCCCCC)CCCCCCCCCC]

2-Octyldodecyl 6-((O-((2-morpholinoethyl)carbamoyl)-N-oleoylseryl)oxy)hexanoate was synthesized according to representative synthetic scheme 1 and the corresponding methods. ¹H NMR (500 MHz, CDCl₃): δ 0.86 (9H, t, J=6.94 Hz), 1.24 (55H, m), 1.59-1.69 (7H, m), 1.95-2.02 (4H, m), 2.21 (1H, m), 2.29 (2H, t, J=7.48 Hz), 2.39-2.46 (6H, m), 3.68 (3H, m), 3.94 (2H, d, J=5.82 Hz), 4.11-4.16 (3H, m), 3.22-3.28 (2H, m), 4.32-4.36 (1H, m), 4.40-4.44 (1H, m), 4.76-4.80 (1H, m), 5.30-5.34 (2H, m), 6.38-6.41 (1H, m). MS [ESI]: m/z: [M+H] calc 920.8 obs. 920.6.

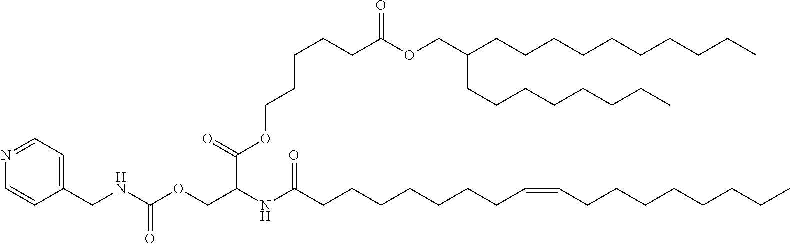 [SMILES: CCCCCCCC/C=C\CCCCCCCC(=O)NC(COC(=O)NCc1ccncc1)C(=O)OCCCCCC(=O)OCC(CCCCCCCC)CCCCCCCCCC]

2-Octyldodecyl 6-((N-oleoyl-O-((pyridin-4-ylmethyl)carbamoyl)seryl)oxy)hexanoate was synthesized according to representative synthetic scheme 1 and the corresponding methods. ¹H NMR (500 MHz, CDCl₃): δ 0.88 (9H, t, J=6.68 Hz), 1.26 (54H, m), 1.64 (7H, m), 2.00 (4H, q, J=6.49 Hz), 2.24 (2H, t, J=8.12 Hz), 2.31 (2H, t, J=8.01 Hz), 3.93 (2H, d, J=5.34 Hz), 4.13 (1H, m), 4.22 (1H, m), 4.44 (2H, m), 4.55 (2H, m), 4.84 (1H, m), 5.33 (2H, m), 6.16 (1H, m), 6.48 (1H, d, J=8.06 Hz), 7.69 (2H, d, J=5.02 Hz), 8.64 (2H, d, J=5.56 Hz). MS [ESI]: m/z: [M+H] calc 898.7 obs. 898.7.

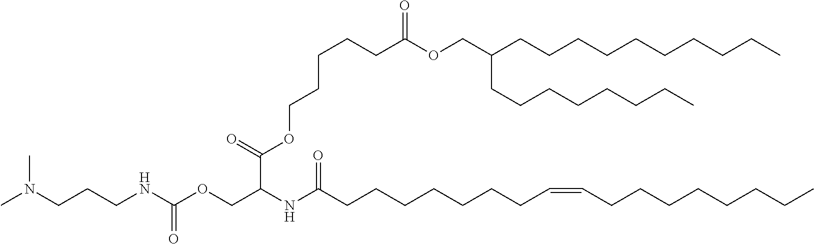 [SMILES: CCCCCCCC/C=C\CCCCCCCC(=O)NC(COC(=O)NCCCN(C)C)C(=O)OCCCCCC(=O)OCC(CCCCCCCC)CCCCCCCCCC]

2-Octyldodecyl 6-((O-((3-(dimethylamino)propyl)carbamoyl)-N-oleoylseryl)oxy)hexanoate was synthesized according to representative synthetic scheme 1 and the corresponding methods. ¹H NMR (500 MHz, CDCl₃): δ 0.88 (9H, t, J=6.94 Hz), 1.26 (52H, m), 1.60-1.70 (10H, m), 2.01 (2H, m), 2.25 (7H, s), 2.31 (2H, t, J=7.40 Hz), 2.36-2.40 (2H, m), 1.35-1.42 (3H, m), 3.21-3.27 (2H, m), 3.96 (2H, d, J=5.88 Hz), 4.12-4.19 (3H, m), 4.32-4.36 (1H, m), 4.40-4.44 (1H, m), 4.75-4.78 (1H, m), 5.32-5.36 (2H, m), 5.79-5.83 (1H, m), 6.46-6.49 (1H, m). MS [ESI]: m/z: [M+H] calc 892.8 obs. 892.6.

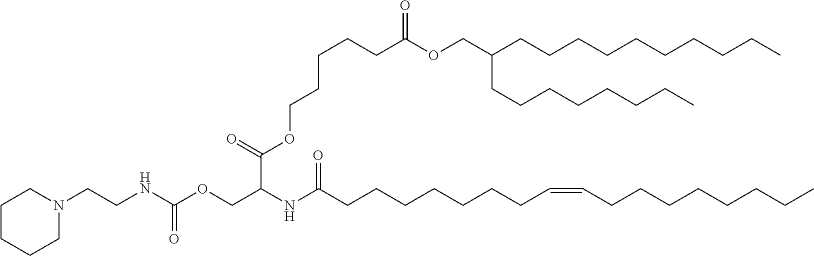 [SMILES: CCCCCCCC/C=C\CCCCCCCC(=O)NC(COC(=O)NCCN1CCCCC1)C(=O)OCCCCCC(=O)OCC(CCCCCCCC)CCCCCCCCCC]

2-Octyldodecyl 6-((N-oleoyl-O-((2-(piperidin-1-yl)ethyl)carbamoyl)seryl)oxy)hexanoate was synthesized according to representative synthetic scheme 1 and the corresponding methods. ¹H NMR (500 MHz, CDCl₃): δ 0.88 (9H, t, J=6.93 Hz), 1.26 (59H, m), 1.53-1.58 (4H, m), 1.60-1.71 (5H, m), 1.98-2.03 (4H, m), 2.22-2.26 (2H, m), 2.31 (3H, t, J=7.48 Hz), 2.34-2.42 (5H, m), 3.21-3.27 (2H, m), 3.96 (2H, d, J=5.77 Hz), 4.14-4.19 (2H, m), 4.31-4.37 (1H, m), 4.41-4.46 (1H, m), 4.76-4.81 (1H, m), 5.32-5.36 (2H, m), 6.43-6.47 (1H, m). MS [ESI]: m/z: [M+H] calc 918.8 obs. 918.5.

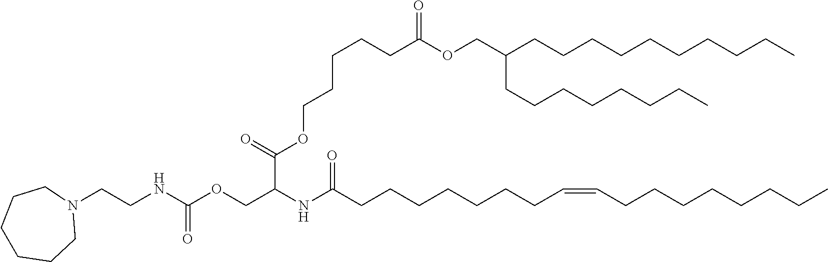 [SMILES: CCCCCCCC/C=C\CCCCCCCC(=O)NC(COC(=O)NCCN1CCCCCC1)C(=O)OCCCCCC(=O)OCC(CCCCCCCC)CCCCCCCCCC]

2-Octyldodecyl 6-((O-((2-(azepan-1-yl)ethyl)carbamoyl)-N-oleoylseryl)oxy)hexanoate was synthesized according to representative synthetic scheme 1 and the corresponding methods. ¹H NMR (500 MHz, CDCl₃): δ 0.88 (9H, t, J=6.94 Hz), 1.26 (60H, m), 1.61 (13H, m), 1.98-2.03 (4H, m), 2.24 (2H, m), 2.31 (2H, t, J=7.40 Hz), 2.57-2.63 (5H, m), 3.96 (2H, d, J=5.88 Hz), 4.16 (2H, td, J=6.59, 1.73 Hz), 4.32-4.37 (1H, m), 4.42-4.45 (1H, m), 4.77-4.81 (1H, m), 5.32-5.36 (2H, m), 6.42-6.48 (1H, m). MS [ESI]: m/z: [M+H] calc 932.8 obs. 932.6.

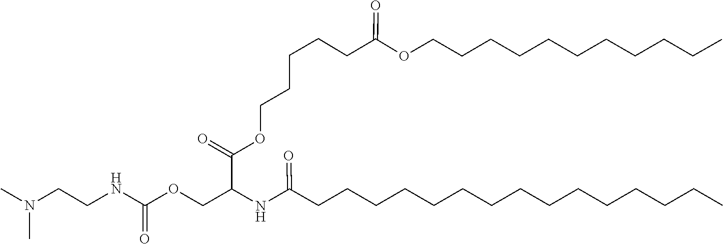 [SMILES: CCCCCCCCCCCCCCCC(=O)NC(COC(=O)NCCN(C)C)C(=O)OCCCCCC(=O)OCCCCCCCCCCC]

Undecyl 6-((O-((2-(dimethylamino)ethyl)carbamoyl)-N-palmitoylseryl)oxy)hexanoate was synthesized according to representative synthetic scheme 1 and the corresponding methods. ¹H NMR (400 MHz, CDCl₃): δ 0.88 (6H, t, J=6.81 Hz), 1.25 (40H, m), 1.36-1.42 (2H, m), 1.64 (8H, m), 2.31 (4H, t, J=7.46 Hz), 2.58 (5H, s), 2.73-2.90 (2H, m), 3.36-3.45 (2H, m), 4.05 (2H, t, J=6.78 Hz), 4.13 (2H, t, J=6.58 Hz), 4.32 (1H, dd, J=11.31, 3.10 Hz), 4.50 (1H, dd, J=11.31, 3.37 Hz), 4.80 (1H, dt, J=7.98, 3.43 Hz), 6.00-6.11 (1H, m) 7.00 (1H, br s). MS [ESI]: m/z: [M+H] calc 726.6 obs. 726.3.

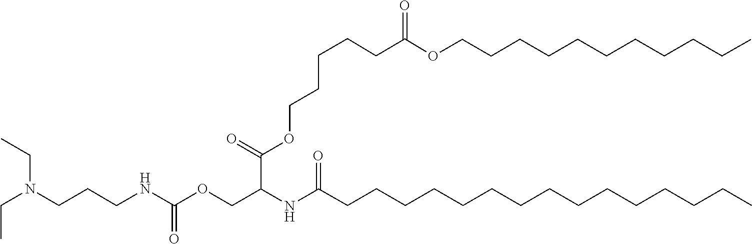 [SMILES: CCCCCCCCCCCCCCCC(=O)NC(COC(=O)NCCCN(CC)CC)C(=O)OCCCCCC(=O)OCCCCCCCCCCC]

Undecyl 6-((O-((3-(diethylamino)propyl)carbamoyl)-N-palmitoylseryl)oxy)hexanoate was synthesized according to representative synthetic scheme 1 and the corresponding methods. ¹H NMR (400 MHz, CDCl₃): δ 0.88 (6H, t, J=6.82 Hz), 1.26 (49H, m), 1.64 (8H, m), 2.29 (4H, m), 1.89-2.00 (2H, m), 2.84-3.02 (5H, m), 3.30 (2H, dd, J=6.58, 5.89 Hz), 4.05 (2H, t, J=6.78 Hz), 4.15 (2H, t, J=6.75 Hz), 4.32 (1H, m), 4.46 (1H, dd, J=11.37, 3.99 Hz), 4.79 (1H, m), 6.14-6.20 (1H, m), 6.62 (1H, d, J=7.78 Hz). MS [ESI]: m/z: [M+H] calc 768.6 obs. 768.4.

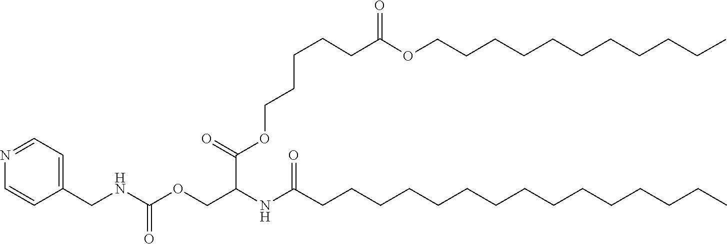 [SMILES: CCCCCCCCCCCCCCCC(=O)NC(COC(=O)NCc1ccncc1)C(=O)OCCCCCC(=O)OCCCCCCCCCCC]

Undecyl 6-((N-palmitoyl-O-((pyridin-4-ylmethyl)carbamoyl)seryl)oxy)hexanoate was synthesized according to representative synthetic scheme 1 and the corresponding methods. ¹H NMR (400 MHz, CDCl₃): δ 0.88 (6H, s), 1.25 (40H, m), 1.35-1.43 (2H, m), 1.57-1.70 (7H, m), 2.27 (4H, m), 4.02 (2H, t, J=6.73 Hz), 4.13 (2H, m), 4.17-4.24 (1H, m), 4.44 (2H, d, J=3.67 Hz), 4.53 (2H, d, J=5.35 Hz), 4.84 (1H, m), 6.15-6.21 (1H, m), 6.51 (1H, d, J=7.34 Hz), 7.64 (2H, d, J=6.85 Hz), 8.63 (2H, dd, J=4.69, 1.44 Hz). MS [ESI]: m/z: [M+H] calc 746.6 obs. 746.5.

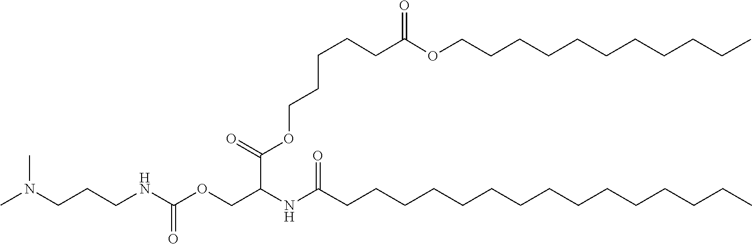 [SMILES: CCCCCCCCCCCCCCCC(=O)NC(COC(=O)NCCCN(C)C)C(=O)OCCCCCC(=O)OCCCCCCCCCCC]

Undecyl 6-((O-((3-(dimethylamino)propyl)carbamoyl)-N-palmitoylseryl)oxy)hexanoate was synthesized according to representative synthetic scheme 1 and the corresponding methods. ¹H NMR (400 MHz, CDCl₃): δ 0.88 (6H, t, J=6.79 Hz), 1.26 (44H, m), 1.64 (7H, m), 2.28 (4H, m), 2.54 (5H, s), 1.83-1.92 (2H, m), 2.70-2.77 (2H, m), 3.25-3.35 (2H, m), 4.05 (2H, t, J=6.78 Hz), 4.16 (2H, td, J=6.54, 2.83 Hz), 4.29-4.35 (1H, 4.31 (d, J=3.56 Hz), 4.34 (d, J=2.93 Hz)), 4.45 (1H, dd, J=11.35, 3.99 Hz), 4.80 (1H, m), 5.94 (1H, m), 6.59 (1H, m). MS [ESI]: m/z: [M+H] calc 740.6 obs. 740.4.

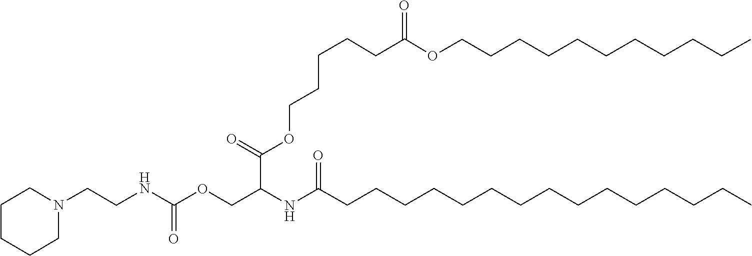 [SMILES: CCCCCCCCCCCCCCCC(=O)NC(COC(=O)NCCN1CCCCC1)C(=O)OCCCCCC(=O)OCCCCCCCCCCC]

Undecyl 6-((N-palmitoyl-O-((2-(piperidin-1-yl)ethyl)carbamoyl)seryl)oxy)hexanoate was synthesized according to representative synthetic scheme 1 and the corresponding methods. ¹H NMR (400 MHz, CDCl₃): δ 0.88 (6H, t, J=6.80 Hz), 1.25 (45H, m), 1.56-1.75 (11H, m), 1.82-1.97 (3H, m), 2.31 (4H, m), 2.71-2.97 (4H, m), 3.40-3.50 (2H, m), 4.05 (2H, t, J=6.78 Hz), 4.13 (2H, t, J=6.59 Hz), 4.27 (1H, m), 4.51-4.57 (1H, m), 4.80 (1H, m). MS [ESI]: m/z: [M+H] calc 766.6 obs. 766.3.

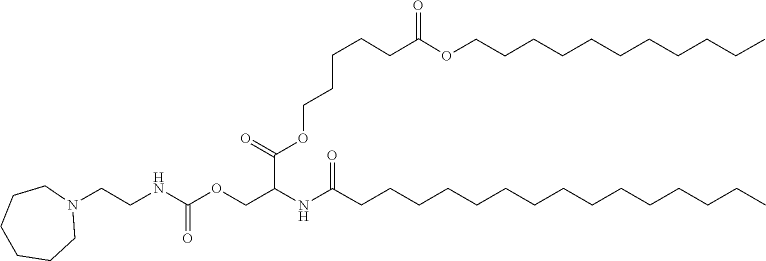 [SMILES: CCCCCCCCCCCCCCCC(=O)NC(COC(=O)NCCN1CCCCCC1)C(=O)OCCCCCC(=O)OCCCCCCCCCCC]

Undecyl 6-((O-((2-(azepan-1-yl)ethyl)carbamoyl)-N-palmitoylseryl)oxy)hexanoate was synthesized according to representative synthetic scheme 1 and the corresponding methods. ¹H NMR (400 MHz, CDCl₃): δ 0.90 (6H, t, J=6.83 Hz), 1.27 (44H, m), 1.58-1.73 (13H, m), 2.33 (4H, m), 1.83-1.99 (4H, m), 2.88-3.20 (5H, m), 3.41-3.54 (2H, m), 4.07 (2H, t, J=6.75 Hz), 4.15 (2H, t, J=6.55 Hz), 4.25-4.33 (1H, m), 4.53-4.61 (1H, m), 4.79-4.85 (1H, m). MS [ESI]: m/z: [M+H] calc 780.6 obs. 780.4.

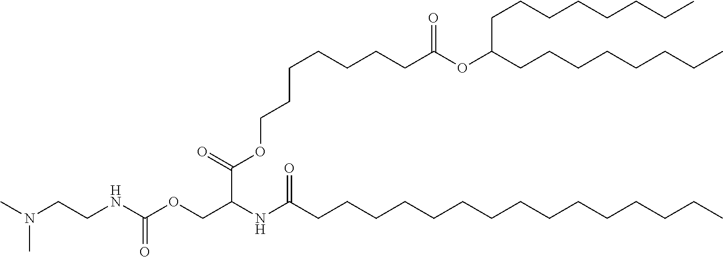 [SMILES: CCCCCCCCCCCCCCCC(=O)NC(COC(=O)NCCN(C)C)C(=O)OCCCCCCCC(=O)OC(CCCCCCCC)CCCCCCCC]

heptadecan-9-yl 8-((O-((2-(dimethylamino)ethyl)carbamoyl)-N-palmitoylseryl)oxy)octanoate was synthesized according to representative synthetic scheme 1 and the corresponding methods. ¹H NMR (400 MHz, CDCl₃): δ 0.87 (9H, t, J=6.76 Hz), 1.25 (54H, m), 1.45-1.56 (5H, m), 1.58-1.68 (7H, m), 2.27 (2H, t, J=7.48 Hz), 2.35 (2H, t, J=7.34 Hz), 2.77 (6H, s), 3.50 (1H, m), 4.10 (2H, m), 4.27-4.32 (1H, m), 4.54 (1H, m), 4.84 (2H, m), 2.90-3.13 (2H, m), 6.41-6.49 (1H, m). MS [ESI]: m/z: [M+H] calc. 837.9 obs. 839.7.

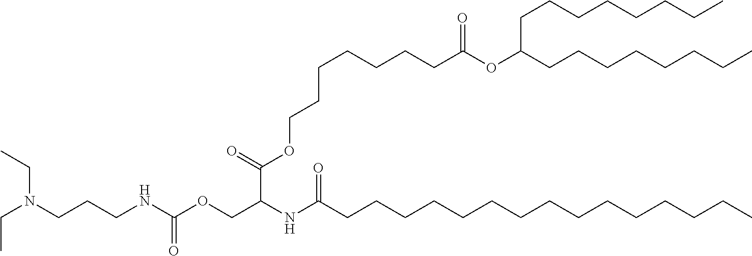 [SMILES: CCCCCCCCCCCCCCCC(=O)NC(COC(=O)NCCCN(CC)CC)C(=O)OCCCCCCCC(=O)OC(CCCCCCCC)CCCCCCCC]

heptadecan-9-yl 8-((O-((3-(diethylamino)propyl)carbamoyl)-N-palmitoylseryl)oxy)octanoate was synthesized according to representative synthetic scheme 1 and the corresponding methods. ¹H NMR (400 MHz, CDCl₃): δ 0.88 (9H, m), 1.26 (61H, m), 1.47-1.54 (4H, m), 1.63 (5H, m), 2.27 (4H, q, J=7.34 Hz), 1.84-1.93 (2H, m), 2.76-2.91 (5H, m), 3.26-3.35 (2H, m), 4.13 (2H, td, J=6.74, 1.96 Hz), 4.32 (1H, m), 4.46 (1H, dd, J=11.35, 4.10 Hz), 4.83 (2H, m), 6.14-6.18 (1H, m), 6.54 (1H, m). MS [ESI]: m/z: [M+H] calc. 880.7 obs. 881.0.

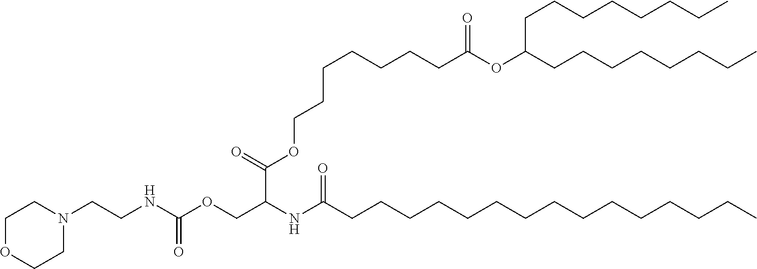 [SMILES: CCCCCCCCCCCCCCCC(=O)NC(COC(=O)NCCN1CCOCC1)C(=O)OCCCCCCCC(=O)OC(CCCCCCCC)CCCCCCCC]

8 heptadecan-9-yl 8-((O-((2-morpholinoethyl)carbamoyl)-N-palmitoylseryl)oxy)octanoate was synthesized according to representative synthetic scheme 1 and the corresponding methods. ¹H NMR (400 MHz, CDCl₃): δ 0.89 (9H, t, J=6.85 Hz), 1.27 (54H, m), 1.52 (2H, m), 1.65-1.70 (7H, m), 2.29 (2H, t, J=7.51 Hz), 2.37-2.43 (2H, m), 4.86 (2H, m), 2.83-2.97 (2H, m), 3.08-3.30 (2H, m), 3.56-3.77 (4H, m), 3.96-4.04 (2H, m), 4.15 (3H, m), 4.24-4.31 (1H, m), 4.34-4.45 (2H, m), 4.61 (1H, m), 6.83-6.91 (1H, m), 7.37 (1H, d, J=8.63 Hz). MS [ESI]: m/z: [M+H] calc. 880.7 obs. 881.9.

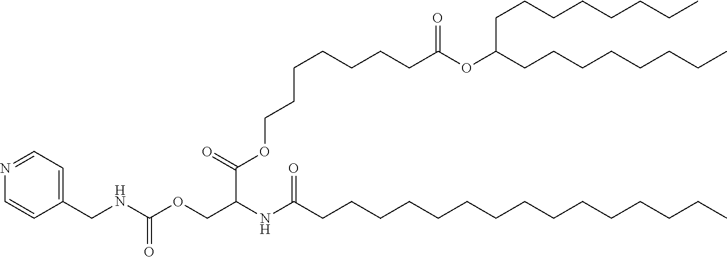 [SMILES: CCCCCCCCCCCCCCCC(=O)NC(COC(=O)NCc1ccncc1)C(=O)OCCCCCCCC(=O)OC(CCCCCCCC)CCCCCCCC]

heptadecan-9-yl 8-((N-palmitoyl-O-((pyridin-4-ylmethyl)carbamoyl)seryl)oxy)octanoate was synthesized according to representative synthetic scheme 1 and the corresponding methods. ¹H NMR (400 MHz, CDCl₃): δ 0.89 (9H, m), 1.27 (56H, m), 1.51 (3H, m), 1.65 (6H, m), 2.26 (3H, m), 4.16 (2H, m), 4.50 (4H, m), 4.85 (2H, m), 5.82 (1H, m), 6.47 (1H, d, J=7.52 Hz), 7.55 (2H, d, J=6.53 Hz), 8.62 (2H, d, J=5.86 Hz). MS [ESI]: m/z: [M+H] calc. 858.7 obs. 859.0.

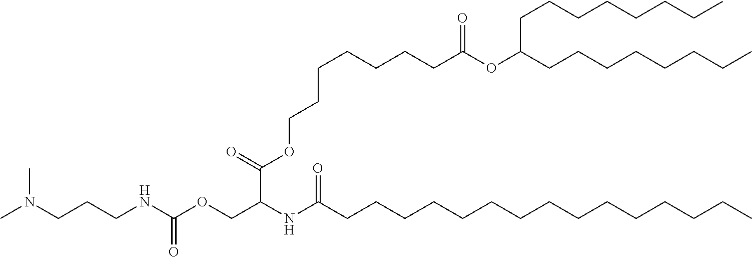 [SMILES: CCCCCCCCCCCCCCCC(=O)NC(COC(=O)NCCCN(C)C)C(=O)OCCCCCCCC(=O)OC(CCCCCCCC)CCCCCCCC]

heptadecan-9-yl 8-((O-((3-(dimethylamino)propyl)carbamoyl)-N-palmitoylseryl)oxy)octanoate was synthesized according to representative synthetic scheme 1 and the corresponding methods. ¹H NMR (400 MHz, CDCl₃): δ 0.87 (9H, t, J=6.67 Hz), 1.25 (53H, m), 1.47-1.53 (4H, m), 1.57-1.69 (8H, m), 2.28 (3H, m), 2.80 (6H, s), 1.99-2.13 (2H, m), 3.05-3.12 (2H, m), 3.32-3.39 (2H, m), 4.12 (2H, m), 4.44-4.49 (1H, m), 4.79-4.88 (2H, m), 4.26-4.32 (1H, m), 5.94-6.00 (1H, m), 6.61-6.66 (1H, m). MS [ESI]: m/z: [M+H] calc. 852.7 obs. 852.8.

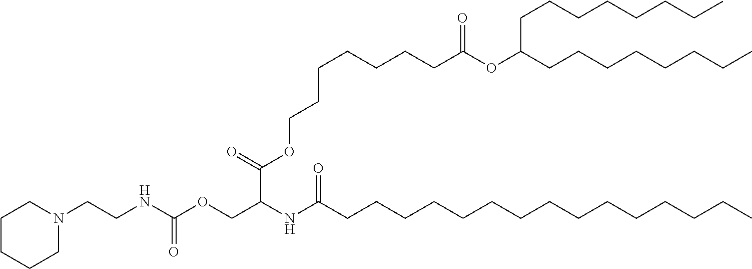 [SMILES: CCCCCCCCCCCCCCCC(=O)NC(COC(=O)NCCN1CCCCC1)C(=O)OCCCCCCCC(=O)OC(CCCCCCCC)CCCCCCCC]

heptadecan-9-yl 8-((N-palmitoyl-O-((2-(piperidin-1-yl)ethyl)carbamoyl)seryl)oxy)octanoate was synthesized according to representative synthetic scheme 1 and the corresponding methods. ¹H NMR (400 MHz, CDCl₃): δ 0.87 (9H, m), 1.25 (54H, m), 1.45-1.54 (5H, m), 1.63 (7H, br s), 2.27 (2H, t, J=7.53 Hz), 2.34-2.43 (4H, m), 1.81-1.96 (3H, m), 2.58-2.72 (2H, m), 3.00-3.20 (2H, m), 3.58 (1H, m), 3.71 (2H, m), 4.10 (2H, br s), 4.21-4.27 (1H, m), 4.59 (1H, dd, J=11.30, 3.37 Hz), 4.84 (2H, m), 6.98-7.03 (1H, m), 7.19-7.24 (1H, m). MS [ESI]: m/z: [M+H] calc. 878.7 obs. 878.8.

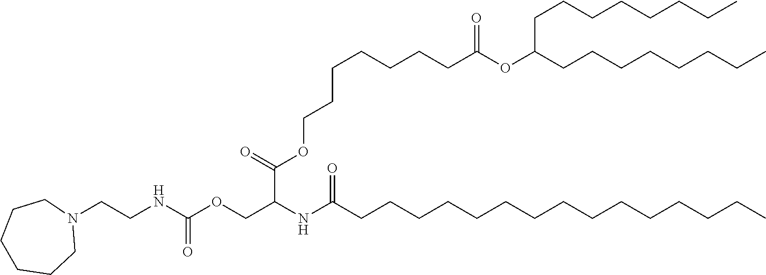 [SMILES: CCCCCCCCCCCCCCCC(=O)NC(COC(=O)NCCN1CCCCCC1)C(=O)OCCCCCCCC(=O)OC(CCCCCCCC)CCCCCCCC]

heptadecan-9-yl 8-((O-((2-(azepan-1-yl)ethyl)carbamoyl)-N-palmitoylseryl)oxy)octanoate was synthesized according to representative synthetic scheme 1 and the corresponding methods. ¹H NMR (400 MHz, CDCl₃): δ 0.88 (9H, m), 1.25 (56H, m), 1.45-1.77 (20H, m), 2.27 (2H, t, J=7.43 Hz), 2.32-2.37 (2H, m), 2.92-3.15 (4H, m), 3.44-3.57 (2H, m), 4.11 (2H, m), 4.22-4.31 (1H, m), 4.52-4.62 (1H, m)4.84 (2H, m). MS [ESI]: m/z: [M+H] calc. 892.8 obs. 893.8.

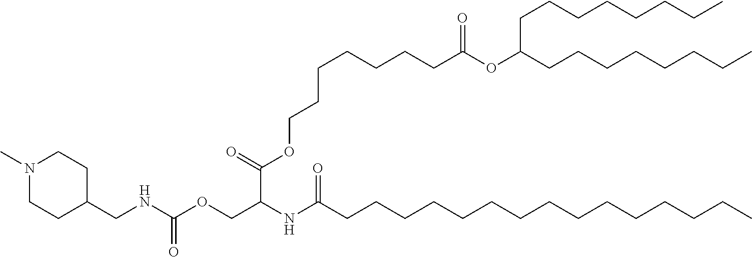 [SMILES: CCCCCCCCCCCCCCCC(=O)NC(COC(=O)NCC1CCN(C)CC1)C(=O)OCCCCCCCC(=O)OC(CCCCCCCC)CCCCCCCC]

heptadecan-9-yl 8-((O-(((1-methylpiperidin-4-yl)methyl)carbamoyl)-N-palmitoylseryl)oxy)octanoate was synthesized according to representative synthetic scheme 1 and the corresponding methods. ¹H NMR (400 MHz, CDCl₃): δ 0.87 (9H, t, J=6.78 Hz), 1.25 (54H, m), 1.47-1.53 (4H, m), 1.62 (8H, m), 2.25 (6H, m), 2.48 (3H, s), 1.71-1.80 (3H, m), 3.04-3.17 (4H, m), 4.13 (2H, t, J=6.68 Hz), 4.37 (2H, m), 4.82 (2H, m), 5.14 (1H, m), 6.46 (1H, m). MS [ESI]: m/z: [M+H] calc. 878.7 obs. 878.8.

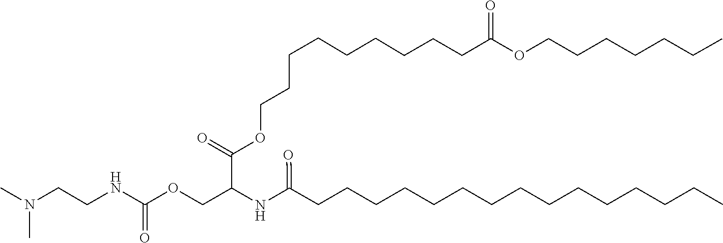 [SMILES: CCCCCCCCCCCCCCCC(=O)NC(COC(=O)NCCN(C)C)C(=O)OCCCCCCCCCC(=O)OCCCCCCC]

heptyl 10-((O-((2-(dimethylamino)ethyl)carbamoyl)-N-palmitoylseryl)oxy)decanoate was synthesized according to representative synthetic scheme 1 and the corresponding methods. ¹H NMR (400 MHz, CDCl₃): δ 0.88 (6H, m), 1.24 (42H, m), 1.63 (8H, m), 2.28 (2H, t, J=7.52 Hz), 2.31-2.36 (2H, m), 2.71 (5H, s), 2.87-3.09 (3H, m), 3.47 (2H, m), 4.05 (4H, m), 4.27-4.33 (1H, m), 4.50-4.57 (1H, m), 4.78-4.85 (1H, m), 6.24-6.41 (1H, m), 7.05-7.20 (1H, m). MS [ESI]: m/z: [M+H] calc. 726.6 obs. 726.4.

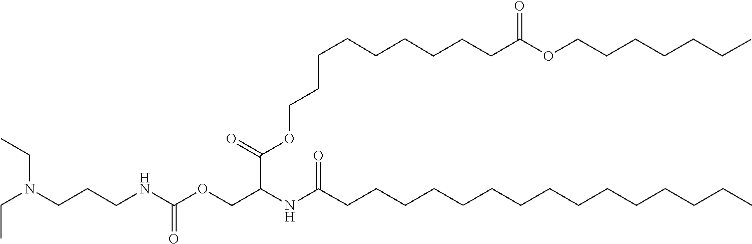 [SMILES: CCCCCCCCCCCCCCCC(=O)NC(COC(=O)NCCCN(CC)CC)C(=O)OCCCCCCCCCC(=O)OCCCCCCC]

heptyl 10-((O-((3-(diethylamino)propyl)carbamoyl)-N-palmitoylseryl)oxy)decanoate was synthesized according to representative synthetic scheme 1 and the corresponding methods. ¹H NMR (400 MHz, CDCl₃): δ 0.88 (6H, m), 1.26 (48H, m), 1.63 (9H, m), 1.82-1.93 (2H, m), 2.29 (4H, m), 2.75-2.92 (5H, m), 3.27-3.35 (2H, m), 4.06 (2H, t, J=6.75 Hz), 4.10-4.17 (2H, m), 4.27-4.34 (1H, m), 4.46 (1H, dd, J=11.27, 4.18 Hz), 4.76-4.82 (1H, m), 6.10-6.18 (1H, m), 6.49-6.56 (1H, m). MS [ESI]: m/z: [M+H] calc. 768.6 obs. 769.7.

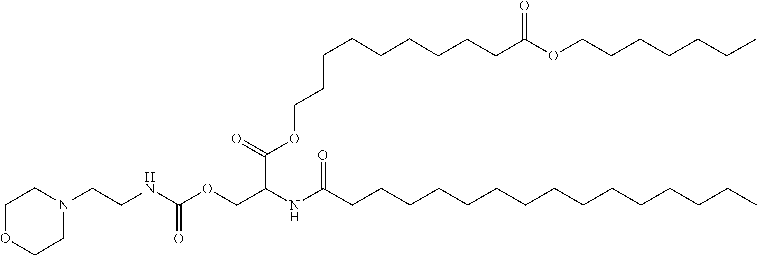 [SMILES: CCCCCCCCCCCCCCCC(=O)NC(COC(=O)NCCN1CCOCC1)C(=O)OCCCCCCCCCC(=O)OCCCCCCC]

heptyl 10-((O-((2-morpholinoethyl)carbamoyl)-N-palmitoylseryl)oxy)decanoate was synthesized according to representative synthetic scheme 1 and the corresponding methods. ¹H NMR (400 MHz, CDCl₃): δ 0.88 (6H, m), 1.25 (44H, m), 1.54-1.70 (13H, m), 2.28 (4H, m), 4.05 (5H, m), 4.26-4.32 (1H, m), 4.49-4.56 (1H, m), 4.79-4.86 (1H, m), 3.39-3.57 (2H, m), 3.78-4.00 (2H, m). MS [ESI]: m/z: [M+H] calc. 768.6 obs. 768.7.

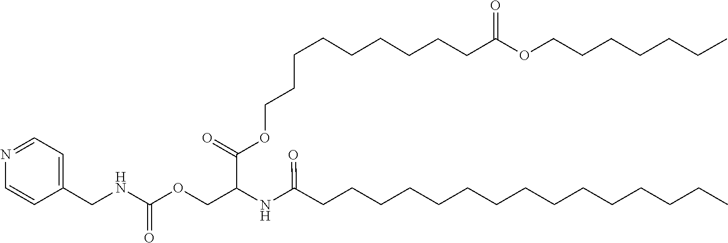 [SMILES: CCCCCCCCCCCCCCCC(=O)NC(COC(=O)NCc1ccncc1)C(=O)OCCCCCCCCCC(=O)OCCCCCCC]

heptyl 10-((N-palmitoyl-O-((pyridin-4-ylmethyl)carbamoyl)seryl)oxy)decanoate was synthesized according to representative synthetic scheme 1 and the corresponding methods. ¹H NMR (400 MHz, CDCl₃): δ 0.88 (6H, m), 1.25 (42H, m), 1.62 (7H, m), 2.27 (4H, m), 4.05 (2H, t, J=6.74 Hz), 4.09-4.17 (2H, m), 4.42-4.47 (2H, m), 4.51 (2H, d, J=6.15 Hz), 4.81-4.88 (1H, m), 5.68-5.74 (1H, m), 6.41 (1H, m), 7.56 (2H, s), 8.61 (2H, d, J=4.47 Hz). MS [ESI]: m/z: [M+H] calc. 746.6 obs. 746.6.

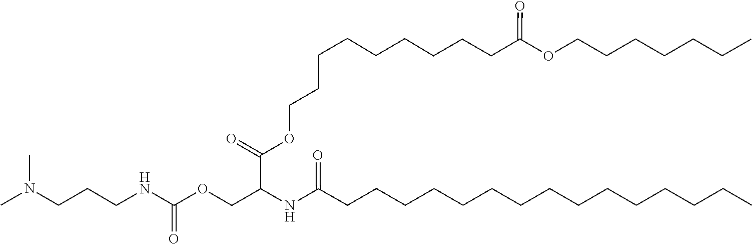 [SMILES: CCCCCCCCCCCCCCCC(=O)NC(COC(=O)NCCCN(C)C)C(=O)OCCCCCCCCCC(=O)OCCCCCCC]

heptyl 10-((O-((3-(dimethylamino)propyl)carbamoyl)-N-palmitoylseryl)oxy)decanoate was synthesized according to representative synthetic scheme 1 and the corresponding methods. ¹H NMR (400 MHz, CDCl₃): δ 0.89 (6H, m), 1.27 (42H, m), 1.58-1.69 (8H, m), 1.80-1.89 (2H, m), 2.24-2.33 (4H, m), 2.51 (5H, s), 2.70 (2H, m), 3.31 (2H, q, J=5.94 Hz), 4.07 (2H, t, J=6.74 Hz), 4.14 (2H, t, J=6.92 Hz), 4.30-4.36 (1H, m), 4.43-4.49 (1H, m), 4.78-4.85 (1H, m), 5.87 (1H, m), 6.54 (1H, m). MS [ESI]: m/z: [M+H] calc. 740.6 obs. 741.6.

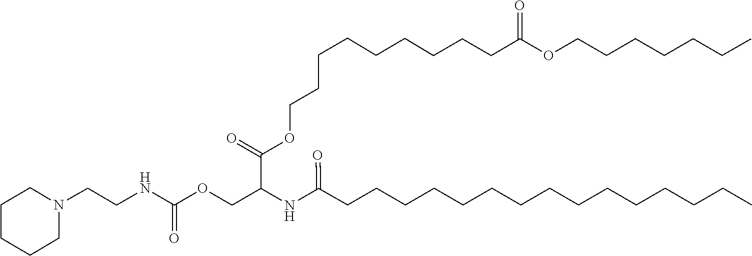 [SMILES: CCCCCCCCCCCCCCCC(=O)NC(COC(=O)NCCN1CCCCC1)C(=O)OCCCCCCCCCC(=O)OCCCCCCC]

heptyl 10-((N-palmitoyl-O-((2-(piperidin-1-yl)ethyl)carbamoyl)seryl)oxy)decanoate was synthesized according to representative synthetic scheme 1 and the corresponding methods. ¹H NMR (400 MHz, CDCl₃): δ 0.90 (6H, m), 1.27 (43H, m), 1.63 (14H, m), 2.30 (4H, m), 2.61 (5H, s), 3.35 (2H, m), 4.07 (2H, t, J=6.75 Hz), 4.12-4.18 (2H, 4.14 (t, J=7.10 Hz), 4.15 (t, J=6.73 Hz)), 4.33 (1H, dd, J=11.30, 2.88 Hz), 4.49 (1H, m), 4.79-4.84 (1H, 4.82 (d, J=14.90 Hz), 4.82 (t, J=3.99 Hz)), 6.65 (1H, br s). MS [ESI]: m/z: [M+H] calc. 766.6 obs. 766.3.

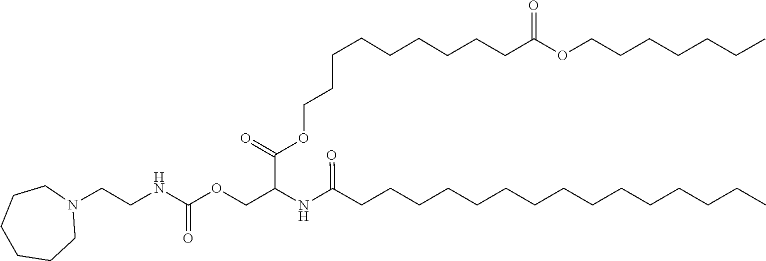 [SMILES: CCCCCCCCCCCCCCCC(=O)NC(COC(=O)NCCN1CCCCCC1)C(=O)OCCCCCCCCCC(=O)OCCCCCCC]

heptyl 10-((O-((2-(azepan-1-yl)ethyl)carbamoyl)-N-palmitoylseryl)oxy)decanoate was synthesized according to representative synthetic scheme 1 and the corresponding methods. ¹H NMR (400 MHz, CDCl₃): δ 0.90 (6H, m), 1.26 (42H, m), 1.64 (18H, m), 2.30 (4H, m), 2.80-3.25 (5H, m), 3.45 (2H, br s), 4.07 (2H, t, J=6.75 Hz), 4.10-4.18 (2H, m), 4.26-4.33 (1H, m), 4.53-4.59 (1H, m), 4.79-4.86 (1H, m). MS [ESI]: m/z: [M+H] calc. 780.6 obs. 780.6.

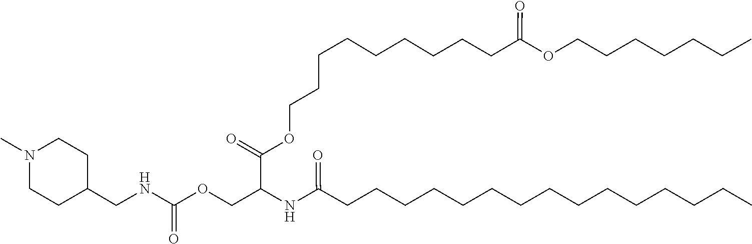 [SMILES: CCCCCCCCCCCCCCCC(=O)NC(COC(=O)NCC1CCN(C)CC1)C(=O)OCCCCCCCCCC(=O)OCCCCCCC]

heptyl 10-((O-(((1-methylpiperidin-4-yl)methyl)carbamoyl)-N-palmitoylseryl)oxy)decanoate was synthesized according to representative synthetic scheme 1 and the corresponding methods. ¹H NMR (400 MHz, CDCl₃): δ 0.88 (6H, m), 1.25 (43H, m), 1.62 (9H, m), 1.72-1.82 (2H, m), 2.26 (7H, m), 2.50 (3H, s), 3.13 (3H, m), 4.05 (2H, t, J=6.73 Hz), 4.14 (2H, m), 4.30-4.44 (2H, m), 4.79 (1H, m), 4.96-5.03 (1H, m), 6.41 (1H, m). MS [ESI]: m/z: [M+H] calc. 766.6 obs. 766.7.

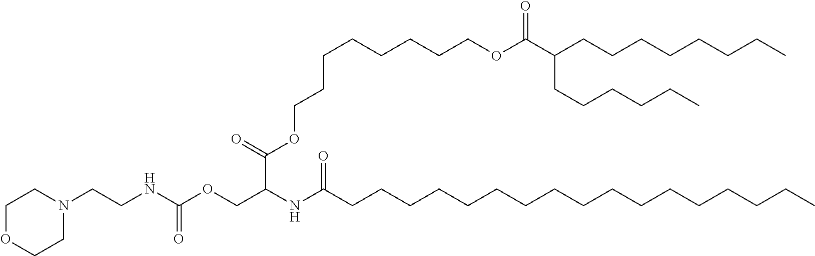 [SMILES: CCCCCCCCCCCCCCCCCC(=O)NC(COC(=O)NCCN1CCOCC1)C(=O)OCCCCCCCCOC(=O)C(CCCCCC)CCCCCCCC]

8-((O-((2-morpholinoethyl)carbamoyl)-N-stearoylseryl)oxy)octyl 2-hexyldecanoate was synthesized according to representative synthetic scheme 1 and the corresponding methods. ¹H NMR (400 MHz, CDCl₃): δ 0.85 (9H, m), 1.23 (59H, m), 1.52-1.66 (12H, m), 2.23-2.34 (4H, m), 3.36-3.52 (3H, m), 4.04 (7H, m), 4.25-4.32 (2H, m), 4.46-4.55 (2H, m), 4.76-4.83 (1H, m). MS [ESI]: m/z: [M+H] calc. 894.7 obs. 894.9.

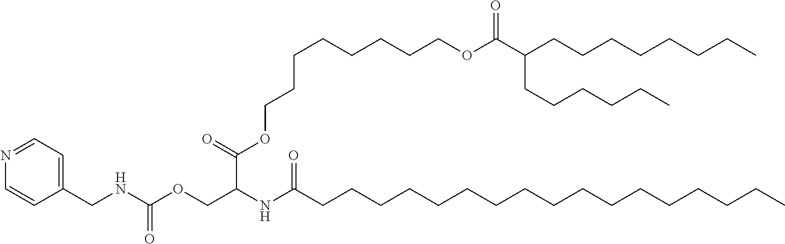 [SMILES: CCCCCCCCCCCCCCCCCC(=O)NC(COC(=O)NCc1ccncc1)C(=O)OCCCCCCCCOC(=O)C(CCCCCC)CCCCCCCC]

8-((O-((pyridin-4-ylmethyl)carbamoyl)-N-stearoylseryl)oxy)octyl 2-hexyldecanoate was synthesized according to representative synthetic scheme 1 and the corresponding methods. ¹H NMR (400 MHz, CDCl₃): δ 0.90 (9H, m), 1.27 (60H, m), 1.56-1.69 (8H, m), 2.27 (3H, m), 4.11 (5H, m), 4.43-4.49 (2H, m), 4.59 (2H, d, J=6.49 Hz), 4.85-4.90 (1H, m), 5.94-6.00 (1H, m), 6.45-6.50 (1H, m), 7.73 (1H, m), 8.64 (2H, d, J=5.82 Hz). MS [ESI]: m/z: [M+H] calc. 872.7 obs. 872.9.

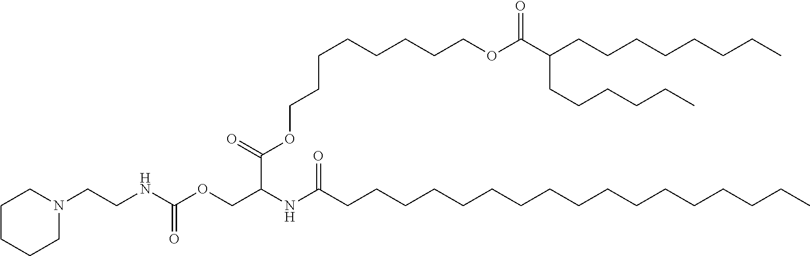 [SMILES: CCCCCCCCCCCCCCCCCC(=O)NC(COC(=O)NCCN1CCCCC1)C(=O)OCCCCCCCCOC(=O)C(CCCCCC)CCCCCCCC]

8-((O-((2-(piperidin-1-yl)ethyl)carbamoyl)-N-stearoylseryl)oxy)octyl 2-hexyldecanoate was synthesized according to representative synthetic scheme 1 and the corresponding methods. ¹H NMR (400 MHz, CDCl₃): δ 0.90 (9H, m), 1.27 (59H, m), 1.52-1.72 (11H, m), 1.85-2.03 (3H, m), 2.32 (3H, m), 2.76-2.99 (3H, m), 3.48 (2H, br s), 4.07 (5H, m), 4.27-4.32 (1H, m), 4.52-4.58 (1H, m), 4.83 (1H, m), 6.88-7.01 (1H, m). MS [ESI]: m/z: [M+H] calc. 892.8 obs. 892.9.

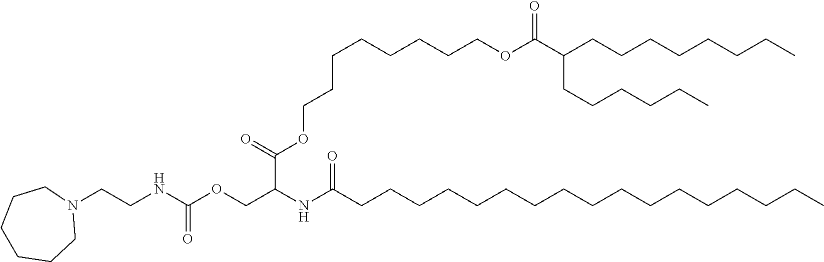 [SMILES: CCCCCCCCCCCCCCCCCC(=O)NC(COC(=O)NCCN1CCCCCC1)C(=O)OCCCCCCCCOC(=O)C(CCCCCC)CCCCCCCC]

8-((O-((2-(azepan-1-yl)ethyl)carbamoyl)-N-stearoylseryl)oxy)octyl 2-hexyldecanoate was synthesized according to representative synthetic scheme 1 and the corresponding methods. ¹H NMR (400 MHz, CDCl₃): δ 0.85 (9H, m), 1.23 (59H, m), 1.37-1.44 (2H, m), 1.51-1.58 (7H, m), 1.79-1.86 (3H, m), 2.10-2.19 (2H, m), 2.35 (2H, m), 2.91-2.99 (2H, m), 3.05-3.21 (3H, m), 3.51-3.56 (2H, m), 3.61-3.73 (3H, m), 4.04 (6H, s), 4.21-4.25 (1H, m), 4.54-4.59 (1H, m), 4.77-4.82 (1H, m). MS [ESI]: m/z: [M+H] calc. 906.8 obs. 907.0.

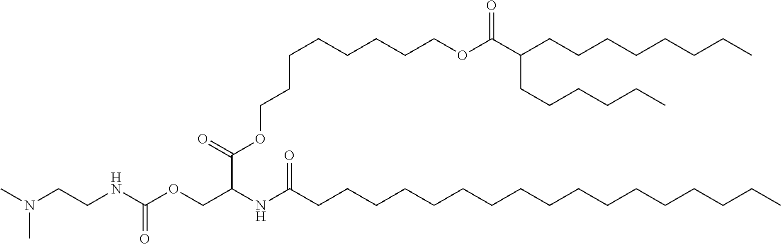 [SMILES: CCCCCCCCCCCCCCCCCC(=O)NC(COC(=O)NCCN(C)C)C(=O)OCCCCCCCCOC(=O)C(CCCCCC)CCCCCCCC]

2-methyl-6,10-dioxo-9-stearamido-7,11-dioxa-2,5-diazanonadecan-19-yl 2-hexyldecanoate was synthesized according to representative synthetic scheme 1 and the corresponding methods. ¹H NMR (400 MHz, CDCl₃): δ 0.85 (9H, s), 1.23 (56H, m), 1.36-1.44 (3H, m), 1.52-1.66 (10H, m), 2.25-2.34 (3H, m), 2.64 (5H, br s), 3.39-3.46 (2H, m), 4.04 (6H, m), 4.26-4.30 (1H, m), 4.48-4.53 (1H, m), 4.77-4.82 (1H, m). MS [ESI]: m/z: [M+H] calc. 852.7 obs. 852.9.

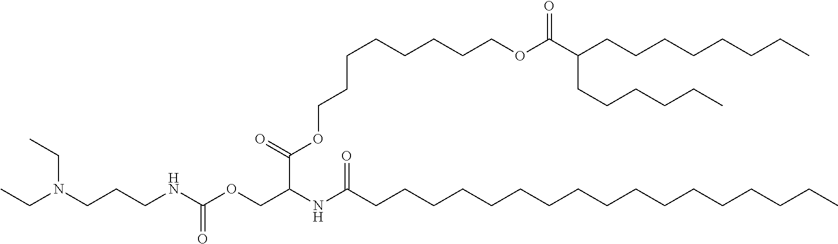 [SMILES: CCCCCCCCCCCCCCCCCC(=O)NC(COC(=O)NCCCN(CC)CC)C(=O)OCCCCCCCCOC(=O)C(CCCCCC)CCCCCCCC]

3-ethyl-8,12-dioxo-11-stearamido-9,13-dioxa-3,7-diazahenicosan-21-yl 2-hexyldecanoate was synthesized according to representative synthetic scheme 1 and the corresponding methods. ¹H NMR (400 MHz, CDCl₃): δ 0.85 (9H, s), 1.23 (53H, m), 1.37 (8H, m), 1.60-1.64 (2H, m), 2.01-2.08 (2H, m), 2.24-2.31 (8H, m), 3.00-3.08 (5H, m), 3.30-3.35 (3H, m), 4.04 (3H, t, J=6.40 Hz), 4.06-4.15 (4H, m), 4.244.27 (1H, m), 4.45 (1H, dd, J=11.28, 4.00 Hz), 4.76-4.81 (2H, m), 5.94-5.98 (1H, m), 6.57-6.61 (1, m). MS [ESI]: m/z: [M+H] calc. 894.8 obs. 895.9.

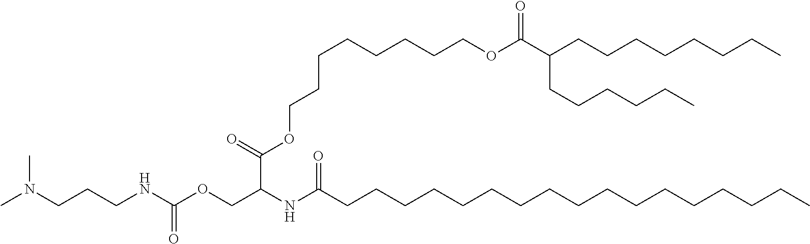 [SMILES: CCCCCCCCCCCCCCCCCC(=O)NC(COC(=O)NCCCN(C)C)C(=O)OCCCCCCCCOC(=O)C(CCCCCC)CCCCCCCC]

2-methyl-7,11-dioxo-10-stearamido-8,12-dioxa-2,6-diazaicosan-20-yl 2-hexyldecanoate was synthesized according to representative synthetic scheme 1 and the corresponding methods. ¹H NMR (400 MHz, CDCl₃): δ 0.85 (9H, m), 1.23 (54H, m), 1.36-1.44 (3H, m), 1.56-1.64 (5H, m), 2.00-2.09 (2H, m), 2.24-2.31 (3H, m), 2.79 (5H, s), 3.04-3.09 (2H, m), 3.29-3.37 (3H, m), 4.04 (3H, t, J=6.40 Hz), 4.07-4.15 (4H, m), 4.25-4.28 (1H, m), 4.43 (1H, dd, J=11.25, 4.05 Hz), 4.77-4.82 (2H, m), 5.93-5.98 (1H, m), 6.59-6.63 (1H, m). MS [ESI]: m/z: [M+H] calc. 866.7 obs. 867.0.

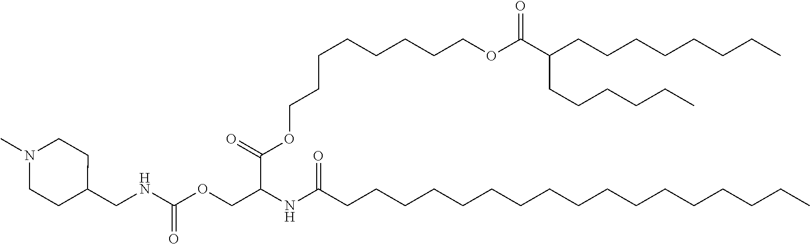 [SMILES: CCCCCCCCCCCCCCCCCC(=O)NC(COC(=O)NCC1CCN(C)CC1)C(=O)OCCCCCCCCOC(=O)C(CCCCCC)CCCCCCCC]

8-((O-(((1-methylpiperidin-4-yl)methyl)carbamoyl)-N-stearoylseryl)oxy)octyl 2-hexyldecanoate was synthesized according to representative synthetic scheme 1 and the corresponding methods. ¹H NMR (400 MHz, CDCl₃): δ 0.85 (9H, m), 1.23 (53H, m), 1.37-1.44 (2H, m), 1.53-1.56 (8H, m), 1.79-1.89 (2H, m), 1.98-2.06 (2H, m), 2.24 (3H, m), 2.61-2.67 (2H, m), 2.73 (3H, d, J=4.70 Hz), 3.02-3.09 (2H, m), 3.12-3.19 (1H, m), 3.47-3.54 (2H, m), 4.04 (3H, t, J=6.83 Hz), 4.08-4.17 (4H, m), 4.36 (2H, m), 4.76-4.80 (1H, m), 5.11-5.15 (1H, m), 6.44-6.48 (1H, m). MS [ESI]: m/z: [M+H] calc. 892.8 obs. 892.8.

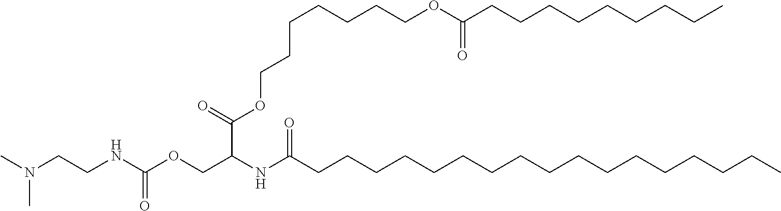 [SMILES: CCCCCCCCCCCCCCCCCC(=O)NC(COC(=O)NCCN(C)C)C(=O)OCCCCCCCOC(=O)CCCCCCCCC]

2-methyl-6,10-dioxo-9-stearamido-7,11-dioxa-2,5-diazaoctadecan-18-yl decanoate was synthesized according to representative synthetic scheme 1 and the corresponding methods. ¹H NMR (400 MHz, CDCl₃): δ 0.88 (6H, t, J=7.05 Hz), 1.25 (46H, m), 1.62 (8H, m), 2.22 (8H, m), 2.29 (2H, t, J=7.63 Hz), 2.39 (2H, t, J=6.07 Hz), 3.24 (2H, m), 4.05 (2H, t, J=6.70 Hz), 4.15 (2H, t, J=6.70 Hz), 4.34 (1H, dd, J=11.06, 2.93 Hz), 4.44 (1H, dd, J=11.06, 4.09 Hz), 4.80 (1H, m), 5.24 (1H, m), 6.38 (1H, d, J=8.37 Hz). MS [ESI]: m/z: [M+H] calc. 754.6 obs. 754.6.

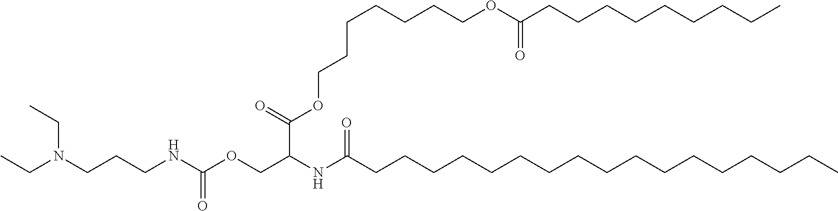 [SMILES: CCCCCCCCCCCCCCCCCC(=O)NC(COC(=O)NCCCN(CC)CC)C(=O)OCCCCCCCOC(=O)CCCCCCCCC]

8-((O-(((1-methylpiperidin-4-yl)methyl)carbamoyl)-N-stearoylseryl)oxy)octyl 2-hexyldecanoate was synthesized according to representative synthetic scheme 1 and the corresponding methods. ¹H NMR (400 MHz, CDCl₃): δ 0.88 (6H, t, J=6.93 Hz), 1.02 (6H, t, J=7.12 Hz), 1.25 (46H, m), 1.62 (10H, m), 2.22 (2H, t, J=7.81 Hz), 2.29 (2H, t, J=7.63 Hz), 2.47 (6H, m), 3.24 (2H, m), 4.05 (2H, t, J=6.70 Hz), 4.13 (2H, t, J=6.66 Hz), 4.33 (1H, dd, J=11.80, 3.07 Hz), 4.42 (1H, dd, J=11.80, 4.42 Hz), 4.77 (1H, m), 6.40 (2H, m). MS [ESI]: m/z: [M+H] calc. 796.7 obs. 797.6.

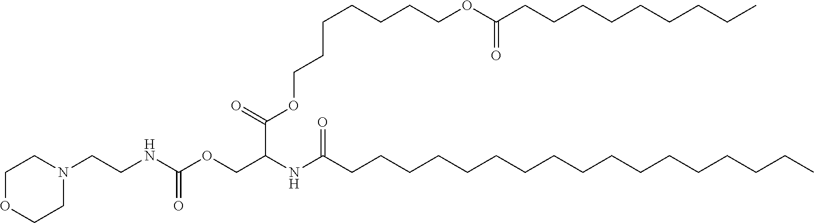 [SMILES: CCCCCCCCCCCCCCCCCC(=O)NC(COC(=O)NCCN1CCOCC1)C(=O)OCCCCCCCOC(=O)CCCCCCCCC]

7-((O-((2-morpholinoethyl)carbamoyl)-N-stearoylseryl)oxy)heptyl decanoate was synthesized according to representative synthetic scheme 1 and the corresponding methods. ¹H NMR (400 MHz, CDCl₃): δ 0.88 (6H, t, J=7.05 Hz), 1.25 (46H, m), 1.64 (8H, m), 2.24 (2H, t, J=7.79 Hz), 2.29 (2H, t, J=7.63 Hz), 2.45 (6H, m), 3.27 (2H, m), 3.70 (4H, t, J=4.32 Hz), 4.05 (2H, t, J=6.58 Hz), 4.15 (2H, m), 4.35 (1H, dd, J=11.58, 2.74 Hz), 4.45 (1H, dd, J=11.58, 4.37 Hz), 4.81 (1H, m), 5.19 (1H, m), 6.36 (1H, d, J=7.77 Hz). MS [ESI]: m/z: [M+H] calc. 796.6 obs. 796.7.

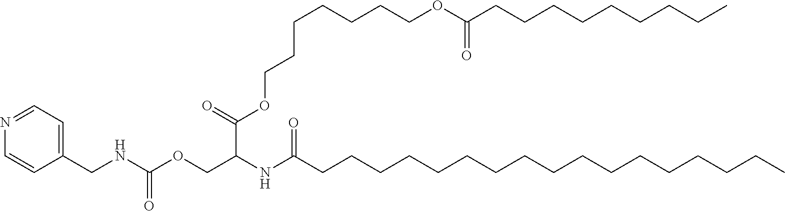 [SMILES: CCCCCCCCCCCCCCCCCC(=O)NC(COC(=O)NCc1ccncc1)C(=O)OCCCCCCCOC(=O)CCCCCCCCC]

7-((O-((pyridin-4-ylmethyl)carbamoyl)-N-stearoylseryl)oxy)heptyl decanoate was synthesized according to representative synthetic scheme 1 and the corresponding methods. ¹H NMR (400 MHz, CDCl₃): δ 0.88 (6H, t, J=6.86 Hz), 1.26 (46H, m), 1.62 (8H, m), 2.22 (2H, t, J=7.81 Hz), 2.28 (2H, t, J=7.64 Hz), 4.05 (2H, t, J=6.70 Hz), 4.15 (2H, m), 4.37 (2H, d, J=6.16 Hz), 4.42 (1H, dd, J=11.78, 3.24 Hz), 4.49 (1H, dd, J=11.78, 4.69 Hz), 4.83 (1H, m), 5.21 (1H, t, J=6.86 Hz), 6.31 (1H, d, J=8.07 Hz), 7.19 (2H, d, J=5.99 Hz), 8.57 (2H d, J=5.58 Hz). MS [ESI]: m/z: [M+H] calc. 774.6 obs. 774.7.

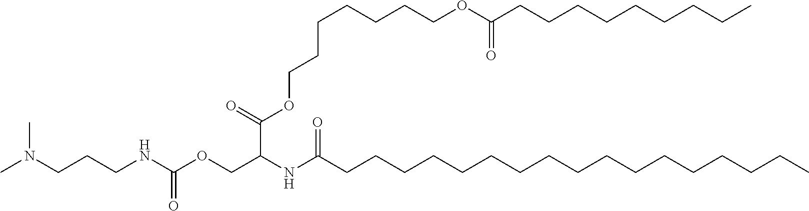 [SMILES: CCCCCCCCCCCCCCCCCC(=O)NC(COC(=O)NCCCN(C)C)C(=O)OCCCCCCCOC(=O)CCCCCCCCC]

2-methyl-7,11-dioxo-10-stearamido-8,12-dioxa-2,6-diazanonadecan-19-yl decanoate was synthesized according to representative synthetic scheme 1 and the corresponding methods. ¹H NMR (400 MHz, CDCl₃): δ 0.88 (6H, t, J=7.05 Hz), 1.25 (46H, m), 1.63 (10H, m), 2.22 (8H, m), 2.29 (2H, t, J=7.61 Hz), 2.34 (2H, t, J=6.61 Hz), 3.23 (2H, m), 4.05 (2H, t, J=6.70 Hz), 4.14 (2H, t, J=6.70 Hz), 4.33 (1H, dd, J=11.38, 3.19 Hz), 4.43 (1H, dd, J=11.38, 4.45 Hz), 4.79 (1H, m), 5.73 (1H, t, J=5.88 Hz), 6.42 (1H, d, J=7.72 Hz). MS [ESI]: m/z: [M+H] calc. 768.6 obs. 768.8.

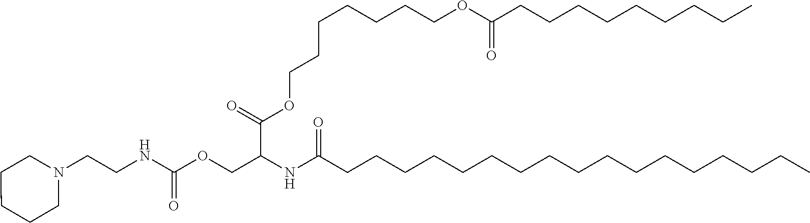 [SMILES: CCCCCCCCCCCCCCCCCC(=O)NC(COC(=O)NCCN1CCCCC1)C(=O)OCCCCCCCOC(=O)CCCCCCCCC]

7-((O-((2-(piperidin-1-yl)ethyl)carbamoyl)-N-stearoylseryl)oxy)heptyl decanoate was synthesized according to representative synthetic scheme 1 and the corresponding methods. ¹H NMR (400 MHz, CDCl₃): δ 0.88 (6H, t, J=7.16 Hz), 1.25 (46H, m), 1.46 (2H, m), 1.62 (12H, m), 2.29 (10H, m), 3.28 (2H, m), 4.05 (2H, t, J=6.70 Hz), 4.15 (2H, t, J=6.70 Hz), 4.33 (1H, dd, J=11.31, 2.71 Hz), 4.46 (1H, dd, J=11.31, 4.43 Hz), 4.80 (1H, m), 5.32 (1H, m), 6.46 (1H, m). MS [ESI]: m/z: [M+H] calc. 794.6 obs. 794.6.

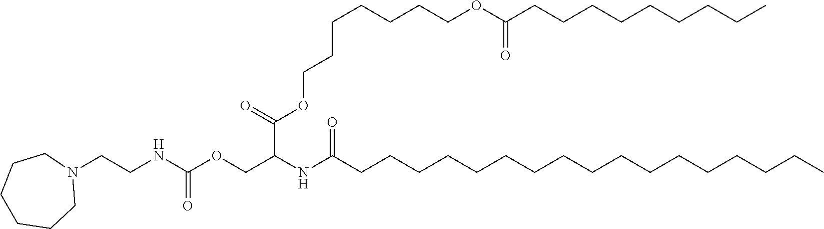 [SMILES: CCCCCCCCCCCCCCCCCC(=O)NC(COC(=O)NCCN1CCCCCC1)C(=O)OCCCCCCCOC(=O)CCCCCCCCC]

7-((O-((2-(azepan-1-yl)ethyl)carbamoyl)-N-stearoylseryl)oxy)heptyl decanoate was synthesized according to representative synthetic scheme 1 and the corresponding methods. ¹H NMR (400 MHz, CDCl₃): δ 0.88 (6H, t, J=6.66 Hz), 1.25 (46H, m), 1.60 (16H, m), 2.29 (4H, m), 2.61 (6H, m), 3.20 (2H, m), 4.05 (2H, t, J=6.70 Hz), 4.15 (2H, t, J=6.75 Hz), 4.34 (1H, dd, J=11.13, 3.06 Hz), 4.45 (1H, dd, J=11.13, 4.42 Hz), 4.79 (1H, m), 5.32 (1H, m), 6.42 (1H, d, J=7.89 Hz). MS [ESI]: m/z: [M+H] calc. 808.7 obs. 808.7.

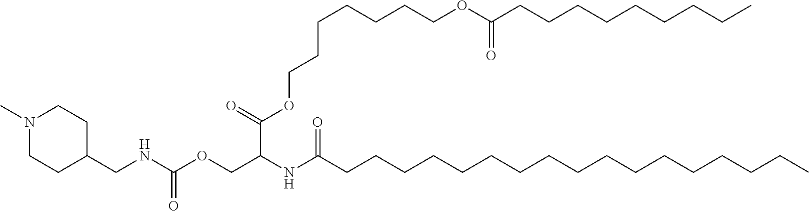 [SMILES: CCCCCCCCCCCCCCCCCC(=O)NC(COC(=O)NCC1CCN(C)CC1)C(=O)OCCCCCCCOC(=O)CCCCCCCCC]

7-((O-(((1-methylpiperidin-4-yl)methyl)carbamoyl)-N-stearoylseryl)oxy)heptyl decanoate was synthesized according to representative synthetic scheme 1 and the corresponding methods. ¹H NMR (400 MHz, CDCl₃): δ 0.88 (6H, t, J=6.98 Hz), 1.25 (46H, m), 1.63 (13H, m), 1.90 (2H, t, J=11.47 Hz), 2.26 (7H, m), 2.85 (2H, d, J=11.55 Hz), 3.06 (2H, t, J=6.24 Hz), 4.05 (2H, t, J=6.66 Hz), 4.14 (2H, td, J=6.83, 1.42 Hz), 4.34 (1H, dd, J=11.41, 3.24 Hz), 4.43 (1H, dd, J=11.41, 4.32 Hz), 4.80 (2H, m), 6.33 (1H, d, J=7.69 Hz). MS [ESI]: m/z: [M+H] calc. 794.7 obs. 794.7.

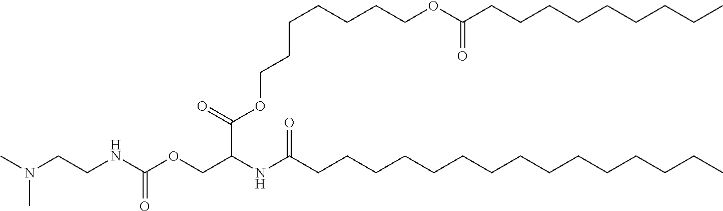 [SMILES: CCCCCCCCCCCCCCCC(=O)NC(COC(=O)NCCN(C)C)C(=O)OCCCCCCCOC(=O)CCCCCCCCC]

7-((O-(((1-methylpiperidin-4-yl)methyl)carbamoyl)-N-stearoylseryl)oxy)heptyl decanoate was synthesized according to representative synthetic scheme 1 and the corresponding methods. ¹H NMR (400 MHz, CDCl₃): δ 0.88 (6H, t, J=6.83 Hz), 1.25 (42H, m), 1.63 (8H, m), 2.22 (8H, m), 2.29 (2H, t, J=7.43 Hz), 2.39 (2H, t, J=6.16 Hz), 3.23 (2H, m), 4.05 (2H, t, J=6.70 Hz), 4.15 (2H, t, J=6.72 Hz), 4.34 (1H, dd, J=11.48, 2.71 Hz), 4.44 (1H, dd, J=11.48, 4.17 Hz), 4.80 (1H, m), 5.30 (1H, m), 6.38 (1H, d, J=7.78 Hz). MS [ESI]: m/z: [M+H] calc. 726.6 obs. 726.8.

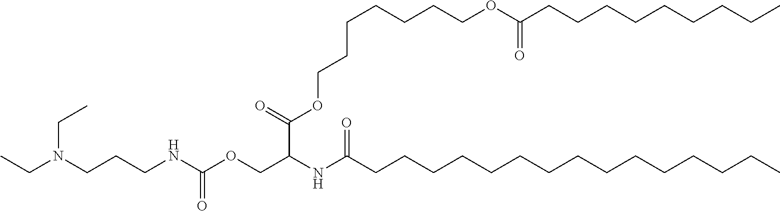 [SMILES: CCCCCCCCCCCCCCCC(=O)NC(COC(=O)NCCCN(CC)CC)C(=O)OCCCCCCCOC(=O)CCCCCCCCC]

3-ethyl-8,12-dioxo-11-palmitamido-9,13-dioxa-3,7-diazaicosan-20-yl decanoate was synthesized according to representative synthetic scheme 1 and the corresponding methods. ¹H NMR (400 MHz, CDCl₃): δ 0.88 (6H, t, J=7.02 Hz), 1.02 (6H, t, J=7.13 Hz), 1.26 (42H, m), 1.63 (10H, m), 2.23 (2H, t, J=7.80 Hz), 2.29 (2H, t, J=7.43 Hz), 2.47 (6H, m), 3.24 (2H, m), 4.05 (2H, t, J=6.66 Hz), 4.13 (2H, t, J=6.95 Hz), 4.33 (1H, dd, J=11.52, 3.03 Hz), 4.42 (1H, dd, J=11.52, 4.66 Hz), 4.76 (1H, m), 6.41 (2H, m). MS [ESI]: m/z: [M+H] calc. 768.6 obs. 768.7.

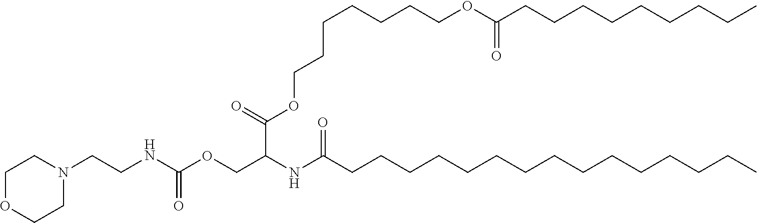 [SMILES: CCCCCCCCCCCCCCCC(=O)NC(COC(=O)NCCN1CCOCC1)C(=O)OCCCCCCCOC(=O)CCCCCCCCC]

7-((O-((2-morpholinoethyl)carbamoyl)-N-palmitoylseryl)oxy)heptyl decanoate was synthesized according to representative synthetic scheme 1 and the corresponding methods. ¹H NMR (400 MHz, CDCl₃): δ 0.88 (6H, t, J=6.93 Hz), 1.25 (42H, m), 1.63 (8H, m), 2.23 (2H, t, J=7.70 Hz), 2.29 (2H, t, J=7.44 Hz), 2.44 (6H, m), 3.26 (2H, m), 3.70 (4H, t, J=4.62 Hz), 4.05 (2H, t, J=6.70 Hz), 4.15 (2H, m), 4.35 (1H, dd, J=11.46, 2.50 Hz), 4.45 (1H, dd, J=11.46, 4.37 Hz), 4.80 (1H, m), 5.20 (1H, m), 6.37 (1H, d, J=7.82 Hz). MS [ESI]: m/z: [M+H] calc. 768.6 obs. 768.6.

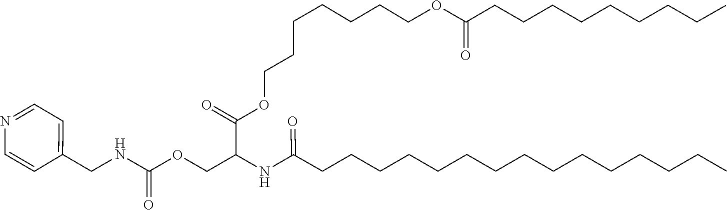 [SMILES: CCCCCCCCCCCCCCCC(=O)NC(COC(=O)NCc1ccncc1)C(=O)OCCCCCCCOC(=O)CCCCCCCCC]

7-((N-palmitoyl-O-((pyridin-4-ylmethyl)carbamoyl)seryl)oxy)heptyl decanoate was synthesized according to representative synthetic scheme 1 and the corresponding methods. ¹H NMR (400 MHz, CDCl₃): δ 0.88 (6H, t, J=6.96 Hz), 1.25 (42H, m), 1.61 (8H, m), 2.22 (2H, t, J=7.70 Hz), 2.28 (2H, t, J=7.72 Hz), 4.05 (2H, t, J=6.79 Hz), 4.14 (2H, m), 4.37 (2H, d, J=6.31 Hz), 4.42 (1H, dd, J=11.56, 3.42 Hz), 4.49 (1H, dd, J=11.56, 4.52 Hz), 4.82 (1H, m), 5.24 (1H, t, J=6.21 Hz), 6.31 (1H, d, J=7.64 Hz), 7.19 (2H, d, J=5.82 Hz), 8.57 (2H, d, J=5.60 Hz). MS [ESI]: m/z: [M+H] calc. 746.6 obs. 746.5.

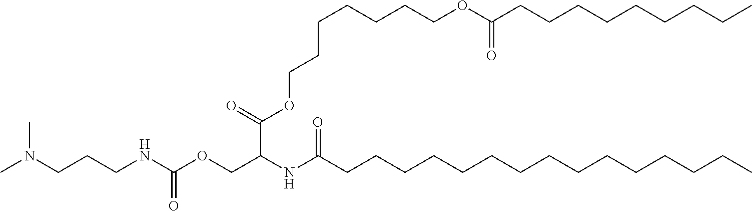 [SMILES: CCCCCCCCCCCCCCCC(=O)NC(COC(=O)NCCCN(C)C)C(=O)OCCCCCCCOC(=O)CCCCCCCCC]

2-methyl-7,11-dioxo-10-palmitamido-8,12-dioxa-2,6-diazanonadecan-19-yl decanoate was synthesized according to representative synthetic scheme 1 and the corresponding methods. ¹H NMR (400 MHz, CDCl₃): δ 0.88 (6H, t, J=6.98 Hz), 1.25 (42H, m), 1.63 (10H, m), 2.21 (12H, m), 3.23 (2H, m), 4.05 (2H, t, J=6.70 Hz), 4.14 (2H, t, J=6.71 Hz), 4.33 (1H, dd, J=11.51, 3.06 Hz), 4.43 (1H, dd, J=11.51, 4.52 Hz), 4.78 (1H, m), 5.74 (1H, m), 6.42 (1H, d, J=7.64 Hz). MS [ESI]: m/z: [M+H] calc. 740.6 obs. 740.8.

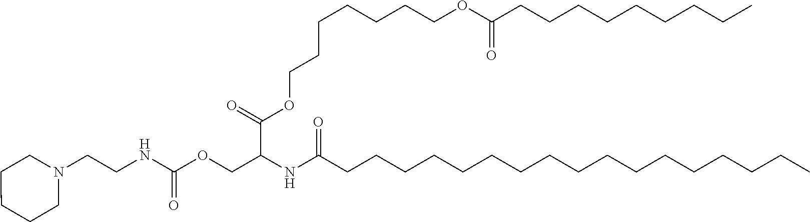 [SMILES: CCCCCCCCCCCCCCCCCC(=O)NC(COC(=O)NCCN1CCCCC1)C(=O)OCCCCCCCOC(=O)CCCCCCCCC]

7-((N-palmitoyl-O-((2-(piperidin-1-yl)ethyl)carbamoyl)seryl)oxy)heptyl decanoate was synthesized according to representative synthetic scheme 1 and the corresponding methods. ¹H NMR (400 MHz, CDCl₃): δ 0.88 (6H, t, J=7.17 Hz), 1.25 (44H, m), 1.61 (12H, m), 2.24 (2H, t, J=7.66 Hz), 2.29 (2H, t, J=7.64 Hz), 2.38 (6H, m), 3.24 (2H, m), 4.05 (2H, t, J=6.70 Hz), 4.15 (2H, t, J=6.70 Hz), 4.33 (1H, dd, J=11.60, 3.13 Hz), 4.45 (1H, dd, J=11.60, 4.44 Hz), 4.80 (1H, m), 5.30 (1H, m), 6.40 (1H, d, J=7.94 Hz). MS [ESI]: m/z: [M+H] calc. 766.6 obs. 766.7.

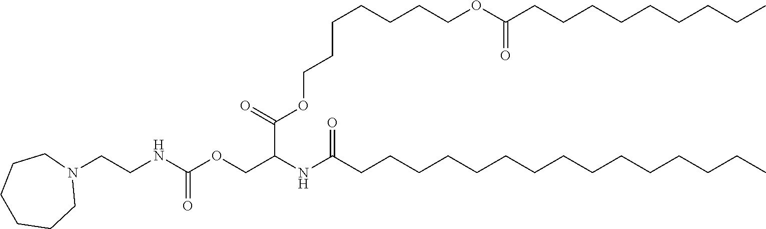 [SMILES: CCCCCCCCCCCCCCCC(=O)NC(COC(=O)NCCN1CCCCCC1)C(=O)OCCCCCCCOC(=O)CCCCCCCCC]

7-((O-((2-(azepan-1-yl)ethyl)carbamoyl)-N-palmitoylseryl)oxy)heptyl decanoate was synthesized according to representative synthetic scheme 1 and the corresponding methods. δ 0.88 (6H, t, J=7.07 Hz), 1.26 (42H, m), 1.60 (16H, m), 2.24 (2H, t, J=7.75 Hz), 2.29 (2H, t, J=7.43 Hz), 2.62 (6H, m), 3.19 (2H, m), 4.05 (2H, t, J=6.71 Hz), 4.15 (2H, t, J=6.71 Hz), 4.34 (1H, dd, J=11.63, 2.85 Hz), 4.45 (1H, dd, J=11.63, 4.37 Hz), 4.80 (1H, m), 5.30 (1H, m), 6.40 (1H, d, J=7.87 Hz). MS [ESI]: m/z: [M+H] calc. 780.6 obs. 781.7.

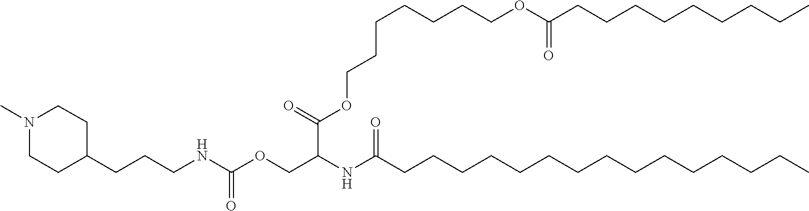 [SMILES: CCCCCCCCCCCCCCCC(=O)NC(COC(=O)NCCCC1CCN(C)CC1)C(=O)OCCCCCCCOC(=O)CCCCCCCCC]

7-((O-(((1-methylpiperidin-4-yl)methyl)carbamoyl)-N-palmitoylseryl)oxy)heptyl decanoate was synthesized according to representative synthetic scheme 1 and the corresponding methods. ¹H NMR (400 MHz, CDCl₃): δ 0.88 (6H, t, J=6.99 Hz), 1.25 (44H, m), 1.63 (11H, m), 1.89 (2H, td, J=11.65, 2.13 Hz), 2.26 (7H, m), 2.84 (2H, d, J=11.65 Hz), 3.07 (2H, t, J=6.47 Hz), 4.05 (2H, t, J=6.70 Hz), 4.15 (2H, td, J=6.83, 1.45 Hz), 4.34 (1H, dd, J=11.32, 3.19 Hz), 4.43 (1H, dd, J=11.32, 4.23 Hz), 4.80 (2H, m), 6.34 (1H, d, J=7.75 Hz). MS [ESI]: m/z: [M+H] calc. 766.6 obs. 766.6.

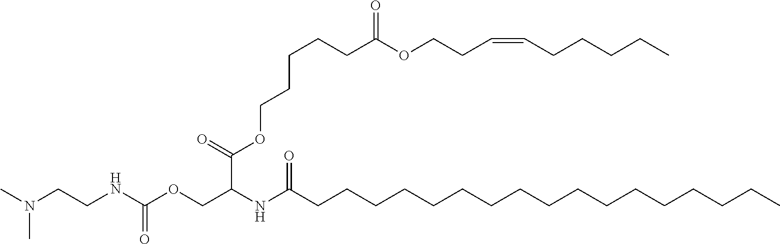 [SMILES: CCCCC/C=C\CCOC(=O)CCCCCOC(=O)C(COC(=O)NCCN(C)C)NC(=O)CCCCCCCCCCCCCCCCC]

(Z)-non-3-en-1-yl 6-((O-((2-(dimethylamino)ethyl)carbamoyl)-N-stearoylseryl)oxy)hexanoate was synthesized according to representative synthetic scheme 1 and the corresponding methods. ¹H NMR (400 MHz, CDCl₃): δ 0.88 (6H, m), 1.25 (36H, m), 1.66 (6H, m), 2.03 (2H, q, J=7.24 Hz), 2.22 (8H, m), 2.31 (2H, t, J=7.46 Hz), 2.38 (4H, m), 3.23 (2H, m), 4.06 (2H, t, J=6.96 Hz), 4.16 (2H, td, J=6.79, 1.52 Hz), 4.34 (1H, dd, J=11.43, 2.62 Hz), 4.43 (1H, dd, J=11.43, 3.94 Hz), 4.79 (1H, m), 5.33 (2H, m), 5.50 (1H, m), 6.42 (1H, d, J=7.82 Hz). MS [ESI]: m/z: [M+H] calc. 724.6 obs. 724.7.

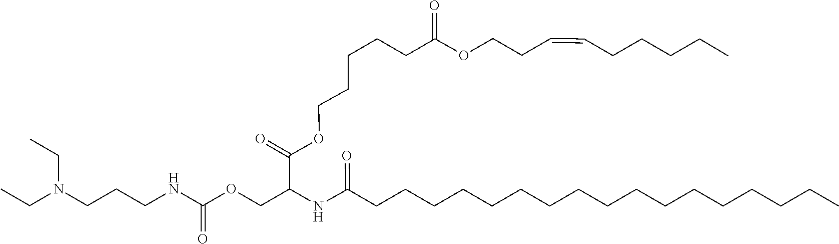 [SMILES: CCCCC/C=C\CCOC(=O)CCCCCOC(=O)C(COC(=O)NCCCN(CC)CC)NC(=O)CCCCCCCCCCCCCCCCC]

(Z)-non-3-en-1-yl 6-((O-((3-(diethylamino)propyl)carbamoyl)-N-stearoylseryl)oxy)hexanoate was synthesized according to representative synthetic scheme 1 and the corresponding methods. ¹H NMR (400 MHz, CDCl₃): δ 0.88 (6H, m), 1.02 (6H, t, J=7.26 Hz), 1.25 (36H, m), 1.58-1.71 (8H, m), 2.03 (2H, q, J=7.20 Hz), 2.22 (2H, t, J=7.75 Hz), 2.30 (2H, t, J=7.66 Hz), 2.37 (2H, q, J=7.34 Hz), 2.48 (6H, m), 3.23 (2H, m), 4.06 (2H, t, J=6.96 Hz), 4.14 (2H, t, J=6.66 Hz), 4.34 (1H, dd, J=11.83, 2.79 Hz), 4.41 (1H, dd, J=11.83, 4.25 Hz), 4.77 (1H, m), 5.32 (1H, m), 5.49 (1H, m), 6.39 (1H, m), 6.45 (1H, d, J=7.89 Hz). MS [ESI]: m/z: [M+H] calc. 766.3 obs. 768.8.

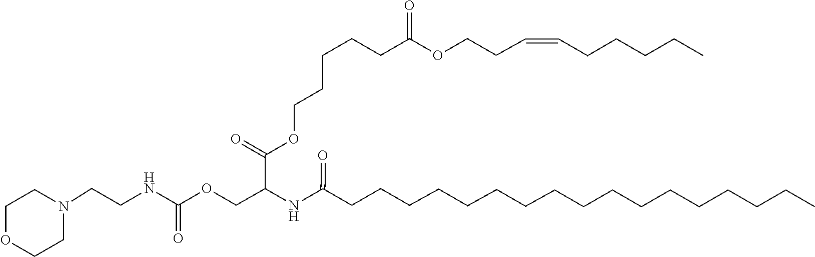 [SMILES: CCCCC/C=C\CCOC(=O)CCCCCOC(=O)C(COC(=O)NCCN1CCOCC1)NC(=O)CCCCCCCCCCCCCCCCC]

(Z)-non-3-en-1-yl 6-((O-((2-morpholinoethyl)carbamoyl)-N-stearoylseryl)oxy)hexanoate was synthesized according to representative synthetic scheme 1 and the corresponding methods. ¹H NMR (400 MHz, CDCl₃): δ 0.88 (6H, m), 1.25 (36H, m), 1.66 (6H, m), 2.03 (2H, q, J=7.29 Hz), 2.23 (2H, t, J=7.51 Hz), 2.31 (211, t, J=7.54 Hz), 2.37 (2H, q, J=7.16 Hz), 2.46 (6H, m), 3.27 (211, m), 3.71 (411, t, J=4.48 Hz), 4.06 (2H, t, J=6.86 Hz), 4.17 (2H, t, J=6.55 Hz), 4.36 (1H, dd, J=11.56, 2.51 Hz), 4.44 (1H, dd, J=11.56, 4.12 Hz), 4.80 (111, m), 5.31 (211, m), 5.50 (1H, m), 6.41 (1H, d, J=7.77 Hz). MS [ESI]: m/z: [M+H] calc. 766.6 obs. 766.5.

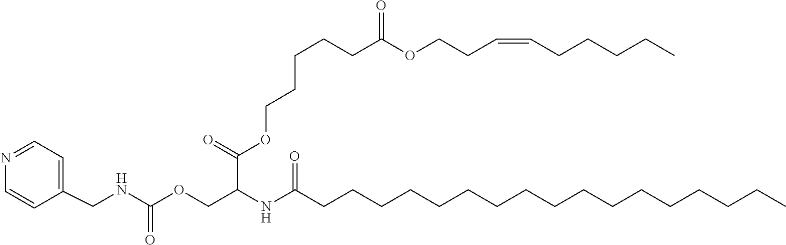 [SMILES: CCCCC/C=C\CCOC(=O)CCCCCOC(=O)C(COC(=O)NCc1ccncc1)NC(=O)CCCCCCCCCCCCCCCCC]

(Z)-non-3-en-1-yl 6-((O-((pyridin-4-ylmethyl)carbamoyl)-N-stearoylseryl)oxy)hexanoate was synthesized according to representative synthetic scheme 1 and the corresponding methods. ¹H NMR (400 MHz, CDCl₃): δ 0.88 (6H, m), 1.25 (36H, m), 1.63 (6H, m), 2.02 (2H, q, J=7.56 Hz), 2.22 (2H, t, J=7.71 Hz), 2.29 (2H, t, J=7.33 Hz), 2.35 (2H, q, J=7.04 Hz), 4.02 (2H, t, J=7.02 Hz), 4.15 (2H, m), 4.37 (2H, d, J=6.07 Hz), 4.45 (2H, d, J=3.53 Hz), 4.82 (1H, m), 5.31 (1H, m), 5.52 (2H, m), 6.39 (1H, d, J=7.68 Hz), 7.19 (2H, d, J=5.93 Hz), 8.56 (2H, d, J=5.23 Hz). MS [ESI]: m/z: [M+H] calc. 744.5 obs. 745.5.

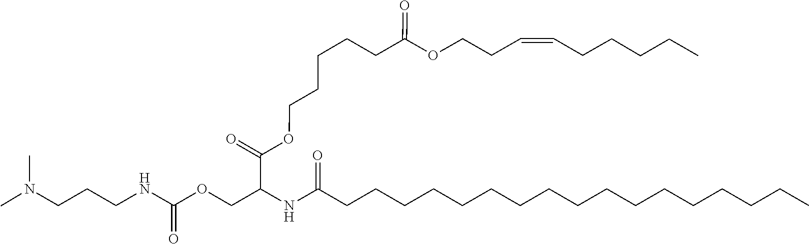 [SMILES: CCCCC/C=C\CCOC(=O)CCCCCOC(=O)C(COC(=O)NCCCN(C)C)NC(=O)CCCCCCCCCCCCCCCCC]

(Z)-non-3-en-1-yl6-((O-((3-(dimethylamino)propyl)carbamoyl)-N-stearoylseryl)oxy)hexanoate was synthesized according to representative synthetic scheme 1 and the corresponding methods. ¹H NMR (400 MHz, CDCl₃): δ 0.88 (6H, m), 1.25 (36H, m), 1.65 (8H, m), 2.03 (2H, q, J=7.26 Hz), 2.23 (8H, m), 2.31 (2H, t, J=7.55 Hz), 2.36 (4H, m), 3.23 (2H, m), 4.06 (2H, t, J=6.96 Hz), 4.16 (2H, m), 4.34 (1H, dd, J=11.63, 3.20 Hz), 4.42 (1H, dd, J=11.63, 4.35 Hz), 4.78 (1H, m), 5.33 (1H, m), 5.50 (1H, m), 5.78 (1H, t, J=5.73 Hz), 6.47 (1H, d, J=7.69 Hz). MS [ESI]: m/z: [M+H] calc. 738.6 obs. 738.6.

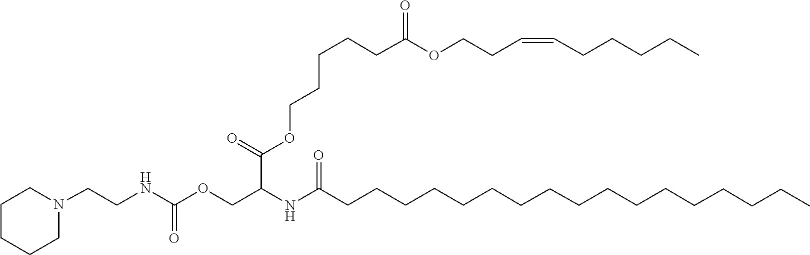 [SMILES: CCCCC/C=C\CCOC(=O)CCCCCOC(=O)C(COC(=O)NCCN1CCCCC1)NC(=O)CCCCCCCCCCCCCCCCC]

(Z)-non-3-en-1-yl 6-((O-((2-(piperidin-1-yl)ethyl)carbamoyl)-N-stearoylseryl)oxy)hexanoate was synthesized according to representative synthetic scheme 1 and the corresponding methods. MS [ESI]: m/z: [M+H]. calc. 764.6 obs. 764.7; ¹H NMR (400 MHz, CDCl₃): δ 0.88 (6H, m), 1.25 (36H, m), 1.65 (12H, m), 2.03 (2H, q, J=7.52 Hz), 2.24 (2H, t, J=7.78 Hz), 2.36 (10H, m), 3.24 (2H, m), 4.06 (2H, t, J=7.00 Hz), 4.16 (2H, t, J=6.58 Hz), 4.34 (1H, dd, J=11.64, 2.85 Hz), 4.44 (1H, dd, J=11.64, 4.27 Hz), 4.80 (1H, m), 5.34 (2H, m), 5.50 (1H, m), 6.44 (1H, d, J=7.86 Hz).

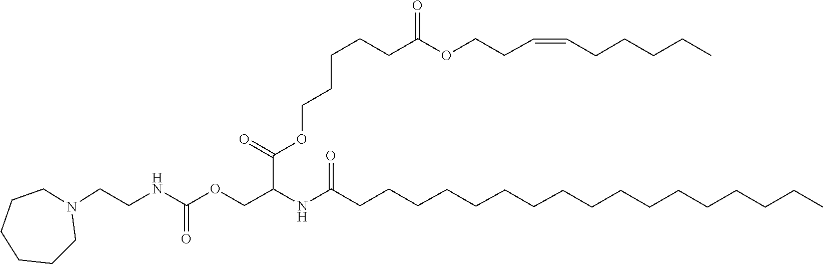 [SMILES: CCCCC/C=C\CCOC(=O)CCCCCOC(=O)C(COC(=O)NCCN1CCCCCC1)NC(=O)CCCCCCCCCCCCCCCCC]

(Z)-non-3-en-1-yl 6-((O-((2-(azepan-1-yl)ethyl)carbamoyl)-N-stearoylseryl)oxy)hexanoate was synthesized according to representative synthetic scheme 1 and the corresponding methods. MS [ESI]: m/z: [M+H]. calc. 778.6 obs. 779.1. ¹H NMR (400 MHz, CDCl₃): δ 0.88 (6H, m), 1.25 (36H, m), 1.63 (14H, m), 2.03 (2H, q, J=7.18 Hz), 2.24 (2H, t, J=7.91 Hz), 2.30 (2H, t, J=7.29 Hz), 2.37 (2H, q, J=7.30 Hz), 2.62 (6H, m), 3.20 (2H, m), 4.06 (2H, t, J=7.08 Hz), 4.16 (2H, t, J=6.65 Hz), 4.35 (1H, dd, J=11.80, 2.56 Hz), 4.44 (1H, dd, J=11.80, 4.41 Hz), 4.79 (1H, m), 5.35 (2H, m), 5.50 (1H, m), 6.44 (1H, d, J=7.77 Hz).

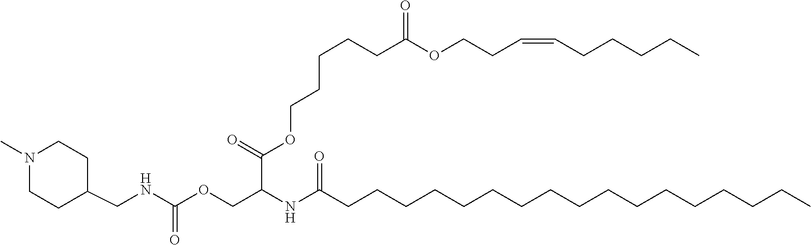 [SMILES: CCCCC/C=C\CCOC(=O)CCCCCOC(=O)C(COC(=O)NCC1CCN(C)CC1)NC(=O)CCCCCCCCCCCCCCCCC]

(Z)-non-3-en-1-yl 6-((O-(((1-methylpiperidin-4-yl)methyl)carbamoyl)-N-stearoylseryl)oxy)hexanoate was synthesized according to representative synthetic scheme 1 and the corresponding methods. MS [ESI]: m/z: [M+H]. calc. 764.6 obs. 764.9. ¹H NMR (400 MHz, CDCl₃): δ 0.87 (6H, m), 1.25 (36H, m), 1.67 (11H, m), 1.91 (2H, t, J=11.78 Hz), 2.03 (2H, q, J=7.23 Hz), 2.23 (2H, t, J=7.81 Hz), 2.27 (3H, s), 2.31 (2H, t, J=7.50 Hz), 2.37 (2H, q, J=7.26 Hz), 2.86 (2H, d, J=11.80 Hz), 3.06 (2H, t, J=6.23 Hz), 4.06 (2H, t, J=7.00 Hz), 4.17 (2H, m), 4.39 (2H, m), 4.78 (1H, m), 4.99 (1H, t, J=5.99 Hz), 5.33 (1H, m), 5.50 (1H, m), 6.40 (1H, d, J=7.72 Hz).

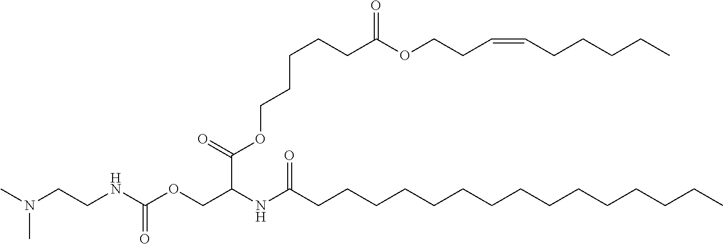 [SMILES: CCCCC/C=C\CCOC(=O)CCCCCOC(=O)C(COC(=O)NCCN(C)C)NC(=O)CCCCCCCCCCCCCCC]

(Z)-non-3-en-1-yl 6-((O-((2-(dimethylamino)ethyl)carbamoyl)-N-palmitoylseryl)oxy)hexanoate was synthesized according to representative synthetic scheme 1 and the corresponding methods. MS [ESI]: m/z: [M+H]. calc. 696.6 obs. 697.1. ¹H NMR (400 MHz, CDCl₃): δ 0.88 (6H, t, J=6.58 Hz), 1.25 (32H, m), 1.65 (6H, m), 2.03 (2H, m), 2.22 (14H, m), 3.24 (2H, m), 4.06 (2H, t, J=6.96 Hz), 4.16 (2H, td, J=6.83, 1.81 Hz), 4.31-4.48 (2H, m), 4.79 (1H, m), 5.30 (2H, m), 5.49 (1H, m), 6.41 (1H, d, J=7.52 Hz).

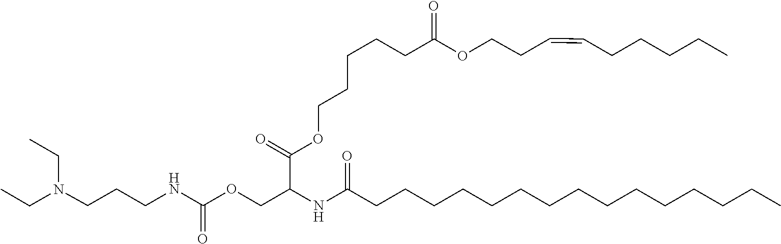 [SMILES: CCCCC/C=C\CCOC(=O)CCCCCOC(=O)C(COC(=O)NCCCN(CC)CC)NC(=O)CCCCCCCCCCCCCCC]

(Z)-non-3-en-1-yl 6-((O-((3-(diethylamino)propyl)carbamoyl)-N-palmitoylseryl)oxy)hexanoate was synthesized according to representative synthetic scheme 1 and the corresponding methods. MS [ESI]: m/z: [M+H]. calc. 738.6 obs. 738.9; ¹H NMR (400 MHz, CDCl₃): δ 0.88 (6H, m), 1.01 (6H, t, J=7.13 Hz), 1.25 (32H, m), 1.63 (8H, m), 2.03 (2H, q, J=6.60 Hz), 2.22 (211, dd, J=9.79, 7.93 Hz), 2.30 (411, m), 2.48 (6H, m), 3.23 (2H, m), 4.06 (2H, t, J=6.92 Hz), 4.11-4.17 (2H, m), 4.29-4.46 (2H, m), 4.75 (1H, m), 5.29 (2H, m), 5.48 (1H, m), 6.36-6.50 (1H, m).

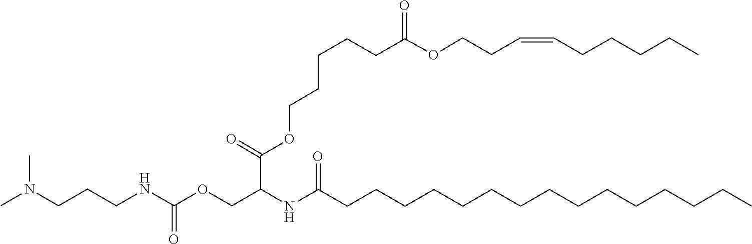 [SMILES: CCCCC/C=C\CCOC(=O)CCCCCOC(=O)C(COC(=O)NCCCN(C)C)NC(=O)CCCCCCCCCCCCCCC]

(Z)-non-3-en-1-yl 6-((O-((3-(dimethylamino)propyl)carbamoyl)-N-palmitoylseryl)oxy)hexanoate was synthesized according to representative synthetic scheme 1 and the corresponding methods. MS [ESI]: m/z: [M+H]. calc. 710.6 obs. 710.9; ¹H NMR (400 MHz, CDCl₃): δ 0.88 (6H, m), 1.25 (32H, m), 1.64 (8H, m), 2.03 (2H, q, J=7.29 Hz), 2.21 (8H, m), 2.33 (6H, m), 3.23 (2H, m), 4.06 (2H, t, J=6.96 Hz), 4.15 (2H, m), 4.41 (2H, m), 4.72-4.82 (1H, m), 5.41 (2H, m), 5.79 (1H, m), 6.46 (1H, d, J=6.48 Hz).

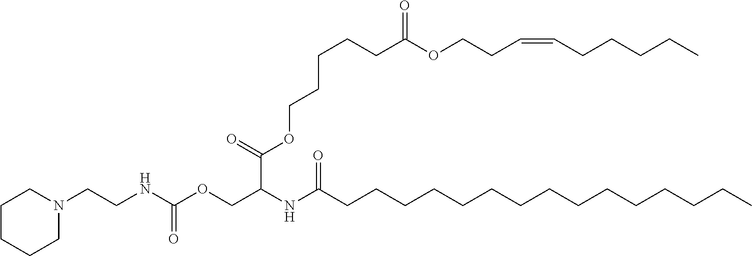 [SMILES: CCCCC/C=C\CCOC(=O)CCCCCOC(=O)C(COC(=O)NCCN1CCCCC1)NC(=O)CCCCCCCCCCCCCCC]

(Z)-non-3-en-1-yl 6-((N-palmitoyl-O-((2-(piperidin-1-yl)ethyl)carbamoyl)seryl)oxy)hexanoate was synthesized according to representative synthetic scheme 1 and the corresponding methods. MS [ESI]: m/z: [M+H]. calc. 736.6 obs. 736.7; ¹H NMR (400 MHz, CDCl₃): δ 0.88 (6H, m), 1.25 (32H, m), 1.65 (12H, m), 2.04 (2H, m), 2.36 (12H, m), 3.24 (2H, m), 4.06 (4H, m), 4.39 (2H, m), 4.79 (1H, m), 5.30 (2H, m), 5.49 (1H, m), 6.44 (1H, d, J=7.86 Hz).

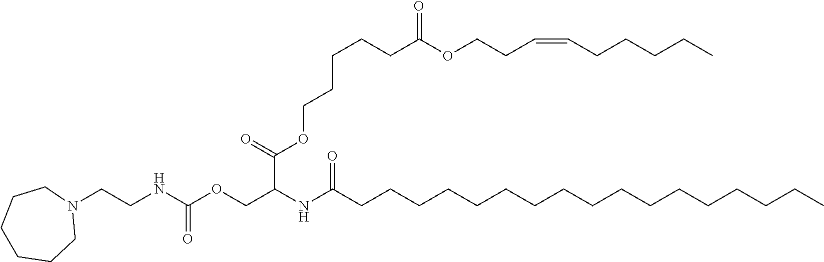 [SMILES: CCCCC/C=C\CCOC(=O)CCCCCOC(=O)C(COC(=O)NCCN1CCCCCC1)NC(=O)CCCCCCCCCCCCCCCCC]

(Z)-non-3-en-1-yl 6-((O-((2-(azepan-1-yl)ethyl)carbamoyl)-N-stearoylseryl)oxy)hexanoate was synthesized according to representative synthetic scheme 1 and the corresponding methods. MS [ESI]: m/z: [M+H]. calc. 778.6 obs. 779.0; ¹H NMR (400 MHz, CDCl₃): δ 0.88 (6H, m), 1.25 (36H, m), 1.65 (14H, m), 2.04 (4H, m), 2.31 (10H, m), 3.44 (2H, m), 4.06 (2H, m), 4.14 (2H, m), 4.21-4.32 (1H, m), 4.44-4.51 (1H, m), 4.79-4.88 (1H, m), 5.26-5.38 (1H, m), 5.44-5.55 (1H, m), 6.90-6.97 (1H, m).

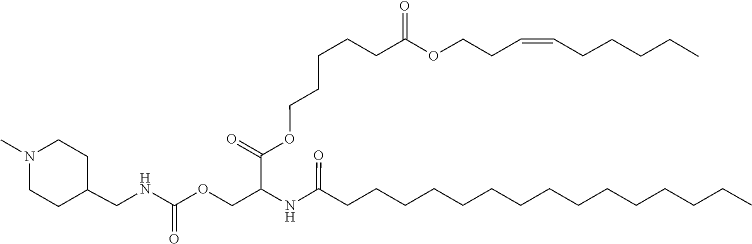 [SMILES: CCCCC/C=C\CCOC(=O)CCCCCOC(=O)C(COC(=O)NCC1CCN(C)CC1)NC(=O)CCCCCCCCCCCCCCC]

(Z)-non-3-en-1-yl 6-((O-(((1-methylpiperidin-4-yl)methyl)carbamoyl)-N-palmitoylseryl)oxy)hexanoate was synthesized according to representative synthetic scheme 1 and the corresponding methods. MS [ESI]: m/z: [M+H]. calc. 736.6 obs. 736.9; ¹H NMR (400 MHz, CDCl₃): δ 0.88 (6H, m), 1.25 (34H, m), 1.65 (9H, m), 1.90 (2H, t, J=11.53 Hz), 2.03 (2H, q, J=7.69 Hz), 2.26 (9H, m), 2.85 (2H, d, J=11.27 Hz), 3.06 (2H, dd, J=3.07, 3.06 Hz), 4.06 (2H, t, J=6.96 Hz), 4.15 (2H, m), 4.38 (2H, m), 4.78 (1H, m), 4.99 (1H, m), 5.41 (2H, m), 6.40 (1H, d, J=7.69 Hz).

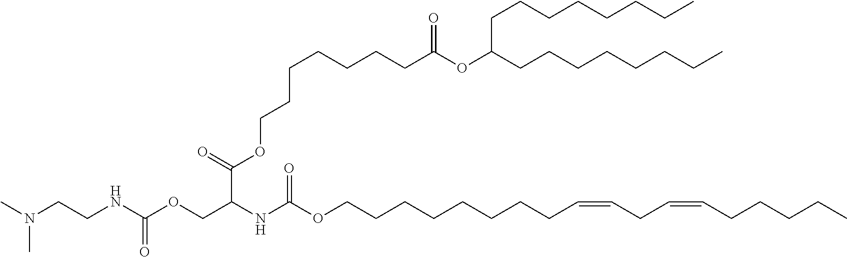 [SMILES: CCCCC/C=C\C/C=C\CCCCCCCCOC(=O)NC(COC(=O)NCCN(C)C)C(=O)OCCCCCCCC(=O)OC(CCCCCCCC)CCCCCCCC]

heptadecan-9-yl 8-((O-((2-(dimethylamino)ethyl)carbamoyl)-N-((((9Z,12Z)-octadeca-9,12-dien-1-yl)oxy)carbonyl)seryl)oxy)octanoate was synthesized according to representative synthetic scheme 2 and the corresponding methods. MS [ESI]: m/z: [M+H]. calc. 892.7 obs. 892.5; ¹H NMR (400 MHz, CDCl₃): δ 0.88 (9H, m), 1.26 (46H, m), 1.44-1.55 (4H, m), 1.62 (6H, m), 2.05 (4H, q, J=6.87 Hz), 2.22 (6H, s), 2.27 (2H, dd, J=7.94, 7.26 Hz), 2.38 (2H, dd, J=7.16, 5.96 Hz), 2.77 (2H, td, J=6.41, 1.37 Hz), 3.23 (2H, m), 4.06 (2H, dd, J=8.00, 7.03 Hz), 4.15 (2H, t, J=6.75 Hz), 4.30-4.48 (2H, m), 4.48-4.59 (1H, m), 4.86 (1H, i, J=6.50 Hz), 5.24 (1H, m), 5.36 (4H, m), 5.53 (1H, d, J=8.37 Hz).

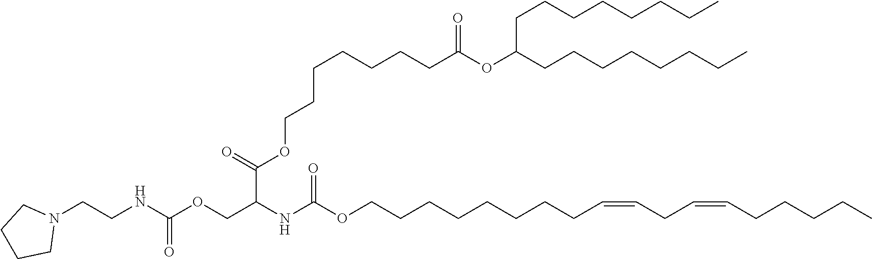 [SMILES: CCCCC/C=C\C/C=C\CCCCCCCCOC(=O)NC(COC(=O)NCCN1CCCC1)C(=O)OCCCCCCCC(=O)OC(CCCCCCCC)CCCCCCCC]

heptadecan-9-yl 8-((N-((((9Z,12Z)-octadeca-9,12-dien-1-yl)oxy)carbonyl)-O-((2-(pyrrolidin-1-yl)ethyl)carbamoyl)seryl)oxy)octanoate was synthesized according to representative synthetic scheme 2 and the corresponding methods. MS [ESI]: m/z: [M+H]. calc. 918.7 obs. 919.2; ¹H NMR (400 MHz, CDCl₃): δ 0.88 (9H, m), 1.26 (46H, m), 1.50 (4H, m), 1.57-1.68 (6H, m), 1.77 (4H, m), 2.05 (4H, q, J=6.89 Hz), 2.27 (2H, dd, J=7.94, 7.09 Hz), 2.50 (4H, m), 2.57 (2H, dd, J=6.69, 6.31 Hz), 2.77 (2H, t, J=6.23 Hz), 3.23-3.33 (2H, m), 4.06 (2H, dd, J=7.91, 6.86 Hz), 4.15 (2H, t, J=6.84 Hz), 4.30-4.48 (2H, m), 4.49-4.59 (1H, m), 4.86 (1H, i, J=6.83 Hz), 5.36 (5H, m), 5.53 (1H, d, J=8.20 Hz).

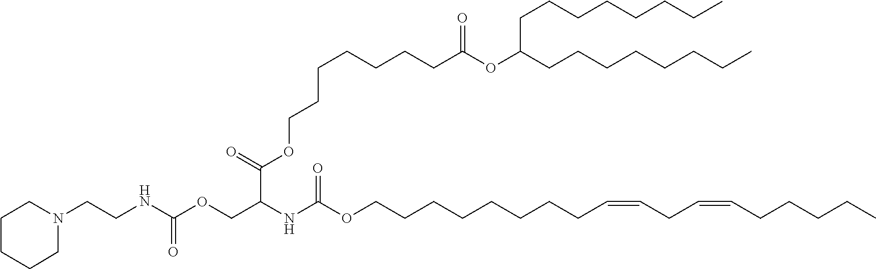 [SMILES: CCCCC/C=C\C/C=C\CCCCCCCCOC(=O)NC(COC(=O)NCCN1CCCCC1)C(=O)OCCCCCCCC(=O)OC(CCCCCCCC)CCCCCCCC]

heptadecan-9-yl 8-((N-((((9Z,12Z)-octadeca-9,12-dien-1-yl)oxy)carbonyl)-O-((2-(piperidin-1-yl)ethyl)carbamoyl)seryl)oxy)octanoate was synthesized according to representative synthetic scheme 2 and the corresponding methods. MS [ESI]: m/z: [M+H]. calc. 932.8 ohs. 933.0; ¹H NMR (400 MHz, CDCl₃): δ 0.87 (9H, m), 1.25 (46H, m), 1.41-1.55 (6H, m), 1.62 (10H, m), 2.05 (4H, q, J=7.00 Hz), 2.27 (2H, dd, J=7.94, 7.09 Hz), 2.35-2.56 (6H, m), 2.77 (2H, dd, J=7.36, 6.68 Hz), 3.29 (2H, m), 4.06 (2H, dd, J=8.38, 6.87 Hz), 4.15 (2H, t, J=6.75 Hz), 4.30-4.48 (2H, m), 4.50-4.59 (1H, m), 4.86 (1H, i, J=6.26 Hz), 5.35 (5H, m), 5.53-5.63 (1H, m).

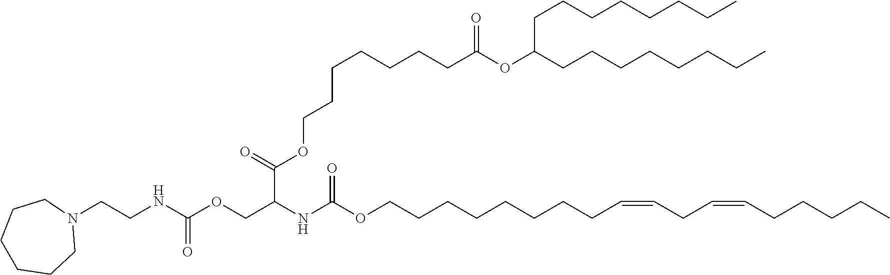 [SMILES: CCCCC/C=C\C/C=C\CCCCCCCCOC(=O)NC(COC(=O)NCCN1CCCCCC1)C(=O)OCCCCCCCC(=O)OC(CCCCCCCC)CCCCCCCC]

heptadecan-9-yl 8-((O-((2-(azepan-1-yl)ethyl)carbamoyl)-N-((((9Z,12Z)-octadeca-9,12-dien-1-yl)oxy)carbonyl)seryl)oxy)octanoate was synthesized according to representative synthetic scheme 2 and the corresponding methods. MS [ESI]: m/z: [M+H]. calc. 946.8 obs. 946.5; ¹H NMR (400 MHz, CDCl₃): δ 0.88 (9H, d, J=5.12 Hz), 1.25 (46H, m), 1.44-1.55 (4H, m), 1.55-1.71 (14H, m), 2.05 (4H, q, J=7.34 Hz), 2.27 (2H, dd, J=7.94, 7.09 Hz), 2.57-2.72 (6H, m), 2.77 (2H, dd, J=7.45, 6.45 Hz), 3.17-3.31 (2H, m), 4.06 (2H, dd, J=8.10, 7.08 Hz), 4.15 (2H, t, J=6.84 Hz), 4.30-4.48 (2H, m), 4.49-4.58 (1H, m), 4.86 (1H, i, J=6.83 Hz), 5.35 (4H, m), 5.52-5.62 (2H, m).

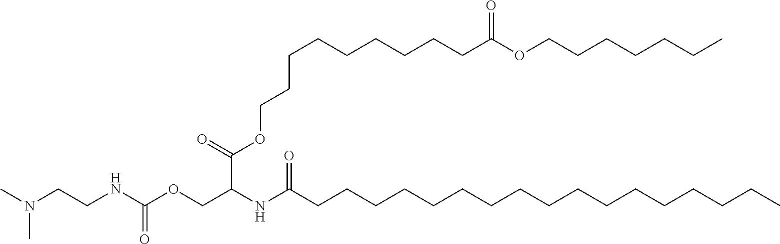 [SMILES: CCCCCCCCCCCCCCCCCC(=O)NC(COC(=O)NCCN(C)C)C(=O)OCCCCCCCCCC(=O)OCCCCCCC]

heptyl 10-((O-((2-(dimethylamino)ethyl)carbamoyl)-N-stearoylseryl)oxy)decanoate octanoate was synthesized according to representative synthetic scheme 1 and the corresponding methods. MS [ESI]: m/z: [M+H]. calc. 753.6 obs. 753.9; ¹H NMR (400 MHz, CDCl₃): δ 0.88 (6H, t, J=6.84 Hz), 1.25 (44H, m), 1.63 (10H, m), 2.22 (8H, m), 2.31 (2H, t, J=7.43 Hz), 2.40 (2H, dd, J=7.14, 5.90 Hz), 3.20-3.28 (2H, m), 4.05 (2H, t, J=6.97 Hz), 4.16 (2H, m), 4.30-4.47 (2H, m), 4.75-4.84 (1H, m), 5.29-5.36 (1H, m), 6.37-6.44 (1H, m).

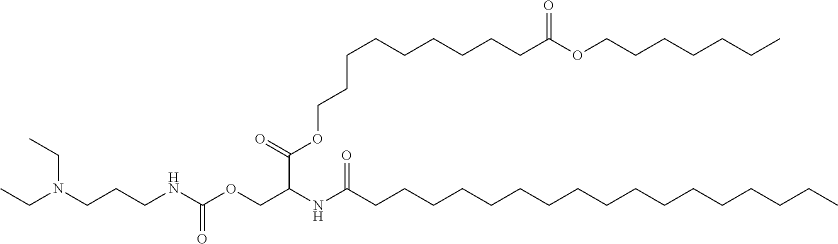 [SMILES: CCCCCCCCCCCCCCCCCC(=O)NC(COC(=O)NCCCN(CC)CC)C(=O)OCCCCCCCCCC(=O)OCCCCCCC]

heptyl 10-((O-((3-(diethylamino)propyl)carbamoyl)-N-stearoylseryl)oxy)decanoate was synthesized according to representative synthetic scheme 1 and the corresponding methods. MS [ESI]: m/z: [M+H]. calc. 796.7 obs. 796.9; ¹H NMR (400 MHz, CDCl₃): δ 0.88 (6H, m), 1.08-1.19 (6H, m), 1.25 (42H, m), 1.55-1.72 (14H, m), 2.24 (2H, dd, J=8.33, 6.95 Hz), 2.31 (2H, t, J=7.45 Hz), 2.59-2.76 (6H, m), 3.24-3.32 (2H, m), 4.05 (2H, t, J=6.79 Hz), 4.12-4.18 (2H, m), 4.28-4.35 (1H, m), 4.40-4.47 (1H, m), 4.74-4.82 (1H, m), 6.24-6.34 (1H, m), 6.48-6.56 (1H, m).

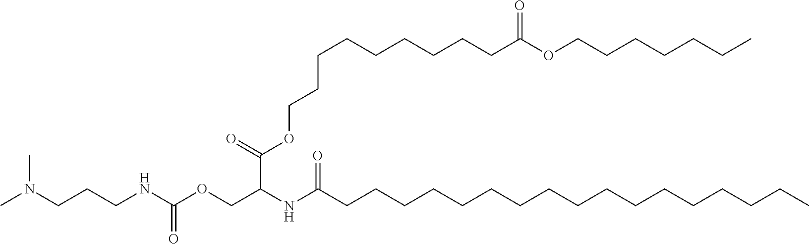 [SMILES: CCCCCCCCCCCCCCCCCC(=O)NC(COC(=O)NCCCN(C)C)C(=O)OCCCCCCCCCC(=O)OCCCCCCC]

heptyl 10-((O-((3-(dimethylamino)propyl)carbamoyl)-N-stearoylseryl)oxy)decanoate was synthesized according to representative synthetic scheme 1 and the corresponding methods. MS [ESI]: m/z: [M+H]. calc. 768.6 obs. 768.3; ¹H NMR (400 MHz, CDCl₃): δ 0.88 (6H, t, J=6.89 Hz), 1.25 (44H, m), 1.57-1.72 (12H, m), 2.26 (2H, dd, J=8.33, 6.84 Hz), 2.31 (2H, dd, J=7.69, 7.05 Hz), 2.48 (6H, s), 2.63-2.71 (2H, m), 3.24-3.32 (2H, m), 4.05 (2H, m), 4.12-4.19 (2H, m), 4.30-4.47 (2H, m), 4.75-4.82 (1H, m), 5.88-5.93 (1H, m), 6.53-6.59 (1H, m).

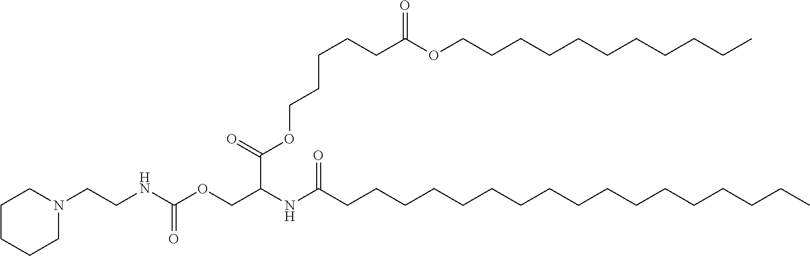 [SMILES: CCCCCCCCCCCCCCCCCC(=O)NC(COC(=O)NCCN1CCCCC1)C(=O)OCCCCCC(=O)OCCCCCCCCCCC]

undecyl 6-((O-((2-(piperidin-1-yl)ethyl)carbamoyl)-N-stearoylseryl)oxy)hexanoate was synthesized according to representative synthetic scheme 1 and the corresponding methods. MS [ESI]: m/z: [M+H]. calc. 794.7 obs. 794.3; ¹H NMR (400 MHz, CDCl₃): δ 0.88 (6H, m), 1.25 (44H, m), 1.36-1.43 (2H, m), 1.57 (6H, m), 1.59-1.71 (8H, m), 2.24 (2H, dd, J=9.32, 8.08 Hz), 2.31 (2H, t, J=7.56 Hz), 2.33-2.45 (6H, m), 3.21-3.30 (2H, m), 4.05 (2H, t, J=6.79 Hz), 4.14-4.19 (2H, m), 4.31-4.37 (1H, m), 4.42-4.47 (1H, m), 4.77-4.84 (2H, m), 5.29-5.36 (1H, m), 6.40-6.47 (1H, m).

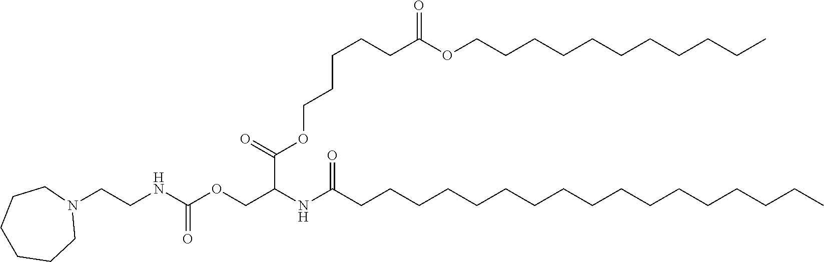 [SMILES: CCCCCCCCCCCCCCCCCC(=O)NC(COC(=O)NCCN1CCCCCC1)C(=O)OCCCCCC(=O)OCCCCCCCCCCC]

undecyl 6-((O-((2-(azepan-1-yl)ethyl)carbamoyl)-N-stearoylseryl)oxy)hexanoate was synthesized according to representative synthetic scheme 1 and the corresponding methods. MS [ESI]: m/z: [M+H]. calc. 808.7 obs. 809.1; ¹H NMR (400 MHz, CDCl₃): δ 0.88 (6H, t, J=6.89 Hz), 1.25 (44H, m), 1.56-1.71 (18H, m), 2.31 (5H, m), 2.70-2.90 (5H, m), 3.29-3.36 (2H, m), 4.05 (2H, t, J=6.79 Hz), 4.15 (2H, t, J=6.83 Hz), 4.26-4.54 (2H, m), 4.76-4.85 (1H, m), 6.52-6.93 (2H, m).

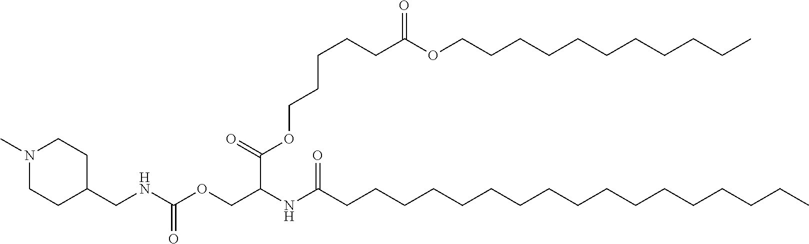 [SMILES: CCCCCCCCCCCCCCCCCC(=O)NC(COC(=O)NCC1CCN(C)CC1)C(=O)OCCCCCC(=O)OCCCCCCCCCCC]

undecyl 6-((O-(((1-methylpiperidin-4-yl)methyl)carbamoyl)-N-stearoylseryl)oxy)hexanoate was synthesized according to representative synthetic scheme 1 and the corresponding methods. MS [ESI]: m/z: [M+H]. calc. 794.7 obs. 794.9; ¹H NMR (400 MHz, CDCl₃): δ 0.88 (6H, m), 1.25 (46H, m), 1.39 (1H, m), 1.57-1.72 (12H, m), 1.91 (2H, dd, J=13.32, 12.16 Hz), 2.22 (2H, t, J=7.95 Hz), 2.27 (3H, s), 2.31 (2H, dd, J=7.69, 7.05 Hz), 2.82-2.89 (2H, m), 3.06 (2H, t, J=6.25 Hz), 4.05 (2H, t, J=6.79 Hz), 4.11-4.22 (2H, m), 4.32-4.44 (2H, m), 4.75-4.81 (1H, m), 5.30 (1H, s), 6.37-6.43 (1H, m).

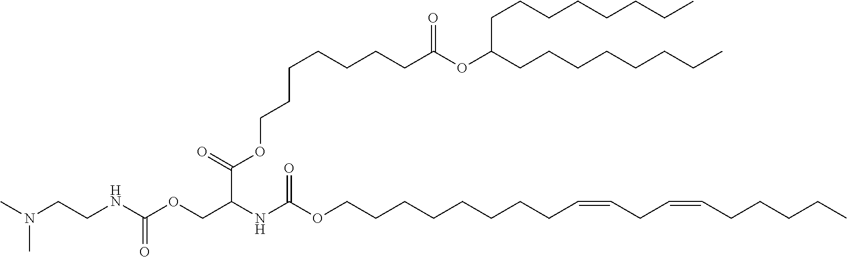 [SMILES: CCCCC/C=C\C/C=C\CCCCCCCCOC(=O)NC(COC(=O)NCCN(C)C)C(=O)OCCCCCCCC(=O)OC(CCCCCCCC)CCCCCCCC]

heptadecan-9-yl 8-((O-((2-(dimethylamino)ethyl)carbamoyl)-N-((((9Z,12Z)-octadeca-9,12-dien-1-yl)oxy)carbonyl)seryl)oxy)octanoate was synthesized according to representative synthetic scheme 2 and the corresponding methods. MS [ESI]: m/z: [M+H]. calc. 892.7 obs. 892.9; ¹H NMR (400 MHz, CDCl₃): δ 0.88 (9H, m), 1.25 (46H, m), 1.44-1.54 (4H, m), 1.55-1.71 (6H, m), 2.05 (4H, q, J=7.26 Hz), 2.30 (8H, m), 2.49 (2H, m), 2.77 (2H, dd, J=7.47, 6.60 Hz), 3.28 (2H, m), 4.06 (2H, dd, J=8.55, 7.05 Hz), 4.15 (2H, t, J=6.75 Hz), 4.30-4.47 (2H, m), 4.49-4.58 (1H, m), 4.86 (1H, i, J=6.32 Hz), 5.35 (4H, m), 5.42-5.49 (1H, m), 5.54-5.64 (1H, m).

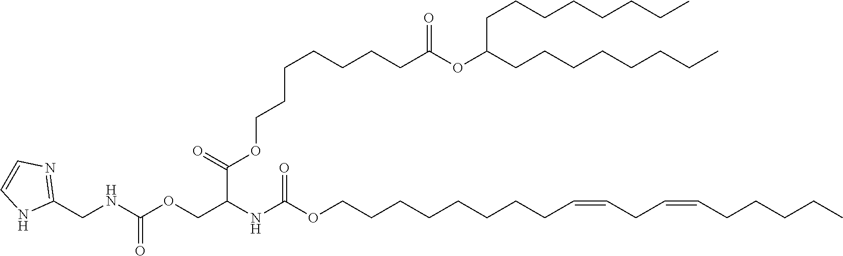 [SMILES: CCCCC/C=C\C/C=C\CCCCCCCCOC(=O)NC(COC(=O)NCc1ncc[nH]1)C(=O)OCCCCCCCC(=O)OC(CCCCCCCC)CCCCCCCC]

heptadecan-9-yl 8-((O-(((1H-imidazol-2-yl)methyl)carbamoyl)-N-((((9Z,12Z)-octadeca-9,12-dien-1-yl)oxy)carbonyl)seryl)oxy)octanoate was synthesized according to representative synthetic scheme 2 and the corresponding methods. MS [ESI]: m/z: [M+H]. calc. 901.7 obs. 901.8; ¹H NMR (400 MHz, CDCl₃): δ 0.87 (9H, m), 1.25 (46H, m), 1.50 (4H, m), 1.55-1.67 (6H, m), 2.05 (4H, m), 2.28 (2H, dd, J=7.77, 7.09 Hz), 2.77 (2H, dd, J=7.43, 6.37 Hz), 3.99-4.11 (2H, m), 4.15 (2H, dd, J=7.82, 6.72 Hz), 4.29-4.41 (2H, m), 4.41-4.51 (2H, m), 4.59-4.68 (1H, m), 4.86 (1H, i, J=6.49 Hz), 5.35 (4H, m), 5.48-5.61 (1H, m), 6.97 (2H, s), 8.02 (1H, s).

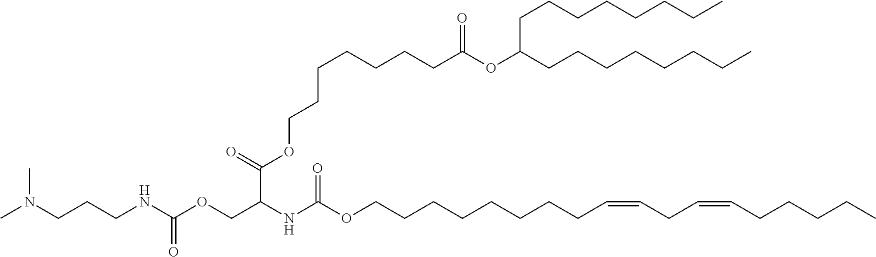 [SMILES: CCCCC/C=C\C/C=C\CCCCCCCCOC(=O)NC(COC(=O)NCCCN(C)C)C(=O)OCCCCCCCC(=O)OC(CCCCCCCC)CCCCCCCC]

heptadecan-9-yl 8-((O-((3-(dimethylamino)propyl)carbamoyl)-N-((((9Z,12Z)-octadeca-9,12-dien-1-yl)oxy)carbonyl)seryl)oxy)octanoate was synthesized according to representative synthetic scheme 2 and the corresponding methods. MS [ESI]: m/z: [M+H]. calc. 906.7 obs. 906.5; ¹H NMR (400 MHz, CDCl₃): δ 0.88 (9H, m), 1.26 (44H, m), 1.50 (4H, m), 1.55-1.70 (10H, m), 2.05 (4H, q, J=7.14 Hz), 2.21 (6H, s), 2.33 (3H, m), 2.61 (1H, s), 2.77 (2H, dd, J=7.40, 6.39 Hz), 3.23 (2H, m), 4.06 (2H, dd, J=7.89, 6.89 Hz), 4.14 (2H, t, J=6.77 Hz), 4.28-4.47 (2H, m), 4.49-4.58 (1H, m), 4.86 (1H, i, J=6.66 Hz), 5.36 (4H, m), 5.54 (1H, d, J=8.29 Hz), 5.64-5.72 (1H, m).

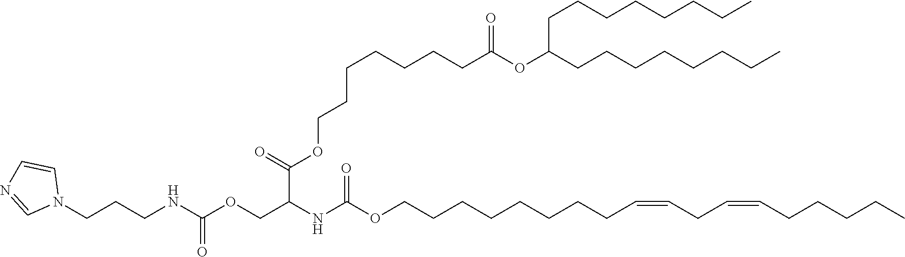 [SMILES: CCCCC/C=C\C/C=C\CCCCCCCCOC(=O)NC(COC(=O)NCCCn1ccnc1)C(=O)OCCCCCCCC(=O)OC(CCCCCCCC)CCCCCCCC]

heptadecan-9-yl 8-((O-((3-(1H-imidazol-1-yl)propyl)carbamoyl)-N-((((9Z,12Z)-octadeca-9,12-dien-1-yl)oxy)carbonyl)seryl)oxy)octanoate was synthesized according to representative synthetic scheme 2 and the corresponding methods. MS [ESI]: m/z: [M+H]. calc. 929.7 obs. 930.1; ¹H NMR (400 MHz, CDCl₃): δ 0.87 (9H, m), 1.25 (46H, m), 1.44-1.55 (4H, m), 1.55-1.70 (6H, m), 2.05 (6H, m), 2.27 (2H, dd, J=7.86, 7.09 Hz), 2.77 (2H, dd, J=7.29, 6.55 Hz), 3.19 (2H, m), 4.06 (2H, m), 4.15 (4H, m), 4.30-4.46 (2H, m), 4.50-4.60 (1H, m), 4.85 (1H, i, J=6.49 Hz), 5.35 (4H, m), 5.46-5.56 (1H, m), 5.66-5.75 (1H, m), 7.09 (1H, m), 7.22 (1H, m), 8.57 (1H, br s).

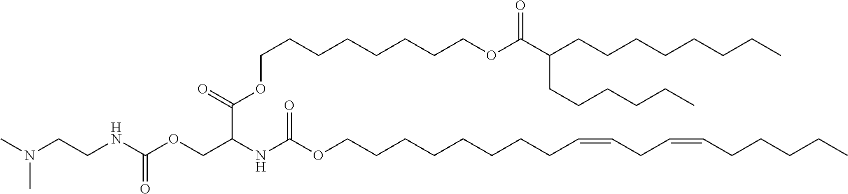 [SMILES: CCCCC/C=C\C/C=C\CCCCCCCCOC(=O)NC(COC(=O)NCCN(C)C)C(=O)OCCCCCCCCOC(=O)C(CCCCCC)CCCCCCCC]

2-methyl-9-(((((9Z,12Z)-octadeca-9,12-dien-1-yl)oxy)carbonyl)amino)-6,10-dioxo-7,11-dioxa-2,5-diazanonadecan-19-yl 2-hexyldecanoate was synthesized according to representative synthetic scheme 2 and the corresponding methods. MS [ESI]: m/z: [M+H]. calc. 878.7 obs. 878.3; ¹H NMR (400 MHz, CDCl₃): δ 0.88 (9H, m), 1.25 (46H, m), 1.61 (8H, m), 2.04 (5H, m), 2.29 (7H, m), 2.61 (1H, m), 2.77 (2H, dd, J=6.88, 6.13 Hz), 3.20-3.34 (2H, m), 4.06 (4H, m), 4.15 (2H, m), 4.29-4.48 (2H, m), 4.50-4.59 (1H, m), 5.36 (5H, m), 5.58 (1H, d, J=7.32 Hz).

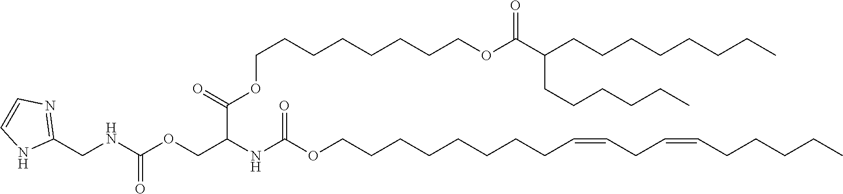 [SMILES: CCCCC/C=C\C/C=C\CCCCCCCCOC(=O)NC(COC(=O)NCc1ncc[nH]1)C(=O)OCCCCCCCCOC(=O)C(CCCCCC)CCCCCCCC]

8-((O-(((1H-imidazol-2-yl)methyl)carbamoyl)-N-((((9Z,12Z)-octadeca-9,12-dien-1-yl)oxy)carbonyl)seryl)oxy)octyl 2-hexyldecanoate was synthesized according to representative synthetic scheme 2 and the corresponding methods. MS [ESI]: m/z: [M+H]. calc. 887.7 obs. 888.2; ¹H NMR (400 MHz, CDCl₃): δ 0.87 (9H, m), 1.25 (46H, m), 1.52-1.69 (8H, m), 2.04 (4H, m), 2.25-2.36 (1H, m), 2.77 (2H, m), 4.06 (6H, m), 4.38-4.52 (4H, m), 4.58-4.73 (1H, m), 5.30 (5H, m), 5.51-5.61 (1H, m), 5.77-5.89 (1H, m), 7.01 (2H, m).

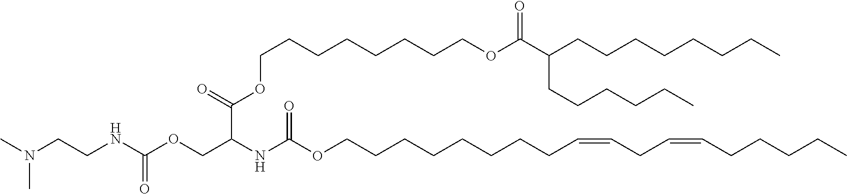 [SMILES: CCCCC/C=C\C/C=C\CCCCCCCCOC(=O)NC(COC(=O)NCCN(C)C)C(=O)OCCCCCCCCOC(=O)C(CCCCCC)CCCCCCCC]

2-methyl-10-(((((9Z,12Z)-octadeca-9,12-dien-1-yl)oxy)carbonyl)amino)-7,11-dioxo-8,12-dioxa-2,6-diazaicosan-20-yl 2-hexyldecanoate was synthesized according to method. MS [ESI]: m/z: [M+H]. calc. 892.7 obs. 892.8; ¹H NMR (400 MHz, CDCl₃): δ 0.88 (9H, m), 1.25 (44H, m), 1.63 (12H, m), 2.05 (4H, m), 2.21 (6H, s), 2.33 (3H, m), 2.77 (2H, dd, J=7.16, 6.23 Hz), 3.23 (2H, m), 4.06 (4H, t, J=6.70 Hz), 4.15 (2H, m), 4.27-4.49 (2H, m), 4.50-4.60 (1H, m), 5.30 (5H, m), 5.49-5.58 (1H, m).

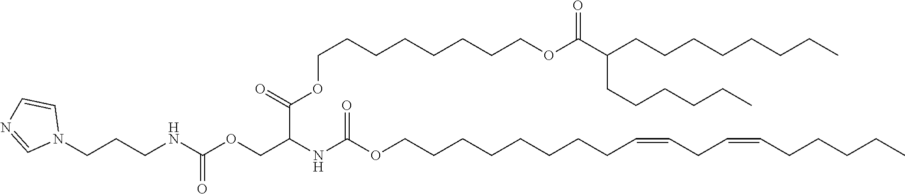 [SMILES: CCCCC/C=C\C/C=C\CCCCCCCCOC(=O)NC(COC(=O)NCCCn1ccnc1)C(=O)OCCCCCCCCOC(=O)C(CCCCCC)CCCCCCCC]

8-((O-((3-(1H-imidazol-1-yl)propyl)carbamoyl)-N-((((9Z,12Z)-octadeca-9,12-dien-1-yl)oxy)carbonyl)seryl)oxy)octyl 2-hexyldecanoate was synthesized according to representative synthetic scheme 2 and the corresponding methods. MS [ESI]: m/z: [M+H]. calc. 915.7 obs. 915.9; ¹H NMR (400 MHz, CDCl₃): δ 0.88 (9H, m), 1.25 (46H, m), 1.60 (8H, m), 1.94 (6H, m), 2.30 (1H, m), 2.77 (2H, dd, J=7.41, 6.49 Hz), 3.13-3.23 (2H, m), 4.06 (7H, m), 4.15 (2H, m), 4.39 (2H, m), 4.51-4.61 (1H, m), 4.96-5.07 (1H, m), 5.35 (5H, m), 5.58 (1H, m), 6.99 (1H, m), 7.12 (1H, m), 7.81 (1H, s).

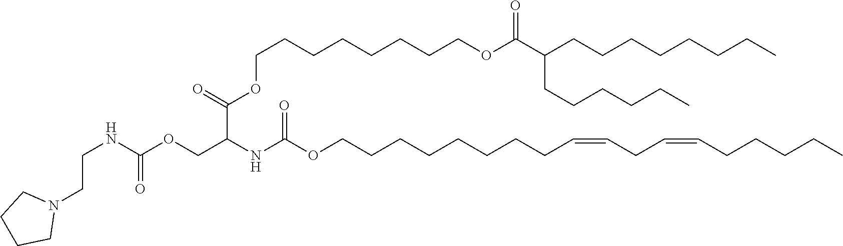 [SMILES: CCCCC/C=C\C/C=C\CCCCCCCCOC(=O)NC(COC(=O)NCCN1CCCC1)C(=O)OCCCCCCCCOC(=O)C(CCCCCC)CCCCCCCC]

8-((N-((((9Z,12Z)-octadeca-9,12-dien-1-yl)oxy)carbonyl)-O-((2-(pyrrolidin-1-yl)ethyl)carbamoyl)seryl)oxy)octyl 2-hexyldecanoate was synthesized according to representative synthetic scheme 2 and the corresponding methods. MS [ESI]: m/z: [M+H]. calc. 904.7 obs. 905.1; 1H NMR (400 MHz, CDCl₃): δ 0.87 (9H, m), 1.25 (44H, m), 1.60 (8H, m), 1.79-1.91 (6H, m), 2.05 (5H, m), 2.30 (1H, m), 2.61-2.75 (5H, m), 2.75-2.80 (2H, m), 3.33 (2H, m), 4.06 (4H, m), 4.15 (2H, dd, J=7.57, 6.81 Hz), 4.31-4.47 (2H, m), 4.51-4.59 (1H, m), 5.35 (4H, m), 5.50-5.71 (2H, s).

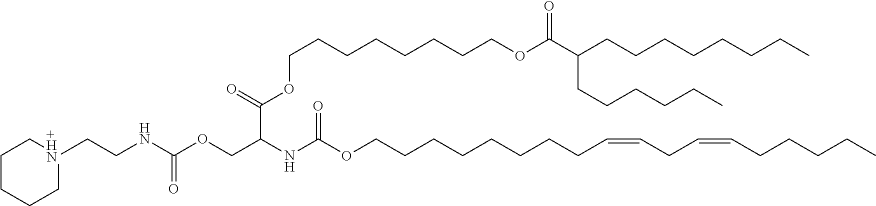

8-((N-((((9Z,12Z)-octadeca-9,12-dien-1-yl)oxy)carbonyl)-O-((2-(piperidin-1-yl)ethyl)carbamoyl)seryl)oxy)octyl 2-hexyldecanoate was synthesized according to representative synthetic scheme 2 and the corresponding methods. MS [ESI]: m/z: [M+H]. calc. 918.7 obs. 918.8; ¹H NMR (400 MHz, CDCl₃): δ 0.88 (9H, m), 1.25 (46H, m), 1.51-1.71 (14H, m), 2.05 (5H, m), 2.40-2.61 (6H, m), 2.77 (2H, dd, J=7.42, 6.39 Hz), 3.25-3.36 (2H, m), 4.06 (6H, m), 4.30-4.39 (1H, m), 4.40-4.48 (1H, m), 4.51-4.59 (1H, m), 5.35 (5H, m), 5.55-5.68 (1H, m).

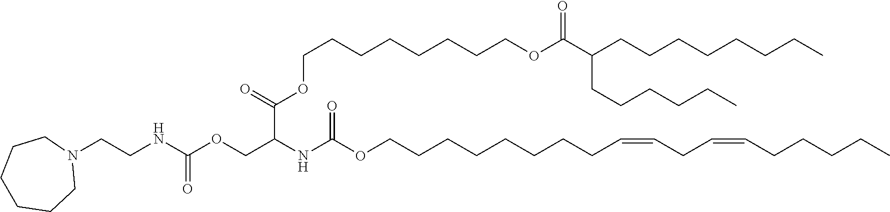 [SMILES: CCCCC/C=C\C/C=C\CCCCCCCCOC(=O)NC(COC(=O)NCCN1CCCCCC1)C(=O)OCCCCCCCCOC(=O)C(CCCCCC)CCCCCCCC]

8-((O-((2-(azepan-1-yl)ethyl)carbamoyl)-N-((((9Z,12Z)-octadeca-9,12-dien-1-yl)oxy)carbonyl)seryl)oxy)octyl 2-hexyldecanoate was synthesized according to representative synthetic scheme 2 and the corresponding methods. MS [ESI]: m/z: [M+H]. calc. 932.8 obs. 933.2; ¹H NMR (400 MHz, CDCl₃): δ 0.87 (9H, t, J=6.53 Hz), 1.25 (46H, m), 1.53-1.72 (16H, m), 2.04 (5H, m), 2.30 (1H, m), 2.58-2.80 (7H, m), 3.26 (2H, m), 4.06 (6H, m), 4.30-4.47 (2H, m), 4.54 (1H, m), 5.35 (4H, m), 5.51-5.64 (1H, m).

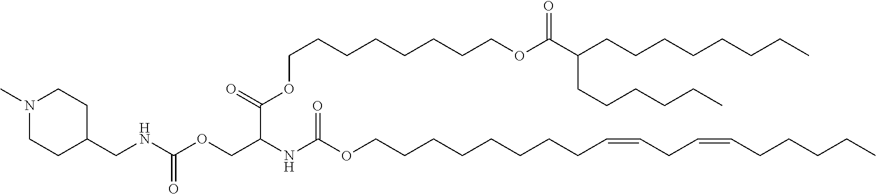 [SMILES: CCCCC/C=C\C/C=C\CCCCCCCCOC(=O)NC(COC(=O)NCC1CCN(C)CC1)C(=O)OCCCCCCCCOC(=O)C(CCCCCC)CCCCCCCC]

8-((O-(((1-methylpiperidin-4-yl)methyl)carbamoyl)-N-((((9Z,12Z)-octadeca-9,12-dien-1-yl)oxy)carbonyl)seryl)oxy)octyl 2-hexyldecanoate was synthesized according to representative synthetic scheme 2 and the corresponding methods. MS [ESI]: m/z: [M+H]. calc. 918.7 obs. 919.2; ¹H NMR (400 MHz, CDCl₃): δ 0.88 (9H, m), 1.25 (45H, m), 1.53-1.76 (12H, m), 2.04 (6H, m), 2.30 (4H, m), 2.61 (2H, s), 2.77 (2H, dd, J=7.49, 6.36 Hz), 2.84-2.93 (2H, m), 3.06 (2H, m), 4.06 (4H, m), 4.14 (2H, m), 4.29-4.45 (2H, m), 4.49-4.58 (1H, m), 5.29 (5H, m), 5.45-5.53 (1H, m).

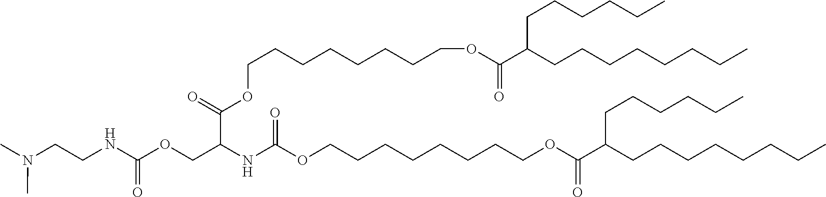

11-((((2-(dimethylamino)ethyl)carbamoyl)oxy)methyl)-25-hexyl-10,13,24-trioxo-9,14,23-trioxa-12-azatritriacontyl 2-hexyldecanoate was synthesized according to representative synthetic scheme 2 and the corresponding methods. MS [ESI]: m/z: [M+H]. calc. 996.8 obs. 996.9; ¹H NMR (400 MHz, CDCl₃): δ 0.87 (12H, t, J=6.49 Hz), 1.25 (60H, m), 1.61 (12H, m), 2.33 (8H, m), 2.52 (2H, m), 3.29 (2H, m), 4.06 (6H, t, J=6.66 Hz), 4.15 (2H, dd, J=7.50, 6.85 Hz), 4.30-4.49 (2H, m), 4.50-4.59 (1H, m), 5.40-5.56 (1H, m), 5.61 (1H, d, J=7.86 Hz).

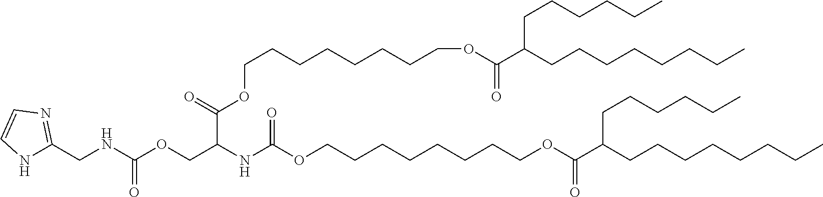

11-(((((1H-imidazol-2-yl)methyl)carbamoyl)oxy)methyl)-25-hexyl-10,13,24-trioxo-9,14,23-trioxa-12-azatritriacontyl 2-hexyldecanoate was synthesized according to representative synthetic scheme 2 and the corresponding methods. MS [ESI]: m/z: [M+H]. calc. 1005.8 obs. 1006.1; ¹H NMR (400 MHz, CDCl₃): δ 0.87 (12H, t, J=6.58 Hz), 1.25 (60H, m), 1.61 (12H, s), 2.31 (2H, m), 4.06 (6H, td, J=6.75, 1.54 Hz), 4.11-4.19 (2H, m), 4.23-4.35 (1H, m), 4.43-4.61 (3H, m), 4.61-4.72 (1H, m), 5.58 (1H, d, J=8.11 Hz), 5.93 (1H, m), 7.03 (2H, m).

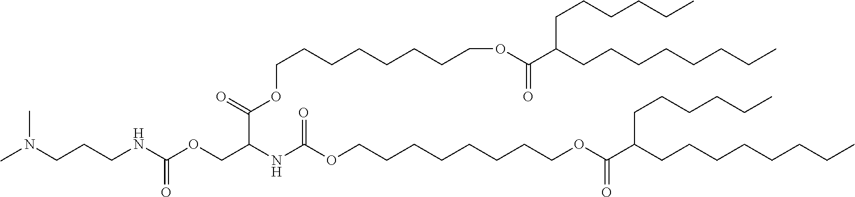

11-((((3-(dimethylamino)propyl)carbamoyl)oxy)methyl)-25-hexyl-10,13,24-trioxo-9,14,23-trioxa-12-azatritriacontyl 2-hexyldecanoate was synthesized according to representative synthetic scheme 2 and the corresponding methods. MS [ESI]: m/z: [M+H]. calc. 1010.8 obs. 1010.3; ¹H NMR (400 MHz, CDCl₃): δ 0.87 (12H, t, J=6.58 Hz), 1.25 (74H, m), 2.33 (8H, m), 2.48 (2H, m), 3.22-3.30 (2H, m), 4.06 (6H, t, J=6.49 Hz), 4.14 (2H, m), 4.27-4.60 (3H, m), 5.71 (2H, m).

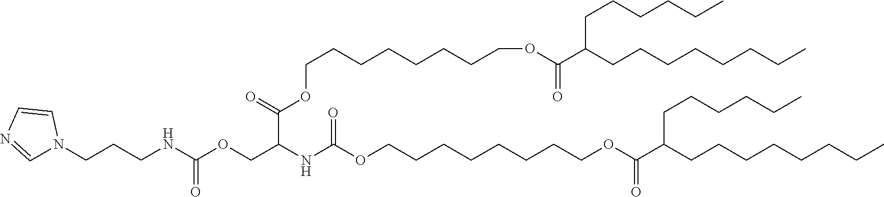

11-((((3-(1H-imidazol-1-yl)propyl)carbamoyl)oxy)methyl)-25-hexyl-10,13,24-trioxo-9,14,23-trioxa-12-azatritriacontyl 2-hexyldecanoate hexyldecanoate was synthesized according to representative synthetic scheme 2 and the corresponding methods. MS [ESI]: m/z: [M+H]. calc. 1033.8 obs. 1034.0; ¹H NMR (400 MHz, CDCl₃): δ 0.87 (12H, t, J=6.58 Hz), 1.25 (60H, m), 1.61 (12H, m), 2.30 (2H, m), 2.30 (2H, m), 3.19 (2H, m), 4.06 (8H, m), 4.14 (2H, m), 4.39 (2H, m), 4.56 (1H, m), 5.01 (1H, m), 5.59 (1H, d, J=8.03 Hz), 6.98 (1H, m), 7.11 (1H, m), 7.76 (1H, br s).

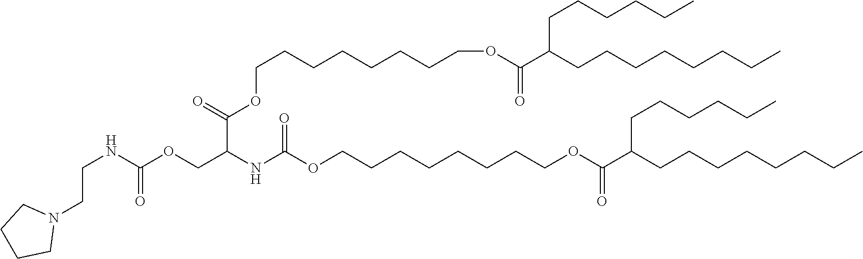 [SMILES: CCCCCCCCC(CCCCCC)C(=O)OCCCCCCCCOC(=O)NC(COC(=O)NCCN1CCCC1)C(=O)OCCCCCCCCOC(=O)C(CCCCCC)CCCCCCCC]

25-hexyl-10,13,24-trioxo-11-((((2-(pyrrolidin-1-yl)ethyl)carbamoyl)oxy)methyl)-9,14,23-trioxa-12-azatritriacontyl 2-hexyldecanoate was synthesized according to representative synthetic scheme 2 and the corresponding methods. MS [ESI]: m/z: [M+H]. calc. 1022.8 obs. 1022.9; ¹H NMR (400 MHz, CDCl₃): δ 0.87 (12H, m), 1.27 (58H, m), 1.61 (12H, m), 1.86 (6H, m), 2.30 (2H, m), 2.71 (6H, m), 3.34 (2H, m), 4.06 (6H, t, J=6.62 Hz), 4.15 (2H, dd, J=7.58, 6.74 Hz), 4.31-4.39 (1H, m), 4.40-4.48 (1H, m), 4.49-4.59 (1H, m), 5.57-5.70 (1H, m).

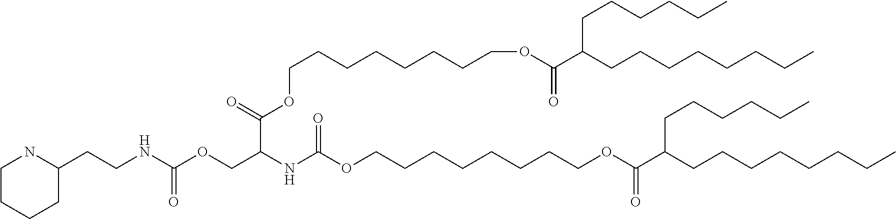

25-hexyl-10,13,24-trioxo-11-((((2-(piperidin-1-yl)ethyl)carbamoyl)oxy)methyl)-9,14,23-trioxa-12-azatritriacontyl 2-hexyldecanoate was synthesized according to representative synthetic scheme 2 and the corresponding methods. MS [ESI]: m/z: [M+H]. calc. 1036.8 obs. 1036.8; ¹H NMR (400 MHz, CDCl₃): δ 0.87 (12H, t, J=6.66 Hz), 1.25 (62H, m), 1.51-1.70 (16H, m), 2.30 (2H, m), 2.38-2.57 (6H, m), 3.29 (2H, s), 4.06 (6H, t, J=6.66 Hz), 4.15 (2H, t, J=6.79 Hz), 4.36 (1H, m), 4.40-4.48 (1H, m), 4.50-4.58 (1H, m), 5.58 (2H, m).

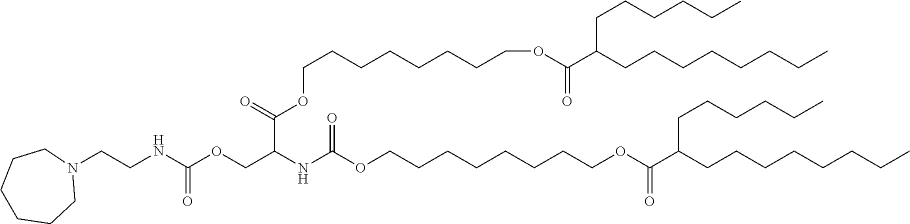 [SMILES: CCCCCCCCC(CCCCCC)C(=O)OCCCCCCCCOC(=O)NC(COC(=O)NCCN1CCCCCC1)C(=O)OCCCCCCCCOC(=O)C(CCCCCC)CCCCCCCC]

11-((((2-(azepan-1-yl)ethyl)carbamoyl)oxy)methyl)-25-hexyl-10,13,24-trioxo-9,14,23-trioxa-12-azatritriacontyl 2-hexyldecanoate was synthesized according to representative synthetic scheme 2 and the corresponding methods. MS [ESI]: m/z: [M+H]. calc. 1050.9 obs. 1051.2; ¹H NMR (400 MHz, CDCl₃): δ 0.87 (12H, t, J=6.62 Hz), 1.25 (60H, m), 1.62 (20H, m), 2.31 (2H, m), 2.59-2.87 (6H, m), 3.29 (2H, m), 4.06 (6H, m), 4.15 (2H, m), 4.29-4.49 (2H, m), 4.49-4.60 (1H, m), 5.61 (2H, s).

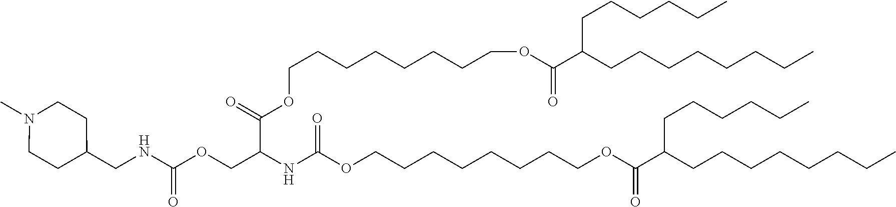

25-hexyl-11-(((((1-methylpiperidin-4-yl)methyl)carbamoyl)oxy)methyl)-10,13,24-trioxo-9,14,23-trioxa-12-azatritriacontyl 2-hexyldecanoate was synthesized according to representative synthetic scheme 2 and the corresponding methods. MS [ESI]: m/z: [M+H]. calc. 1036.8 obs. 1037.4; ¹H NMR (400 MHz, CDCl₃): δ 0.87 (12H, t, J=6.41 Hz), 1.25 (62H, m), 1.61 (15H, m), 2.32 (6H, m), 2.61 (1H, m), 2.86-2.98 (2H, m), 3.07 (2H, m), 4.05 (6H, t, J=6.79 Hz), 4.10-4.18 (2H, m), 4.29-4.46 (2H, m), 4.49-4.59 (1H, m), 4.86 (1H, m), 5.50 (1H, d, J=7.69 Hz).

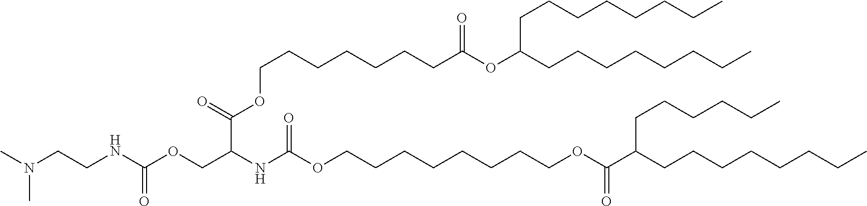

9-(((8-(heptadecan-9-yloxy)-8-oxooctyl)oxy)carbonyl)-2-methyl-6,11-dioxo-7,12-dioxa-2,5,10-triazaicosan-20-yl 2-hexyldecanoate was synthesized according to representative synthetic scheme 2 and the corresponding methods. MS [ESI]: m/z: [M+H]. calc. 1010.8 obs. 1011.5; ¹H NMR (400 MHz, CDCl₃): δ 0.88 (12H, m), 1.25 (74H, m), 2.27 (3H, m), 2.44 (6H, s), 2.59-2.72 (2H, m), 3.37 (2H, m), 3.64 (1H, s), 4.06 (4H, t, J=6.66 Hz), 4.15 (2H, t, J=6.79 Hz), 4.31-4.49 (2H, m), 4.49-4.60 (1H, m), 4.86 (1H, m), 5.61-5.75 (1H, m).

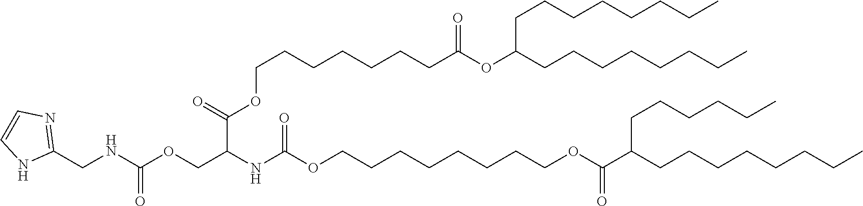

12-(((((1H-imidazol-2-yl)methyl)carbamoyl)oxy)methyl)-24-octyl-10,13,22-trioxo-9,14,23-trioxa-11-azadotriacontyl 2-hexyldecanoate was synthesized according to representative synthetic scheme 2 and the corresponding methods. MS [ESI]: m/z: [M+H]. calc. 1019.8 obs. 1020.5; ¹H NMR (400 MHz, CDCl₃): δ 0.87 (12H, t, J=6.62 Hz), 1.25 (74H, m), 2.28 (2H, m), 2.45 (1H, s), 4.06 (4H, m), 4.10-4.18 (2H, m), 4.24-4.34 (1H, m), 4.38-4.71 (4H, m), 4.86 (1H, m), 5.56-5.70 (1H, m), 6.12 (1H, m), 7.05 (2H, m).

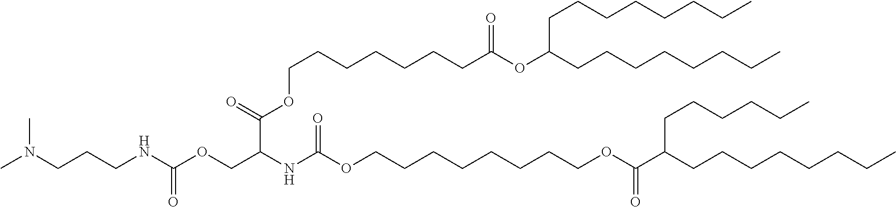

10-(((8-(heptadecan-9-yloxy)-8-oxooctyl)oxy)carbonyl)-2-methyl-7,12-dioxo-8,13-dioxa-2,6,11-triazahenicosan-21-yl 2-hexyldecanoate was synthesized according to representative synthetic scheme 2 and the corresponding methods. MS [ESI]: m/z: [M+H]. calc. 1024.8 obs. 1025.4; ¹H NMR (400 MHz, CDCl₃): δ 0.87 (12H, t, J=6.66 Hz), 1.25 (76H, m), 2.25 (11H, m), 3.20-3.29 (2H, m), 4.06 (4H, t, J=6.62 Hz), 4.14 (2H, t, J=6.70 Hz), 4.29-4.47 (2H, m), 4.49-4.57 (1H, m), 4.85 (1H, m), 5.55 (1H, d, J=7.86 Hz), 5.69 (1H, m).

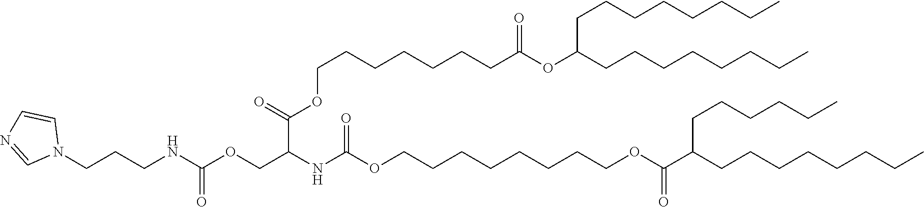

12-((((3-(1H-imidazol-1-yl)propyl)carbamoyl)oxy)methyl)-24-octyl-10,13,22-trioxo-9,14,23-trioxa-11-azadotriacontyl 2-hexyldecanoate was synthesized according to representative synthetic scheme 2 and the corresponding methods. MS [ESI]: m/z: [M+H]. calc. 1047.8 obs. 1047.9; ¹H NMR (400 MHz, CDCl₃): δ 0.87 (12H, t, J=6.58 Hz), 1.25 (74H, m), 2.07-2.19 (2H, m), 2.27 (3H, m), 3.11-3.31 (2H, m), 4.05 (4H, t, J=6.66 Hz), 4.10-4.20 (2H, m), 4.42 (4H, m), 4.56 (1H, m), 4.85 (1H, i, J=6.66 Hz), 5.73-5.91 (1H, m), 6.01-6.25 (1H, m), 7.39 (1H, s).

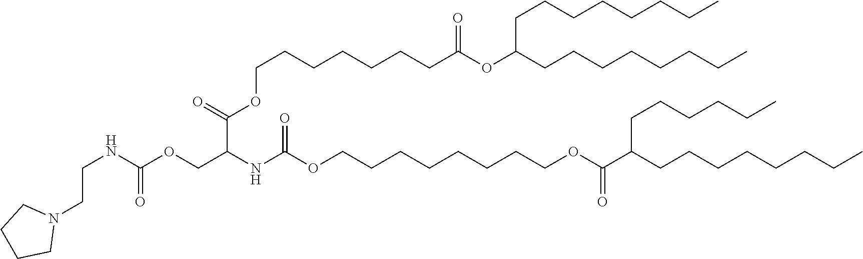

24-octyl-10,13,22-trioxo-12-((((2-(pyrrolidin-1-yl)ethyl)carbamoyl)oxy)methyl)-9,14,23-trioxa-11-azadotriacontyl 2-hexyldecanoate was synthesized according to representative synthetic scheme 2 and the corresponding methods. MS [ESI]: m/z: [M+H]. calc. 1036.8 obs. 1037.3; ¹H NMR (400 MHz, CDCl₃): δ 0.87 (12H, m), 1.25 (78H, m), 2.04-2.18 (4H, m), 2.27 (3H, m), 3.12-3.22 (2H, m), 3.58 (2H, m), 4.05 (4H, t, J=7.01 Hz), 4.09-4.19 (2H, m), 4.31-4.51 (3H, m), 4.51-4.58 (1H, m), 4.84 (1H, m), 5.84-5.94 (1H, m).

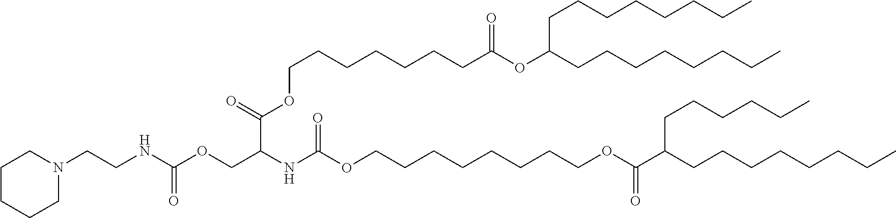

24-octyl-10,13,22-trioxo-12-((((2-(piperidin-1-yl)ethyl)carbamoyl)oxy)methyl)-9,14,23-trioxa-11-azadotriacontyl 2-hexyldecanoate was synthesized according to representative synthetic scheme 2 and the corresponding methods. MS [ESI]: m/z: [M+H]. calc. 1050.8 obs. 1051.2; 1H NMR (400 MHz, CDCl₃): δ 0.87 (12H, t, J=6.53 Hz), 1.25 (80H, m), 1.76-2.14 (4H, m) 2.27 (311, m), 2.83-3.04 (2H, m), 3.48-3.63 (2H, m), 4.06 (4H, t, J=6.49 Hz), 4.14 (2H, t, J=7.01 Hz), 4.28-4.49 (2H, m), 4.49-4.59 (1H, m), 4.85 (1H, i, J=7.00 Hz), 5.76 (1H, m).

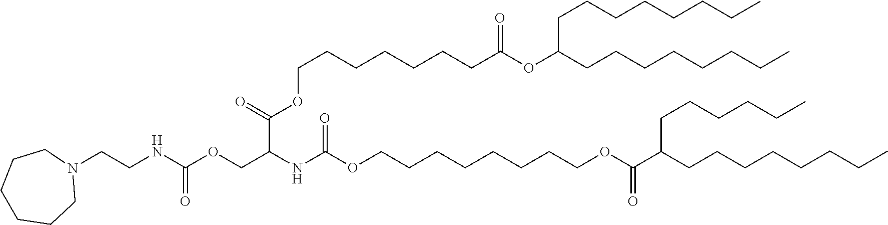 [SMILES: CCCCCCCCC(CCCCCCCC)OC(=O)CCCCCCCOC(=O)C(COC(=O)NCCN1CCCCCC1)NC(=O)OCCCCCCCCOC(=O)C(CCCCCC)CCCCCCCC]

12-((((2-(azepan-1-yl)ethyl)carbamoyl)oxy)methyl)-24-octyl-10,13,22-trioxo-9,14,23-trioxa-11-azadotriacontyl 2-hexyldecanoate was synthesized according to representative synthetic scheme 2 and the corresponding methods. MS [ESI]: m/z: [M+H]. calc. 1064.9 obs. 1065.5; ¹H NMR (400 MHz, CDCl₃): δ 0.87 (12H, t, J=6.58 Hz), 1.25 (82H, m), 2.27 (3H, m), 2.62-2.85 (6H, m), 3.38 (2H, m), 4.06 (4H, dd, J=8.53, 6.70 Hz), 4.15 (2H, dd, J=7.72, 6.84 Hz), 4.31-4.49 (2H, m), 4.49-4.59 (1H, m), 4.86 (1H, m), 5.54-5.70 (1H, m).

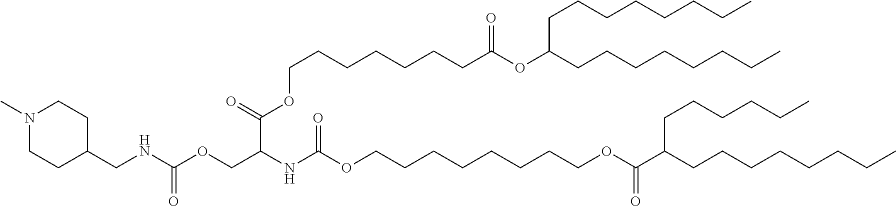

12-(((((1-methylpiperidin-4-yl)methyl)carbamoyl)oxy)methyl)-24-octyl-10,13,22-trioxo-9,14,23-trioxa-11-azadotriacontyl 2-hexyldecanoate was synthesized according to representative synthetic scheme 2 and the corresponding methods. MS [ESI]: m/z: [M+H]. calc. 1050.9 obs. 1051.5; ¹H NMR (400 MHz, CDCl₃): δ 0.87 (12H, t, J=6.58 Hz), 1.25 (79H, m), 1.93-2.07 (2H, m), 2.31 (6H, m), 2.86-2.96 (2H, m), 3.06 (2H, m), 4.06 (4H, m), 4.14 (2H, m), 4.29-4.46 (2H, m), 4.53 (1H, m), 4.86 (2H, m), 5.50 (1H, m).

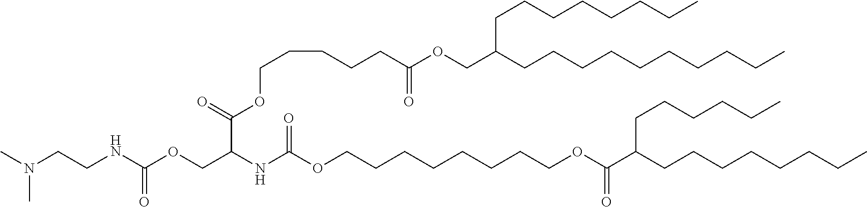

2-methyl-9-(((6-((2-octyldodecyl)oxy)-6-oxohexyl)oxy)carbonyl)-6,11-dioxo-7,12-dioxa-2,5,10-triazaicosan-20-yl 2-hexyldecanoate hexyldecanoate was synthesized according to representative synthetic scheme 2 and the corresponding methods. MS [ESI]: m/z: [M+H]. calc. 1024.8 obs. 1025.4; ¹H NMR (400 MHz, CDCl₃): δ 0.87 (12H, m), 1.26 (62H, m), 1.51-1.74 (13H, m), 2.31 (3H, m), 2.69 (6H, s), 2.96 (2H, m), 3.51 (2H, m), 3.96 (2H, d, J=5.81 Hz), 4.06 (4H, t, J=6.66 Hz), 4.15 (2H, t, J=6.49 Hz), 4.32-4.50 (2H, m), 4.50-4.59 (1H, m), 5.90 (1H, m), 6.20-6.50 (1H, m).

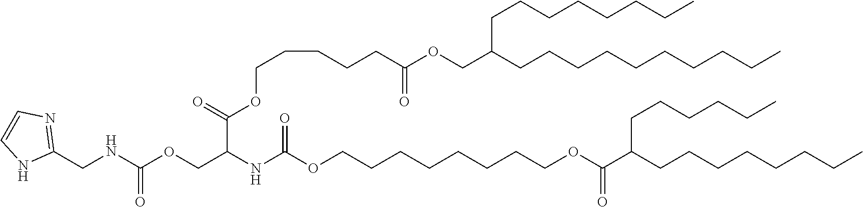

12-(((((1H-imidazol-2-yl)methyl)carbamoyl)oxy)methyl)-23-octyl-10,13,20-trioxo-9,14,21-trioxa-11-azatritriacontyl 2-hexyldecanoate was synthesized according to representative synthetic scheme 2 and the corresponding methods. MS [ESI]: m/z: [M+H]. calc. 1033.8 obs. 1034.1; ¹H NMR (400 MHz, CDCl₃): δ 0.88 (12H, m), 1.26 (58H, m), 1.54-1.73 (11H, m), 1.73-1.89 (611, m), 2.25-2.39 (3H, m), 3.97 (2H, d, J=5.98 Hz), 4.06 (4H, t, J=6.87 Hz), 4.10-4.22 (2H, m), 4.22-4.33 (111, m), 4.39-4.50 (1H, m), 4.64-4.72 (1H, m), 4.78-4.93 (2H, m), 5.73-5.82 (1H, m), 6.97-7.09 (1H, m), 7.20 (2H, m).

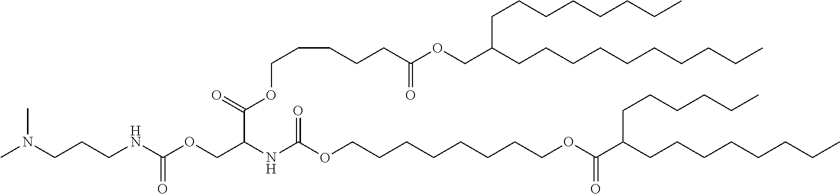

2-methyl-10-(((6-((2-octyldodecyl)oxy)-6-oxohexyl)oxy)carbonyl)-7,12-dioxo-8,13-dioxa-2,6,11-triazahenicosan-21-yl 2-hexyldecanoate was synthesized according to representative synthetic scheme 2 and the corresponding methods. MS [ESI]: m/z: [M+H]. calc. 1038.9 obs. 1039.7; ¹H NMR (400 MHz, CDCl₃): δ 0.87 (12H, t, J=6.58 Hz), 1.26 (62H, m), 1.51-1.74 (13H, m), 1.96-2.11 (2H, m), 2.32 (3H, m), 2.74 (6H, s), 2.94-3.06 (2H, m), 3.28-3.41 (2H, m), 3.96 (2H, d, J=5.81 Hz), 4.06 (4H, t, J=6.66 Hz), 4.16 (2H, m), 4.31-4.50 (2H, m), 4.50-4.59 (1H, m), 5.65 (1H, d, J=8.52 Hz), 5.91 (1H, m).

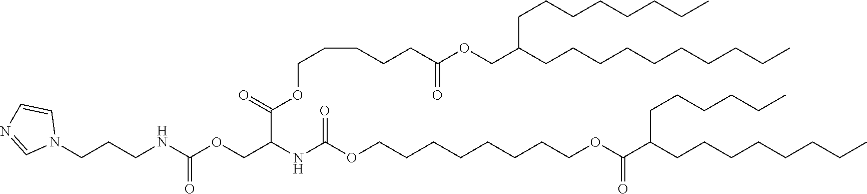

12-((((3-(1H-imidazol-1-yl)propyl)carbamoyl)oxy)methyl)-23-octyl-10,13,20-trioxo-9,14,21-trioxa-11-azatritriacontyl 2-hexyldecanoate was synthesized according to representative synthetic scheme 2 and the corresponding methods. MS [ESI]: m/z: [M+H]. calc. 1061.8 obs. 1062.3; ¹H NMR (400 MHz, CDCl₃): δ 0.87 (12H, m), 1.26 (64H, m), 1.61 (11H, m), 2.03-2.13 (3H, m), 2.31 (3H, m), 3.11-3.26 (2H, m), 3.95 (2H, d, J=5.64 Hz), 4.05 (4H, t, J=6.80 Hz), 4.11-4.21 (2H, m), 4.23-4.32 (2H, m), 4.35-4.45 (2H, m), 4.51-4.59 (1H, m), 5.71-5.80 (1H, m), 5.81-5.91 (1H, m), 7.16 (1H, m), 7.29 (1H, t, J=1.88 Hz), 8.96 (1H, m).

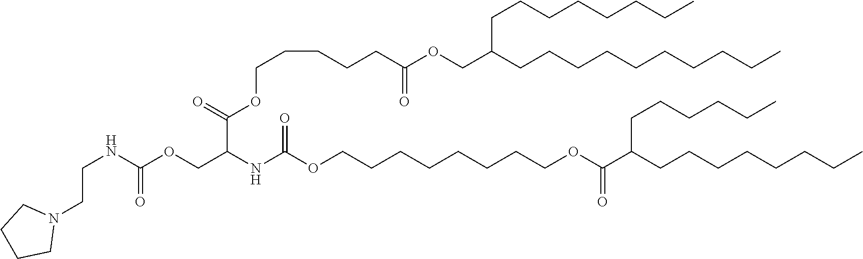

23-octyl-10,13,20-trioxo-12-((((2-(pyrrolidin-1-yl)ethyl)carbamoyl)oxy)methyl)-9,14,21-trioxa-11-azatritriacontyl 2-hexyldecanoate was synthesized according to representative synthetic scheme 2 and the corresponding methods. MS [ESI]: m/z: [M+H]. calc. 1050.9 obs. 1051.0; ¹H NMR (400 MHz, CDCl₃): δ 0.87 (12H, t, J=6.41 Hz), 1.26 (62H, m), 1.50-1.84 (17H, m), 2.09 (2H, s), 2.32 (5H, m), 2.82 (2H, m), 3.61 (2H, m), 3.96 (2H, d, J=5.98 Hz), 4.05 (4H, dd, J=7.50, 6.86 Hz), 4.16 (2H, m), 4.29-4.51 (2H, m), 4.51-4.59 (1H, m), 5.94 (1H, m), 6.69 (1H, m).

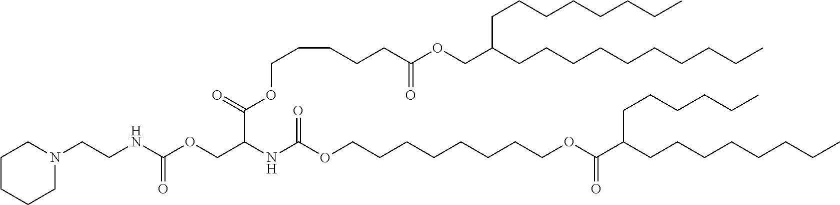

23-octyl-10,13,20-trioxo-12-((((2-(piperidin-1-yl)ethyl)carbamoyl)oxy)methyl)-9,14,21-trioxa-11-azatritriacontyl 2-hexyldecanoate was synthesized according to representative synthetic scheme 2 and the corresponding methods. MS [ESI]: m/z: [M+H]. calc. 1064.9 obs. 1065.1; ¹H NMR (400 MHz, CDCl₃): δ 0.88 (12H, m), 1.26 (55H, m), 1.37-1.47 (4H, m), 1.51-1.74 (15H, m), 1.77-1.99 (2H, m), 2.31 (3H, m), 2.51-2.83 (2H, m), 3.06 (2H, m), 3.62 (4H, m), 3.96 (2H, d, J=5.81 Hz), 4.06 (4H, t, J=6.66 Hz), 4.16 (2H, t, J=6.96 Hz), 4.29-4.51 (2H, m), 4.51-4.59 (1H, m), 5.84 (1H, d, J=8.20 Hz), 6.80-7.02 (1H, m).

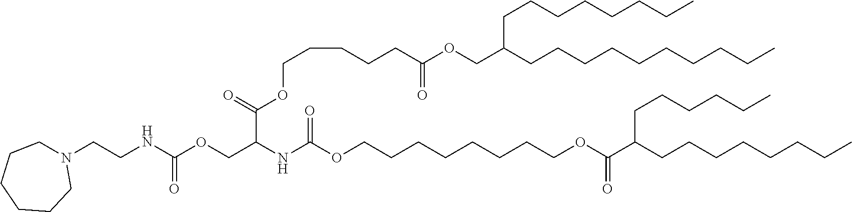

12-((((2-(azepan-1-yl)ethyl)carbamoyl)oxy)methyl)-23-octyl-10,13,20-trioxo-9,14,21-trioxa-11-azatritriacontyl 2-hexyldecanoate was synthesized according to representative synthetic scheme 2 and the corresponding methods. MS [ESI]: m/z: [M+H]. calc. 1078.9 obs. 1080.5; ¹H NMR (400 MHz, CDCl₃): δ 0.87 (12H, m), 1.26 (62H, m), 1.60 (17H, m), 1.78-1.92 (4H, m), 2.10-2.24 (2H, m), 2.31 (3H, m), 2.90-3.05 (2H, m), 3.15 (2H, m), 3.55-3.69 (2H, m), 3.96 (2H, d, J=5.72 Hz), 4.06 (4H, t, J=6.90 Hz), 4.16 (2H, t, J=6.77 Hz), 4.29-4.50 (2H, m), 4.51-4.58 (1H, m), 5.84 (1H, d, J=8.76 Hz), 6.90 (1H, m).

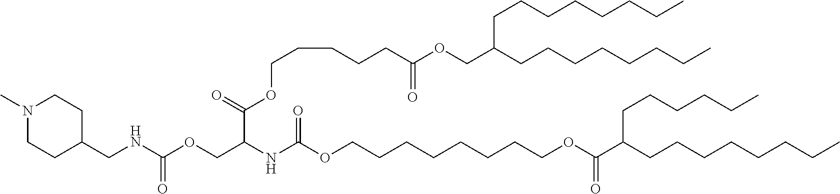

12-(((((1-methylpiperidin-4-yl)methyl)carbamoyl)oxy)methyl)-23-octyl-10,13,20-trioxo-9,14,21-trioxa-11-azatritriacontyl 2-hexyldecanoate was synthesized according to representative synthetic scheme 2 and the corresponding methods. MS [ESI]: m/z: [M+H]. calc. 1064.9 obs. 1065.5; ¹H NMR (400 MHz, CDCl₃): δ 0.87 (12H, m), 1.26 (62H, m), 1.51-1.75 (16H, m), 1.78-1.91 (2H, m), 2.31 (3H, t, J=7.75 Hz), 2.58 (3H, s), 3.04-3.14 (2H, m), 3.17-3.30 (2H, m), 3.38 (2H, m), 3.95 (2H, d, J=5.72 Hz), 4.06 (4H, t, J=6.62 Hz), 4.11-4.22 (2H, m), 4.38 (2H, m), 4.48-4.58 (1H, m), 5.17-5.25 (1H, m), 5.54 (1H, d, J=7.52 Hz).

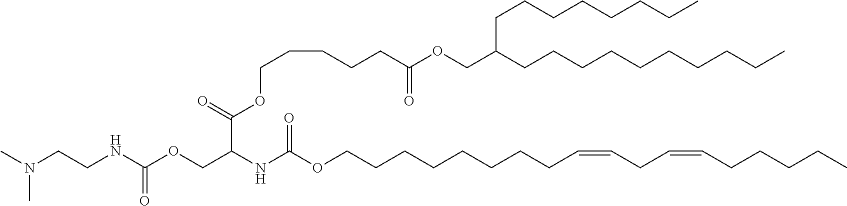 [SMILES: CCCCC/C=C\C/C=C\CCCCCCCCOC(=O)NC(COC(=O)NCCN(C)C)C(=O)OCCCCCC(=O)OCC(CCCCCCCC)CCCCCCCCCC]

2-octyldodecyl 6-((O-((2-(dimethylamino)ethyl)carbamoyl)-N-((((9Z,12Z)-octadeca-9,12-dien-1-yl)oxy)carbonyl)seryl)oxy)hexanoate was synthesized according to representative synthetic scheme 2 and the corresponding methods. MS [ESI]: m/z: [M+H]. calc. 906.7 obs. 907.4; ¹H NMR (400 MHz, CDCl₃): δ 0.88 (9H, m), 1.26 (47H, m), 1.56-1.75 (10H, m), 2.05 (4H, q, J=7.34 Hz), 2.30 (8H, m), 2.48 (2H, m), 2.77 (2H, dd, J=7.39, 6.53 Hz), 3.28 (211, m), 3.96 (2H, d, J=5.89 Hz), 4.06 (2H, dd, J=7.99, 6.75 Hz), 4.16 (2H, t, J=6.70 Hz), 4.30-4.48 (2H, m), 4.54 (1H, m), 536 (4H, m), 5.44-5.54 (1H, m), 5.60 (1H, d, J=9.221 Hz).

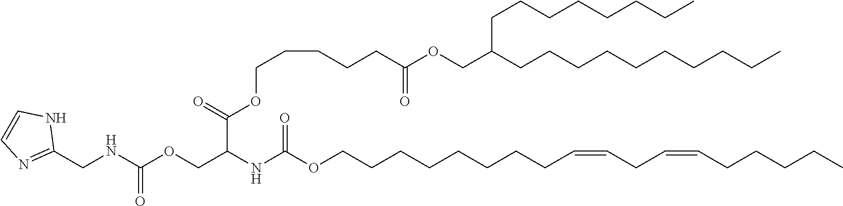 [SMILES: CCCCC/C=C\C/C=C\CCCCCCCCOC(=O)NC(COC(=O)NCc1ncc[nH]1)C(=O)OCCCCCC(=O)OCC(CCCCCCCC)CCCCCCCCCC]

2-octyldodecyl 6-((O-(((1H-imidazol-2-yl)methyl)carbamoyl)-N-((((9Z,12Z)-octadeca-9,12-dien-1-yl)oxy)carbonyl)seryl)oxy)hexanoate was synthesized according to representative synthetic scheme 2 and the corresponding methods. MS [ESI]: m/z: [M+H]. calc. 915.7 obs. 916.3; ¹H NMR (400 MHz, CDCl₃): δ 0.90 (9H, m), 1.28 (47H, m), 1.63 (10H, m), 2.06 (4H, m), 2.33 (2H, t, J=7.34 Hz), 2.79 (2H, t, J=6.28 Hz), 3.98 (2H, d, J=5.81 Hz), 4.01-4.27 (4H, m), 4.46 (4H, m), 4.65 (1H, m), 5.37 (4H, m), 5.59 (1H, d, J=8.11 Hz), 5.93 (1H, m), 7.02 (2H, m).

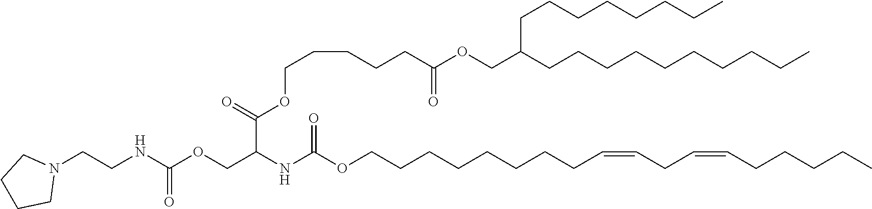 [SMILES: CCCCC/C=C\C/C=C\CCCCCCCCOC(=O)NC(COC(=O)NCCN1CCCC1)C(=O)OCCCCCC(=O)OCC(CCCCCCCC)CCCCCCCCCC]

2-octyldodecyl 6-((N-((((9Z,12Z)-octadeca-9,12-dien-1-yl)oxy)carbonyl)-O-((2-(pyrrolidin-1-yl)ethyl)carbamoyl)seryl)oxy)hexanoate was synthesized according to representative synthetic scheme 2 and the corresponding methods. MS [ESI]: m/z: [M+H]. calc. 932.8 obs. 933.5; ¹H NMR (400 MHz, CDCl₃): δ 0.88 (9H, m), 1.26 (48H, m), 1.53-1.74 (8H, m), 1.78-1.88 (5H, m), 2.04 (4H, q, J=7.17 Hz), 2.31 (2H, dd, J=7.86, 7.09 Hz), 2.54-2.72 (6H, m), 2.77 (2H, dd, J=7.46, 6.58 Hz), 3.32 (2H, m), 3.96 (2H, d, J=5.81 Hz), 4.06 (2H, dd, J=8.03, 6.68 Hz), 4.16 (2H, t, J=6.80 Hz), 4.31-4.48 (2H, m), 4.49-4.60 (1H, m), 5.35 (4H, m), 5.45-5.68 (2H, m).

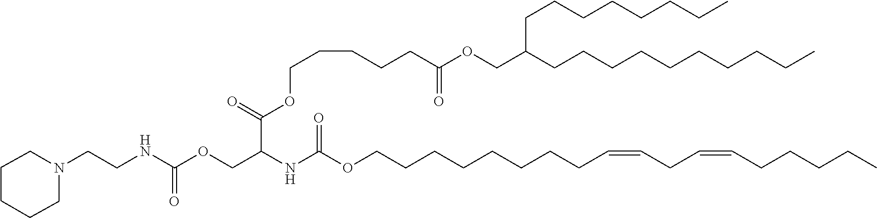 [SMILES: CCCCC/C=C\C/C=C\CCCCCCCCOC(=O)NC(COC(=O)NCCN1CCCCC1)C(=O)OCCCCCC(=O)OCC(CCCCCCCC)CCCCCCCCCC]

2-octyldodecyl 6-((N-((((9Z,12Z)-octadeca-9,12-dien-1-yl)oxy)carbonyl)-O-((2-(piperidin-1-yl)ethyl)carbamoyl)seryl)oxy)hexanoate was synthesized according to representative synthetic scheme 2 and the corresponding methods. MS [ESI]: m/z: [M+H]. calc. 946.8 obs. 947.6; ¹H NMR (400 MHz, CDCl₃): δ 0.90 (9H, m), 1.28 (51H, m), 1.65 (12H, m), 2.06 (4H, m), 2.33 (3H, m), 2.39-2.58 (5H, m), 2.79 (2H, dd, J=7.82, 6.90 Hz), 3.31 (2H, s), 3.98 (2H, d, J=5.81 Hz), 4.18 (4H, m), 4.32-4.51 (2H, m), 4.51-4.60 (1H, m), 5.37 (4H, m), 5.56-5.68 (1H, m).

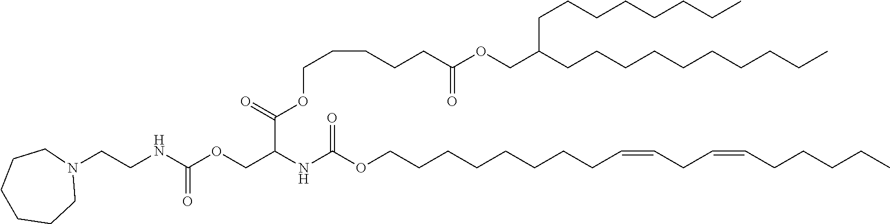 [SMILES: CCCCC/C=C\C/C=C\CCCCCCCCOC(=O)NC(COC(=O)NCCN1CCCCCC1)C(=O)OCCCCCC(=O)OCC(CCCCCCCC)CCCCCCCCCC]

2-octyldodecyl 6-((O-((2-(azepan-1-yl)ethyl)carbamoyl)-N-((((9Z,12Z)-octadeca-9,12-dien-1-yl)oxy)carbonyl)seryl)oxy)hexanoate was synthesized according to representative synthetic scheme 2 and the corresponding methods. MS [ESI]: m/z: [M+H]. calc. 960.8 obs. 961.5; ¹H NMR (400 MHz, CDCl₃): δ 0.88 (9H, m), 1.26 (48H, m), 1.63 (17H, m), 2.04 (5H, m), 2.31 (2H, t, J=7.43 Hz), 2.77 (7H, m), 3.25 (2H, m), 3.96 (2H, d, J=5.81 Hz), 4.16 (4H, m), 4.29-4.49 (2H, m), 4.49-4.60 (1H, m), 5.35 (4H, m), 5.59 (1H, m).

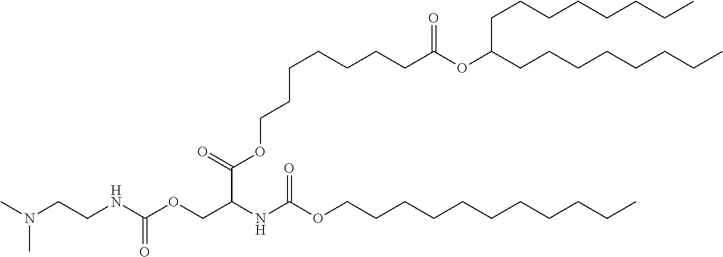 [SMILES: CCCCCCCCCCCOC(=O)NC(COC(=O)NCCN(C)C)C(=O)OCCCCCCCC(=O)OC(CCCCCCCC)CCCCCCCC]

heptadecan-9-yl 8-((O-((2-(dimethylamino)ethyl)carbamoyl)-N-((undecyloxy)carbonyl)seryl)oxy)octanoate was synthesized according to representative synthetic scheme 2 and the corresponding methods. MS [ESI]: m/z: [M+H]. calc. 798.6 obs. 799.5; ¹H NMR (400 MHz, CDCl₃): δ 0.88 (9H, m), 1.26 (46H, m), 1.44-1.55 (4H, m), 1.55-1.69 (6H, m), 2.30 (8H, m), 2.49 (2H, m), 3.28 (2H, br s), 4.06 (2H, t, J=6.36 Hz), 4.15 (2H, t, J=6.75 Hz), 4.29-4.49 (2H, m), 4.49-4.60 (1H, m), 4.86 (1H, i, J=6.66 Hz), 5.33-5.51 (1H, m), 5.59 (1H, d, J=8.46 Hz).

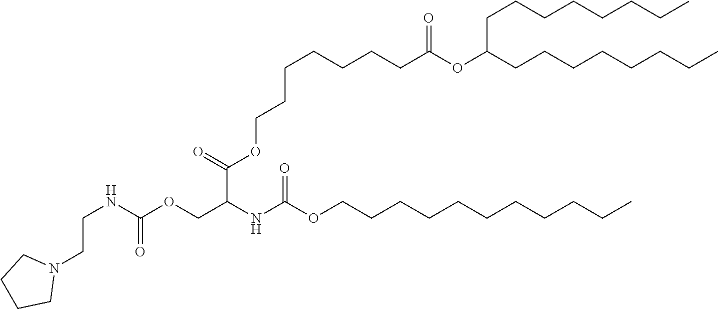 [SMILES: CCCCCCCCCCCOC(=O)NC(COC(=O)NCCN1CCCC1)C(=O)OCCCCCCCC(=O)OC(CCCCCCCC)CCCCCCCC]

heptadecan-9-yl 8-((O-((2-(pyrrolidin-1-yl)ethyl)carbamoyl)-N-((undecyloxy)carbonyl)seryl)oxy)octanoate was synthesized according to representative synthetic scheme 2 and the corresponding methods. MS [ESI]: m/z: [M+H]. calc. 824.7 obs. 825.3; ¹H NMR (400 MHz, CDCl₃): δ 0.88 (9H, m), 1.26 (44H, m), 1.45-1.55 (4H, m), 1.56-1.70 (8H, m), 1.78 (4H, m), 2.27 (2H, dd, J=7.94, 7.09 Hz), 2.47-2.55 (4H, m), 2.55-2.63 (2H, m), 3.22-3.32 (2H, m), 4.03-4.10 (2H, m), 4.15 (2H, t, J=6.90 Hz), 4.30-4.47 (2H, m), 4.50-4.60 (1H, m), 4.86 (1H, i, J=6.66 Hz), 5.27-5.39 (1H, m), 5.55 (1H, m).

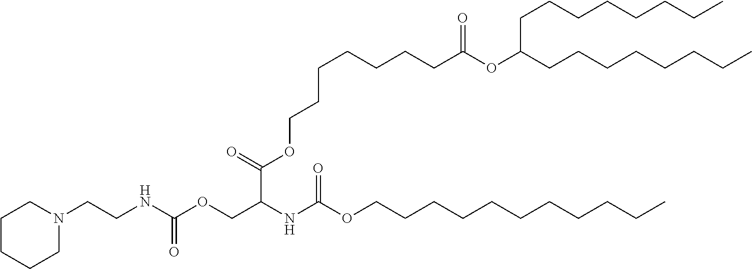 [SMILES: CCCCCCCCCCCOC(=O)NC(COC(=O)NCCN1CCCCC1)C(=O)OCCCCCCCC(=O)OC(CCCCCCCC)CCCCCCCC]

heptadecan-9-yl 8-((O-((2-(piperidin-1-yl)ethyl)carbamoyl)-N-((undecyloxy)carbonyl)seryl)oxy)octanoate was synthesized according to representative synthetic scheme 2 and the corresponding methods. MS [ESI]: m/z: [M+H]. calc. 838.7 obs. 839.0; ¹H NMR (400 MHz, CDCl₃): δ 0.88 (9H, t, J=6.83 Hz), 1.26 (46H, m), 1.56 (16H, m), 2.27 (2H, dd, J=7.86, 7.17 Hz), 2.32-2.61 (6H, m), 3.22-3.37 (2H, m), 4.06 (2H, dd, J=8.03, 7.06 Hz), 4.15 (2H, t, J=6.97 Hz), 4.30-4.48 (2H, m), 4.50-4.60 (1H, m), 4.85 (1H, i, J=6.66 Hz), 5.30 (1H, s), 5.53-5.64 (1H, m).

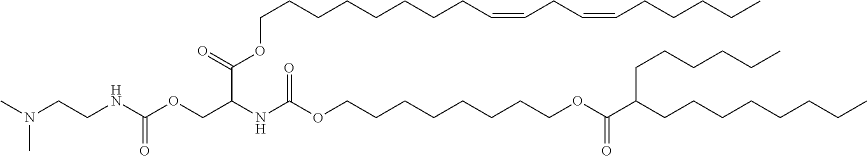 [SMILES: CCCCC/C=C\C/C=C\CCCCCCCCOC(=O)C(COC(=O)NCCN(C)C)NC(=O)OCCCCCCCCOC(=O)C(CCCCCC)CCCCCCCC]

2-methyl-9-((((9Z,12Z)-octadeca-9,12-dien-1-yl)oxy)carbonyl)-6,11-dioxo-7,12-dioxa-2,5,10-triazaicosan-20-yl 2-hexyldecanoate was synthesized according to representative synthetic scheme 2 and the corresponding methods. MS [ESI]: m/z: [M+H]. calc. 878.7 obs. 879.0; ¹H NMR (400 MHz, CDCl₃): δ 0.88 (9H, m), 1.25 (46H, m), 1.66 (8H, m), 2.05 (4H, q, J=7.34 Hz), 2.25 (7H, m), 2.42 (2H, m), 2.77 (2H, td, J=6.41, 1.11 Hz), 3.21-3.30 (2H, m), 4.06 (4H, t, J=6.66 Hz), 4.15 (2H, t, J=6.92 Hz), 4.31-4.47 (2H, m), 4.50-4.60 (1H, m), 5.27-5.43 (4H, m), 5.51-5.62 (1H, m).

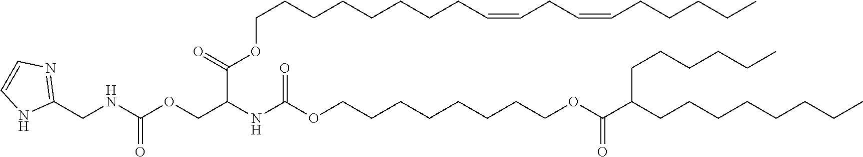 [SMILES: CCCCC/C=C\C/C=C\CCCCCCCCOC(=O)C(COC(=O)NCc1ncc[nH]1)NC(=O)OCCCCCCCCOC(=O)C(CCCCCC)CCCCCCCC]

1-(1H-imidazol-2-yl)-6-((((9Z,12Z)-octadeca-9,12-dien-1-yl)oxy)carbonyl)-3,8-dioxo-4,9-dioxa-2,7-diazaheptadecan-17-yl 2-hexyldecanoate was synthesized according to representative synthetic scheme 2 and the corresponding methods. MS [ESI]: m/z: [M+H]. calc. 887.7 obs. 888.1; ¹H NMR (400 MHz, CDCl₃): δ 0.88 (9H, m), 1.25 (46H, m), 1.50-1.72 (8H, m), 2.05 (4H, q, J=7.60 Hz), 2.30 (1H, m), 2.77 (2H, t, J=6.49 Hz), 4.06 (4H, m), 4.11-4.21 (2H, m), 4.28-4.53 (4H, m), 4.60-4.70 (1H, m), 5.35 (4H, m), 5.44-5.55 (2H, m), 6.97 (2H, m).

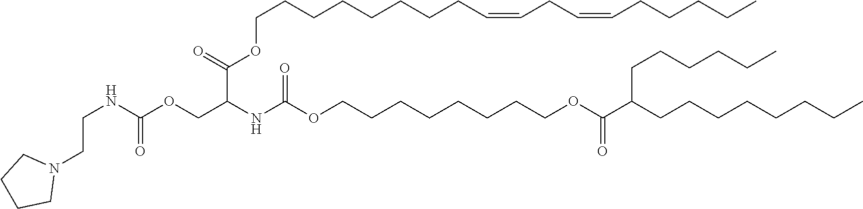 [SMILES: CCCCC/C=C\C/C=C\CCCCCCCCOC(=O)C(COC(=O)NCCN1CCCC1)NC(=O)OCCCCCCCCOC(=O)C(CCCCCC)CCCCCCCC]

7-((((9Z,12Z)-octadeca-9,12-dien-1-yl)oxy)carbonyl)-4,9-dioxo-1-(pyrrolidin-1-yl)-5,10-dioxa-3,8-diazaoctadecan-18-yl 2-hexyldecanoate was synthesized according to representative synthetic scheme 2 and the corresponding methods. MS [ESI]: m/z: [M+H]. calc. 904.7 obs. 904.9; ¹H NMR (400 MHz, CDCl₃): δ 0.88 (9H, m), 1.25 (46H, m), 1.66 (8H, m), 1.77 (4H, m), 2.05 (4H, q, J=7.30 Hz), 2.30 (1H, m), 2.50 (6H, m), 2.77 (2H, dd, J=7.35, 6.45 Hz), 3.23-3.30 (2H, m), 4.06 (4H, dd, J=7.88, 6.76 Hz), 4.15 (2H, t, J=6.84 Hz), 4.30-4.48 (2H, m), 4.49-4.58 (1H, m), 5.35 (5H, m), 5.50-5.60 (1H, m).

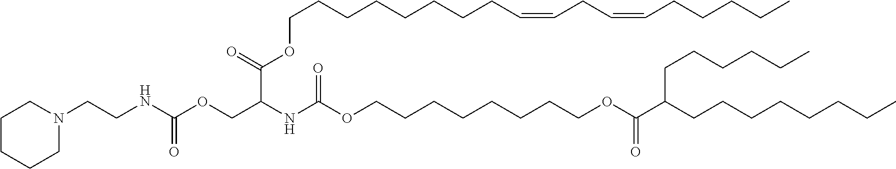 [SMILES: CCCCC/C=C\C/C=C\CCCCCCCCOC(=O)C(COC(=O)NCCN1CCCCC1)NC(=O)OCCCCCCCCOC(=O)C(CCCCCC)CCCCCCCC]

7-((((9Z,12Z)-octadeca-9,12-dien-1-yl)oxy)carbonyl)-4,9-dioxo-1-(piperidin-1-yl)-5,10-dioxa-3,8-diazaoctadecan-18-yl 2-hexyldecanoate was synthesized according to representative synthetic scheme 2 and the corresponding methods. MS [ESI]: m/z: [M+H]. calc. 918.7 obs. 919.1; ¹H NMR (400 MHz, CDCl₃): δ 0.88 (9H, m), 1.25 (46H, m), 1.59 (14H, m), 2.05 (4H, q, J=6.83 Hz), 2.24-2.45 (7H, m), 2.77 (2H, t, J=6.62 Hz), 3.23 (2H, m), 4.06 (4H, m), 4.16 (2H, t, J=6.98 Hz), 4.28-4.49 (3H, m), 4.49-4.62 (1H, m), 5.36 (4H, m), 5.54 (1H, m).

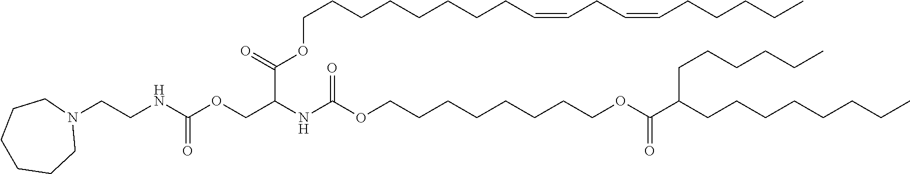 [SMILES: CCCCC/C=C\C/C=C\CCCCCCCCOC(=O)C(COC(=O)NCCN1CCCCCC1)NC(=O)OCCCCCCCCOC(=O)C(CCCCCC)CCCCCCCC]

1-(azepan-1-yl)-7-((((9Z,12Z)-octadeca-9,12-dien-1-yl)oxy)carbonyl)-4,9-dioxo-5,10-dioxa-3,8-diazaoctadecan-18-yl 2-hexyldecanoate was synthesized according to representative synthetic scheme 2 and the corresponding methods. MS [ESI]: m/z: [M+H]. calc. 932.8 obs. 933.4; ¹H NMR (400 MHz, CDCl₃): δ 0.87 (9H, m), 1.25 (46H, m), 1.59 (16H, m), 2.05 (4H, q, J=7.17 Hz), 2.26-2.35 (1H, m), 2.52-2.66 (6H, m), 2.77 (2H, t, J=6.11 Hz), 3.20 (2H, m), 4.06 (4H, t, J=6.62 Hz), 4.16 (2H, t, J=6.83 Hz), 4.28-4.49 (2H, m), 4.49-4.60 (1H, m), 5.35 (5H, m), 5.55 (1H, d, J=7.77 Hz).

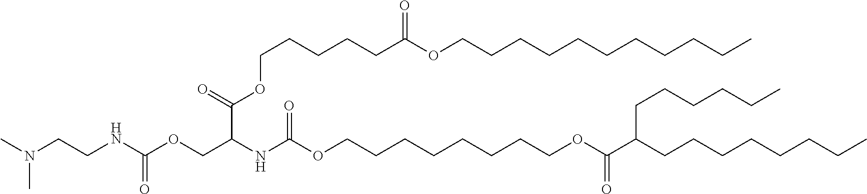 [SMILES: CCCCCCCCCCCOC(=O)CCCCCOC(=O)C(COC(=O)NCCN(C)C)NC(=O)OCCCCCCCCOC(=O)C(CCCCCC)CCCCCCCC]

2-methyl-6,11-dioxo-9-(((6-oxo-6-(undecyloxy)hexyl)oxy)carbonyl)-7,12-dioxa-2,5,10-triazaicosan-20-yl 2-hexyldecanoate was synthesized according to representative synthetic scheme 2 and the corresponding methods. MS [ESI]: m/z: [M+H]. calc. 898.7 obs. 899.2; ¹H NMR (400 MHz, CDCl₃): δ 0.88 (9H, m), 1.26 (44H, m), 1.58 (16H, m), 2.22 (6H, s), 2.30 (4H, t, J=7.86 Hz), 2.35-2.43 (1H, m), 3.24 (2H, m), 4.06 (6H, td, J=6.66, 2.56 Hz), 4.17 (2H, td, J=6.58, 1.54 Hz), 4.29-4.47 (2H, m), 4.48-4.60 (1H, m), 5.25-5.36 (1H, m), 5.51-5.61 (1H, m).

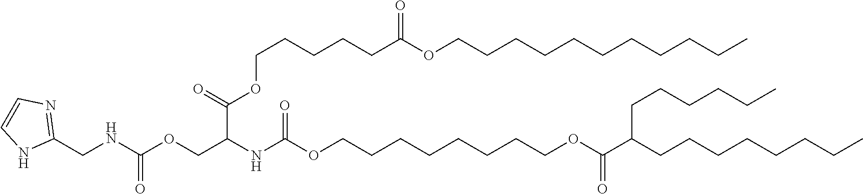 [SMILES: CCCCCCCCCCCOC(=O)CCCCCOC(=O)C(COC(=O)NCc1ncc[nH]1)NC(=O)OCCCCCCCCOC(=O)C(CCCCCC)CCCCCCCC]

12-(((((1H-imidazol-2-yl)methyl)carbamoyl)oxy)methyl)-10,13,20-trioxo-9,14,21-trioxa-11-azadotriacontyl 2-hexyldecanoate was synthesized according to representative synthetic scheme 2 and the corresponding methods. MS [ESI]: m/z: [M+H]. calc. 907.7 obs. 908.1; ¹H NMR (400 MHz, CDCl₃): δ 0.87 (9H, t, J=6.53 Hz), 1.26 (48H, m), 1.61 (12H, m), 2.30 (3H, t, J=7.09 Hz), 4.06 (8H, m), 4.30-4.51 (411, m), 4.62 (1H, m), 5.55 (1H, d, J=7.69 Hz), 5.69-5.82 (1H, m), 6.97 (2H, s).

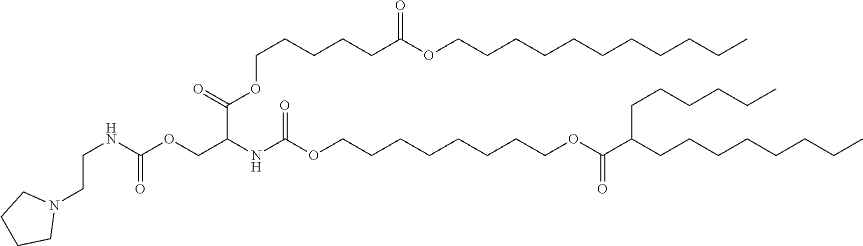 [SMILES: CCCCCCCCCCCOC(=O)CCCCCOC(=O)C(COC(=O)NCCN1CCCC1)NC(=O)OCCCCCCCCOC(=O)C(CCCCCC)CCCCCCCC]

10,13,20-trioxo-12-((((2-(pyrrolidin-1-yl)ethyl)carbamoyl)oxy)methyl)-9,14,21-trioxa-11-azadotriacontyl 2-hexyldecanoate was synthesized according to representative synthetic scheme 2 and the corresponding methods. MS [ESI]: m/z: [M+H]. calc. 924.7 obs. 924.9; ¹H NMR (400 MHz, CDCl₃): δ 0.87 (9H, m), 1.26 (48H, m), 1.67 (12H, m), 1.77 (4H, m), 2.30 (3H, t, J=7.86 Hz), 2.51 (6H, m), 3.26 (2H, m), 4.06 (6H, td, J=6.53, 2.45 Hz), 4.16 (2H, td, J=6.45, 1.88 Hz), 4.29-4.48 (2H, m), 4.55 (1H, m), 5.36 (1H, m), 5.57 (1H, d, J=7.69 Hz).

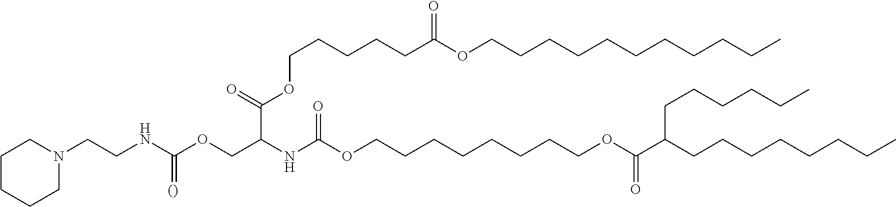

10,13,20-trioxo-12-((((2-(piperidin-1-yl)ethyl)carbamoyl)oxy)methyl)-9,14,21-trioxa-11-azadotriacontyl 2-hexyldecanoate was synthesized according to representative synthetic scheme 2 and the corresponding methods. MS [ESI]: m/z: [M+H]. calc. 938.7 obs. 939.0; ¹H NMR (400 MHz, CDCl₃): δ 0.87 (9H, m), 1.26 (48H, m), 1.50-1.74 (18H, m), 2.30 (9H, m), 3.25 (2H, d, J=6.83 Hz), 4.05 (6H, td, J=6.66, 2.82 Hz), 4.17 (2H, td, J=6.49, 1.88 Hz), 4.30-4.48 (2H, m), 4.49-4.59 (1H, m), 5.36 (1H, m), 5.55-5.62 (1H, m).

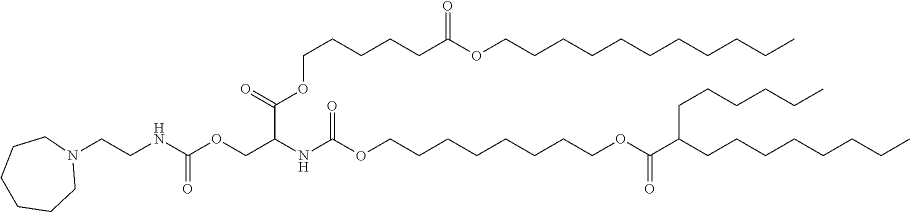 [SMILES: CCCCCCCCCCCOC(=O)CCCCCOC(=O)C(COC(=O)NCCN1CCCCCC1)NC(=O)OCCCCCCCCOC(=O)C(CCCCCC)CCCCCCCC]

12-((((2-(azepan-1-yl)ethyl)carbamoyl)oxy)methyl)-10,13,20-trioxo-9,14,21-trioxa-11-azadotriacontyl 2-hexyldecanoate was synthesized according to representative synthetic scheme 2 and the corresponding methods. MS [ESI]: m/z: [M+H]. calc. 952.8 obs. 952.9; ¹H NMR (400 MHz, CDCl₃): δ 0.87 (9H, m), 1.26 (48H, m), 1.50-1.74 (20H, m), 2.30 (9H, m), 3.19-3.30 (2H, m), 4.05 (6H, td, J=6.66, 2.82 Hz), 4.17 (2H, td, J=6.49, 1.54 Hz), 4.30-4.48 (2H, m), 4.49-4.59 (1H, m), 5.31-5.41 (1H, m), 5.58 (1H, d, J=8.20 Hz).

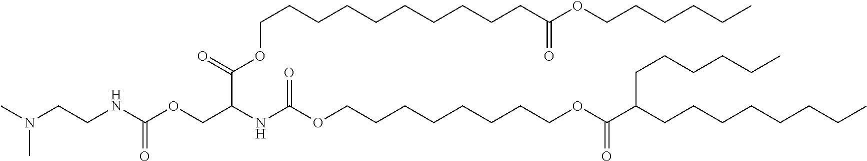 [SMILES: CCCCCCCCC(CCCCCC)C(=O)OCCCCCCCCOC(=O)NC(COC(=O)NCCN(C)C)C(=O)OCCCCCCCCCCC(=O)OCCCCCC]

hexyl 11-((O-((2-(dimethylamino)ethyl)carbamoyl)-N-(((8-((2-hexyldecanoyl)oxy)octyl)oxy)carbonyl)seryl)oxy)undecanoate was synthesized according to representative synthetic scheme 2 and the corresponding methods. MS [ESI]: m/z: [M+H]. calc. 898.7 obs. 899.2; ¹H NMR (400 MHz, CDCl₃): δ 0.88 (9H, m), 1.25 (48H, m), 1.61 (12H, m), 2.24 (6H, s), 2.29 (3H, m), 2.42 (2H, t, J=6.32 Hz), 3.25 (2H, m), 4.06 (6H, t, J=6.70 Hz), 4.15 (2H, t, J=6.77 Hz), 4.34 (1H, dd, J=11.57, 3.26 Hz), 4.43 (1H, dd, J=11.57, 3.69 Hz), 4.54 (1H, m), 5.35 (1H, m), 5.56 (1H, d, J=7.64 Hz).

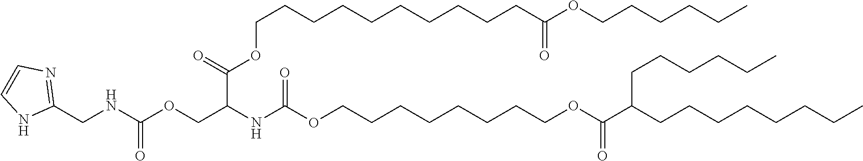 [SMILES: CCCCCCCCC(CCCCCC)C(=O)OCCCCCCCCOC(=O)NC(COC(=O)NCc1ncc[nH]1)C(=O)OCCCCCCCCCCC(=O)OCCCCCC]

hexyl 11-((O-(((1H-imidazol-2-yl)methyl)carbamoyl)-N-(((8-((2-hexyldecanoyl)oxy)octyl)oxy)carbonyl)seryl)oxy)undecanoate was synthesized according to representative synthetic scheme 2 and the corresponding methods. MS [ESI]: m/z: [M+H]. calc. 907.7 obs. 908.4; ¹H NMR (400 MHz, CDCl₃): δ 0.88 (9H, m), 1.25 (48H, m), 1.61 (12H, m), 2.29 (3H, m), 4.06 (6H, t, J=6.75 Hz), 4.15 (2H, t, J=6.75 Hz), 4.40 (4H, m), 4.63 (1H, m), 5.54 (2H, m), 6.97 (2H, s).

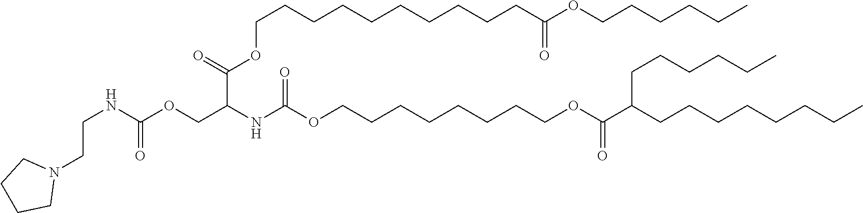 [SMILES: CCCCCCCCC(CCCCCC)C(=O)OCCCCCCCCOC(=O)NC(COC(=O)NCCN1CCCC1)C(=O)OCCCCCCCCCCC(=O)OCCCCCC]

hexyl 11-((N-(((8-((2-hexyldecanoyl)oxy)octyl)oxy)carbonyl)-O-((2-(pyrrolidin-1-yl)ethyl)carbamoyl)seryl)oxy)undecanoate was synthesized according to representative synthetic scheme 2 and the corresponding methods. MS [ESI]: m/z: [M+H]. calc. 924.7 obs. 925.0; ¹H NMR (400 MHz, CDCl₃): δ 0.88 (9H, m), 1.25 (48H, m), 1.61 (16H, m), 2.28 (3H, m), 2.51 (6H, m), 3.27 (2H, m), 4.06 (6H, t, J=6.62 Hz), 4.15 (2H, t, J=6.63 Hz), 4.34 (1H, dd, J=11.04, 3.07 Hz), 4.43 (1H, dd, J=11.04, 4.22 Hz), 4.54 (1H, m), 5.35 (1H, m), 5.56 (1H, d, J=8.06 Hz).

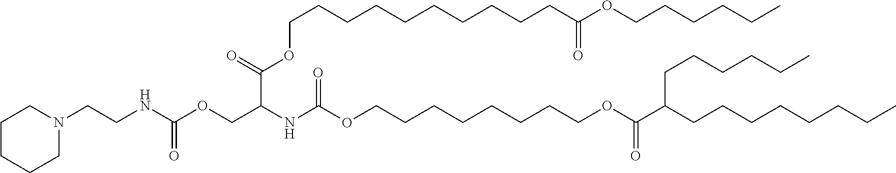 [SMILES: CCCCCCCCC(CCCCCC)C(=O)OCCCCCCCCOC(=O)NC(COC(=O)NCCN1CCCCC1)C(=O)OCCCCCCCCCCC(=O)OCCCCCC]

hexyl 11-((N-(((8-((2-hexyldecanoyl)oxy)octyl)oxy)carbonyl)-O-((2-(piperidin-1-yl)ethyl)carbamoyl)seryl)oxy)undecanoate was synthesized according to representative synthetic scheme 2 and the corresponding methods. MS [ESI]: m/z: [M+H]. calc. 938.7 obs. 940.2; ¹H NMR (400 MHz, CDCl₃): δ 0.88 (9H, m), 1.25 (50H, m), 1.62 (16H, m), 2.28 (9H, m), 3.24 (2H, m), 4.06 (6H, t, J=6.95 Hz), 4.15 (2H, t, J=6.65 Hz), 4.34 (1H, dd, J=11.30, 3.83 Hz), 4.44 (1H, dd, J=11.30, 4.63 Hz), 4.55 (1H, m), 5.28 (1H, m), 5.55 (1H, d, J=8.18 Hz).

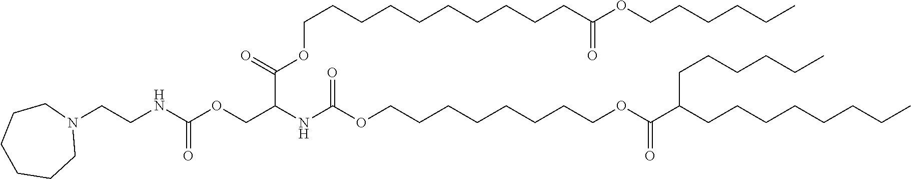 [SMILES: CCCCCCCCC(CCCCCC)C(=O)OCCCCCCCCOC(=O)NC(COC(=O)NCCN1CCCCCC1)C(=O)OCCCCCCCCCCC(=O)OCCCCCC]

hexyl 11-((O-((2-(azepan-1-yl)ethyl)carbamoyl)-N-(((8-((2-hexyldecanoyl)oxy)octyl)oxy)carbonyl)seryl)oxy)undecanoate was synthesized according to representative synthetic scheme 2 and the corresponding methods. MS [ESI]: m/z: [M+H]. calc. 952.8 obs. 953.3; ¹H NMR (400 MHz, CDCl₃): δ 0.88 (9H, m), 1.25 (48H, m), 1.61 (20H, m), 2.28 (3H, m), 2.60 (6H, m), 3.20 (2H, m), 4.06 (611, t, J=6.66 Hz), 4.15 (2H, t, J=6.68 Hz), 4.34 (1H, dd, J=11.37, 2.50 Hz), 4.44 (1H, dd, J=11.37, 3.88 Hz), 4.54 (1H, m), 5.30 (111, m), 5.55 (1H, d, J=8.16 Hz).

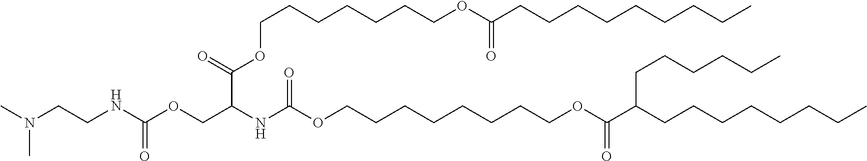 [SMILES: CCCCCCCCCC(=O)OCCCCCCCOC(=O)C(COC(=O)NCCN(C)C)NC(=O)OCCCCCCCCOC(=O)C(CCCCCC)CCCCCCCC]

9-(((7-(decanoyloxy)heptyl)oxy)carbonyl)-2-methyl-6,11-dioxo-7,12-dioxa-2,5,10-triazaicosan-20-yl 2-hexyldecanoate was synthesized according to representative synthetic scheme 2 and the corresponding methods. MS [ESI]: m/z: [M+H]. calc. 898.7 obs. 899.0; ¹H NMR (400 MHz, CDCl₃): δ 0.87 (9H, m), 1.25 (46H, m), 1.67 (14H, m), 2.22 (6H, s), 2.29 (3H, m), 2.40 (2H, t, J=6.25 Hz), 3.23 (2H, m), 4.05 (6H, m), 4.16 (2H, t, J=6.89 Hz), 4.34 (1H, dd, J=11.27, 3.14 Hz), 4.44 (1H, dd, J=11.27, 4.17 Hz), 4.54 (1H, m), 5.29 (1H, m), 5.55 (1H, d, J=8.15 Hz).

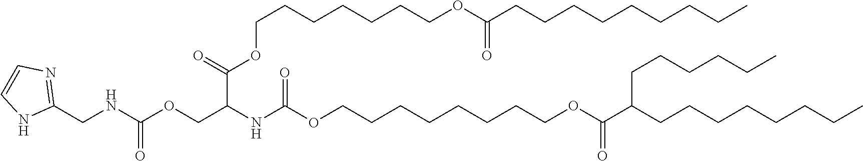 [SMILES: CCCCCCCCCC(=O)OCCCCCCCOC(=O)C(COC(=O)NCc1ncc[nH]1)NC(=O)OCCCCCCCCOC(=O)C(CCCCCC)CCCCCCCC]

12-(((((1H-imidazol-2-yl)methyl)carbamoyl)oxy)methyl)-10,13,23-trioxo-9,14,22-trioxa-11-azadotriacontyl 2-hexyldecanoate was synthesized according to representative synthetic scheme 2 and the corresponding methods. MS [ESI]: m/z: [M+H]. calc. 907.7 obs. 907.8; ¹H NMR (400 MHz, CDCl₃): δ 0.87 (9H, m), 1.25 (48H, m), 1.61 (12H, m), 2.29 (3H, m), 4.05 (6H, m), 4.15 (2H, m), 4.40 (4H, m), 4.64 (1H, m), 5.53 (1H, d, J=8.23 Hz), 5.60 (1H, m), 6.97 (2H, s).

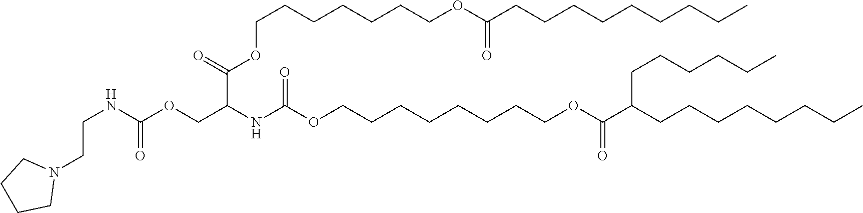 [SMILES: CCCCCCCCCC(=O)OCCCCCCCOC(=O)C(COC(=O)NCCN1CCCC1)NC(=O)OCCCCCCCCOC(=O)C(CCCCCC)CCCCCCCC]

10,13,23-trioxo-12-((((2-(pyrrolidin-1-yl)ethyl)carbamoyl)oxy)methyl)-9,14,22-trioxa-11-azadotriacontyl 2-hexyldecanoate was synthesized according to representative synthetic scheme 2 and the corresponding methods. MS [ESI]: m/z: [M+H]. calc. 924.7 obs. 925.4; ¹H NMR (400 MHz, CDCl₃): δ 0.87 (9H, m), 1.25 (46H, m), 1.61 (18H, m), 2.29 (3H, m), 2.55 (4H, m), 2.61 (2H, m), 3.28 (2H, m), 4.05 (6H, m), 4.15 (2H, t, J=6.68 Hz), 4.34 (1H, dd, J=11.50, 3.34 Hz), 4.44 (1H, dd, J=11.50, 4.19 Hz), 4.54 (1H, m), 5.45 (1H, m), 5.59 (1H, d, J=8.30 Hz).

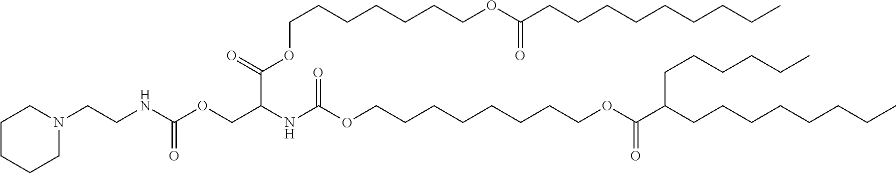 [SMILES: CCCCCCCCCC(=O)OCCCCCCCOC(=O)C(COC(=O)NCCN1CCCCC1)NC(=O)OCCCCCCCCOC(=O)C(CCCCCC)CCCCCCCC]

10,13,23-trioxo-12-((((2-(piperidin-1-yl)ethyl)carbamoyl)oxy)methyl)-9,14,22-trioxa-11-azadotriacontyl 2-hexyldecanoate was synthesized according to representative synthetic scheme 2 and the corresponding methods. MS [ESI]: m/z: [M+H]. calc. 938.7 obs. 938.9; ¹H NMR (400 MHz, CDCl₃): δ 0.87 (9H, m), 1.25 (48H, m), 1.63 (18H, m), 2.29 (9H, m), 3.24 (2H, m), 4.05 (6H, m), 4.16 (2H, t, J=6.74 Hz), 4.34 (1H, dd, J=11.64, 3.33 Hz), 4.45 (1H, dd, J=11.64, 4.01 Hz), 4.55 (1H, m), 5.30 (1H, m), 5.56 (1H, d, J=8.61 Hz).

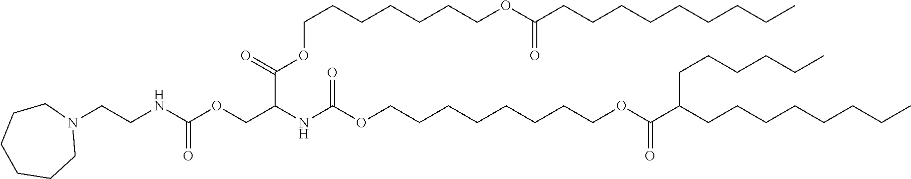 [SMILES: CCCCCCCCCC(=O)OCCCCCCCOC(=O)C(COC(=O)NCCN1CCCCCC1)NC(=O)OCCCCCCCCOC(=O)C(CCCCCC)CCCCCCCC]

12-((((2-(azepan-1-yl)ethyl)carbamoyl)oxy)methyl)-10,13,23-trioxo-9,14,22-trioxa-11-azadotriacontyl 2-hexyldecanoate was synthesized according to representative synthetic scheme 2 and the corresponding methods. MS [ESI]: m/z: [M+H]. calc. 952.8 obs. 953.1; ¹H NMR (400 MHz, CDCl₃): δ 0.87 (9H, m), 1.25 (46H, m), 1.61 (22H, m), 2.29 (3H, m), 2.60 (6H, m), 3.20 (2H, m), 4.05 (6H, m), 4.16 (2H, t, J=6.72 Hz), 4.34 (1H, dd, J=10.97, 3.15 Hz), 4.45 (1H, dd, J=10.97, 3.49 Hz), 4.54 (1H, m), 5.34 (1H, m), 5.57 (1H, d, J=8.27 Hz).

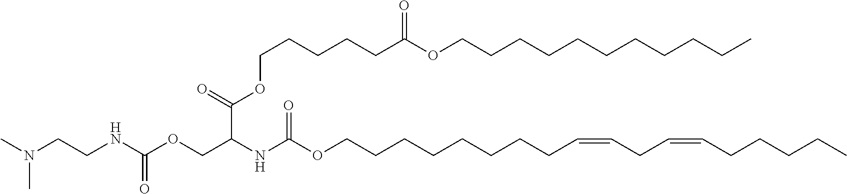

undecyl 6-((O-((2-(dimethylamino)ethyl)carbamoyl)-N-((((9Z,12Z)-octadeca-9,12-dien-1-yl)oxy)carbonyl)seryl)oxy)hexanoate was synthesized according to representative synthetic scheme 2 and the corresponding methods. MS [ESI]: m/z: [M+H]. calc. 780.6 obs. 781.2; ¹H NMR (400 MHz, CDCl₃): δ 0.88 (6H, m), 1.26 (34H, m), 1.63 (8H, m), 2.05 (4H, q, J=7.53 Hz), 2.23 (6H, s), 2.30 (2H, t, J=7.53 Hz), 2.41 (2H, t, J=5.95 Hz), 2.77 (2H, t, J=6.89 Hz), 3.25 (2H, m), 4.05 (4H, m), 4.16 (2H, m), 4.34 (1H, m), 4.44 (1H, m), 4.54 (1H, m), 5.35 (5H, m), 5.58 (1H, m).

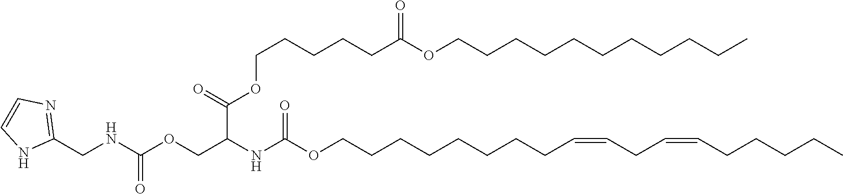 [SMILES: CCCCC/C=C\C/C=C\CCCCCCCCOC(=O)NC(COC(=O)NCc1ncc[nH]1)C(=O)OCCCCCC(=O)OCCCCCCCCCCC]

undecyl 6-((O-(((1H-imidazol-2-yl)methyl)carbamoyl)-N-((((9Z,12Z)-octadeca-9,12-dien-1-yl)oxy)carbonyl)seryl)oxy)hexanoate was synthesized according to representative synthetic scheme 2 and the corresponding methods. MS [ESI]: m/z: [M+H]. calc. 789.6 obs. 790.2; ¹H NMR (400 MHz, CDCl₃): δ 0.88 (6H, m), 1.26 (34H, m), 1.63 (8H, m), 2.05 (4H, q, J=7.22 Hz), 2.30 (2H, t, J=7.26 Hz), 2.77 (2H, t, J=6.55 Hz), 4.05 (4H, m), 4.17 (2H, m), 4.40 (4H, m), 4.63 (1H, m), 5.35 (4H, m), 5.54 (1H, m), 5.72 (1H, m), 6.96 (2H, s).

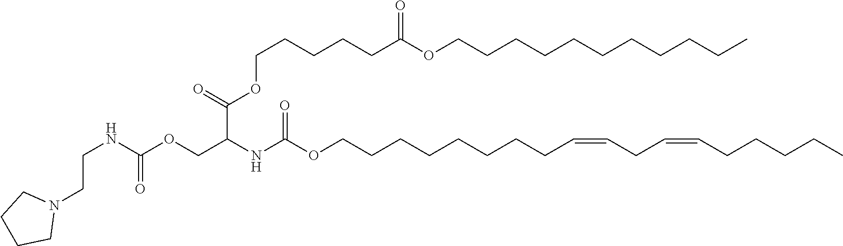 [SMILES: CCCCC/C=C\C/C=C\CCCCCCCCOC(=O)NC(COC(=O)NCCN1CCCC1)C(=O)OCCCCCC(=O)OCCCCCCCCCCC]

undecyl 6-((N-((((9Z,12Z)-octadeca-9,12-dien-1-yl)oxy)carbonyl)-O-((2-(pyrrolidin-1-yl)ethyl)carbamoyl)seryl)oxy)hexanoate was synthesized according to representative synthetic scheme 2 and the corresponding methods. MS [ESI]: m/z: [M+H]. calc. 806.6 obs. 807.4; ¹H NMR (400 MHz, CDCl₃): δ 0.88 (6H, m), 1.26 (34H, m), 1.69 (12H, m), 2.05 (4H, q, J=7.37 Hz), 2.30 (2H, t, J=7.52 Hz), 2.52 (4H, m), 2.59 (2H, t, J=6.06 Hz), 2.77 (2H, t, J=6.89 Hz), 3.27 (2H, m), 4.05 (4H, m), 4.16 (2H, m), 4.34 (1H, m), 4.44 (1H, m), 4.54 (1H, m), 5.35 (5H, m), 5.58 (1H, d, J=8.62 Hz).

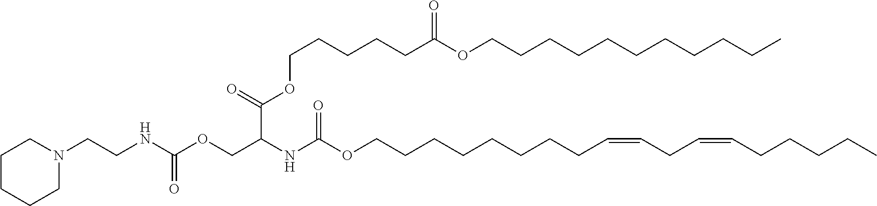 [SMILES: CCCCC/C=C\C/C=C\CCCCCCCCOC(=O)NC(COC(=O)NCCN1CCCCC1)C(=O)OCCCCCC(=O)OCCCCCCCCCCC]

undecyl 6-((N-((((9Z,12Z)-octadeca-9,12-dien-1-yl)oxy)carbonyl)-O-((2-(piperidin-1-yl)ethyl)carbamoyl)seryl)oxy)hexanoate was synthesized according to representative synthetic scheme 2 and the corresponding methods. MS [ESI]: m/z: [M+H]. calc. 820.6 obs. 821.3; ¹H NMR (400 MHz, CDCl₃): δ 0.86 (6H, m), 1.24 (34H, m), 1.60 (14H, m), 2.03 (4H, q, J=6.92 Hz), 2.33 (8H, m), 2.75 (2H, t, J=6.40 Hz), 3.22 (2H, m), 4.03 (4H, m), 4.15 (2H, t, J=6.65 Hz), 4.32 (1H, dd, J=11.33, 3.05 Hz), 4.42 (1H, dd, J=11.33, 3.11 Hz), 4.53 (1H, m), 5.34 (5H, m), 5.55 (1H, d, J=7.87 Hz).

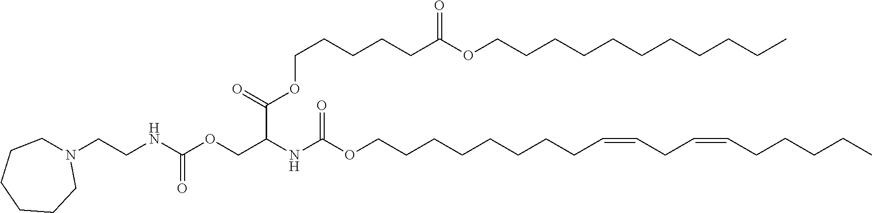 [SMILES: CCCCC/C=C\C/C=C\CCCCCCCCOC(=O)NC(COC(=O)NCCN1CCCCCC1)C(=O)OCCCCCC(=O)OCCCCCCCCCCC]

undecyl 6-((O-((2-(azepan-1-yl)ethyl)carbamoyl)-N-((((9Z,12Z)-octadeca-9,12-dien-1-yl)oxy)carbonyl)seryl)oxy)hexanoate was synthesized according to representative synthetic scheme 2 and the corresponding methods. MS [ESI]: m/z: [M+H]. calc. 834.7 obs. 835.6; ¹H NMR (400 MHz, CDCl₃): δ 0.88 (6H, m), 1.26 (34H, m), 1.59 (16H, m), 2.05 (4H, q, J=7.26 Hz), 2.30 (2H, t, J=7.52 Hz), 2.62 (6H, m), 2.77 (2H, t, J=6.54 Hz), 3.20 (2H, m), 4.05 (4H, m), 4.17 (2H, t, J=6.79 Hz), 4.34 (1H, m), 4.44 (1H, m), 4.55 (1H, m), 5.36 (5H, m), 5.57 (1H, m).

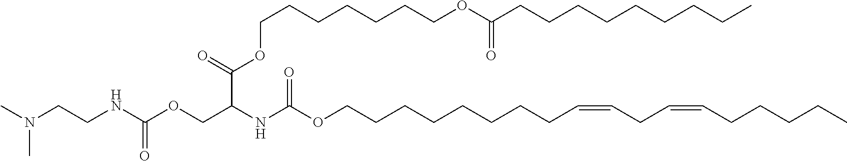 [SMILES: CCCCC/C=C\C/C=C\CCCCCCCCOC(=O)NC(COC(=O)NCCN(C)C)C(=O)OCCCCCCCOC(=O)CCCCCCCCC]

2-methyl-9-(((((9Z,12Z)-octadeca-9,12-dien-1-yl)oxy)carbonyl)amino)-6,10-dioxo-7,11-dioxa-2,5-diazaoctadecan-18-yl decanoate was synthesized according to representative synthetic scheme 2 and the corresponding methods. MS [ESI]: m/z: [M+H]. calc. 780.6 obs. 781.2; ¹H NMR (400 MHz, CDCl₃): δ 0.88 (6H, m), 1.20-1.41 (34H, m), 1.62 (8H, m), 2.05 (4H, q, J=7.13 Hz), 2.22 (6H, s), 2.29 (2H, t, J=7.94 Hz), 2.39 (2H, t, J=6.32 Hz), 2.77 (2H, t, J=6.98 Hz), 3.19-3.28 (2H, m), 4.05 (4H, m), 4.15 (2H, t, J=6.70 Hz), 4.30-4.48 (2H, m), 4.49-4.59 (1H, m), 5.35 (5H, m), 5.54 (1H, d, J=7.86 Hz).

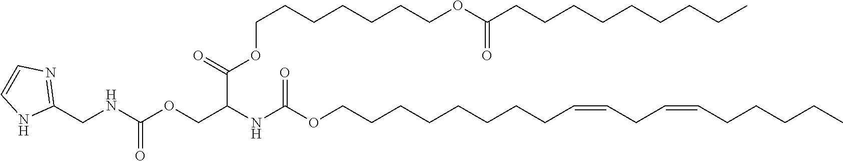 [SMILES: CCCCC/C=C\C/C=C\CCCCCCCCOC(=O)NC(COC(=O)NCc1ncc[nH]1)C(=O)OCCCCCCCOC(=O)CCCCCCCCC]

7-((O-(((1H-imidazol-2-yl)methyl)carbamoyl)-N-((((9Z,12Z)-octadeca-9,12-dien-1-yl)oxy)carbonyl)seryl)oxy)heptyl decanoate was synthesized according to representative synthetic scheme 2 and the corresponding methods. MS [ESI]: m/z: [M+H]. calc. 789.6 obs. 789.9; ¹H NMR (400 MHz, CDCl₃): δ 0.88 (6H, m), 1.30 (34H, m), 1.61 (8H, m), 2.05 (4H, q, J=7.22 Hz), 2.29 (2H, t, J=7.94 Hz), 2.77 (2H, t, J=6.60 Hz), 4.05 (4H, t, J=6.91 Hz), 4.16 (2H, t, J=6.78 Hz), 4.28-4.53 (4H, m), 4.64 (1H, s), 5.35 (4H, m), 5.48-5.59 (1H, m), 6.97 (2H, s).

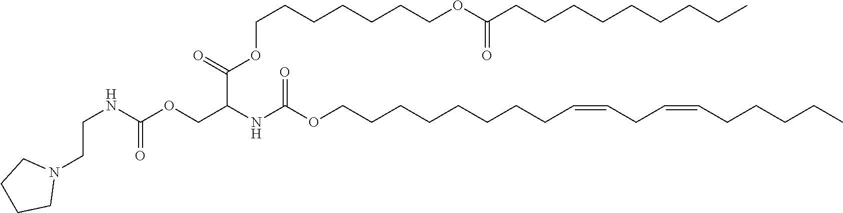 [SMILES: CCCCC/C=C\C/C=C\CCCCCCCCOC(=O)NC(COC(=O)NCCN1CCCC1)C(=O)OCCCCCCCOC(=O)CCCCCCCCC]

7-((N-((((9Z,12Z)-octadeca-9,12-dien-1-yl)oxy)carbonyl)-O-((2-(pyrrolidin-1-yl)ethyl)carbamoyl)seryl)oxy)heptyl decanoate was synthesized according to representative synthetic scheme 2 and the corresponding methods. MS [ESI]: m/z: [M+H]. calc. 806.6 obs. 806.9; ¹H NMR (400 MHz, CDCl₃): δ 0.88 (6H, m), 1.31 (34H, m), 1.66 (12H, m), 2.05 (4H, q, J=7.29 Hz), 2.28 (2H, t, J=7.60 Hz), 2.51 (4H, m), 2.58 (2H, t, J=6.31 Hz), 2.77 (2H, t, J=6.44 Hz), 3.27 (2H, m), 4.06 (4H, m), 4.16 (2H, t, J=6.85 Hz), 4.34 (1H, m), 4.44 (1H, m), 4.55 (1H, m), 5.35 (5H, m), 5.56 (1H, d, J=8.36 Hz).

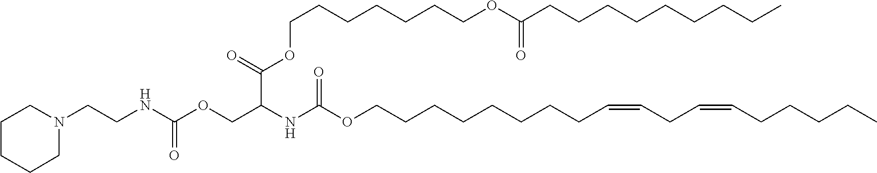 [SMILES: CCCCC/C=C\C/C=C\CCCCCCCCOC(=O)NC(COC(=O)NCCN1CCCCC1)C(=O)OCCCCCCCOC(=O)CCCCCCCCC]

7-((N-((((9Z,12Z)-octadeca-9,12-dien-1-yl)oxy)carbonyl)-O-((2-(piperidin-1-yl)ethyl)carbamoyl)seryl)oxy)heptyl decanoate was synthesized according to representative synthetic scheme 2 and the corresponding methods. MS [ESI]: m/z: [M+H]. calc. 820.6 obs. 821.2; ¹H NMR (400 MHz, CDCl₃): δ 0.88 (6H, m), 1.30 (36H, m), 1.61 (12H, m), 2.05 (4H, q, J=6.83 Hz), 2.29 (2H, t, J=7.62 Hz), 2.38 (6H, m), 2.77 (2H, t, J=6.44 Hz), 3.24 (2H, m), 4.06 (4H, m), 4.16 (2H, t, J=6.86 Hz), 4.34 (1H, dd, J=11.46, 3.96 Hz), 4.44 (1H, dd, J=11.46, 4.03 Hz), 4.55 (1H, m), 5.36 (5H, m), 5.55 (1H, d, J=8.23 Hz).

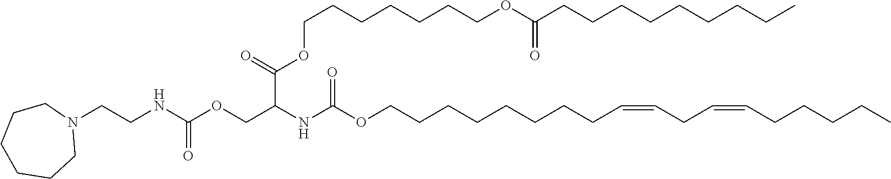 [SMILES: CCCCC/C=C\C/C=C\CCCCCCCCOC(=O)NC(COC(=O)NCCN1CCCCCC1)C(=O)OCCCCCCCOC(=O)CCCCCCCCC]

7-((O-((2-(azepan-1-yl)ethyl)carbamoyl)-N-((((9Z,12Z)-octadeca-9,12-dien-1-yl)oxy)carbonyl)seryl)oxy)heptyl decanoate was synthesized according to representative synthetic scheme 2 and the corresponding methods. MS [ESI]: m/z: [M+H]. calc. 834.7 obs. 834.9; ¹H NMR (400 MHz, CDCl₃): δ 0.88 (6H, m), 1.30 (34H, m), 1.61 (16H, m), 2.04 (4H, q, J=6.83 Hz), 2.28 (2H, t, J=7.74 Hz), 2.64 (6H, m), 2.77 (2H, t, J=6.58 Hz), 3.21 (2H, m), 4.06 (4H, m), 4.15 (2H, t, J=6.92 Hz), 4.34 (1H, m), 4.44 (1H, m), 4.55 (1H, m), 5.35 (5H, m), 5.57 (1H, d, J=8.07 Hz).

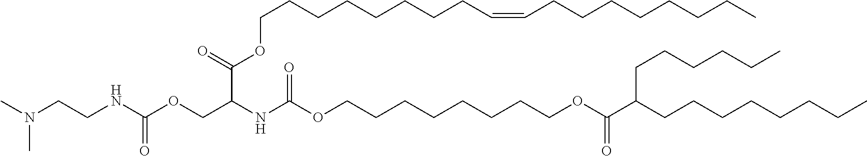 [SMILES: CCCCCCCC/C=C\CCCCCCCCOC(=O)C(COC(=O)NCCN(C)C)NC(=O)OCCCCCCCCOC(=O)C(CCCCCC)CCCCCCCC]

(Z)-2-methyl-9-((octadec-9-en-1-yloxy)carbonyl)-6,11-dioxo-7,12-dioxa-2,5,10-triazaicosan-20-yl 2-hexyldecanoate was synthesized according to representative synthetic scheme 2 and the corresponding methods. MS [ESI]: m/z: [M+H]. calc. 880.7 obs. 881.3; ¹H NMR (400 MHz, CDCl₃): δ 0.87 (9H, m), 1.25 (52H, m), 1.61 (8H, m), 2.01 (4H, m), 2.26 (7H, m), 2.45 (2H, t, J=6.09 Hz), 3.26 (2H, m), 4.06 (4H, t, J=6.63 Hz), 4.15 (2H, t, J=6.85 Hz), 4.35 (1H, dd, J=11.39, 3.00 Hz), 4.43 (1H, dd, J=11.39, 3.93 Hz), 4.54 (1H, m), 5.34 (3H, m), 5.58 (1H, d, J=8.15 Hz).

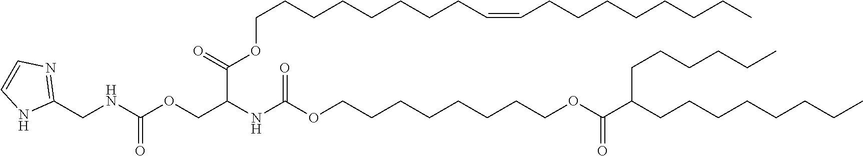 [SMILES: CCCCCCCC/C=C\CCCCCCCCOC(=O)C(COC(=O)NCc1ncc[nH]1)NC(=O)OCCCCCCCCOC(=O)C(CCCCCC)CCCCCCCC]

(Z)-1-(1H-imidazol-2-yl)-6-((octadec-9-en-1-yloxy)carbonyl)-3,8-dioxo-4,9-dioxa-2,7-diazaheptadecan-17-yl 2-hexyldecanoate was synthesized according to representative synthetic scheme 2 and the corresponding methods. MS [ESI]: m/z: [M+H]. calc. 889.7 obs. 890.5; ¹H NMR (400 MHz, CDCl₃): δ 0.87 (9H, m), 1.25 (52H, m), 1.60 (8H, m), 2.02 (4H, m), 2.30 (1H, m), 4.06 (4H, t, J=6.63 Hz), 4.16 (2H, m), 4.24-4.60 (4H, m), 4.65 (1H, m), 5.34 (2H, m), 5.57 (1H, d, J=8.78 Hz), 5.86 (1H, m), 7.02 (2H, s).

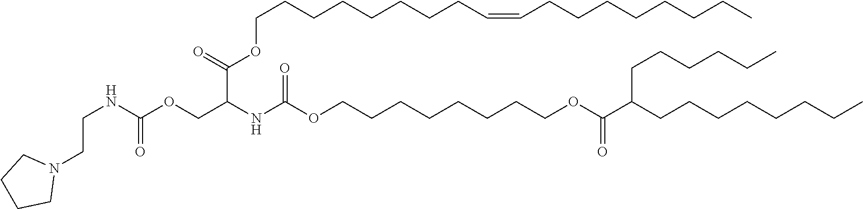 [SMILES: CCCCCCCC/C=C\CCCCCCCCOC(=O)C(COC(=O)NCCN1CCCC1)NC(=O)OCCCCCCCCOC(=O)C(CCCCCC)CCCCCCCC]

(Z)-7-((octadec-9-en-1-yloxy)carbonyl)-4,9-dioxo-1-(pyrrolidin-1-yl)-5,10-dioxa-3,8-diazaoctadecan-18-yl 2-hexyldecanoate was synthesized according to representative synthetic scheme 2 and the corresponding methods. MS [ESI]: m/z: [M+H]. calc. 906.7 obs. 907.4; ¹H NMR (400 MHz, CDCl₃): δ 0.87 (9H, m), 1.25 (52H, m), 1.61 (12H, m), 2.01 (4H, q, J=6.77 Hz), 2.30 (1H, m), 2.74 (6H, m), 3.37 (2H, m), 4.06 (4H, t, J=6.86 Hz), 4.15 (2H, t, J=6.94 Hz), 4.35 (1H, m), 4.45 (1H, m), 4.54 (1H, m), 5.34 (3H, m), 5.64 (1H, m).

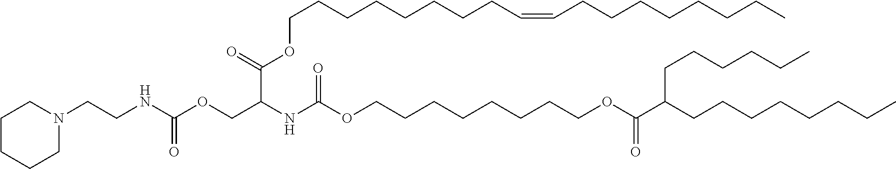 [SMILES: CCCCCCCC/C=C\CCCCCCCCOC(=O)C(COC(=O)NCCN1CCCCC1)NC(=O)OCCCCCCCCOC(=O)C(CCCCCC)CCCCCCCC]

(Z)-7-((octadec-9-en-1-yloxy)carbonyl)-4,9-dioxo-1-(piperidin-1-yl)-5,10-dioxa-3,8-diazaoctadecan-18-yl 2-hexyldecanoate was synthesized according to representative synthetic scheme 2 and the corresponding methods. MS [ESI]: m/z: [M+H]. calc. 920.8 obs. 921.6; ¹H NMR (400 MHz, CDCl₃): δ 0.87 (9H, m), 1.25 (52H, m), 1.63 (14H, m), 2.02 (4H, m), 2.30 (1H, m), 2.53 (6H, m), 3.33 (2H, m), 4.06 (4H, t, J=6.69 Hz), 4.15 (2H, t, J=6.84 Hz), 4.35 (1H, dd, J=11.38, 3.48 Hz), 4.44 (1H, dd, J=11.38, 4.03 Hz), 4.55 (1H, m), 5.34 (3H, m), 5.61 (1H, m).

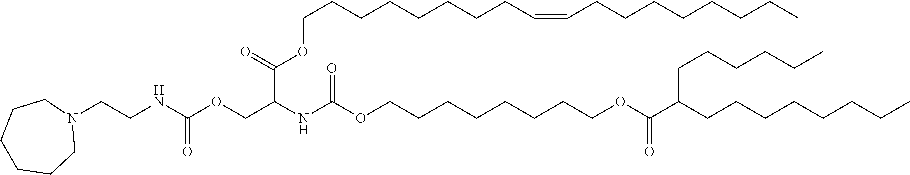 [SMILES: CCCCCCCC/C=C\CCCCCCCCOC(=O)C(COC(=O)NCCN1CCCCCC1)NC(=O)OCCCCCCCCOC(=O)C(CCCCCC)CCCCCCCC]

(Z)-1-(azepan-1-yl)-7-((octadec-9-en-1-yloxy)carbonyl)-4,9-dioxo-5,10-dioxa-3,8-diazaoctadecan-18-yl 2-hexyldecanoate was synthesized according to representative synthetic scheme 2 and the corresponding methods. MS [ESI]: m/z: [M+H]. calc. 934.8 obs. 935.6; ¹H NMR (400 MHz, CDCl₃): δ 0.87 (9H, m), 1.25 (52H, m), 1.61 (16H, m), 2.02 (4H, m), 2.30 (1H, m), 2.64 (6H, m), 3.23 (2H, m), 4.06 (4H, t, J=6.74 Hz), 4.15 (2H, t, J=6.89 Hz), 4.34 (1H, dd, J=11.52, 3.50 Hz), 4.44 (1H, dd, J=11.52, 3.86 Hz), 4.54 (1H, m), 5.34 (3H, m), 5.57 (1H, m).

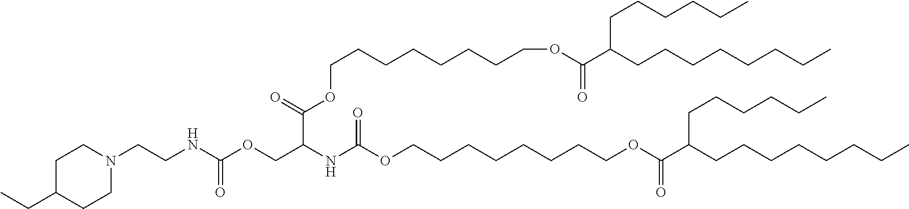

11-((((2-(4-ethylpiperidin-1-yl)ethyl)carbamoyl)oxy)methyl)-25-hexyl-10,13,24-trioxo-9,14,23-trioxa-12-azatritriacontyl 2-hexyldecanoate was synthesized according to representative synthetic scheme 2 and the corresponding methods. MS [ESI]: m/z: [M+H]. calc. 1064.9 obs. 1065.5; ¹H NMR (400 MHz, CDCl₃): δ 0.89 (15H, m), 1.27 (62H, m), 1.63 (17H, m), 2.31 (4H, ddd, J=9.05, 5.55, 4.87 Hz), 2.51-2.75 (2H, m), 2.95-3.21 (2H, m), 3.39 (2H, m), 4.08 (6H, m), 4.17 (2H, t, J=6.53 Hz), 4.30-4.62 (3H, m), 5.53-5.76 (1H, m).

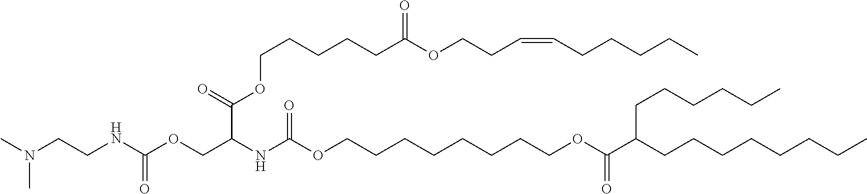 [SMILES: CCCCC/C=C\CCOC(=O)CCCCCOC(=O)C(COC(=O)NCCN(C)C)NC(=O)OCCCCCCCCOC(=O)C(CCCCCC)CCCCCCCC]

(Z)-2-methyl-9-(((6-(non-3-en-1-yloxy)-6-oxohexyl)oxy)carbonyl)-6,11-dioxo-7,12-dioxa-2,5,10-triazaicosan-20-yl 2-hexyldecanoate was synthesized according to representative synthetic scheme 2 and the corresponding methods. MS [ESI]: m/z: [M+H]. calc. 868.7 obs. 869.4; ¹H NMR (400 MHz, CDCl₃): δ 0.87 (9H, m), 1.25 (38H, m), 1.62 (10H, m), 2.03 (2H, q, J=7.31 Hz), 2.35 (11H, m), 2.55 (2H, m), 3.31 (2H, m), 4.06 (6H, m), 4.16 (2H, td, J=6.57, 1.88 Hz), 4.36 (1H, m), 4.44 (1H, dd, J=11.19, 3.57 Hz), 4.54 (1H, m), 5.33 (1H, m), 5.50 (1H, m), 5.64 (2H, m).

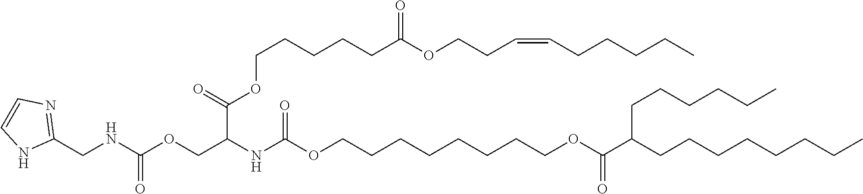 [SMILES: CCCCC/C=C\CCOC(=O)CCCCCOC(=O)C(COC(=O)NCc1ncc[nH]1)NC(=O)OCCCCCCCCOC(=O)C(CCCCCC)CCCCCCCC]

(Z)-12-(((((1H-imidazol-2-yl)methyl)carbamoyl)oxy)methyl)-10,13,20-trioxo-9,14,21-trioxa-11-azatriacont-24-en-1-yl 2-hexyldecanoate was synthesized according to representative synthetic scheme 2 and the corresponding methods. MS [ESI]: m/z: [M+H]. calc. 877.6 ohs. 878.4; ¹H NMR (400 MHz, CDCl₃): δ 0.87 (9H, m), 1.25 (38H, m), 1.61 (10H, m), 2.03 (2H, q, J=7.27 Hz), 2.30 (3H, m), 2.37 (2H, q, J=7.09 Hz), 4.06 (6H, m), 4.16 (2H, m), 4.42 (4H, m), 4.63 (1H, m), 5.33 (1H, m), 5.50 (1H, m), 5.59 (1H, d, J=7.85 Hz), 5.96 (1H, m), 7.00 (2H, s).

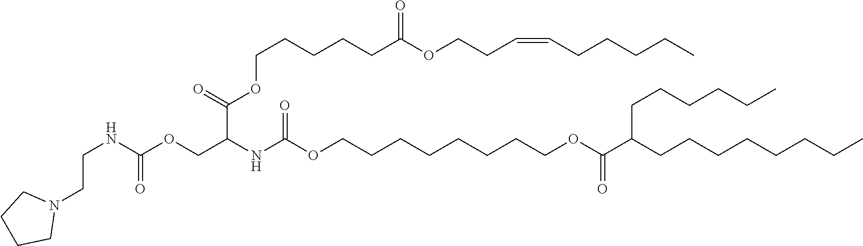 [SMILES: CCCCC/C=C\CCOC(=O)CCCCCOC(=O)C(COC(=O)NCCN1CCCC1)NC(=O)OCCCCCCCCOC(=O)C(CCCCCC)CCCCCCCC]

(Z)-10,13,20-trioxo-12-((((2-(pyrrolidin-1-yl)ethyl)carbamoyl)oxy)methyl)-9,14,21-trioxa-11-azatriacont-24-en-1-yl 2-hexyldecanoate was synthesized according to representative synthetic scheme 2 and the corresponding methods. MS [ESI]: m/z: [M+H]. calc. 894.7 obs. 895.3; ¹H NMR (400 MHz, CDCl₃): δ 0.87 (9H, m), 1.25 (38H, m), 1.61 (14H, m), 2.03 (2H, q, J=7.21 Hz), 2.30 (3H, m), 2.37 (2H, q, J=6.95 Hz), 2.64 (6H, m), 3.32 (2H, m), 4.06 (6H, m), 4.16 (2H, t, J=6.68 Hz), 4.34 (1H, dd, J=11.66, 3.34 Hz), 4.44 (1H, dd, J=11.66, 3.60 Hz), 4.53 (1H, m), 5.33 (1H, m), 5.50 (1H, m), 5.63 (2H, m).

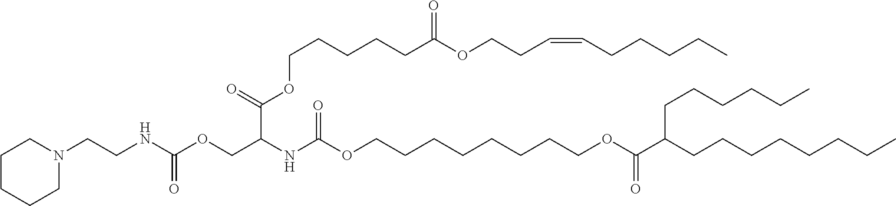 [SMILES: CCCCC/C=C\CCOC(=O)CCCCCOC(=O)C(COC(=O)NCCN1CCCCC1)NC(=O)OCCCCCCCCOC(=O)C(CCCCCC)CCCCCCCC]

(Z)-10,13,20-trioxo-12-((((2-(piperidin-1-yl)ethyl)carbamoyl)oxy)methyl)-9,14,21-trioxa-11-azatriacont-24-en-1-yl 2-hexyldecanoate was synthesized according to representative synthetic scheme 2 and the corresponding methods. MS [ESI]: m/z: [M+H]. calc. 908.7 obs. 909.1; ¹H NMR (400 MHz, CDCl₃): δ 0.88 (9H, m), 1.25 (38H, m), 1.61 (16H, m), 2.03 (2H, q, J=7.23 Hz), 2.30 (3H, m), 2.44 (8H, m), 3.28 (2H, m), 4.06 (6H, m), 4.17 (2H, t, J=6.82 Hz), 4.34 (1H, dd, J=11.46, 3.13 Hz), 4.45 (1H, dd, J=11.46, 3.77 Hz), 4.54 (1H, m), 5.33 (1H, m), 5.50 (2H, m), 5.61 (1H, d, J=8.01 Hz).

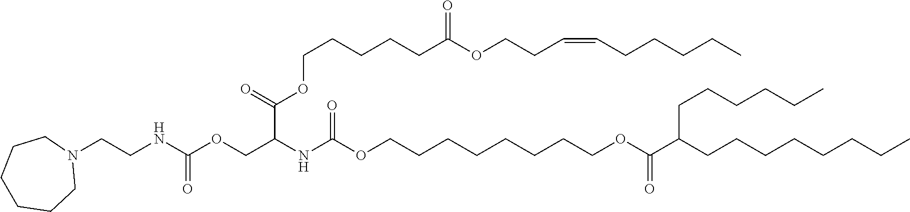 [SMILES: CCCCC/C=C\CCOC(=O)CCCCCOC(=O)C(COC(=O)NCCN1CCCCCC1)NC(=O)OCCCCCCCCOC(=O)C(CCCCCC)CCCCCCCC]

(Z)-12-((((2-(azepan-1-yl)ethyl)carbamoyl)oxy)methyl)-10,13,20-trioxo-9,14,21-trioxa-11-azatriacont-24-en-1-yl 2-hexyldecanoate was synthesized according to representative synthetic scheme 2 and the corresponding methods. MS [ESI]: m/z: [M+H]. calc. 922.7 obs. 922.9; ¹H NMR (400 MHz, CDCl₃): δ 0.86 (9H, m), 1.23 (38H, m), 1.60 (18H, m), 2.01 (2H, m), 2.28 (3H, m), 2.35 (2H, q, J=7.02 Hz), 2.69 (6H, m), 3.24 (2H, m), 4.04 (6H, m), 4.14 (2H, t, J=6.45 Hz), 4.32 (1H, m), 4.42 (1H, m), 4.52 (1H, m), 5.31 (1H, m), 5.48 (1H, m), 5.65 (2H, m).

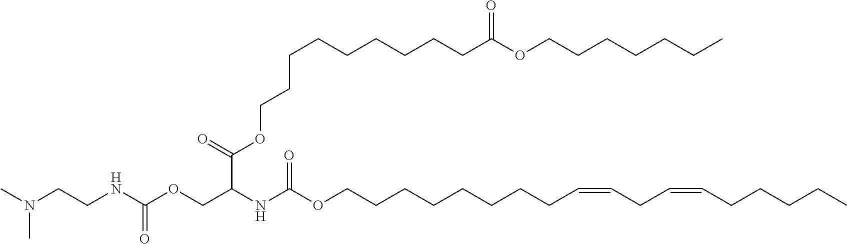 [SMILES: CCCCC/C=C\C/C=C\CCCCCCCCOC(=O)NC(COC(=O)NCCN(C)C)C(=O)OCCCCCCCCCC(=O)OCCCCCCC]

heptyl 10-((O-((2-(dimethylamino)ethyl)carbamoyl)-N-((((9Z,12Z)-octadeca-9,12-dien-1-yl)oxy)carbonyl)seryl)oxy)decanoate was synthesized according to representative synthetic scheme 2 and the corresponding methods. MS [ESI]: m/z: [M+H]. calc. 780.6 obs. 780.9; ¹H NMR (400 MHz, CDCl₃): δ 0.88 (6H, m), 1.29 (34H, m), 1.61 (8H, m), 2.04 (4H, q, J=7.34 Hz), 2.23 (6H, s), 2.28 (2H, t, J=7.94 Hz), 2.41 (2H, dd, J=7.05, 6.15 Hz), 2.77 (2H, t, J=6.57 Hz), 3.24 (2H, m), 4.05 (4H, m), 4.15 (2H, t, J=6.70 Hz), 4.29-4.47 (2H, m), 4.49-4.58 (1H, m), 5.35 (5H, m), 5.55 (1H, d, J=8.28 Hz).

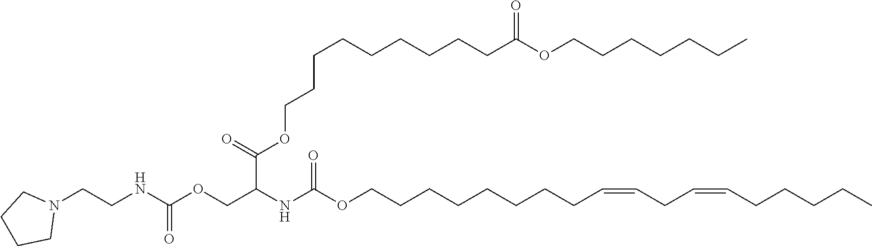 [SMILES: CCCCC/C=C\C/C=C\CCCCCCCCOC(=O)NC(COC(=O)NCCN1CCCC1)C(=O)OCCCCCCCCCC(=O)OCCCCCCC]

heptyl 10-((N-((((9Z,12Z)-octadeca-9,12-dien-1-yl)oxy)carbonyl)-O-((2-(pyrrolidin-1-yl)ethyl)carbamoyl)seryl)oxy)decanoate was synthesized according to representative synthetic scheme 2 and the corresponding methods. MS [ESI]: m/z: [M+H]. calc. 806.6 obs. 807.2; ¹H NMR (400 MHz, CDCl₃): δ 0.88 (6H, t, J=6.62 Hz), 1.29 (32H, m), 1.61 (14H, m), 2.05 (4H, q, J=6.89 Hz), 2.28 (2H, t, J=7.86 Hz), 2.50 (4H, m), 2.57 (2H, dd, J=7.30, 6.07 Hz), 2.77 (2H, t, J=6.41 Hz), 3.26 (2H, m), 4.05 (4H, m), 4.15 (2H, t, J=6.75 Hz), 4.29-4.48 (2H, m), 4.53 (1H, m), 5.35 (5H, m), 5.55 (1H, d, J=8.20 Hz).

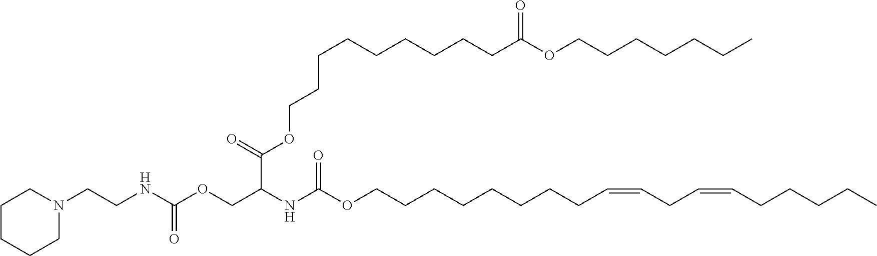 [SMILES: CCCCC/C=C\C/C=C\CCCCCCCCOC(=O)NC(COC(=O)NCCN1CCCCC1)C(=O)OCCCCCCCCCC(=O)OCCCCCCC]

heptyl 10-((N-((((9Z,12Z)-octadeca-9,12-dien-1-yl)oxy)carbonyl)-O-((2-(piperidin-1-yl)ethyl)carbamoyl)seryl)oxy)decanoate was synthesized according to representative synthetic scheme 2 and the corresponding methods. MS [ESI]: m/z: [M+H]. calc. 820.6 obs. 821.1; ¹H NMR (400 MHz, CDCl₃): δ 0.88 (6H, t, J=6.41 Hz), 1.29 (32H, m), 1.40-1.49 (2H, m), 1.50-1.73 (14H, m), 2.05 (4H, q, J=7.20 Hz), 2.28 (211, t, J=7.94 Hz), 2.31-2.45 (6H, m), 2.77 (2H, t, J=6.41 Hz), 3.24 (2H, m), 4.05 (4H, m), 4.15 (2H, t, J=6.75 Hz), 4.29-4.49 (2H, m), 4.56 (1H, m), 5.35 (5H, m), 5.55 (1H, d, J=8.46 Hz).

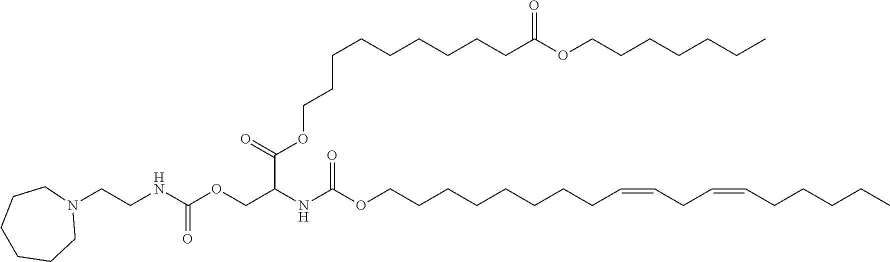 [SMILES: CCCCC/C=C\C/C=C\CCCCCCCCOC(=O)NC(COC(=O)NCCN1CCCCCC1)C(=O)OCCCCCCCCCC(=O)OCCCCCCC]

heptyl 10-((O-((2-(azepan-1-yl)ethyl)carbamoyl)-N-((((9Z,12Z)-octadeca-9,12-dien-1-yl)oxy)carbonyl)seryl)oxy)decanoate was synthesized according to representative synthetic scheme 2 and the corresponding methods. MS [ESI]: m/z: [M+H]. calc. 834.6 obs. 835.3; ¹H NMR (400 MHz, CDCl₃): δ 0.88 (6H, m), 1.29 (32H, m), 1.59 (18H, m), 2.04 (4H, q, J=6.78 Hz), 2.28 (2H, t, J=7.94 Hz), 2.62 (6H, m), 2.77 (2H, t, J=6.28 Hz), 3.20 (2H, m), 4.05 (4H, m), 4.15 (2H, t, J=6.53 Hz), 4.30-4.49 (2H, m), 4.50-4.60 (1H, m), 5.35 (5H, m), 5.55 (1H, d, J=8.03 Hz).

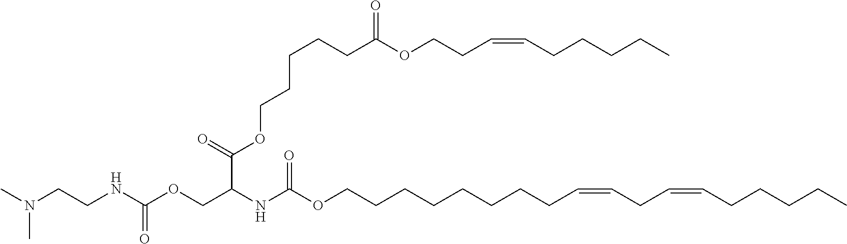 [SMILES: CCCCC/C=C\C/C=C\CCCCCCCCOC(=O)NC(COC(=O)NCCN(C)C)C(=O)OCCCCCC(=O)OCC/C=C\CCCCC]

(Z)-non-3-en-1-yl 6-((O- ((2-(dimethylamino)ethyl)carbamoyl)-N-((((9Z,12Z)-octadeca-9,12-dien-1-yl)oxy)carbonyl)seryl)oxy)hexanoate was synthesized according to representative synthetic scheme 2 and the corresponding methods. MS [ESI]: m/z: [M+H]. calc. 750.6 obs. 750.9; ¹H NMR (400 MHZ, CDC13): δ 0.89 (6H, t, J=6.62 Hz), 1.30 (24H, m), 1.55-1.71 (6H, m), 2.04 (6H, i, J=7.52 Hz), 2.22 (6H, s), 2.30 (2H, t, J=7.68 Hz), 2.38 (4H, m), 2.77 (2H, t, J=6.58 Hz), 3.24 (2H, m), 4.06 (4H, t, J=6.79 Hz), 4.16 (2H, t, J=6.70 Hz), 4.31-4.48 (2H, m), 4.50-4.58 (1H, m), 5.34 (6H, m), 5.44-5.61 (2H, m).

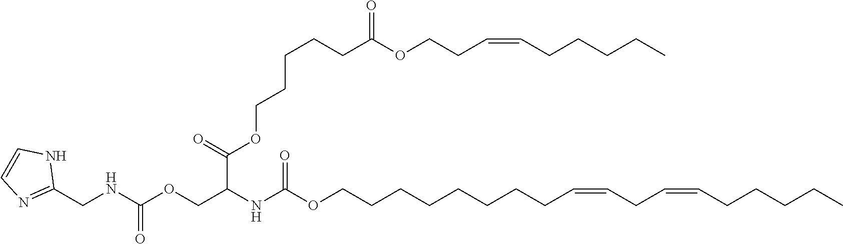 [SMILES: CCCCC/C=C\C/C=C\CCCCCCCCOC(=O)NC(COC(=O)NCc1ncc[nH]1)C(=O)OCCCCCC(=O)OCC/C=C\CCCCC]

(Z)-non-3-en-1-yl 6-((O-(((1H-imidazol-2-yl)methyl)carbamoyl)-N-((((9Z,12Z)-octadeca-9,12-dien-1-yl)oxy)carbonyl)seryl)oxy)hexanoate was synthesized according to representative synthetic scheme 2 and the corresponding methods. MS [ESI]: m/z: [M+H]. calc. 759.5 obs. 759.9; ¹H NMR (400 MHz, CDCl₃): δ 0.89 (6H, t, J=6.62 Hz), 1.30 (24H, m), 1.54-1.71 (6H, m), 2.04 (6H, i, J=7.12 Hz), 2.30 (2H, t, J=7.53 Hz), 2.37 (2H, q, J=6.99 Hz), 2.77 (2H, t, J=6.41 Hz), 4.06 (4H, t, J=6.89 Hz), 4.11-4.24 (2H, m), 4.30-4.52 (411, m), 4.58-4.68 (1H, m), 5.34 (6H, m), 5.45-5.58 (2H, m), 5.66-5.75 (1H, m), 6.97 (2H, s).

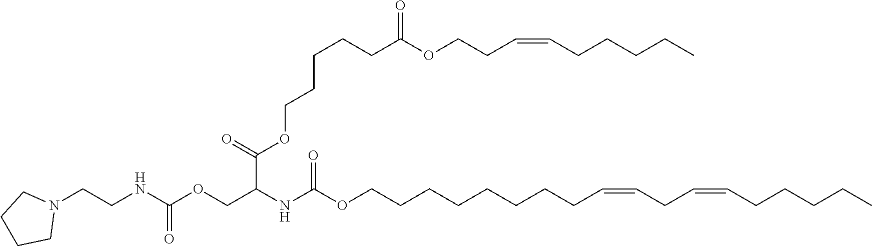 [SMILES: CCCCC/C=C\C/C=C\CCCCCCCCOC(=O)NC(COC(=O)NCCN1CCCC1)C(=O)OCCCCCC(=O)OCC/C=C\CCCCC]

(Z)-non-3-en-1-yl 6-((N-((((9Z,12Z)-octadeca-9,12-dien-1-yl)oxy)carbonyl)-O-((2-(pyrrolidin-1-yl)ethyl)carbamoyl)seryl)oxy)hexanoate was synthesized according to representative synthetic scheme 2 and the corresponding methods. MS [ESI]: m/z: [M+H]. calc. 776.6 obs. 777.3; ¹H NMR (400 MHz, CDCl₃): δ 0.89 (6H, t, J=6.49 Hz), 1.30 (24H, m), 1.78 (10H, m), 2.04 (6H, i, J=7.86 Hz), 2.30 (2H, t, J=7.48 Hz), 2.37 (2H, q, J=7.34 Hz), 2.51 (4H, m), 2.58 (2H, dd, J=7.21, 6.22 Hz), 2.77 (2H, dd, J=7.35, 6.56 Hz), 3.27 (2H, m), 4.06 (4H, t, J=6.83 Hz), 4.16 (2H, t, J=6.66 Hz), 4.39 (2H, m), 4.54 (1H, m), 5.35 (6H, m), 5.45-5.55 (1H, m), 5.59 (1H, d, J=8.73 Hz).

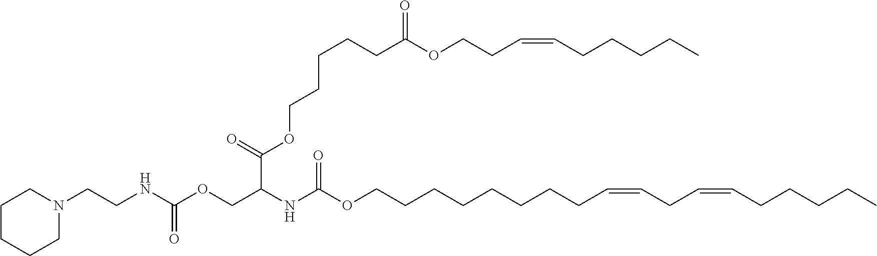 [SMILES: CCCCC/C=C\C/C=C\CCCCCCCCOC(=O)NC(COC(=O)NCCN1CCCCC1)C(=O)OCCCCCC(=O)OCC/C=C\CCCCC]

(Z)-non-3-en-1-yl 6-((N-((((9Z,12Z)-octadeca-9,12-dien-1-yl)oxy)carbonyl)-O-((2-(piperidin-1-yl)ethyl)carbamoyl)seryl)oxy)hexanoate was synthesized according to representative synthetic scheme 2 and the corresponding methods. MS [ESI]: m/z: [M+H]. calc. 790.6 obs. 791.2; ¹H NMR (400 MHz, CDCl₃): δ 0.89 (6H, t, J=6.58 Hz), 1.30 (24H, m), 1.63 (12H, m), 2.04 (6H, i, J=7.12 Hz), 2.36 (10H, m), 2.77 (2H, t, J=6.46 Hz), 3.24 (2H, m), 4.06 (4H, t, J=6.83 Hz), 4.17 (2H, t, J=6.66 Hz), 4.29-4.48 (2H, m), 4.49-4.61 (1H, m), 5.34 (6H, m), 5.44-5.61 (2H, m).

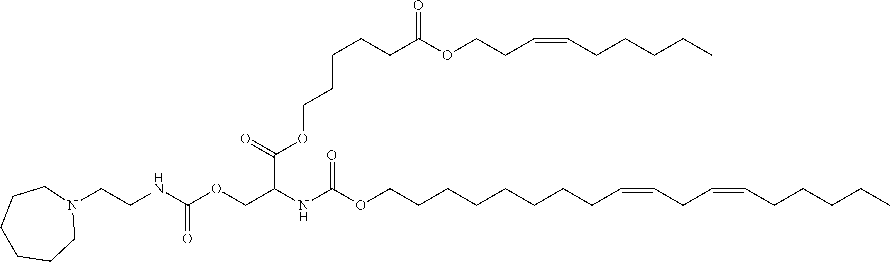 [SMILES: CCCCC/C=C\C/C=C\CCCCCCCCOC(=O)NC(COC(=O)NCCN1CCCCCC1)C(=O)OCCCCCC(=O)OCC/C=C\CCCCC]

(Z)-non-3-en-1-yl 6-((O-((2-(azepan-1-yl)ethyl)carbamoyl)-N-((((9Z,12Z)-octadeca-9,12-dien-1-yl)oxy)carbonyl)seryl)oxy)hexanoate was synthesized according to representative synthetic scheme 2 and the corresponding methods. MS [ESI]: m/z: [M+H]. calc. 804.6 obs. 805.1; ¹H NMR (400 MHz, CDCl₃): δ 0.88 (6H, t, J=6.58 Hz), 1.30 (24H, m), 1.59 (14H, m), 2.03 (6H, i, J=6.58 Hz), 2.30 (2H, t, J=7.42 Hz), 2.36 (2H, q, J=7.43 Hz), 2.53-2.68 (6H, m), 2.77 (2H, t, J=6.32 Hz), 3.20 (2H, m), 4.06 (4H, t, J=6.92 Hz), 4.16 (2H, t, J=6.62 Hz), 4.29-4.47 (2H, m), 4.48-4.59 (1H, m), 5.27-5.43 (6H, m), 5.45-5.54 (1H, m), 5.54-5.63 (1H, m).

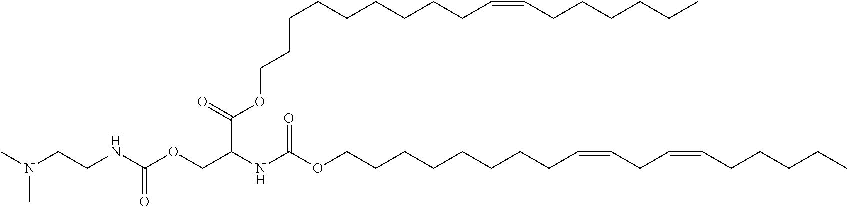 [SMILES: CCCCC/C=C\C/C=C\CCCCCCCCOC(=O)NC(COC(=O)NCCN(C)C)C(=O)OCCCCCCCCC/C=C\CCCCCC]

(9Z,12Z)-octadeca-9,12-dien-1-yl O-((2-(dimethylamino)ethyl)carbamoyl)-N-((((9Z,12Z)-octadeca-9,12-dien-1-yl)oxy)carbonyl)serinate was synthesized according to representative synthetic scheme 2 and the corresponding methods. MS [ESI]: m/z: [M+H]. calc. 760.6 obs. 760.9; ¹H NMR (400 MHz, CDCl₃): δ 0.89 (6H, t, J=6.83 Hz), 1.30 (32H, m), 1.62 (4H, m), 2.04 (8H, q, J=6.37 Hz), 2.35 (6H, s), 2.55 (2H, m), 2.76 (4H, t, J=6.45 Hz), 3.31 (2H, m), 4.06 (2H, t, J=6.87 Hz), 4.15 (2H, t, J=6.83 Hz), 4.29-4.49 (2H, m), 4.49-4.60 (1H, m), 5.36 (8H, m), 5.45-5.73 (2H, m).

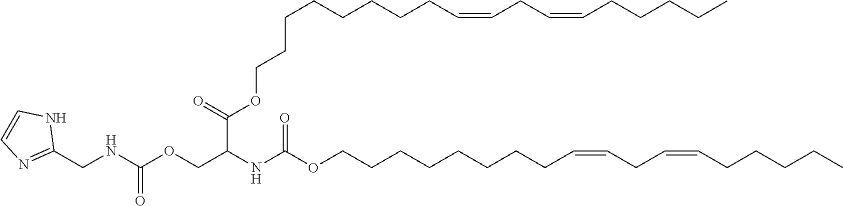 [SMILES: CCCCC/C=C\C/C=C\CCCCCCCCOC(=O)NC(COC(=O)NCc1ncc[nH]1)C(=O)OCCCCCCCC/C=C\C/C=C\CCCCC]

(9Z,12Z)-octadeca-9,12-dien-1-yl O-(((1H-imidazol-2-yl)methyl)carbamoyl)-N-((((9Z,12Z)-octadeca-9,12-dien-1-yl)oxy)carbonyl)serinate was synthesized according to representative synthetic scheme 2 and the corresponding methods. MS [ESI]: m/z: [M+H]. calc. 769.6 obs. 769.9; ¹H NMR (400 MHz, CDCl₃): δ 0.89 (6H, t, J=6.66 Hz), 1.30 (32H, m), 1.62 (4H, m), 2.04 (8H, q, J=7.28 Hz), 2.76 (4H, tm, J=6.49, 1.94 Hz), 3.91-4.22 (5H, m), 4.22-4.34 (1H, m), 4.39-4.58 (3H, m), 4.58-4.73 (2H, m), 5.35 (7H, m), 5.57 (1H, d, J=8.84 Hz), 5.86-6.01 (1H, m), 7.03 (2H, m).

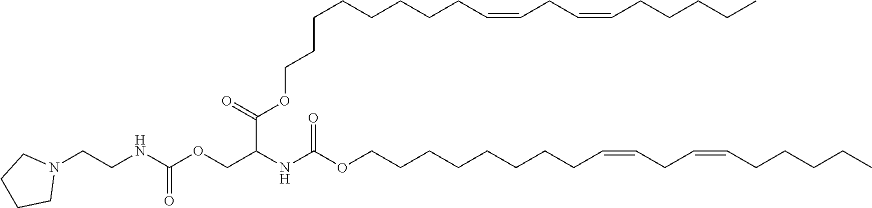 [SMILES: CCCCC/C=C\C/C=C\CCCCCCCCOC(=O)NC(COC(=O)NCCN1CCCC1)C(=O)OCCCCCCCC/C=C\C/C=C\CCCCC]

(9Z,12Z)-octadeca-9,12-dien-1-yl N-((((9Z,12Z)-octadeca-9,12-dien-1-yl)oxy)carbonyl)-O-((2-(pyrrolidin-1-yl)ethyl)carbamoyl)serinate was synthesized according to representative synthetic scheme 2 and the corresponding methods. MS [ESI]: m/z: [M+H]. calc. 786.6 obs. 787.2; ¹H NMR (400 MHz, CDCl₃): δ 0.89 (6H, t, J=6.58, Hz), 1.30 (32H, m), 1.61 (4H, m), 1.84 (4H, m), 2.04 (8H, q, J=7.11 Hz), 2.55-2.73 (6H, m), 2.76 (4H, t, J=6.58 Hz), 3.32 (2H, m), 4.05 (2H, t, J=7.31 Hz), 4.15 (2H, t, J=7.08 Hz), 4.30-4.48 (2H, m), 4.50-4.60 (1H, m), 5.35 (8H, m), 5.55-5.64 (1H, m).

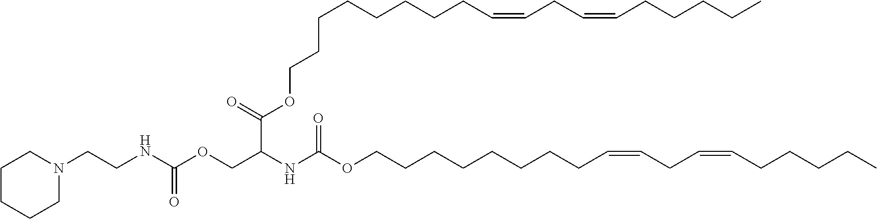 [SMILES: CCCCC/C=C\C/C=C\CCCCCCCCOC(=O)NC(COC(=O)NCCN1CCCCC1)C(=O)OCCCCCCCC/C=C\C/C=C\CCCCC]

(9Z,12Z)-octadeca-9,12-dien-1-yl N-((((9Z,12Z)-octadeca-9,12-dien-1-yl)oxy)carbonyl)-O-((2-(piperidin-1-yl)ethyl)carbamoyl)serinate was synthesized according to representative synthetic scheme 2 and the corresponding methods. MS [ESI]: m/z: [M+H]. calc. 800.7 obs. 801.4; ¹H NMR (400 MHz, CDCl₃): δ 0.89 (6H, t, J=6.70 Hz), 1.30 (32H, m), 1.41-1.51 (2H, m), 1.62 (8H, m), 2.05 (8H, q, J=6.90 Hz), 2.45 (6H, m), 2.77 (4H, t, J=6.54 Hz), 3.22-3.35 (2H, m), 4.06 (2H, t, J=6.89 Hz), 4.16 (2H, t, J=6.73 Hz), 4.31-4.49 (2H, m), 4.49-4.60 (1H, m), 5.35 (8H, m), 5.57 (1H, d, J=8.55 Hz).

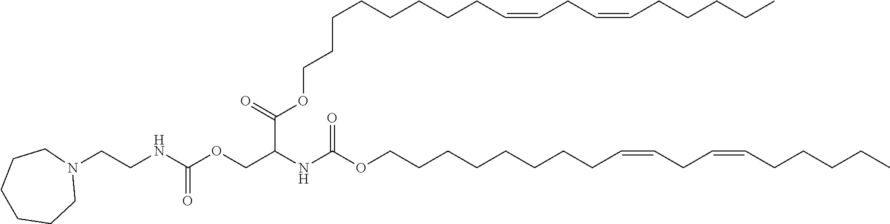 [SMILES: CCCCC/C=C\C/C=C\CCCCCCCCOC(=O)NC(COC(=O)NCCN1CCCCCC1)C(=O)OCCCCCCCC/C=C\C/C=C\CCCCC]

(9Z,12Z)-octadeca-9,12-dien-1-yl O-((2-(azepan-1-yl)ethyl)carbamoyl)-N-((((9Z,12Z)-octadeca-9,12-dien-1-yl)oxy)carbonyl)serinate was synthesized according to representative synthetic scheme 2 and the corresponding methods. MS [ESI]: m/z: [M+H]. calc. 814.7 obs. 815.1; ¹H NMR (400 MHz, CDCl₃): δ 0.89 (6H, t, J=6.66, Hz), 1.30 (32H, m), 1.61 (12H, m), 2.05 (8H, q, J=7.28 Hz), 2.54-2.74 (6H, m), 2.77 (4H, t, J=6.70 Hz), 3.26 (2H, m), 4.06 (2H, t, J=7.17 Hz), 4.15 (2H, t, J=6.62 Hz), 4.29-4.48 (2H, m), 4.55 (1H, m), 5.35 (8H, m), 5.55 (1H, d, J=8.70 Hz).

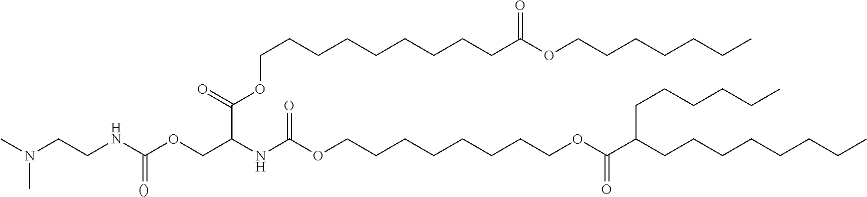

heptyl 10-((O-((2-(dimethylamino)ethyl)carbamoyl)-N-(((8-((2-hexyldecanoyl)oxy)octyl)oxy)carbonyl)seryl)oxy)decanoate was synthesized according to representative synthetic scheme 2 and the corresponding methods. MS [ESI]: m/z: [M+H]. calc. 898.7 obs. 899.2; ¹H NMR (400 MHz, CDCl₃): δ 0.87 (9H, m), 1.25 (48H, m), 1.60 (12H, m), 2.28 (9H, m), 2.47 (2H, t, J=6.32 Hz), 3.27 (2H, m), 4.06 (6H, m), 4.15 (2H, t, J=6.88 Hz), 4.35 (1H, dd, J=11.46, 3.53 Hz), 4.43 (1H, dd, J=11.46, 3.79 Hz), 4.54 (1H, m), 5.42 (1H, m), 5.59 (1H, d, J=8.64 Hz).

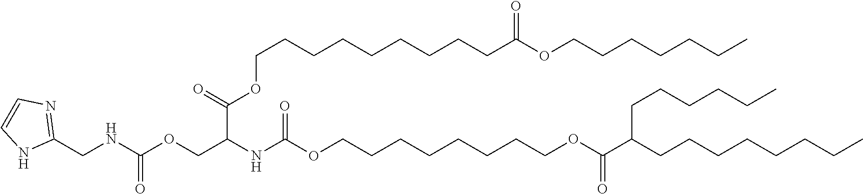 [SMILES: CCCCCCCCC(CCCCCC)C(=O)OCCCCCCCCOC(=O)NC(COC(=O)NCc1ncc[nH]1)C(=O)OCCCCCCCCCC(=O)OCCCCCCC]

21-(((((1H-imidazol-2-yl)methyl)carbamoyl)oxy)methyl)-9,20,23-trioxo-8,19,24-trioxa-22-azadotriacontan-32-yl 2-hexyldecanoate was synthesized according to representative synthetic scheme 2 and the corresponding methods. MS [ESI]: m/z: [M+H]. calc. 907.7 obs. 908.4; ¹H NMR (400 MHz, CDCl₃): δ 0.87 (9H, m), 1.25 (48H, m), 1.61 (12H, m), 2.29 (3H, m), 4.06 (6H, m), 4.15 (2H, t, J=6.84 Hz), 4.56 (5H, m), 5.62 (1H, d, J=8.53 Hz), 6.11 (1H, m), 7.06 (2H, s).

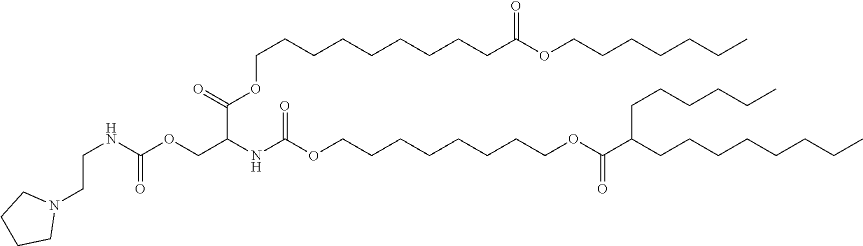 [SMILES: CCCCCCCCC(CCCCCC)C(=O)OCCCCCCCCOC(=O)NC(COC(=O)NCCN1CCCC1)C(=O)OCCCCCCCCCC(=O)OCCCCCCC]

heptyl 10-((N-(((8-((2-hexyldecanoyl)oxy)octyl)oxy)carbonyl)-O-((2-(pyrrolidin-1-yl)ethyl)carbamoyl)seryl)oxy)decanoate was synthesized according to representative synthetic scheme 2 and the corresponding methods. MS [ESI]: m/z: [M+H]. calc. 924.7 obs. 925.3; ¹H NMR (400 MHz, CDCl₃): δ 0.87 (9H, m), 1.25 (48H, m), 1.61 (16H, m), 2.28 (3H, m), 2.58 (6H, m), 3.30 (2H, m), 4.05 (6H, m), 4.15 (2H, t, J=6.87 Hz), 4.34 (1H, dd, J=11.45, 3.28 Hz), 4.44 (1H, dd, J=11.45, 4.00 Hz), 4.54 (1H, m), 5.46 (1H, m), 5.59 (1H, d, J=8.16 Hz).

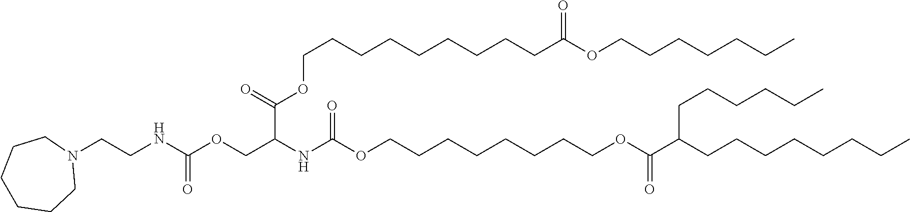 [SMILES: CCCCCCCCC(CCCCCC)C(=O)OCCCCCCCCOC(=O)NC(COC(=O)NCCN1CCCCCC1)C(=O)OCCCCCCCCCC(=O)OCCCCCCC]

21-((((2-(azepan-1-yl)ethyl)carbamoyl)oxy)methyl)-9,20,23-trioxo-8,19,24-trioxa-22-azadotriacontan-32-yl 2-hexyldecanoate was synthesized according to representative synthetic scheme 2 and the corresponding methods. MS [ESI]: m/z: [M+H]. calc. 952.8 ohs. 953.6; ¹H NMR (400 MHz, CDCl₃): δ 0.87 (9H, m), 1.25 (48H, m), 1.61 (20H, m), 2.28 (3H, m), 2.67 (6H, m), 3.25 (2H, m), 4.05 (6H, m), 4.15 (2H, t, J=6.83 Hz), 4.34 (1H, dd, J=11.49, 3.04 Hz), 4.44 (1H, dd, J=11.49, 5.08 Hz), 4.55 (1H, m), 5.31-5.62 (2H, m).

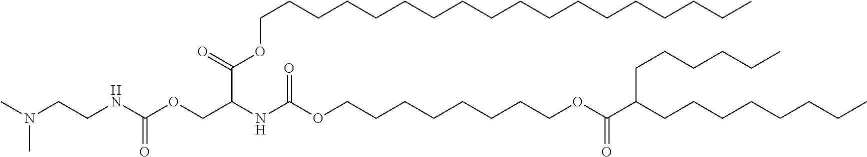 [SMILES: CCCCCCCCCCCCCCCCCCOC(=O)C(COC(=O)NCCN(C)C)NC(=O)OCCCCCCCCOC(=O)C(CCCCCC)CCCCCCCC]

2-methyl-9-((octadecyloxy)carbonyl)-6,11-dioxo-7,12-dioxa-2,5,10-triazaicosan-20-yl 2-hexyldecanoate was synthesized according to representative synthetic scheme 2 and the corresponding methods. MS [ESI]: m/z: [M+H]. calc. 882.8 obs. 883.0; ¹H NMR (400 MHz, CDCl₃): δ 0.88 (9H, m), 1.26 (44H, m), 1.58 (16H, m), 2.22 (6H, s), 2.30 (4H, t, J=7.86 Hz), 2.35-2.43 (1H, m), 3.24 (2H, m), 4.06 (6H, td, J=6.66, 2.56 Hz), 4.17 (2H, td, J=6.58, 1.54 Hz), 4.29-4.47 (2H, m), 4.48-4.60 (1H, m), 5.25-5.36 (1H, m), 5.51-5.61 (1H, m).

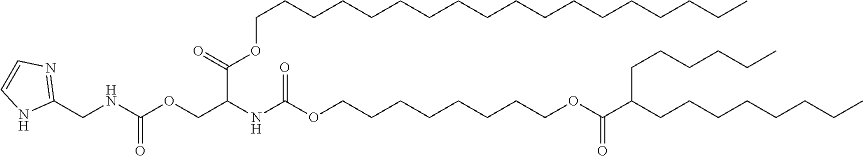 [SMILES: CCCCCCCCCCCCCCCCCCOC(=O)C(COC(=O)NCc1ncc[nH]1)NC(=O)OCCCCCCCCOC(=O)C(CCCCCC)CCCCCCCC]

1-(1H-imidazol-2-yl)-6-((octadecyloxy)carbonyl)-3,8-dioxo-4,9-dioxa-2,7-diazaheptadecan-17-yl 2-hexyldecanoate was synthesized according to representative synthetic scheme 2 and the corresponding methods. MS [ESI]: m/z: [M+H]. calc. 891.7 obs. 891.9; ¹H NMR (400 MHz, CDCl₃): δ 0.87 (9H, t, J=6.53 Hz), 1.26 (48H, m), 1.61 (12H, m), 2.30 (3H, t, J=7.09 Hz), 4.06 (8H, m), 4.30-4.51 (4H, m), 4.62 (1H, m), 5.55 (1H, d, J=7.69 Hz), 5.69-5.82 (1H, m), 6.97 (2H, s).

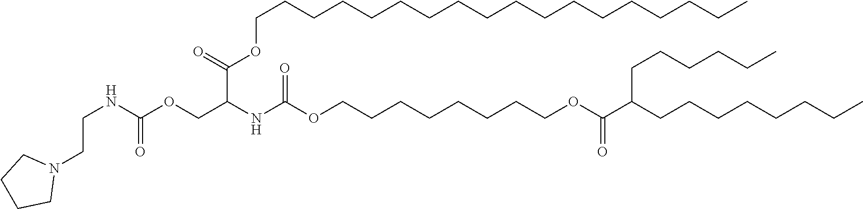 [SMILES: CCCCCCCCCCCCCCCCCCOC(=O)C(COC(=O)NCCN1CCCC1)NC(=O)OCCCCCCCCOC(=O)C(CCCCCC)CCCCCCCC]

7-((octadecyloxy)carbonyl)-4,9-dioxo-1-(pyrrolidin-1-yl)-5,10-dioxa-3,8-diazaoctadecan-18-yl 2-hexyldecanoate was synthesized according to representative synthetic scheme 2 and the corresponding methods. MS [ESI]: m/z: [M+H]. calc. 908.8 obs. 909.2; ¹H NMR (400 MHz, CDCl₃): δ 0.87 (9H, m), 1.26 (48H, m), 1.67 (12H, m), 1.77 (4H, m), 2.30 (3H, t, J=7.86 Hz), 2.51 (6H, m), 3.26 (2H, m), 4.06 (6H, td, J=6.53, 2.45 Hz), 4.16 (2H, td, J=6.45, 1.88 Hz), 4.29-4.48 (2H, m), 4.55 (1H, m), 5.36 (1H, m), 5.57 (1H, d, J=7.69 Hz).

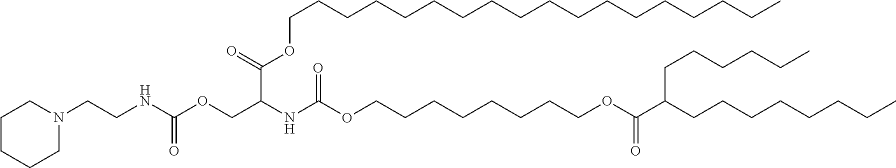 [SMILES: CCCCCCCCCCCCCCCCCCOC(=O)C(COC(=O)NCCN1CCCCC1)NC(=O)OCCCCCCCCOC(=O)C(CCCCCC)CCCCCCCC]

7-((octadecyloxy)carbonyl)-4,9-dioxo-1-(piperidin-1-yl)-5,10-dioxa-3,8-diazaoctadecan-18-yl 2-hexyldecanoate was synthesized according to representative synthetic scheme 2 and the corresponding methods. MS [ESI]: m/z: [M+H]. calc. 922.8 obs. 923.5; ¹H NMR (400 MHz, CDCl₃): δ 0.87 (9H, m), 1.26 (48H, m), 1.50-1.74 (18H, m), 2.30 (9H, m), 3.25 (2H, d, J=6.83 Hz), 4.05 (6H, td, J=6.66, 2.82 Hz), 4.17 (2H, td, J=6.49, 1.88 Hz), 4.30-4.48 (2H, m), 4.49-4.59 (1H, m), 5.36 (1H, m), 5.55-5.62 (1H, m).

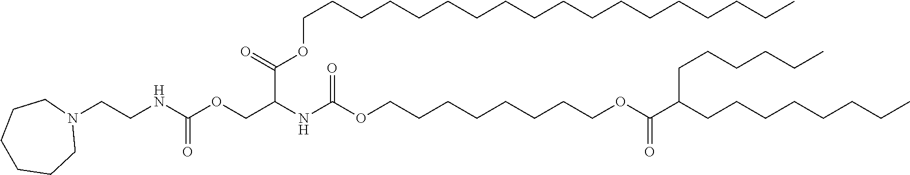 [SMILES: CCCCCCCCCCCCCCCCCCOC(=O)C(COC(=O)NCCN1CCCCCC1)NC(=O)OCCCCCCCCOC(=O)C(CCCCCC)CCCCCCCC]

1-(azepan-1-yl)-7-((octadecyloxy)carbonyl)-4,9-dioxo-5,10-dioxa-3,8-diazaoctadecan-18-yl 2-hexyldecanoate was synthesized according to representative synthetic scheme 2 and the corresponding methods. MS [ESI]: m/z: [M+H]. calc. 936.8 obs. 937.2; ¹H NMR (400 MHz, CDCl₃): δ 0.87 (9H, m), 1.26 (48H, m), 1.50-1.74 (20H, m), 2.30 (9H, m), 3.19-3.30 (2H, m), 4.05 (6H, td, J=6.66, 2.82 Hz), 4.17 (2H, td, J=6.49, 1.54 Hz), 4.30-4.48 (2H, m), 4.49-4.59 (1H, m), 5.31-5.41 (1H, m), 5.58 (1H, d, J=8.20 Hz).

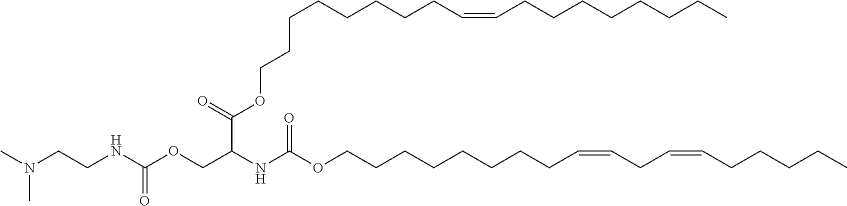 [SMILES: CCCCC/C=C\C/C=C\CCCCCCCCOC(=O)NC(COC(=O)NCCN(C)C)C(=O)OCCCCCCCC/C=C\CCCCCCCC]

(Z)-octadec-9-en-1-yl O-((2-(dimethylamino)ethyl)carbamoyl)-N-((((9Z,12Z)-octadeca-9,12-dien-1-yl)oxy)carbonyl)serinate was synthesized according to representative synthetic scheme 2 and the corresponding methods. MS [ESI]: m/z: [M+H]. calc. 762.6 obs. 762.8; ¹H NMR (400 MHz, CDCl₃): δ 0.88 (6H, m), 1.30 (38H, m), 1.62 (4H, m), 2.04 (8H, m), 2.26 (6H, s), 2.45 (2H, t, J=6.03 Hz), 2.77 (2H, t, J=6.42 Hz), 3.27 (2H, m), 4.06 (2H, t, J=7.00 Hz), 4.15 (2H, t, J=6.89 Hz), 4.34 (1H, dd, J=11.25, 2.96 Hz), 4.43 (1H, dd, J=11.25, 3.83 Hz), 4.54 (1H, m), 5.35 (6H, m), 5.57 (1H, m).

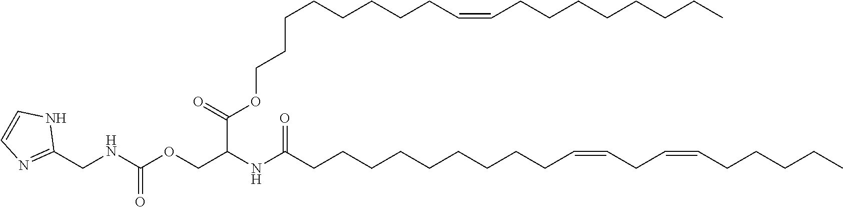 [SMILES: CCCCC/C=C\C/C=C\CCCCCCCCCC(=O)NC(COC(=O)NCc1ncc[nH]1)C(=O)OCCCCCCCC/C=C\CCCCCCCC]

(Z)-octadec-9-en-1-yl O-(((1H-imidazol-2-yl)methyl)carbamoyl)-N-((((9Z,12Z)-octadeca-9,12-dien-1-yl)oxy)carbonyl)serinate was synthesized according to representative synthetic scheme 2 and the corresponding methods. MS [ESI]: m/z: [M+H]. calc. 771.6 obs. 772.3; ¹H NMR (400 MHz, CDCl₃): δ 0.88 (6H, m), 1.30 (38H, m), 1.62 (4H, m), 2.04 (8H, m), 2.77 (2H, t, J=6.32 Hz), 4.08 (4H, m), 4.39 (4H, m), 4.65 (1H, m), 5.34 (6H, m), 5.57 (1H, m), 5.85 (1H, m), 7.01 (2H, s).

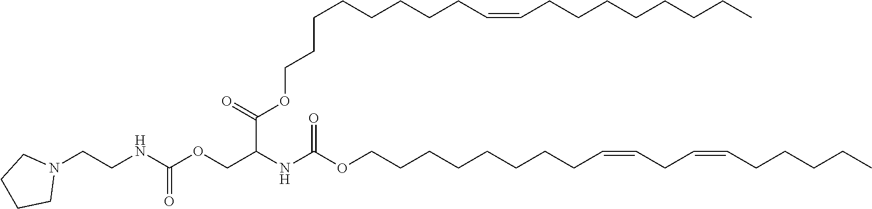 [SMILES: CCCCC/C=C\C/C=C\CCCCCCCCOC(=O)NC(COC(=O)NCCN1CCCC1)C(=O)OCCCCCCCC/C=C\CCCCCCCC]

(Z)-octadec-9-en-1-yl N-((((9Z,12Z)-octadeca-9,12-dien-1-yl)oxy)carbonyl)-O-((2-(pyrrolidin-1-yl)ethyl)carbamoyl)serinate was synthesized according to representative synthetic scheme 2 and the corresponding methods. MS [ESI]: m/z: [M+H]. calc. 788.7 obs. 789.1; ¹H NMR (400 MHz, CDCl₃): δ 0.88 (6H, m), 1.30 (38H, m), 1.62 (4H, m), 1.82 (4H, m), 2.04 (8H, m), 2.60 (6H, m), 2.77 (2H, t, J=6.36 Hz), 3.30 (2H, m), 4.06 (2H, t, J=7.26 Hz), 4.15 (2H, t, J=6.75 Hz), 4.34 (1H, dd, J=11.44, 2.96 Hz), 4.43 (1H, dd, J=11.44, 3.65 Hz), 4.54 (1H, m), 5.35 (6H, m), 5.48 (1H, m), 5.59 (1H, d, J=8.08 Hz).

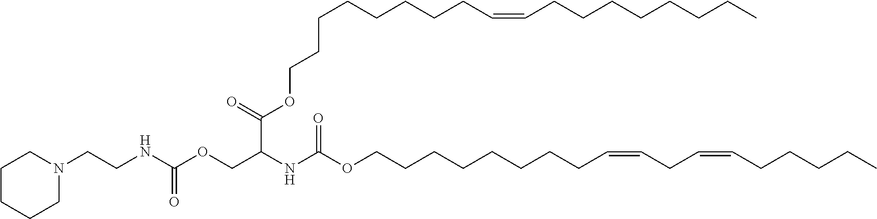 [SMILES: CCCCC/C=C\C/C=C\CCCCCCCCOC(=O)NC(COC(=O)NCCN1CCCCC1)C(=O)OCCCCCCCC/C=C\CCCCCCCC]

(Z)-octadec-9-en-1-yl N-((((9Z,12Z)-octadeca-9,12-dien-1-yl)oxy)carbonyl)-O-((2-(piperidin-1-yl)ethyl)carbamoyl)serinate was synthesized according to representative synthetic scheme 2 and the corresponding methods. MS [ESI]: m/z: [M+H]. calc. 802.7 obs. 802.9; ¹H NMR (400 MHz, CDCl₃): δ 0.88 (6H, m), 1.30 (38H, m), 1.46 (2H, m), 1.62 (8H, m), 2.04 (8H, m), 2.43 (6H, m), 2.76 (2H, t, J=6.45 Hz), 3.27 (2H, m), 4.07 (2H, t, J=7.09 Hz), 4.16 (2H, t, J=6.81 Hz), 4.34 (1H, dd, J=11.61, 2.52 Hz), 4.44 (1H, dd, J=11.61, 3.50 Hz), 4.55 (1H, m), 5.34 (6H, m), 5.56 (1H, d, J=7.49 Hz).

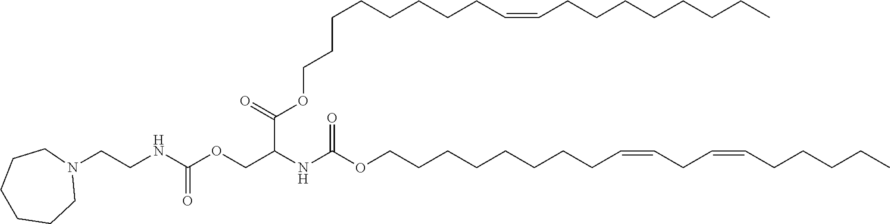 [SMILES: CCCCC/C=C\C/C=C\CCCCCCCCOC(=O)NC(COC(=O)NCCN1CCCCCC1)C(=O)OCCCCCCCC/C=C\CCCCCCCC]

(Z)-octadec-9-en-1-yl O-((2-(azepan-1-yl)ethyl)carbamoyl)-N-((((9Z,12Z)-octadeca-9,12-dien-1-yl)oxy)carbonyl)serinate was synthesized according to representative synthetic scheme 2 and the corresponding methods. MS [ESI]: m/z: [M+H]. calc. 816.7 obs. 817.4; ¹H NMR (400 MHz, CDCl₃): δ 0.88 (6H, m), 1.30 (38H, m), 1.63 (12H, m), 2.04 (8H, m), 2.61 (6H, m), 2.77 (2H, t, J=6.37 Hz), 3.24 (2H, m), 4.06 (2H, t, J=6.97 Hz), 4.15 (2H, t, J=6.79 Hz), 4.35 (1H, dd, J=11.81, 3.01 Hz), 4.44 (1H, dd, J=11.81, 4.19 Hz), 4.55 (1H, m), 5.34 (6H, m), 5.56 (1H, d, J=8.12 Hz).

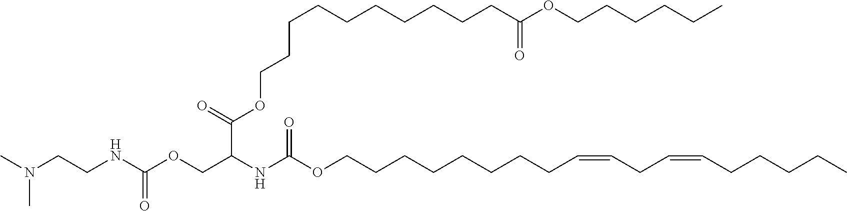

hexyl 11-((O-((2-(dimethylamino)ethyl)carbamoyl)-N-((((9Z,12Z)-octadeca-9,12-dien-1-yl)oxy)carbonyl)seryl)oxy)undecanoate was synthesized according to representative synthetic scheme 2 and the corresponding methods. MS [ESI]: m/z: [M+H]. calc. 780.7 obs. 781.4; ¹H NMR (400 MHz, CDCl₃): δ 0.89 (6H, t, J=6.62 Hz), 1.30 (34H, m), 1.61 (8H, m), 2.04 (4H, q, J=7.09 Hz), 2.22 (6H, s), 2.28 (2H, t, J=7.53 Hz), 2.39 (2H, t, J=5.83 Hz), 2.77 (2H, t, J=6.31 Hz), 3.23 (2H, m), 4.05 (4H, m), 4.15 (2H, t, J=6.72 Hz), 4.34 (1H, dd, J=11.44, 2.86 Hz), 4.42 (1H, dd, J=11.44, 4.07 Hz), 4.54 (1H, m), 5.35 (5H, m), 5.54 (1H, d, J=8.85 Hz).

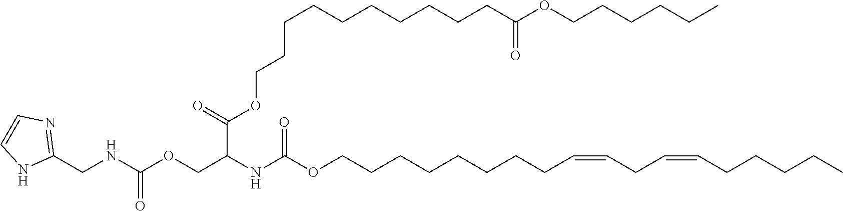

hexyl 11-((O-(((1H-imidazol-2-yl)methyl)carbamoyl)-N-((((9Z,12Z)-octadeca-9,12-dien-1-yl)oxy)carbonyl)seryl)oxy)undecanoate was synthesized according to representative synthetic scheme 2 and the corresponding methods. MS [ESI]: m/z: [M+H]. calc. 789.6 obs. 790.4; ¹H NMR (400 MHz, CDCl₃): δ 0.89 (6H, t, J=7.35 Hz), 1.30 (34H, m), 1.61 (8H, m), 2.05 (4H, q, J=6.95 Hz), 2.29 (2H, t, J=7.45 Hz), 2.77 (2H, t, J=6.51 Hz), 4.06 (6H, m), 4.43 (4H, m), 4.65 (1H, m), 5.35 (4H, m), 5.55 (1H, m), 5.75 (1H, m), 7.01 (2H, m).

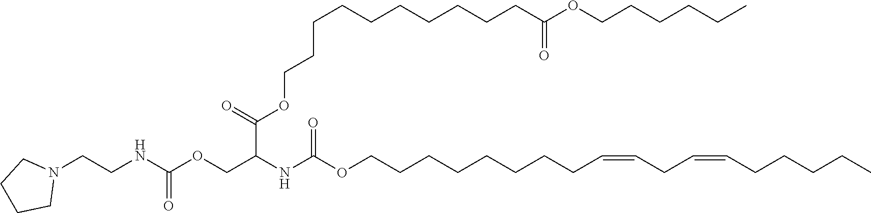 [SMILES: CCCCC/C=C\C/C=C\CCCCCCCCOC(=O)NC(COC(=O)NCCN1CCCC1)C(=O)OCCCCCCCCCCC(=O)OCCCCCC]

hexyl 11-((N-((((9Z,12Z)-octadeca-9,12-dien-1-yl)oxy)carbonyl)-O-((2-(pyrrolidin-1-yl)ethyl)carbamoyl)seryl)oxy)undecanoate was synthesized according to representative synthetic scheme 2 and the corresponding methods. MS [ESI]: m/z: [M+H]. calc. 806.6 obs. 806.9. ¹H NMR (400 MHz, CDCl₃): δ 0.89 (6H, t, J=7.05 Hz), 1.30 (34H, m), 1.61 (8H, m), 1.83 (4H, m), 2.05 (4H, q, J=7.17 Hz), 2.28 (2H, t, J=7.94 Hz), 2.63 (6H, m), 2.77 (2H, t, J=6.44 Hz), 3.32 (2H, m), 4.05 (4H, m), 4.15 (2H, t, J=6.81 Hz), 4.34 (1H, m), 4.44 (1H, m), 4.54 (1H, m), 5.35 (4H, m), 5.50 (1H, m), 5.60 (1H, d, J=8.04 Hz).

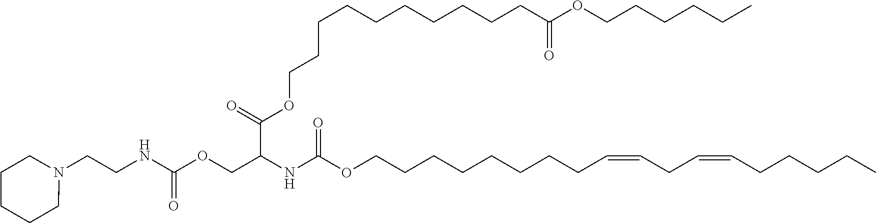 [SMILES: CCCCC/C=C\C/C=C\CCCCCCCCOC(=O)NC(COC(=O)NCCN1CCCCC1)C(=O)OCCCCCCCCCCC(=O)OCCCCCC]

hexyl 11-((N-((((9Z,12Z)-octadeca-9,12-dien-1-yl)oxy)carbonyl)-O-((2-(piperidin-1-yl)ethyl)carbamoyl)seryl)oxy)undecanoate was synthesized according to representative synthetic scheme 2 and the corresponding methods. ¹H NMR (400 MHz, CDCl₃): δ 0.89 (6H, t, J=6.92 Hz), 1.30 (34H, m), 1.46 (2H, m), 1.61 (12H, m), 2.04 (4H, q, J=6.41 Hz), 2.28 (2H, t, J=7.43 Hz), 2.43 (6H, m), 2.77 (2H, t, J=6.43 Hz), 3.27 (2H, m), 4.05 (4H, m), 4.15 (2H, t, J=6.85 Hz), 4.34 (1H, dd, J=11.50, 3.45 Hz), 4.44 (1H, dd, J=11.50, 3.76 Hz), 4.55 (1H, m), 5.35 (5H, m), 5.56 (1H, d, J=8.45 Hz). MS [ESI]: m/z: [M+H] calc. 820.6 obs. 820.6.

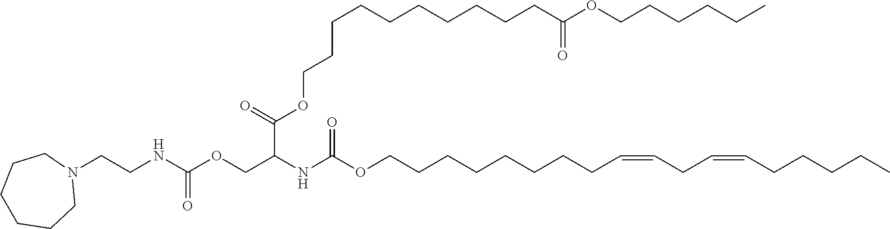 [SMILES: CCCCC/C=C\C/C=C\CCCCCCCCOC(=O)NC(COC(=O)NCCN1CCCCCC1)C(=O)OCCCCCCCCCCC(=O)OCCCCCC]

hexyl 11-((O-((2-(azepan-1-yl)ethyl)carbamoyl)-N-((((9Z,12Z)-octadeca-9,12-dien-1-yl)oxy)carbonyl)seryl)oxy)undecanoate was synthesized according to representative synthetic scheme 2 and the corresponding methods. ¹H NMR (400 MHz, CDCl₃): δ 0.89 (6H, t, J=6.53 Hz), 1.30 (34H, m), 1.61 (16H, m), 2.05 (4H, q, J=7.43 Hz), 2.28 (2H, t, J=7.94 Hz), 2.77 (8H, m), 3.27 (2H, m), 4.05 (4H, m), 4.15 (2H, t, J=6.70 Hz), 4.34 (1H, dd, J=11.44, 3.43 Hz), 4.44 (1H, dd, J=11.44, 3.89 Hz), 4.55 (1H, m), 5.35 (4H, m), 5.58 (1H, m). MS [ESI]: m/z: [M+H] calc. 834.6 obs. 820.5.

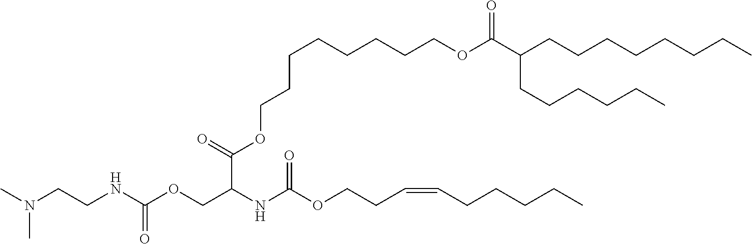 [SMILES: CCCCC/C=C\CCOC(=O)NC(COC(=O)NCCN(C)C)C(=O)OCCCCCCCCOC(=O)C(CCCCCC)CCCCCCCC]

(Z)-2-methyl-9-(((non-3-en-1-yloxy)carbonyl)amino)-6,10-dioxo-7,11-dioxa-2,5-diazanonadecan-19-yl 2-hexyldecanoate 754.5 was synthesized according to representative synthetic scheme 2 and the corresponding methods. ¹H NMR (400 MHz, CDCl₃): δ 0.88 (9H, m), 1.25 (36H, m), 1.51-1.70 (6H, m), 2.03 (2H, q, J=7.56 Hz), 2.22 (11H, m), 3.22 (2H, m), 4.06 (4H, m), 4.15 (2H, t, J=7.08 Hz), 4.29-4.48 (2H, m), 4.54 (1H, m), 5.29 (2H, m), 5.45-5.60 (2H, m). MS [ESI]: m/z: [M+H] calc. 754.5 obs. 754.3.

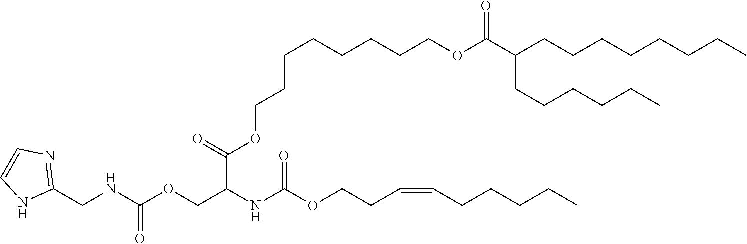 [SMILES: CCCCC/C=C\CCOC(=O)NC(COC(=O)NCc1ncc[nH]1)C(=O)OCCCCCCCCOC(=O)C(CCCCCC)CCCCCCCC]

((Z)-8-((O-(((1H-imidazol-2-yl)methyl)carbamoyl)-N-((non-3-en-1-yloxy)carbonyl)seryl)oxy)octyl 2-hexyldecanoate was synthesized according to representative synthetic scheme 2 and the corresponding methods. ¹H NMR (400 MHz, CDCl₃): δ 0.88 (9H, m), 1.25 (36H, m), 1.51-1.70 (6H, m), 2.03 (2H, q, J=7.56 Hz), 2.22 (11H, m), 3.22 (2H, m), 4.06 (4H, m), 4.15 (2H, t, J=7.08 Hz), 4.29-4.48 (2H, m), 4.54 (1H, m), 5.29 (2H, m), 5.45-5.60 (2H, m). MS [ESI]: m/z: [M+H] calc. 765.3 obs. 763.4.

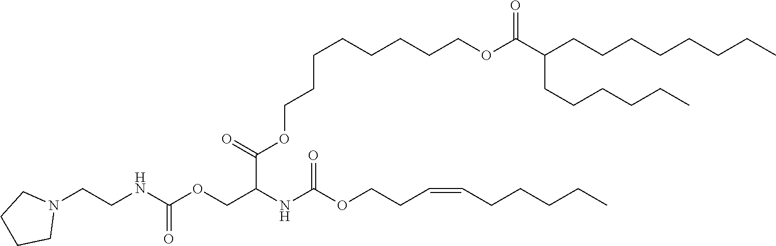 [SMILES: CCCCC/C=C\CCOC(=O)NC(COC(=O)NCCN1CCCC1)C(=O)OCCCCCCCCOC(=O)C(CCCCCC)CCCCCCCC]

((Z)-8-((N-((non-3-en-1-yloxy)carbonyl)-O-((2-(pyrrolidin-1-yl)ethyl)carbamoyl)seryl)oxy)octyl 2-hexyldecanoate was synthesized according to representative synthetic scheme 2 and the corresponding methods. ¹H NMR (400 MHz, CDCl₃): δ 0.88 (9H, m), 1.26 (34H, m), 1.52-1.72 (8H, m), 1.78 (4H, m), 2.04 (2H, q, J=7.09 Hz), 2.37 (4H, m), 2.46-2.53 (3H, m), 2.57 (2H, t, J=6.00 Hz), 3.22-3.31 (2H, m), 4.06 (4H, m), 4.16 (2H, t, J=6.67 Hz), 4.31-4.47 (2H, m), 4.51-4.59 (1H, m), 5.25-5.42 (2H, m), 5.45-5.61 (2H, m). MS [ESI]: m/z: [M+H] calc. 780.6 obs. 780.2.

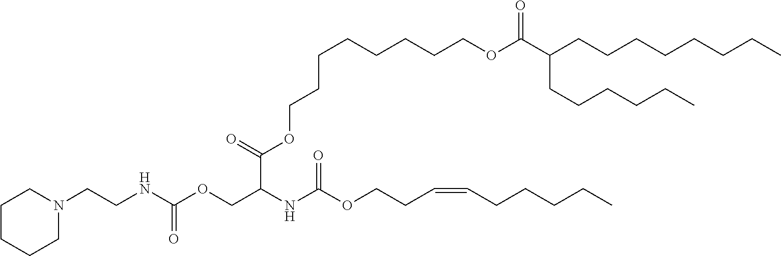 [SMILES: CCCCC/C=C\CCOC(=O)NC(COC(=O)NCCN1CCCCC1)C(=O)OCCCCCCCCOC(=O)C(CCCCCC)CCCCCCCC]

(Z)-8-((O-((2-(azepan-1-yl)ethyl)carbamoyl)-N-((non-3-en-1-yloxy)carbonyl)seryl)oxy)octyl 2-hexyldecanoate was synthesized according to representative synthetic scheme 2 and the corresponding methods. ¹H NMR (400 MHz, CDCl₃): δ 0.87 (9H, m), 1.25 (34H, m), 1.49-1.71 (14H, m), 2.03 (2H, q, J=6.83 Hz), 2.37 (9H, m), 3.24 (2H, m), 4.06 (4H, q, J=6.58 Hz), 4.15 (2H, t, J=6.75 Hz), 4.27-4.49 (2H, m), 4.55 (1H, m), 5.18-5.41 (2H, m), 5.44-5.60 (2H, m). MS [ESI]: m/z: [M+H] calc. 794.6 obs. 780.2.

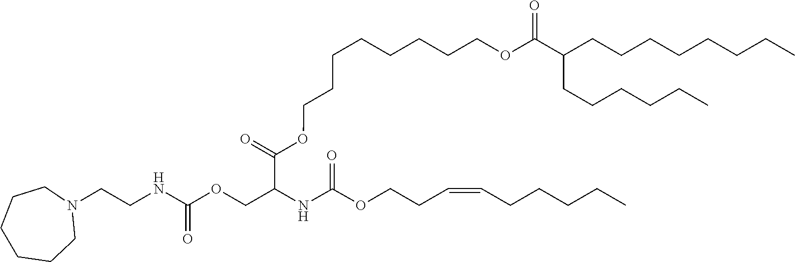 [SMILES: CCCCC/C=C\CCOC(=O)NC(COC(=O)NCCN1CCCCCC1)C(=O)OCCCCCCCCOC(=O)C(CCCCCC)CCCCCCCC]

(Z)-8-((O-((2-(azepan-1-yl)ethyl)carbamoyl)-N-((non-3-en-1-yloxy)carbonyl)seryl)oxy)octyl 2-hexyldecanoate was synthesized according to representative synthetic scheme 2 and the corresponding methods. ¹H NMR (400 MHz, CDCl₃): δ 0.88 (9H, m), 1.25 (34H, m), 1.60 (16H, m), 2.03 (2H, q, J=8.03 Hz), 2.24-2.34 (2H, m), 2.37 (2H, q, J=7.86 Hz), 2.62 (5H, m), 3.20 (2H, m), 4.06 (4H, q, J=6.75 Hz), 4.16 (2H, t, J=7.03 Hz), 4.40 (2H, dd, J=42.91, 12.16 Hz), 4.49-4.62 (1H, m), 5.19-5.41 (2H, m), 5.44-5.61 (2H, m). MS [ESI]: m/z: [M+H] calc. 808.6 obs. 808.5.

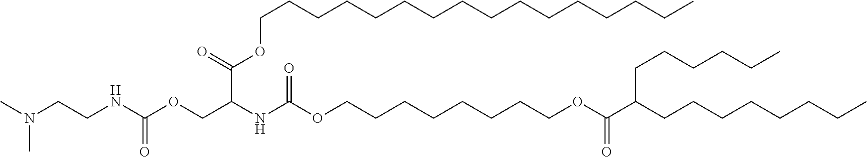 [SMILES: CCCCCCCCCCCCCCCCOC(=O)C(COC(=O)NCCN(C)C)NC(=O)OCCCCCCCCOC(=O)C(CCCCCC)CCCCCCCC]

9-((hexadecyloxy)carbonyl)-2-methyl-6,11-dioxo-7,12-dioxa-2,5,10-triazaicosan-20-yl 2-hexyldecanoate was synthesized according to representative synthetic scheme 2 and the corresponding methods. ¹H NMR (400 MHz, CDCl₃): δ 0.87 (9H, m), 1.26 (52H, m), 1.38-1.48 (2H, m), 1.62 (10H, m), 2.31 (2H, m), 2.61 (5H, m), 2.77-2.97 (2H, m), 3.40-3.53 (2H, m), 4.06 (4H, t, J=7.05 Hz), 4.11-4.19 (2H, m), 4.31-4.50 (2H, m), 4.51-4.60 (1H, m), 5.73-5.82 (1H, m). MS [ESI]: m/z: [M+H] calc. 854.7 obs. 857.7.

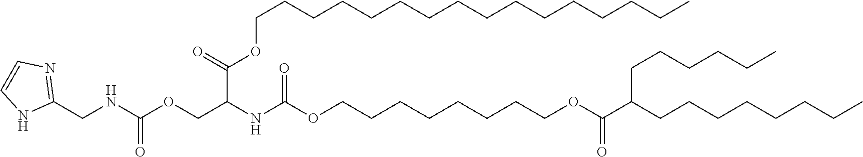 [SMILES: CCCCCCCCCCCCCCCCOC(=O)C(COC(=O)NCc1ncc[nH]1)NC(=O)OCCCCCCCCOC(=O)C(CCCCCC)CCCCCCCC]

6-((hexadecyloxy)carbonyl)-1-(1H-imidazol-2-yl)-3,8-dioxo-4,9-dioxa-2,7-diazaheptadecan-17-yl 2-hexyldecanoate was synthesized according to representative synthetic scheme 2 and the corresponding methods. ¹H NMR (400 MHz, CDCl₃): δ 0.87 (9H, m), 1.25 (56H, m), 1.61 (8H, m), 2.30 (1H, m), 4.06 (4H, t, J=7.17 Hz), 4.11-4.28 (3H, m), 4.42-4.60 (2H, m), 4.60-4.78 (3H, m), 5.62-5.76 (1H, m), 6.32-6.44 (1H, m), 7.11 (2H, m). MS [ESI]: m/z: [M+H] calc. 863.6 obs. 863.7.

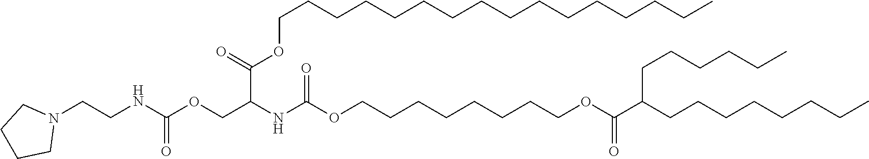 [SMILES: CCCCCCCCCCCCCCCCOC(=O)C(COC(=O)NCCN1CCCC1)NC(=O)OCCCCCCCCOC(=O)C(CCCCCC)CCCCCCCC]

7-((hexadecyloxy)carbonyl)-4,9-dioxo-1-(pyrrolidin-1-yl)-5,10-dioxa-3,8-diazaoctadecan-18-yl 2-hexyldecanoate was synthesized according to representative synthetic scheme 2 and the corresponding methods. ¹H NMR (400 MHz, CDCl₃): δ 0.87 (9H, m), 1.26 (52H, m), 1.61 (10H, m), 1.96 (6H, m), 2.30 (1H, m), 2.67-3.02 (6H, m), 3.44 (2H, m), 4.06 (4H, t, J=6.92 Hz), 4.15 (2H, t, J=6.97 Hz), 4.30-4.50 (2H, m), 4.50-4.60 (1H, m), 5.62-5.79 (1H, m). MS [ESI]: m/z: [M+H] calc. 880.7 obs. 880.8.

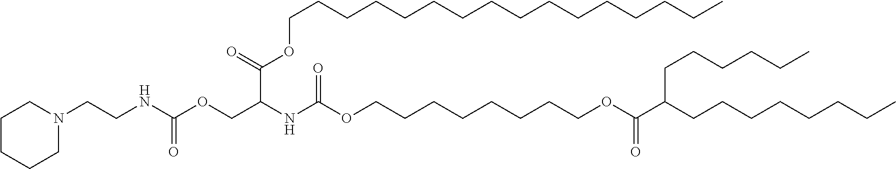 [SMILES: CCCCCCCCCCCCCCCCOC(=O)C(COC(=O)NCCN1CCCCC1)NC(=O)OCCCCCCCCOC(=O)C(CCCCCC)CCCCCCCC]

7-((hexadecyloxy)carbonyl)-4,9-dioxo-1-(piperidin-1-yl)-5,10-dioxa-3,8-diazaoctadecan-18-yl 2-hexyldecanoate was synthesized according to representative synthetic scheme 2 and the corresponding methods. ¹H NMR (400 MHz, CDCl₃): δ 0.87 (9H, m), 1.26 (54H, m), 1.61 (16H, m), 2.30 (1H, m), 2.36-3.15 (6H, m), 3.17-3.71 (2H, m), 4.06 (4H, t, J=7.04 Hz), 4.15 (2H, t, J=6.97 Hz), 4.29-4.50 (2H, m), 4.55 (1H, m), 5.58-5.81 (1H, m). MS [ESI]: m/z: [M+H] calc. 894.7 obs. 894.8.

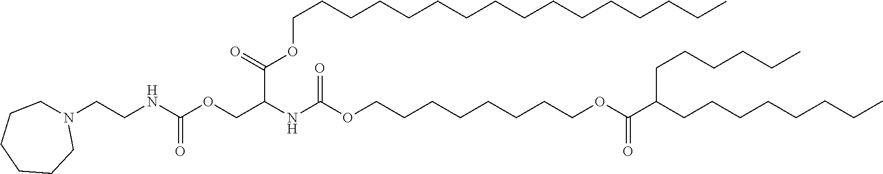 [SMILES: CCCCCCCCCCCCCCCCOC(=O)C(COC(=O)NCCN1CCCCCC1)NC(=O)OCCCCCCCCOC(=O)C(CCCCCC)CCCCCCCC]

1-(azepan-1-yl)-7-((hexadecyloxy)carbonyl)-4,9-dioxo-5,10-dioxa-3,8-diazaoctadecan-18-yl 2-hexyldecanoate was synthesized according to representative synthetic scheme 2 and the corresponding methods. ¹H NMR (400 MHz, CDCl₃): δ 0.87 (9H, m), 1.26 (54H, m), 1.62 (18H, m), 2.30 (1H, m), 2.88 (6H, m), 3.28-3.62 (2H, m), 4.06 (4H, m), 4.15 (2H, t, J=6.79 Hz), 4.27-4.49 (2H, m), 4.55 (1H, m), 5.58-5.72 (1H, m). MS [ESI]: m/z: [M+H] calc. 908.7 obs. 908.8.

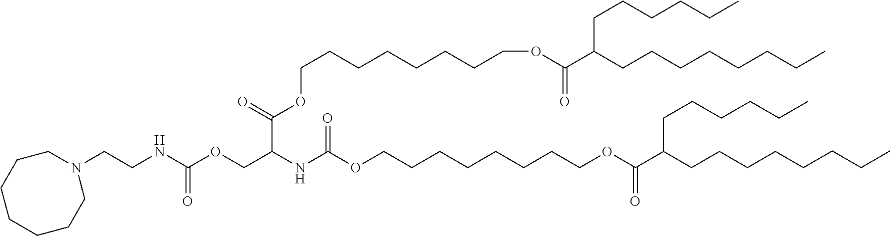 [SMILES: CCCCCCCCC(CCCCCC)C(=O)OCCCCCCCCOC(=O)NC(COC(=O)NCCN1CCCCCCC1)C(=O)OCCCCCCCCOC(=O)C(CCCCCC)CCCCCCCC]

11-((((2-(azocan-1-yl)ethyl)carbamoyl)oxy)methyl)-25-hexyl-10,13,24-trioxo-9,14,23-trioxa-12-azatritriacontyl 2-hexyldecanoate was synthesized according to representative synthetic scheme 2 and the corresponding methods. ¹H NMR (400 MHz, CDCl₃): δ 0.87 (12H, t, J=7.01 Hz), 1.25 (62H, m), 1.61 (22H, m), 2.31 (2H, m), 2.62 (2H, m), 3.10 (2H, m), 3.58 (2H, m), 4.06 (6H, t, J=7.02 Hz), 4.15 (2H, m), 4.35 (1H, dd, J=11.67, 3.25 Hz), 4.45 (1H, dd, J=11.67, 3.93 Hz), 4.55 (1H, m). MS [ESI]: m/z: [M+H] calc. 1064.8 obs. 1064.7.

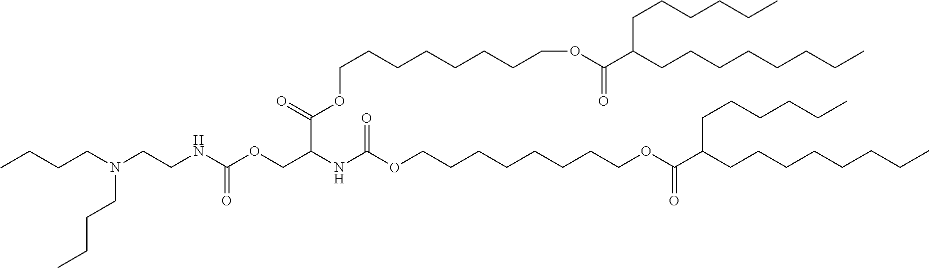 [SMILES: CCCCCCCCC(CCCCCC)C(=O)OCCCCCCCCOC(=O)NC(COC(=O)NCCN(CCCC)CCCC)C(=O)OCCCCCCCCOC(=O)C(CCCCCC)CCCCCCCC]

5-butyl-12-(((8-((2-hexyldecanoyl)oxy)octyl)oxy)carbonyl)-9,14-dioxo-10,15-dioxa-5,8,13-triazatricosan-23-yl 2-hexyldecanoate was synthesized according to representative synthetic scheme 2 and the corresponding methods. ¹H NMR (400 MHz, CDCl₃): δ 0.87 (18H, m), 1.25 (64H, m), 1.60 (16H, m), 2.31 (4H, m), 3.05 (4H, m), 3.58 (2H, m), 4.06 (6H, t, J=6.61 Hz), 4.15 (2H, m), 4.34 (1H, m), 4.45 (1H, m), 4.55 (1H, m), 5.63 (2H, m). MS [ESI]: m/z: [M+H] calc. 1080.9 obs. 1080.9.

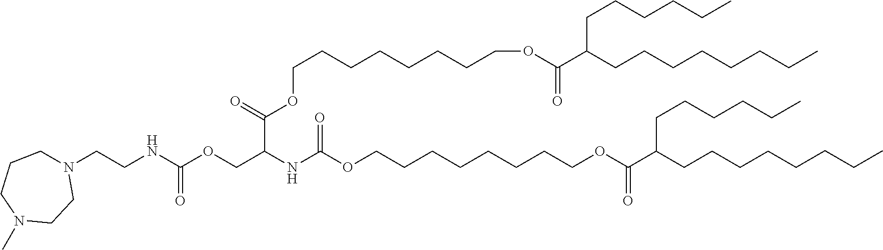 [SMILES: CCCCCCCCC(CCCCCC)C(=O)OCCCCCCCCOC(=O)NC(COC(=O)NCCN1CCCN(C)CC1)C(=O)OCCCCCCCCOC(=O)C(CCCCCC)CCCCCCCC]

(Z)-8-((O-((2-(azepan-1-yl)ethyl)carbamoyl)-N-((non-3-en-1-yloxy)carbonyl)seryl)oxy)octyl 2-hexyldecanoate was synthesized according to representative synthetic scheme 2 and the corresponding methods. ¹H NMR (400 MHz, CDCl₃): δ 0.87 (12H, t, J=6.89 Hz), 1.25 (60H, m), 1.50-1.70 (14H, m), 2.30 (2H, m), 2.56-2.82 (6H, m), 2.82-3.10 (5H, m), 3.23 (2H, m), 4.06 (6H, t, J=6.61 Hz), 4.15 (2H, m), 4.28-4.61 (3H, m), 5.45-5.61 (1H, m), 5.62-5.75 (1H, m). MS [ESI]: m/z: [M+H] calc. 1065.8 obs. 1065.8.

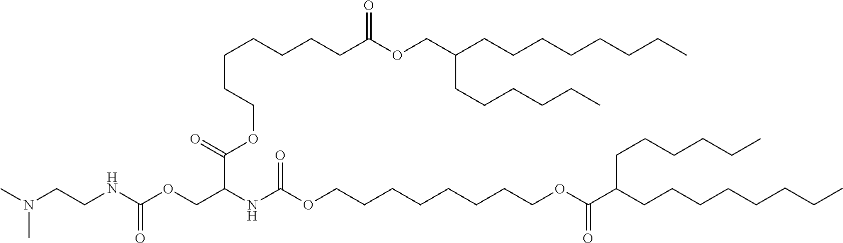 [SMILES: CCCCCCCCC(CCCCCC)COC(=O)CCCCCCCOC(=O)C(COC(=O)NCCN(C)C)NC(=O)OCCCCCCCCOC(=O)C(CCCCCC)CCCCCCCC]

(9-(((8-((2-hexyldecyl)oxy)-8-oxooctyl)oxy)carbonyl)-2-methyl-6,11-dioxo-7,12-dioxa-2,5,10-triazaicosan-20-yl 2-hexyldecanoate was synthesized according to representative synthetic scheme 2 and the corresponding methods. ¹H NMR (400 MHz, CDCl₃): δ 0.87 (12H, m), 1.26 (56H, m), 1.60 (15H, m), 2.21 (6H, s), 2.29 (3H, t, J=7.57 Hz), 2.38 (2H, t, J=5.78 Hz), 3.23 (2H, m), 3.96 (2H, d, J=5.64 Hz), 4.06 (4H, t, J=6.68 Hz), 4.15 (2H, t, J=6.76 Hz), 4.31-4.47 (2H, m), 4.49-4.60 (1H, m), 5.18-5.27 (1H, m), 5.47-5.56 (1H, m). MS [ESI]: m/z: [M+H] calc. 996.8 obs. 996.7.

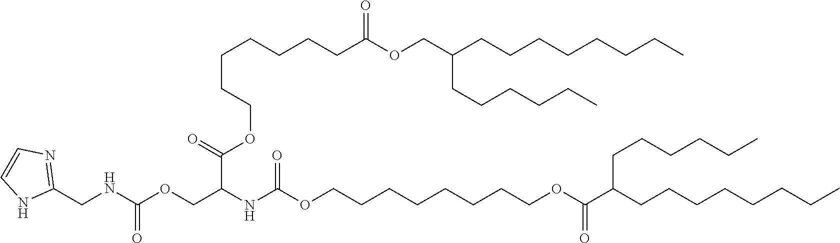 [SMILES: CCCCCCCCC(CCCCCC)COC(=O)CCCCCCCOC(=O)C(COC(=O)NCc1ncc[nH]1)NC(=O)OCCCCCCCCOC(=O)C(CCCCCC)CCCCCCCC]

12-(((((1H-imidazol-2-yl)methyl)carbamoyl)oxy)methyl)-25-hexyl-10,13,22-trioxo-9,14,23-trioxa-11-azatritriacontyl 2-hexyldecanoate was synthesized according to representative synthetic scheme 2 and the corresponding methods. ¹H NMR (400 MHz, CDCl₃): δ 0.88 (12H, m), 1.26 (58H, m), 1.61 (13H, m), 2.29 (3H, t, J=7.94 Hz), 3.97 (2H, d, J=5.72 Hz), 4.06 (4H, t, J=6.55 Hz), 4.15 (2H, t, J=6.40 Hz), 4.30-4.52 (4H, m), 4.63 (1H, m), 5.51 (2H, m), 6.97 (2H, m). MS [ESI]: m/z: [M+H] calc. 1005.8 obs. 1005.8.

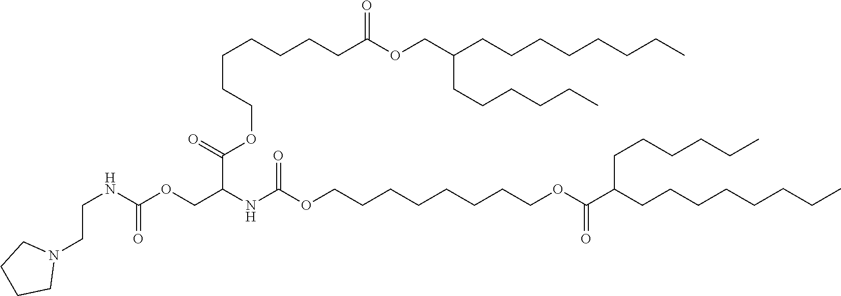 [SMILES: CCCCCCCCC(CCCCCC)COC(=O)CCCCCCCOC(=O)C(COC(=O)NCCN1CCCC1)NC(=O)OCCCCCCCCOC(=O)C(CCCCCC)CCCCCCCC]

(25-hexyl-10,13,22-trioxo-12-((((2-(pyrrolidin-1-yl)ethyl)carbamoyl)oxy)methyl)-9,14,23-trioxa-11-azatritriacontyl 2-hexyldecanoate was synthesized according to representative synthetic scheme 2 and the corresponding methods. ¹H NMR (400 MHz, CDCl₃): δ 0.85 (12H, m), 1.26 (58H, m), 1.61 (17H, m), 2.28 (3H, t, J=7.94 Hz), 2.49 (4H, m), 2.57 (2H, t, J=5.85 Hz), 3.26 (2H, m), 3.96 (2H, d, J=5.81 Hz), 4.06 (4H, t, J=6.87 Hz), 4.15 (2H, t, J=6.94 Hz), 4.28-4.48 (2H, m), 4.53 (1H, m), 5.30 (1H, m), 5.54 (1H, d, J=8.11 Hz). MS [ESI]: m/z: [M+H] calc. 1022.8 obs. 1022.9.

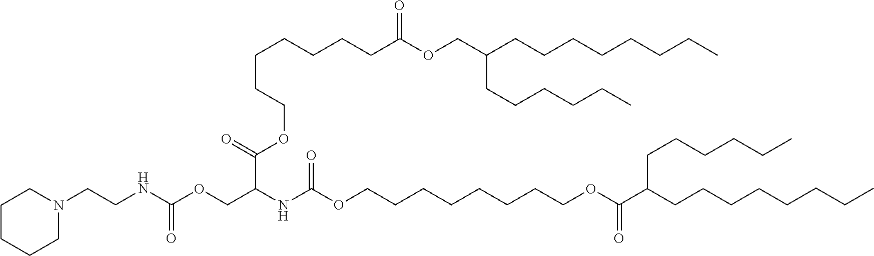 [SMILES: CCCCCCCCC(CCCCCC)COC(=O)CCCCCCCOC(=O)C(COC(=O)NCCN1CCCCC1)NC(=O)OCCCCCCCCOC(=O)C(CCCCCC)CCCCCCCC]

25-hexyl-10,13,22-trioxo-12-((((2-(piperidin-1-yl)ethyl)carbamoyl)oxy)methyl)-9,14,23-trioxa-11-azatritriacontyl 2-hexyldecanoate was synthesized according to representative synthetic scheme 2 and the corresponding methods. ¹H NMR (400 MHz, CDCl₃): δ 0.88 (12H, m), 1.26 (58H, m), 1.61 (19H, m), 2.24-2.45 (9H, m), 3.24 (2H, m), 3.96 (2H, d, J=5.72 Hz), 4.06 (4H, t, J=7.03 Hz), 4.15 (2H, t, J=6.77 Hz), 4.29-4.48 (211, m), 4.54 (1H, m), 5.26 (1H, m), 5.55 (1H, d, J=8.63 Hz). MS [ESI]: m/z: [M+H] calc. 1035.8 obs. 1036.6.

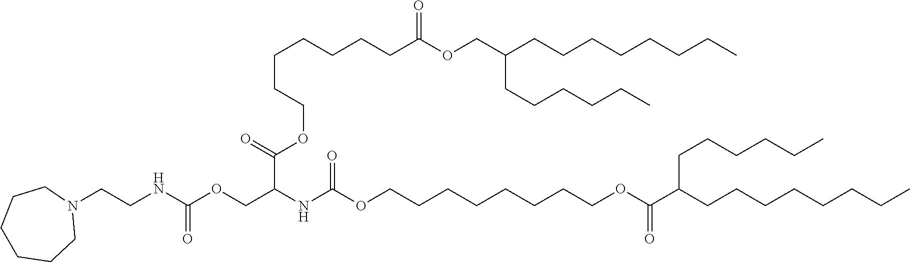 [SMILES: CCCCCCCCC(CCCCCC)COC(=O)CCCCCCCOC(=O)C(COC(=O)NCCN1CCCCCC1)NC(=O)OCCCCCCCCOC(=O)C(CCCCCC)CCCCCCCC]

(Z)-8-((O-((2-(azepan-1-yl)ethyl)carbamoyl)-N-((non-3-en-1-yloxy)carbonyl)seryl)oxy)octyl 2-hexyldecanoate was synthesized according to representative synthetic scheme 2 and the corresponding methods. ¹H NMR (400 MHz, CDCl₃): δ 0.88 (12H, m), 1.26 (56H, m), 1.60 (23H, m), 2.29 (3H, t, J=7.60 Hz), 2.62 (6H, m), 3.20 (2H, m), 3.96 (2H, d, J=5.81 Hz), 4.06 (4H, s), 4.15 (2H, t, J=6.72 Hz), 4.29-4.48 (211, m), 4.50-4.60 (1H, m), 5.22-5.34 (1H, m), 5.54 (1H, d, J=8.11 Hz). MS [ESI]: m/z: [M+H] calc. 1050.8 obs. 1050.7.

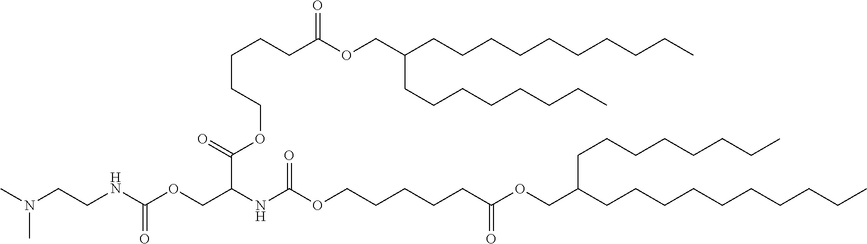 [SMILES: CCCCCCCCCCC(CCCCCCCC)COC(=O)CCCCCOC(=O)NC(COC(=O)NCCN(C)C)C(=O)OCCCCCC(=O)OCC(CCCCCCCC)CCCCCCCCCC]

2-octyldodecyl 2-methyl-9-(((6-((2-octyldodecyl)oxy)-6-oxohexyl)oxy)carbonyl)-6,11-dioxo-7,12-dioxa-2,5,10-triazaoctadecan-18-oate was synthesized according to representative synthetic scheme 2 and the corresponding methods. ¹H NMR (400 MHz, CDCl₃): δ 0.88 (12H, t, J=6.70 Hz), 1.26 (64H, m), 1.63 (14H, m), 2.22 (6H, s), 2.31 (4H, t, J=7.49 Hz), 2.39 (2H, t, J=5.74 Hz), 3.20-3.30 (2H, m), 3.96 (4H, d, J=5.81 Hz), 4.07 (2H, t, J=6.89 Hz), 4.16 (2H, td, J=6.58, 2.14 Hz), 4.38 (2H, dd, J=34.97, 11.44 Hz), 4.53 (1H, m), 5.30 (1H, m), 5.55 (1H, d, J=8.11 Hz). MS [ESI]: m/z: [M+H] calc. 1052.9 obs. 1052.7.

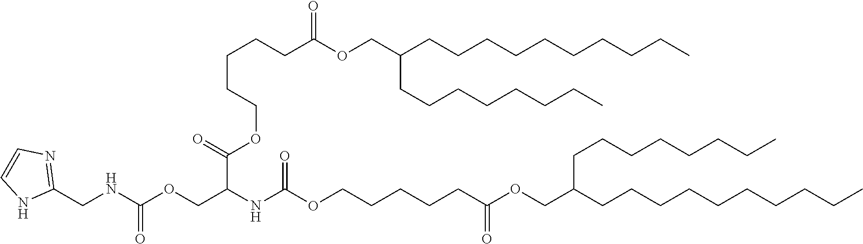 [SMILES: CCCCCCCCCCC(CCCCCCCC)COC(=O)CCCCCOC(=O)NC(COC(=O)NCc1ncc[nH]1)C(=O)OCCCCCC(=O)OCC(CCCCCCCC)CCCCCCCCCC]

2-octyldodecyl 1-(1H-imidazol-2-yl)-6-(((6-((2-octyldodecyl)oxy)-6-oxohexyl)oxy)carbonyl)-3,8-dioxo-4,9-dioxa-2,7-diazapentadecan-15-oate was synthesized according to representative synthetic scheme 2 and the corresponding methods. ¹H NMR (400 MHz, CDCl₃): δ 0.88 (12H, t, J=7.42 Hz), 1.26 (64H, m), 1.64 (14H, m), 2.30 (4H, t, J=7.34 Hz), 3.96 (4H, d, J=5.64 Hz), 4.01-4.23 (4H, m), 4.40 (4H, m), 4.63 (1H, m), 5.55 (1H, d, J=8.03 Hz), 5.79 (1H, m), 7.11 (2H, s), 7.69 (1H, s). MS [ESI]: m/z: [M+H] calc. 1061.8 obs. 1061.7.

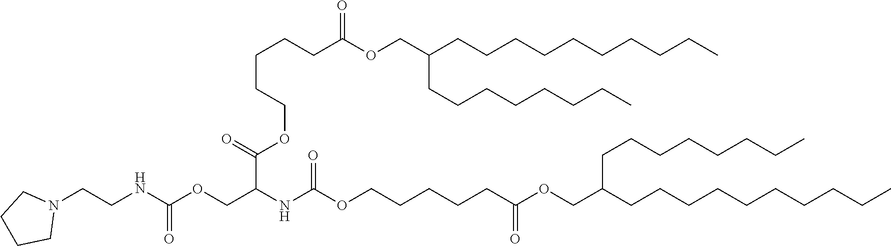 [SMILES: CCCCCCCCCCC(CCCCCCCC)COC(=O)CCCCCOC(=O)NC(COC(=O)NCCN1CCCC1)C(=O)OCCCCCC(=O)OCC(CCCCCCCC)CCCCCCCCCC]

2-octyldodecyl 7-(((6-((2-octyldodecyl)oxy)-6-oxohexyl)oxy)carbonyl)-4,9-dioxo-1-(pyrrolidin-1-yl)-5,10-dioxa-3,8-diazahexadecan-16-oate was synthesized according to representative synthetic scheme 2 and the corresponding methods. ¹H NMR (400 MHz, CDCl₃): δ 0.88 (12H, t, J=6.83 Hz), 1.26 (66H, m), 1.56-1.73 (12H, m), 1.77 (4H, m), 2.31 (4H, t, J=7.86 Hz), 2.50 (4H, m), 2.56 (2H, t, J=6.66 Hz), 3.27 (2H, m), 3.96 (4H, d, J=5.83 Hz), 4.07 (2H, t, J=6.95 Hz), 4.16 (2H, t, J=6.57 Hz), 4.31-4.47 (2H, m), 4.49-4.60 (1H, m), 5.33 (1H, m), 5.56 (1H, d, J=8.88 Hz). MS [ESI]: m/z: [M+H] calc. 1078.9 obs. 1078.7.

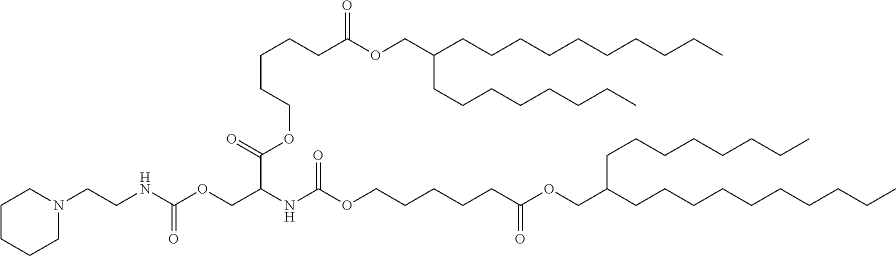 [SMILES: CCCCCCCCCCC(CCCCCCCC)COC(=O)CCCCCOC(=O)NC(COC(=O)NCCN1CCCCC1)C(=O)OCCCCCC(=O)OCC(CCCCCCCC)CCCCCCCCCC]

2-octyldodecyl 7-(((6-((2-octyldodecyl)oxy)-6-oxohexyl)oxy)carbonyl)-4,9-dioxo-1-(piperidin-1-yl)-5,10-dioxa-3,8-diazahexadecan-16-oate was synthesized according to representative synthetic scheme 2 and the corresponding methods. ¹H NMR (400 MHz, CDCl₃): δ 0.88 (12H, m), 1.26 (60H, m), 1.35-1.49 (6H, m), 1.51-1.73 (18H, m), 2.31 (4H, t, J=7.65 Hz), 2.34-2.38 (4H, m), 2.41 (2H, t, J=6.10 Hz), 3.19-3.30 (2H, m), 3.96 (4H, d, J=5.90 Hz), 4.07 (2H, t, J=6.78 Hz), 4.17 (2H, t, J=6.79 Hz), 4.39 (2H, dd, J=41.29, 12.11 Hz), 4.49-4.59 (1H, m), 5.31 (1H, m), 5.56 (1H, d, J=8.20 Hz). MS [ESI]: m/z: [M+H] calc. 1092.8 obs. 1092.8.

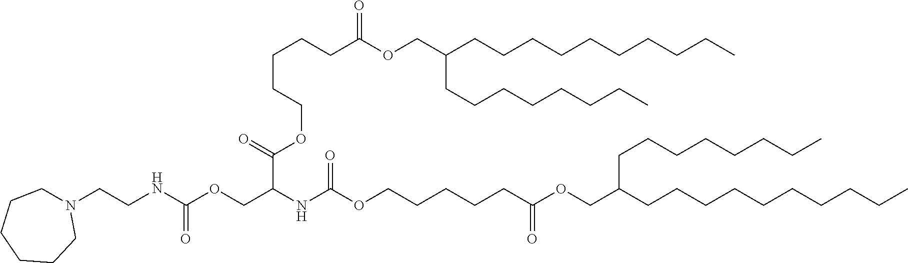 [SMILES: CCCCCCCCCCC(CCCCCCCC)COC(=O)CCCCCOC(=O)NC(COC(=O)NCCN1CCCCCC1)C(=O)OCCCCCC(=O)OCC(CCCCCCCC)CCCCCCCCCC]

(Z)-8-((O-((2-(azepan-1-yl)ethyl)carbamoyl)-N-((non-3-en-1-yloxy)carbonyl)seryl)oxy)octyl 2-hexyldecanoate was synthesized according to representative synthetic scheme 2 and the corresponding methods. ¹H NMR (400 MHz, CDCl₃): δ 0.88 (12H, t, J=6.58 Hz), 1.26 (60H, m), 1.34-1.45 (6H, m), 1.53-1.73 (20H, m), 2.31 (4H, t, J=7.94 Hz), 2.53-2.66 (6H, m), 3.20 (2H, m), 3.96 (4H, d, J=5.81 Hz), 4.07 (2H, t, J=6.70 Hz), 4.16 (2H, t, J=6.62 Hz), 4.40 (2H, dd, J=41.21, 10.77 Hz), 4.49-4.59 (1H, m), 5.33 (1H, m), 5.57 (1H, d, J=8.28 Hz). MS [ESI]: m/z: [M+H] calc. 1106.9 obs. 1106.9.

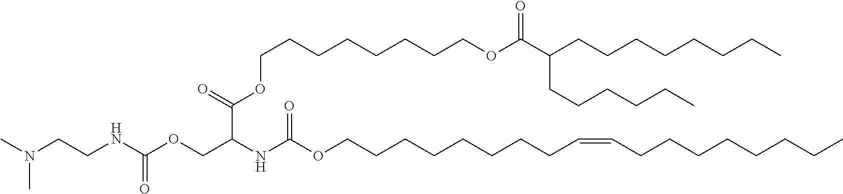 [SMILES: CCCCCCCC/C=C\CCCCCCCCOC(=O)NC(COC(=O)NCCN(C)C)C(=O)OCCCCCCCCOC(=O)C(CCCCCC)CCCCCCCC]

(Z)-2-methyl-9-(((octadec-9-en-1-yloxy)carbonyl)amino)-6,10-dioxo-7,11-dioxa-2,5-diazanonadecan-19-yl 2-hexyldecanoate was synthesized according to representative synthetic scheme 2 and the corresponding methods. ¹H NMR (400 MHz, CDCl₃): δ 0.87 (9H, m), 1.25 (52H, m), 1.62 (8H, m), 2.01 (4H, m), 2.32 (7H, m), 2.51 (2H, m), 3.29 (2H, m), 4.06 (4H, m), 4.15 (2H, t, J=6.75 Hz), 4.31-4.49 (2H, m), 4.54 (1H, m), 5.34 (2H, m), 5.40-5.53 (1H, m), 5.58 (1H, d, J=7.86 Hz). MS [ESI]: m/z: [M+H] calc. 880.7 obs. 880.7.

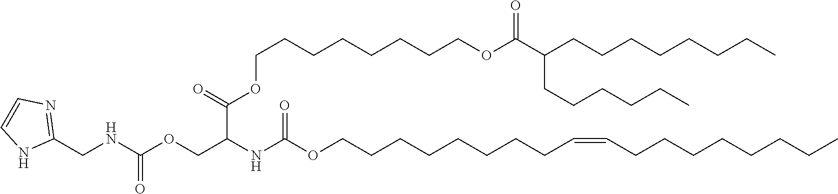 [SMILES: CCCCCCCC/C=C\CCCCCCCCOC(=O)NC(COC(=O)NCc1ncc[nH]1)C(=O)OCCCCCCCCOC(=O)C(CCCCCC)CCCCCCCC]

((Z)-8-((O-(((1H-imidazol-2-yl)methyl)carbamoyl)-N-((octadec-9-en-1-yloxy)carbonyl)seryl)oxy)octyl 2-hexyldecanoate was synthesized according to representative synthetic scheme 2 and the corresponding methods. ¹H NMR (400 MHz, CDCl₃): δ 0.87 (9H, m), 1.25 (50H, m), 1.61 (10H, m), 2.02 (3H, m), 2.31 (2H, m), 4.06 (5H, m), 4.16 (2H, m), 4.44-4.76 (4H, m), 5.34 (2H, m), 5.57-5.66 (1H, m), 6.13-6.25 (1H, m), 7.08 (2H, m). MS [ESI]: m/z: [M+H] calc. 889.6 obs. 889.7.

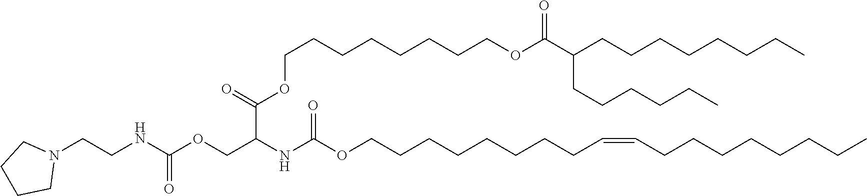 [SMILES: CCCCCCCC/C=C\CCCCCCCCOC(=O)NC(COC(=O)NCCN1CCCC1)C(=O)OCCCCCCCCOC(=O)C(CCCCCC)CCCCCCCC]

((Z)-8-((N-((octadec-9-en-1-yloxy)carbonyl)-O-((2-(pyrrolidin-1-yl)ethyl)carbamoyl)seryl)oxy)octyl 2-hexyldecanoate was synthesized according to representative synthetic scheme 2 and the corresponding methods. ¹H NMR (400 MHz, CDCl₃): δ 0.87 (9H, m), 1.25 (50H, m), 1.61 (10H, m), 1.82 (4H, m), 2.01 (4H, m), 2.30 (1H, m), 2.60 (6H, m), 3.30 (2H, m), 4.05 (4H, t, J=6.66 Hz), 4.15 (2H, t, J=6.86 Hz), 4.30-4.48 (2H, m), 4.48-4.59 (1H, m), 5.29 (1H, s), 5.34 (2H, m), 5.53-5.60 (1H, m). MS [ESI]: m/z: [M+H] calc. 906.7 obs. 906.8.

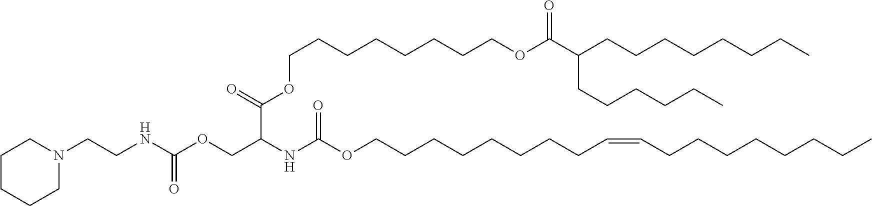 [SMILES: CCCCCCCC/C=C\CCCCCCCCOC(=O)NC(COC(=O)NCCN1CCCCC1)C(=O)OCCCCCCCCOC(=O)C(CCCCCC)CCCCCCCC]

(Z)-8-((N-((octadec-9-en-1-yloxy)carbonyl)-O-((2-(piperidin-1-yl)ethyl)carbamoyl)seryl)oxy)octyl 2-hexyldecanoate was synthesized according to representative synthetic scheme 2 and the corresponding methods. ¹H NMR (400 MHz, CDCl₃): δ 0.87 (9H, m), 1.26 (50H, m), 1.38-1.49 (4H, m), 1.50-1.71 (12H, m), 2.04 (5H, m), 2.24-2.44 (6H, m), 3.24 (2H, m), 4.06 (6H, m), 4.28-4.49 (2H, m), 4.50-4.60 (1H, m), 5.21-5.28 (1H, m), 5.30 (1H, s), 5.34 (1H, m), 5.54 (1H, d, J=8.37 Hz). MS [ESI]: m/z: [M+H] calc. 920.7 obs. 920.8.

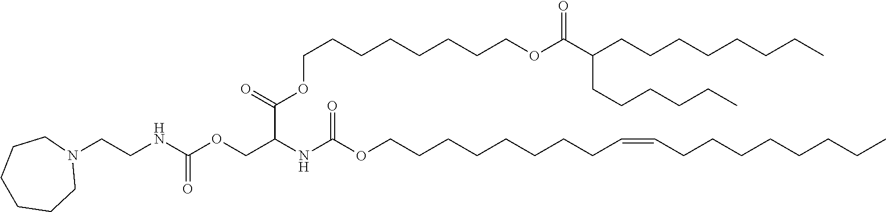 [SMILES: CCCCCCCC/C=C\CCCCCCCCOC(=O)NC(COC(=O)NCCN1CCCCCC1)C(=O)OCCCCCCCCOC(=O)C(CCCCCC)CCCCCCCC]

((Z)-8-((O-((2-(azepan-1-yl)ethyl)carbamoyl)-N-((octadec-9-en-1-yloxy)carbonyl)seryl)oxy)octyl 2-hexyldecanoate was synthesized according to representative synthetic scheme 2 and the corresponding methods. ¹H NMR (400 MHz, CDCl₃): δ 0.87 (9H, m), 1.25 (52H, m), 1.61 (16H, m), 2.02 (4H, m), 2.30 (1H, m), 2.67 (6H, m), 3.25 (2H, m), 4.06 (4H, m), 4.15 (2H, t, J=6.75 Hz), 4.29-4.48 (2H, m), 4.49-4.60 (1H, m), 5.29 (1H, s), 5.34 (2H, m), 5.51-5.62 (1H, m). MS [ESI]: m/z: [M+H] calc. 934.7 obs. 934.9.

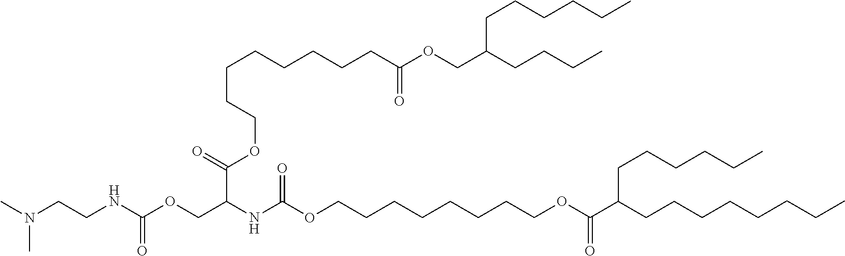 [SMILES: CCCCCCCCC(CCCCCC)C(=O)OCCCCCCCCOC(=O)NC(COC(=O)NCCN(C)C)C(=O)OCCCCCCCCC(=O)OCC(CCCC)CCCCCC]

9-(((9-((2-butyloctyl)oxy)-9-oxononyl)oxy)carbonyl)-2-methyl-6,11-dioxo-7,12-dioxa-2,5,10-triazaicosan-20-yl 2-hexyldecanoate was synthesized according to representative synthetic scheme 2 and the corresponding methods. ¹H NMR (400 MHz, CDCl₃): δ 0.87 (12H, m), 1.25 (54H, m), 1.61 (11H, m), 2.29 (3H, t, J=7.60 Hz), 2.51-2.62 (6H, m), 2.72-2.89 (2H, m), 3.36-3.51 (2H, m), 3.97 (2H, d, J=5.81 Hz), 4.06 (4H, t, J=7.19 Hz), 4.14 (2H, t, J=6.80 Hz), 4.30-4.50 (2H, m), 4.51-4.59 (1H, m), 5.71-5.82 (1H, m). MS [ESI]: m/z: [M+H] calc. 954.8 obs. 954.8.

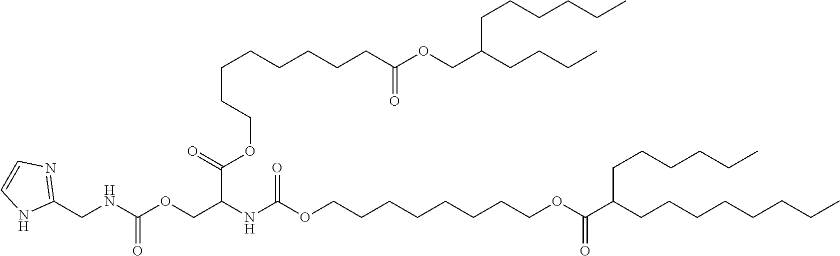 [SMILES: CCCCCCCCC(CCCCCC)C(=O)OCCCCCCCCOC(=O)NC(COC(=O)NCc1ncc[nH]1)C(=O)OCCCCCCCCC(=O)OCC(CCCC)CCCCCC]

12-(((((1H-imidazol-2-yl)methyl)carbamoyl)oxy)methyl)-26-butyl-10,13,23-trioxo-9,14,24-trioxa-11-azadotriacontyl 2-hexyldecanoate was synthesized according to representative synthetic scheme 2 and the corresponding methods. ¹H NMR (400 MHz, CDCl₃): δ 0.88 (12H, m), 1.25 (54H, m), 1.61 (11H, m), 2.30 (3H, t, J=7.39 Hz), 3.97 (211, d, J=5.81 Hz), 4.06 (4H, t, J=6.62 Hz), 4.15 (3H, t, J=7.65 Hz), 4.24-4.35 (1H, m), 4.40-4.73 (4H, m), 5.54-5.65 (1H, m), 5.91-6.00 (1H, m), 7.03 (2H, m). MS [ESI]: m/z: [M+H] calc. 963.7 obs. 963.7.

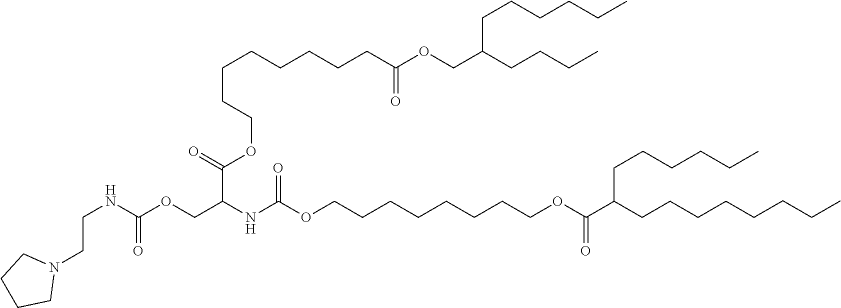 [SMILES: CCCCCCCCC(CCCCCC)C(=O)OCCCCCCCCOC(=O)NC(COC(=O)NCCN1CCCC1)C(=O)OCCCCCCCCC(=O)OCC(CCCC)CCCCCC]

26-butyl-10,13,23-trioxo-12-((((2-(pyrrolidin-1-yl)ethyl)carbamoyl)oxy)methyl)-9,14,24-trioxa-11-azadotriacontyl 2-hexyldecanoate was synthesized according to representative synthetic scheme 2 and the corresponding methods. ¹H NMR (400 MHz, CDCl₃): δ 0.87 (12H, m), 1.25 (54H, m), 1.62 (11H, m), 1.88 (4H, br s), 2.04 (1H, s), 2.29 (3H, t, J=7.76 Hz), 2.74 (5H, s), 3.36 (2H, m), 3.96 (2H, d, J=5.81 Hz), 4.06 (4H, t, J=6.86 Hz), 4.15 (2H, t, J=7.28 Hz), 4.30-4.48 (2H, m), 4.50-4.61 (1H, m), 5.56-5.69 (1H, m). MS [ESI]: m/z: [M+H] calc. 980.8 obs. 981.7.

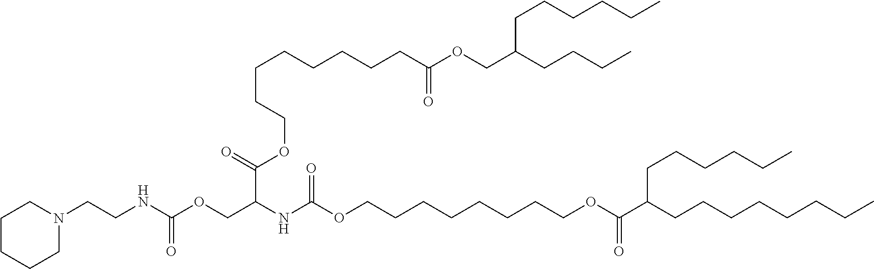 [SMILES: CCCCCCCCC(CCCCCC)C(=O)OCCCCCCCCOC(=O)NC(COC(=O)NCCN1CCCCC1)C(=O)OCCCCCCCCC(=O)OCC(CCCC)CCCCCC]

26-butyl-10,13,23-trioxo-12-((((2-(piperidin-1-yl)ethyl)carbamoyl)oxy)methyl)-9,14,24-trioxa-11-azadotriacontyl 2-hexyldecanoate was synthesized according to representative synthetic scheme 2 and the corresponding methods. 1H NMR (400 MHz, CDCl₃): δ 0.87 (12H, m), 1.25 (54H, m), 1.61 (17H, m), 2.29 (3H, t, J=7.93 Hz), 2.36-2.76 (6H, m), 3.26-3.44 (2H, m), 3.97 (2H, d, J=5.81 Hz), 4.06 (4H, t, J=6.86 Hz), 4.15 (2H, t, J=6.71 Hz), 4.30-4.49 (2H, m), 4.54 (111, m), 5.52-5.67 (1H, m). MS [ESI]: m/z: [M+H] calc. 994.8 obs. 994.6.

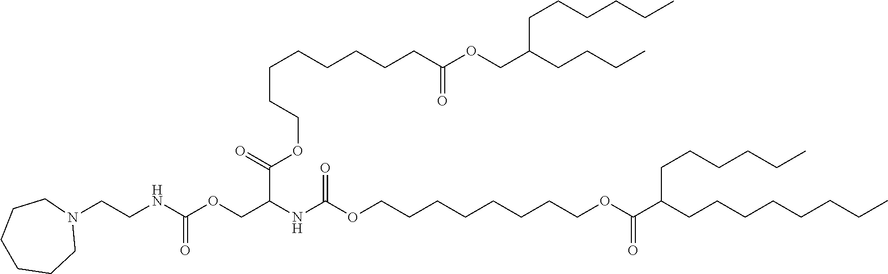 [SMILES: CCCCCCCCC(CCCCCC)C(=O)OCCCCCCCCOC(=O)NC(COC(=O)NCCN1CCCCCC1)C(=O)OCCCCCCCCC(=O)OCC(CCCC)CCCCCC]

((Z)-8-((O-((2-(azepan-1-yl)ethyl)carbamoyl)-N-((octadec-9-en-1-yloxy)carbonyl)seryl)oxy)octyl 2-hexyldecanoate was synthesized according to representative synthetic scheme 2 and the corresponding methods. 1H NMR (400 MHz, CDCl₃): δ 0.87 (12H, m), 1.25 (52H, m), 1.61 (21H, m), 2.29 (3H, t, J=7.62 Hz), 2.48-3.01 (6H, m), 3.25-3.44 (2H, m), 3.97 (2H, d, J=5.81 Hz), 4.06 (4H, t, J=6.76 Hz), 4.15 (2H, t, J=7.17 Hz), 4.29-4.49 (211, m), 4.50-4.61 (1H, m), 5.53-5.69 (1H, m). MS [ESI]: m/z: [M+H] calc. 1008.8 obs. 1008.7.

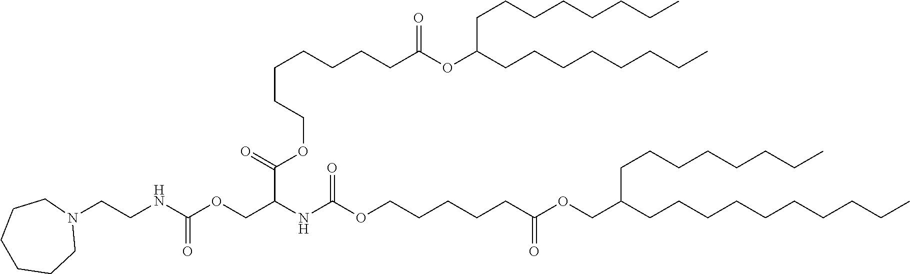 [SMILES: CCCCCCCCCCC(CCCCCCCC)COC(=O)CCCCCOC(=O)NC(COC(=O)NCCN1CCCCCC1)C(=O)OCCCCCCCC(=O)OC(CCCCCCCC)CCCCCCCC]

2-octyldodecyl 1-(azepan-1-yl)-7-(((8-(heptadecan-9-yloxy)-8-oxooctyl)oxy)carbonyl)-4,9-dioxo-5,10-dioxa-3,8-diazahexadecan-16-oate was synthesized according to representative synthetic scheme 2 and the corresponding methods. ¹H NMR (400 MHz, CDCl₃): δ 0.88 (12H, t, J=6.70 Hz), 1.26 (61H, m), 1.45-1.54 (4H, m), 1.54-1.71 (20H, m), 2.04 (2H, s), 2.29 (4H, quint, J=7.62 Hz), 2.52-2.65 (4H, m), 3.20 (2H, d, J=6.66 Hz), 3.96 (211, d, J=5.72 Hz), 4.13 (5H, m), 4.29-4.48 (211, m), 4.49-4.58 (1H, m), 4.86 (111, quint, J=6.75 Hz), 5.56 (1H, d, J=8.28 Hz). MS [ESI]: m/z: [M+H] calc. 1092.9 obs. 1093.0.

### Experimental Example 1

### Materials and Methods

1,2-distearoyl-sn-glycero-3-phosphocholine (DSPC), Cholesterol, 1,2-Dimyristoyl-rac-glycero-3-methoxypolyethylene glycol-2000 (DMG-PEG 2000) were from Avanti Lipids. The Quant-it™ RiboGreen RNA Assay Kit was from ThermoFisher Scientific. The in-vitro Luciferase Assay System, CellTiter-Glo® Luminescent Cell Viability Assay and lysis buffers were from Promega. Cytokine kit ProcartaPlex Mouse 2-plex (MCP-1 and IL-6) was from Thermo Fisher Scientific. The hEPO ELISA kit was from STEMCELL Technologies.

### Lipid Nanoparticles (LNPs) Preparations

Ionizable lipid, DSPC, cholesterol and DMG-PEG2000 were mixed in absolute ethanol, at a specific molar ratio listed in the Table. Circular RNA payloads were suspended in 25 mM Acetate Buffer (pH 4.5). To form LNPs, three volumes of luciferase encoding cirRNA (SEQ ID NO:1), were mixed with one volume of lipids in ethanol solution by microfluidic mixing with the the INano™ L+(Micro& Nano) at a flow rate of 12 ml/min. The molar ratio of ionizable lipid to ribonucleotide was 6. After encapsulation, the particles were dialyzed twice against PBS. The LNP formulation was concentrated to targeted RNA concentration by Amicon® Ultra Centrifugal Filter (100 kDa MWCO) and filtered by a 0.65/0.22 um sterile filter.

| Formulation | | |
|---|---|---|
| No. | Lipid composition | N/P |
| 001 | Ionizable lipid:DSPC:cholesterol:DMG-PEG2000 = | 6 |
| | 50:10:38.5:1.5 | |
| 010 | Ionizable lipid:DSPC:cholesterol:DSPE-PEG2000 = | 6 |
| | 38:30:30:2 | |

### Physiochemical Characteristics

The LNP size and polydispersity index (PDI), determined by dynamic light scattering (DLS), and zeta potential were measured by a Zetasizer machine (Malvern). RNA concentration and encapsulation efficacy (EE %) were analyzed by the Quant-IT Ribogreen kit by calculating the percentage encapsulation at 100%—(RNA-LNPs/RNA-LNPs with Triton X-100).

| Formulation No. | Lipid No. | RNA No. | Size (nm) | PDI | EE (%) |
|---|---|---|---|---|---|
| 010 | 549 | 331 | 57.35 | 0.13 | 96.68 |
| 010 | 550 | 331 | 67.81 | 0.13 | 94.88 |
| 010 | 601 | 331 | 62.33 | 0.08 | 95.98 |
| 010 | 602 | 331 | 64.73 | 0.16 | 97.13 |
| 010 | 700 | 331 | 55.29 | 0.07 | 96.73 |
| 010 | 701 | 331 | 60.60 | 0.07 | 95.09 |
| 010 | 728 | 331 | 56.88 | 0.09 | 96.83 |
| 010 | 729 | 331 | 57.90 | 0.10 | 95.94 |
| 010 | 730 | 331 | 59.50 | 0.09 | 96.20 |

### In Vitro Expression

2E5 Hep G2 cells were transfected with 100 ng/mL of RNA-LNPs encoding for firefly-luciferase. At 72 hours post transfection, the cells were washed and lysed. The luciferase expression (2-well repeat) was determined in the cell lysates with the luciferase assay reagent kit (Promega), according to the kit protocol, luciferase activity was measured by a luminometer.

### In Vitro Toxicity

2E5 Hep G2 cells were transfected with 1 μg/mL RNA-LNPs. At 72 hours post transfection, cell viability (2-well repeat) was determined with the CellTiter-Glo® reagent kit, according to the kit protocol, measured by a luminometer. Cell only as control (100%).

The cell viability was calculated according to the following formula:

$$\frac{transfected cells luminescence signal}{untreated cells luminescence signal} \times 100 \%$$

| | | | In vitro | In vitro cell |
|---|---|---|---|---|
| Formulation | Lipid | RNA | Expression | viability (%) |
| No. | No. | No. | (Mean) | (Mean) |
| 010 | 549 | 331 | 5.75E+05 | 88.89 |
| 010 | 550 | 331 | 5.74E+05 | 87.47 |
| 010 | 601 | 331 | 6.24E+05 | 98.14 |
| 010 | 602 | 331 | 6.30E+05 | 84.17 |
| 010 | 700 | 331 | 3.61E+05 | 99.10 |
| 010 | 701 | 331 | 3.96E+05 | 96.43 |
| 010 | 728 | 331 | 4.71E+05 | 90.20 |
| 010 | 729 | 331 | 3.44E+05 | 80.55 |
| 010 | 730 | 331 | 7.72E+05 | 98.64 |

### In Vivo Expression by IV

7-8 week old, female Balb/c mice (N=3) were injected with 0.05 mg*kg−1 of RNA-LNPs encoding for firefly-luciferase by intravenous injection. At 24- and 72-hours post injection, animals were intraperitoneally injected with 15 mg/mi of Luciferin substrate and whole-body luminescence was determined by a bioluminescent in vivo imaging system (IVIS).

| | | | Whole body | Whole body |
|---|---|---|---|---|
| Formulation | Lipid | RNA | at 24 h (p/s) | at 72 h (p/s) |
| No. | No. | No. | (Mean) | (Mean) |
| 010 | 549 | 331 | 2.75E+08 | 7.85E+07 |
| 010 | 550 | 331 | 2.36E+08 | 4.96E+07 |
| 010 | 601 | 331 | 3.82E+08 | 8.02E+07 |
| 010 | 602 | 331 | 2.66E+08 | 1.24E+08 |
| 010 | 700 | 331 | 3.40E+08 | 1.34E+08 |
| 010 | 701 | 331 | 4.00E+08 | 1.08E+08 |
| 010 | 728 | 331 | 1.86E+08 | 5.78E+07 |
| 010 | 729 | 331 | 1.86E+08 | 4.99E+07 |
| 010 | 730 | 331 | 2.24E+08 | 8.49E+07 |

### Physiochemical Characteristics

The LNP size and polydispersity index (PDI), determined by dynamic light scattering (DLS), and zeta potential were measured by a Zetasizer machine (Malvern). RNA concentration and encapsulation efficacy (EE %) were analyzed by the Quant-IT Ribogreen kit by calculating the percentage encapsulation at 100%—(RNA-LNPs/RNA-LNPs with Triton X-100).

| Formulation | Lipid | RNA | Size | | EE |
|---|---|---|---|---|---|
| No. | No. | No. | (nm) | PDI | (%) |
| 001 | 736 | 331 | 66.29 | 0.01 | 91.61 |
| 001 | 792 | 331 | 64.18 | 0.02 | 92.11 |
| 001 | 858 | 331 | 63.50 | 0.04 | 91.60 |
| 001 | 881 | 331 | 61.73 | 0.02 | 90.26 |
| 001 | 883 | 331 | 69.88 | 0.05 | 96.96 |
| 001 | 888 | 331 | 62.46 | 0.01 | 93.81 |
| 001 | 889 | 331 | 61.64 | 0.02 | 95.41 |
| 001 | 897 | 331 | 60.19 | 0.04 | 95.30 |
| 001 | 907 | 331 | 83.82 | 0.02 | 98.30 |
| 001 | 909 | 331 | 74.26 | 0.02 | 98.41 |
| 001 | 912 | 331 | 74.18 | 0.03 | 97.83 |
| 001 | 930 | 331 | 92.24 | 0.04 | 97.46 |

### In Vitro Expression

2E5 Hep G2 cells were transfected with 100 ng/mL of RNA-LNPs encoding for firefly-luciferase. At 72 hours post transfection, the cells were washed and lysed. The luciferase expression (2-well repeat) was determined in the cell lysates with the luciferase assay reagent kit (Promega), according to the kit protocol, luciferase activity was measured by a luminometer.

### In Vitro Toxicity

2E5 Hep G2 cells were transfected with 1 μg/mL RNA-LNPs. At 72 hours post transfection, cell viability (2-well repeat) was determined with the CellTiter-Glo® reagent kit, according to the kit protocol, measured by a luminometer. Cell only as control (100%).

The cell viability was calculated according to the following formula:

$$\frac{transfected cells luminescence signal}{untreated cells luminescence signal} \times 100 \%$$

| | | | In vitro | In vitro cell |
|---|---|---|---|---|
| Formulation | Lipid | RNA | Expression | viability (%) |
| No. | No. | No. | (Mean) | (Mean) |
| 001 | 736 | 331 | 7.05E+05 | 73.49 |
| 001 | 792 | 331 | 8.10E+05 | 81.74 |
| 001 | 858 | 331 | 2.88E+05 | 92.44 |
| 001 | 881 | 331 | 4.02E+05 | 86.94 |
| 001 | 883 | 331 | 2.82E+05 | 93.23 |
| 001 | 888 | 331 | 4.84E+05 | 89.73 |
| 001 | 889 | 331 | 2.46E+05 | 91.21 |
| 001 | 897 | 331 | 1.77E+05 | 88.39 |
| 001 | 907 | 331 | 2.56E+04 | 97.33 |
| 001 | 909 | 331 | 7.29E+04 | 94.80 |
| 001 | 912 | 331 | 4.57E+04 | 92.96 |
| 001 | 930 | 331 | 8.27E+03 | 92.08 |

### In Vivo Expression by IV

7-8 week old, female Balb/c mice (N=3) were injected with 0.05 mg*kg−1 of RNA-LNPs encoding for firefly-luciferase by intravenous injection. At 24-hours post injection, animals were intraperitoneally injected with 15 mg/ml of Luciferin substrate and whole-body/liver/spleen luminescence was determined by a bioluminescent in vivo imaging system (IVIS).

| | | | Whole body | Liver | Spleen |
|---|---|---|---|---|---|
| Formulation | Lipid | RNA | (p/s) | (p/s) | (p/s) |
| No. | No. | No. | (Mean) | (Mean) | (Mean) |
| 001 | 736 | 331 | 2.08E+09 | 2.87E+08 | 1.39E+08 |
| 001 | 792 | 331 | 2.18E+09 | 2.90E+08 | 1.01E+08 |
| 001 | 858 | 331 | 8.74E+08 | 3.71E+08 | 6.61E+07 |
| 001 | 881 | 331 | 1.18E+09 | 2.25E+08 | 6.14E+07 |
| 001 | 883 | 331 | 1.60E+09 | 1.59E+08 | 2.09E+07 |
| 001 | 888 | 331 | 1.35E+09 | 2.18E+08 | 7.61E+07 |
| 001 | 889 | 331 | 1.04E+09 | 1.70E+08 | 8.56E+07 |
| 001 | 897 | 331 | 8.36E+08 | 1.67E+08 | 7.67E+07 |
| 001 | 907 | 331 | 1.18E+09 | 1.46E+08 | 3.43E+07 |
| 001 | 909 | 331 | 7.25E+08 | 7.57E+07 | 2.35E+07 |
| 001 | 912 | 331 | 8.26E+07 | 1.67E+07 | 7.97E+06 |
| 001 | 930 | 331 | 1.31E+08 | 3.49E+07 | 6.28E+06 |

### In Vivo Tolerability by IV

6-8 week old, male C57BL/6J mice (N=3) were injected with 1.0 mg*kg/1 of RNA-LNPs encoding for firefly-luciferase by intravenous injection. At 2- and 24-hours post injection, about 40 μL blood was harvested and immediately added to the procoagulant containing tube. Stand for 30 min at room temperature and then centrifuged at 2500 g×10 min to obtain serum. About 15 μL serum samples were aliquoted for cytokine (MCP-1 and IL-6) detection by Luminex (ProcartaPlex Mouse 2-plex, Thermo). PBS group as negative control.

| Formu- | | | MCP-1 | MCP-1 | IL-6 | IL-6 |
|---|---|---|---|---|---|---|
| lation | Lipid | RNA | at 2 h | at 24 h | at 2 h | at 24 h |
| No. | No. | No. | (pg/mL) | (pg/mL) | (pg/mL) | (pg/mL) |
| PBS | | | 6.00E+01 | 2.65E+01 | 2.04E+00 | 2.49E+00 |
| 001 | 736 | 331 | 3.83E+03 | 2.60E+02 | 4.42E+02 | 3.03E+00 |
| 001 | 792 | 331 | 1.62E+03 | 2.16E+02 | 2.59E+01 | 6.86E+00 |
| 001 | 858 | 331 | 1.36E+03 | 1.11E+02 | 2.30E+01 | 3.13E+00 |
| 001 | 881 | 331 | 1.19E+03 | 2.53E+02 | 1.28E+01 | 6.69E+00 |
| 001 | 883 | 331 | 1.86E+03 | 2.20E+02 | 1.64E+01 | 5.01E+00 |
| 001 | 888 | 331 | 3.96E+02 | 2.06E+02 | 5.50E+00 | 5.71E+00 |
| 001 | 889 | 331 | 7.60E+02 | 2.86E+02 | 7.62E+00 | 4.69E+00 |
| 001 | 907 | 331 | 1.02E+03 | 8.15E+01 | 1.40E+01 | 2.35E+00 |

### In Vivo Expression by IV

6-8 week old, male C57BL/6J mice (N=3) were injected with 1.0 mg*kg−1 of RNA-LNPs encoding for firefly-luciferase by intravenous injection. At 24-hours post injection, animals were intraperitoneally injected with 15 mg/ml of Luciferin substrate and whole-body/liver/spleen/kidney/small intestine/large intestine luminescence were determined by a bioluminescent in vivo imaging system (IVIS).

| | | | Whole | | | | Small | Large |
|---|---|---|---|---|---|---|---|---|
| Formulation | Lipid | RNA | body | Liver | Spleen | Kidney | intestine | intestine |
| No. | No. | No. | (p/s) | (p/s) | (p/s) | (p/s) | (p/s) | (p/s) |
| 001 | 736 | 331 | 5.28E+09 | 2.87E+09 | 3.38E+08 | 5.22E+07 | 6.48E+08 | 1.78E+08 |
| 001 | 792 | 331 | 2.76E+09 | 2.74E+09 | 3.20E+08 | 3.20E+07 | 5.01E+08 | 8.04E+07 |
| 001 | 858 | 331 | 1.92E+09 | 8.64E+08 | 2.33E+08 | 2.31E+07 | 6.45E+08 | 1.58E+08 |
| 001 | 881 | 331 | 2.92E+09 | 1.65E+09 | 2.37E+08 | 4.26E+07 | 8.57E+08 | 1.30E+08 |
| 001 | 883 | 331 | 1.65E+09 | 3.22E+09 | 2.19E+08 | 6.37E+06 | 1.46E+08 | 3.50E+07 |
| 001 | 888 | 331 | 4.10E+09 | 4.61E+09 | 2.55E+08 | 2.72E+07 | 8.59E+08 | 1.07E+08 |
| 001 | 889 | 331 | 3.48E+09 | 5.79E+09 | 2.95E+08 | 3.47E+07 | 1.04E+09 | 2.12E+08 |
| 001 | 907 | 331 | 8.99E+08 | 1.91E+08 | 5.98E+07 | 5.43E+06 | 3.03E+08 | 3.94E+07 |

### Physiochemical Characteristics

The LNP size and polydispersity index (PDI), determined by dynamic light scattering (DLS), and zeta potential were measured by a Zetasizer machine (Malvern). RNA concentration and encapsulation efficacy (EE %) were analyzed by the Quant-IT Ribogreen kit by calculating the percentage encapsulation at 100%—(RNA-LNPs/RNA-LNPs with Triton X-100).

| Formulation | Lipid | RNA | Size | | EE |
|---|---|---|---|---|---|
| No. | No. | No. | (nm) | PDI | (%) |
| 001 | 911 | 331 | 64.33 | 0.03 | 98.24 |
| 001 | 914 | 331 | 70.33 | 0.04 | 87.49 |
| 001 | 918 | 331 | 69.23 | 0.01 | 93.47 |
| 001 | 919 | 331 | 66.17 | 0.02 | 96.20 |
| 001 | 923 | 331 | 81.29 | 0.03 | 88.91 |
| 001 | 924 | 331 | 78.20 | 0.05 | 90.24 |
| 001 | 928 | 331 | 75.43 | 0.01 | 87.36 |
| 001 | 929 | 331 | 67.82 | 0.01 | 95.55 |
| 001 | 955 | 331 | 81.86 | 0.03 | 91.27 |
| 001 | 933 | 331 | 75.47 | 0.02 | 92.12 |
| 001 | 934 | 331 | 70.72 | 0.03 | 95.74 |
| 001 | 939 | 331 | 104.00 | 0.19 | 90.23 |
| 001 | 944 | 331 | 86.75 | 0.17 | 90.80 |
| 001 | 954 | 331 | 83.18 | 0.01 | 88.46 |
| 001 | 959 | 331 | 91.09 | 0.13 | 95.49 |

### In Vitro Expression

2E5 Hep G2 cells were transfected with 100 ng/mL of RNA-LNPs encoding for firefly-luciferase. At 72 hours post transfection, the cells were washed and lysed. The luciferase expression (2-well repeat) was determined in the cell lysates with the luciferase assay reagent kit (Promega), according to the kit protocol, luciferase activity was measured by a luminometer.

### In Vitro Toxicity

2E5 Hep G2 cells were transfected with 1 μg/mL RNA-LNPs. At 72 hours post transfection, cell viability (2-well repeat) was determined with the CellTiter-Glo® reagent kit, according to the kit protocol, measured by a luminometer. Cell only as control (100%).

The cell viability was calculated according to the following formula:

$$\frac{transfected cells luminescence signal}{untreated cells luminescence signal} \times 100 \%$$

| | | | In vitro | In vitro cell |
|---|---|---|---|---|
| Formulation | Lipid | RNA | Expression | viability (%) |
| No. | No. | No. | (Mean) | (Mean) |
| 001 | 911 | 331 | 5.01E+05 | 81.84 |
| 001 | 914 | 331 | 1.56E+06 | 79.98 |
| 001 | 918 | 331 | 1.25E+06 | 85.92 |
| 001 | 919 | 331 | 4.10E+05 | 82.59 |
| 001 | 923 | 331 | 7.34E+05 | 88.59 |
| 001 | 924 | 331 | 6.15E+05 | 89.34 |
| 001 | 928 | 331 | 4.72E+05 | 95.03 |
| 001 | 929 | 331 | 2.24E+05 | 100.37 |
| 001 | 955 | 331 | 2.53E+05 | 95.28 |
| 001 | 933 | 331 | 1.26E+06 | 95.05 |
| 001 | 934 | 331 | 5.28E+05 | 94.29 |
| 001 | 939 | 331 | 3.08E+05 | 95.74 |
| 001 | 944 | 331 | 3.60E+05 | 95.62 |
| 001 | 954 | 331 | 4.78E+05 | 93.41 |
| 001 | 959 | 331 | 2.95E+05 | 96.03 |

### In Vivo Expression by IV

7-8 week old, female Balb/c mice (N=3) were injected with 0.05 mg*kg−1 of RNA-LNPs encoding for firefly-luciferase by intravenous injection. At 24-hours post injection, animals were intraperitoneally injected with 15 mg/ml of Luciferin substrate and whole-body/liver/spleen luminescence was determined by a bioluminescent in vivo imaging system (IVIS).

| | | | Whole body | Liver | |
|---|---|---|---|---|---|
| Formulation | Lipid | RNA | (p/s) | (p/s) | Spleen(p/s) |
| No. | No. | No. | (Mean) | (Mean) | (Mean) |
| 001 | 911 | 331 | 1.17E+09 | 5.42E+08 | 1.27E+08 |
| 001 | 914 | 331 | 1.19E+09 | 3.77E+08 | 5.60E+07 |
| 001 | 918 | 331 | 1.21E+09 | 4.14E+08 | 7.74E+07 |
| 001 | 919 | 331 | 1.86E+09 | 5.47E+08 | 2.28E+08 |
| 001 | 923 | 331 | 1.57E+09 | 4.09E+08 | 5.03E+07 |
| 001 | 924 | 331 | 3.08E+09 | 9.02E+08 | 2.37E+08 |
| 001 | 928 | 331 | 9.65E+08 | 2.35E+08 | 4.80E+07 |
| 001 | 929 | 331 | 1.02E+09 | 2.99E+08 | 5.00E+07 |
| 001 | 955 | 331 | 2.15E+09 | 7.00E+08 | 9.11E+07 |
| 001 | 933 | 331 | 2.89E+09 | 6.92E+08 | 1.49E+08 |
| 001 | 934 | 331 | 1.87E+09 | 6.73E+08 | 2.04E+08 |
| 001 | 939 | 331 | 1.17E+09 | 2.92E+08 | 1.74E+08 |
| 001 | 944 | 331 | 1.29E+09 | 5.10E+08 | 1.31E+08 |
| 001 | 954 | 331 | 2.71E+09 | 8.01E+08 | 9.82E+07 |
| 001 | 959 | 331 | 3.16E+09 | 1.07E+09 | 2.04E+08 |

### In Vivo Tolerability by IV

6-8 week old, male C57BL/6J mice (N=3) were injected with 1.0 mg*kg/1 of RNA-LNPs encoding for firefly-luciferase by intravenous injection. At 2- and 24-hours post injection, about 40 μL blood was harvested and immediately added to the procoagulant containing tube. Stand for 30 min at room temperature and then centrifuged at 2500 g×10 min to obtain serum. About 15 μL serum samples were aliquoted for cytokine (MCP-1 and IL-6) detection by Luminex (ProcartaPlex Mouse 2-plex, Thermo). PBS group as negative control.

| Formu- | | | MCP-1 | MCP-1 | IL-6 | IL-6 |
|---|---|---|---|---|---|---|
| lation | Lipid | RNA | at 2 h | at 24 h | at 2 h | at 24 h |
| No. | No. | No. | (pg/mL) | (pg/mL) | (pg/mL) | (pg/mL) |
| PBS | | | 3.05E+01 | 2.89E+01 | 7.43E+00 | 3.45E+00 |
| 001 | 911 | 331 | 2.00E+03 | 2.54E+02 | 2.85E+01 | 4.43E+00 |
| 001 | 914 | 331 | 4.52E+02 | 1.48E+02 | 4.60E+00 | 2.77E+00 |
| 001 | 918 | 331 | 8.80E+02 | 3.00E+02 | 9.30E+00 | 4.73E+00 |
| 001 | 919 | 331 | 1.29E+03 | 2.56E+02 | 2.58E+01 | 5.07E+00 |
| 001 | 923 | 331 | 6.39E+02 | 7.50E+01 | 1.19E+01 | 2.37E+00 |
| 001 | 924 | 331 | 1.39E+03 | 2.07E+02 | 4.71E+01 | 5.90E+00 |
| 001 | 928 | 331 | 5.74E+02 | 7.49E+01 | 1.19E+01 | 3.05E+01 |
| 001 | 929 | 331 | 7.83E+02 | 5.86E+01 | 1.47E+01 | 2.10E+00 |

### In Vivo Tolerability by IV

6-8 week old, male C57BL/6J mice (N=3) were injected with 1.0 mg*kg/1 of RNA-LNPs encoding for firefly-luciferase by intravenous injection. At 2- and 24-hours post injection, about 40 μL blood was harvested and immediately added to the procoagulant containing tube. Stand for 30 min at room temperature and then centrifuged at 2500 g×10 min to obtain serum. About 15 μL serum samples were aliquoted for cytokine (MCP-1 and IL-6) detection by Luminex (ProcartaPlex Mouse 2-plex, Thermo). PBS group as negative control.

| Formu- | | | MCP-1 | MCP-1 | IL-6 | IL-6 |
|---|---|---|---|---|---|---|
| lation | Lipid | RNA | at 2 h | at 24 h | at 2 h | at 24 h |
| No. | No. | No. | (pg/mL) | (pg/mL) | (pg/mL) | (pg/mL) |
| PBS | | | 2.54E+01 | 4.62E+01 | 7.40E+00 | 2.80E+00 |
| 001 | 955 | 331 | 3.35E+03 | 5.80E+02 | 1.14E+03 | 7.13E+00 |
| 001 | 933 | 331 | 1.24E+03 | 3.00E+02 | 6.84E+01 | 8.03E+00 |
| 001 | 934 | 331 | 1.36E+03 | 2.29E+02 | 4.07E+01 | 7.83E+00 |
| 001 | 939 | 331 | 2.44E+03 | 9.56E+02 | 4.28E+03 | 9.43E+00 |
| 001 | 944 | 331 | 3.64E+03 | 1.61E+02 | 4.32E+02 | 2.50E+00 |
| 001 | 954 | 331 | 4.78E+03 | 7.89E+02 | 4.22E+03 | 8.87E+00 |
| 001 | 959 | 331 | 1.76E+03 | 1.76E+02 | 7.87E+01 | 1.80E+00 |

### In Vivo Expression by IV

6-8 week old, male C57BL/6J mice (N=3) were injected with 1.0 mg*kg−1 of RNA-LNPs encoding for firefly-luciferase by intravenous injection. At 24-hours post injection, animals were intraperitoneally injected with 15 mg/nil of Luciferin substrate and whole-body/liver/spleen/kidney/small intestine/large intestine luminescence were determined by a bioluminescent in vivo imaging system (IVIS).

| | | | Whole | | | | Small | Large |
|---|---|---|---|---|---|---|---|---|
| Formulation | Lipid | RNA | body | Liver | Spleen | Kidney | intestine | intestine |
| No. | No. | No. | (p/s) | (p/s) | (p/s) | (p/s) | (p/s) | (p/s) |
| 001 | 911 | 331 | 7.27E+09 | 7.97E+09 | 8.51E+08 | 3.75E+07 | 6.47E+08 | 8.42E+07 |
| 001 | 914 | 331 | 2.46E+09 | 1.75E+09 | 2.11E+08 | 1.72E+07 | 4.42E+08 | 4.60E+07 |
| 001 | 918 | 331 | 5.08E+09 | 4.67E+09 | 3.95E+08 | 1.99E+07 | 6.30E+08 | 8.43E+07 |
| 001 | 919 | 331 | 6.01E+09 | 3.54E+09 | 6.74E+08 | 1.88E+07 | 6.16E+08 | 1.29E+08 |
| 001 | 923 | 331 | 2.42E+09 | 1.44E+09 | 3.85E+08 | 5.13E+06 | 3.01E+08 | 5.94E+07 |
| 001 | 924 | 331 | 4.40E+09 | 5.23E+09 | 1.13E+09 | 3.17E+07 | 7.18E+08 | 3.25E+08 |
| 001 | 928 | 331 | 2.17E+09 | 2.41E+09 | 3.25E+08 | 8.93E+06 | 1.61E+08 | 2.23E+07 |
| 001 | 929 | 331 | 2.83E+09 | 3.67E+09 | 4.12E+08 | 2.14E+07 | 3.16E+08 | 9.97E+07 |

### In Vivo Expression by IV

6-8 week old, male C57BL/6J mice (N=3) were injected with 1.0 mg*kg−1 of RNA-LNPs encoding for firefly-luciferase by intravenous injection. At 24-hours post injection, animals were intraperitoneally injected with 15 mg/nil of Luciferin substrate and whole-body/liver/spleen/kidney/small intestine/large intestine luminescence were determined by a bioluminescent in vivo imaging system (IVIS).

| | | | Whole | | | | Small | Large |
|---|---|---|---|---|---|---|---|---|
| Formulation | Lipid | RNA | body | Liver | Spleen | Kidney | intestine | intestine |
| No. | No. | No. | (p/s) | (p/s) | (p/s) | (p/s) | (p/s) | (p/s) |
| 001 | 955 | 331 | 4.06E+09 | 1.98E+09 | 2.12E+09 | 4.80E+07 | 6.59E+08 | 3.44E+08 |
| 001 | 933 | 331 | 3.45E+09 | 3.90E+09 | 5.98E+08 | 6.08E+07 | 6.36E+08 | 9.52E+07 |
| 001 | 934 | 331 | 2.46E+09 | 1.82E+09 | 1.17E+09 | 4.10E+07 | 6.60E+08 | 1.17E+08 |
| 001 | 939 | 331 | 3.50E+09 | 5.86E+07 | 1.18E+08 | 1.21E+07 | 4.96E+08 | 1.70E+08 |
| 001 | 944 | 331 | 1.06E+09 | 5.27E+08 | 4.76E+08 | 1.54E+07 | 1.93E+08 | 5.53E+07 |
| 001 | 954 | 331 | 1.08E+10 | 3.01E+09 | 7.74E+08 | 1.53E+08 | 1.69E+09 | 9.03E+08 |
| 001 | 959 | 331 | 7.04E+08 | 1.10E+09 | 4.52E+08 | 1.10E+07 | 1.43E+08 | 3.10E+07 |

### Physiochemical Characteristics

The LNP size and polydispersity index (PDI), determined by dynamic light scattering (DLS), and zeta potential were measured by a Zetasizer machine (Malvern). RNA concentration and encapsulation efficacy (EE %) were analyzed by the Quant-IT Ribogreen kit by calculating the percentage encapsulation at 100%—(RNA-LNPs/RNA-LNPs with Triton X-100).

| Formulation | Lipid | RNA | Size | | |
|---|---|---|---|---|---|
| No. | No. | No. | (nm) | PDI | EE(%) |
| 001 | 1017 | 331 | 69.70 | 0.07 | 97.70 |
| 001 | 1019 | 331 | 68.78 | 0.10 | 97.11 |
| 001 | 1020 | 331 | 57.55 | 0.01 | 94.34 |
| 001 | 1021 | 331 | 57.55 | 0.05 | 93.55 |

### In Vivo Expression by IV

7-8 week old, female Balb/c mice (N=3) were injected with 0.05 mg*kg−1 of RNA-LNPs encoding for firefly-luciferase by intravenous injection. At 24-hours post injection, animals were intraperitoneally injected with 15 mg/ml of Luciferin substrate and whole-body/liver/spleen luminescence was determined by a bioluminescent in vivo imaging system (IVIS).

| | | | Whole body | Liver | |
|---|---|---|---|---|---|
| Formulation | Lipid | RNA | (p/s) | (p/s) | Spleen(p/s) |
| No. | No. | No. | (Mean) | (Mean) | (Mean) |
| 001 | 1017 | 331 | 2.45E+08 | 3.91E+07 | 1.30E+07 |
| 001 | 1019 | 331 | 2.46E+08 | 3.60E+07 | 2.21E+07 |
| 001 | 1020 | 331 | 4.91E+08 | 1.38E+08 | 2.67E+07 |
| 001 | 1021 | 331 | 8.26E+08 | 1.19E+08 | 5.32E+07 |

### In Vivo Tolerability by IV

6-8 week old, male C57BL/6J mice (N=3) were injected with 1.0 mg*kg−1 of RNA-LNPs encoding for firefly-luciferase by intravenous injection. At 2- and 24-hours post injection, about 40 μL blood was harvested and immediately added to the procoagulant containing tube. Stand for 30 min at room temperature and then centrifuged at 2500 g×10 min to obtain serum. About 15 μL serum samples were aliquoted for cytokine (MCP-1 and IL-6) detection by Luminex (ProcartaPlex Mouse 2-plex, Thermo). PBS group as negative control.

| Formu- | | | MCP-1 | MCP-1 | IL-6 | IL-6 |
|---|---|---|---|---|---|---|
| lation | Lipid | RNA | at 2 h | at 24 h | at 2 h | at 24 h |
| No. | No. | No. | (pg/mL) | (pg/mL) | (pg/mL) | (pg/mL) |
| PBS | | | 2.25E+01 | 3.32E+01 | 7.30E−01 | 8.44E−01 |
| 001 | 1017 | 331 | 2.43E+03 | 9.46E+01 | 1.28E+01 | 7.92E−01 |
| 001 | 1019 | 331 | 3.72E+03 | 4.01E+02 | 4.04E+02 | 2.02E+00 |
| 001 | 1020 | 331 | 1.93E+03 | 1.80E+02 | 9.05E+01 | 1.07E+00 |
| 001 | 1021 | 331 | 1.56E+03 | 1.66E+02 | 2.86E+01 | 6.39E−01 |

### In Vivo Expression by IV

6-8 week old, male C57BL/6J mice (N=3) were injected with 1.0 mg*kg−1 of RNA-LNPs encoding for firefly-luciferase by intravenous injection. At 24-hours post injection, animals were intraperitoneally injected with 15 mg/nil of Luciferin substrate and whole-body/liver/spleen/kidney/small intestine/large intestine luminescence were determined by a bioluminescent in vivo imaging system (IVIS).

| | | | Whole | | | | Small | Large |
|---|---|---|---|---|---|---|---|---|
| Formulation | Lipid | RNA | body | Liver | Spleen | Kidney | intestine | intestine |
| No. | No. | No. | (p/s) | (p/s) | (p/s) | (p/s) | (p/s) | (p/s) |
| 001 | 1017 | 331 | 1.13E+09 | 9.32E+08 | 4.32E+08 | 9.00E+06 | 1.93E+08 | 2.09E+07 |
| 001 | 1019 | 331 | 2.49E+09 | 1.20E+09 | 4.98E+08 | 1.46E+07 | 5.41E+08 | 2.12E+08 |
| 001 | 1020 | 331 | 9.25E+09 | 1.90E+10 | 1.14E+09 | 5.29E+07 | 1.62E+09 | 2.16E+08 |
| 001 | 1021 | 331 | 1.48E+10 | 1.88E+10 | 6.45E+08 | 5.69E+07 | 1.60E+09 | 1.91E+08 |

### Physiochemical Characteristics

The LNP size and polydispersity index (PDI), determined by dynamic light scattering (DLS), and zeta potential were measured by a Zetasizer machine (Malvern). RNA concentration and encapsulation efficacy (EE %) were analyzed by the Quant-IT Ribogreen kit by calculating the percentage encapsulation at 100%—(RNA-LNPs/RNA-LNPs with Triton X-100).

| Formulation | Lipid | RNA | Size | | |
|---|---|---|---|---|---|
| No. | No. | No. | (nm) | PDI | EE(%) |
| 001 | 1041 | 331 | 59.33 | 0.08 | 90.29 |
| 001 | 1072 | 331 | 52.67 | 0.04 | 96.51 |

### In Vivo Tolerability by IV

6-8 week old, male C57BL/6J mice (N=3) were injected with 1.0 mg*kg−1 of RNA-LNPs encoding for firefly-luciferase by intravenous injection. At 2- and 24-hours post injection, about 40 μL blood was harvested and immediately added to the procoagulant containing tube. Stand for 30 min at room temperature and then centrifuged at 2500 g×10 min to obtain serum. About 15 μL serum samples were aliquoted for cytokine (MCP-1 and IL-6) detection by Luminex (ProcartaPlex Mouse 2-plex, Thermo). PBS group as negative control.

| Formu- | | | MCP-1 | MCP-1 | IL-6 | IL-6 |
|---|---|---|---|---|---|---|
| lation | Lipid | RNA | at 2 h | at 24 h | at 2 h | at 24 h |
| No. | No. | No. | (pg/mL) | (pg/mL) | (pg/mL) | (pg/mL) |
| PBS | | | 1.12E+01 | 1.36E+01 | 4.30E−01 | 4.10E−01 |
| 001 | 1041 | 331 | 1.18E+03 | 6.81E+01 | 5.12E+01 | 4.53E+00 |
| 001 | 1072 | 331 | 5.54E+02 | 3.51E+01 | 2.07E+00 | 9.47E−01 |

### In Vivo Expression by IV

6-8 week old, male C57BL/6J mice (N=3) were injected with 1.0 mg*kg−1 of RNA-LNPs encoding for firefly-luciferase by intravenous injection. At 24-hours post injection, animals were intraperitoneally injected with 15 mg/ml of Luciferin substrate and whole-body/liver/spleen/kidney/small intestine/large intestine luminescence were determined by a bioluminescent in vivo imaging system (IVIS).

| | | | Whole | | | | Small | Large |
|---|---|---|---|---|---|---|---|---|
| Formulation | Lipid | RNA | body | Liver | Spleen | Kidney | intestine | intestine |
| No. | No. | No. | (p/s) | (p/s) | (p/s) | (p/s) | (p/s) | (p/s) |
| 001 | 1041 | 331 | 8.03E+08 | 1.08E+09 | 1.93E+08 | 6.14E+06 | 1.96E+08 | 1.74E+07 |
| 001 | 1072 | 331 | 6.81E+09 | 1.40E+10 | 1.95E+08 | 8.41E+06 | 2.07E+08 | 1.22E+07 |

### Physiochemical Characteristics

The LNP size and polydispersity index (PDI), determined by dynamic light scattering (DLS), and zeta potential were measured by a Zetasizer machine (Malvern). RNA concentration and encapsulation efficacy (EE %) were analyzed by the Quant-IT Ribogreen kit by calculating the percentage encapsulation at 100%—(RNA-LNPs/RNA-LNPs with Triton X-100).

| Formulation | Lipid | RNA | Size | | EE |
|---|---|---|---|---|---|
| No. | No. | No. | (nm) | PDI | (%) |
| 001 | 549 | 331 | 61.34 | 0.04 | 94.58 |
| 001 | 550 | 331 | 72.66 | 0.15 | 94.50 |
| 001 | 601 | 331 | 80.16 | 0.09 | 95.30 |
| 001 | 602 | 331 | 76.34 | 0.08 | 97.94 |
| 001 | 700 | 331 | 62.57 | 0.06 | 94.45 |
| 001 | 701 | 331 | 76.05 | 0.10 | 92.16 |
| 001 | 728 | 331 | 65.33 | 0.05 | 96.53 |
| 001 | 729 | 331 | 71.29 | 0.08 | 95.62 |
| 001 | 730 | 331 | 76.53 | 0.05 | 97.08 |

### In Vitro Expression

2E5 Hep G2 cells were transfected with 100 ng/mL of RNA-LNPs encoding for firefly-luciferase. At 72 hours post transfection, the cells were washed and lysed. The luciferase expression (2-well repeat) was determined in the cell lysates with the luciferase assay reagent kit (Promega), according to the kit protocol, luciferase activity was measured by a luminometer.

### In Vitro Toxicity

2E5 Hep G2 cells were transfected with 1 μg/mL RNA-LNPs. At 72 hours post transfection, cell viability (2-well repeat) was determined with the CellTiter-Glo® reagent kit, according to the kit protocol, measured by a luminometer. Cell only as control (100%).

The cell viability was calculated according to the following formula:

$$\frac{transfected cells luminescence signal}{untreated cells luminescence signal} \times 100 \%$$

| | | | In vitro | In vitro cell |
|---|---|---|---|---|
| Formulation | Lipid | RNA | Expression | viability (%) |
| No. | No. | No. | (Mean) | (Mean) |
| 001 | 549 | 331 | 7.02E+05 | 79.09 |
| 001 | 550 | 331 | 1.89E+05 | 67.09 |
| 001 | 601 | 331 | 9.94E+05 | 81.18 |
| 001 | 602 | 331 | 5.82E+05 | 82.68 |
| 001 | 700 | 331 | 7.16E+05 | 78.82 |
| 001 | 701 | 331 | 6.00E+05 | 74.24 |
| 001 | 728 | 331 | 3.37E+05 | 71.13 |
| 001 | 729 | 331 | 1.64E+05 | 65.07 |
| 001 | 730 | 331 | 5.98E+05 | 75.29 |

### In Vivo Expression by ID

7-8 week old, female C57BL/6J mice (N=3) were injected with 0.15 mg*kg−1 of RNA-LNPs encoding for firefly-luciferase by intradermal injection. At 72-hours post injection, animals were intraperitoneally injected with 15 mg/mi of Luciferin substrate and whole-body/injection site skin/lymph node luminescence were determined by a bioluminescent in vivo imaging system (IVIS).

| | | | | Injection | |
|---|---|---|---|---|---|
| Formulation | Lipid | RNA | Whole body | site Skin | Lymph node |
| No. | No. | No. | (p/s) | (p/s) | (p/s) |
| 001 | 549 | 331 | 4.38E+07 | 3.41E+06 | 3.17E+06 |
| 001 | 550 | 331 | 3.09E+07 | 1.33E+07 | 2.06E+06 |
| 001 | 601 | 331 | 5.92E+07 | 9.69E+06 | 1.89E+06 |
| 001 | 602 | 331 | 1.40E+08 | 3.02E+07 | 3.01E+06 |
| 001 | 700 | 331 | 8.00E+07 | 1.75E+07 | 1.57E+06 |
| 001 | 701 | 331 | 6.46E+07 | 3.34E+07 | 6.94E+06 |
| 001 | 728 | 331 | 3.58E+07 | 4.04E+06 | 1.03E+06 |
| 001 | 729 | 331 | 6.76E+07 | 1.33E+07 | 1.42E+06 |
| 001 | 730 | 331 | 2.81E+07 | 1.63E+07 | 3.33E+06 |

### In Vivo Tolerability by IP

7-8 week old, female C57BL/6J mice (N=3) were injected with 0.15 mg*kg−1 of RNA-LNIPs encoding for firefly-luciferase by intradermal injection. At 72-hours post injection, injection site skin from each animal was collected in 10% formalin, for H&E staining. PBS group (N=2) as control. (Note: 1==minimal, 2=mild, 3=moderate, 4=marked, 5=severe; N=Normal; NA=Not applicable; —=No data)

| | | | Formulation No. | | | | | | | | | | | |
|---|---|---|---|---|---|---|---|---|---|---|---|---|---|---|
| | | | 001 | | | 001 | | | 001 | | | 001 | | |
| | | | Lipid No. | | | | | | | | | | | |
| | | | 549 | | | 550 | | | 601 | | | 602 | | |
| | | | RNA No. | | | | | | | | | | | |
| | PBS | | 331 | | | 331 | | | 331 | | | 331 | | |
| Animal No. | 1 | 2 | 1 | 2 | 3 | 1 | 2 | 3 | 1 | 2 | 3 | 1 | 2 | 3 |
| Tissue and Lesions | Grade | | | | | | | | | | | | | |
| Skin | N | N | N | — | — | — | — | — | — | — | — | — | — | — |
| Infiltration, mononuclear cells, | — | — | — | 1 | 1 | 1 | 1 | 1 | 1 | 1 | 1 | 1 | 1 | 1 |
| dermis/subcutaneous, focal | | | | | | | | | | | | | | |
| Edema, subcutaneous, focal | — | — | — | 1 | 1 | 1 | 1 | 1 | 1 | 1 | 1 | 1 | 1 | 2 |
| Erosion, focal | — | — | — | — | — | 1 | — | — | — | — | — | — | — | — |

| | | | | | |
|---|---|---|---|---|---|
| Total score | 0 | 4 | 7 | 6 | 7 |

| | Formulation No. | | | | | | | | | | | | | | |
|---|---|---|---|---|---|---|---|---|---|---|---|---|---|---|---|
| | 001 | | | 001 | | | 001 | | | 001 | | | 001 | | |
| | Lipid No. | | | | | | | | | | | | | | |
| | 700 | | | 701 | | | 728 | | | 729 | | | 730 | | |
| | RNA No. | | | | | | | | | | | | | | |
| | 331 | | | 331 | | | 331 | | | 331 | | | 331 | | |
| Animal No. | 1 | 2 | 3 | 1 | 2 | 3 | 1 | 2 | 3 | 1 | 2 | 3 | 1 | 2 | 3 |
| Tissue and Lesions | Grade | | | | | | | | | | | | | | |
| Skin | — | — | — | — | — | — | — | — | — | — | — | — | — | — | — |
| Infiltration, mononuclear cells, | 1 | 1 | 1 | 1 | 1 | 1 | 1 | 1 | 1 | 1 | 1 | 1 | 1 | 1 | 1 |
| dermis/subcutaneous, focal | | | | | | | | | | | | | | | |
| Edema, subcutaneous, focal | 1 | 1 | 2 | — | 1 | 1 | — | — | 1 | 1 | 1 | — | 1 | 1 | 1 |
| Erosion, focal | — | — | — | — | — | — | — | — | — | — | — | — | — | — | — |

| | | | | | |
|---|---|---|---|---|---|
| Total score | 7 | 5 | 4 | 5 | 6 |

### In Vivo Tolerability by IM

6-8 week old, male C57BL/6J mice (N=3) were injected with 1.0 mg*kg−1 of RNA-LNPs encoding for firefly-luciferase by intramuscular injection. At 2- and 24-hours post injection, about 40 μL blood was harvested and immediately added to the procoagulant containing tube. Stand for 30 min at room temperature and then centrifuged at 2500 g×10 min to obtain serum. About 15 μL serum samples were aliquoted for cytokine (MCP-1 and IL-6) detection by Luminex (ProcartaPlex Mouse 2-plex, Thermo). PBS group as negative control.

| Formu- | | | MCP-1 | MCP-1 | IL-6 | IL-6 |
|---|---|---|---|---|---|---|
| lation | Lipid | RNA | at 2 h | at 24 h | at 2 h | at 24 h |
| No. | No. | No. | (pg/mL) | (pg/mL) | (pg/mL) | (pg/mL) |
| PBS | | | 2.03E+02 | 8.37E+01 | 6.46E+00 | 2.67E+00 |
| 001 | 549 | 331 | 2.99E+02 | 3.98E+03 | 2.70E+01 | 1.46E+02 |
| 001 | 700 | 331 | 2.81E+02 | 3.27E+03 | 1.50E+01 | 1.43E+02 |
| 001 | 701 | 331 | 6.28E+02 | 4.62E+03 | 2.12E+01 | 1.08E+02 |
| 001 | 728 | 331 | 4.21E+02 | 3.74E+03 | 2.43E+01 | 1.43E+02 |

### In Vivo Expression by IM

6-8 week old, male C57BL/6J mice (N=3) were injected with 1.0 mg*kg−1 of RNA-LNPs encoding for firefly-luciferase by intramuscular injection. At 24-hours post injection, animals were intraperitoneally injected with 15 mg/ml of Luciferin substrate and whole-body/injection site muscle/liver/spleen/kidney/heart/lung luminescence were determined by a bioluminescent in vivo imaging system (IVIS).

| | | | Whole | Injection | | | | | |
|---|---|---|---|---|---|---|---|---|---|
| Formulation | Lipid | RNA | body | site muscle | Liver | Spleen | Kidney | Heart | Lung |
| No. | No. | No. | (p/s) | (p/s) | (p/s) | (p/s) | (p/s) | (p/s) | (p/s) |
| 001 | 549 | 331 | 1.10E+09 | 2.75E+08 | 1.76E+08 | 6.26E+07 | 1.23E+07 | 8.09E+05 | 1.24E+06 |
| 001 | 700 | 331 | 5.65E+08 | 3.07E+08 | 8.73E+07 | 1.21E+07 | 9.03E+05 | 1.79E+05 | 3.11E+05 |
| 001 | 701 | 331 | 8.27E+08 | 5.32E+08 | 2.35E+08 | 1.24E+08 | 2.70E+07 | 9.37E+05 | 2.10E+06 |
| 001 | 728 | 331 | 6.11E+08 | 5.61E+08 | 7.48E+07 | 3.40E+07 | 1.42E+07 | 4.42E+05 | 1.56E+06 |

### Lipid Nanoparticles (LNPs) Preparations

Ionizable lipid, DSPC, cholesterol and DMG-PEG2000 were mixed in absolute ethanol, at a specific molar ratio listed in the Table. Circular RNA payloads were suspended in 25 mM Acetate Buffer (pH 4.5). To form LNPs, three volumes of human erythropoietin (hEPO) encoding cirRNA (SEQ ID NO:2), were mixed with one volume of lipids in ethanol solution by microfluidic mixing with the INano™ L+ (Micro& Nano) at a flow rate of 12 ml/min. The molar ratio of ionizable lipid to ribonucleotide was 6. After encapsulation, the particles were dialyzed twice against PBS. The LNP formulation was concentrated to targeted RNA concentration by Amicon® Ultra Centrifugal Filter (100 kDa MWCO) and filtered by a 0.65/0.22 um sterile filter.

| Formulation | | |
|---|---|---|
| No. | Lipid composition | N/P |
| 001 | Ionizable lipid:DSPC:cholesterol:DMG-PEG2000 = | 6 |
| | 50:10:38.5:1.5 | |
| 010 | Ionizable lipid:DSPC:cholesterol:DSPE-PEG2000 = | 6 |
| | 38:30:30:2 | |

### Physiochemical Characteristics

The LNP size and polydispersity index (PDI), determined by dynamic light scattering (DLS), and zeta potential were measured by a Zetasizer machine (Malvern). RNA concentration and encapsulation efficacy (EE %) were analyzed by the Quant-IT Ribogreen kit by calculating the percentage encapsulation at 100%—(RNA-LNPs/RNA-LNPs with Triton X-100).

| Formulation | Lipid | RNA | Size | | EE |
|---|---|---|---|---|---|
| No. | No. | No. | (nm) | PDI | (%) |
| 001 | 549 | 019 | 64.53 | 0.04 | 94.25 |
| 001 | 550 | 019 | 66.58 | 0.04 | 92.20 |
| 001 | 601 | 019 | 73.40 | 0.04 | 90.47 |
| 001 | 602 | 019 | 67.47 | 0.04 | 95.79 |

### In Vivo Expression by IM

6-8 week old, male C57BL/6J mice (N=3) were injected with 0.075 mg*kg−1 of RNA-LNPs encoding for hEPO by intramuscular injection. At 2-/6-/24-/48-/72 h-hours post injection, about 80 μL blood will be harvested and immediately added to the procoagulant containing tube. Stand for 30 min at room temperature and then centrifuged at 2500 g×10 min to obtain serum. About 30 μL serum samples will be aliquoted for hEPO concentration detection by ELISA kit (STEMCELL Technologies).

| | | | hEPO | hEPO | hEPO | hEPO | hEPO |
|---|---|---|---|---|---|---|---|
| Formu- | | | conc. | conc. | conc. | conc. | conc. |
| lation | Lipid | RNA | (ng/mL) | (ng/mL) | (ng/mL) | (ng/mL) | (ng/mL) |
| No. | No. | No. | at 2 h | at 6 h | at 24 h | at 48 h | at 72 h |
| 001 | 549 | 019 | 2.47 | 7.26 | 12.57 | 2.61 | 0.87 |
| 001 | 550 | 019 | 1.56 | 6.19 | 9.69 | 2.38 | 0.67 |
| 001 | 601 | 019 | 1.12 | 7.35 | 12.74 | 3.29 | 1.06 |
| 001 | 602 | 019 | 1.02 | 5.02 | 7.65 | 2.44 | 0.76 |

### Physiochemical Characteristics

The LNP size and polydispersity index (PDI), determined by dynamic light scattering (DLS), and zeta potential were measured by a Zetasizer machine (Malvern). RNA concentration and encapsulation efficacy (EE %) were analyzed by the Quant-IT Ribogreen kit by calculating the percentage encapsulation at 100%—(RNA-LNPs/RNA-LNPs with Triton X-100).

| Formulation | Lipid | RNA | Size | | EE |
|---|---|---|---|---|---|
| No. | No. | No. | (nm) | PDI | (%) |
| 001 | 700 | 019 | 68.99 | 0.06 | 96.00 |
| 001 | 888 | 019 | 59.39 | 0.05 | 97.48 |
| 001 | 911 | 019 | 58.16 | 0.05 | 94.15 |
| 001 | 924 | 019 | 87.04 | 0.16 | 90.41 |
| 001 | 955 | 019 | 90.32 | 0.14 | 93.99 |
| 001 | 934 | 019 | 70.30 | 0.08 | 93.44 |
| 001 | 1020 | 019 | 55.95 | 0.06 | 97.29 |
| 001 | 1021 | 019 | 57.55 | 0.04 | 96.60 |

In Vivo Lipid Metabolism by IV 7-8 week old, male Sprague-Dawley (SD) rats (N=3) were injected with 0.15 mg*kg−1 of RNA-LNPs encoding for hEPO by intravenous injection. At 1-/6-/96-/168-hours post injection, about 100 μL blood will be harvested and immediately added to the EDTAK2 containing tube. Place on wet ice and then centrifuged at 3000 g×5 min to obtain plasma. 30 μL plasma will be aliquoted to the 1.5 mL tubes. At terminal 6-/168-hours post injection, the rats will be euthanized with CO2, hemorrhaged immediately with cardiac puncture, respectively. The livers were harvested, weighed and washed with saline, then put into 50 ml centrifuge tube placed in liquid nitrogen, then transferred into wet ice, and finally stored at −80° C. immediately. Both plasma and liver samples were used for ionizable lipid concentration quantification by LC-MS/MS. The lower limit of quantitation (LLOQ) is 1 ng/mL in plasma or 11 ng/g in liver. BQL—Below the lower limit of quantitation (LLOQ).

| | Time (h) | Lipid 700 | | | Lipid 888 | | | Lipid 911 | | |
|---|---|---|---|---|---|---|---|---|---|---|
| Plasma | 1 | 1442 | 1272 | 1635 | 1635 | 1635 | 1440 | 5572 | 3130 | 7622 |
| (ng/mL) | 6 | 359 | 641 | 966 | 966 | 966 | 975 | 2.59 | 3.41 | 298 |
| | 96 | 5.84 | 4.4 | 28.6 | 28.6 | 28.6 | 12.9 | BQL | BQL | BQL |
| | 168 | 1.24 | BQL | 9.51 | 9.51 | 9.51 | 8.26 | BQL | BQL | BQL |

| | Time (h) | Lipid 924 | | | Lipid 955 | | | Lipid 934 | | |
|---|---|---|---|---|---|---|---|---|---|---|
| Plasma | 1 | 5572 | 3130 | 7622 | 6059 | 23590 | 15983 | 5944 | 9980 | 3316 |
| (ng/mL) | 6 | 2.59 | 3.41 | 298 | 17 | 11.9 | 22 | 8.59 | BQL | 691 |
| | 96 | BQL | BQL | BQL | BQL | BQL | BQL | BQL | BQL | BQL |
| | 168 | BQL | BQL | BQL | BQL | BQL | BQL | BQL | BQL | BQL |

| | Time | | | | | | |
|---|---|---|---|---|---|---|---|
| | (h) | Lipid 1020 | | | Lipid 1021 | | |
| Plasma | 1 | 2424 | 2412 | 2622 | 3284 | 2558 | 2938 |
| (ng/mL) | 6 | 1214 | 1241 | 1471 | 1479 | 976 | 1347 |
| | 96 | 33.6 | 31.2 | 26.1 | 23.9 | 5.16 | 9.41 |
| | 168 | 7.35 | 11.3 | 4.48 | 6.98 | BQL | 1.89 |

| | Time (h) | Lipid 700 | | | Lipid 888 | | | Lipid 911 | | |
|---|---|---|---|---|---|---|---|---|---|---|
| Liver | 6 | 26812 | 25720 | 25028 | 48815 | 31120 | 47483 | 29536 | 35435 | 29353 |
| (ng/g) | 168 | 696.6 | 191.6 | 236.8 | 1641 | 2277 | 2814 | 1331 | 618 | 890 |

| | Time (h) | Lipid 924 | | | Lipid 955 | | | Lipid 934 | | |
|---|---|---|---|---|---|---|---|---|---|---|
| Liver | 6 | 1409 | 1864 | 1834 | 668 | 703 | 396 | BQL | 14 | BQL |
| (ng/g) | 168 | BQL | BQL | BQL | BQL | BQL | BQL | BQL | BQL | BQL |

| | Time | | | | | | |
|---|---|---|---|---|---|---|---|
| | (h) | Lipid 1020 | | | Lipid 1021 | | |
| Liver | 6 | 36504 | 38824 | 43259 | 48135 | 33582 | 37570 |
| (ng/g) | 168 | 2894 | 2432 | 1960 | 3103 | 1228 | 1508 |

### CircRNA:

(331)
SEQ ID NO: 1
AACTTTTAGTTATCCCACAGCAAGAATGCCATCATCTGTCCTCACCCCCAATTTTCCCT
TTTCTTCCCCTGCAACCATTACGCTTACTCGCATGTGCATTGAGTGGTGCATGTGTTGA
ACAAACAGCTACACTCACATGGGGGCGGGTTTTCCCGCCCTACGGCCTCTCGCGAGGC
CCACCCCTTCCCTCCCCTTATAACTACAGTGCTTTGGTAGGTAAGCATCCTGATCCCCC
GCGGAAGCTGCTCACGTGGCAACTGTGGGGACCCAGACAGGTTATCAAAGGCACCCG
GTCTTTCCGCCTTCAGGAGTATCCCTACTAGTGAATTCTAGCGGGGCTCTGCTTGGTGC
CAACCTCCCCCAAATGCGCGCTGCGGGAGTGCTCTTCCCCAACTCACCCTAGTATCCTC
TCATGTGTGTGCTTGGTCAGCATATCTGAGACGATGTTCCGCTGTCCCAGACCAGTCCA
GTAATGGACGGGCCAGTGCGTGTAGTCGTCTTCCGGCTTGTCCGGGGCATGTTTGGTG
AACCGGTGGGGTAAGGTTGGTGTGCCCAACGCCCGTACTTTGGTGACACCTCAAGACC
ACCCAGGAATGCCAGGGAGGTACCCCACCTCACGGTGGGATCTGACCCTGGGCTAATT
GTCTACGGTGGTTCTTCTTGCTTCCACTTCTTTCTTCTGTTCACGGCCACCATGGAGGAC
GCCAAGAACATCAAGAAGGGCCCGGCGCCCTTCTACCCGCTGGAGGACGGGACCGCC
GGCGAGCAGCTCCACAAGGCCATGAAGCGGTACGCCCTGGTGCCGGGCACGATCGCC
TTCACCGACGCCCACATCGAGGTCGACATCACCTACGCGGAGTACTTCGAGATGAGCG
TGCGCCTGGCCGAGGCCATGAAGCGGTACGGCCTGAACACCAACCACCGGATCGTGGT
GTGCTCGGAGAACAGCCTGCAGTTCTTCATGCCGGTGCTGGGCGCCCTCTTCATCGGC
GTGGCCGTCGCCCCGGCGAACGACATCTACAACGAGCGGGAGCTGCTGAACAGCATG
GGGATCAGCCAGCCGACCGTGGTGTTCGTGAGCAAGAAGGGCCTGCAGAAGATCCTG
AACGTGCAGAAGAAGCTGCCCATCATCCAGAAGATCATCATCATGGACAGCAAGACC
GACTACCAGGGCTTCCAGTCGATGTACACGTTCGTGACCAGCCACCTCCCGCCGGGCT
TCAACGAGTACGACTTCGTCCCGGAGAGCTTCGACCGGGACAAGACCATCGCCCTGAT
CATGAACAGCAGCGGCAGCACCGGCCTGCCGAAGGGGGTGGCCCTGCCGCACCGGAC
CGCCTGCGTGCGCTTCTCGCACGCCCGGGACCCCATCTTCGGCAACCAGATCATCCCG
GACACCGCCATCCTGAGCGTGGTGCCGTTCCACCACGGCTTCGGCATGTTCACGACCC
TGGGCTACCTCATCTGCGGCTTCCGGGTGGTCCTGATGTACCGGTTCGAGGAGGAGCT
GTTCCTGCGGAGCCTGCAGGACTACAAGATCCAGAGCGCGCTGCTCGTGCCGACCCTG
TTCAGCTTCTTCGCCAAGAGCACCCTGATCGACAAGTACGACCTGTCGAACCTGCACG
AGATCGCCAGCGGGGGCGCCCCGCTGAGCAAGGAGGTGGGCGAGGCCGTGGCCAAGC
GGTTCCACCTCCCGGGCATCCGCCAGGGCTACGGCCTGACCGAGACCACGAGCGCGAT
CCTGATCACCCCCGAGGGGGACGACAAGCCGGGCGCCGTGGGCAAGGTGGTCCCGTT
CTTCGAGGCCAAGGTGGTGGACCTGGACACCGGCAAGACCCTGGGCGTGAACCAGCG
GGGCGAGCTGTGCGTGCGGGGGCCGATGATCATGAGCGGCTACGTGAACAACCCGGA
GGCCACCAACGCCCTCATCGACAAGGACGGCTGGCTGCACAGCGGCGACATCGCCTAC
TGGGACGAGGACGAGCACTTCTTCATCGTCGACCGGCTGAAGTCGCTGATCAAGTACA
AGGGCTACCAGGTGGCGCCGGCCGAGCTGGAGAGCATCCTGCTCCAGCACCCCAACAT
CTTCGACGCCGGCGTGGCCGGGCTGCCGGACGACGACGCCGGCGAGCTGCCGGCCGC
GGTGGTGGTGCTGGAGCACGGCAAGACCATGACGGAGAAGGAGATCGTCGACTACGT
GGCCAGCCAGGTGACCACCGCCAAGAAGCTGCGGGGCGGCGTGGTGTTCGTGGACGA
GGTCCCGAAGGGCCTGACCGGGAAGCTCGACGCCCGGAAGATCCGCGAGATCCTGAT
CAAGGCCAAGAAGGGCGGCAAGATCGCCGTGAGCTGATAAACTACTGAAAGCAT
(019)
SEQ ID NO: 2
AAAATCCGTTGACCCACAGCAAGAATGCCATCATCTGTCCTCACCCCCAATTTTCCCTT
TTCTTCCCCTGCAACCATTACGCTTACTCGCATGTGCATTGAGTGGTGCATGTGTTGAA
CAAACAGCTACACTCACATGGGGGCGGGTTTTCCCGCCCTACGGCCTCTCGCGAGGCC
CACCCCTTCCCTCCCCTTATAACTACAGTGCTTTGGTAGGTAAGCATCCTGATCCCCCG
CGGAAGCTGCTCACGTGGCAACTGTGGGGACCCAGACAGGTTATCAAAGGCACCCGG
TCTTTCCGCCTTCAGGAGTATCCCTACTAGTGAATTCTAGCGGGGCTCTGCTTGGTGCC
AACCTCCCCCAAATGCGCGCTGCGGGAGTGCTCTTCCCCAACTCACCCTAGTATCCTCT
CATGTGTGTGCTTGGTCAGCATATCTGAGACGATGTTCCGCTGTCCCAGACCAGTCCAG
TAATGGACGGGCCAGTGCGTGTAGTCGTCTTCCGGCTTGTCCGGGGCATGTTTGGTGA
ACCGGTGGGGTAAGGTTGGTGTGCCCAACGCCCGTACTTTGGTGACACCTCAAGACCA
CCCAGGAATGCCAGGGAGGTACCCCACCTCACGGTGGGATCTGACCCTGGGCTAATTG
TCTACGGTGGTTCTTCTTGCTTCCACTTCTTTCTTCTGTTCACGGCCACCATGGGAGTGC
ATGAATGTCCTGCCTGGCTGTGGCTTCTCCTGTCACTGCTGTCTCTCCCTCTGGGCCTCC
CAGTGCTGGGCGCACCACCAAGACTCATCTGTGACAGCAGAGTGCTGGAGAGGTATCT
CTTGGAGGCCAAGGAGGCTGAGAACATTACCACAGGCTGTGCTGAACACTGCAGCTTG
AATGAGAATATCACTGTCCCAGACACCAAAGTTAATTTCTATGCCTGGAAGAGGATGG
AGGTTGGGCAACAAGCAGTTGAAGTGTGGCAAGGCCTGGCCCTGCTGTCTGAAGCTGT
CCTGAGGGGCCAGGCACTGTTGGTCAACTCTTCCCAGCCTTGGGAGCCCCTGCAACTG
CATGTGGATAAAGCAGTGAGTGGCCTTAGAAGCCTCACCACTCTGCTTCGGGCTCTGG
GAGCACAGAAGGAAGCCATCTCCCCTCCAGATGCAGCCTCAGCAGCTCCACTCAGAAC
AATTACTGCTGACACTTTTAGAAAACTCTTTAGGGTGTACTCCAATTTCCTCCGGGGAA
AGCTGAAGCTGTACACAGGTGAGGCATGTAGGACAGGGGACAGATAATAGTGAAGAC
GCTACGGACTT

1-39. (canceled)

40. A cationic lipid represented by the structure of formula (II): 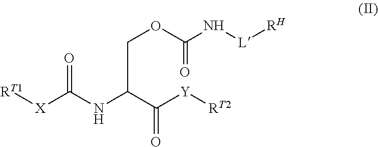
or a salt, hydrate, solvate, polymorph, optical isomer, geometrical isomer, enantiomer, diastereomer, tautomer, isotope labeled compound or mixtures thereof,
wherein:
L′ is —C₁₋₆ alkylene-;
X is O;
Y is O;
R^{H} is NR⁴R⁵, wherein R⁴ and R⁵ together with the nitrogen to which they are attached form a 4- to 10-membered heterocyclic ring, optionally containing one or more additional heteroatoms selected from the group consisting of O, N and S;
R^{T1} and R^{T2} are each independently selected from the group consisting of:
(a) C₁₀-C₂₂ alkyl;
(b) C₁₀-C₂₂ alkenyl;
(c) C₁₀-C₂₂ alkynyl;
(d) C₄-C₁₅ alkylene-Z—C₄-C₂₂ alkyl; and
(e) C₄-C₁₅ alkylene-Z—C₄-C₂₂ alkenyl;
Z is —O—C(═O)—, —C(═O)—O— or —O—.

41. The cationic lipid according to claim 40, or a salt, hydrate, solvate, polymorph, optical isomer, geometrical isomer, enantiomer, diastereomer, tautomer, isotope labeled compound or mixtures thereof, wherein L′ is —(CH₂)ₙ—, and wherein n=1, 2 or 3.

42. The cationic lipid according to claim 41, or a salt, hydrate, solvate, polymorph, optical isomer, geometrical isomer, enantiomer, diastereomer, tautomer, isotope labeled compound or mixtures thereof, wherein n=2.

43. The cationic lipid according to claim 40, or a salt, hydrate, solvate, polymorph, optical isomer, geometrical isomer, enantiomer, diastereomer, tautomer, isotope labeled compound or mixtures thereof, wherein R^{H} is

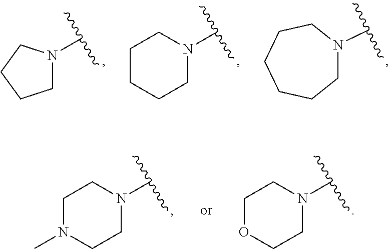

44. The cationic lipid according to claim 43, or a salt, hydrate, solvate, polymorph, optical isomer, geometrical isomer, enantiomer, diastereomer, tautomer, isotope labeled compound or mixtures thereof, wherein R^{H} is

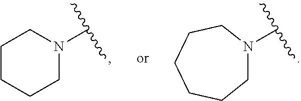 [SMILES: *N1CCCCC1 ; *N1CCCCCC1]

45. The cationic lipid according to claim 40, or a salt, hydrate, solvate, polymorph, optical isomer, geometrical isomer, enantiomer, diastereomer, tautomer, isotope labeled compound or mixtures thereof, wherein R^{T1} and R^{T2} are each independently selected from the group consisting of:

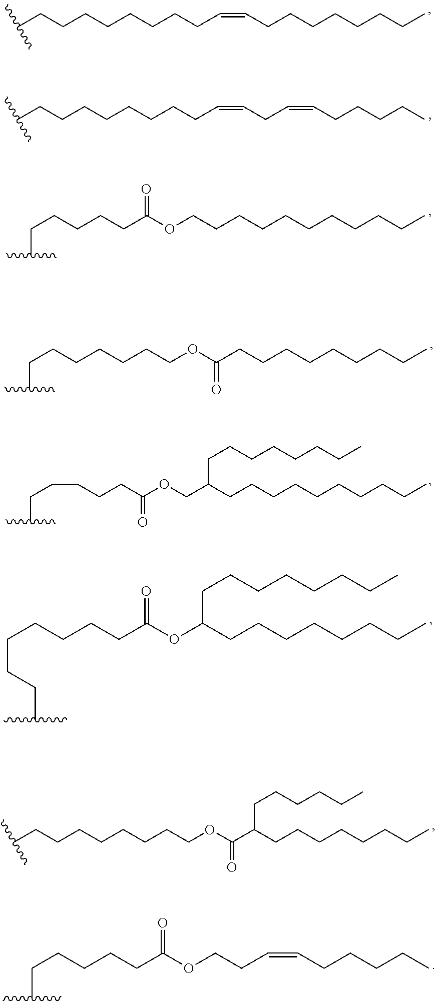

46. The cationic lipid according to claim 45, or a salt, hydrate, solvate, polymorph, optical isomer, geometrical isomer, enantiomer, diastereomer, tautomer, isotope labeled compound or mixtures thereof, wherein R^{T1} and R^{T2} are each

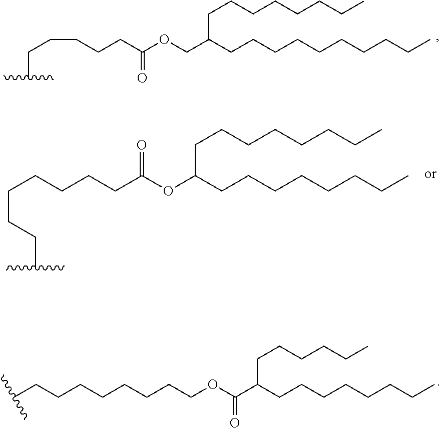 [SMILES: *CCCCCC(=O)OCC(CCCCCCCC)CCCCCCCCCC ; *CCCCCCCC(=O)OC(CCCCCCCC)CCCCCCCC ; *CCCCCCCCOC(=O)C(CCCCCC)CCCCCCCC]

47. The cationic lipid according to claim 40, or a salt, hydrate, solvate, polymorph, optical isomer, geometrical isomer, enantiomer, diastereomer, tautomer, isotope labeled compound or mixtures thereof, wherein said cationic lipid has the following structures:

| No. | Structure |
|---|---|
| 888. | 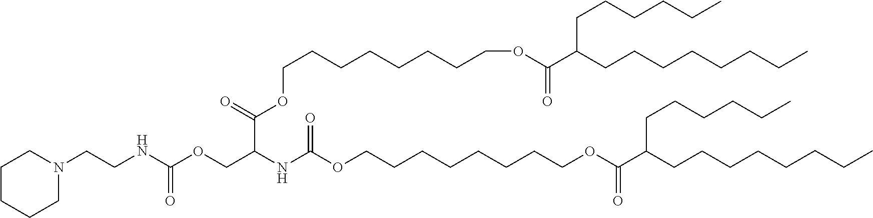 [SMILES: CCCCCCCCC(CCCCCC)C(=O)OCCCCCCCCOC(=O)NC(COC(=O)NCCN1CCCCC1)C(=O)OCCCCCCCCOC(=O)C(CCCCCC)CCCCCCCC] |
| 889. | 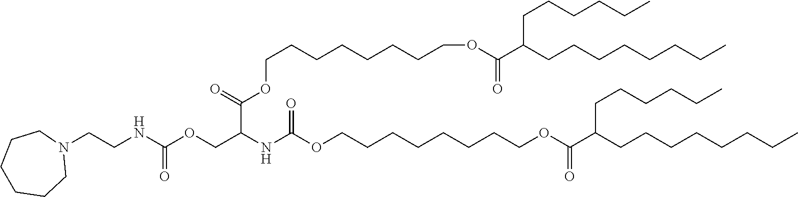 |
| 896. | 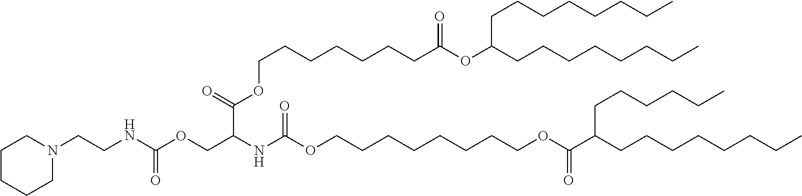 [SMILES: CCCCCCCCC(CCCCCCCC)OC(=O)CCCCCCCOC(=O)C(COC(=O)NCCN1CCCCC1)NC(=O)OCCCCCCCCOC(=O)C(CCCCCC)CCCCCCCC] |
| 897. | 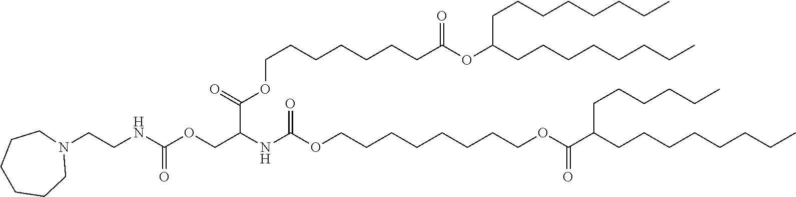 [SMILES: CCCCCCCCC(CCCCCCCC)OC(=O)CCCCCCCOC(=O)C(COC(=O)NCCN1CCCCCC1)NC(=O)OCCCCCCCCOC(=O)C(CCCCCC)CCCCCCCC] |
| 904. | 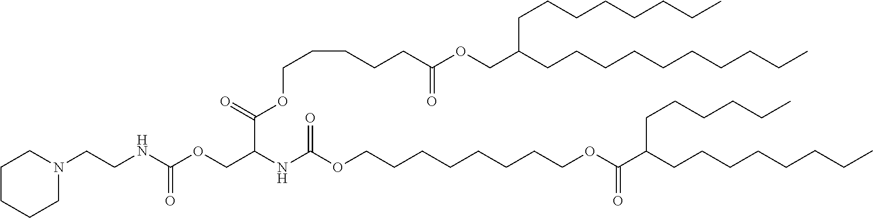 [SMILES: CCCCCCCCCCC(CCCCCCCC)COC(=O)CCCCCOC(=O)C(COC(=O)NCCN1CCCCC1)NC(=O)OCCCCCCCCOC(=O)C(CCCCCC)CCCCCCCC] |
| 905. | 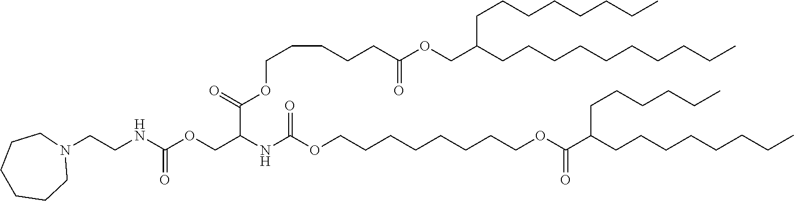 [SMILES: CCCCCCCCCCC(CCCCCCCC)COC(=O)CCCCCOC(=O)C(COC(=O)NCCN1CCCCCC1)NC(=O)OCCCCCCCCOC(=O)C(CCCCCC)CCCCCCCC] |
| 1015. | 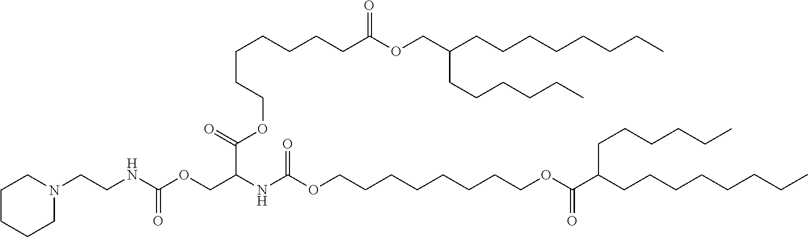 [SMILES: CCCCCCCCC(CCCCCC)COC(=O)CCCCCCCOC(=O)C(COC(=O)NCCN1CCCCC1)NC(=O)OCCCCCCCCOC(=O)C(CCCCCC)CCCCCCCC] |
| 1016. | 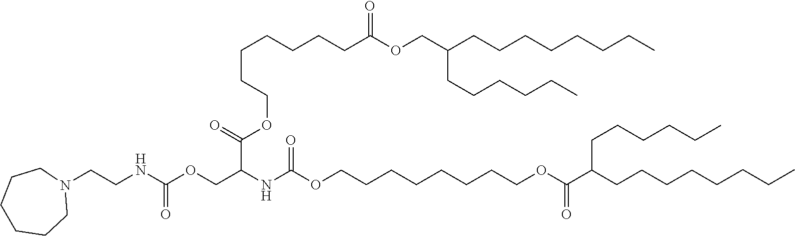 [SMILES: CCCCCCCCC(CCCCCC)COC(=O)CCCCCCCOC(=O)C(COC(=O)NCCN1CCCCCC1)NC(=O)OCCCCCCCCOC(=O)C(CCCCCC)CCCCCCCC] |
| 1020. | 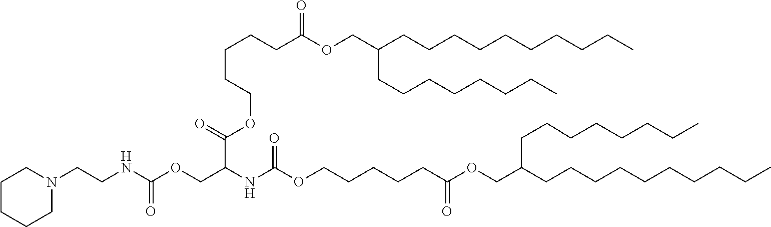 [SMILES: CCCCCCCCCCC(CCCCCCCC)COC(=O)CCCCCOC(=O)NC(COC(=O)NCCN1CCCCC1)C(=O)OCCCCCC(=O)OCC(CCCCCCCC)CCCCCCCCCC] |
| 1021. | 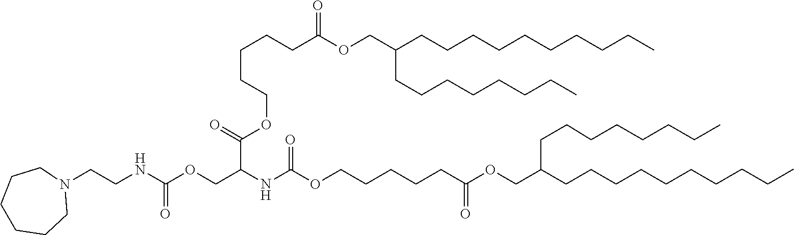 [SMILES: CCCCCCCCCCC(CCCCCCCC)COC(=O)CCCCCOC(=O)NC(COC(=O)NCCN1CCCCCC1)C(=O)OCCCCCC(=O)OCC(CCCCCCCC)CCCCCCCCCC] |
| 1030. | 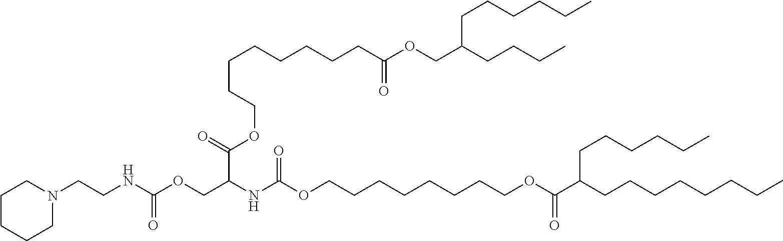 [SMILES: CCCCCCCCC(CCCCCC)C(=O)OCCCCCCCCOC(=O)NC(COC(=O)NCCN1CCCCC1)C(=O)OCCCCCCCCC(=O)OCC(CCCC)CCCCCC] |
| 1031. | 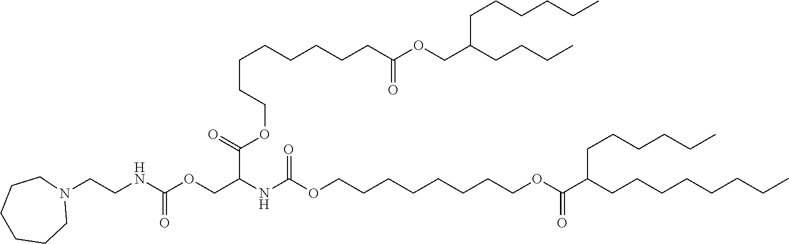 [SMILES: CCCCCCCCC(CCCCCC)C(=O)OCCCCCCCCOC(=O)NC(COC(=O)NCCN1CCCCCC1)C(=O)OCCCCCCCCC(=O)OCC(CCCC)CCCCCC] |
| 1071. | 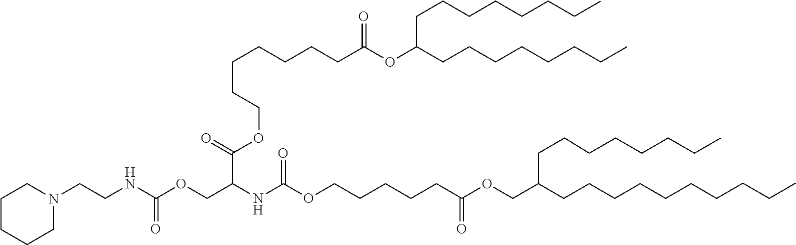 [SMILES: CCCCCCCCCCC(CCCCCCCC)COC(=O)CCCCCOC(=O)NC(COC(=O)NCCN1CCCCC1)C(=O)OCCCCCCCC(=O)OC(CCCCCCCC)CCCCCCCC] |
| 1072. | 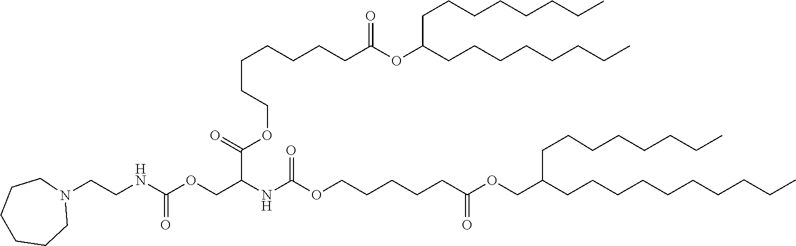 [SMILES: CCCCCCCCCCC(CCCCCCCC)COC(=O)CCCCCOC(=O)NC(COC(=O)NCCN1CCCCCC1)C(=O)OCCCCCCCC(=O)OC(CCCCCCCC)CCCCCCCC] |
| 1086. | 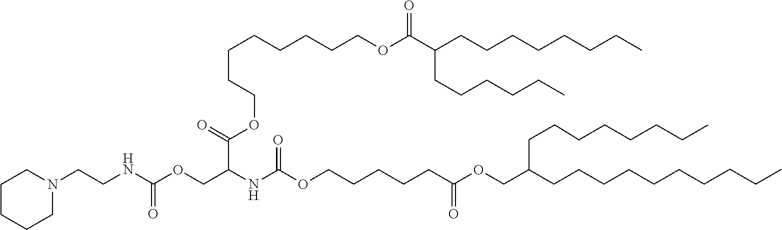 [SMILES: CCCCCCCCCCC(CCCCCCCC)COC(=O)CCCCCOC(=O)NC(COC(=O)NCCN1CCCCC1)C(=O)OCCCCCCCCOC(=O)C(CCCCCC)CCCCCCCC] |
| 1087. | 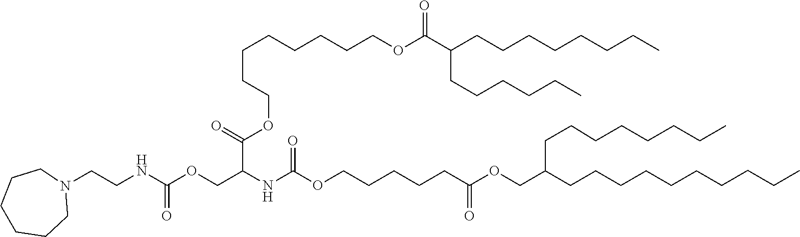 [SMILES: CCCCCCCCCCC(CCCCCCCC)COC(=O)CCCCCOC(=O)NC(COC(=O)NCCN1CCCCCC1)C(=O)OCCCCCCCCOC(=O)C(CCCCCC)CCCCCCCC] |
| 1090. | 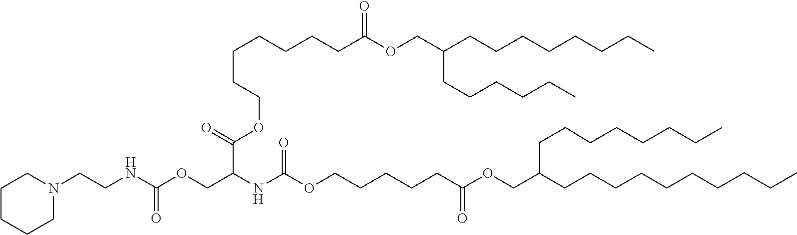 [SMILES: CCCCCCCCCCC(CCCCCCCC)COC(=O)CCCCCOC(=O)NC(COC(=O)NCCN1CCCCC1)C(=O)OCCCCCCCC(=O)OCC(CCCCCC)CCCCCCCC] |
| 1091. | 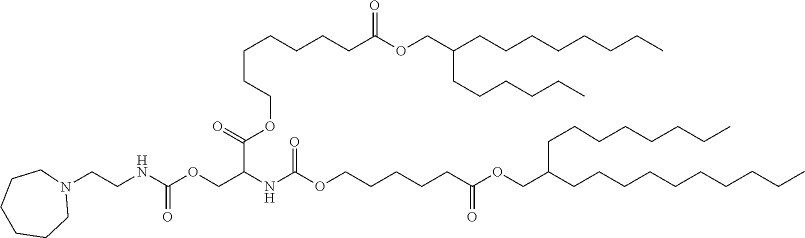 [SMILES: CCCCCCCCCCC(CCCCCCCC)COC(=O)CCCCCOC(=O)NC(COC(=O)NCCN1CCCCCC1)C(=O)OCCCCCCCC(=O)OCC(CCCCCC)CCCCCCCC] |
| 1100. | 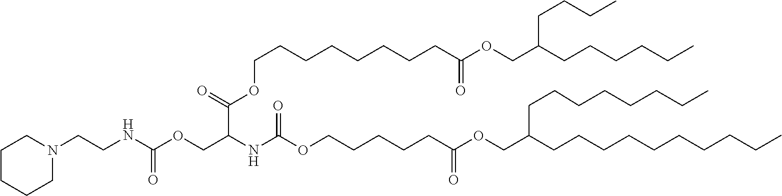 [SMILES: CCCCCCCCCCC(CCCCCCCC)COC(=O)CCCCCOC(=O)NC(COC(=O)NCCN1CCCCC1)C(=O)OCCCCCCCCC(=O)OCC(CCCC)CCCCCC] |
| 1101. | 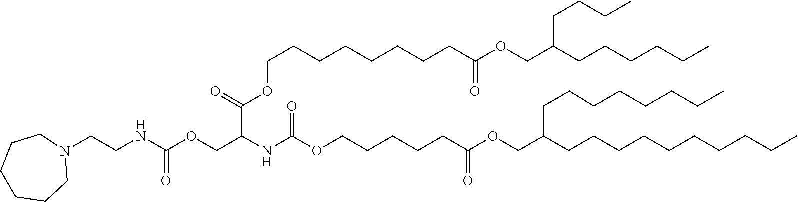 [SMILES: CCCCCCCCCCC(CCCCCCCC)COC(=O)CCCCCOC(=O)NC(COC(=O)NCCN1CCCCCC1)C(=O)OCCCCCCCCC(=O)OCC(CCCC)CCCCCC] |
| 1129. | 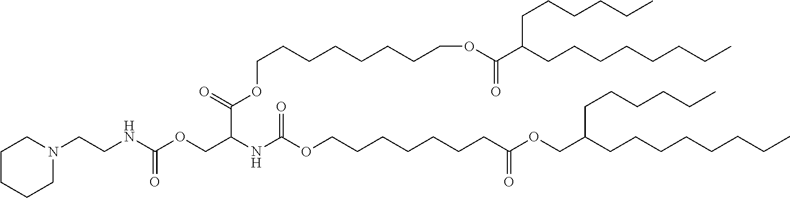 [SMILES: CCCCCCCCC(CCCCCC)COC(=O)CCCCCCCOC(=O)NC(COC(=O)NCCN1CCCCC1)C(=O)OCCCCCCCCOC(=O)C(CCCCCC)CCCCCCCC] |
| 1130. | 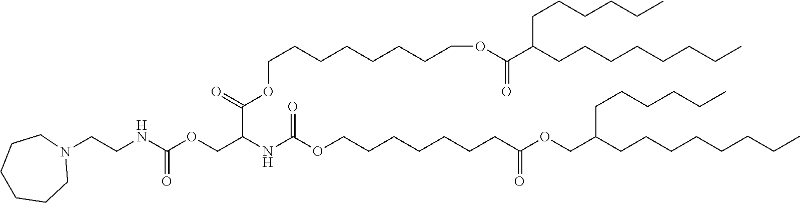 [SMILES: CCCCCCCCC(CCCCCC)COC(=O)CCCCCCCOC(=O)NC(COC(=O)NCCN1CCCCCC1)C(=O)OCCCCCCCCOC(=O)C(CCCCCC)CCCCCCCC] |
| 1133. | 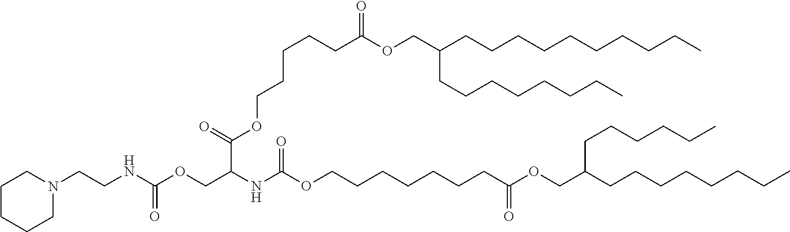 [SMILES: CCCCCCCCCCC(CCCCCCCC)COC(=O)CCCCCOC(=O)C(COC(=O)NCCN1CCCCC1)NC(=O)OCCCCCCCC(=O)OCC(CCCCCC)CCCCCCCC] |
| 1134. | 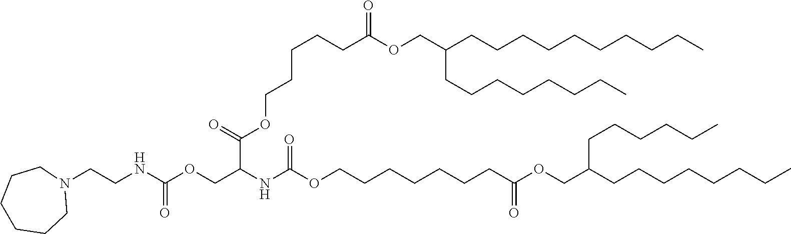 [SMILES: CCCCCCCCCCC(CCCCCCCC)COC(=O)CCCCCOC(=O)C(COC(=O)NCCN1CCCCCC1)NC(=O)OCCCCCCCC(=O)OCC(CCCCCC)CCCCCCCC] |
| 1137. | 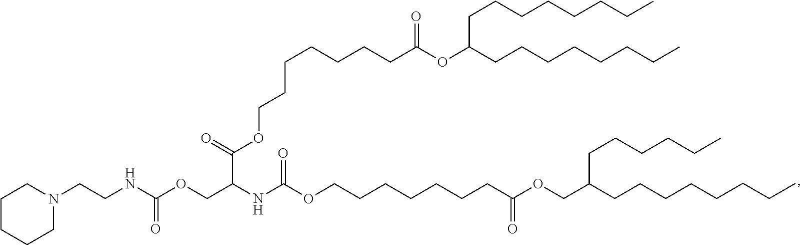 [SMILES: CCCCCCCCC(CCCCCC)COC(=O)CCCCCCCOC(=O)NC(COC(=O)NCCN1CCCCC1)C(=O)OCCCCCCCC(=O)OC(CCCCCCCC)CCCCCCCC] |
| | or |
| 1138. | 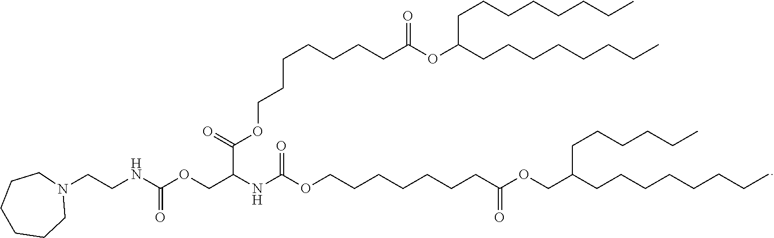 [SMILES: CCCCCCCCC(CCCCCC)COC(=O)CCCCCCCOC(=O)NC(COC(=O)NCCN1CCCCCC1)C(=O)OCCCCCCCC(=O)OC(CCCCCCCC)CCCCCCCC] |

48. A pharmaceutical composition comprising the cationic lipid according to claim 40, or a salt, hydrate, solvate, polymorph, optical isomer, geometrical isomer, enantiomer, diastereomer, tautomer, isotope labeled compound or mixtures thereof, and a pharmaceutically acceptable carrier.

49. The pharmaceutical composition according to claim 48, wherein said cationic lipid has the following structure:

| No. | Structure |
|---|---|
| 888. | 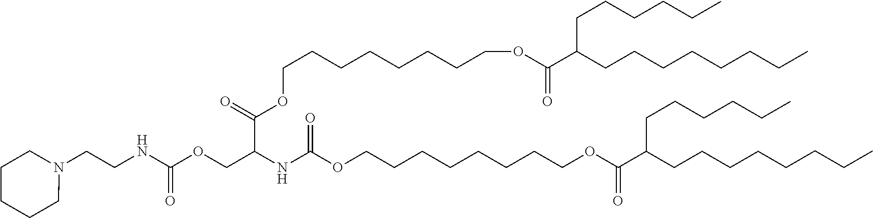 [SMILES: CCCCCCCCC(CCCCCC)C(=O)OCCCCCCCCOC(=O)NC(COC(=O)NCCN1CCCCC1)C(=O)OCCCCCCCCOC(=O)C(CCCCCC)CCCCCCCC] |
| 889. | 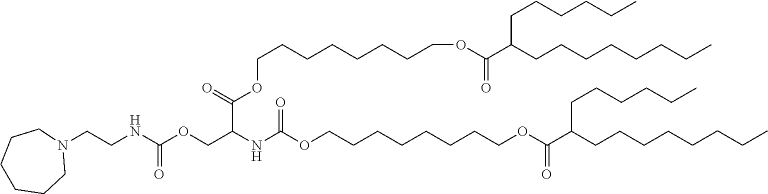 [SMILES: CCCCCCCCC(CCCCCC)C(=O)OCCCCCCCCOC(=O)NC(COC(=O)NCCN1CCCCCC1)C(=O)OCCCCCCCCOC(=O)C(CCCCCC)CCCCCCCC] |
| 896. | 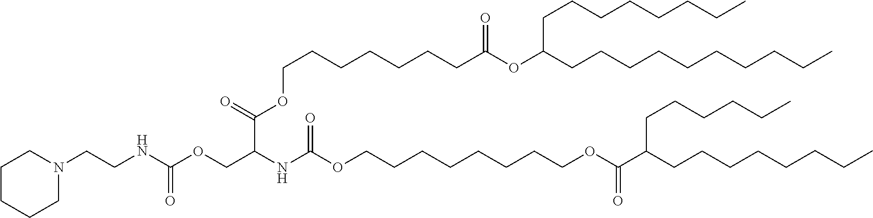 [SMILES: CCCCCCCCCCC(CCCCCCCC)OC(=O)CCCCCCCOC(=O)C(COC(=O)NCCN1CCCCC1)NC(=O)OCCCCCCCCOC(=O)C(CCCCCC)CCCCCCCC] |
| 897. | 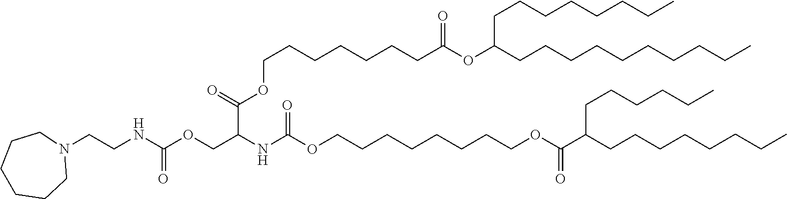 [SMILES: CCCCCCCCCCC(CCCCCCCC)OC(=O)CCCCCCCOC(=O)C(COC(=O)NCCN1CCCCCC1)NC(=O)OCCCCCCCCOC(=O)C(CCCCCC)CCCCCCCC] |
| 904. | 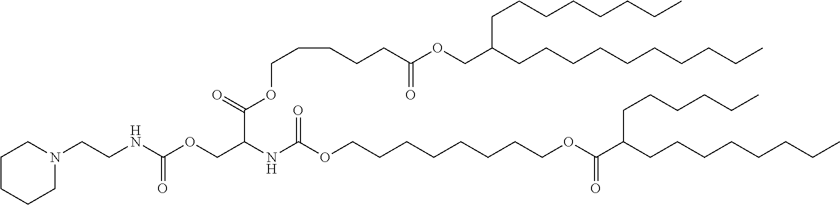 |
| 905. | 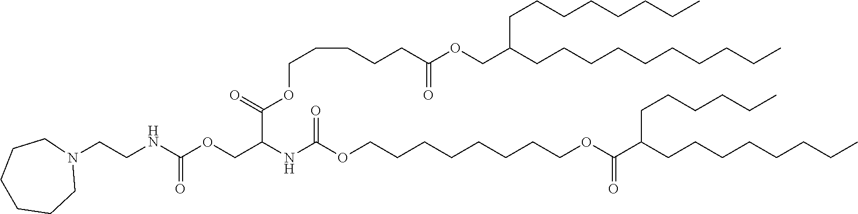 |
| 1015. | 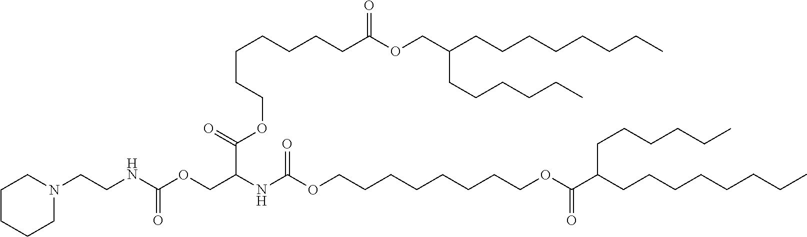 [SMILES: CCCCCCCCC(CCCCCC)COC(=O)CCCCCCCOC(=O)C(COC(=O)NCCN1CCCCC1)NC(=O)OCCCCCCCCOC(=O)C(CCCCCC)CCCCCCCC] |
| 1016. | 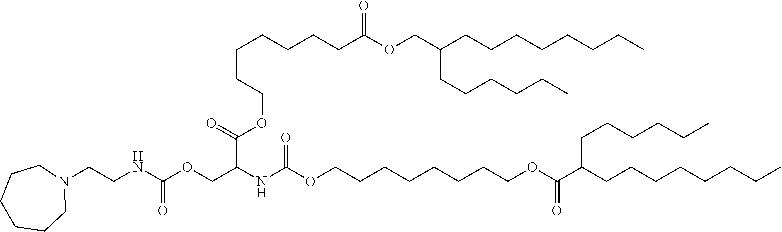 [SMILES: CCCCCCCCC(CCCCCC)COC(=O)CCCCCCCOC(=O)C(COC(=O)NCCN1CCCCCC1)NC(=O)OCCCCCCCCOC(=O)C(CCCCCC)CCCCCCCC] |
| 1020. | 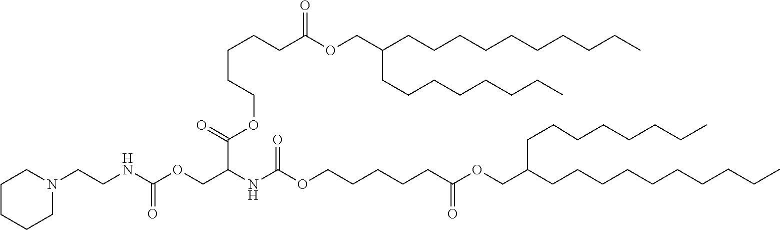 [SMILES: CCCCCCCCCCC(CCCCCCCC)COC(=O)CCCCCOC(=O)NC(COC(=O)NCCN1CCCCC1)C(=O)OCCCCCC(=O)OCC(CCCCCCCC)CCCCCCCCCC] |
| 1021. | 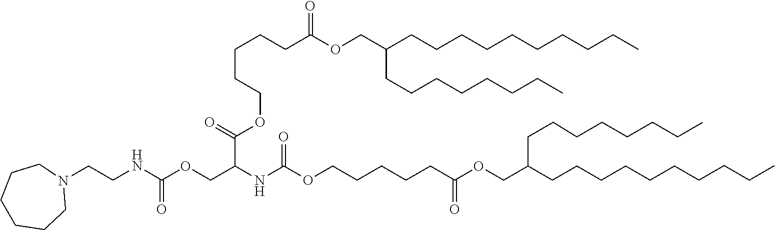 [SMILES: CCCCCCCCCCC(CCCCCCCC)COC(=O)CCCCCOC(=O)NC(COC(=O)NCCN1CCCCCC1)C(=O)OCCCCCC(=O)OCC(CCCCCCCC)CCCCCCCCCC] |
| 1030. | 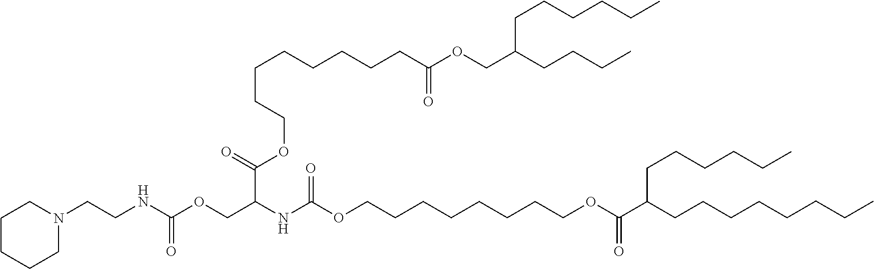 [SMILES: CCCCCCCCC(CCCCCC)C(=O)OCCCCCCCCOC(=O)NC(COC(=O)NCCN1CCCCC1)C(=O)OCCCCCCCCC(=O)OCC(CCCC)CCCCCC] |
| 1031. | 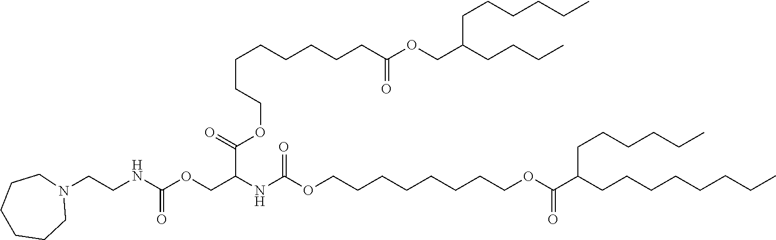 [SMILES: CCCCCCCCC(CCCCCC)C(=O)OCCCCCCCCOC(=O)NC(COC(=O)NCCN1CCCCCC1)C(=O)OCCCCCCCCC(=O)OCC(CCCC)CCCCCC] |
| 1071. | 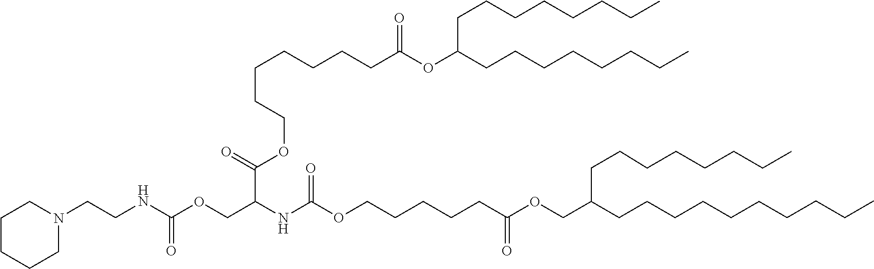 [SMILES: CCCCCCCCCCC(CCCCCCCC)COC(=O)CCCCCOC(=O)NC(COC(=O)NCCN1CCCCC1)C(=O)OCCCCCCCC(=O)OC(CCCCCCCC)CCCCCCCC] |
| 1072. | 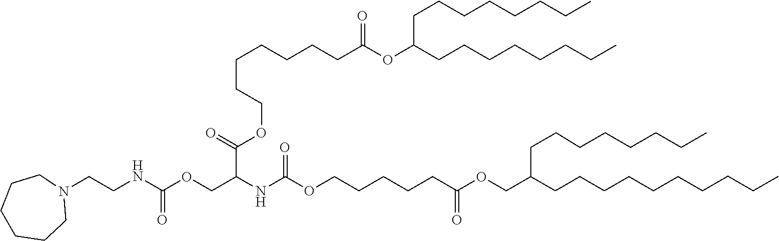 [SMILES: CCCCCCCCCCC(CCCCCCCC)COC(=O)CCCCCOC(=O)NC(COC(=O)NCCN1CCCCCC1)C(=O)OCCCCCCCC(=O)OC(CCCCCCCC)CCCCCCCC] |
| 1086. | 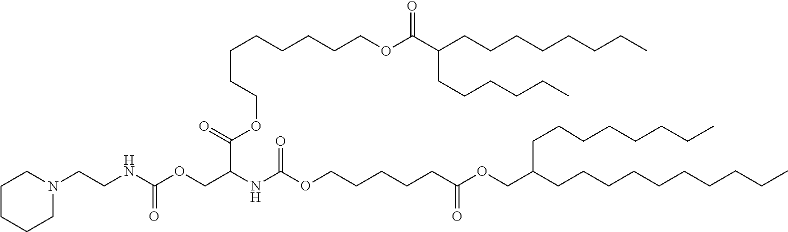 [SMILES: CCCCCCCCCCC(CCCCCCCC)COC(=O)CCCCCOC(=O)NC(COC(=O)NCCN1CCCCC1)C(=O)OCCCCCCCCOC(=O)C(CCCCCC)CCCCCCCC] |
| 1087. | 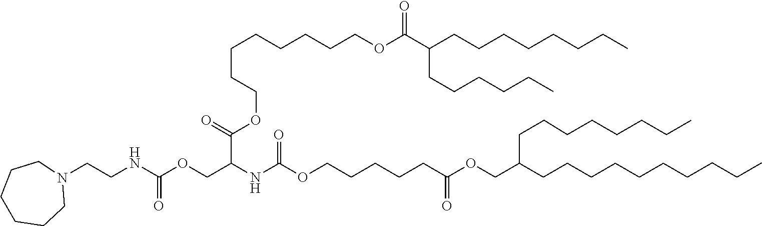 [SMILES: CCCCCCCCCCC(CCCCCCCC)COC(=O)CCCCCOC(=O)NC(COC(=O)NCCN1CCCCCC1)C(=O)OCCCCCCCCOC(=O)C(CCCCCC)CCCCCCCC] |
| 1090. | 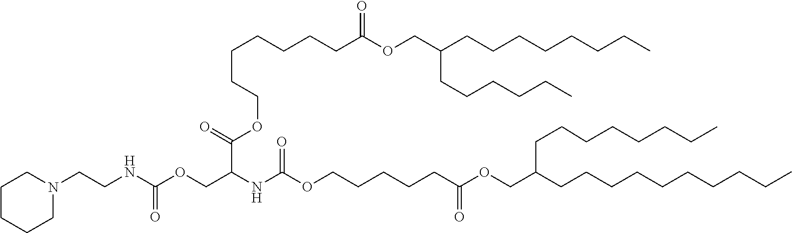 [SMILES: CCCCCCCCCCC(CCCCCCCC)COC(=O)CCCCCOC(=O)NC(COC(=O)NCCN1CCCCC1)C(=O)OCCCCCCCC(=O)OCC(CCCCCC)CCCCCCCC] |
| 1091. | 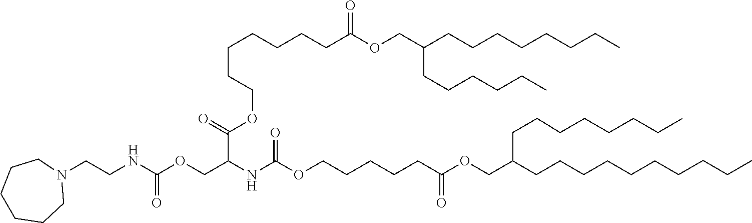 [SMILES: CCCCCCCCCCC(CCCCCCCC)COC(=O)CCCCCOC(=O)NC(COC(=O)NCCN1CCCCCC1)C(=O)OCCCCCCCC(=O)OCC(CCCCCC)CCCCCCCC] |
| 1100. | 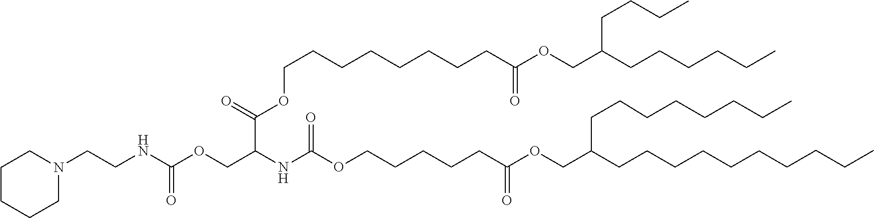 [SMILES: CCCCCCCCCCC(CCCCCCCC)COC(=O)CCCCCOC(=O)NC(COC(=O)NCCN1CCCCC1)C(=O)OCCCCCCCCC(=O)OCC(CCCC)CCCCCC] |
| 1101. | 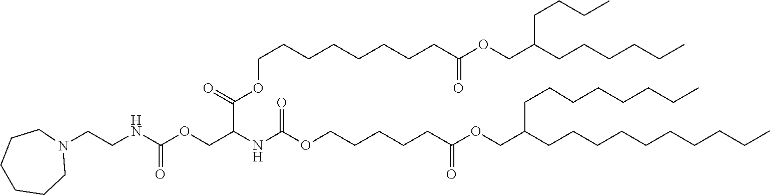 [SMILES: CCCCCCCCCCC(CCCCCCCC)COC(=O)CCCCCOC(=O)NC(COC(=O)NCCN1CCCCCC1)C(=O)OCCCCCCCCC(=O)OCC(CCCC)CCCCCC] |
| 1129. | 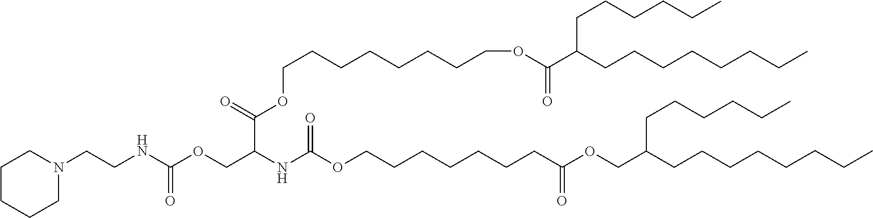 [SMILES: CCCCCCCCC(CCCCCC)COC(=O)CCCCCCCOC(=O)NC(COC(=O)NCCN1CCCCC1)C(=O)OCCCCCCCCOC(=O)C(CCCCCC)CCCCCCCC] |
| 1130. | 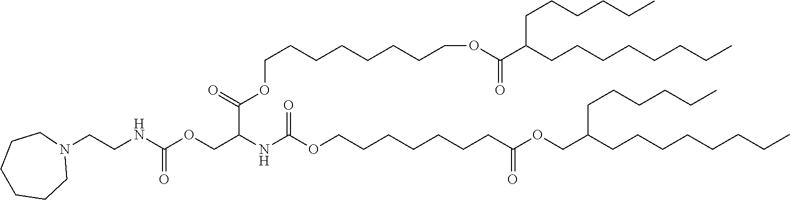 [SMILES: CCCCCCCCC(CCCCCC)COC(=O)CCCCCCCOC(=O)NC(COC(=O)NCCN1CCCCCC1)C(=O)OCCCCCCCCOC(=O)C(CCCCCC)CCCCCCCC] |
| 1133. | 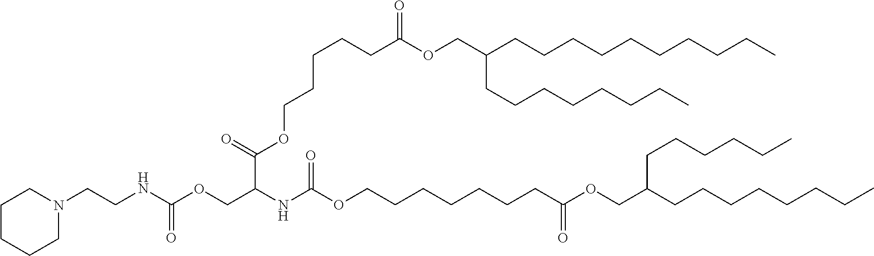 [SMILES: CCCCCCCCCCC(CCCCCCCC)COC(=O)CCCCCOC(=O)C(COC(=O)NCCN1CCCCC1)NC(=O)OCCCCCCCC(=O)OCC(CCCCCC)CCCCCCCC] |
| 1134. | 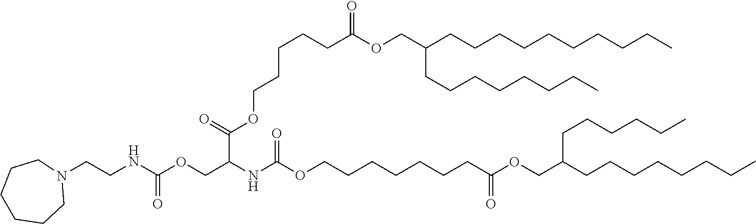 [SMILES: CCCCCCCCCCC(CCCCCCCC)COC(=O)CCCCCOC(=O)C(COC(=O)NCCN1CCCCCC1)NC(=O)OCCCCCCCC(=O)OCC(CCCCCC)CCCCCCCC] |
| 1137. | 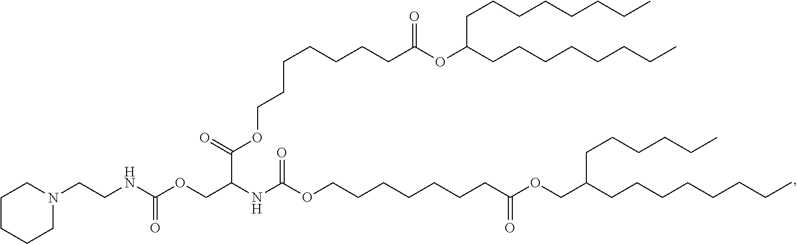 [SMILES: CCCCCCCCC(CCCCCC)COC(=O)CCCCCCCOC(=O)NC(COC(=O)NCCN1CCCCC1)C(=O)OCCCCCCCC(=O)OC(CCCCCCCC)CCCCCCCC] |
| | or |
| 1138. | 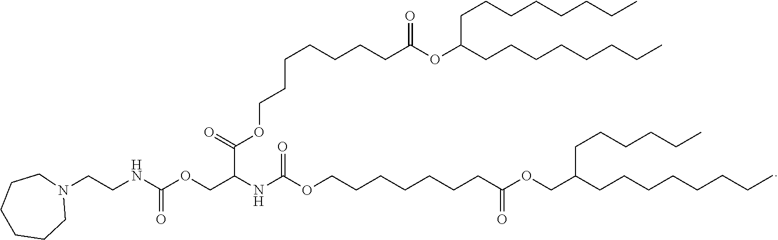 [SMILES: CCCCCCCCC(CCCCCC)COC(=O)CCCCCCCOC(=O)NC(COC(=O)NCCN1CCCCCC1)C(=O)OCCCCCCCC(=O)OC(CCCCCCCC)CCCCCCCC] |


SEQUENCE LISTING (2 sequences)

SEQ ID NO: 1 - RNA, 2367 nt, synthetic construct
  source 1..2367: mol_type = other RNA
aacttttagttatcccacagcaagaatgccatcatctgtcctcacccccaattttcccttttcttcccctgcaaccattacgcttactcgcatgtgcattgagtggtgcatgtgttgaacaaacagctacactcacatgggggcgggttttcccgccctacggcctctcgcgaggcccaccccttccctccccttataactacagtgctttggtaggtaagcatcctgatcccccgcggaagctgctcacgtggcaactgtggggacccagacaggttatcaaaggcacccggtctttccgccttcaggagtatccctactagtgaattctagcggggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgcgtgtagtcgtcttccggcttgtccggggcatgtttggtgaaccggtggggtaaggttggtgtgcccaacgcccgtactttggtgacacctcaagaccacccaggaatgccagggaggtaccccacctcacggtgggatctgaccctgggctaattgtctacggtggttcttcttgcttccacttctttcttctgttcacggccaccatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgagctgataaactactgaaagcat

SEQ ID NO: 2 - RNA, 1296 nt, synthetic construct
  source 1..1296: mol_type = other RNA
aaaatccgttgacccacagcaagaatgccatcatctgtcctcacccccaattttcccttttcttcccctgcaaccattacgcttactcgcatgtgcattgagtggtgcatgtgttgaacaaacagctacactcacatgggggcgggttttcccgccctacggcctctcgcgaggcccaccccttccctccccttataactacagtgctttggtaggtaagcatcctgatcccccgcggaagctgctcacgtggcaactgtggggacccagacaggttatcaaaggcacccggtctttccgccttcaggagtatccctactagtgaattctagcggggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgcgtgtagtcgtcttccggcttgtccggggcatgtttggtgaaccggtggggtaaggttggtgtgcccaacgcccgtactttggtgacacctcaagaccacccaggaatgccagggaggtaccccacctcacggtgggatctgaccctgggctaattgtctacggtggttcttcttgcttccacttctttcttctgttcacggccaccatgggagtgcatgaatgtcctgcctggctgtggcttctcctgtcactgctgtctctccctctgggcctcccagtgctgggcgcaccaccaagactcatctgtgacagcagagtgctggagaggtatctcttggaggccaaggaggctgagaacattaccacaggctgtgctgaacactgcagcttgaatgagaatatcactgtcccagacaccaaagttaatttctatgcctggaagaggatggaggttgggcaacaagcagttgaagtgtggcaaggcctggccctgctgtctgaagctgtcctgaggggccaggcactgttggtcaactcttcccagccttgggagcccctgcaactgcatgtggataaagcagtgagtggccttagaagcctcaccactctgcttcgggctctgggagcacagaaggaagccatctcccctccagatgcagcctcagcagctccactcagaacaattactgctgacacttttagaaaactctttagggtgtactccaatttcctccggggaaagctgaagctgtacacaggtgaggcatgtaggacaggggacagataatagtgaagacgctacggactt